(19) United States
(12) Patent Application Publication
(10) Pub. No.: US 2025/0351667 A1
(43) Pub. Date: Nov. 13, 2025

(54) NOVEL COMPOUND AND ORGANIC LIGHT EMITTING DEVICE COMPRISING THE SAME

(71) Applicant: LG CHEM, LTD., Seoul (KR)

(72) Inventors: Minjun Kim, Daejeon (KR); Sung Kil Hong, Daejeon (KR); Sung Jae Lee, Daejeon (KR); Hyeon Jin Mun, Daejeon (KR); Yonghan Lee, Daejeon (KR)

(21) Appl. No.: 18/869,433

(22) PCT Filed: Oct. 11, 2023
(86) PCT No.: PCT/KR2023/015639

§ 371 Date: Nov. 26, 2024

(30) Foreign Application Priority Data
Oct. 11, 2022 (KR) 10-2022-0129527
Oct. 11, 2023 (KR) 10-2023-0135250

(51) Int. Cl. H10K 50/15; C07D 307/91; H10K 85/60

(52) U.S. Cl. CPC H10K 50/15; C07D 307/91; H10K 85/622; H10K 85/626; H10K 85/633; H10K 85/636; H10K 85/654; H10K 85/6572; H10K 85/6574

(57) ABSTRACT

A novel compound of Chemical Formula 1:

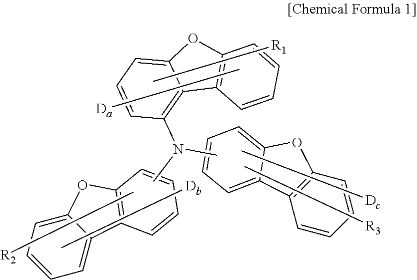

where D is deuterium; a to c are each independently an integer of 0 to 6; R₁ to R₃ are each independently hydrogen, deuterium, or -Lₐ-Rₐ, with the proviso that at least one of R₁ to R₃ is -Lₐ-Rₐ; Lₐ is a single bond; or a substituted or unsubstituted C₆₋₃₀ arylene; Rₐ is a substituted or unsubstituted C₆₋₃₀ aryl a substituted or unsubstituted C₂₋₃₀ heteroaryl containing at least one heteroatom of O and S, a fused ring of a substituted or unsubstituted C₃₋₁₂ aliphatic ring and a C₆₋₃₀ aryl or a substituted or unsubstituted C₆₋₃₀ arylsilyl, with the proviso that the case where all of R₁ to R₃ are unsubstituted phenyl is excluded; and an organic light emitting device comprising the same.

### CROSS-REFERENCE TO RELATED APPLICATION(S)

This application is a National Stage Application of International Application No. PCT/KR2023/015639 filed on Oct. 11, 2023, which claims priority from and the benefit of Korean Patent Application No. 10-2022-0129527 filed on Oct. 11, 2022 and Korean Patent Application No. 10-2023-0135250 filed on Oct. 11, 2023 in the Korean Intellectual Property Office, the disclosures of which are incorporated herein by reference in their entirety.

### TECHNICAL FIELD

The present disclosure relates to a novel compound and an organic light emitting device comprising the same.

### BACKGROUND ART

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer may be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continued need to develop a new material for the organic material used in the organic light emitting device as described above.

### PRIOR ART LITERATURE

Patent Literature 1: Korean Unexamined Patent Publication No. 10-2000-0051826

### DETAILED DESCRIPTION OF THE INVENTION

### Technical Problem

It is an object of the present disclosure to provide a novel organic light emitting material and an organic light emitting device comprising the same.

### Technical Solution

Provided herein is a compound represented by the following Chemical Formula 1:

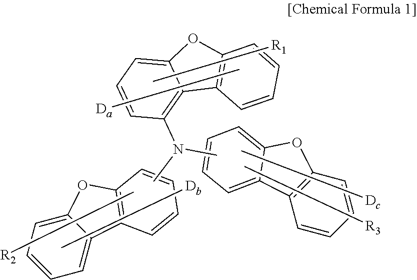

wherein, in Chemical Formula 1,
D is deuterium,
a to c are each independently an integer of 0 to 6,
R₁ to R₃ are each independently hydrogen; deuterium; or -Lₐ-Rₐ, with the proviso that at least one of R₁ to R₃ is -Lₐ-Rₐ,
Lₐ is a single bond; or a substituted or unsubstituted C₆₋₃₀ arylene,
Rₐ is a substituted or unsubstituted C₆₋₃₀ aryl; a substituted or unsubstituted C₂₋₃₀ heteroaryl containing at least one heteroatom of O and S; a fused ring of a substituted or unsubstituted C₃₋₁₂ aliphatic ring and a C₆₋₃₀ aryl; or a substituted or unsubstituted C₆₋₃₀ arylsilyl,
with the proviso that the case where all of R₁ to R₃ are unsubstituted phenyl is excluded.

Also provided herein is an organic light emitting device comprising: a first electrode; a second electrode that is provided opposite to the first electrode; and one or more organic material layers that are provided between the first electrode and the second electrode, wherein at least one layer of the organic material layers comprises the compound represented by Chemical Formula 1.

### Advantageous Effects

The compound represented by Chemical Formula 1 described above can be used as a material of an organic material layer in an organic light emitting device, and can improve the efficiency, achieve low driving voltage and/or improve lifetime characteristics in the organic light emitting device. In particular, the compound represented by Chemical Formula 1 can be used as a hole transport auxiliary material.

### BRIEF DESCRIPTION OF THE DRAWINGS

FIG. 1 shows an example of an organic light emitting device comprising a substrate 1, an anode 2, an organic material layer 3, and a cathode 4.

FIG. 2 shows an example of an organic light emitting device comprising a substrate 1, an anode 2, a hole injection layer 5, a hole transport layer 6, a hole transport auxiliary layer 7, a light emitting layer 8, a hole blocking layer 9, an electron transport layer 10, an electron injection layer 11, and a cathode 4.

FIG. 3 shows an example of an organic light emitting device comprising a substrate 1, an anode 2, a hole injection layer 5, a hole transport layer 6, a hole transport auxiliary layer 7, a light emitting layer 8, a hole blocking layer 9, an electron injection and transport layer 12, and cathode 4.

### DETAILED DESCRIPTION OF THE EMBODIMENTS

Hereinafter, embodiments of the present disclosure will be described to help understanding of the disclosed subject matter.

The present disclosure provides the compound represented by Chemical Formula 1.

In the present disclosure, the notation

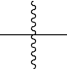

or [symbol] means a bond linked to another substituent group.

In the present disclosure, the term “substituted or unsubstituted” means being unsubstituted or substituted with one or more substituents selected from the group consisting of deuterium; a halogen group; a nitrile group; a nitro group; a hydroxy group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a heterocyclic group containing at least one of N, O and S atoms, or being unsubstituted or substituted with a substituent to which two or more substituents of the above-exemplified substituents are linked. For example, “a substituent in which two or more substituents are linked” may be a biphenyl group. Namely, a biphenyl group may be an aryl group, or it may be interpreted as a substituent formed by linking two phenyl groups.

In one example, the term “substituted or unsubstituted” may be understood as meaning “being unsubstituted or substituted with one or more substituents selected from the group consisting of deuterium, halogen, cyano, a C₁₋₁₀ alkyl, a C₁₋₁₀ alkoxy, and a C₆₋₂₀ aryl. Also, the term “substituted with one or more substituents” as used herein may be understood as meaning “being substituted with one to the maximum number of substitutable hydrogens”.

In the present disclosure, the carbon number of a carbonyl group is not particularly limited, but is preferably 1 to 40. Specifically, it may be a group having the following structure, but is not limited thereto.

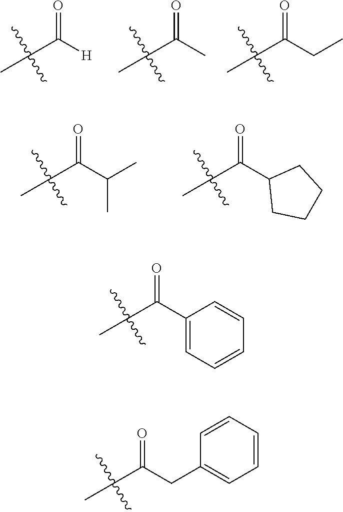

In the present disclosure, an ester group may have a structure in which oxygen of the ester group may be substituted by a straight-chain, branched-chain, or cyclic alkyl group having 1 to 25 carbon atoms, or an aryl group having 6 to 25 carbon atoms. Specifically, the ester group may be a group having the following structural formulas, but is not limited thereto.

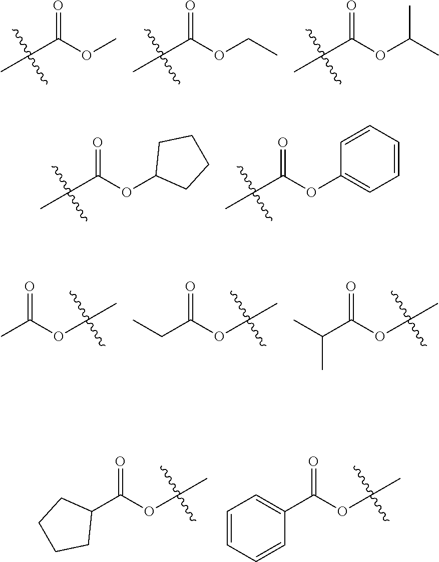

In the present disclosure, the carbon number of an imide group is not particularly limited, but is preferably 1 to 25. Specifically, the imide group may be a group having the following structural formulas, but is not limited thereto.

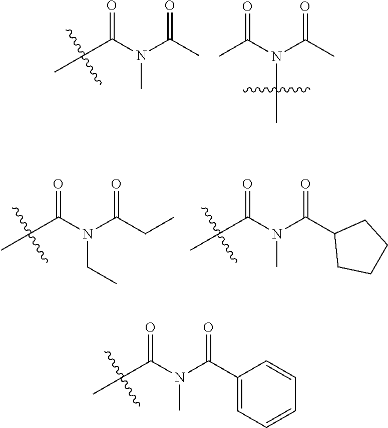

In the present disclosure, specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group may be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group may be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, of the cycloalkyl group is not particularly limited, but the carbon number thereof is preferably 3 to 60. According to another embodiment, the carbon number of the cycloalkyl group is 3 to 30. According to another embodiment, the carbon number of the cycloalkyl group is 3 to 20. According to another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it may be a monocyclic aryl group or a polycyclic aryl group. According to one embodiment, the carbon number of the aryl group is 6 to 30. According to one embodiment, the carbon number of the aryl group is 6 to 20. The aryl group may be a phenyl group, a biphenyl group, a terphenyl group or the like as the monocyclic aryl group, but is not limited thereto. The polycyclic aryl group includes a naphthyl group, an anthracenyl group, a phenanthrenyl group, a pyrenyl group, a perylenyl group, a chrysenyl group, a fluorenyl group, or the like, but is not limited thereto.

In the present disclosure, the fluorenyl group may be substituted, and two substituent groups may be linked with each other to form a spiro structure. In the case where the fluorenyl group is substituted.

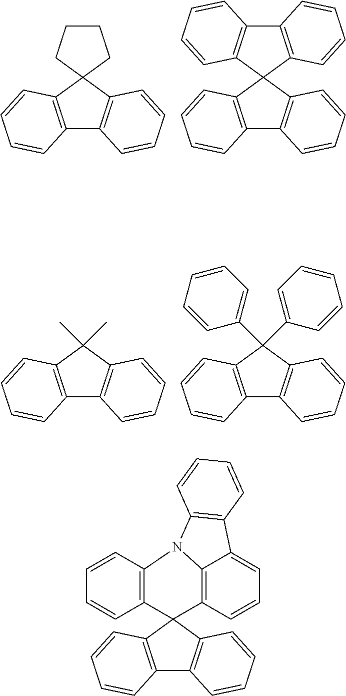

and the like can be formed. However, the structure is not limited thereto.

In the present disclosure, a heterocyclic group is a heterocyclic group containing one or more of O, N, Si and S as a heteroatom, and the carbon number thereof is not particularly limited, but is preferably 2 to 60. Examples of the heterocyclic group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group and the arylamine group is the same as the examples of the aryl group as defined above. In the present disclosure, the alkyl group in the aralkyl group, the alkylaryl group and the alkylamine group is the same as the examples of the alkyl group as defined above. In the present disclosure, the heteroaryl in the heteroarylamine can be applied to the description of the heterocyclic group as defined above. In the present disclosure, the alkenyl group in the aralkenyl group is the same as the examples of the alkenyl group as defined above. In the present disclosure, the description of the aryl group as defined above may be applied except that the arylene is a divalent group. In the present disclosure, the description of the heterocyclic group as defined above can be applied except that the heteroarylene is a divalent group. In the present disclosure, the description of the aryl group or cycloalkyl group as defined above can be applied except that the hydrocarbon ring is not a monovalent group but formed by combining two substituent groups. In the present disclosure, the description of the heterocyclic group as defined above can be applied, except that the heterocyclic group is not a monovalent group but formed by combining two substituent groups.

In the present disclosure, the term “deuterated or substituted with deuterium” means that at least one of the substitutable hydrogens in a compound, a divalent linking group, or a monovalent substituent has been substituted with deuterium.

Further, the term “unsubstituted or substituted with deuterium” or “substituted or unsubstituted with deuterium” means that “mono to the maximum number of unsubstituted or substitutable hydrogens have been substituted with deuterium.” In one example, the term “phenanthryl unsubstituted or substituted with deuterium” may be understood as meaning “phenanthryl unsubstituted or substituted with 1 to 9 deuterium atoms”, considering that the maximum number of hydrogen that can be substituted with deuterium in the phenanthryl structure is 9.

Further, “deuterated structure” means to include compounds, divalent linking groups, or monovalent substituents of all structures in which at least one hydrogen is substituted with deuterium. As an example, the deuterated structure of phenyl can be understood to refer to monovalent substituents of all structures in which at least one substitutable hydrogen in the phenyl group is substituted with deuterium, as follows.

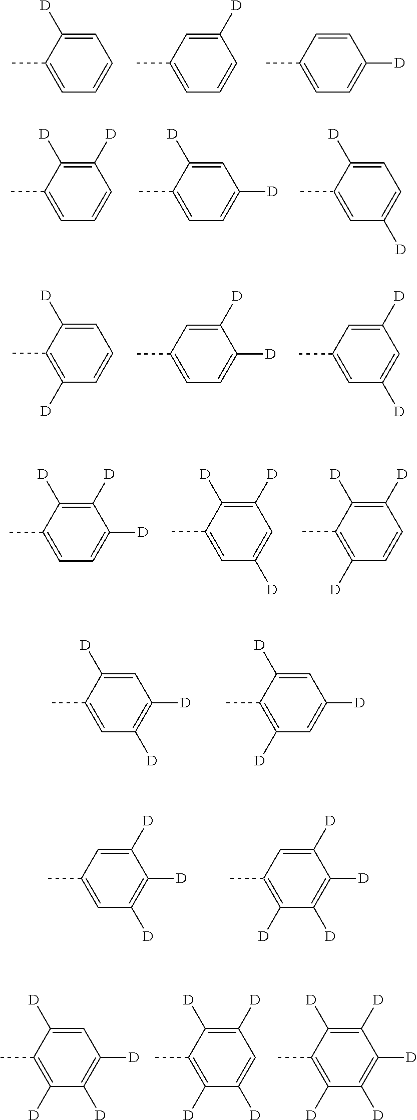

In addition, the “deuterium substitution rate” or “degree of deuteration” of a compound means that the ratio of the number of substituted deuterium atoms to the total number of hydrogen atoms (the sum of the number of hydrogen atoms substitutable with deuterium and the number of substituted deuterium atoms in a compound) that can exist in the compound is calculated as a percentage. Therefore, when the “deuterium substitution rate” or “degree of deuteration” of a compound is “K %”, it means that K % of the hydrogen atoms substitutable with deuterium in the compound are substituted with deuterium.

At this time, the “deuterium substitution rate” or “degree of deuteration” can be determined according to a commonly known method using MALDI-TOF MS (Matrix-Assisted Laser Desorption/Ionization Time-of-Flight Mass Spectrometer), a nuclear magnetic resonance spectroscopy (¹H NMR), TLC/MS (Thin-Layer Chromatography/Mass Spectrometry), GC/MS (Gas Chromatography/Mass Spectrometry), or the like. More specifically, when using MALDI-TOF MS, the “deuterium substitution rate” or “degree of deuteration” may be obtained by determining the number of substituted deuterium in the compound through MALDI-TOF MS analysis, and then calculating the ratio of the number of substituted deuterium to the total number of hydrogen atoms that can exist in the compound as a percentage.

The compound of Chemical Formula 1 is an amine to which three dibenzofurans are bonded, wherein at least one of the three dibenzofurans is bonded to an amine via carbon 1, and at least one of the three dibenzofurans has a substituent represented by -Lₐ-Rₐ.

As such a structure is satisfied, the compound of Chemical Formula 1 can be used in an organic material layer, particularly a light emitting layer, of an organic light emitting device, and thus exhibit low voltage, high efficiency, and/or long lifetime characteristics.

In the compound of Chemical Formula 1, at least one hydrogen of the three dibenzofurans may be substituted with deuterium. Also, at least one hydrogen of the substituent represented by -Lₐ-Rₐ may be substituted with deuterium.

Preferably, Lₐ is a single bond; phenylene; biphenyldiyl; or naphthalenediyl.

In one embodiment, the Rₐ is a C₆₋₃₀ aryl; a C₂₋₃₀ heteroaryl containing at least one heteroatom of O and S; a fused ring of a C₃₋₁₂ aliphatic ring and a C₆₋₃₀ aryl; or a C₆₋₃₀ arylsilyl.

The C₆₋₃₀ aryl; the C₂₋₃₀ heteroaryl containing at least one heteroatom of O and S; the fused ring of a C₃₋₁₂ aliphatic ring and a C₆₋₃₀ aryl; or the C₆₋₃₀ arylsilyl may be unsubstituted, or substituted with at least one deuterium and/or at least one C₁₋₁₀ alkyl.

Preferably, Rₐ may be phenyl; biphenylyl; terphenylyl; naphthyl; (phenyl)naphthyl; phenanthrenyl; fluoranthenyl; benzo[a]phenanthrenyl; benzo[c]phenanthrenyl; dibenzofuran; dibenzothiophene; triphenylsilyl; 2,3-dihydro-1H-indenyl; 1,2,3,4-tetrahydronaphthalenyl; or 6,7,8,9-tetrahydro-5H-benzo[7]annulene, wherein the Rₐ may be unsubstituted or substituted with at least one substituent selected from the group consisting of deuterium; methyl; ethyl; propyl; isopropyl; and t-butyl.

Preferably, Rₐ may be phenyl; biphenylyl; terphenylyl; naphthyl; (phenyl)naphthyl; phenanthrenyl; benzo[a]phenanthrenyl; benzo[c]phenanthrenyl; triphenylenyl; 1,1,3,3-tetramethyl-2,3-dihydro-1H-indenyl; 1,2,3,4-tetrahydronaphthalenyl; 1,1,4,4-tetramethyl-1,2,3,4-tetrahydronaphthalenyl; or 6,7,8,9-tetrahydro-5H-benzo[7]annulene.

The compound represented by Chemical Formula 1 may not contain deuterium, or may contain at least one deuterium.

When the compound represented by Chemical Formula 1 contains deuterium, the deuterium substitution rate of the compound may be 1% to 100%. Specifically, the deuterium substitution rate of the compound may be 4% or more, 10% or more, 15% or more, 20% or more, 25% or more, 30% or more, 35% or more, or 40% or more, and 100% or less, 97% or less, 95% or less, 90% or less, or 89% or less.

In one example, the compound may not contain deuterium, or may contain 1 to 50 deuteriums. More specifically, the compound may not contain deuterium, or may contain 1 or more, 3 or more, 5 or more, 8 or more, or 10 or more, and 45 or less, 40 or less, 38 or less, 35 or less, or 33 or less deuteriums.

Representative examples of the compound represented by Chemical Formula 1 are as follows:

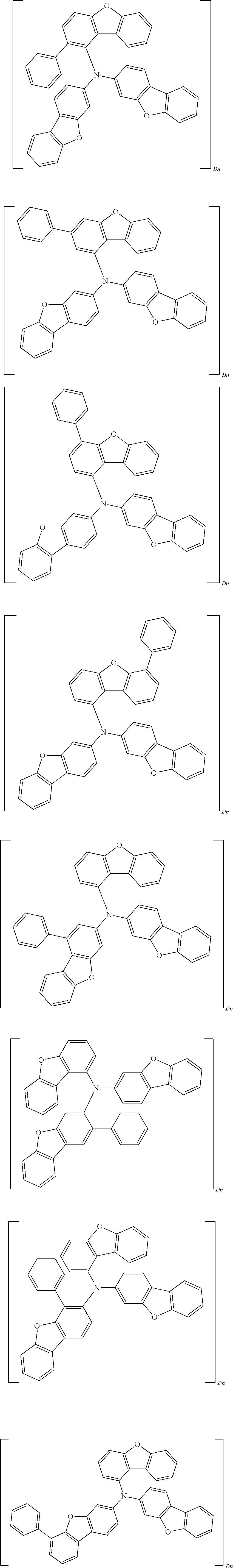

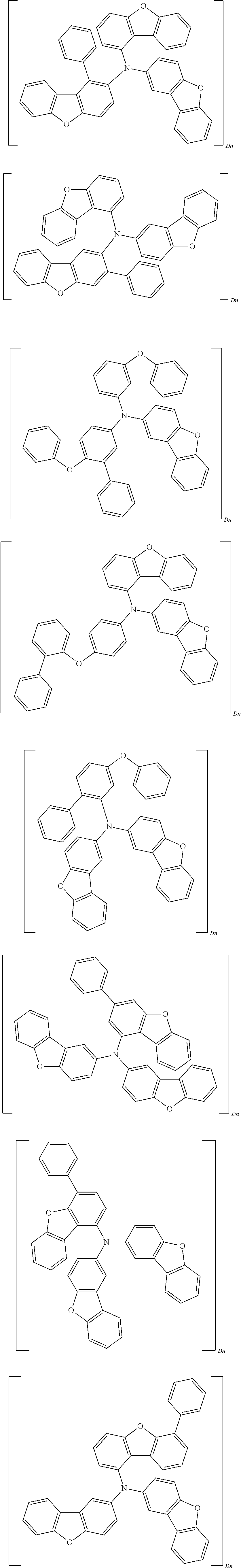

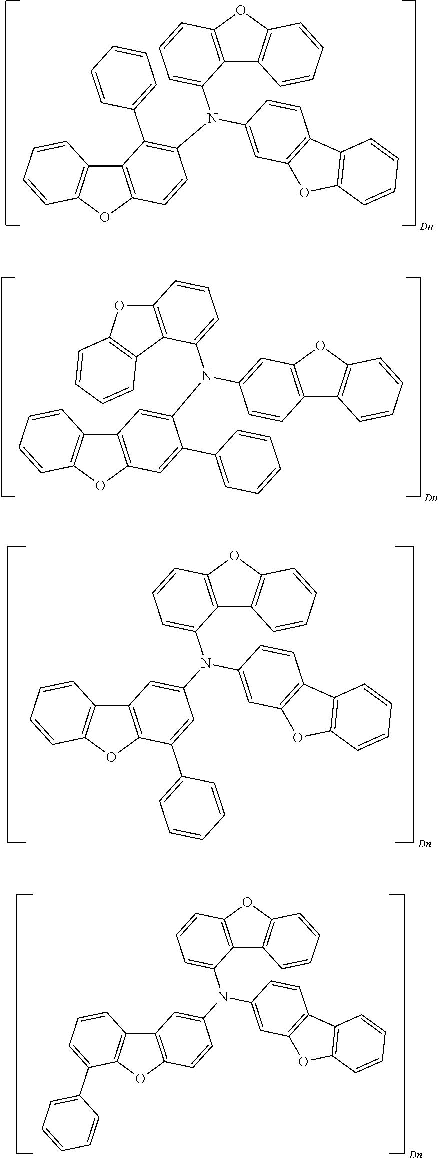

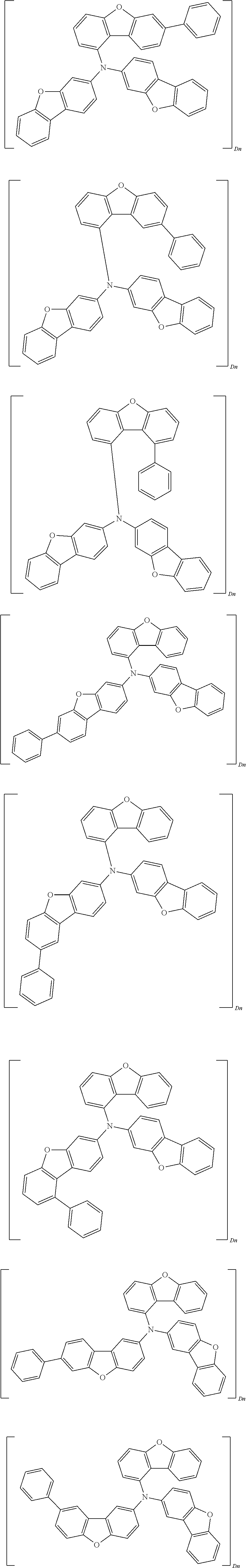

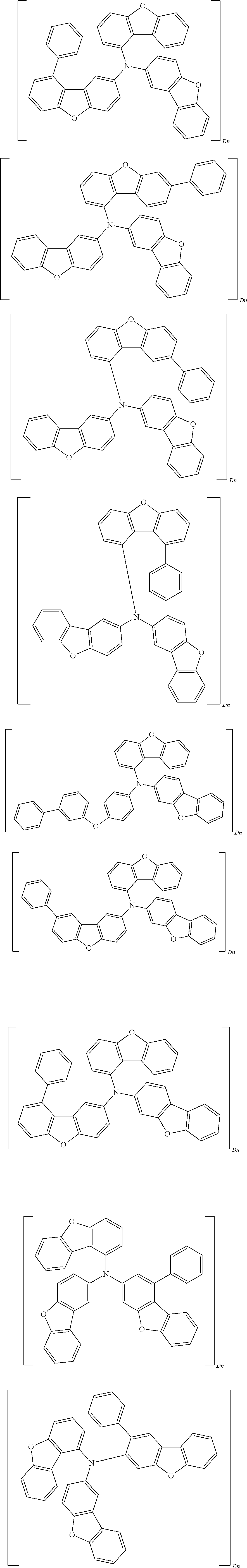

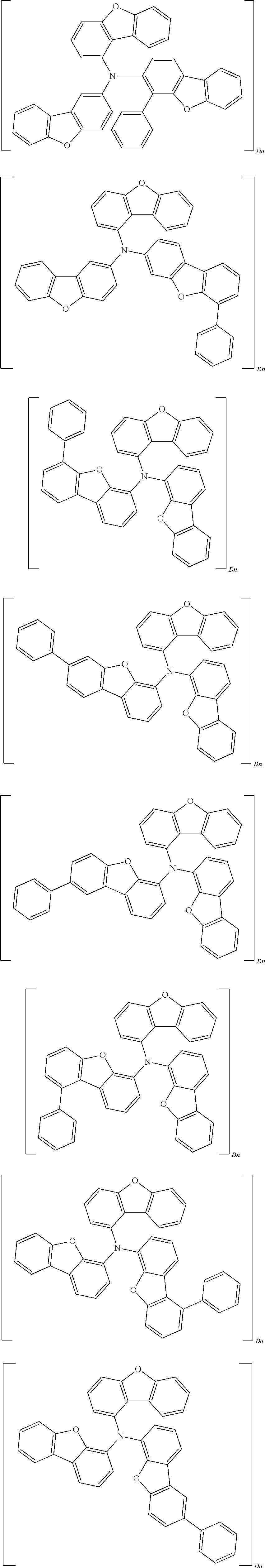

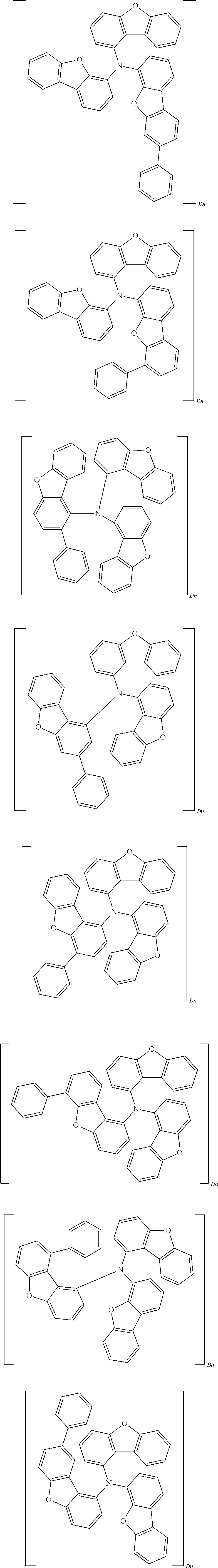

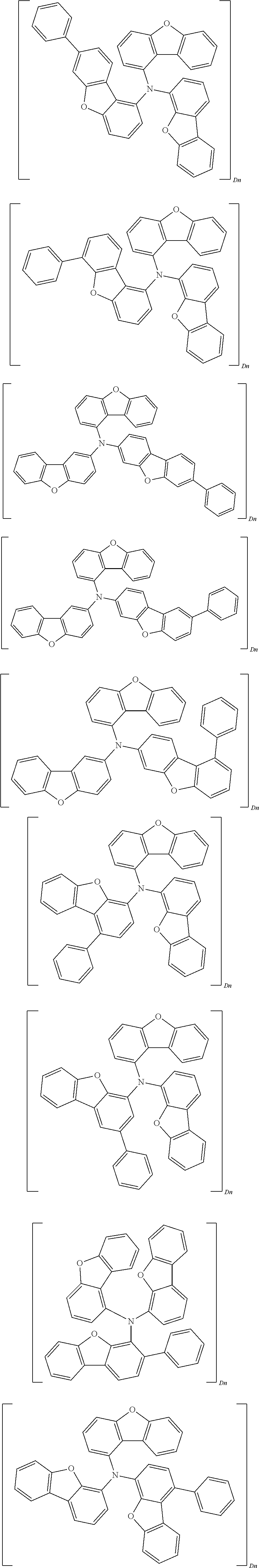

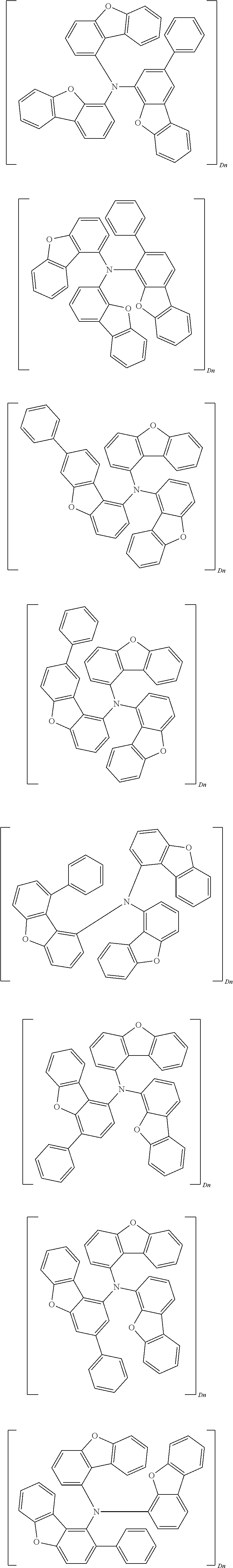

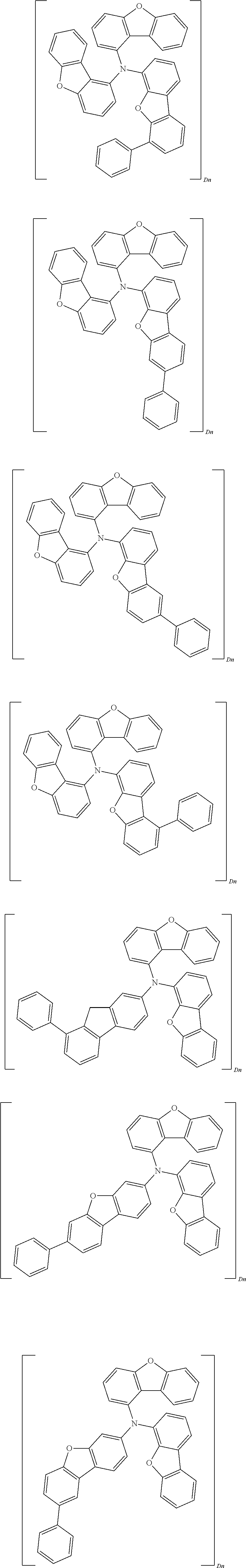

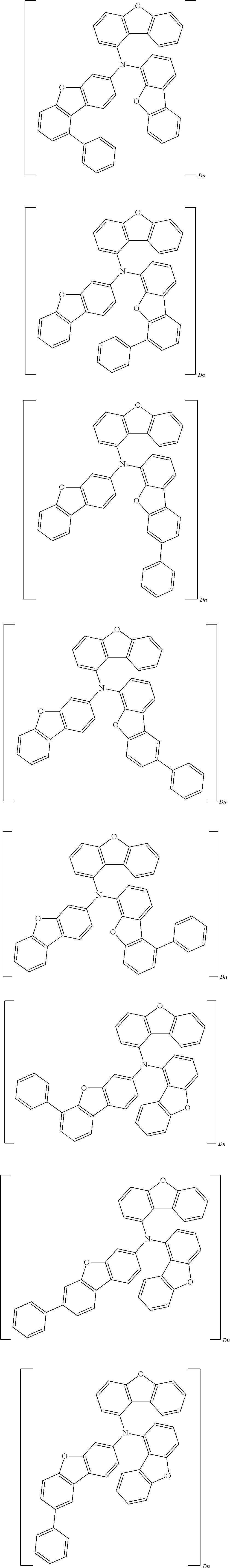

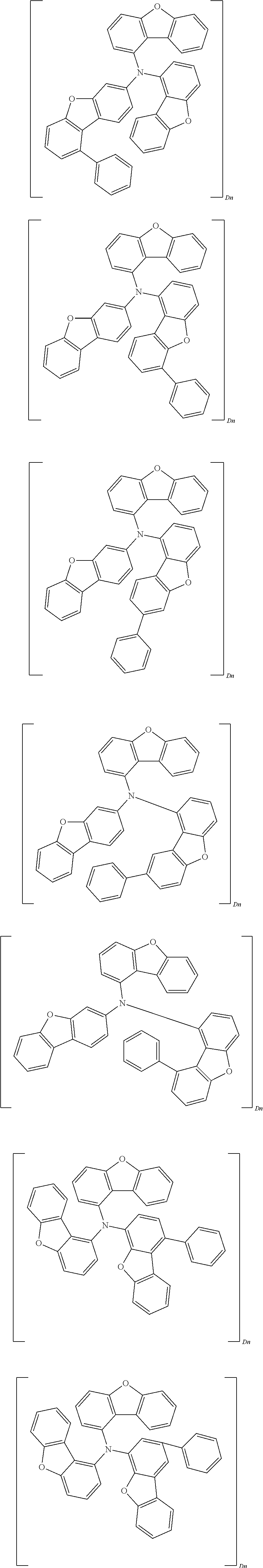

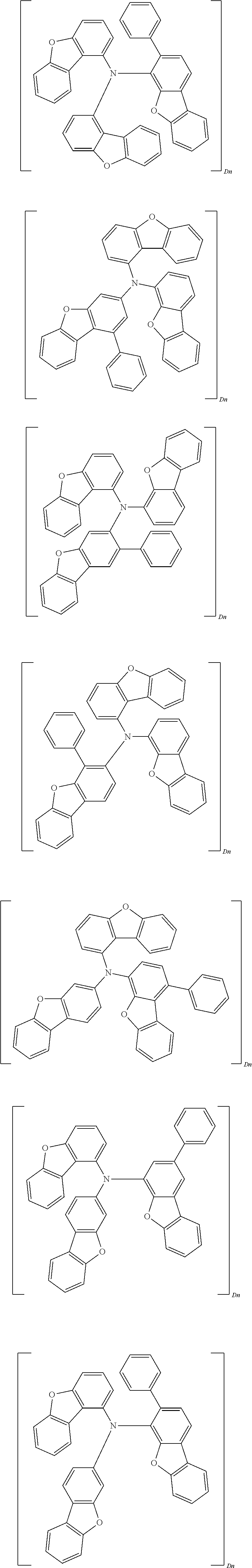

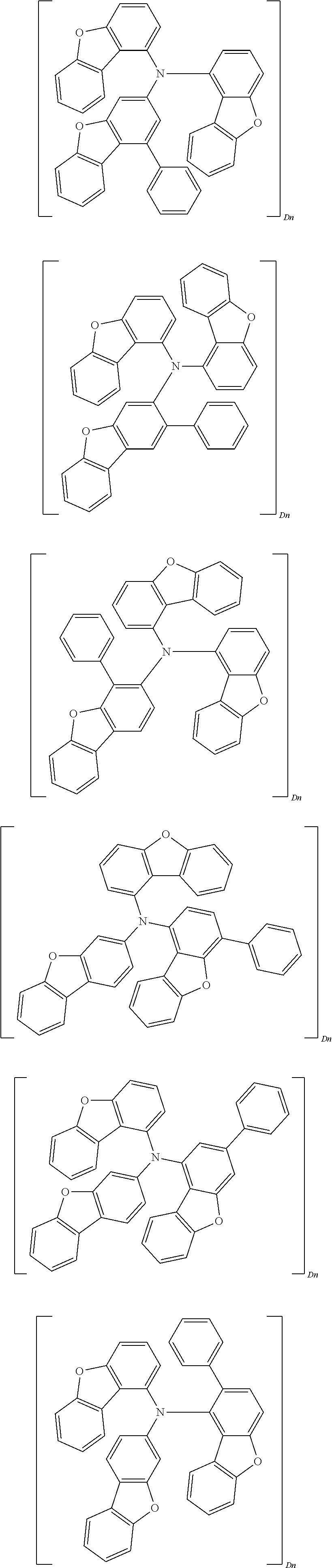

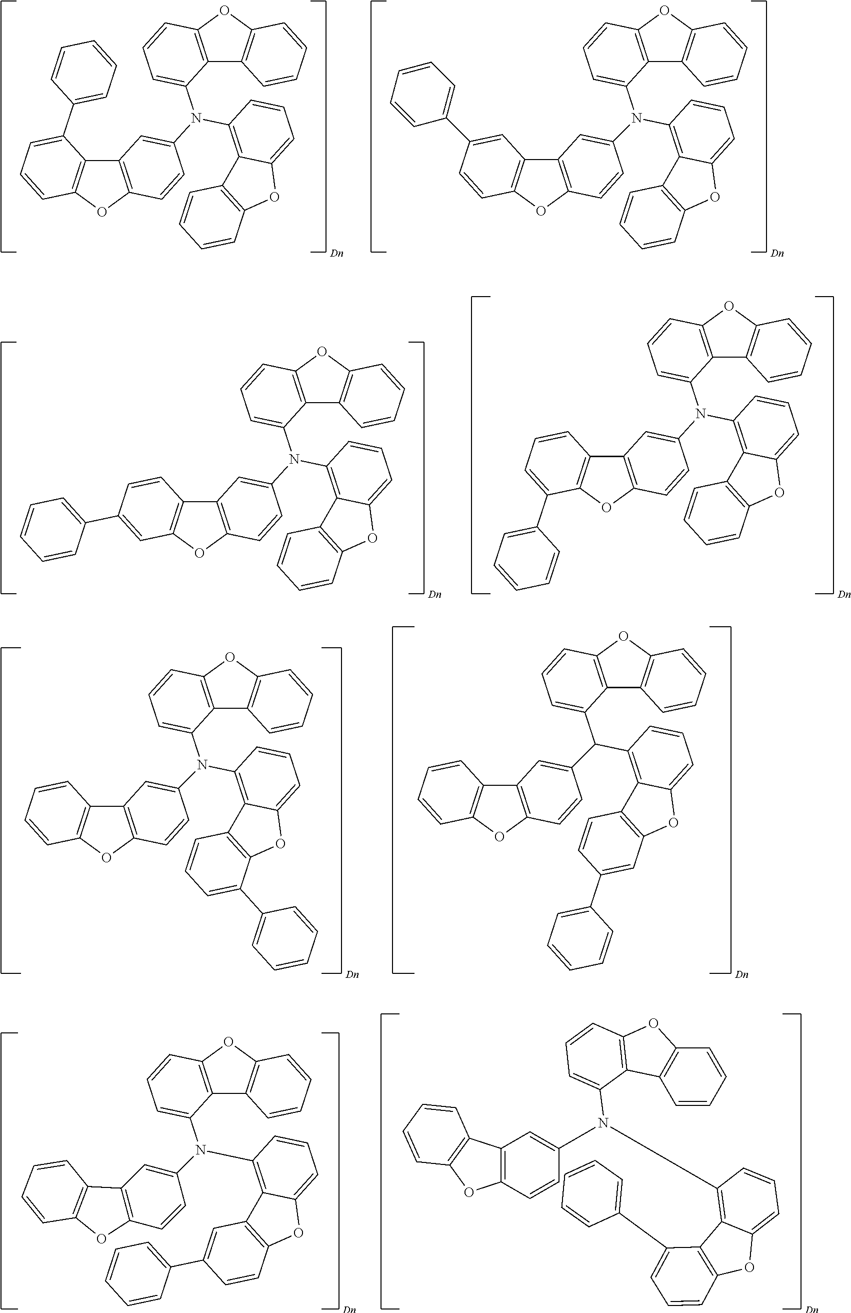

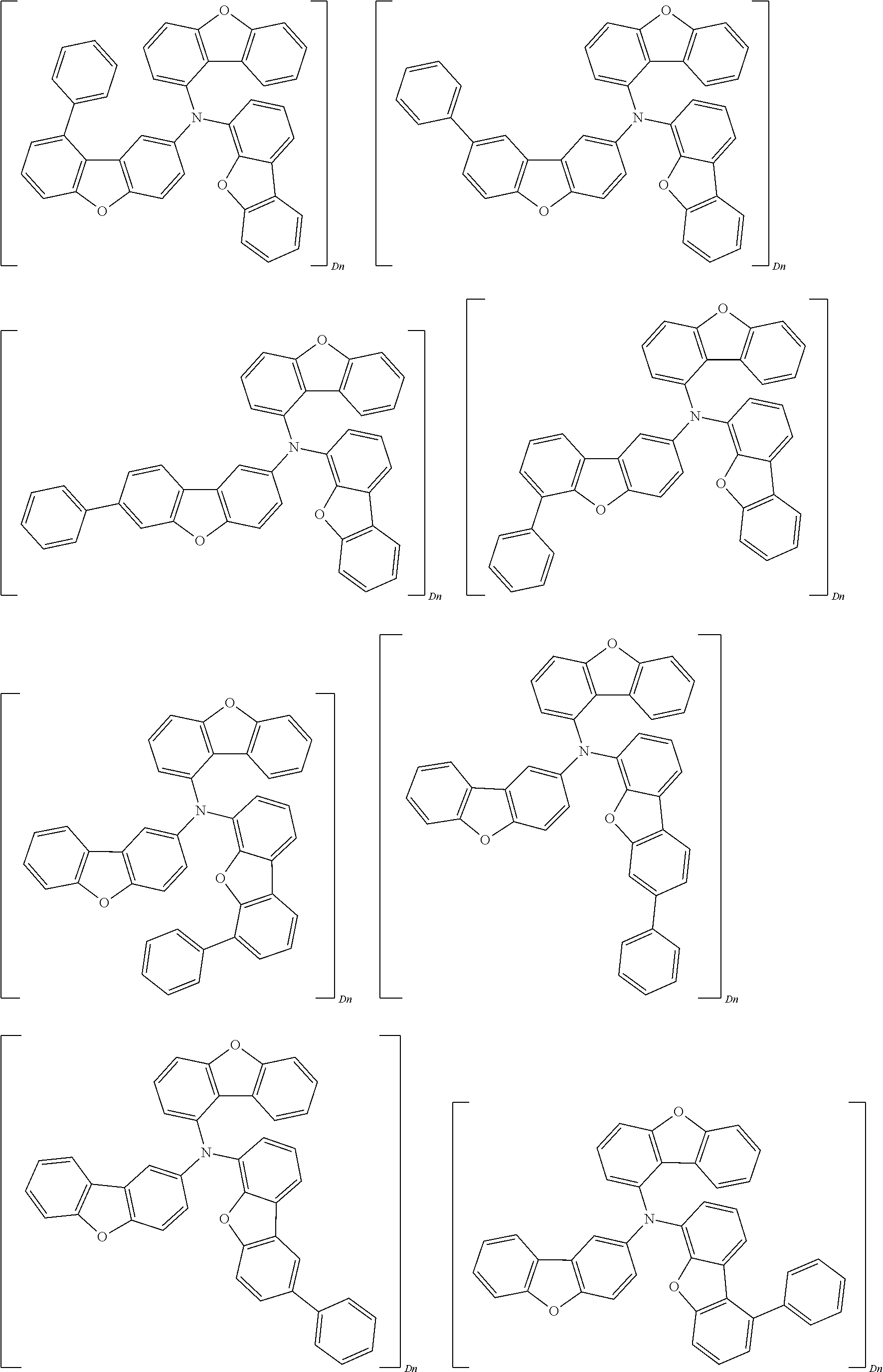

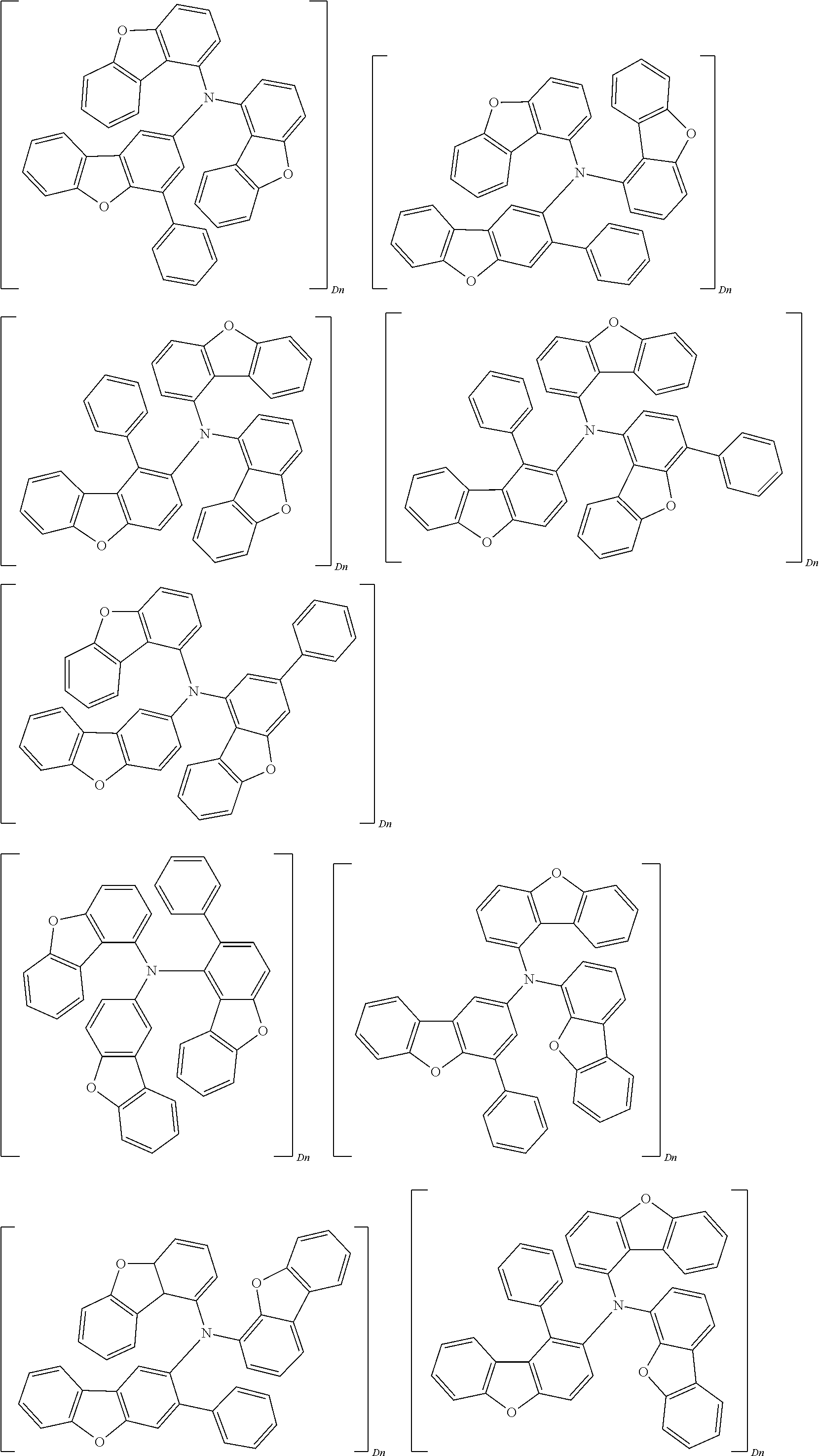

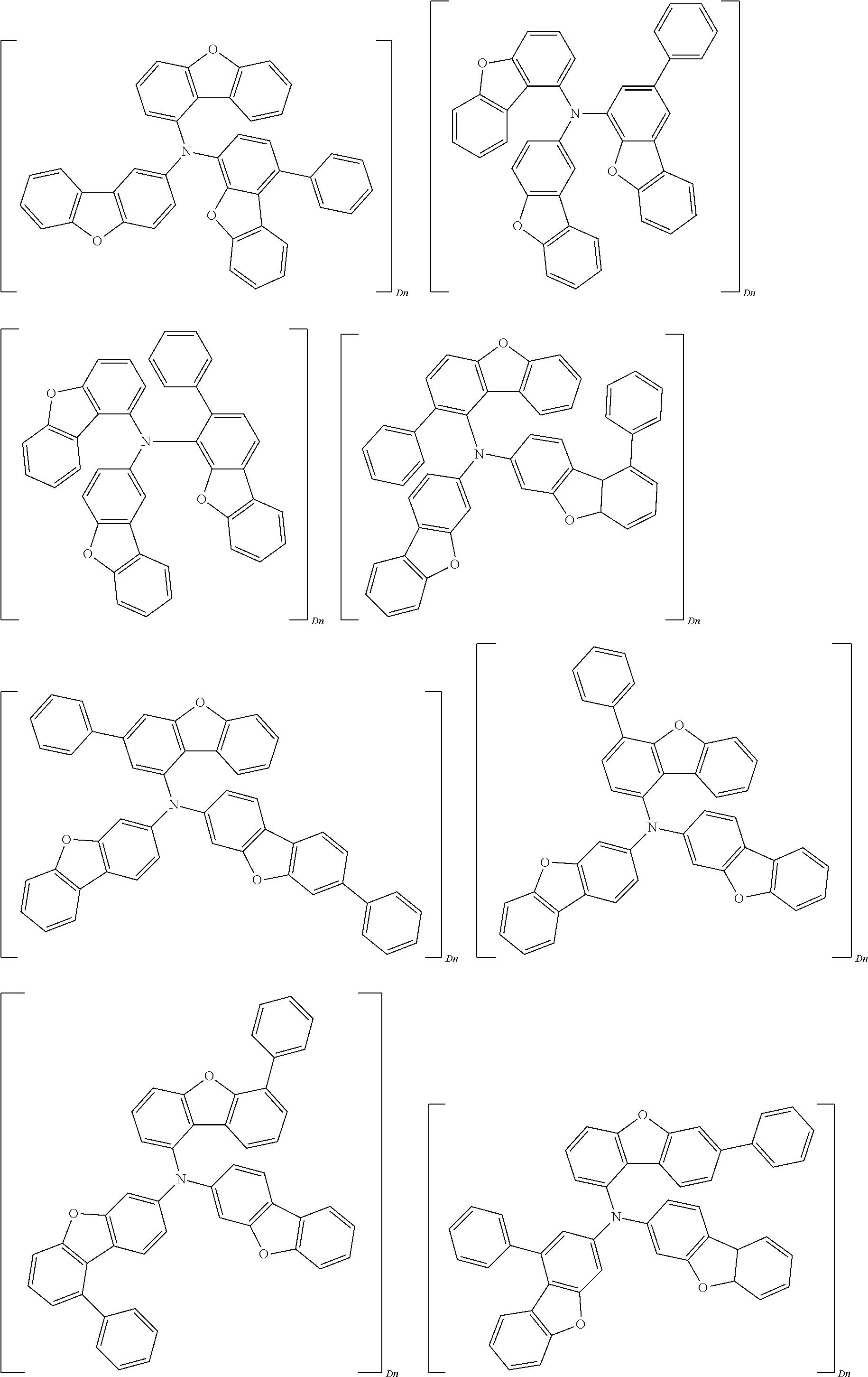

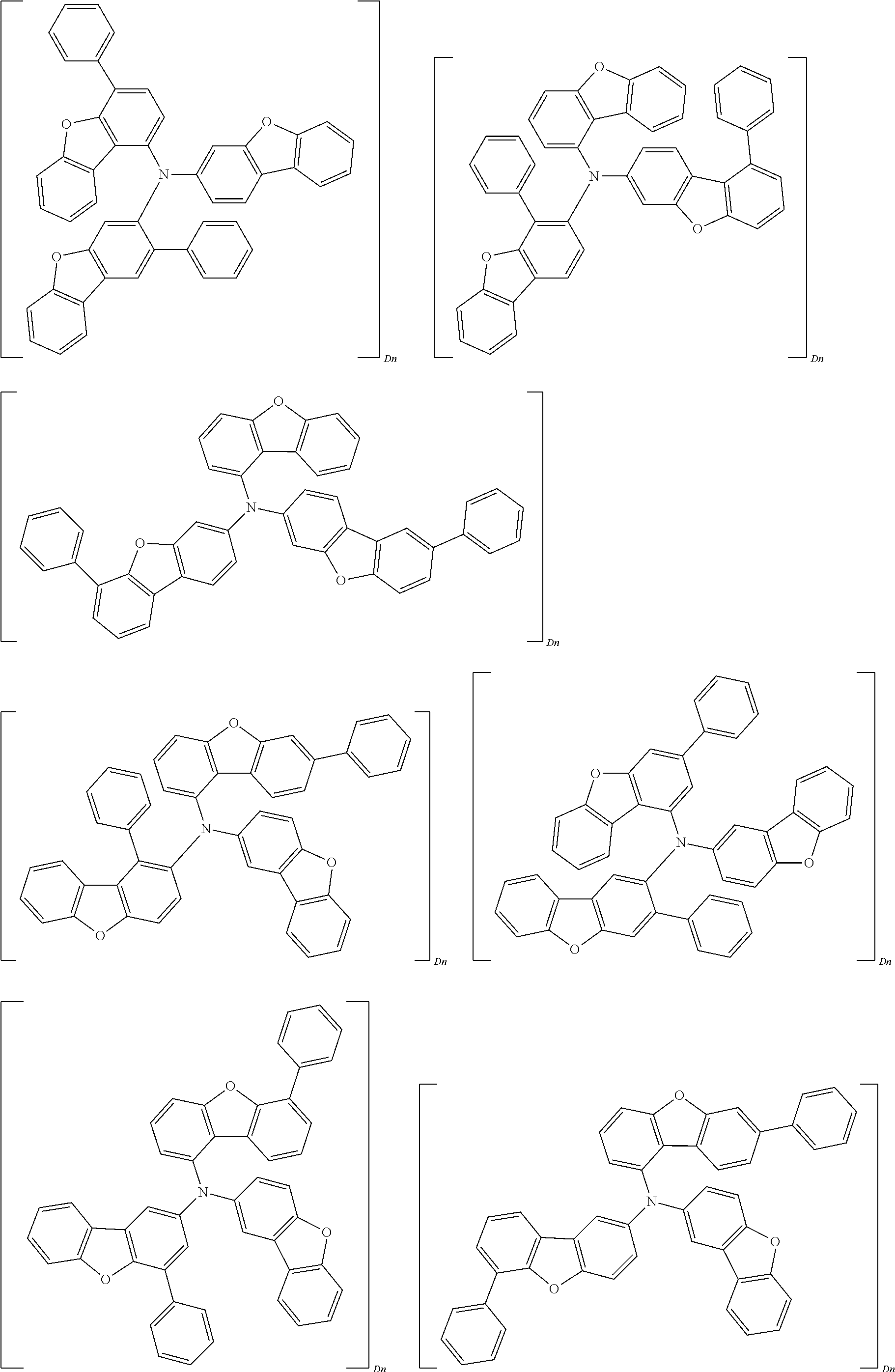

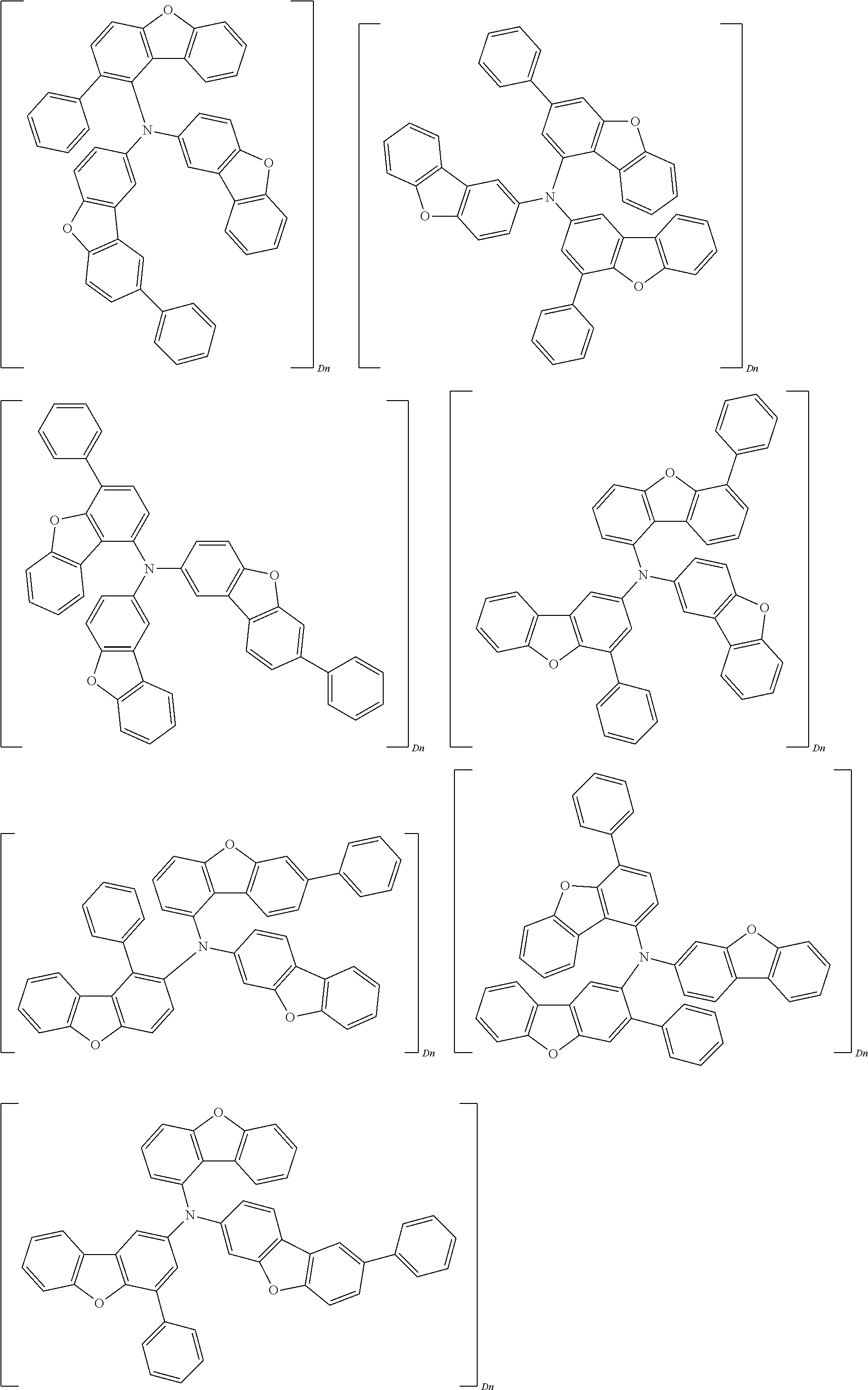

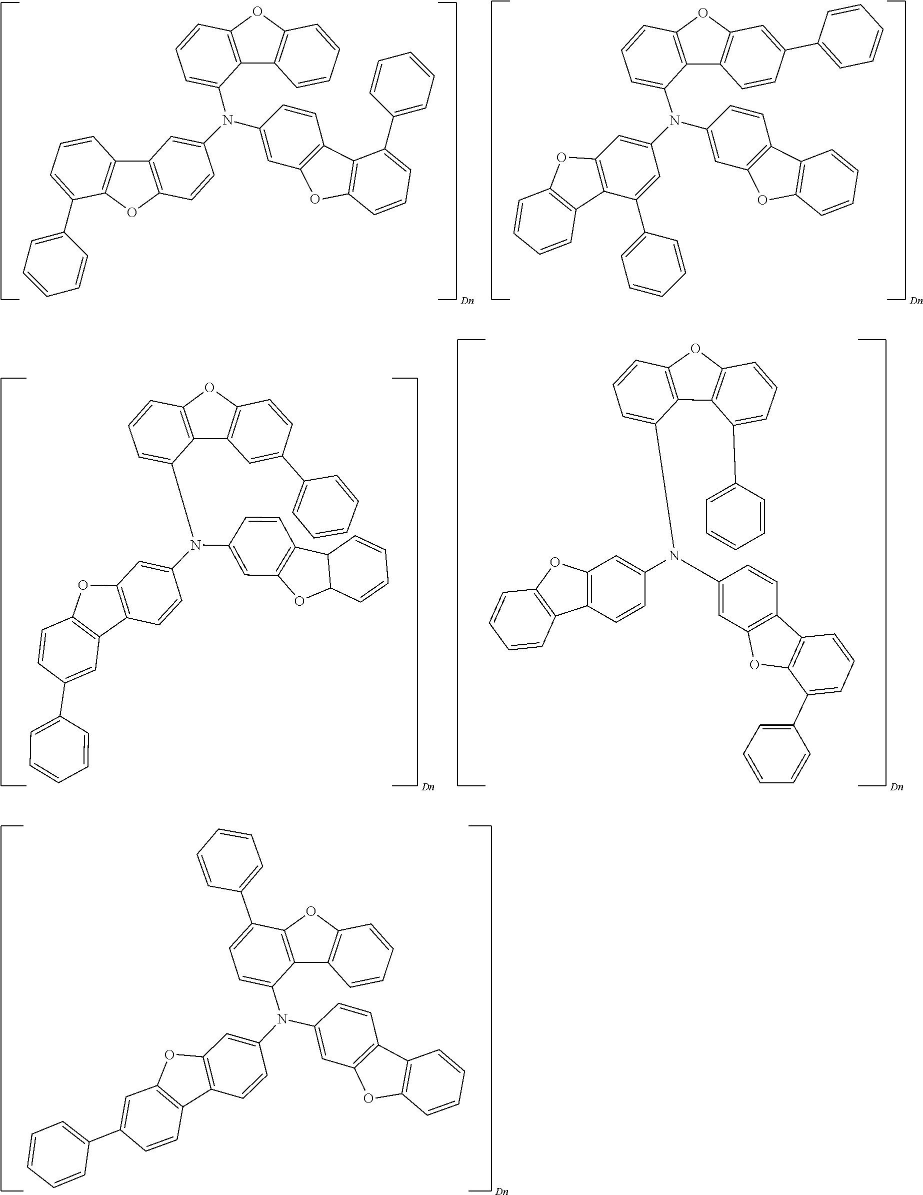

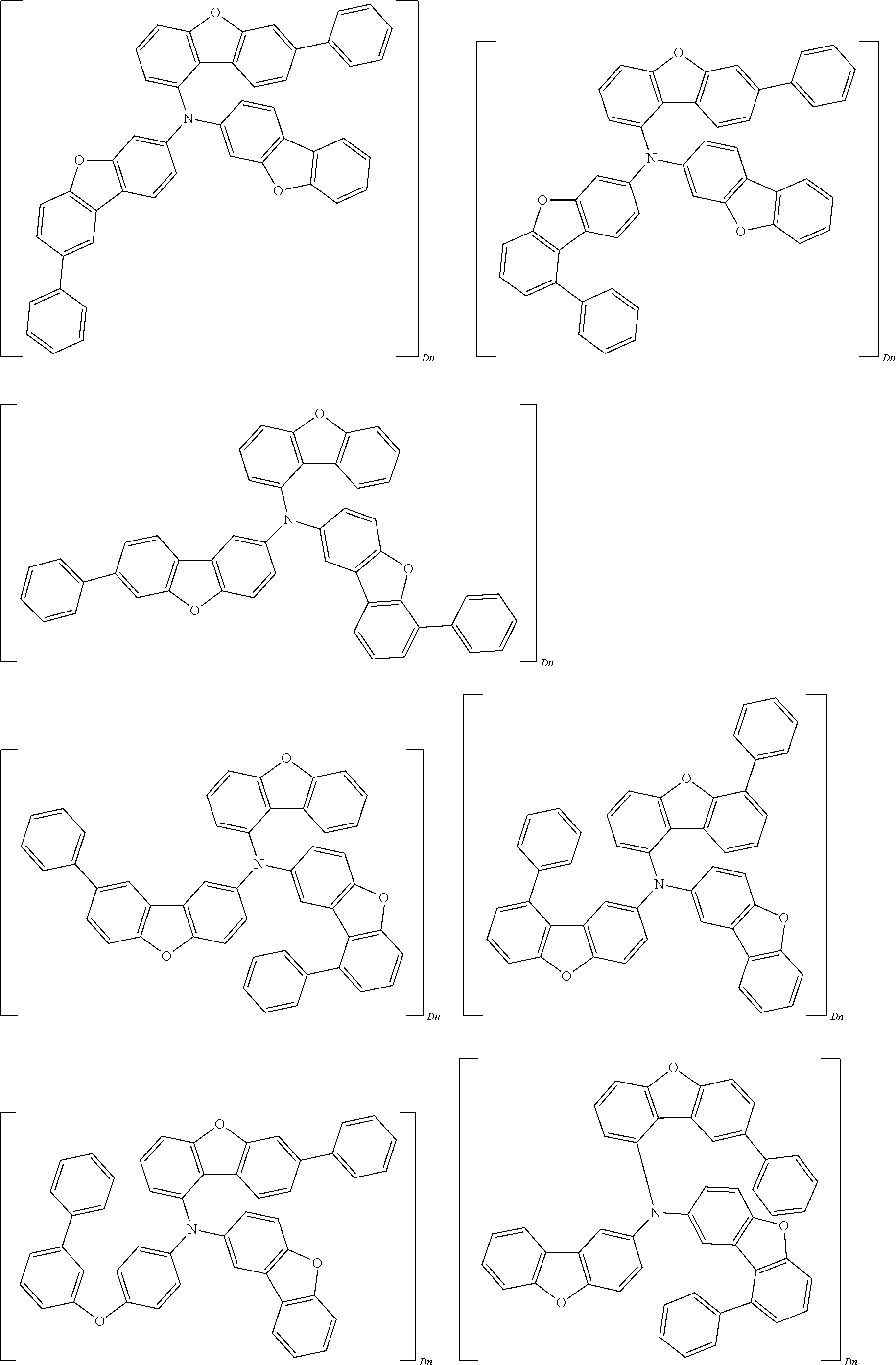

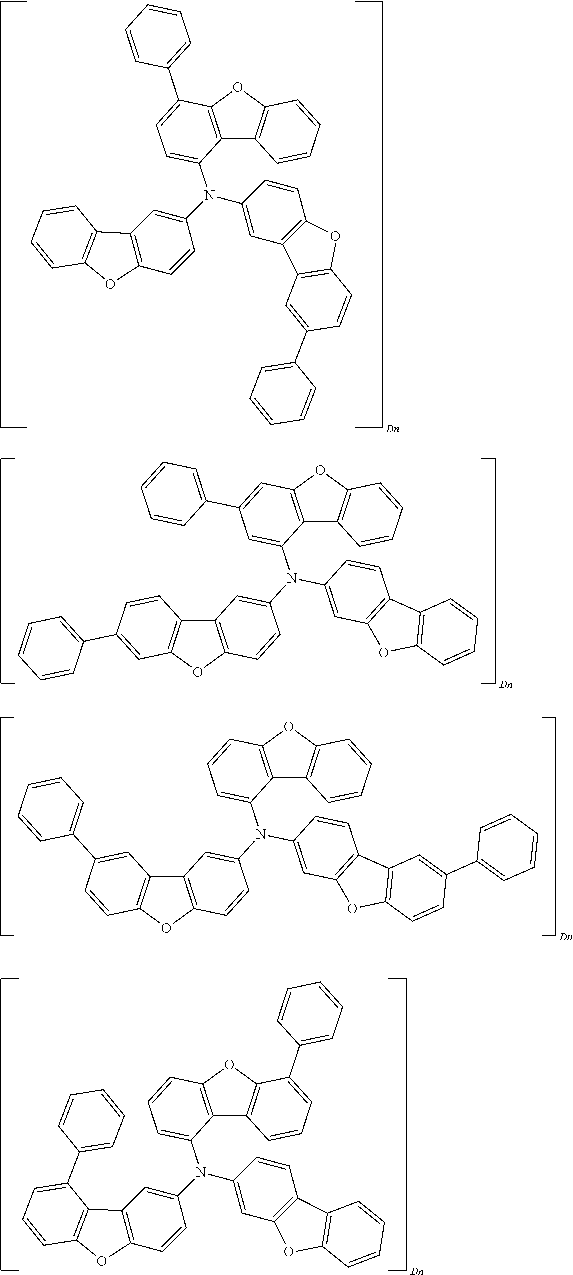

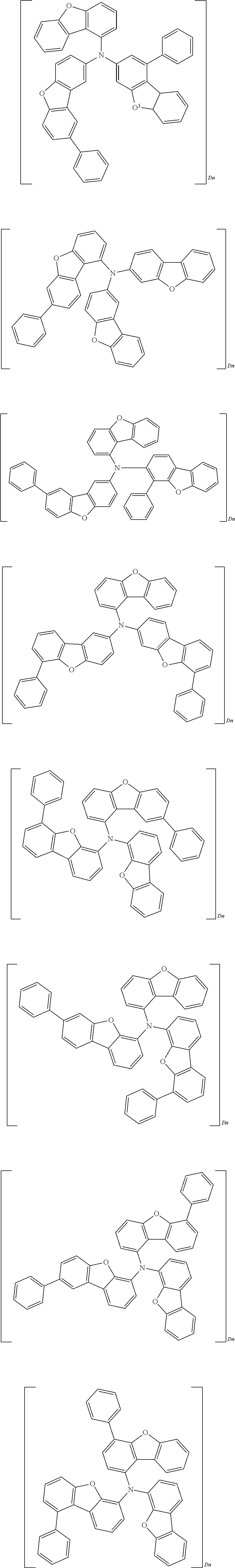

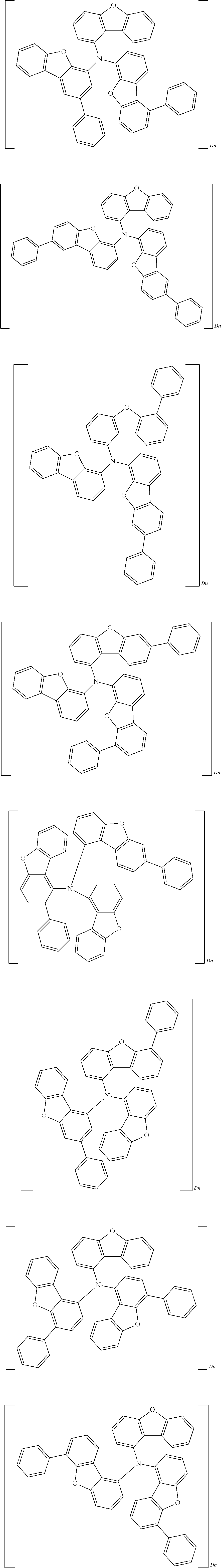

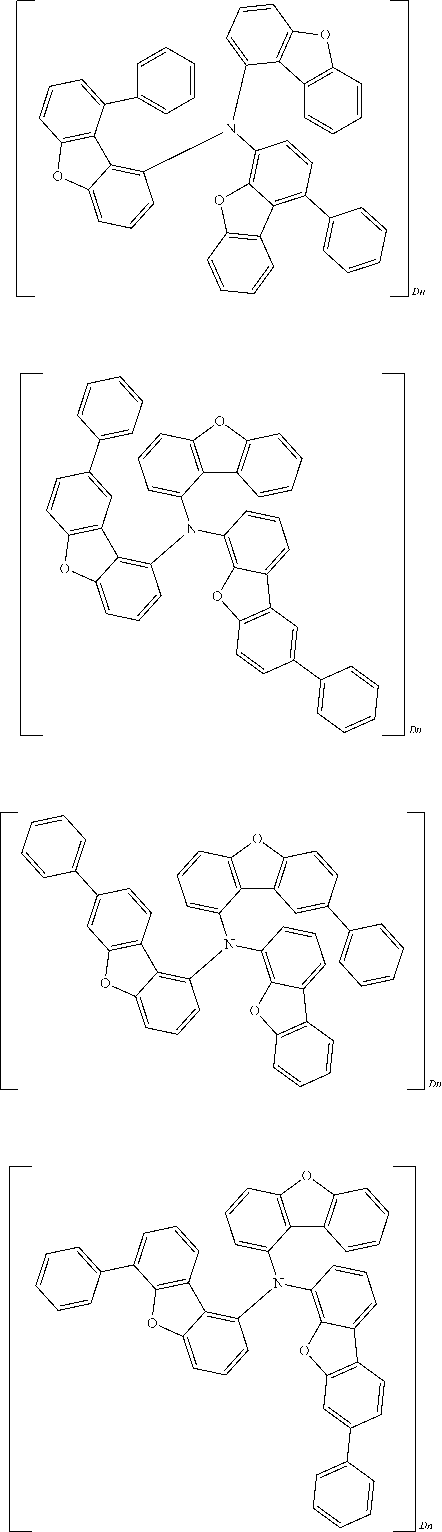

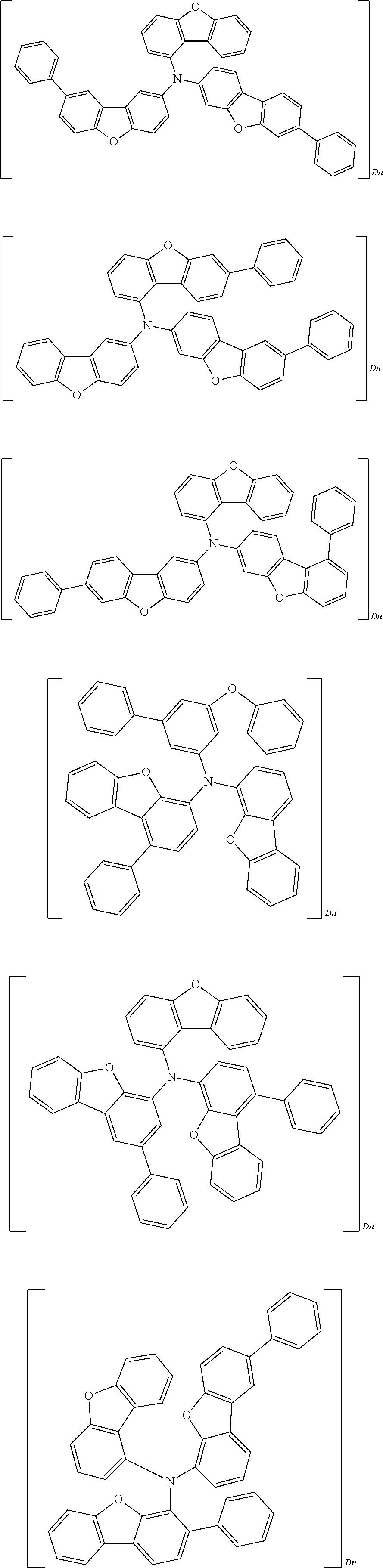

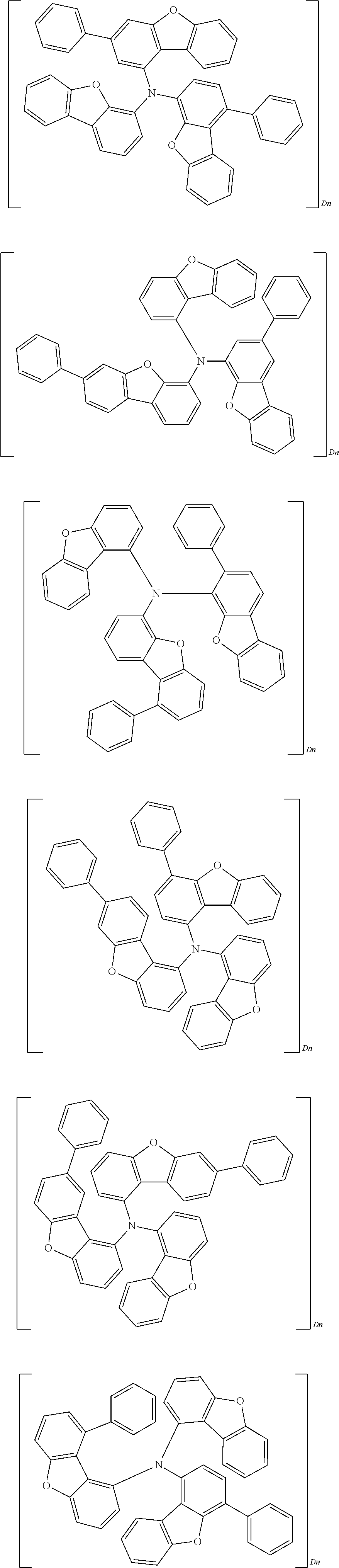

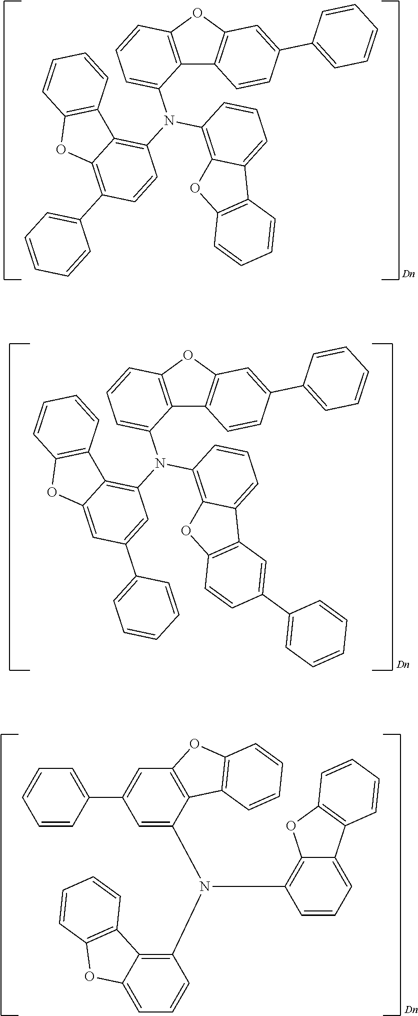

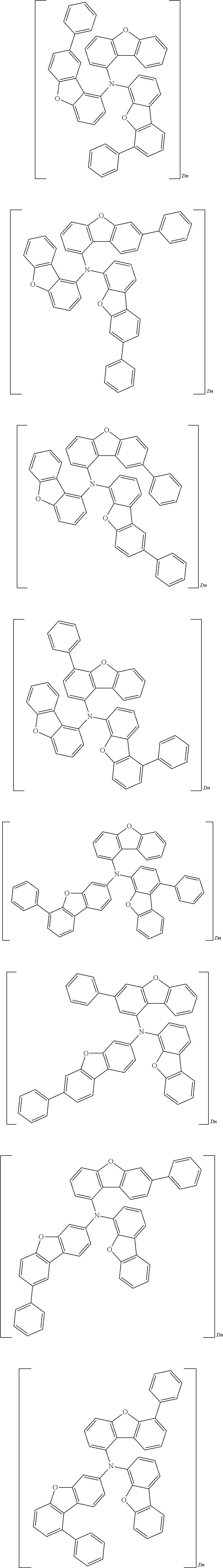

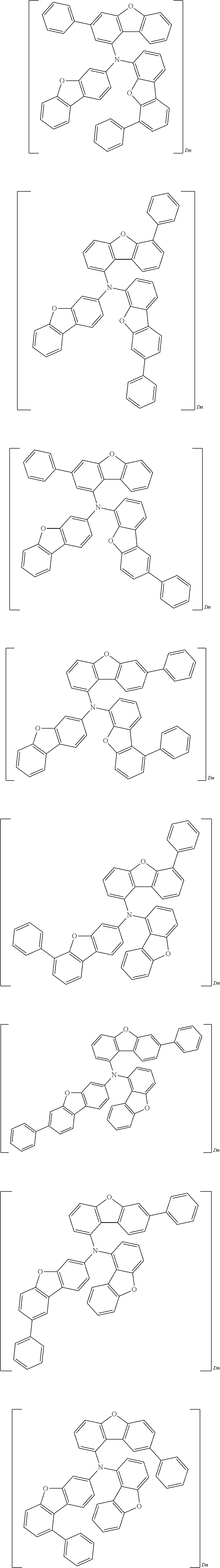

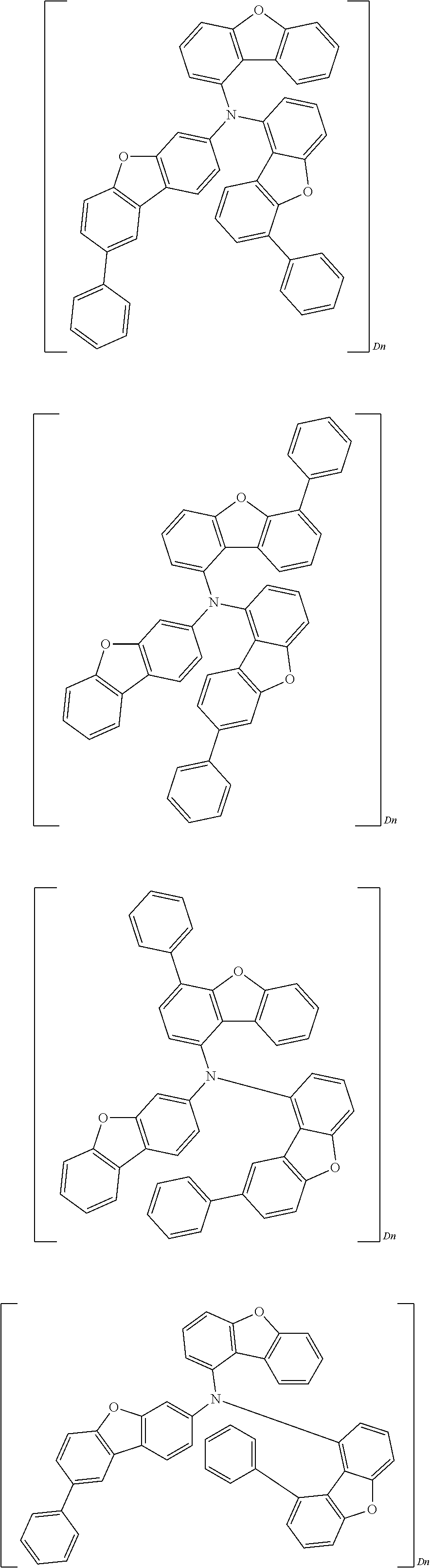

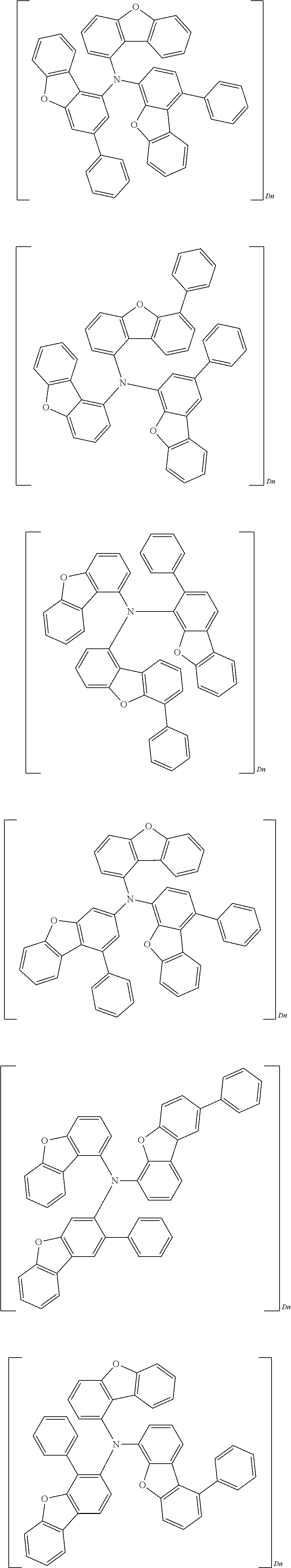

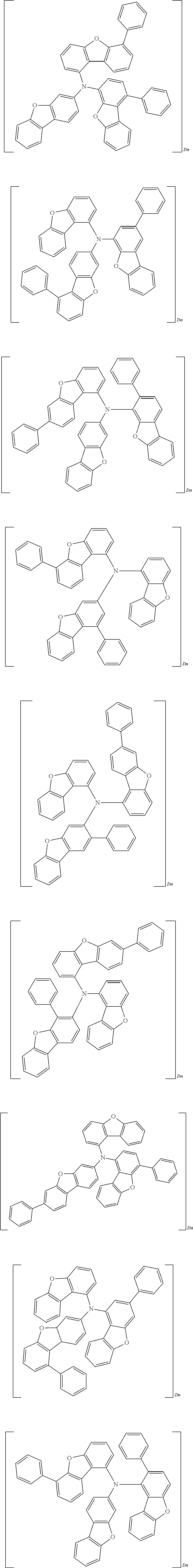

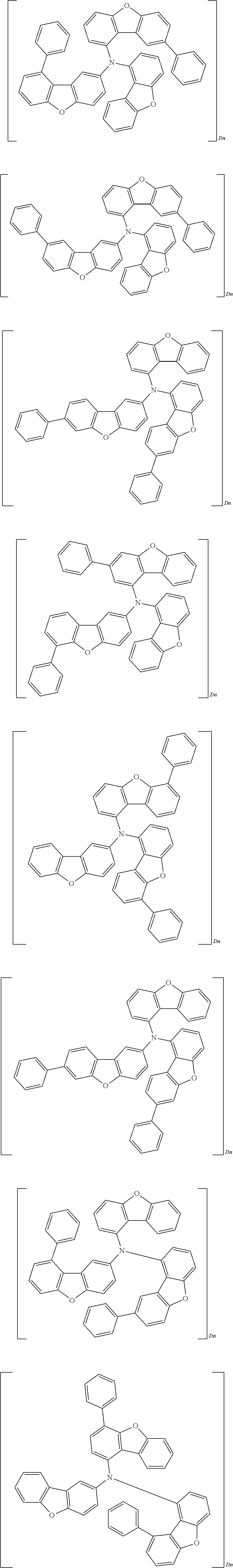

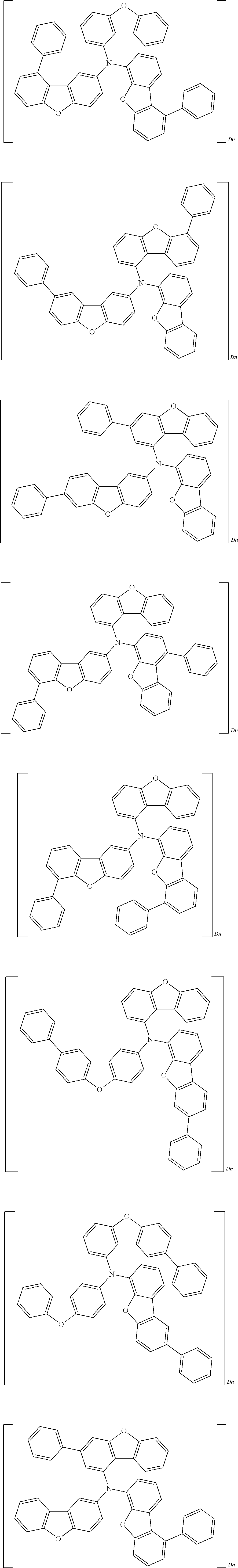

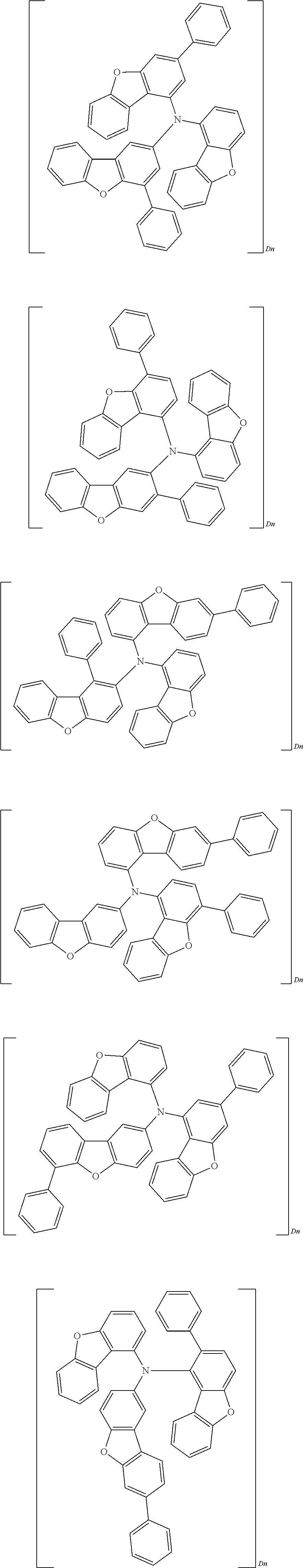

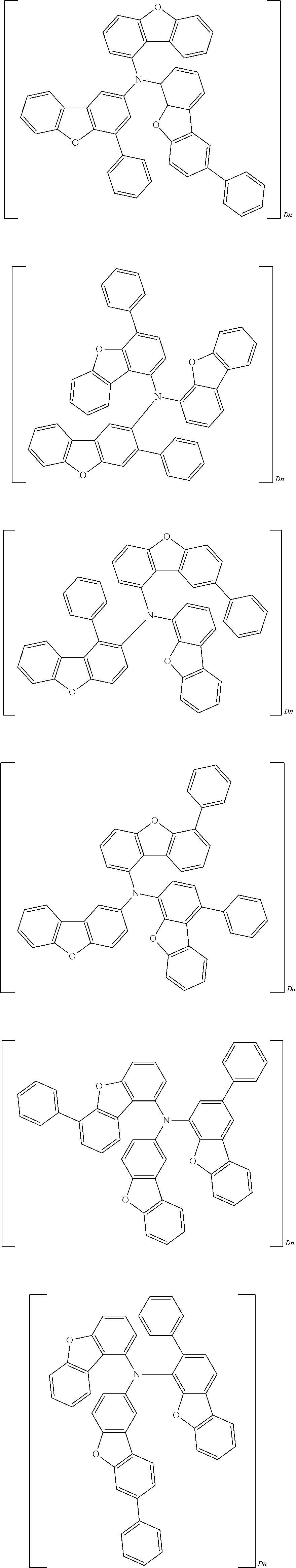

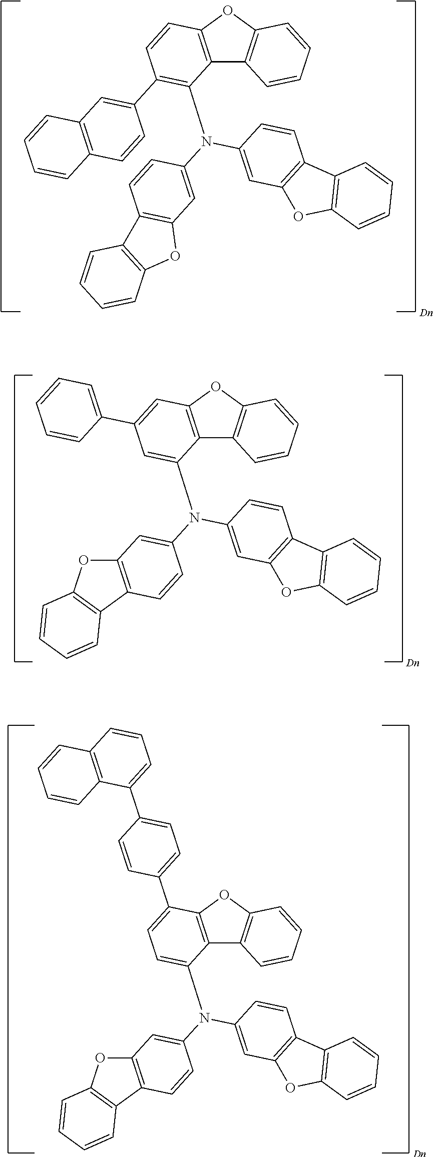

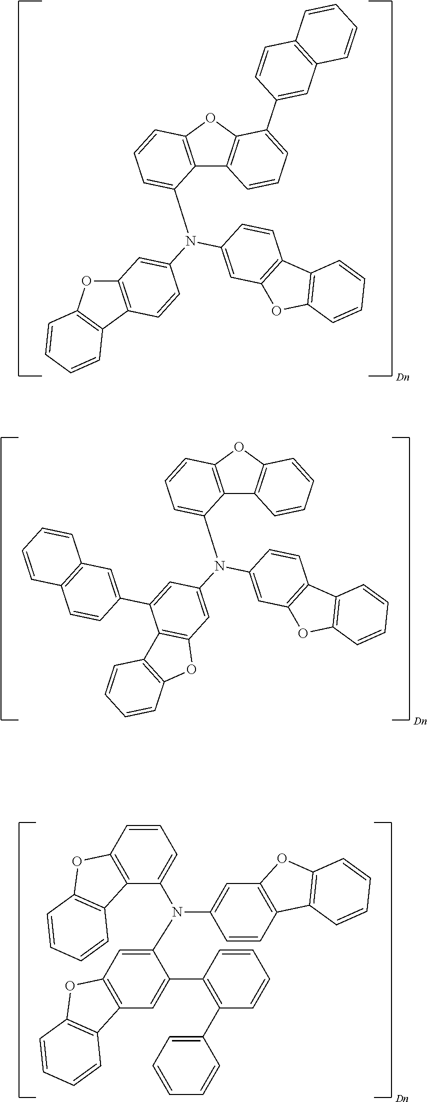

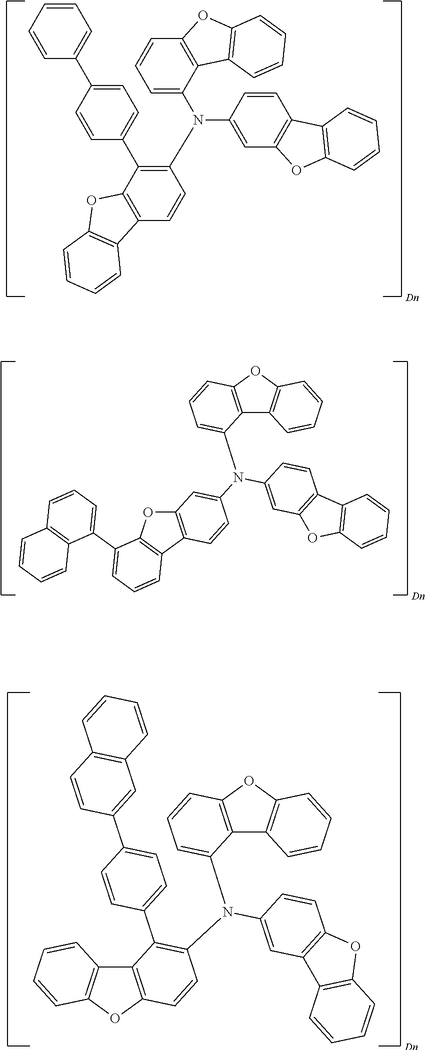

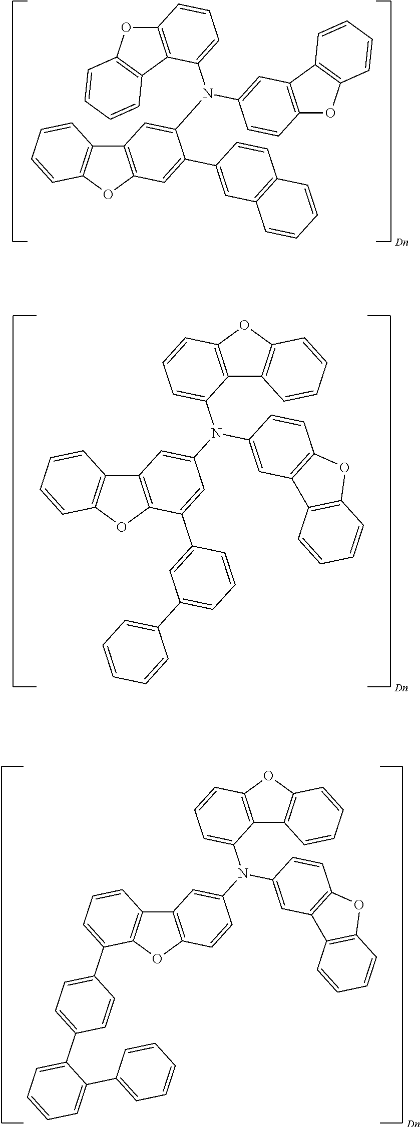

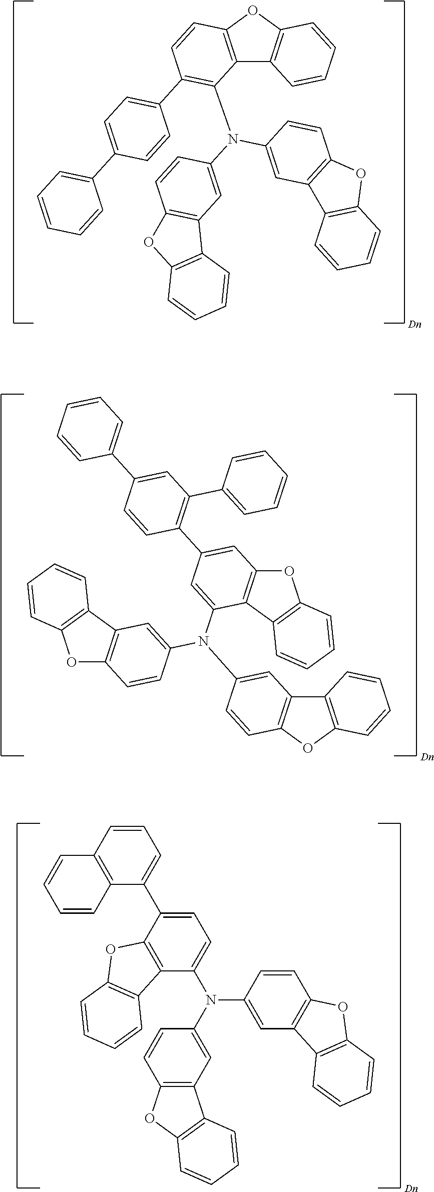

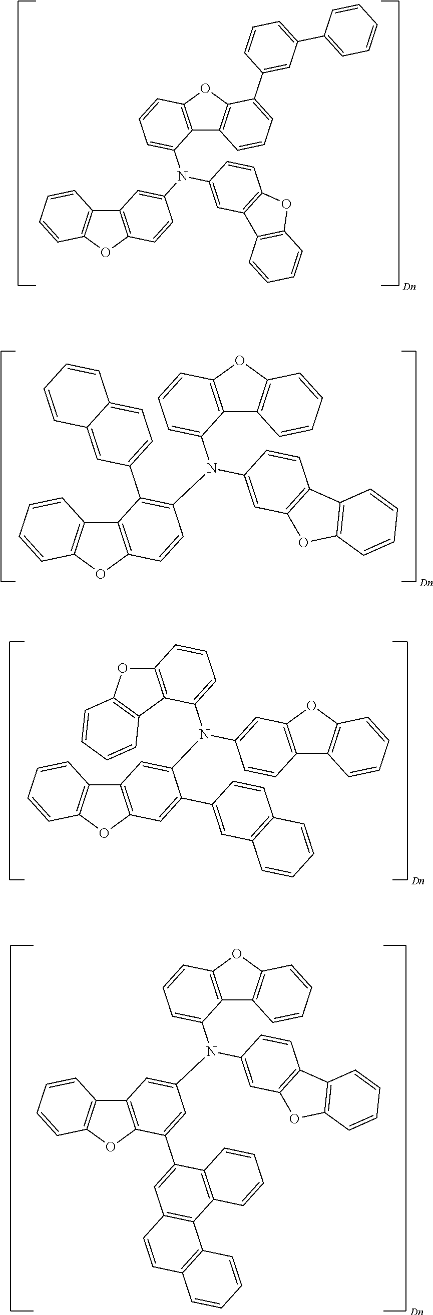

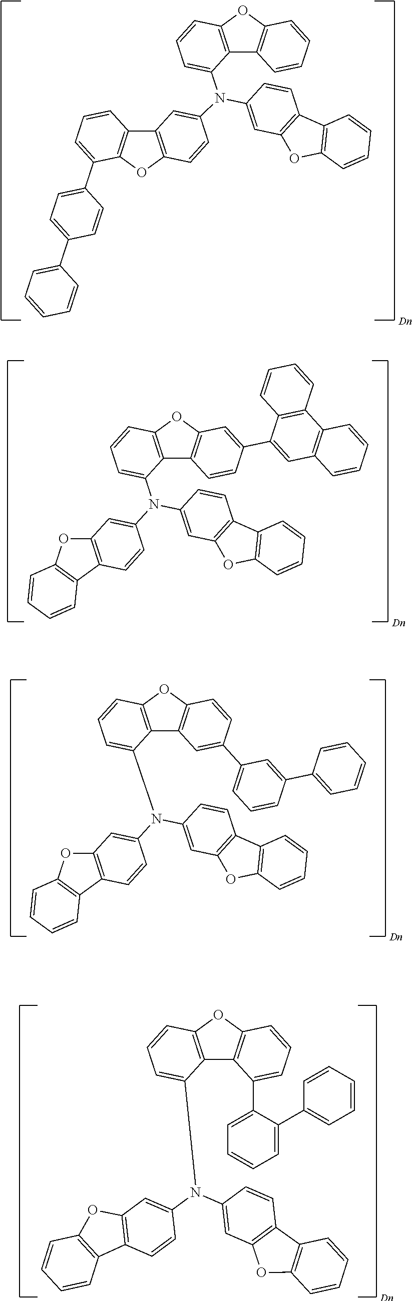

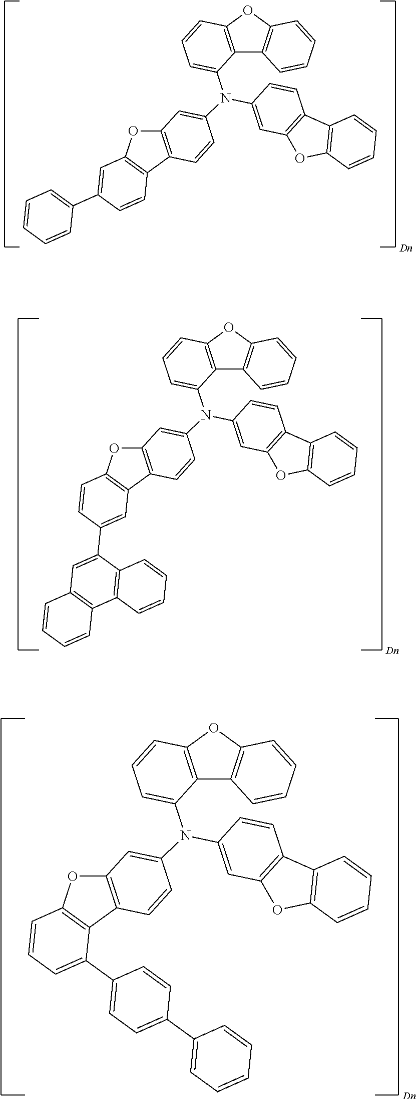

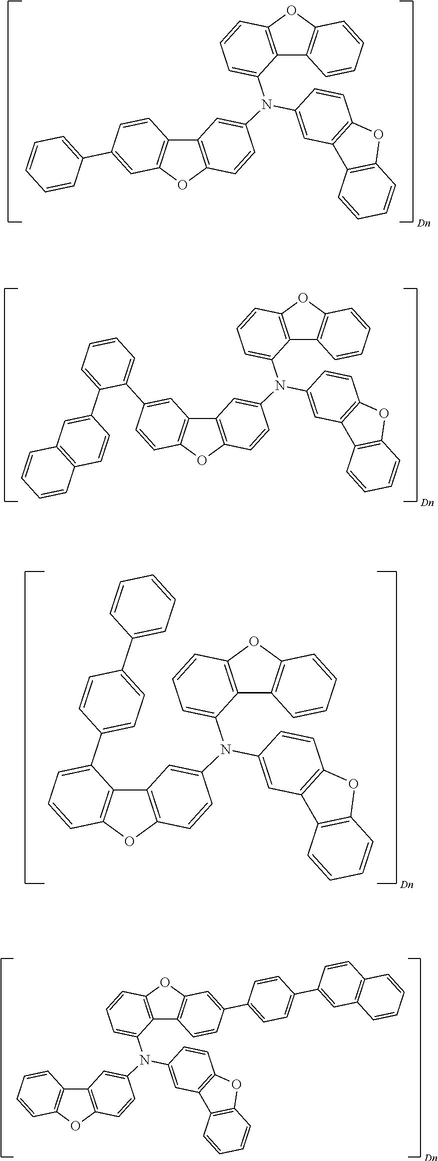

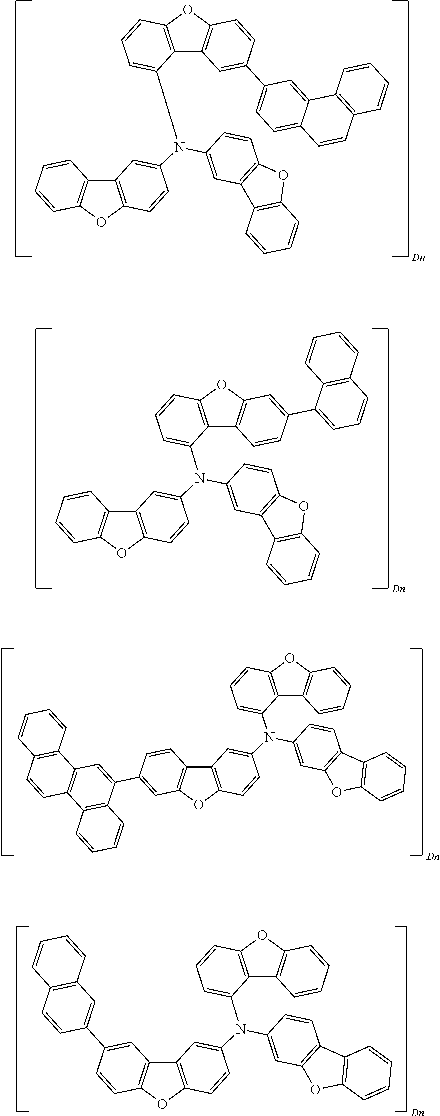

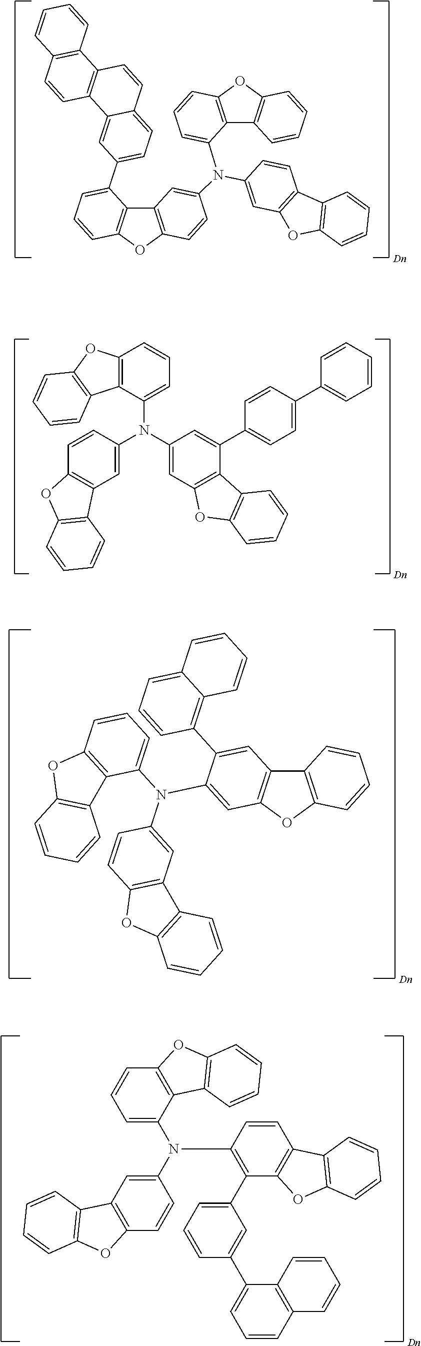

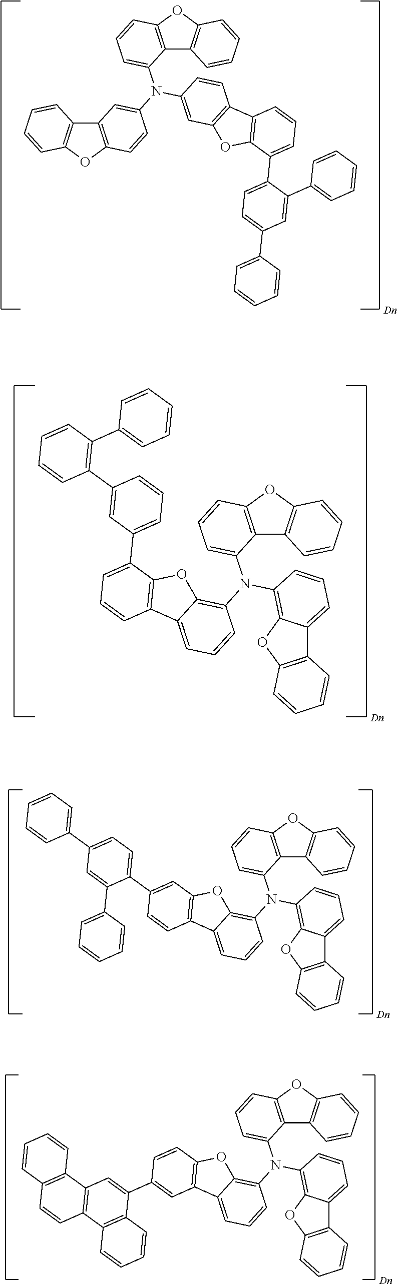

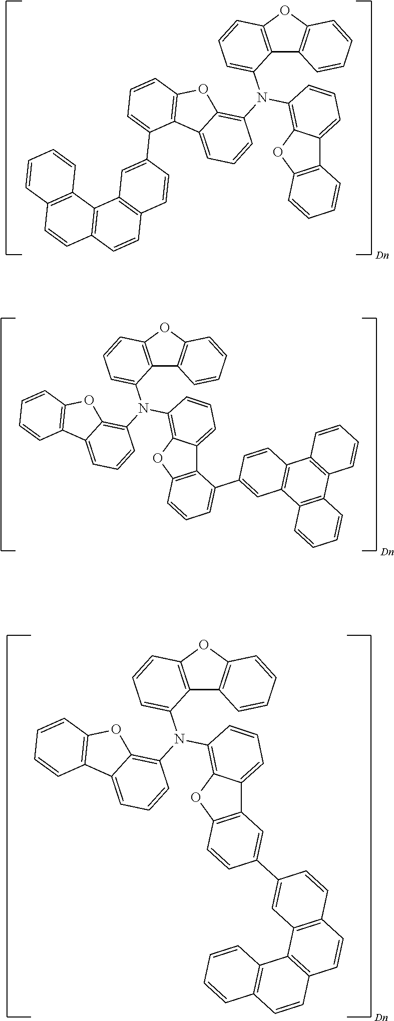

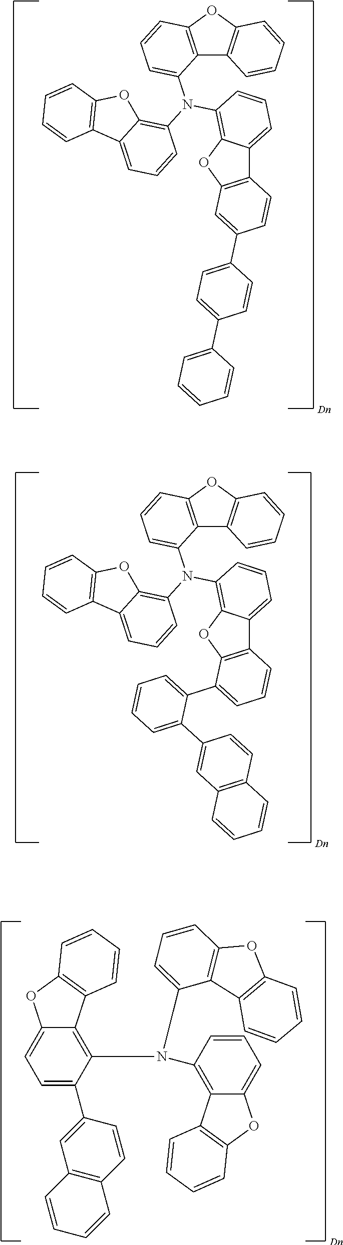

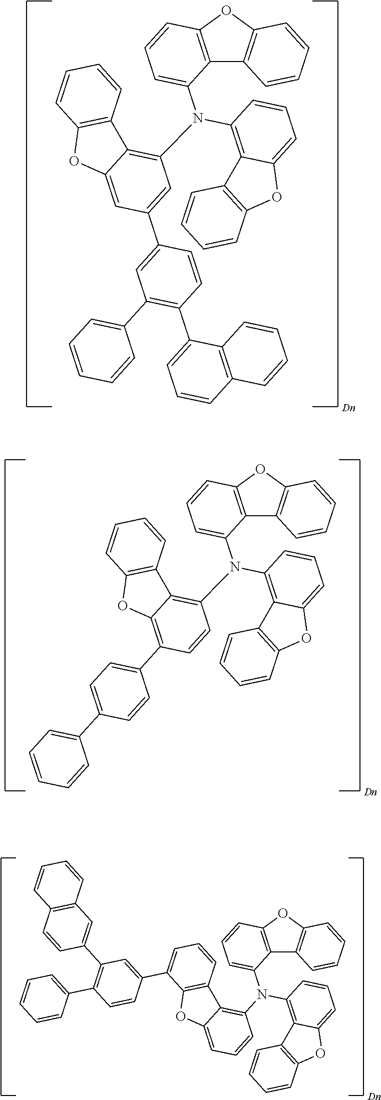

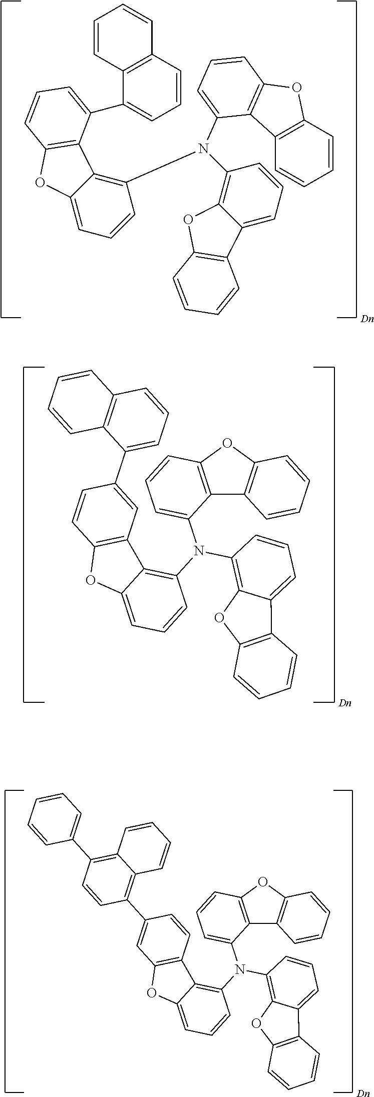

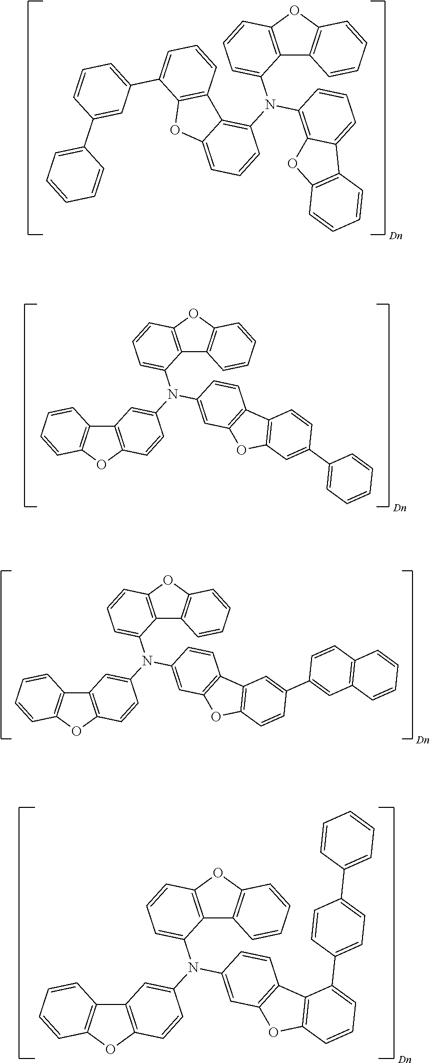

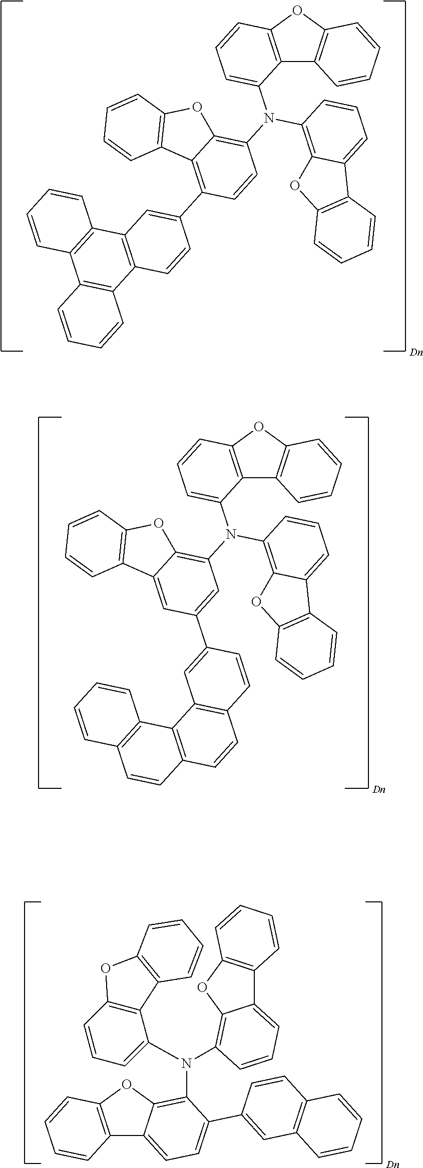

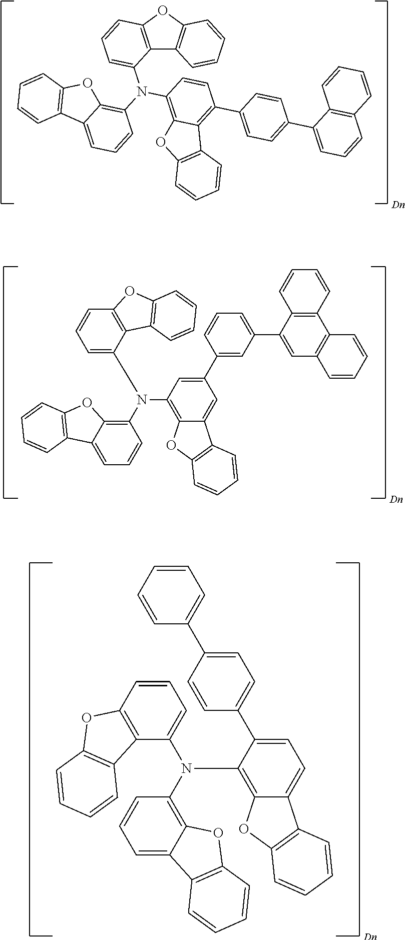

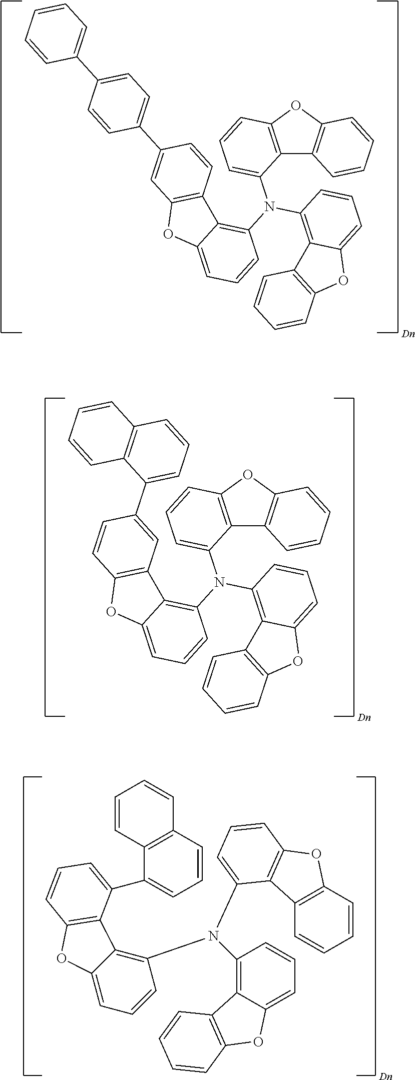

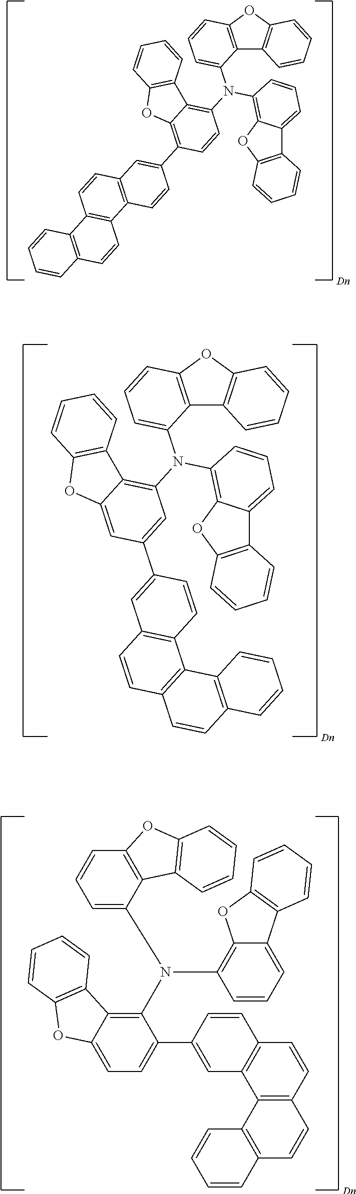

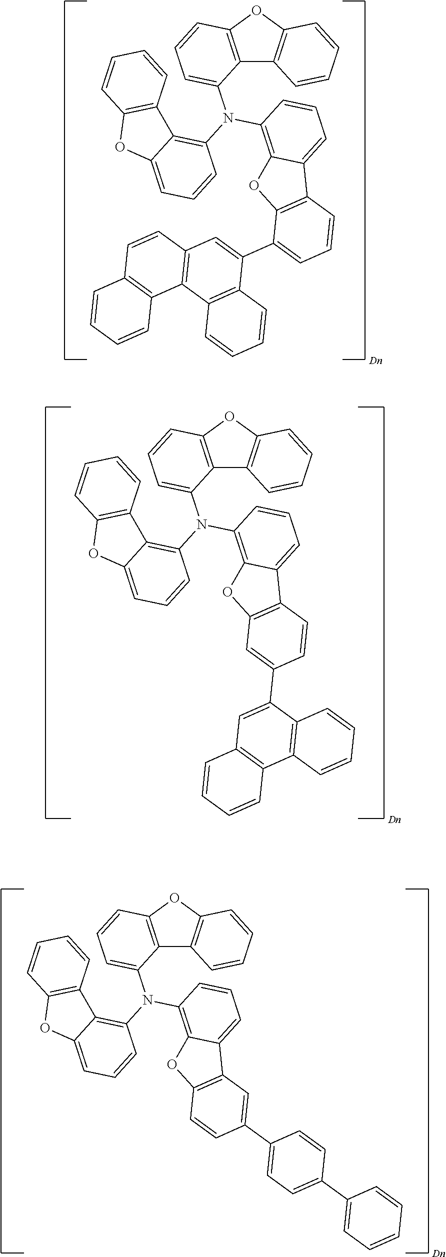

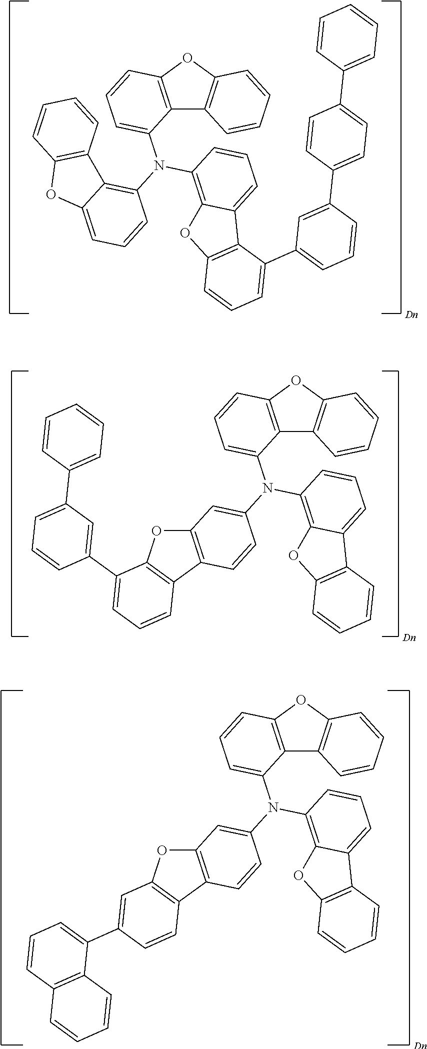

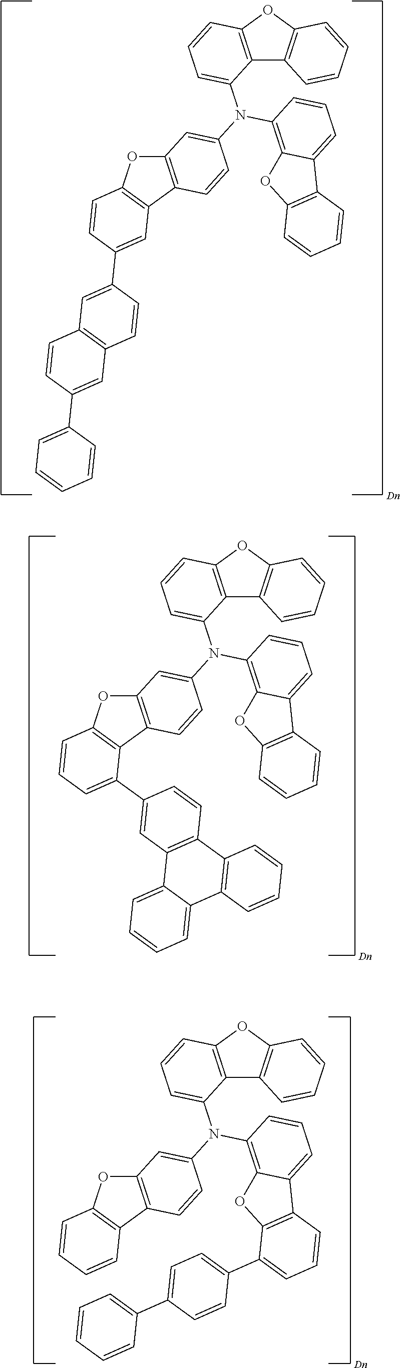

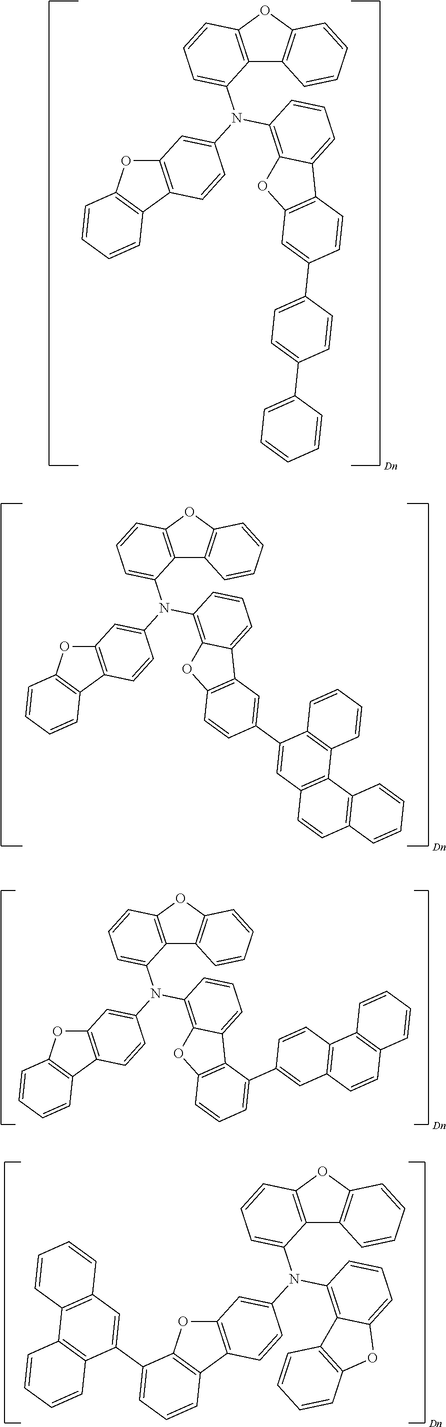

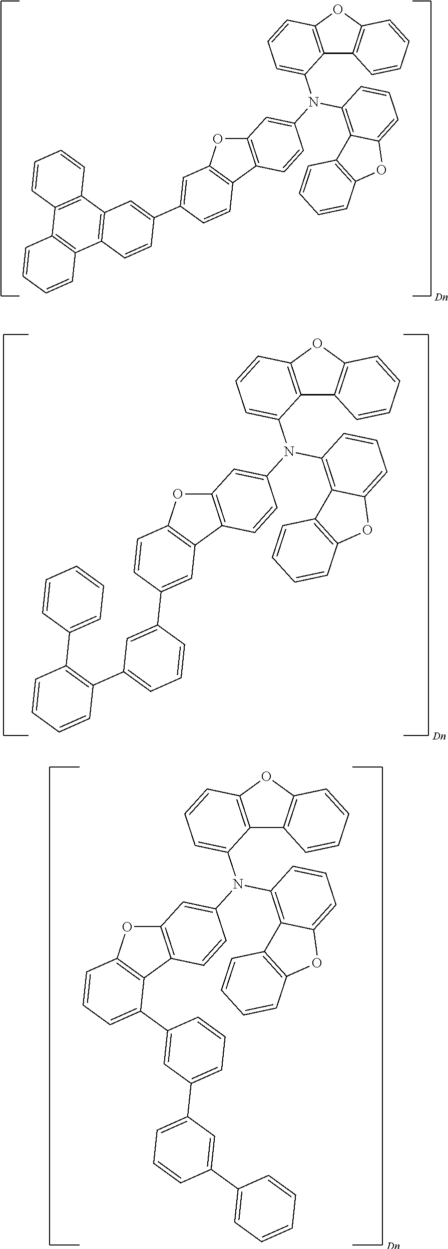

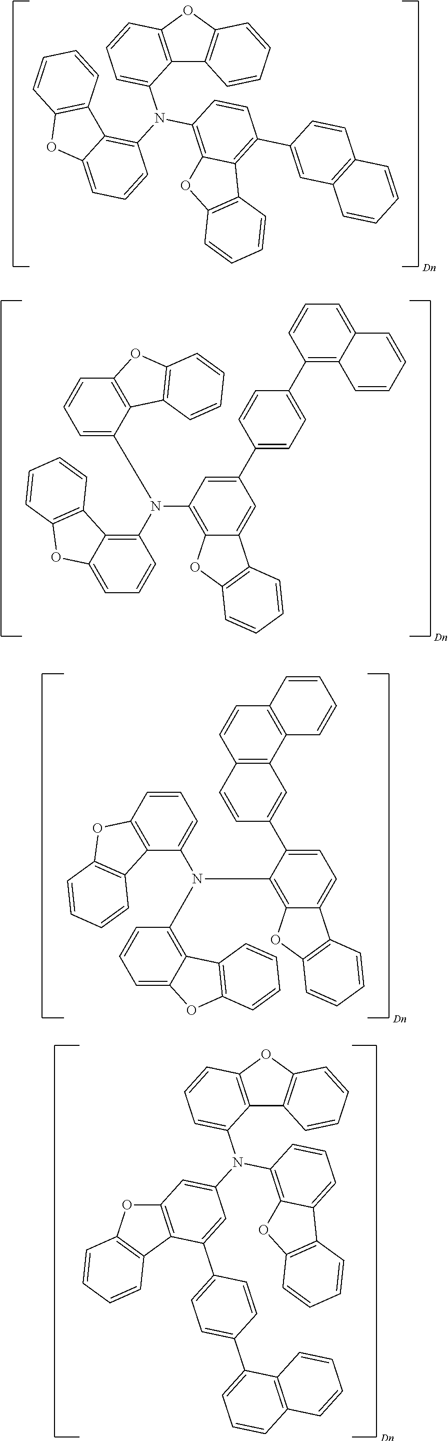

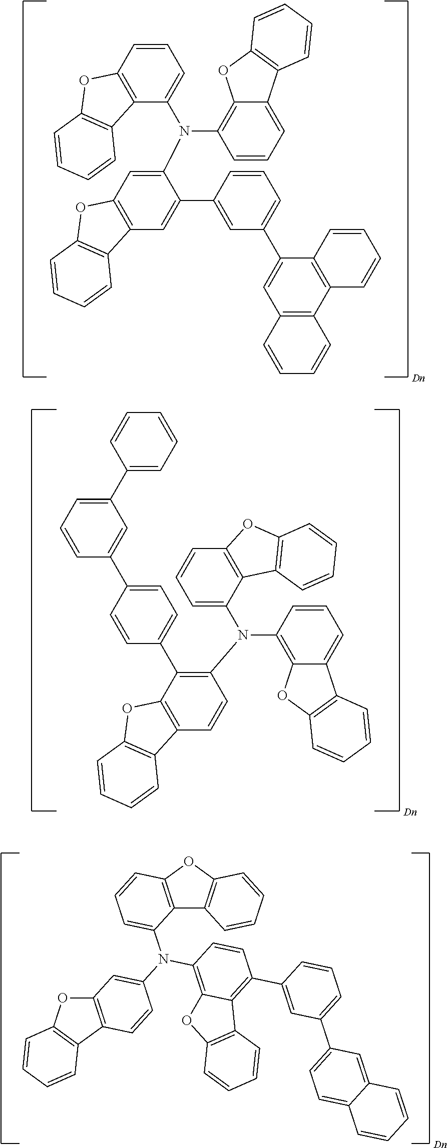

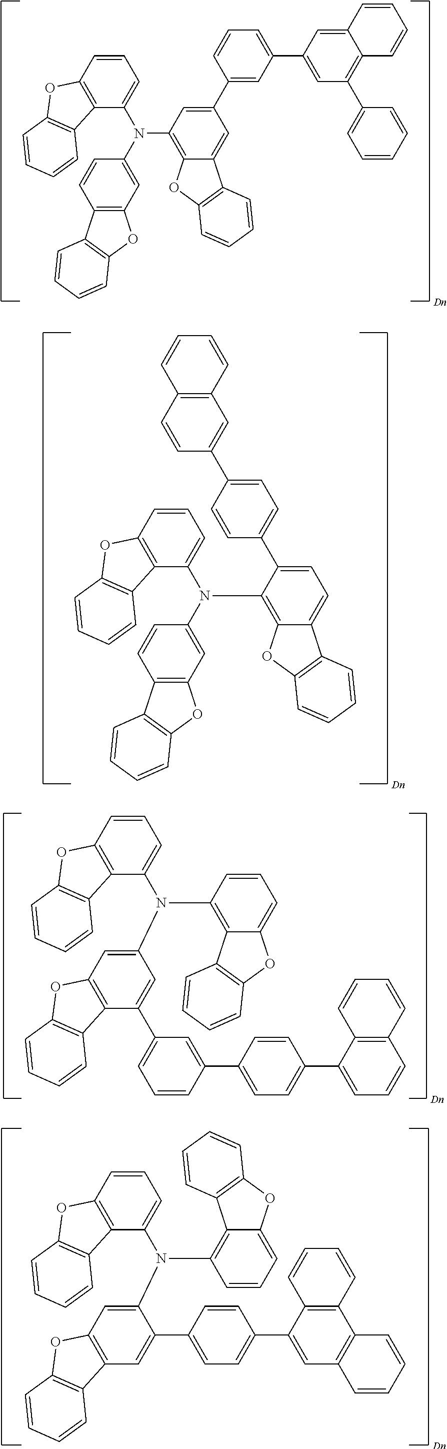

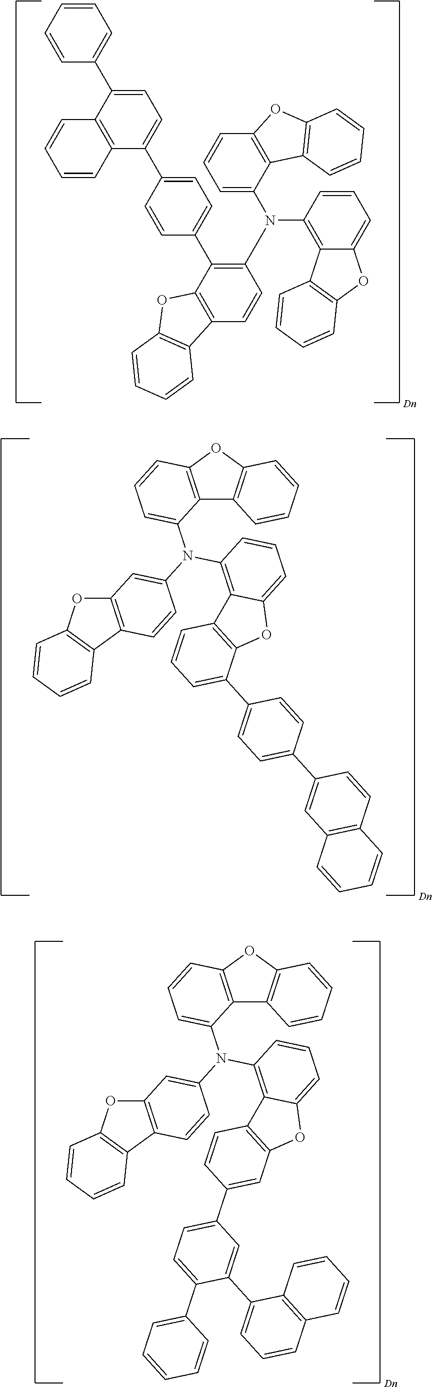

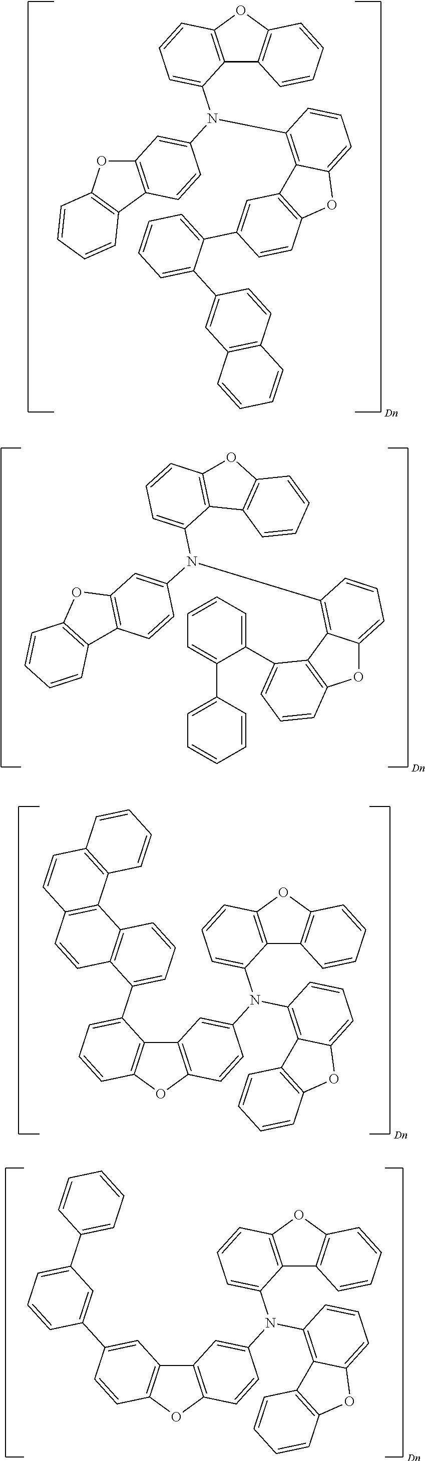

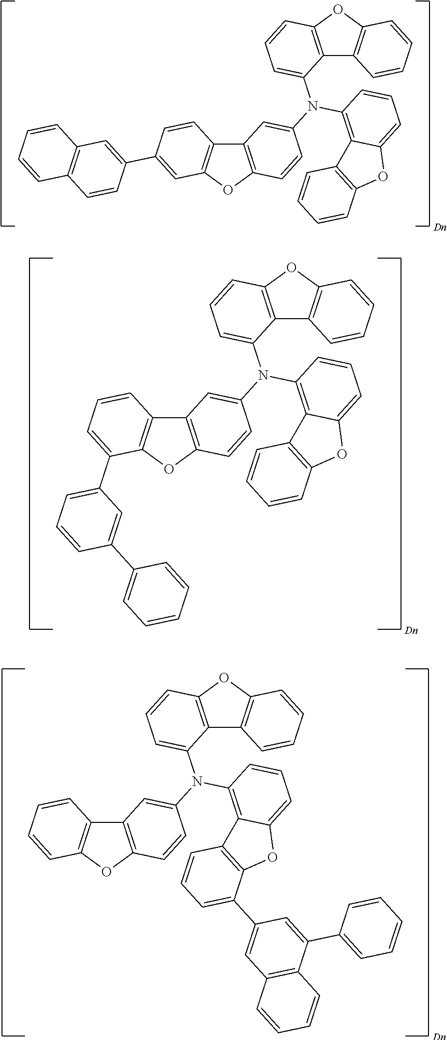

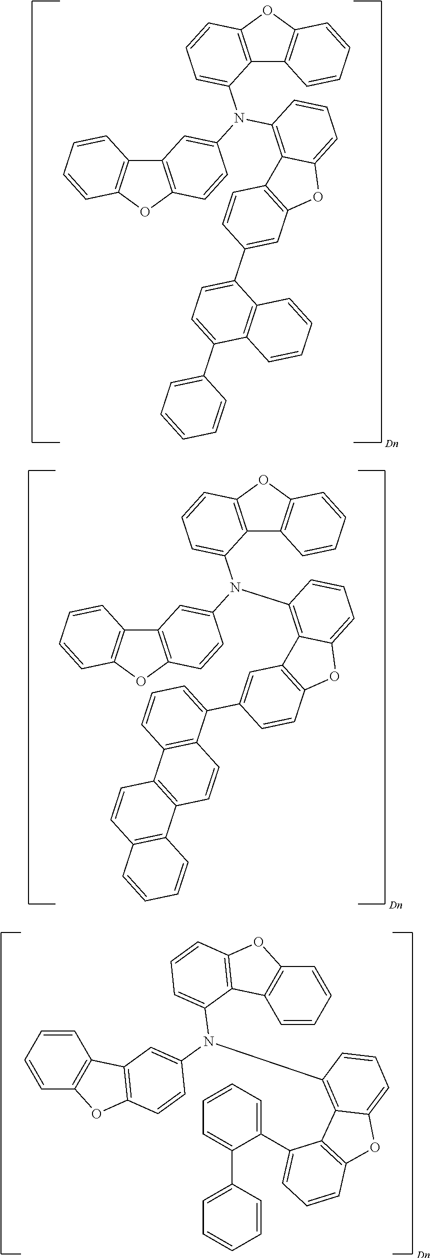

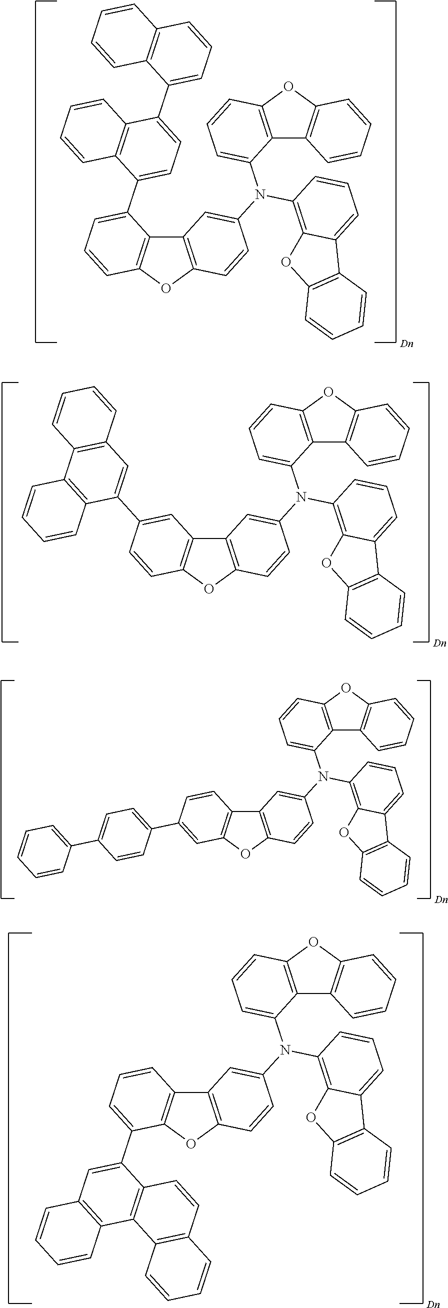

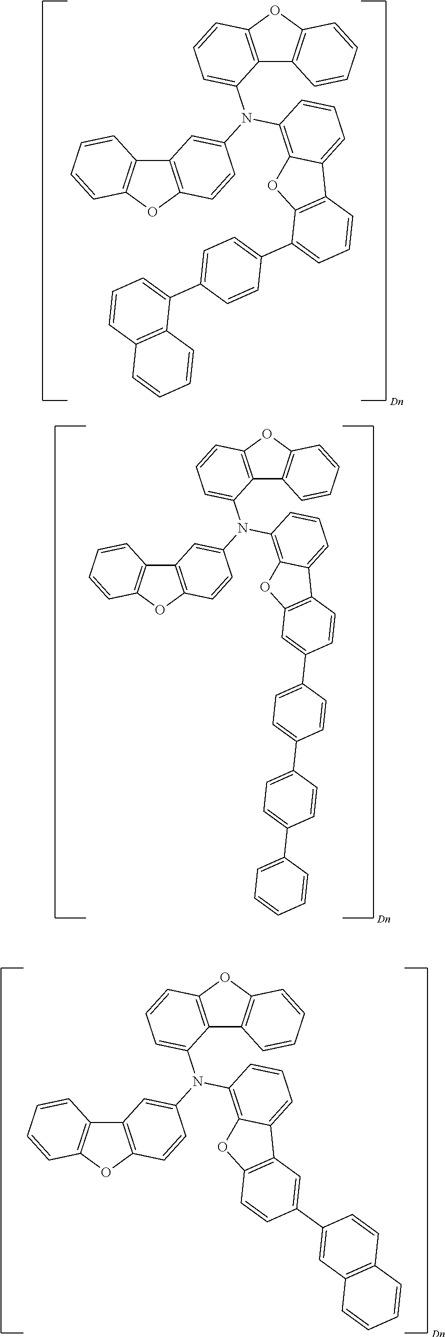

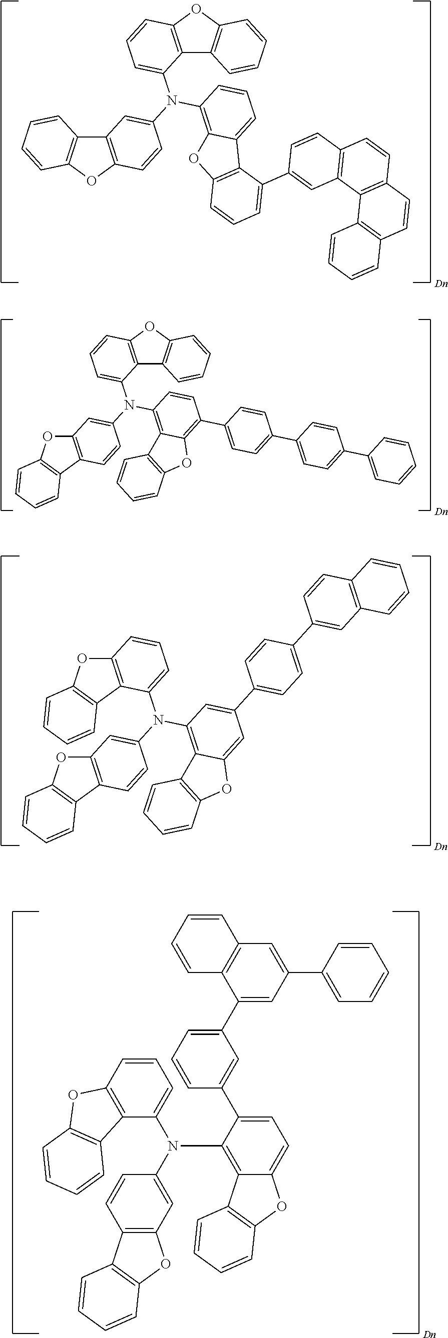

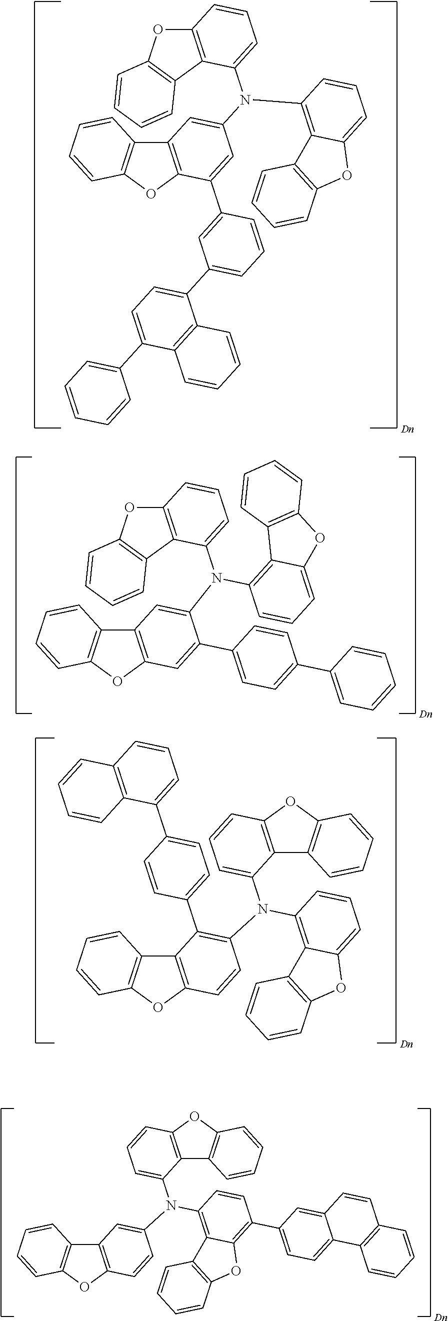

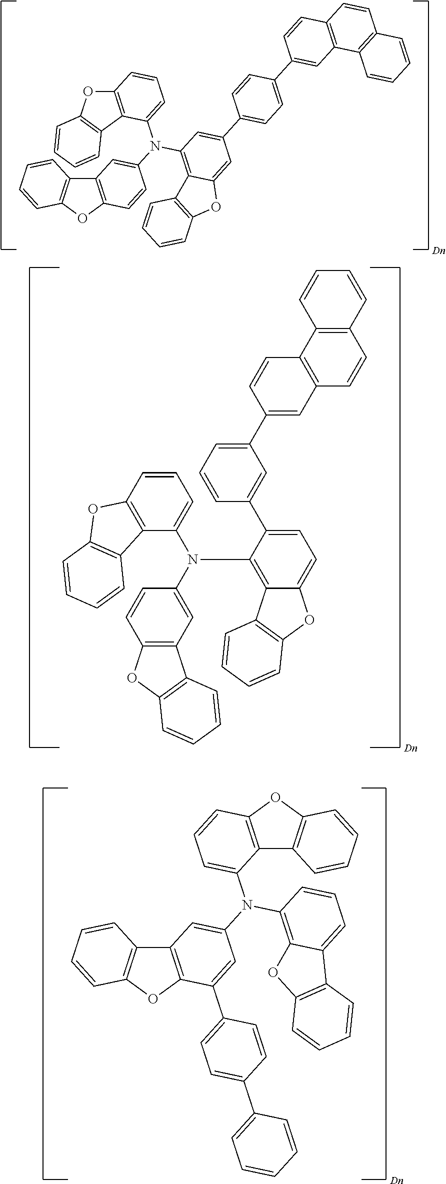

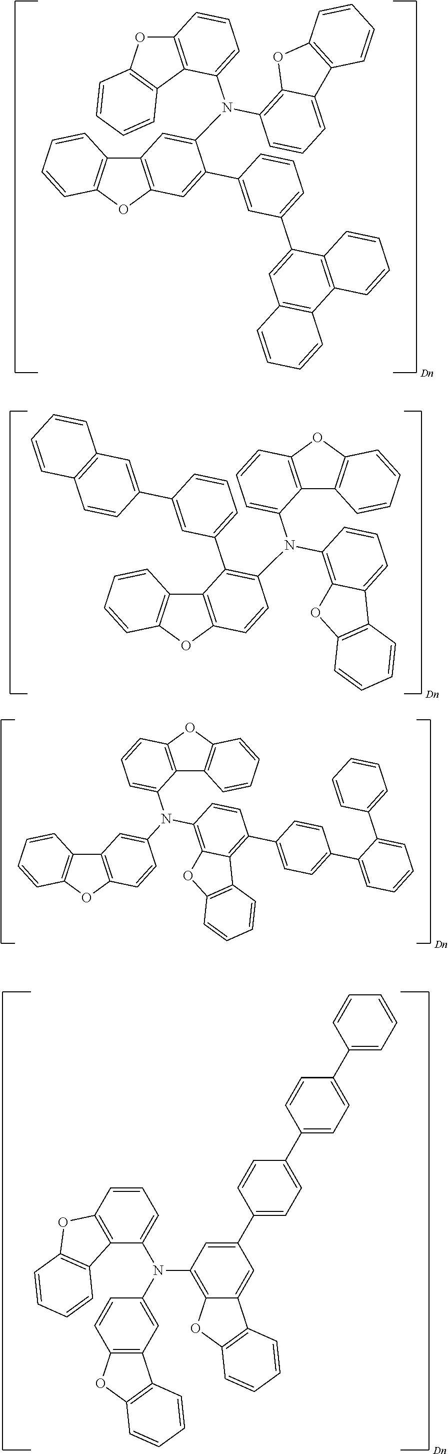

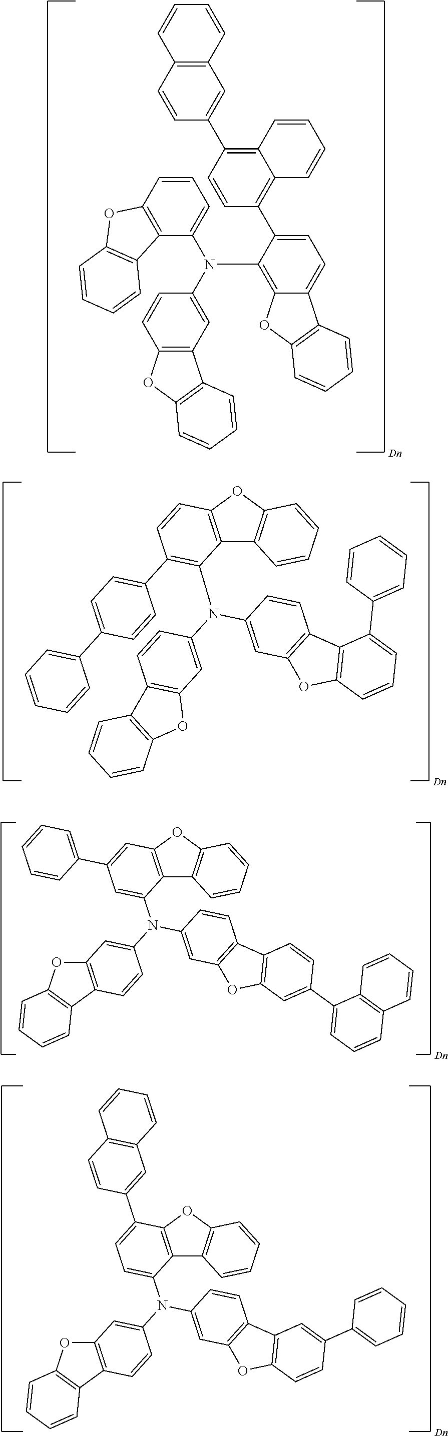

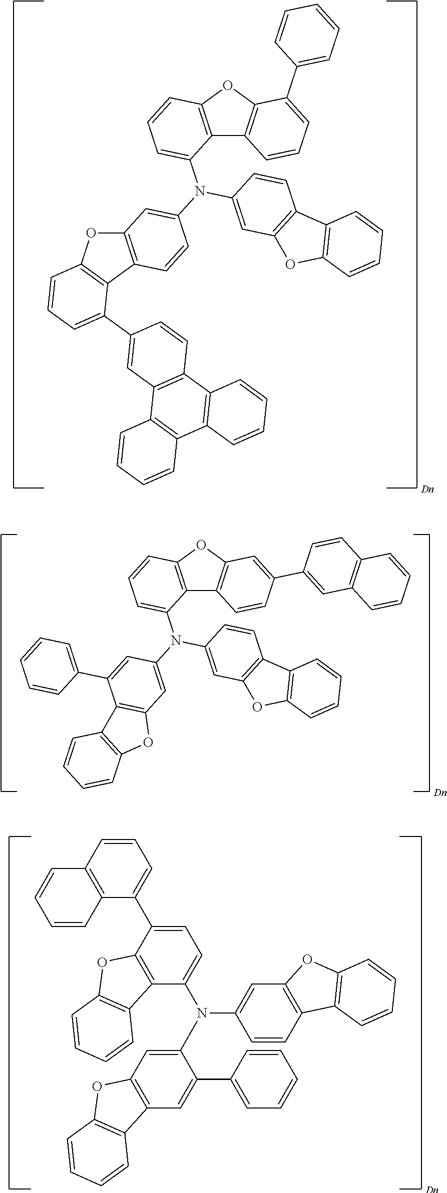

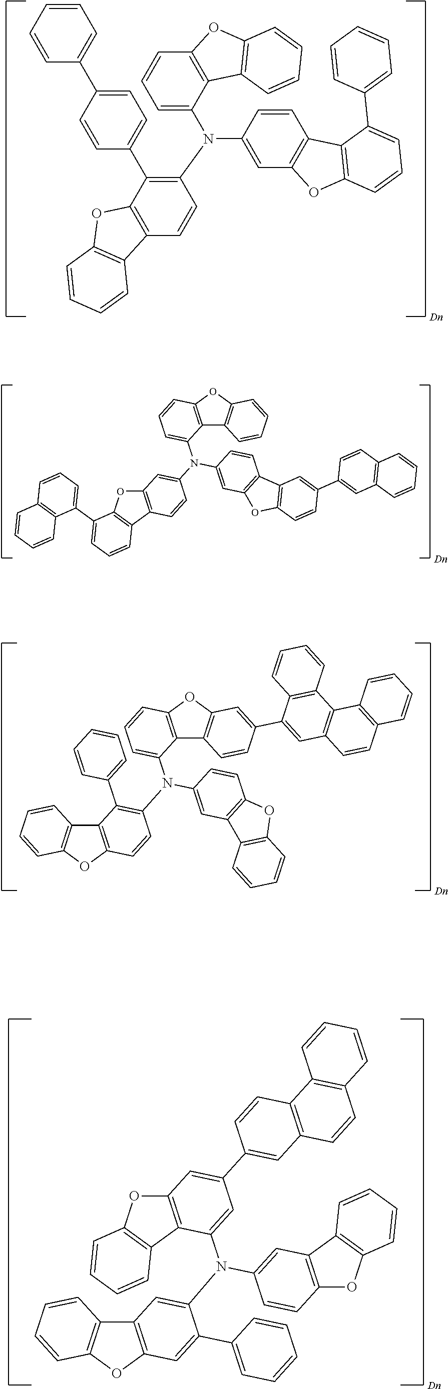

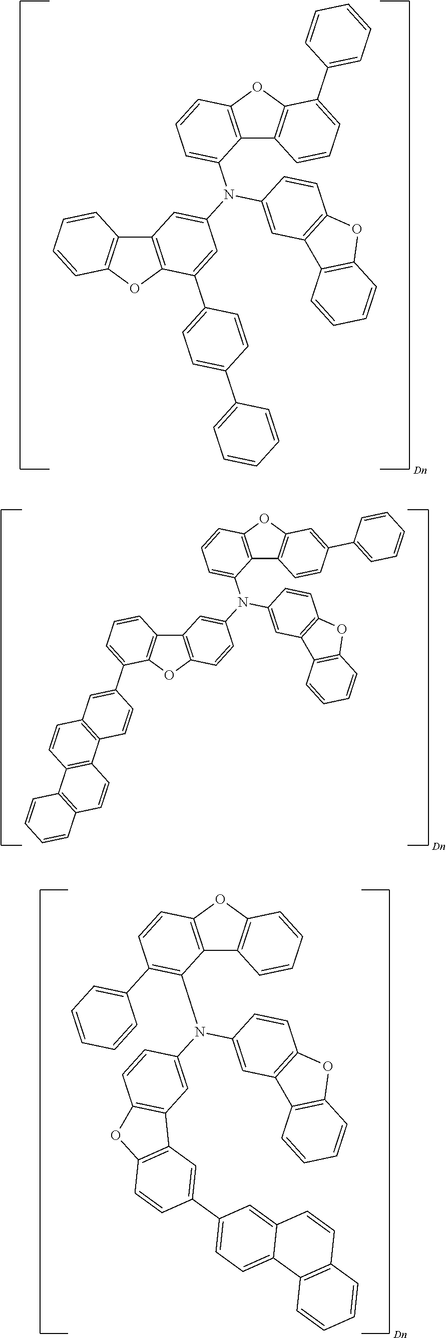

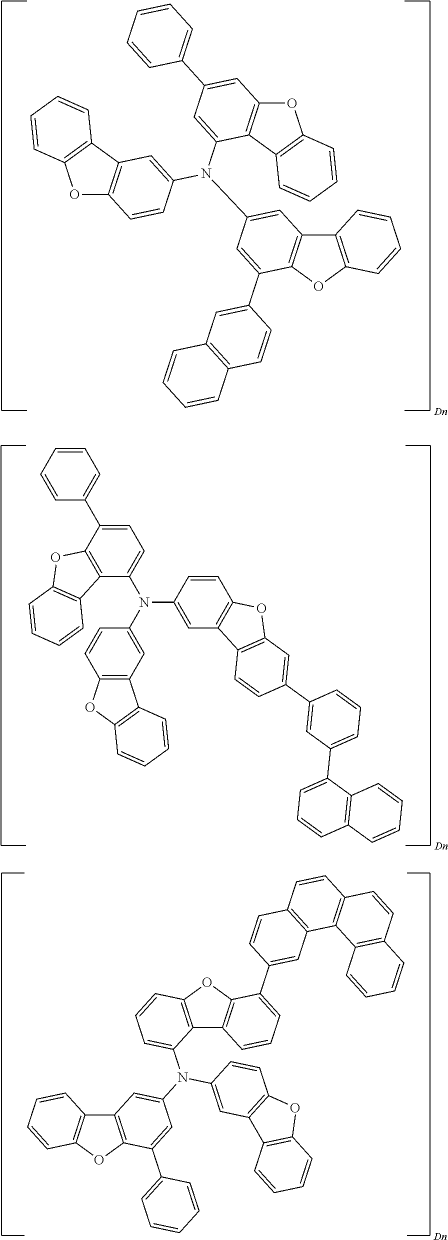

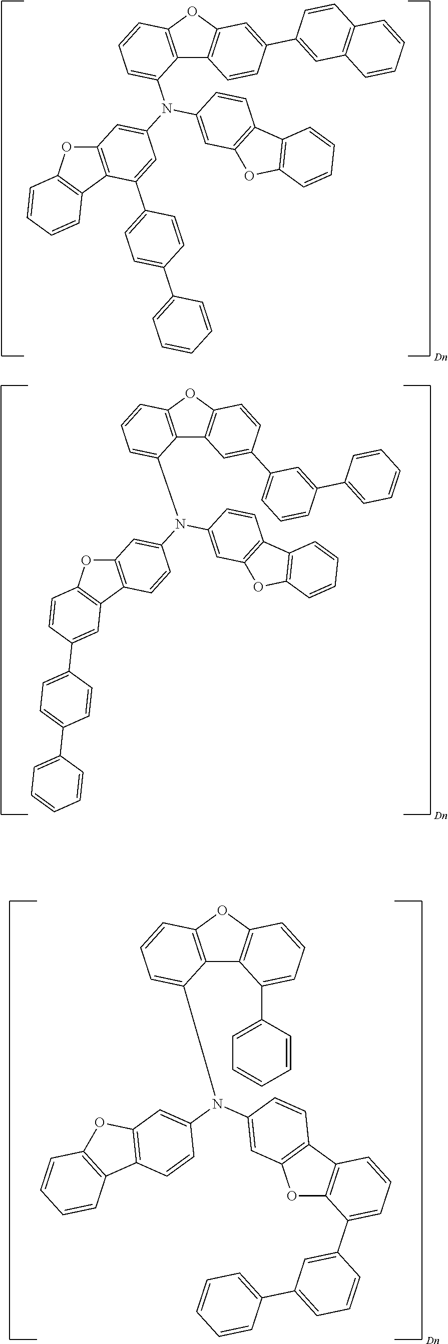

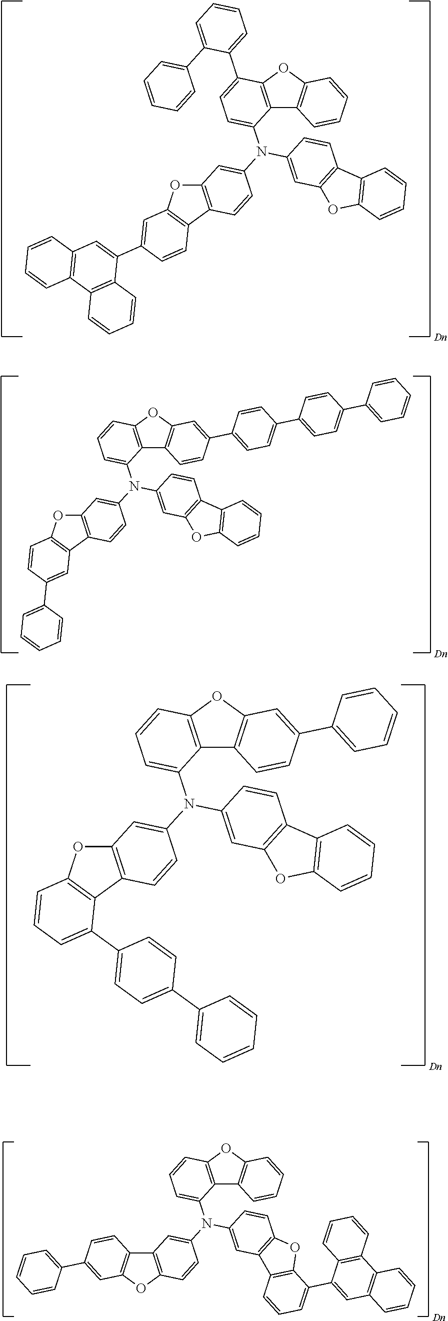

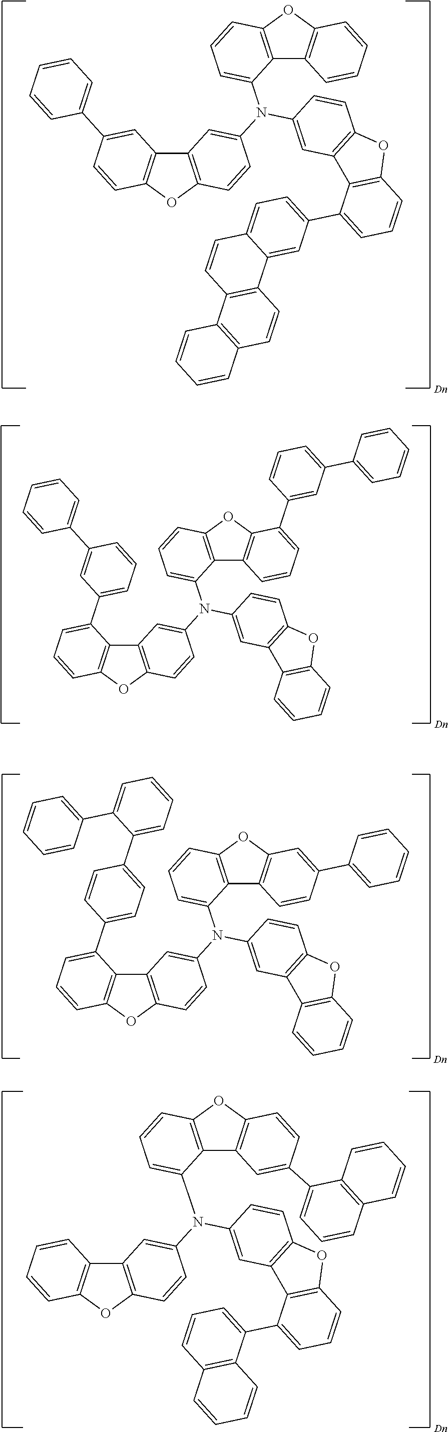

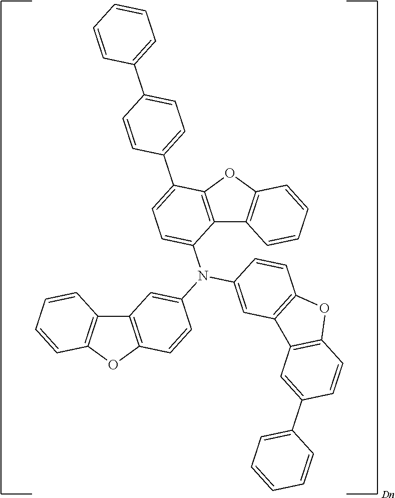

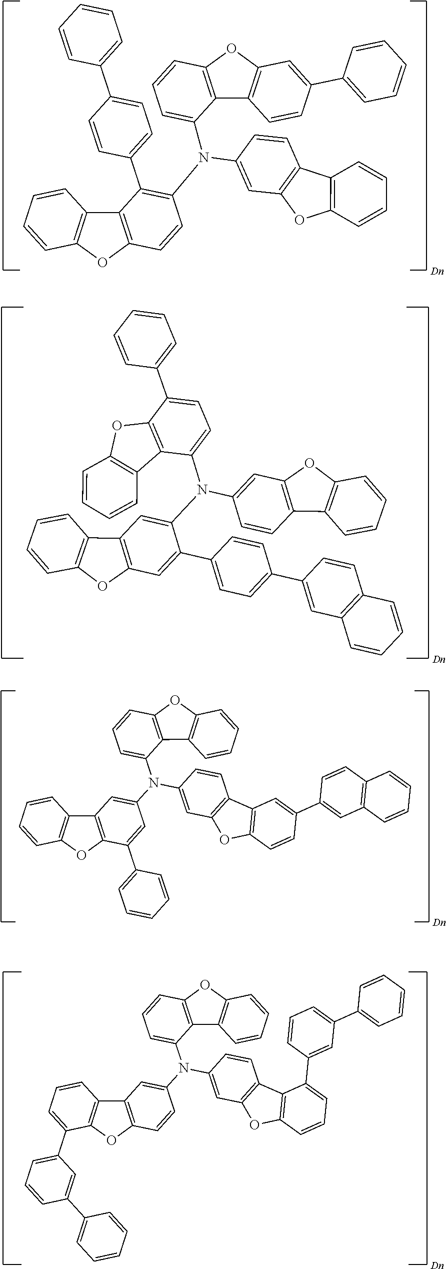

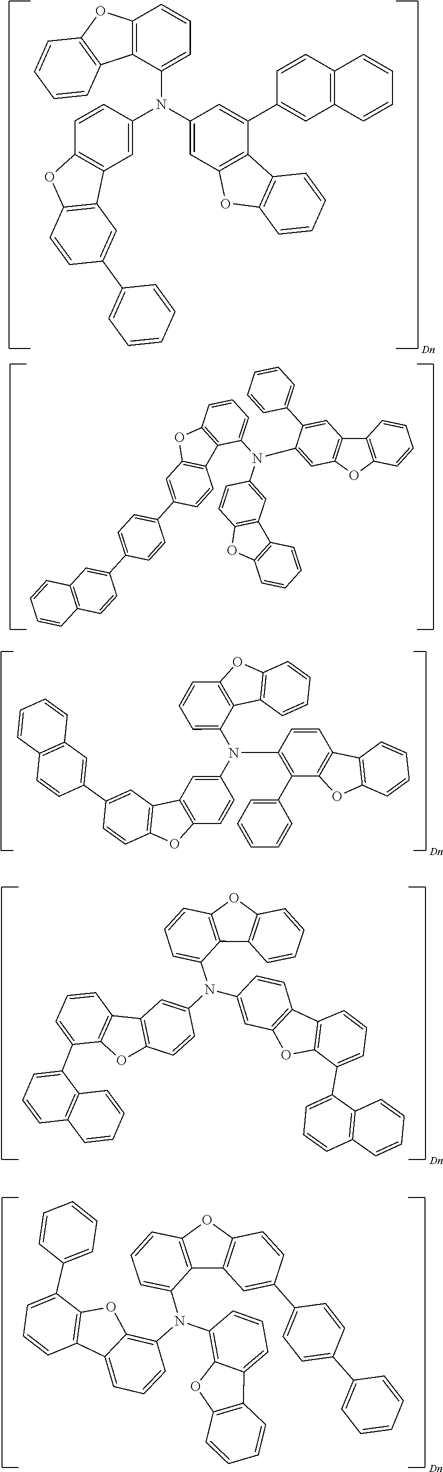

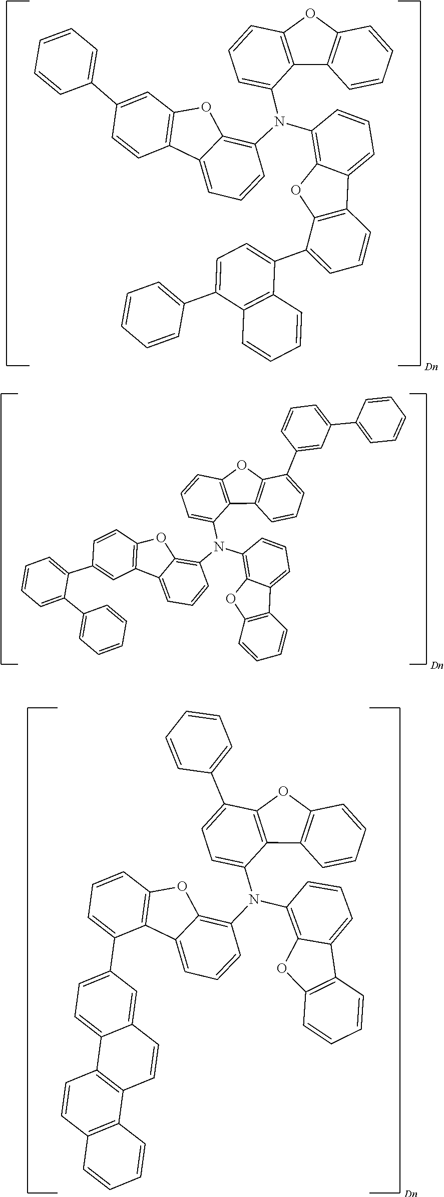

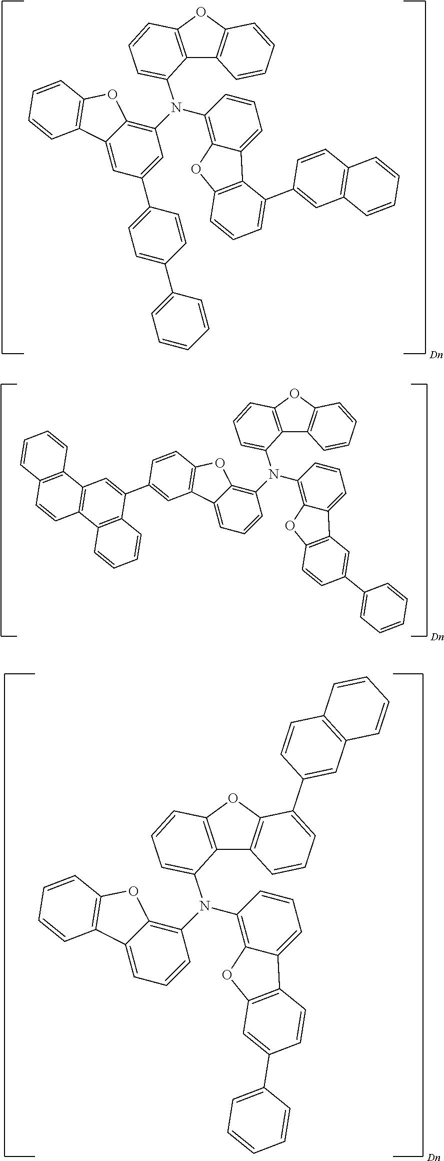

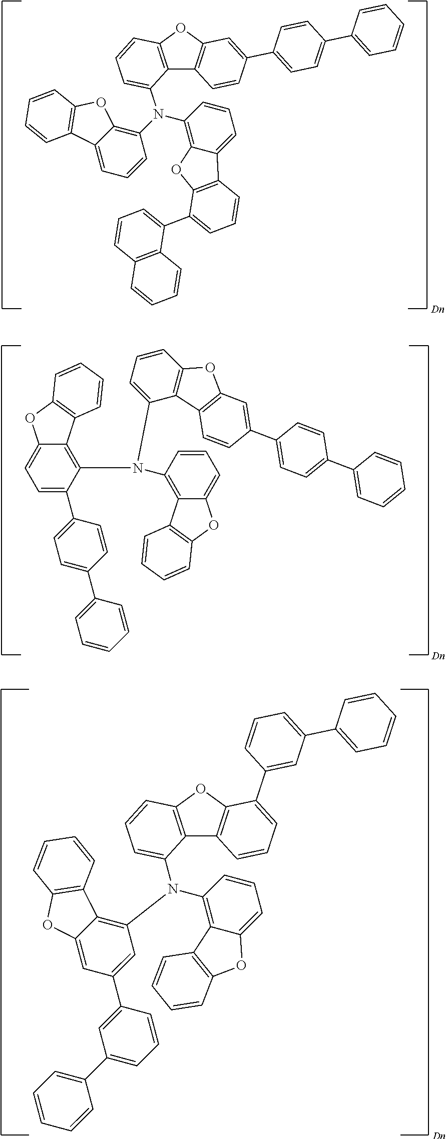

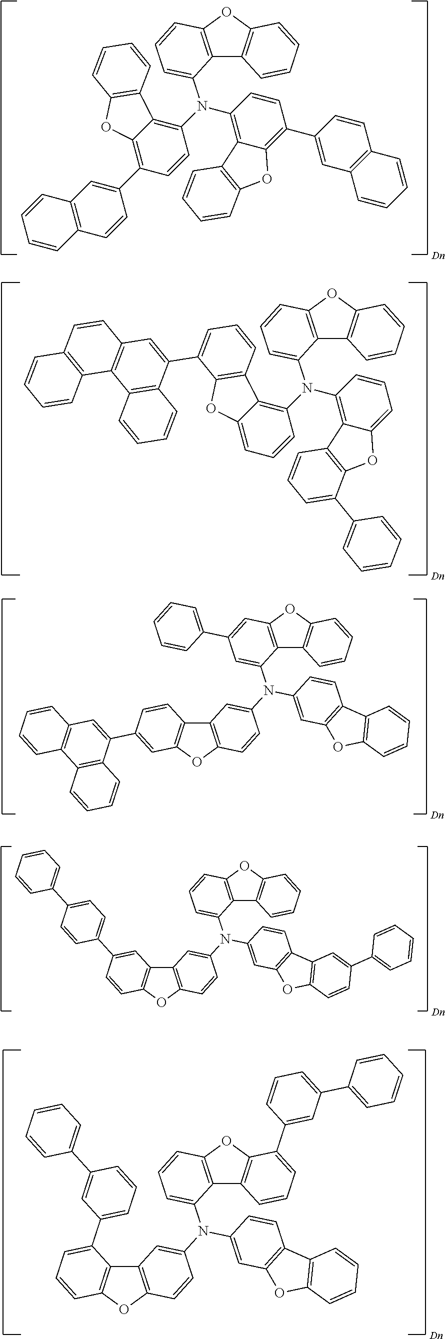

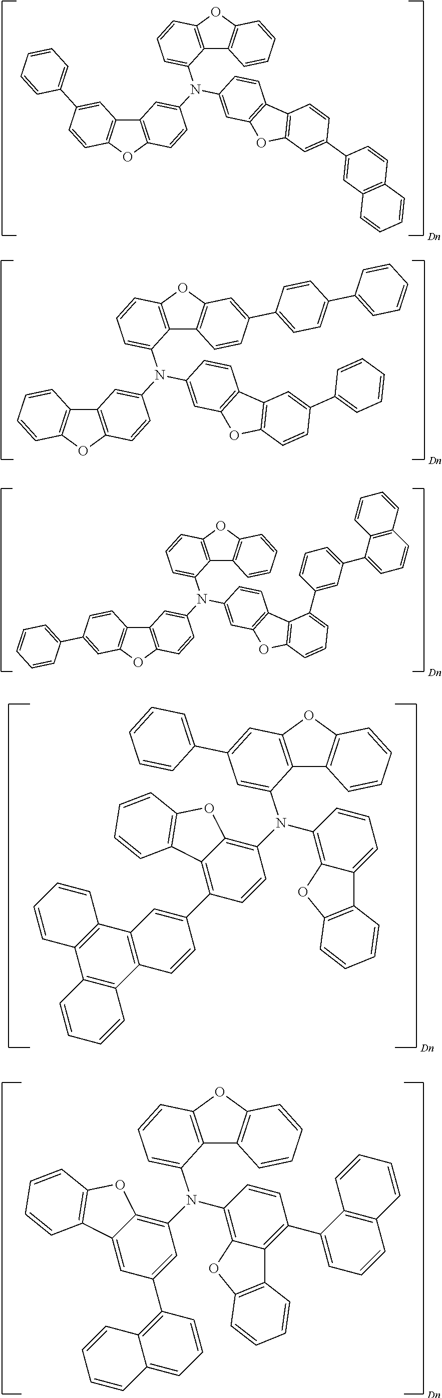

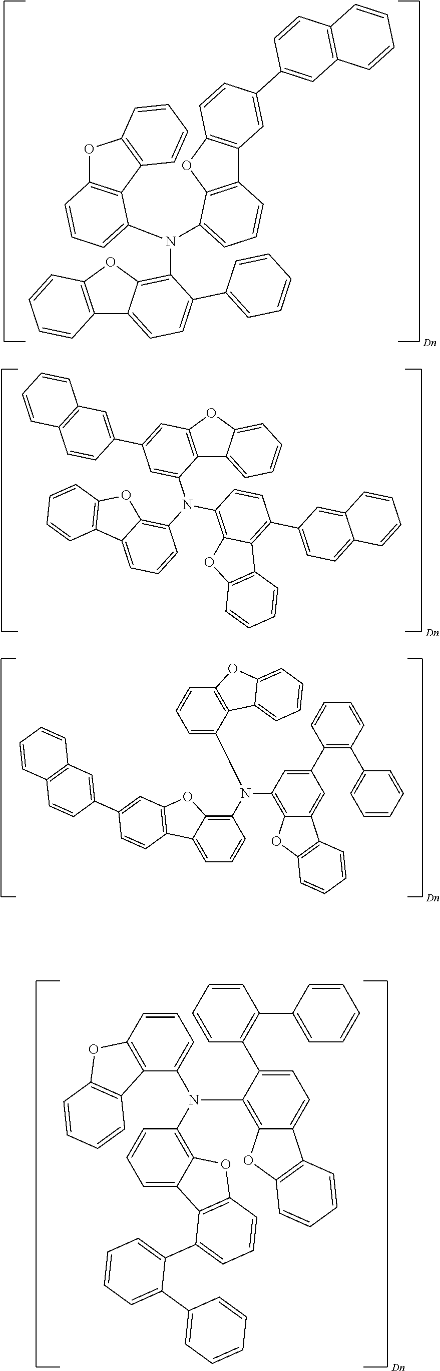

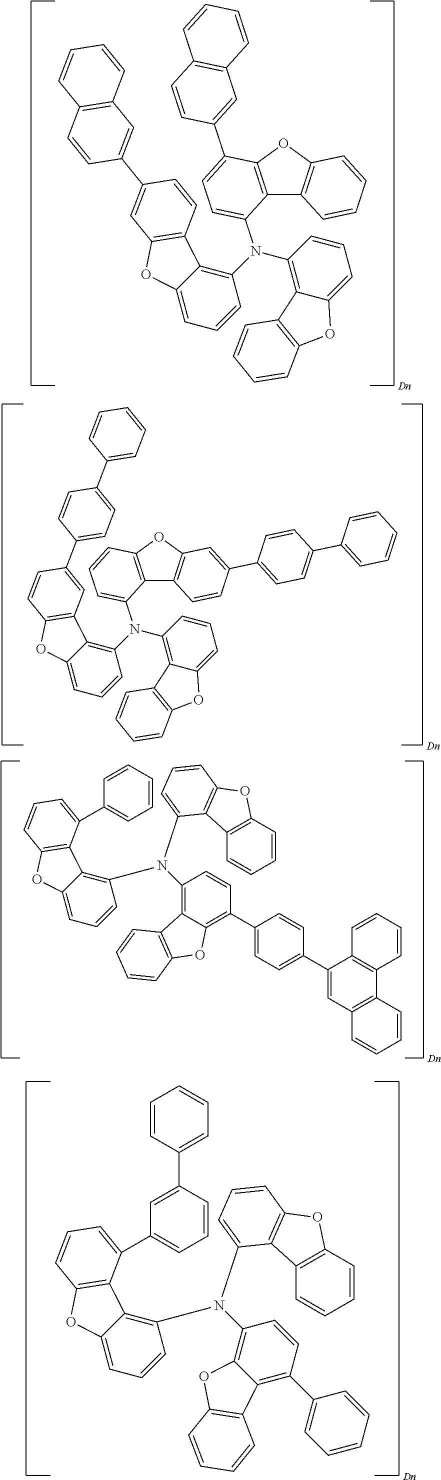

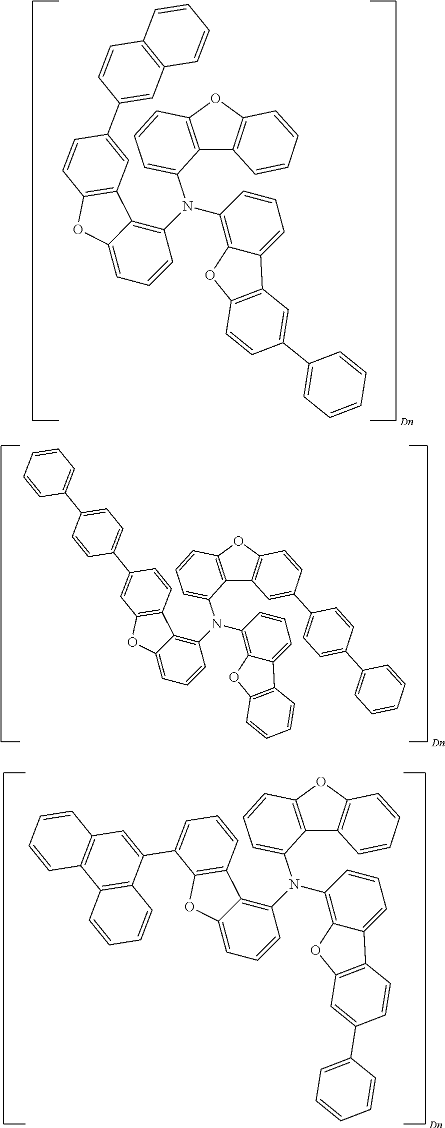

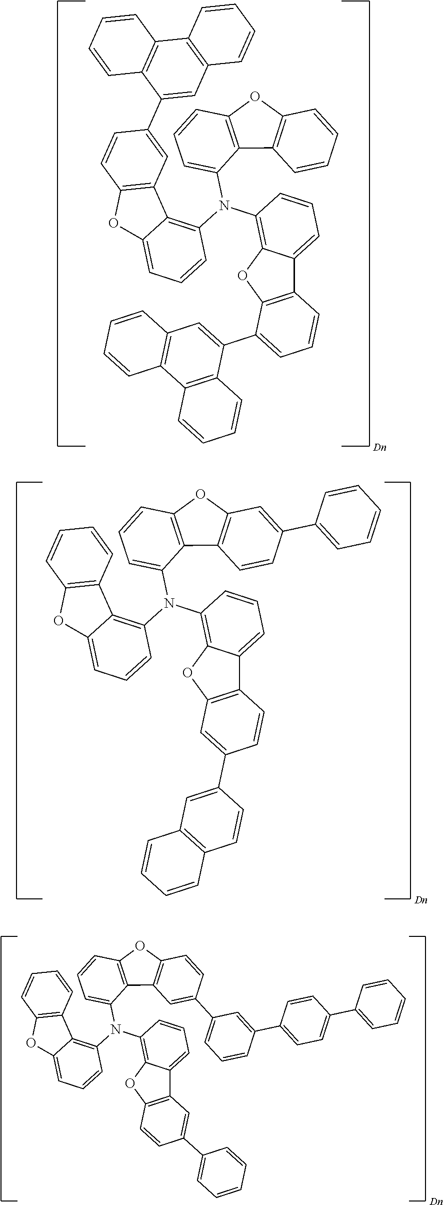

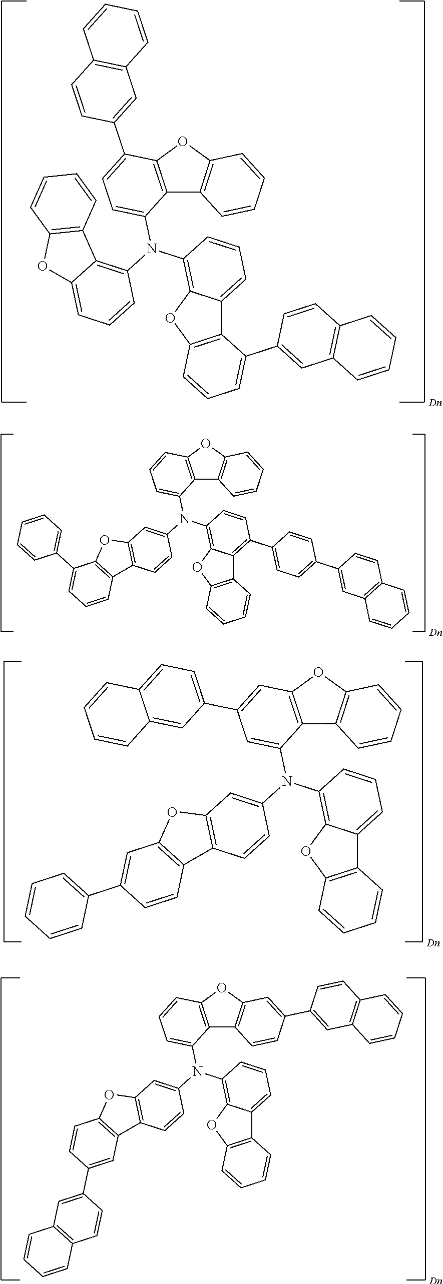

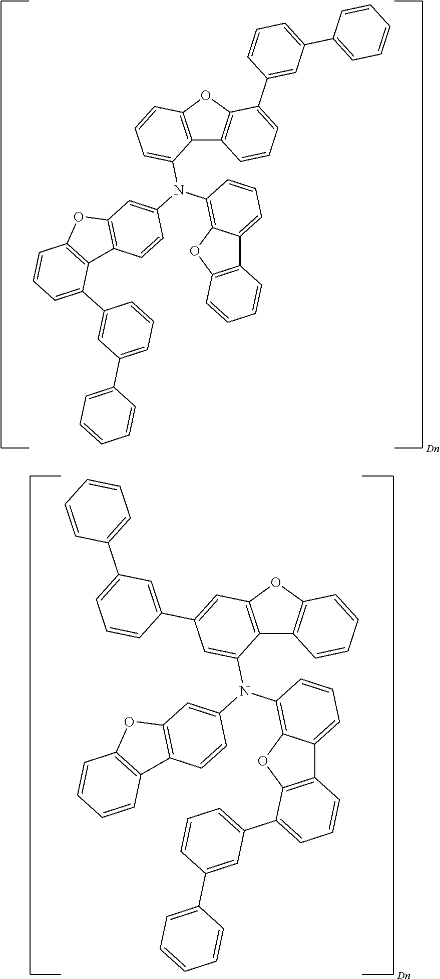

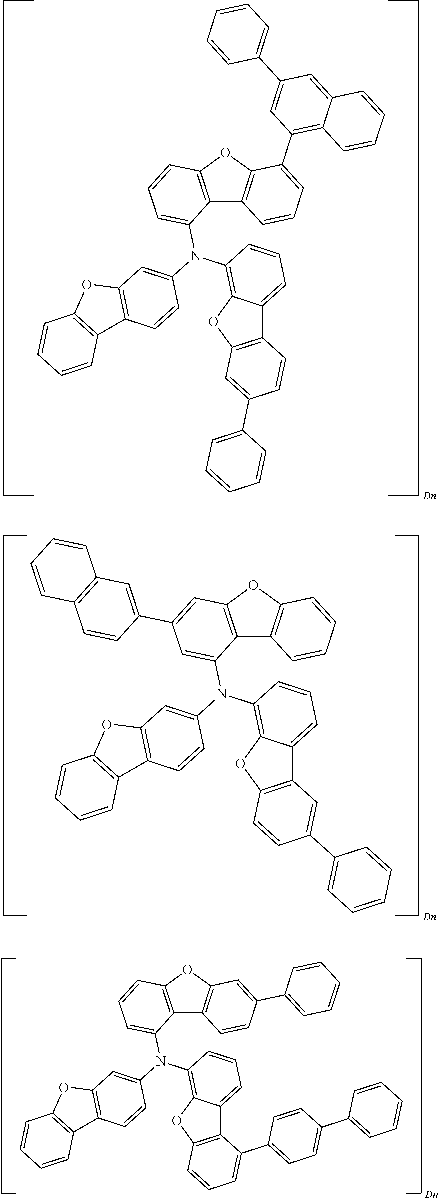

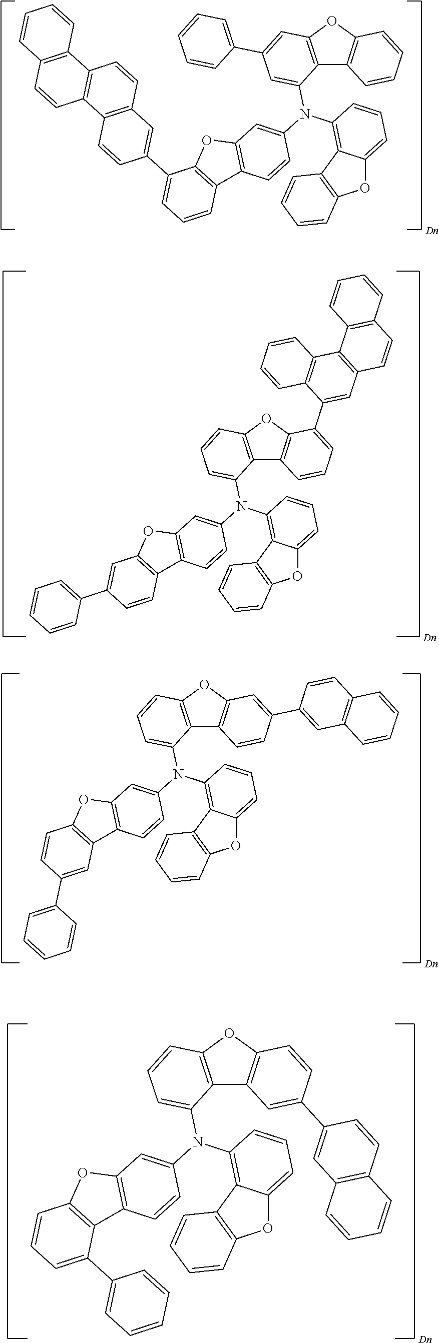

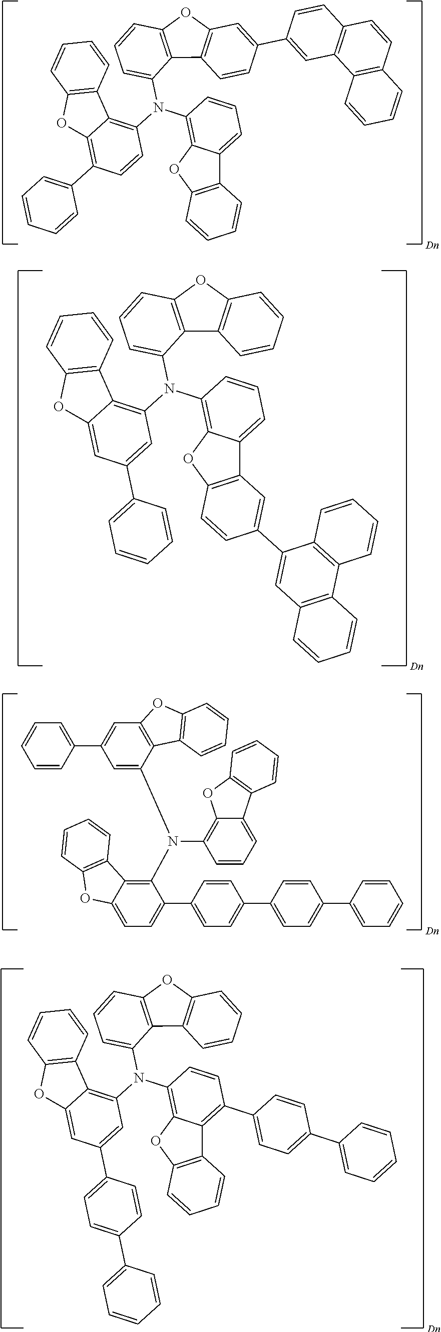

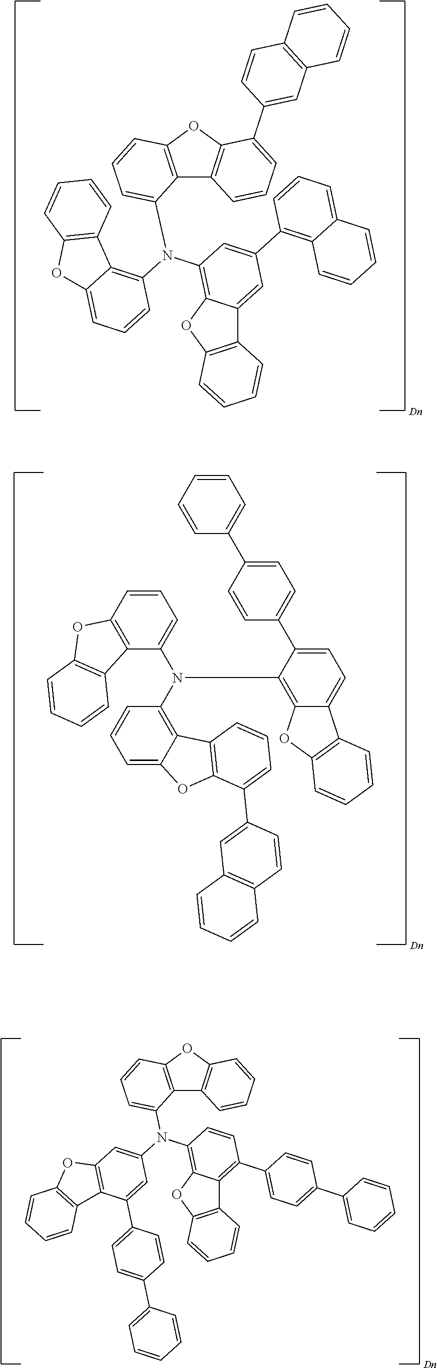

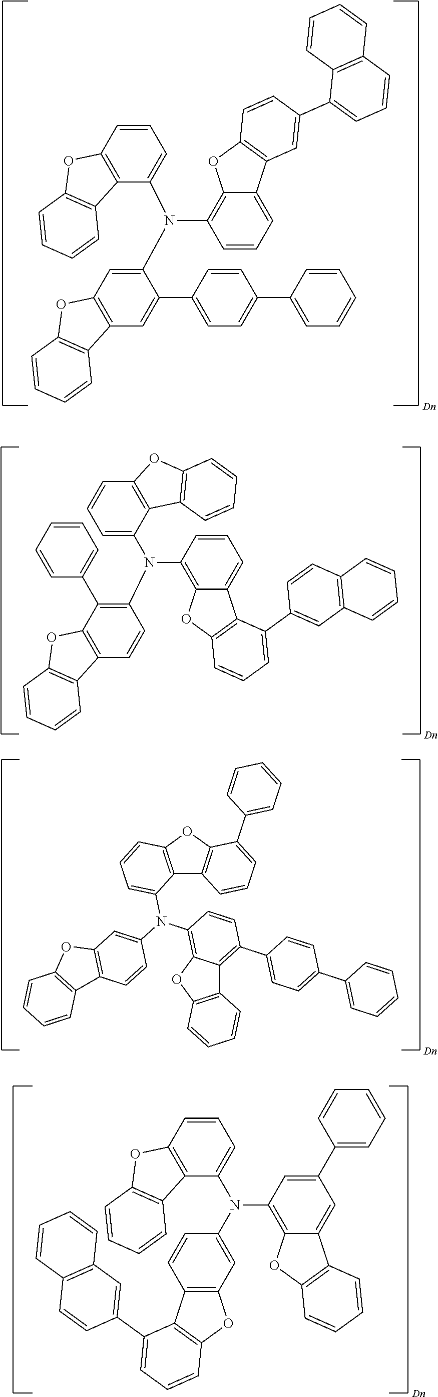

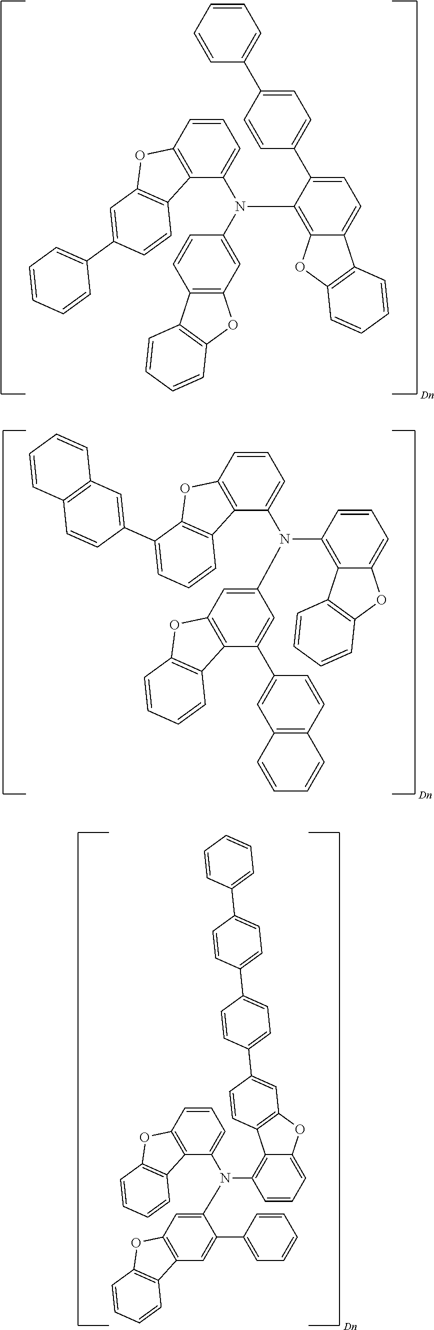

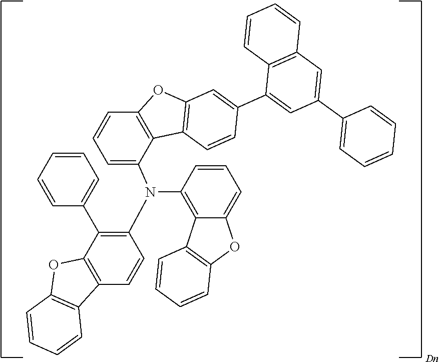

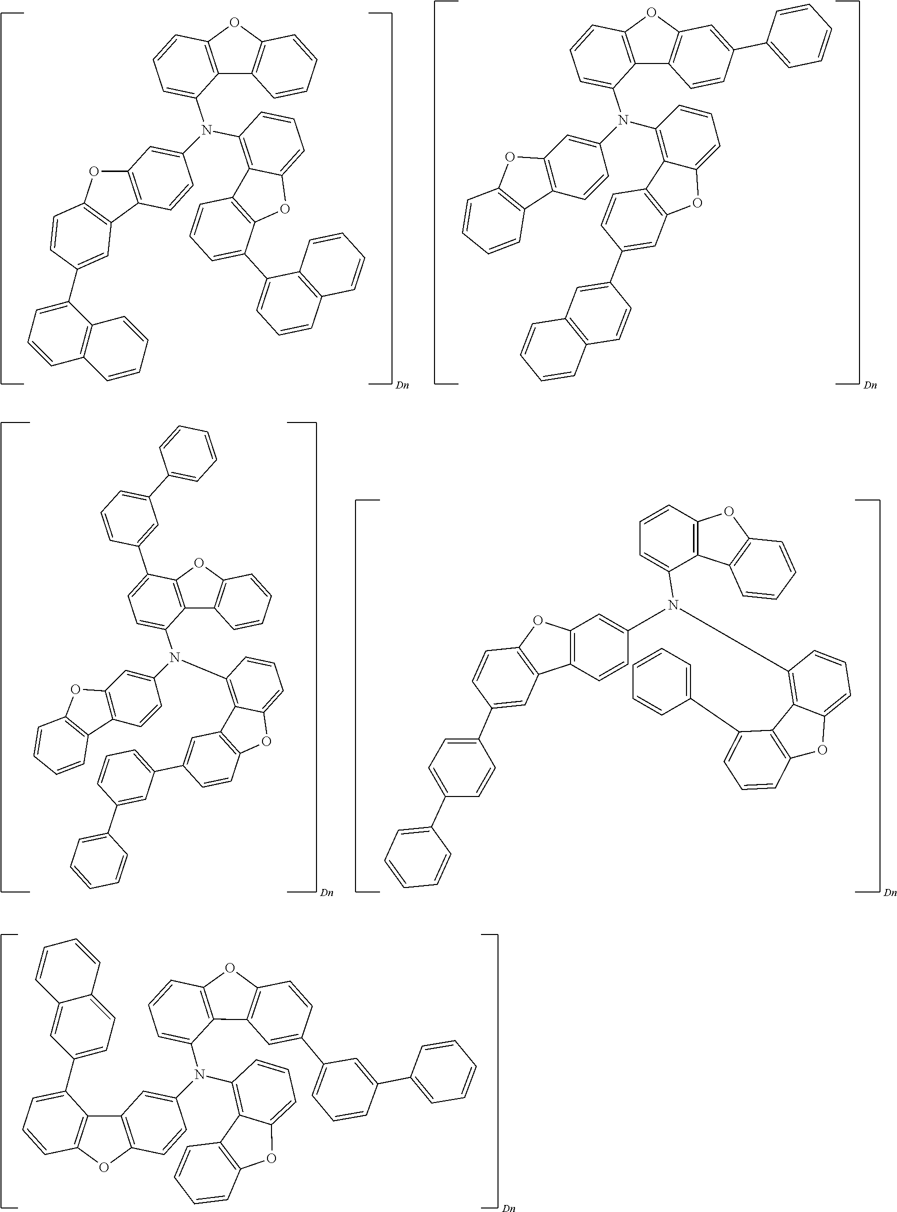

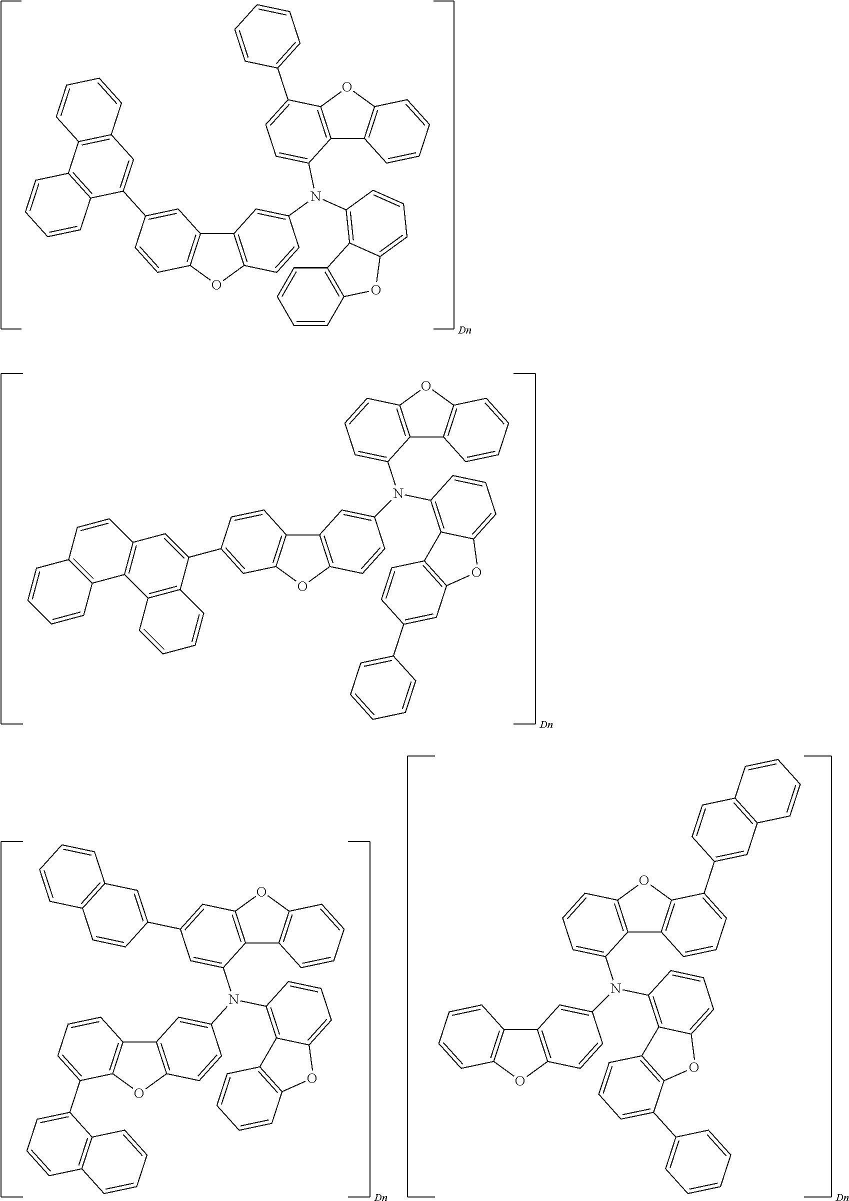

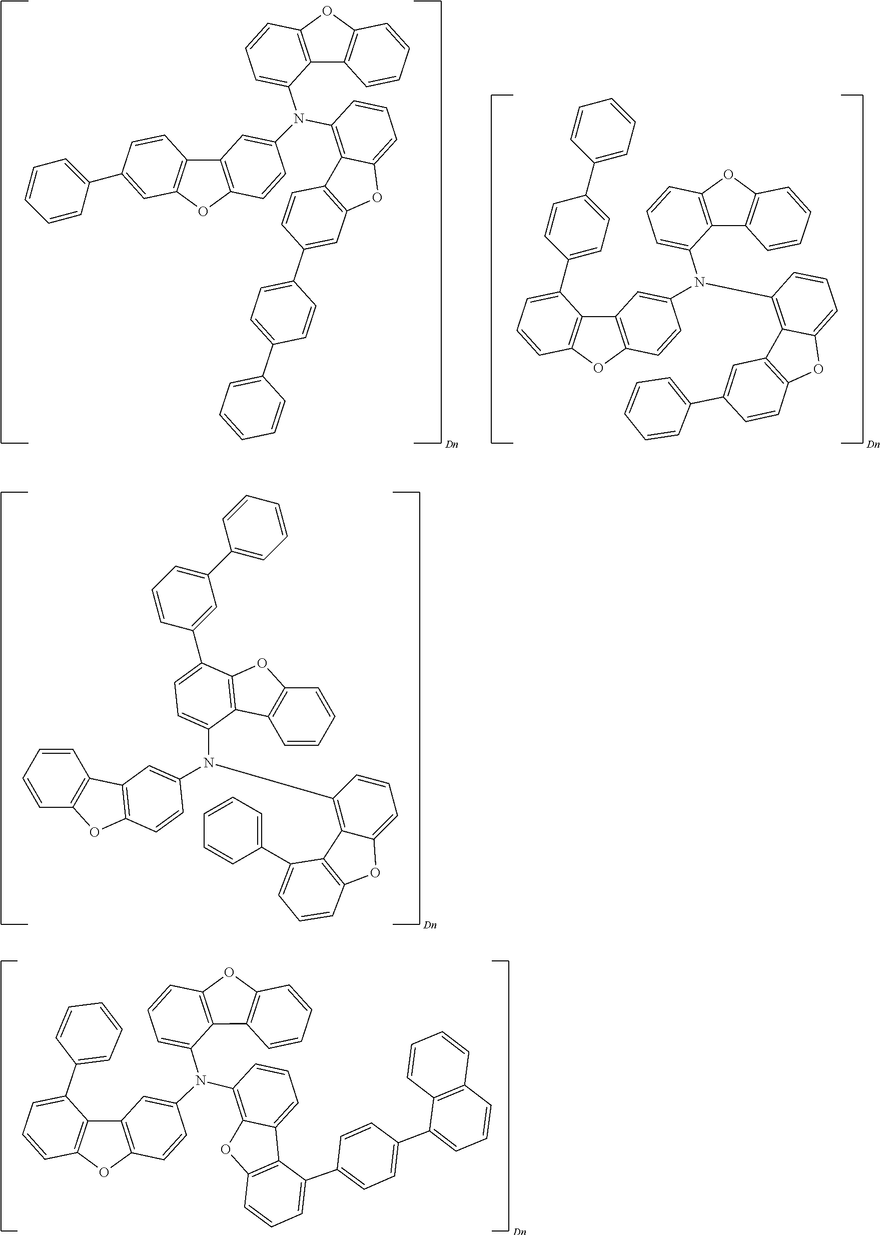

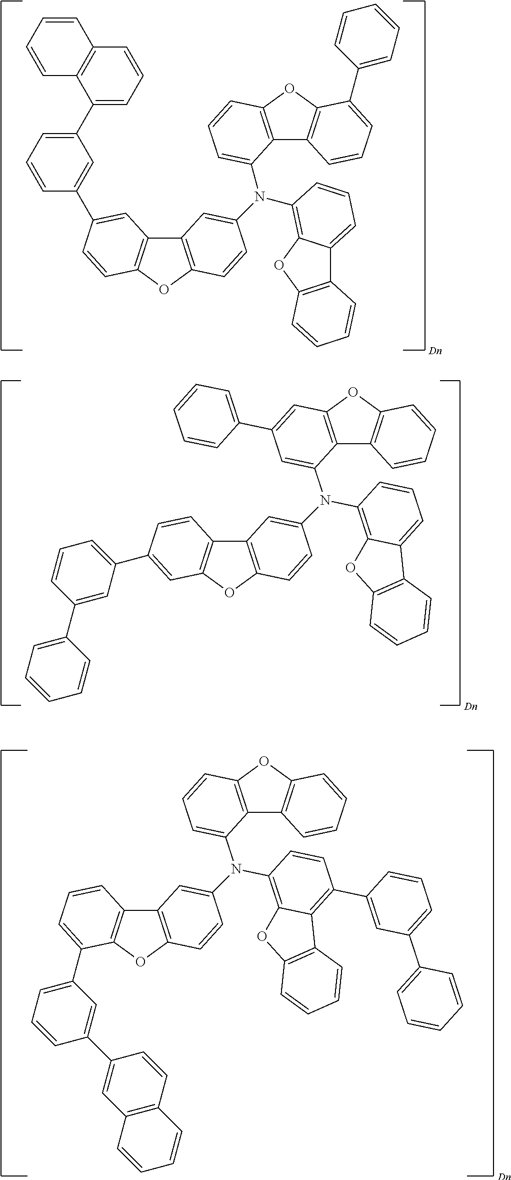

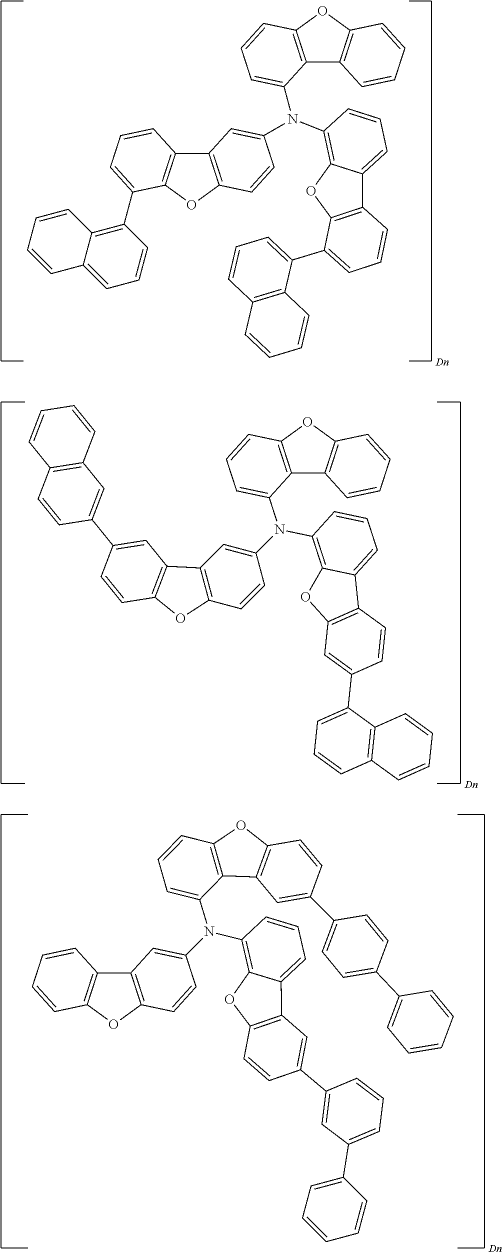

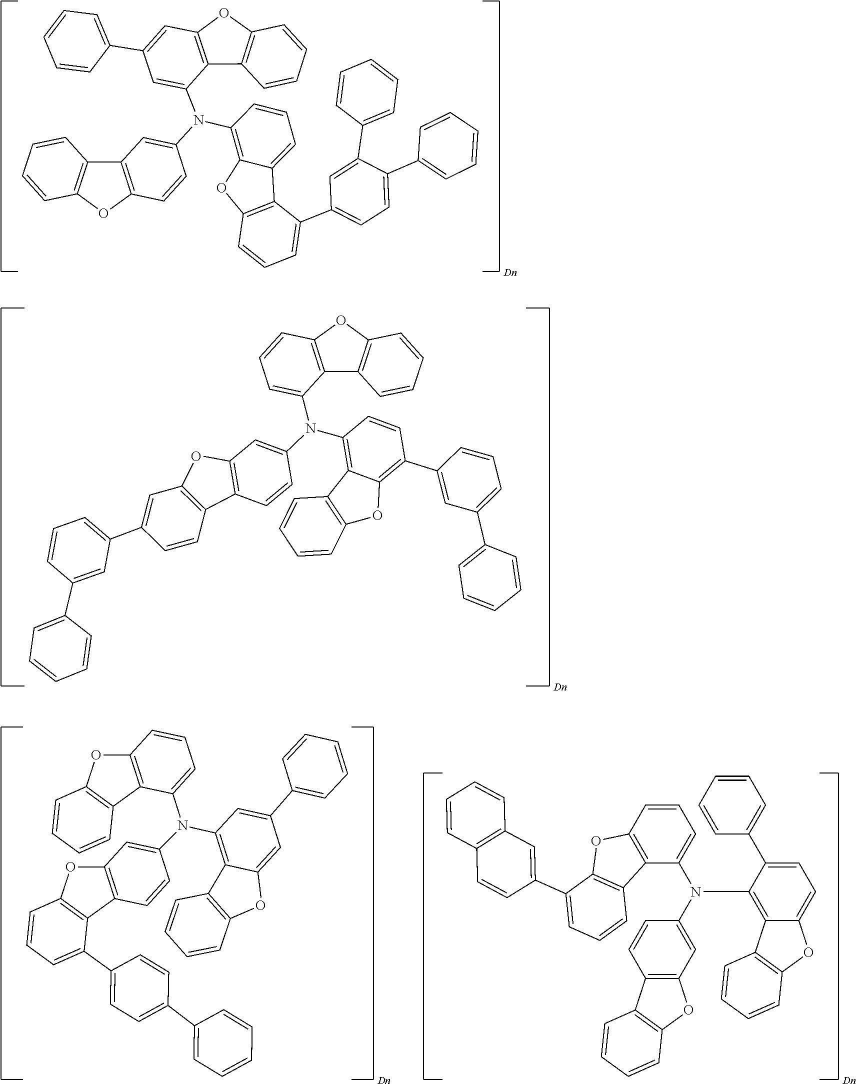

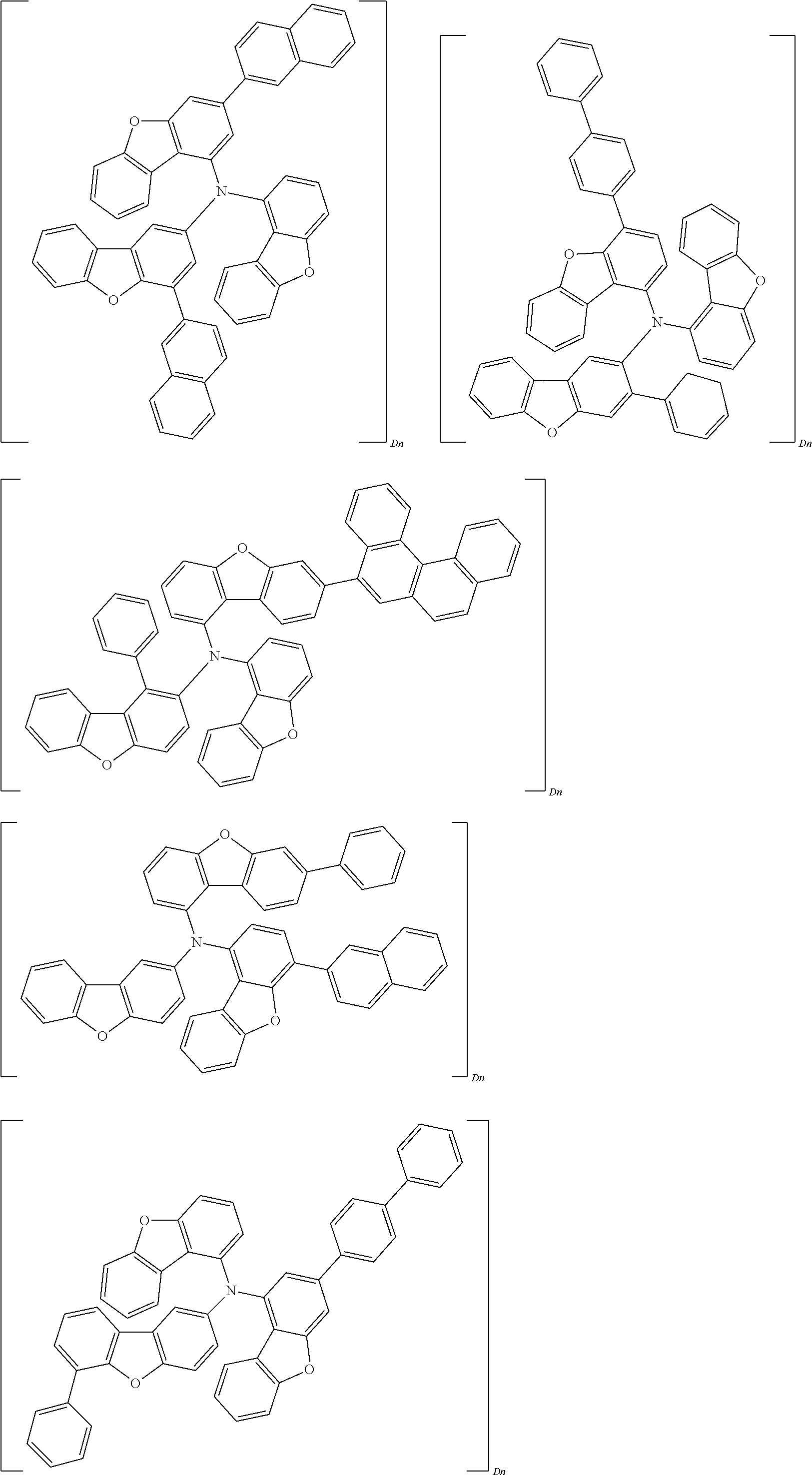

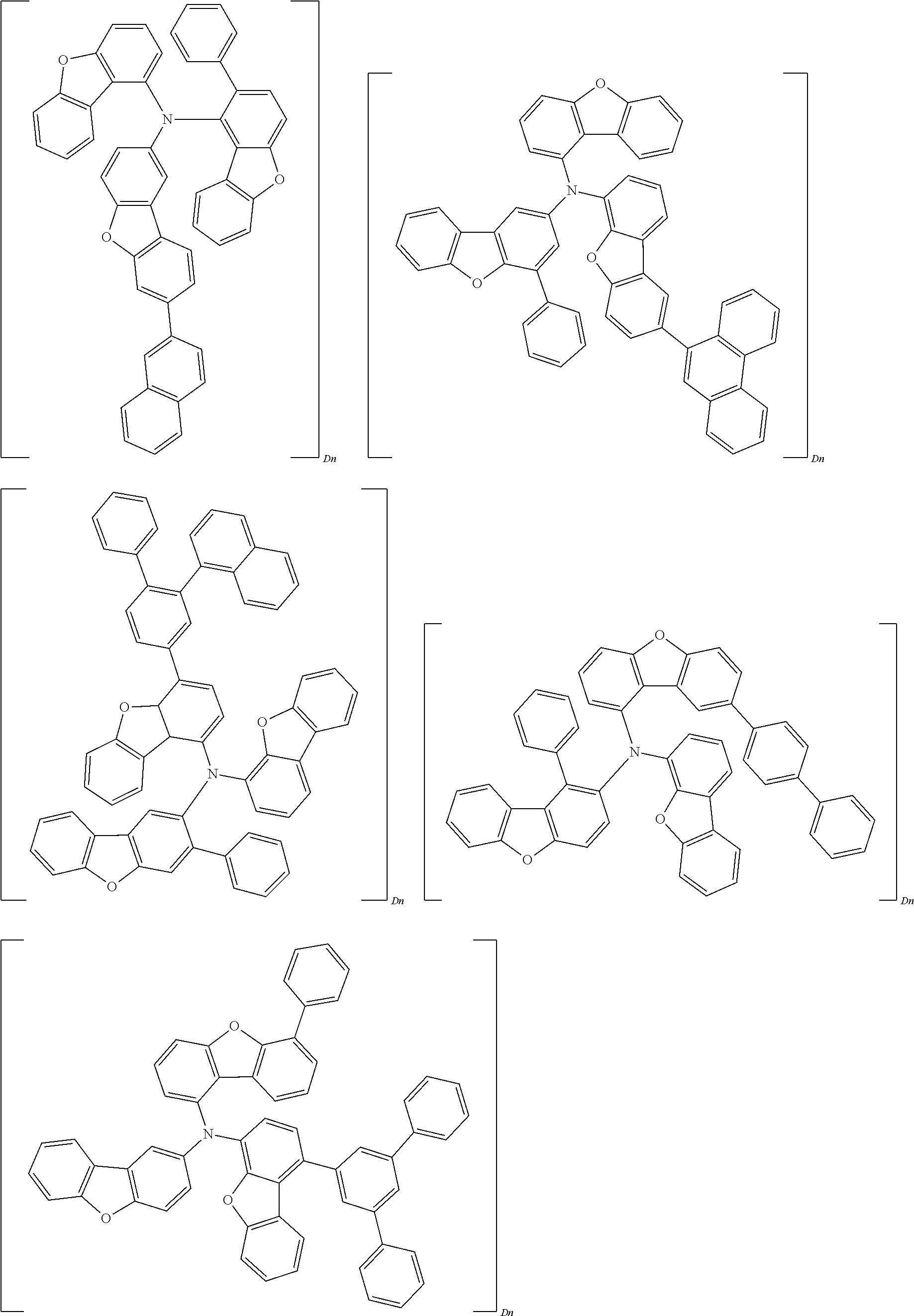

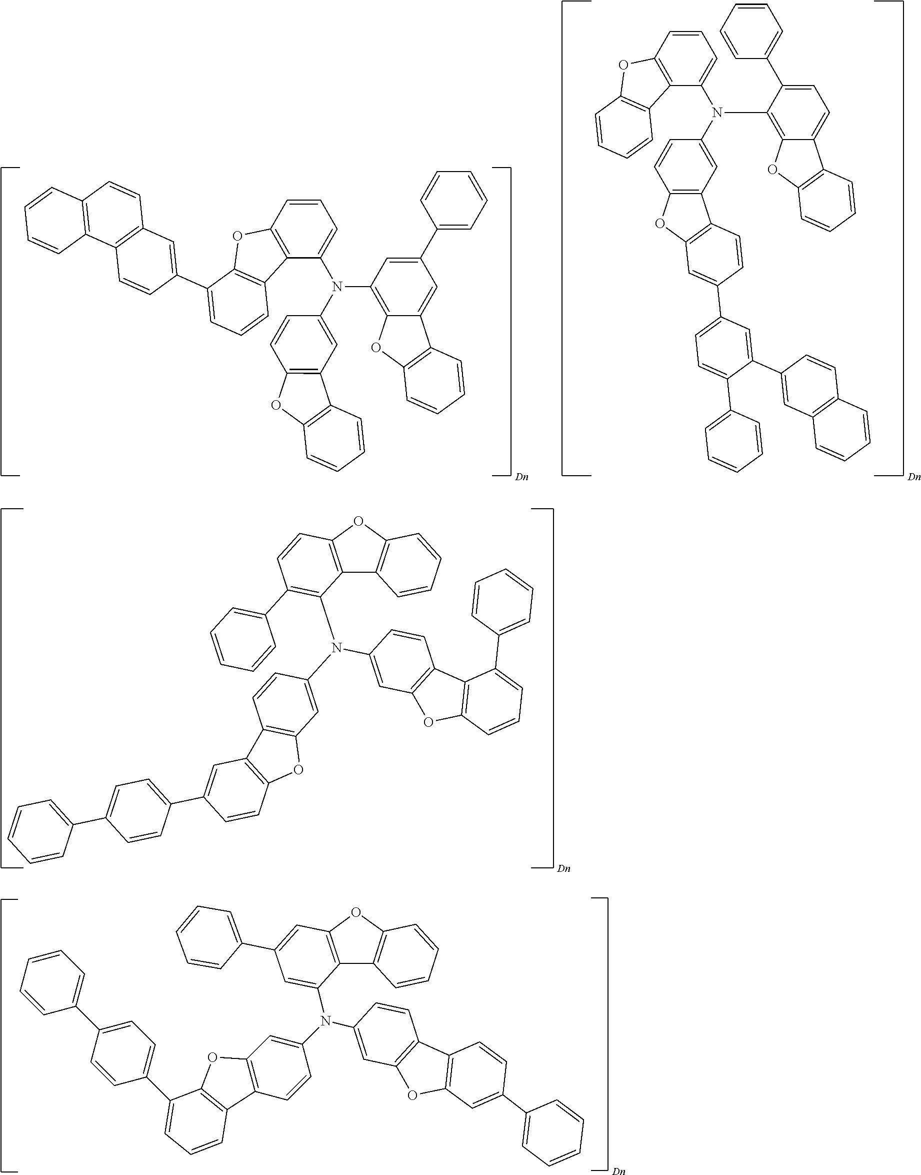

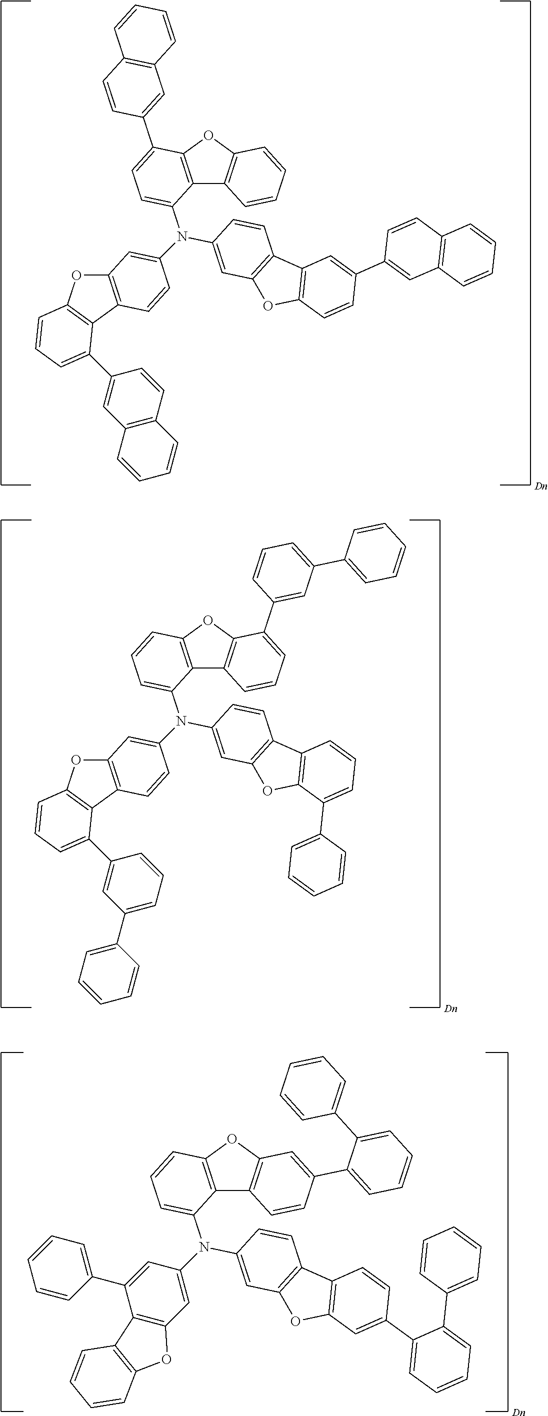

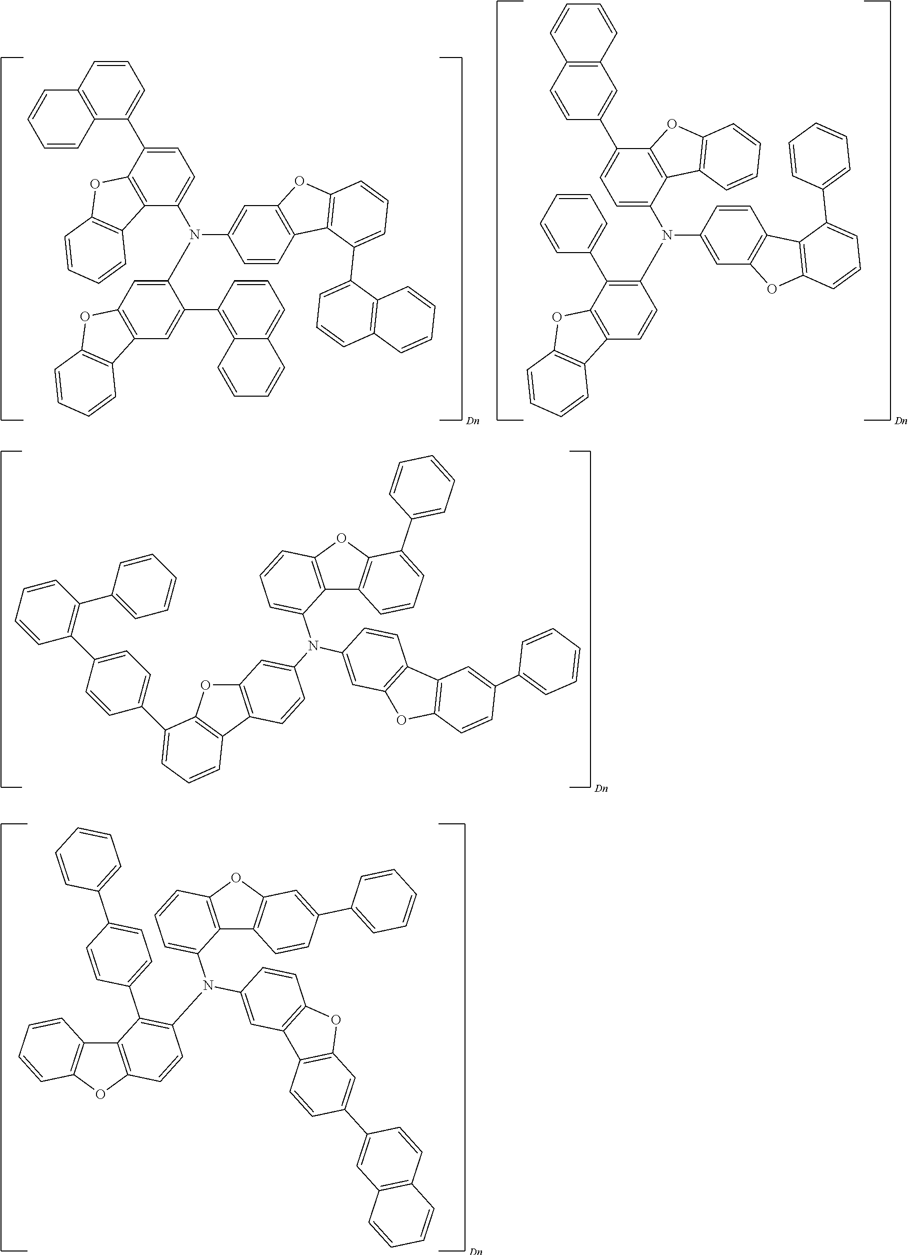

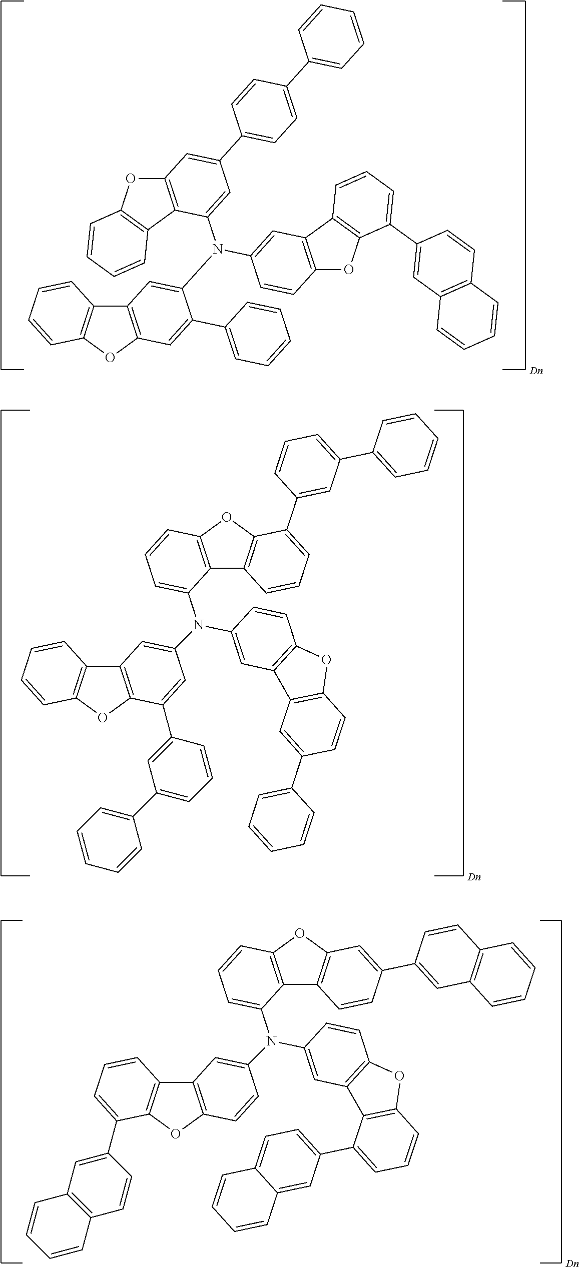

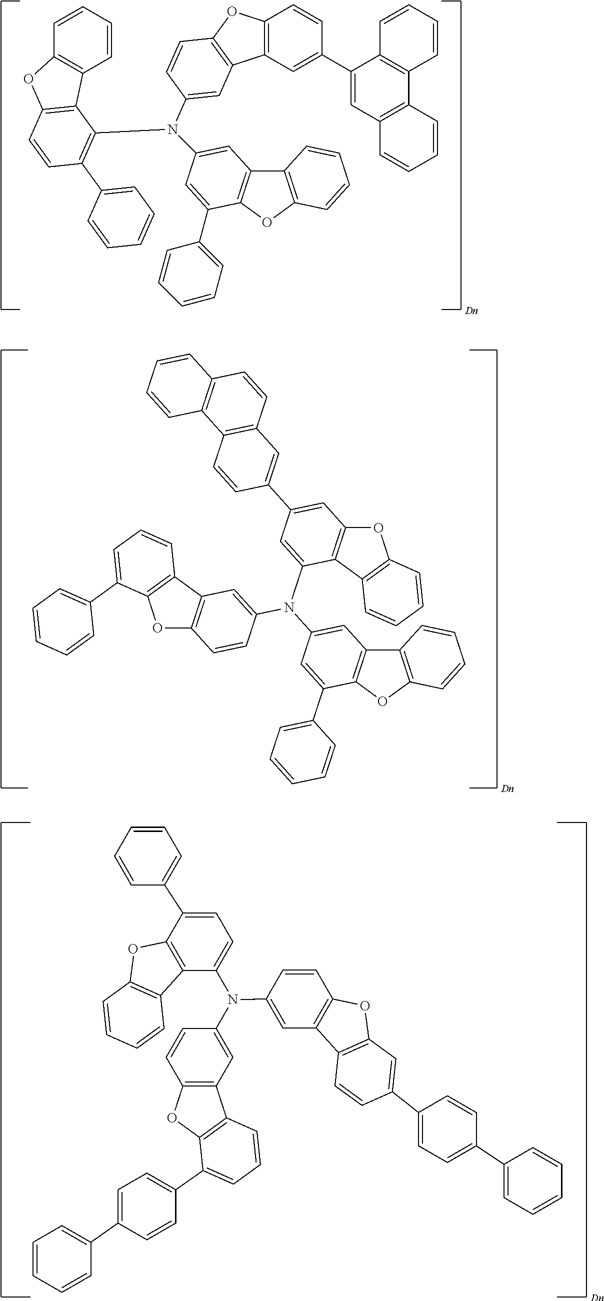

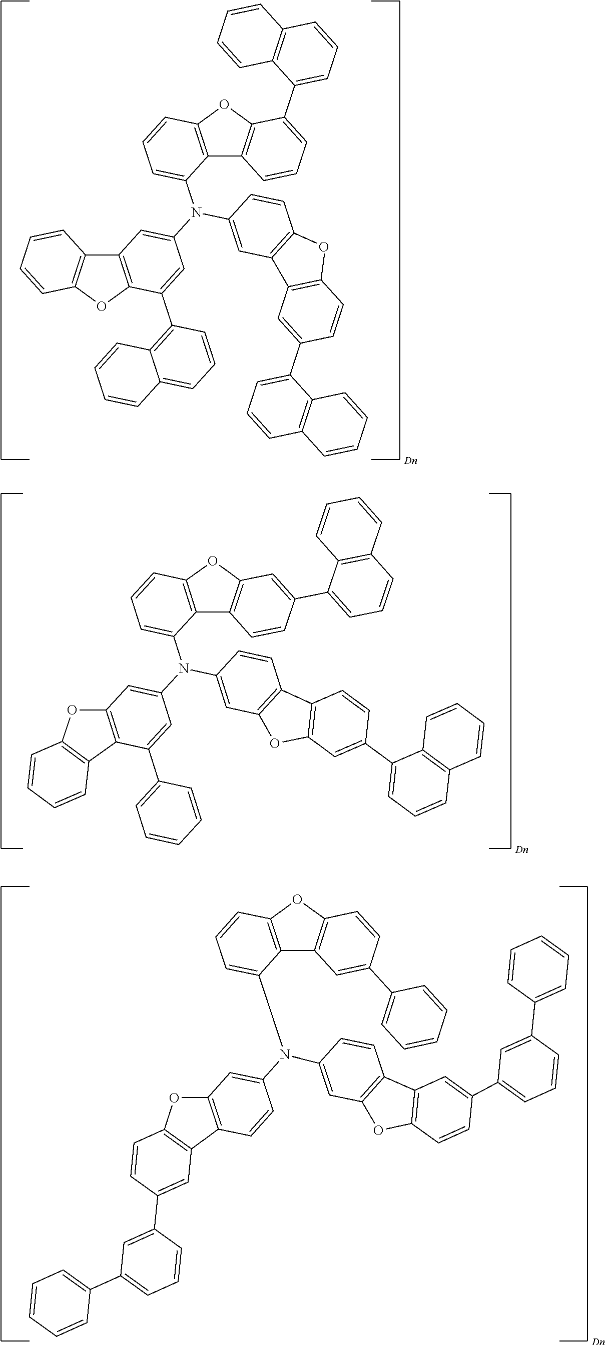

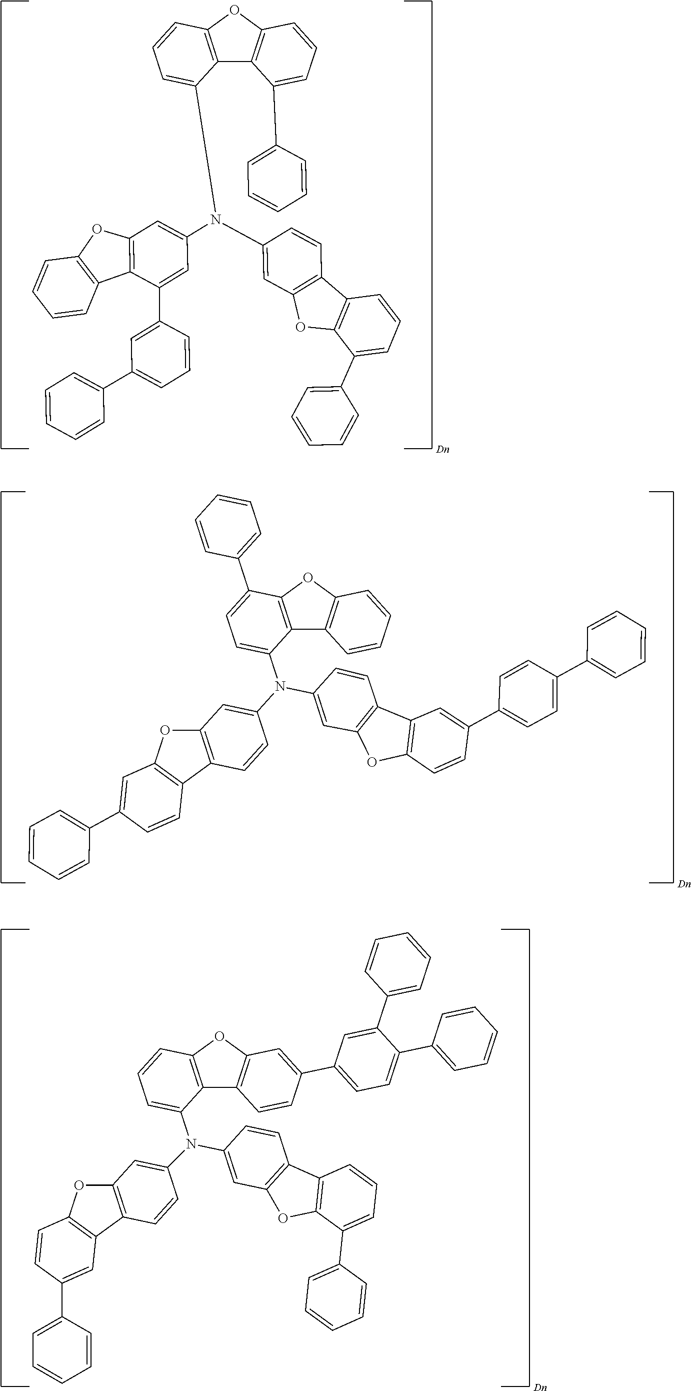

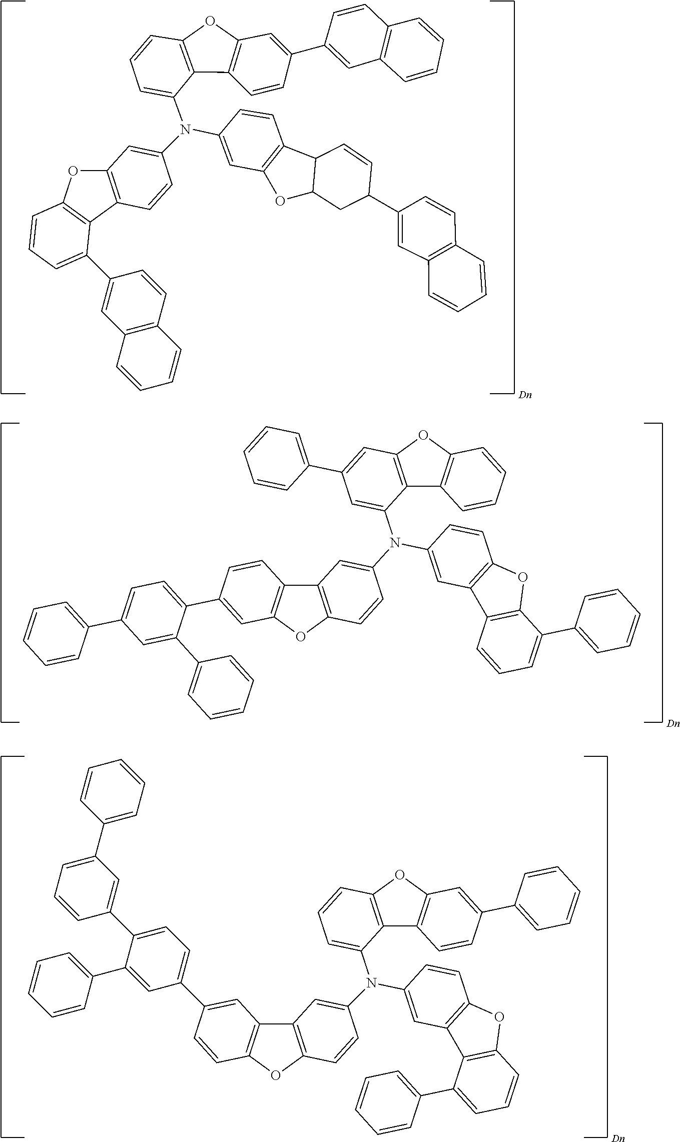

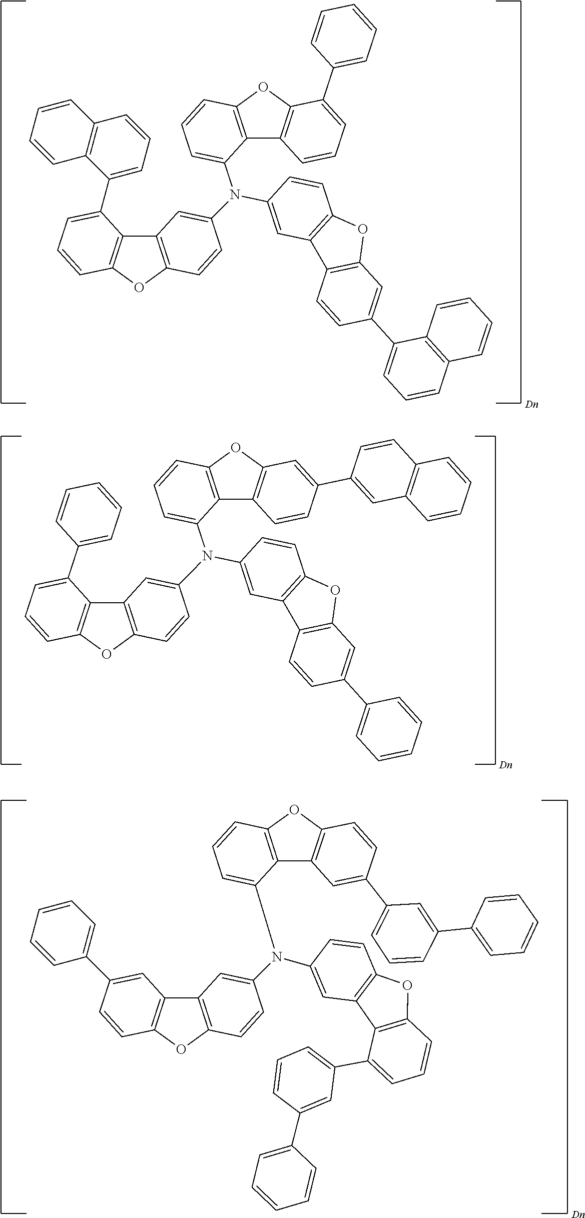

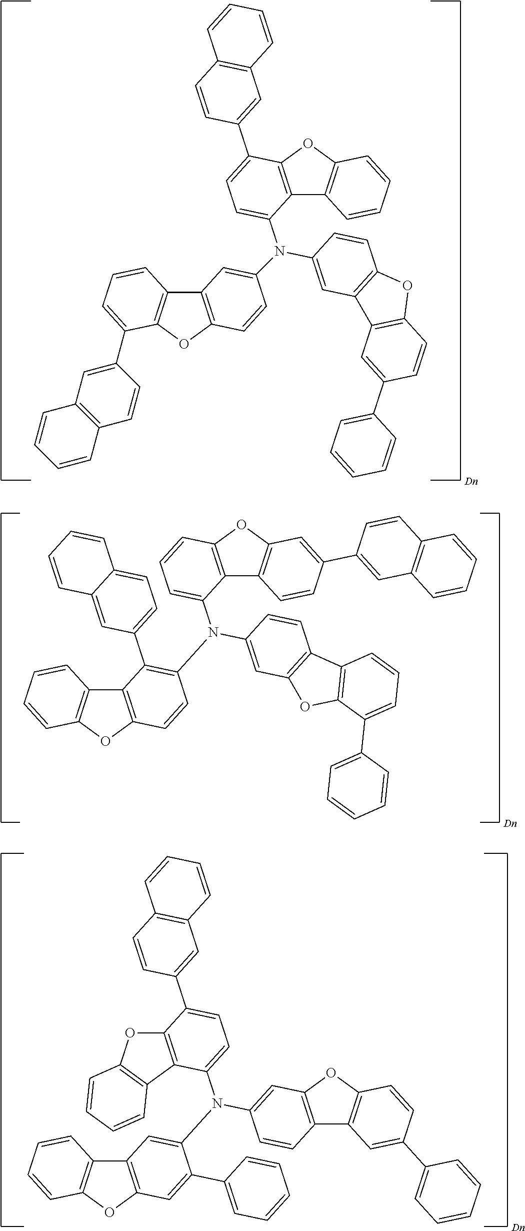

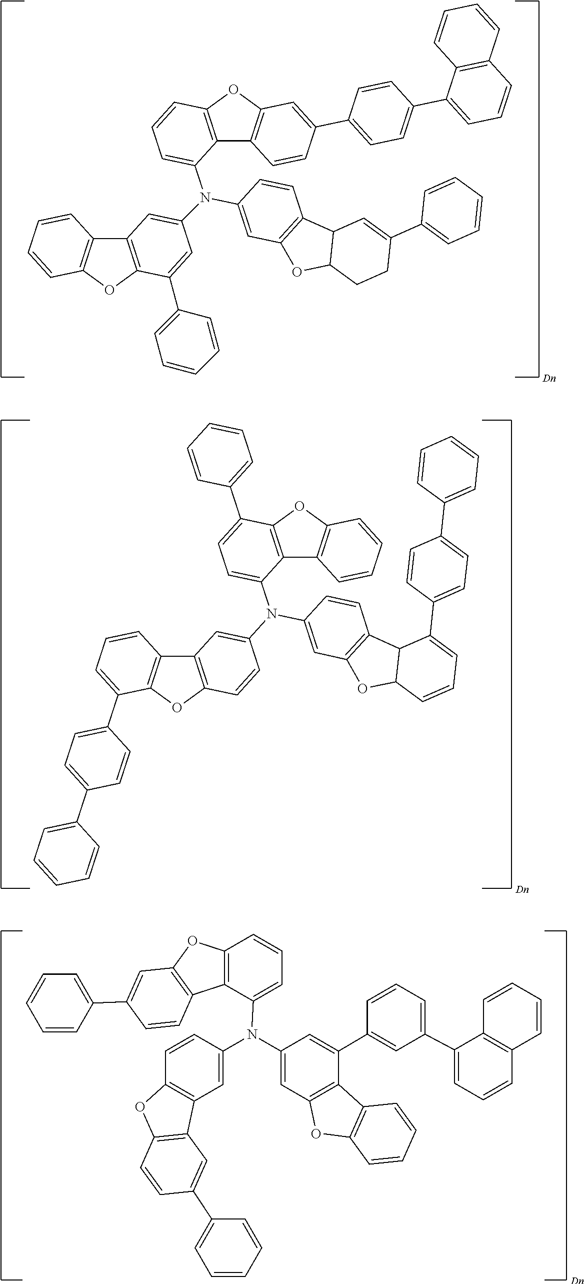

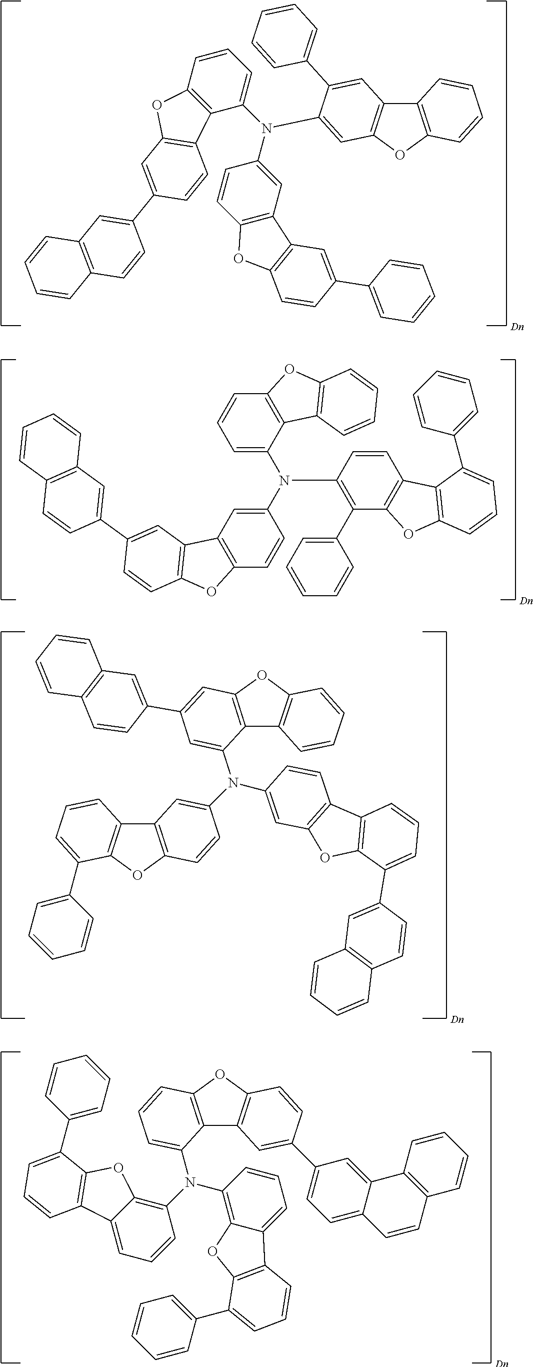

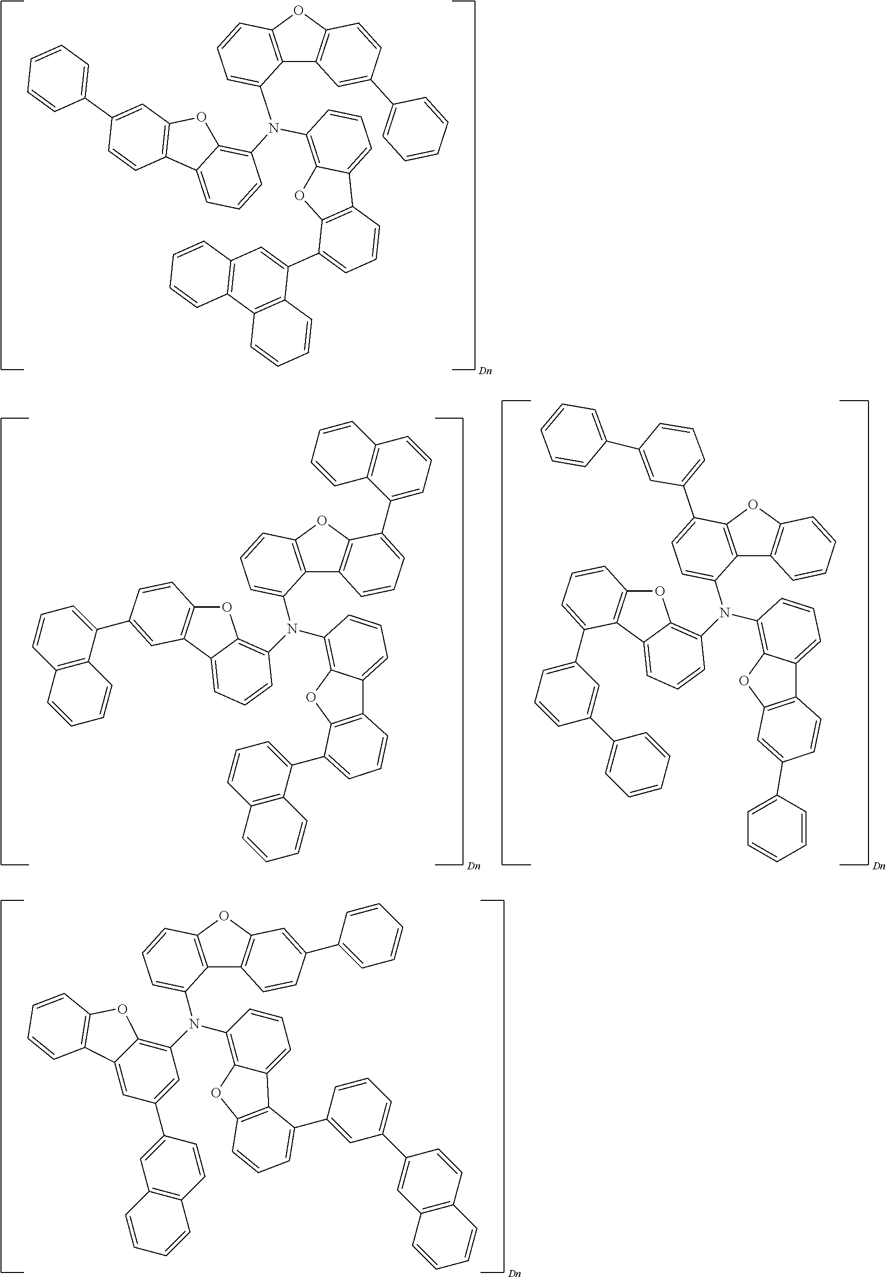

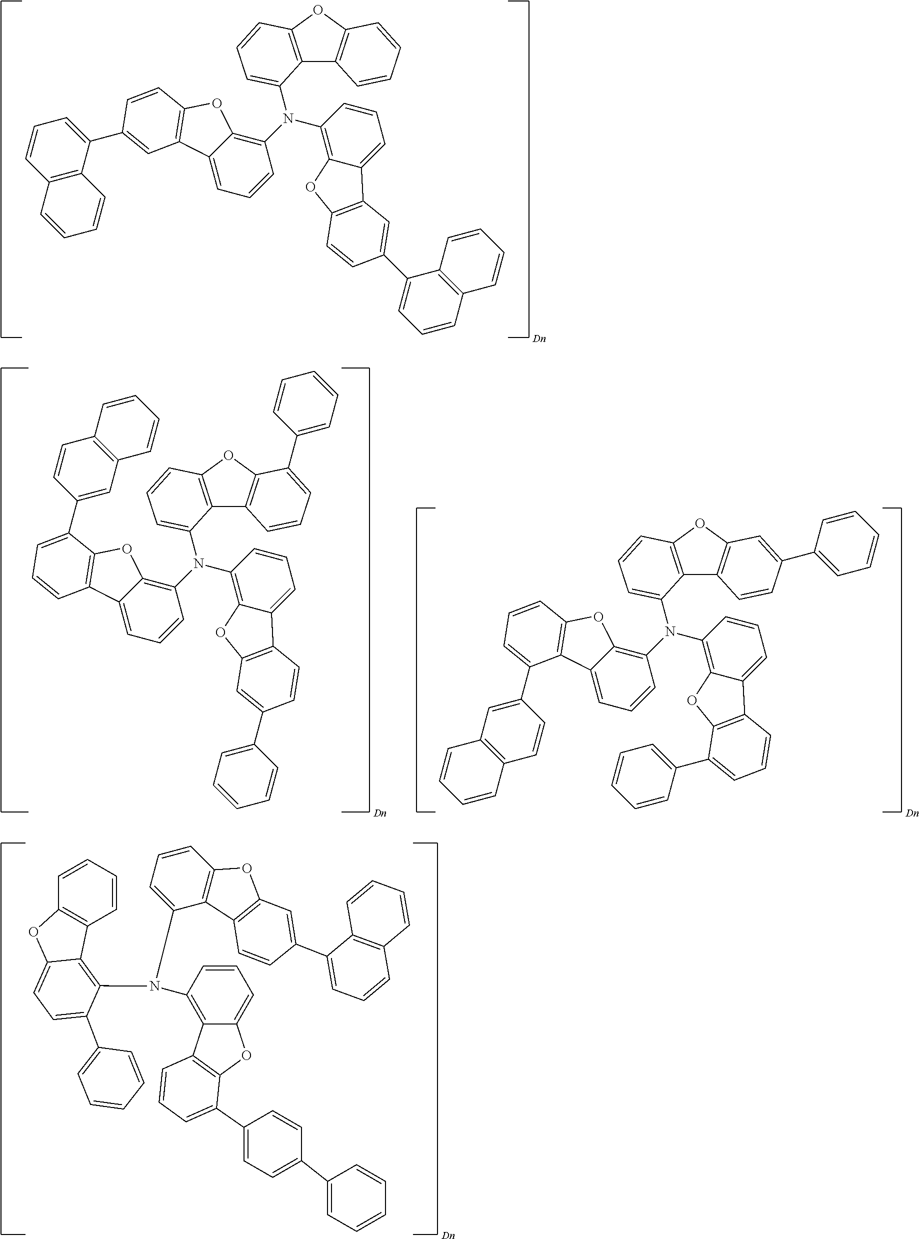

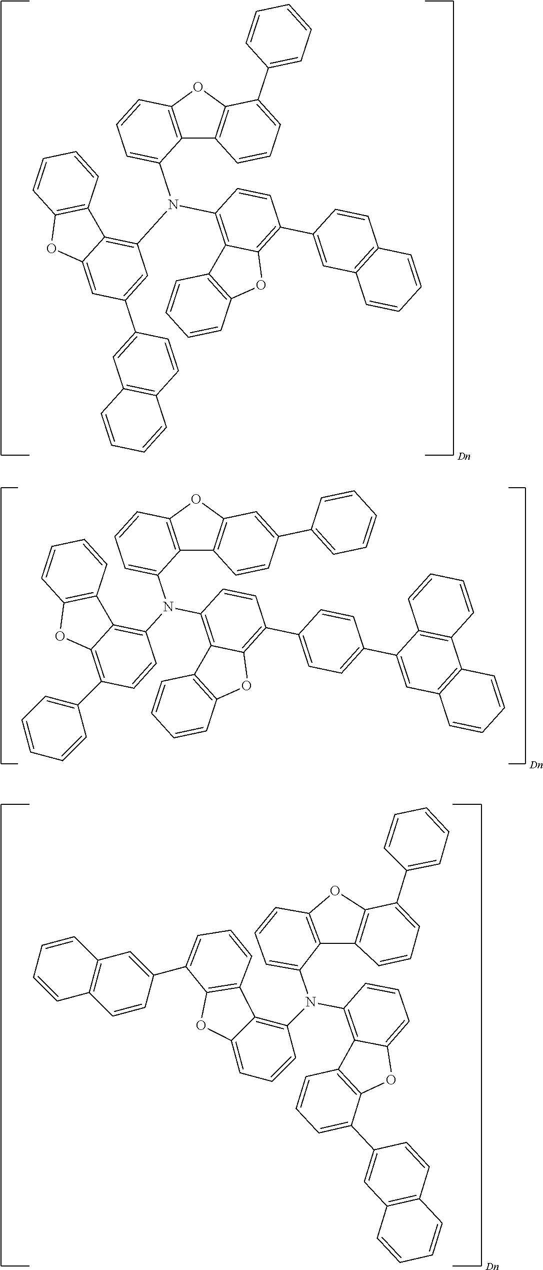

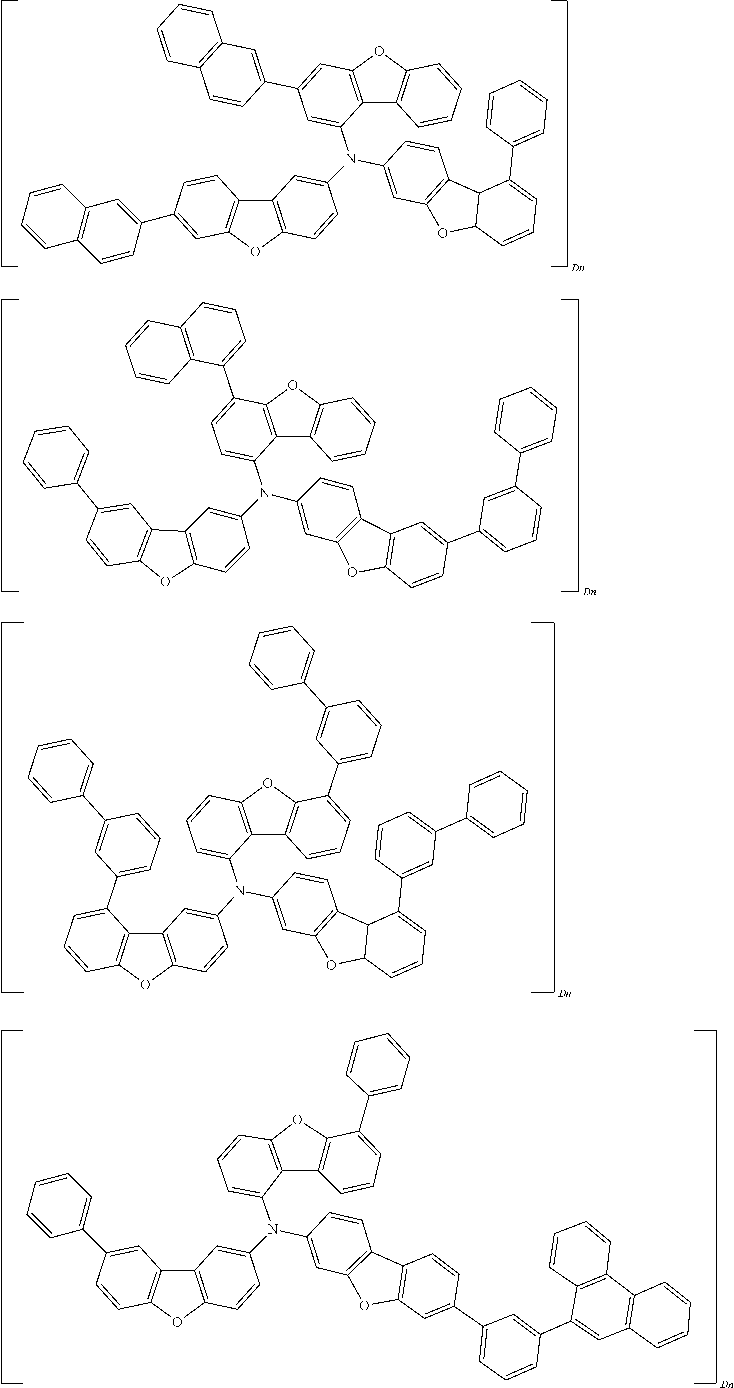

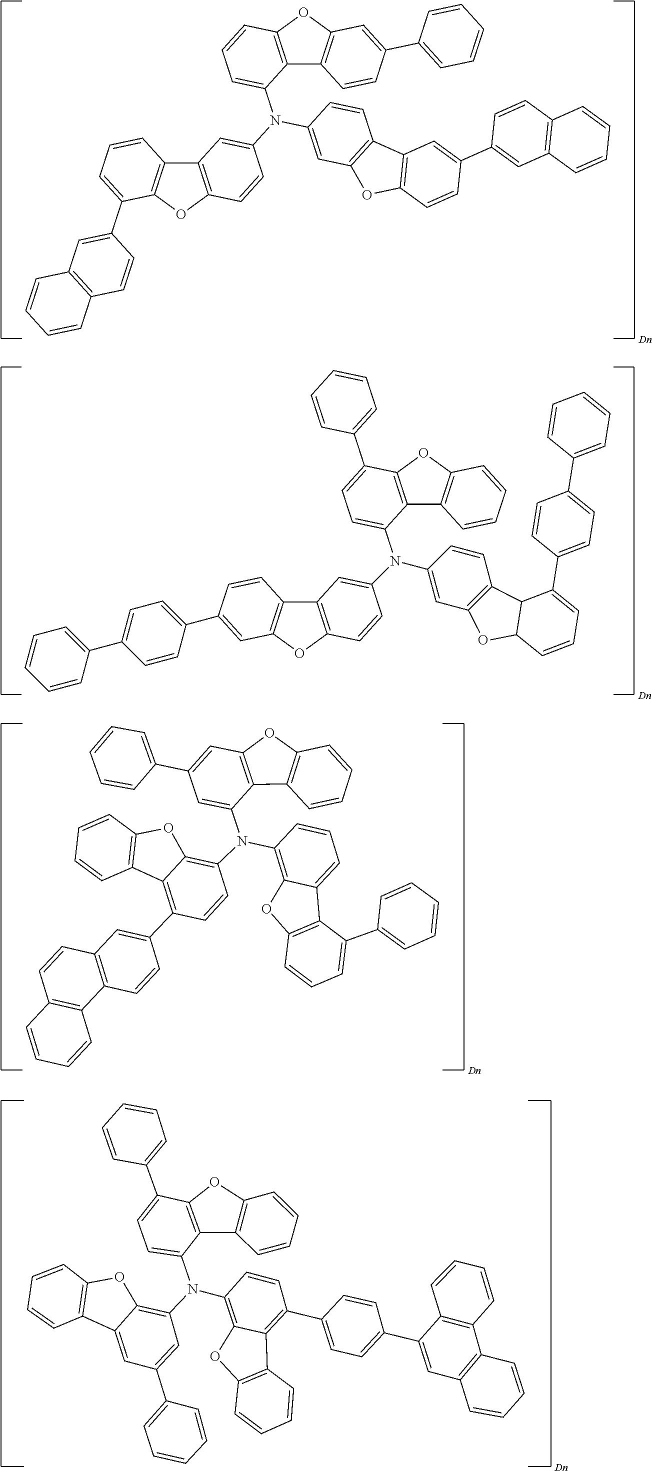

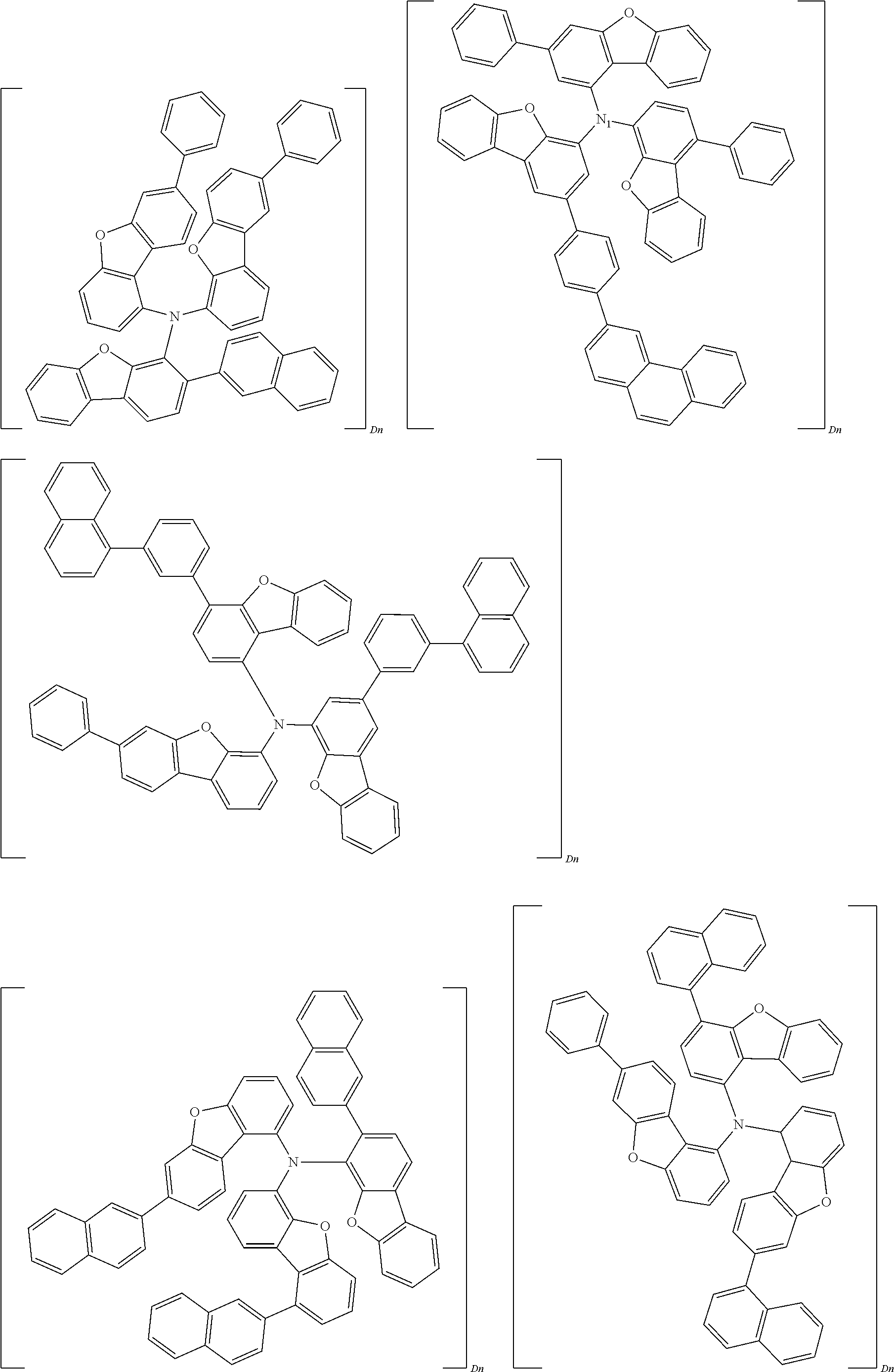

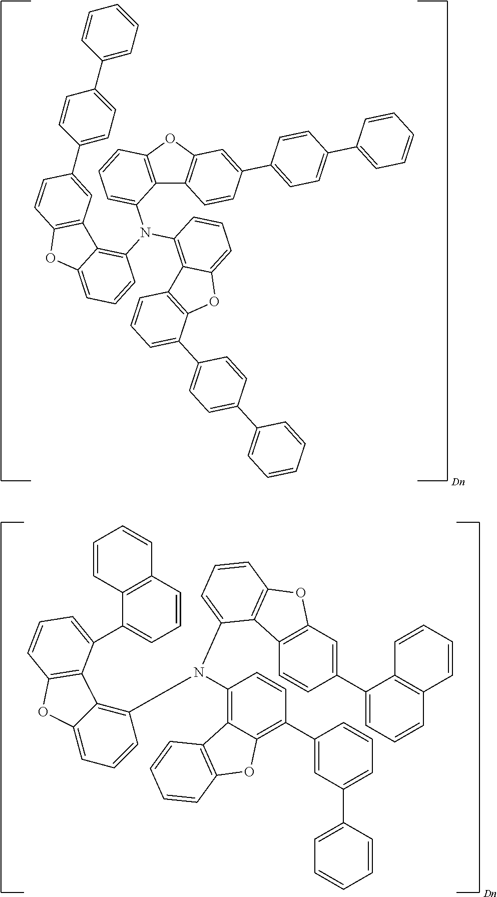

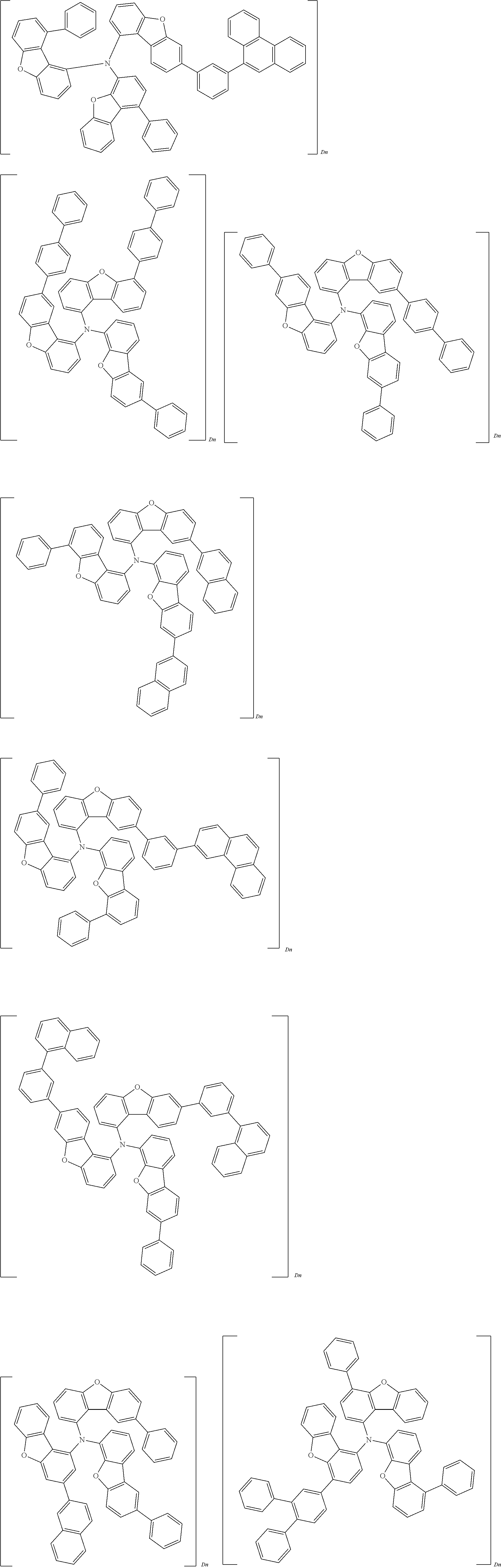

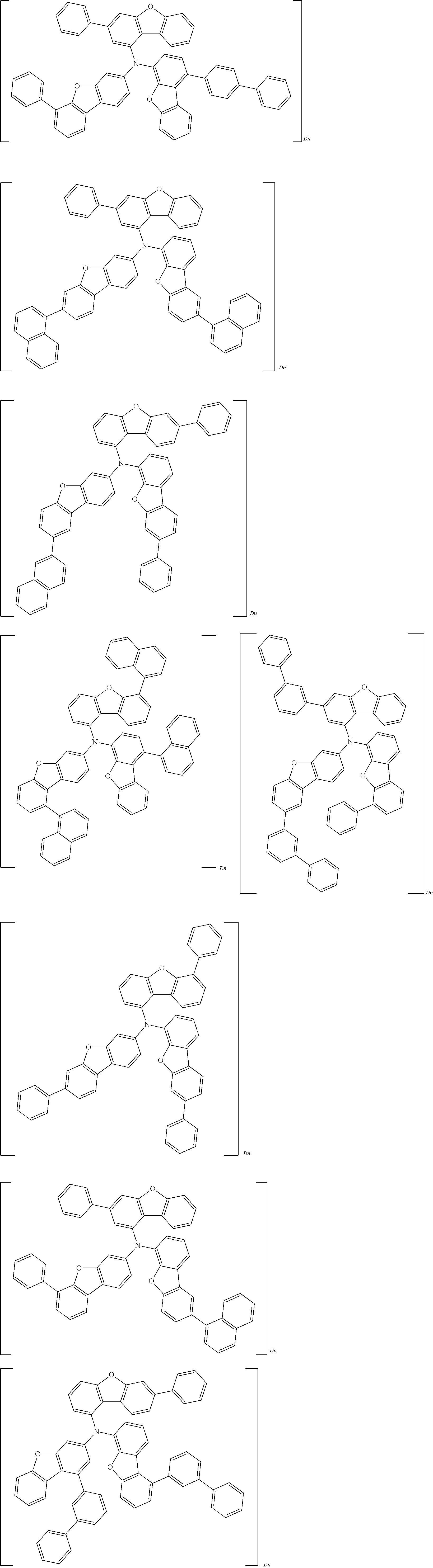

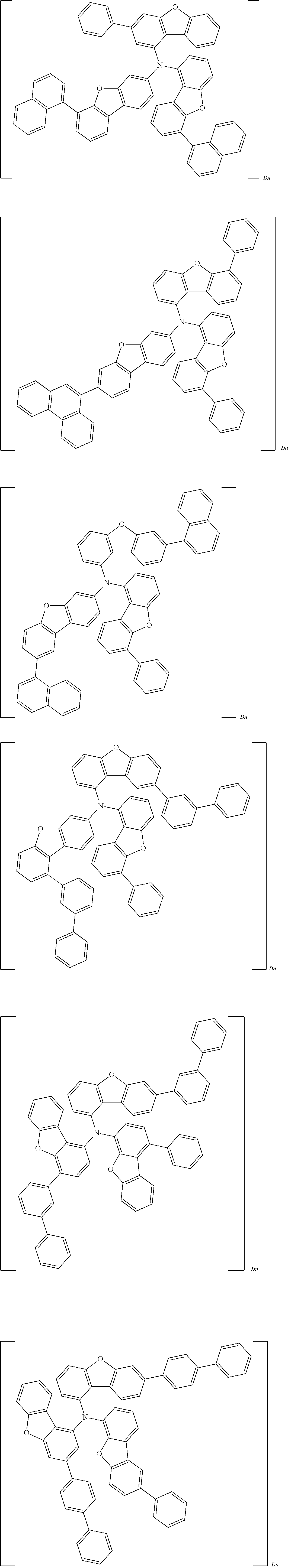

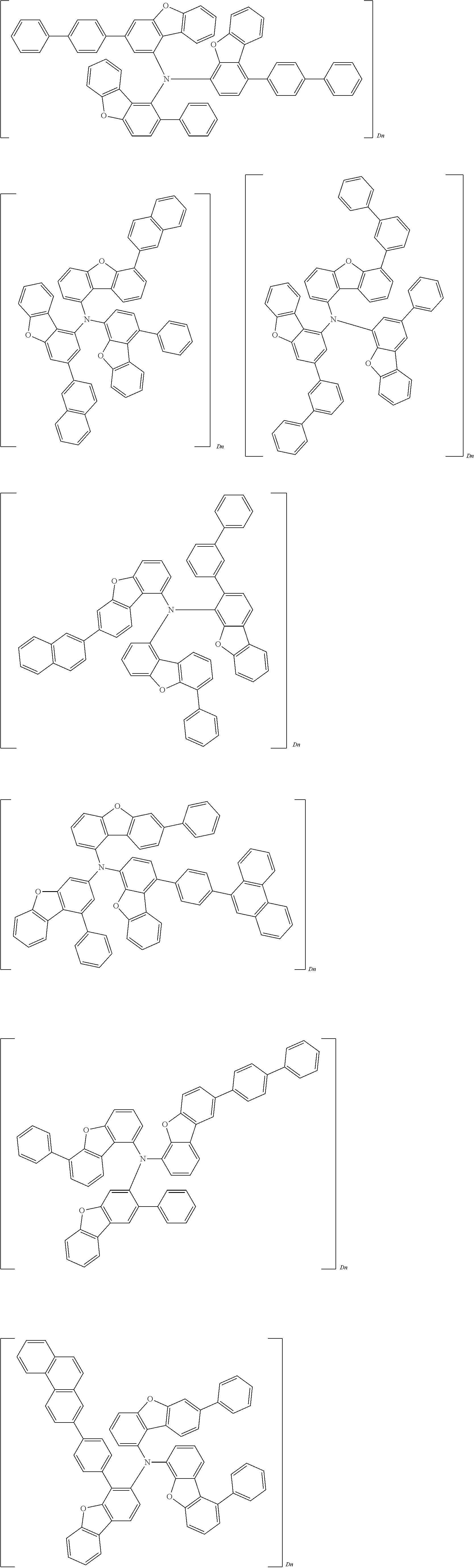

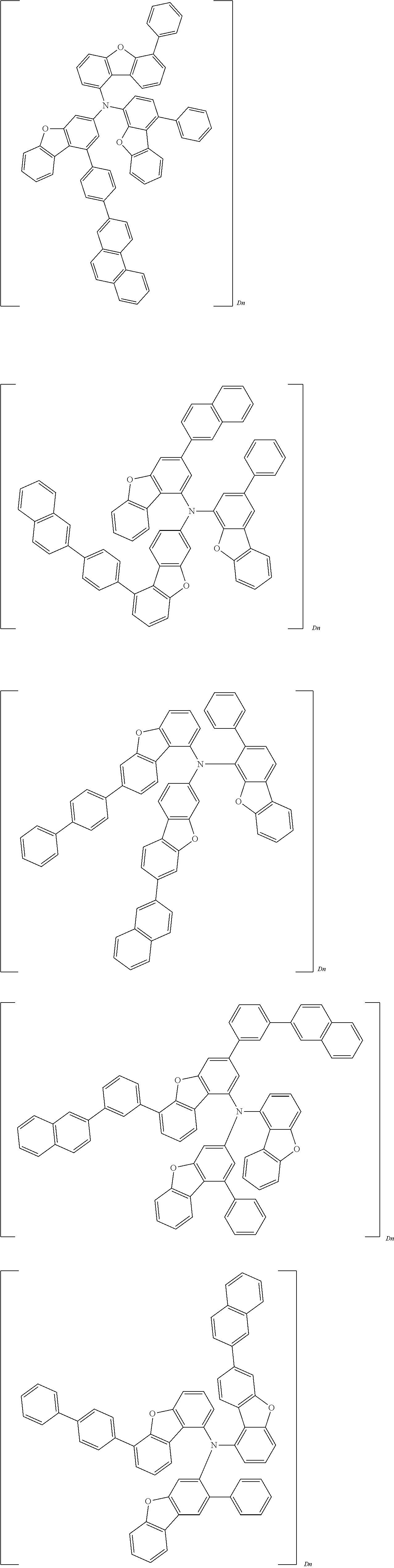

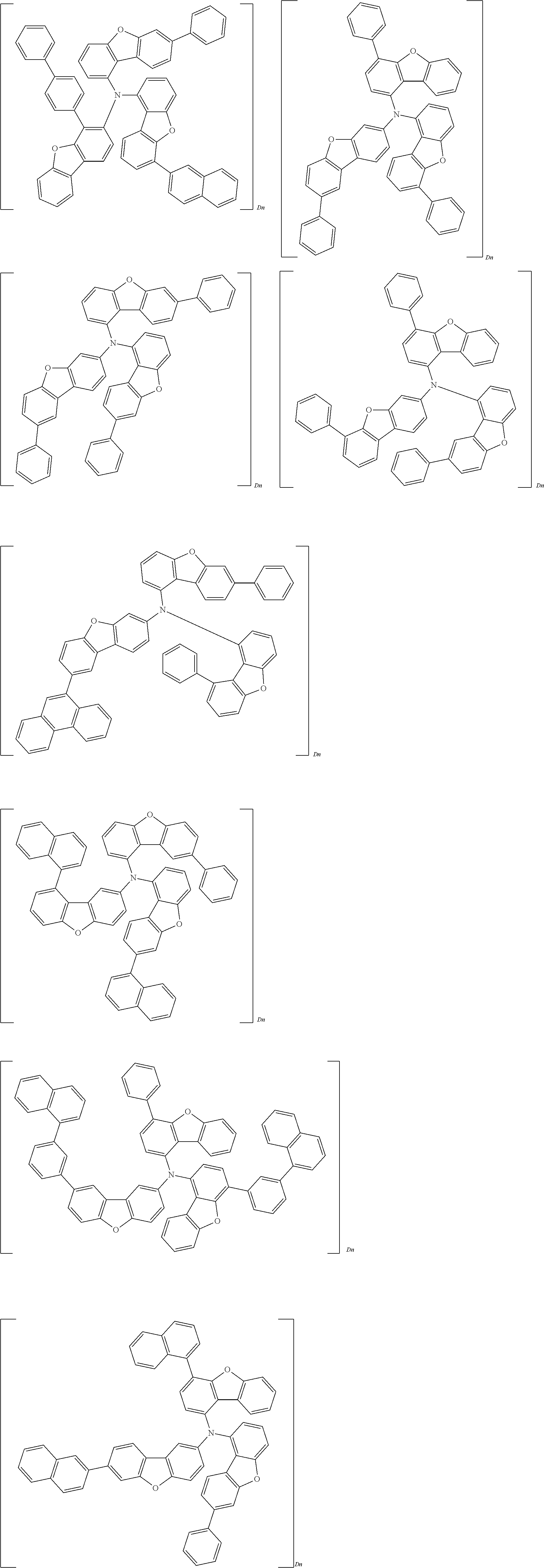

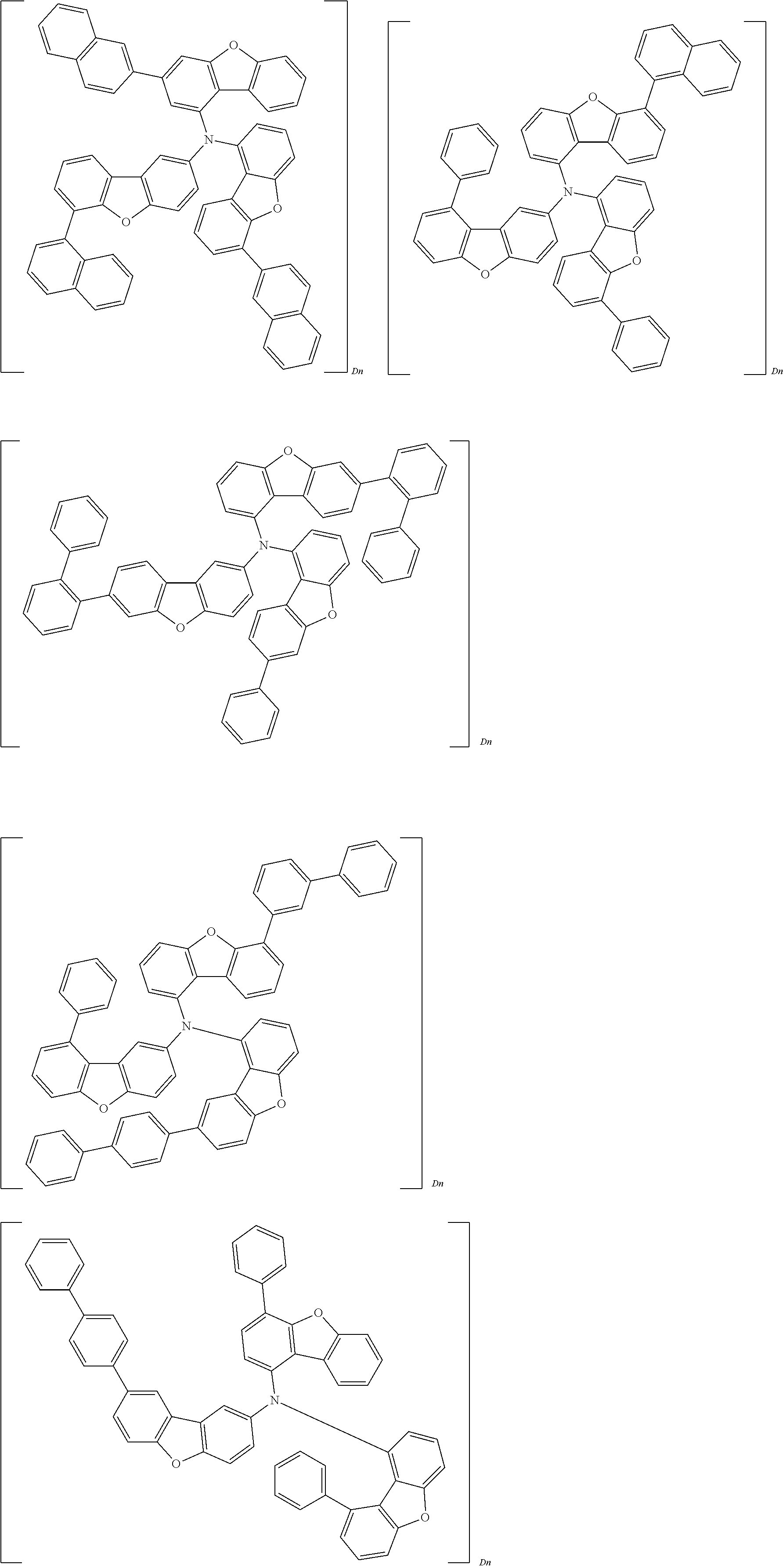

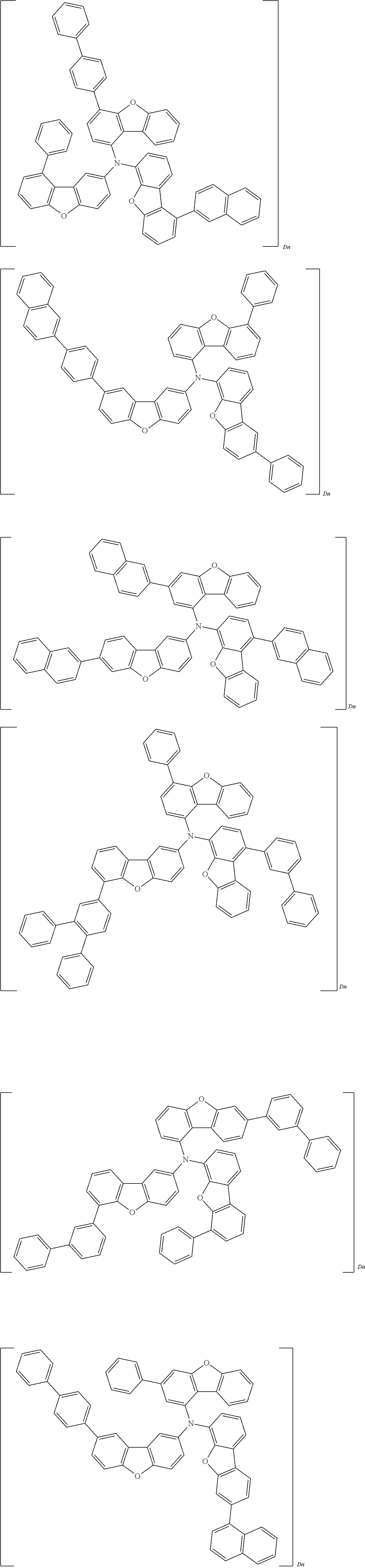

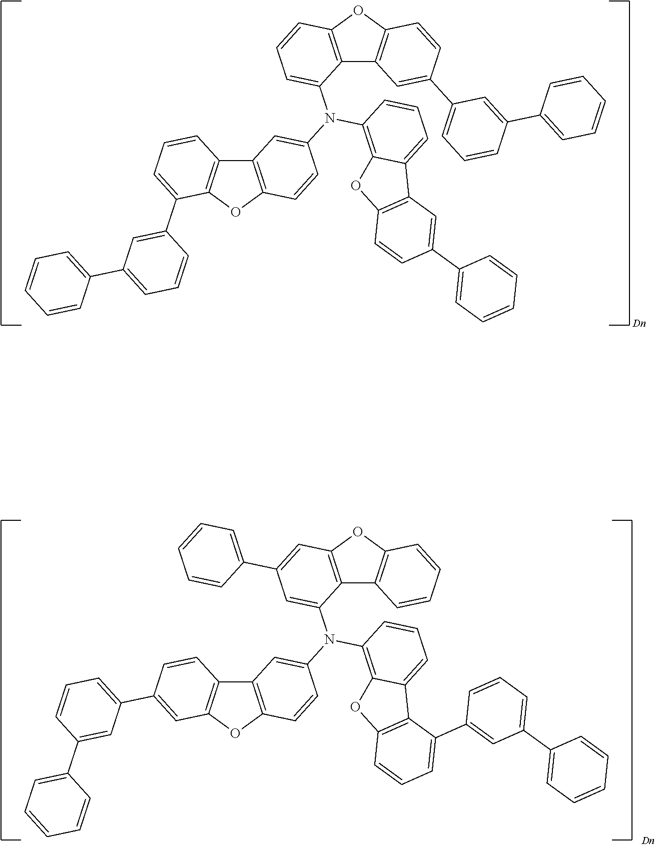

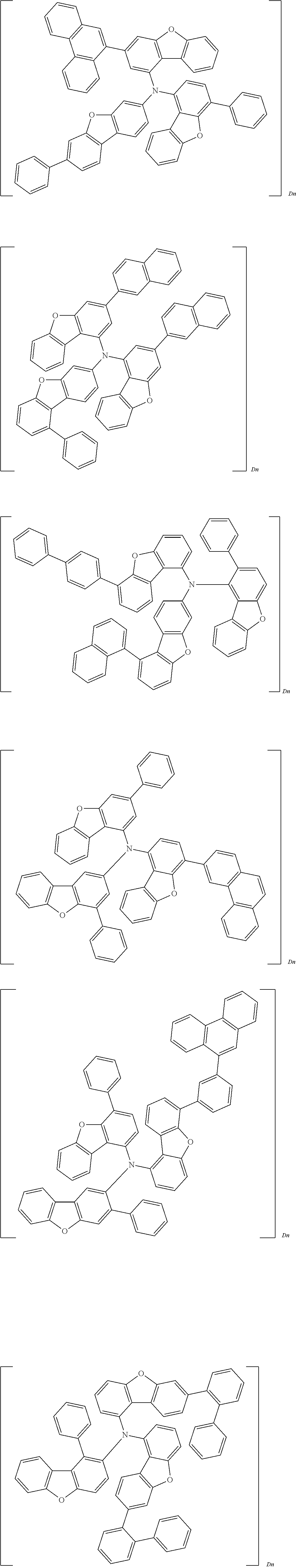

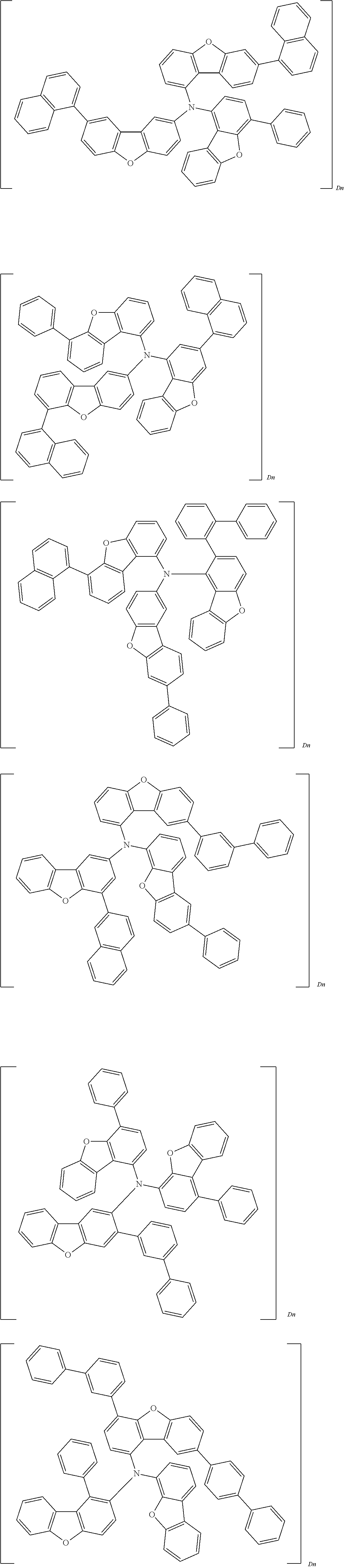

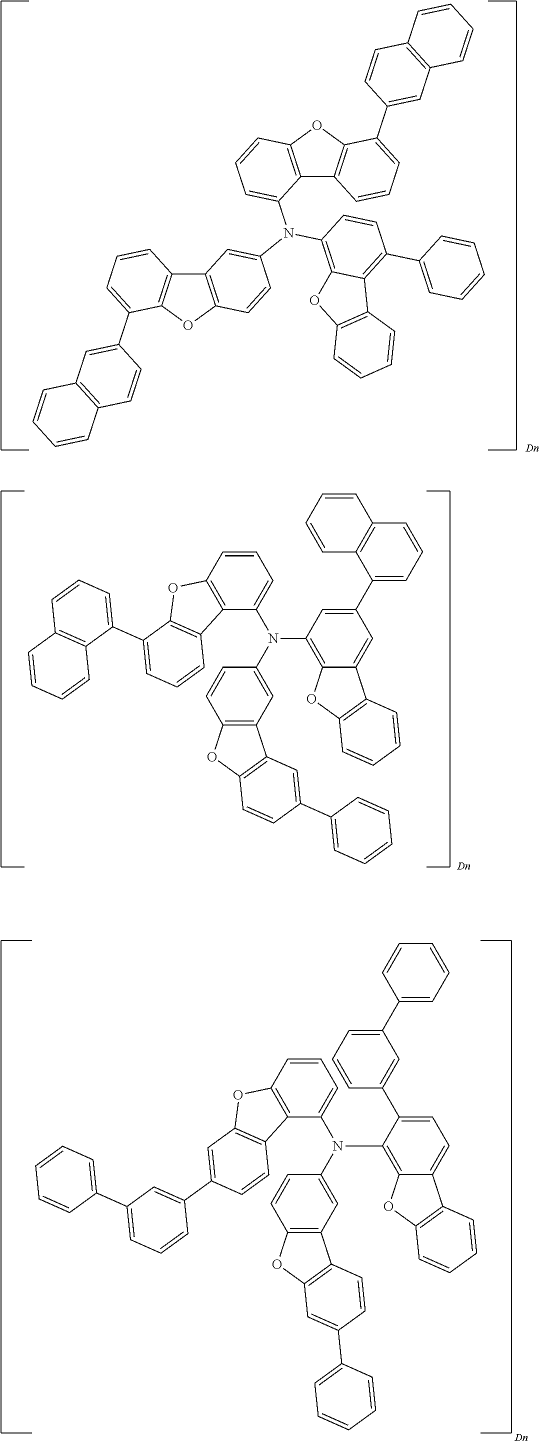

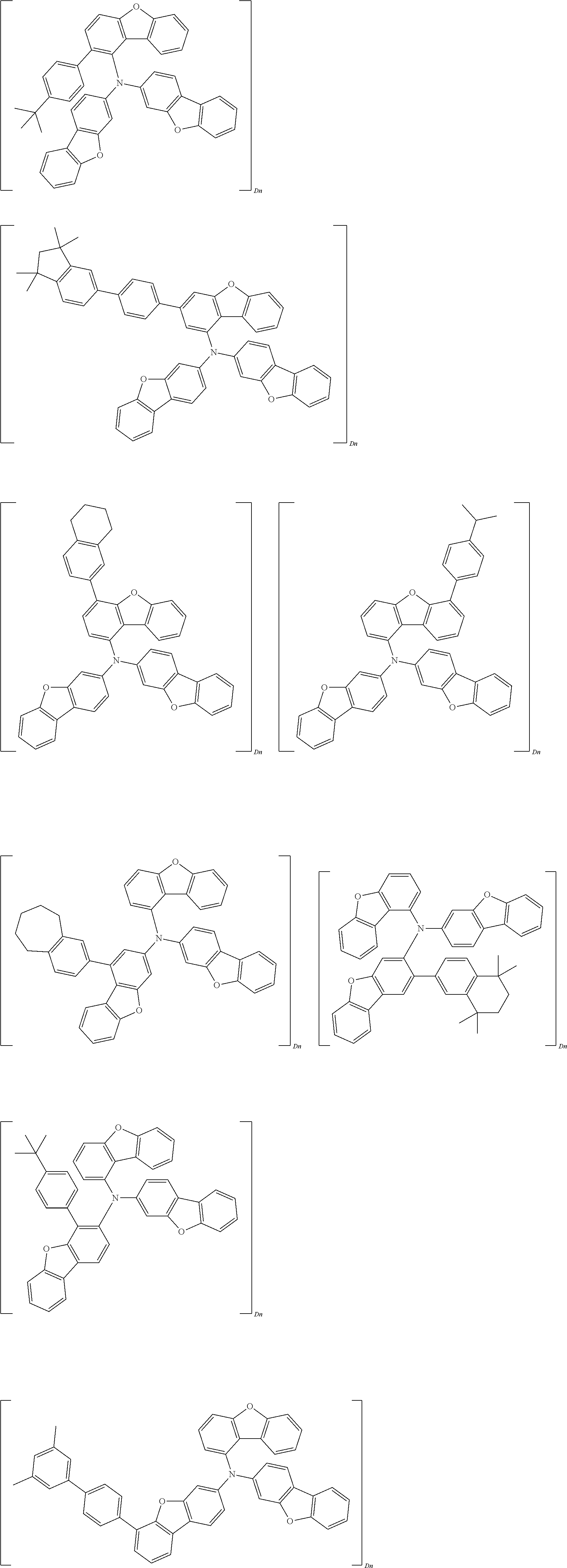

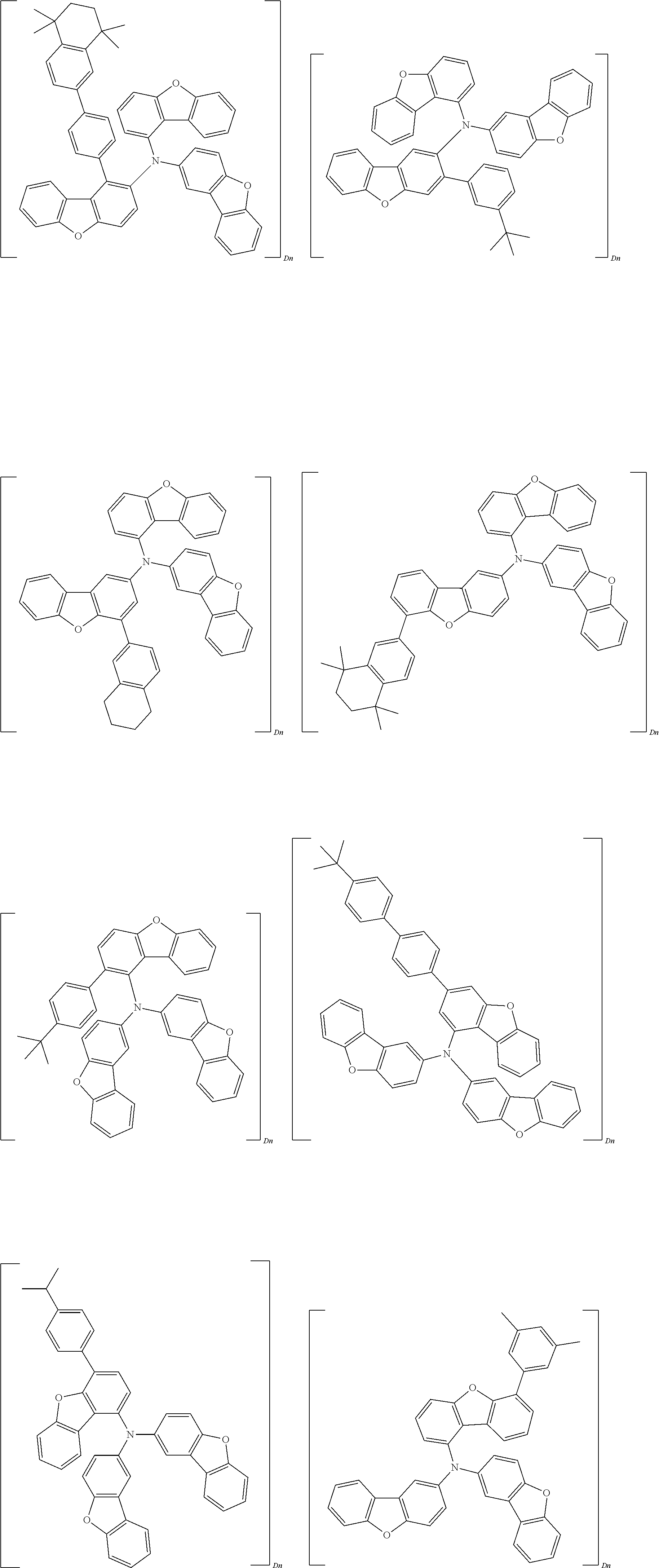

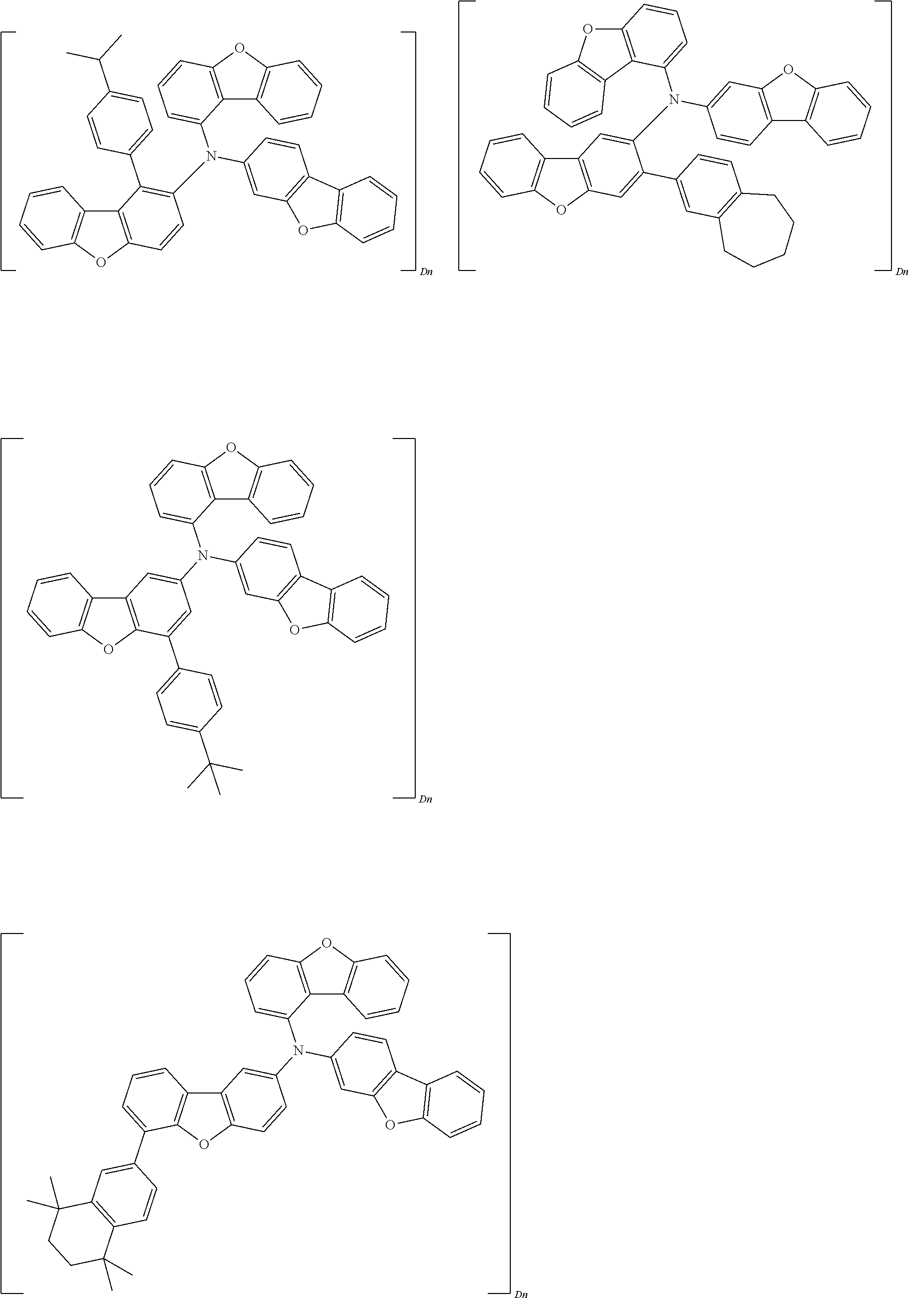

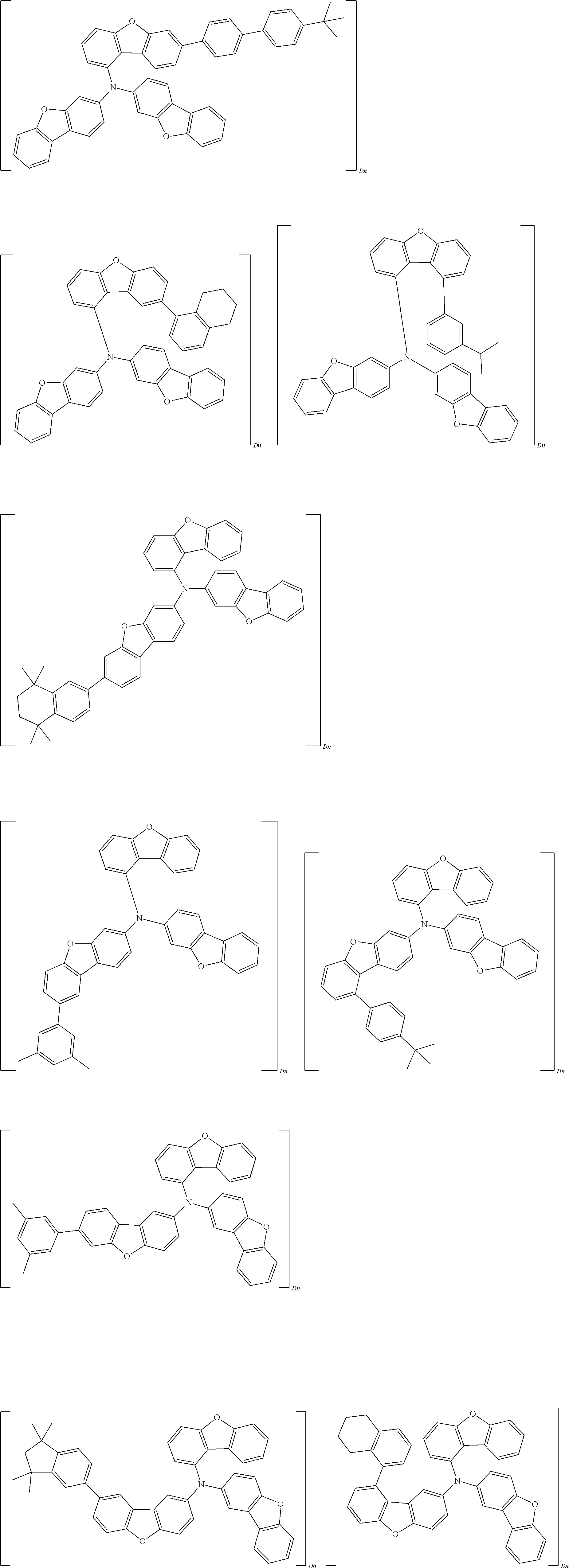

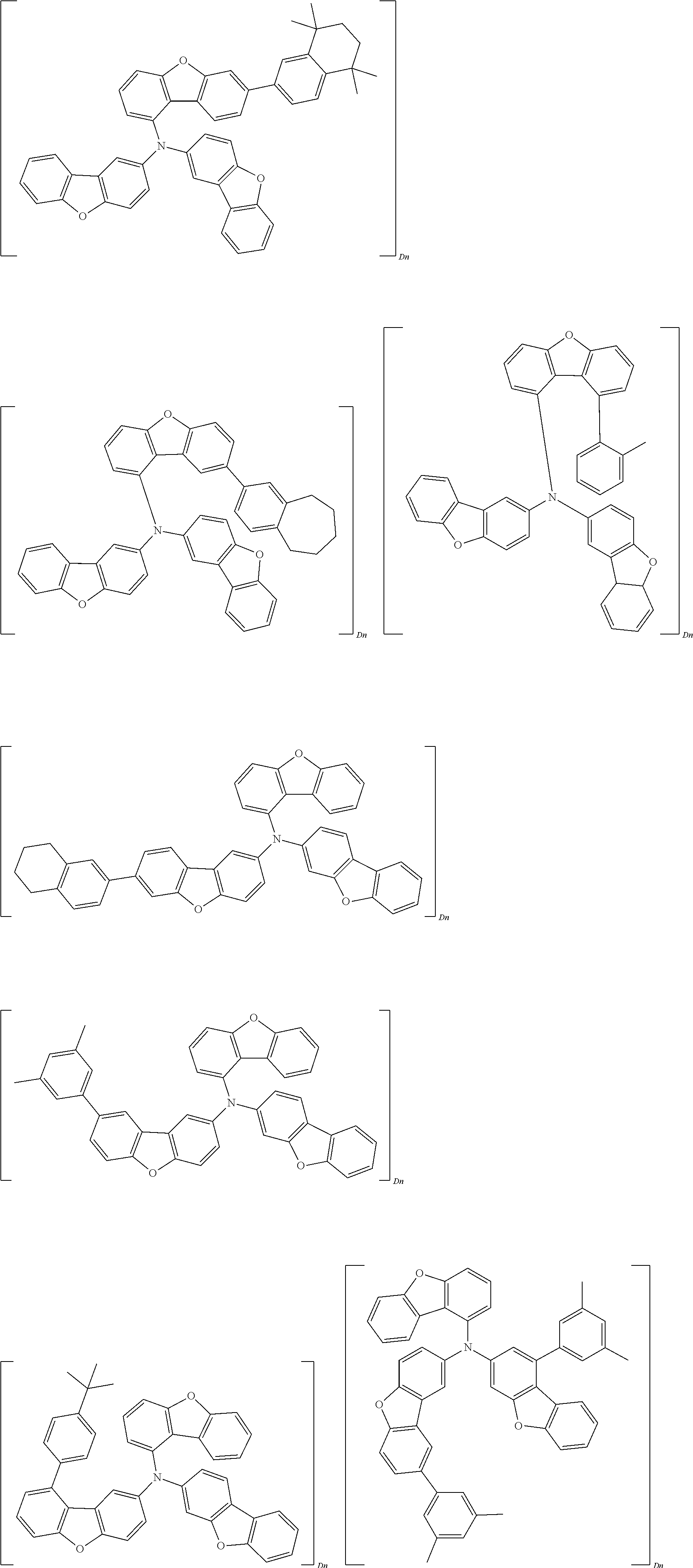

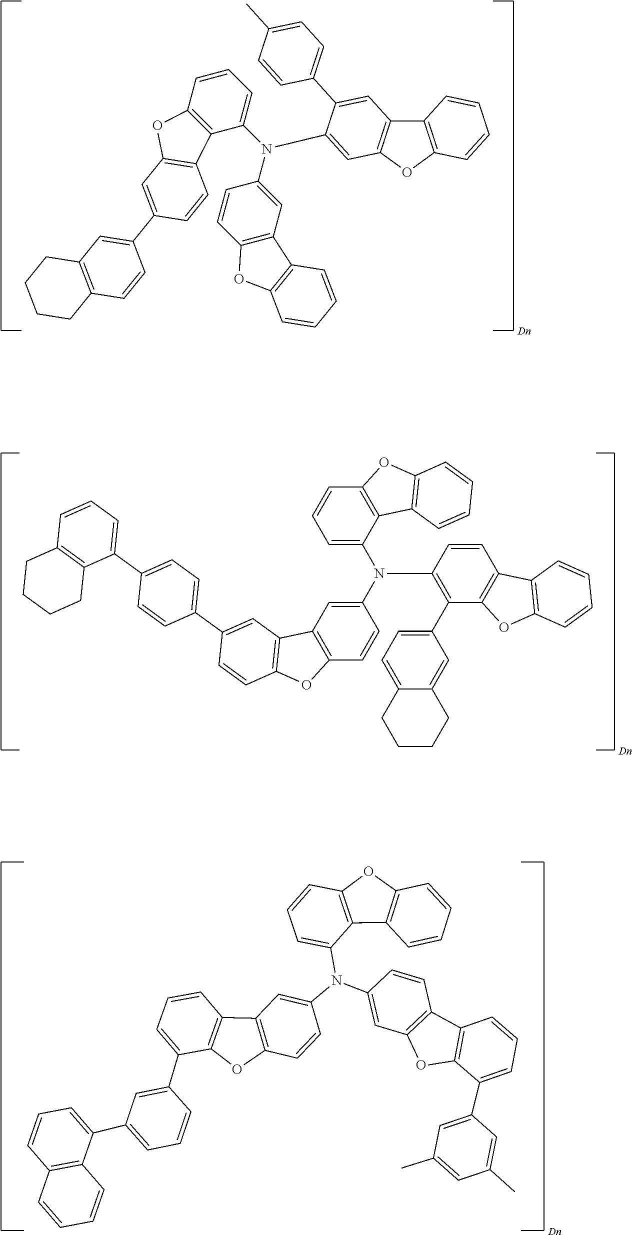

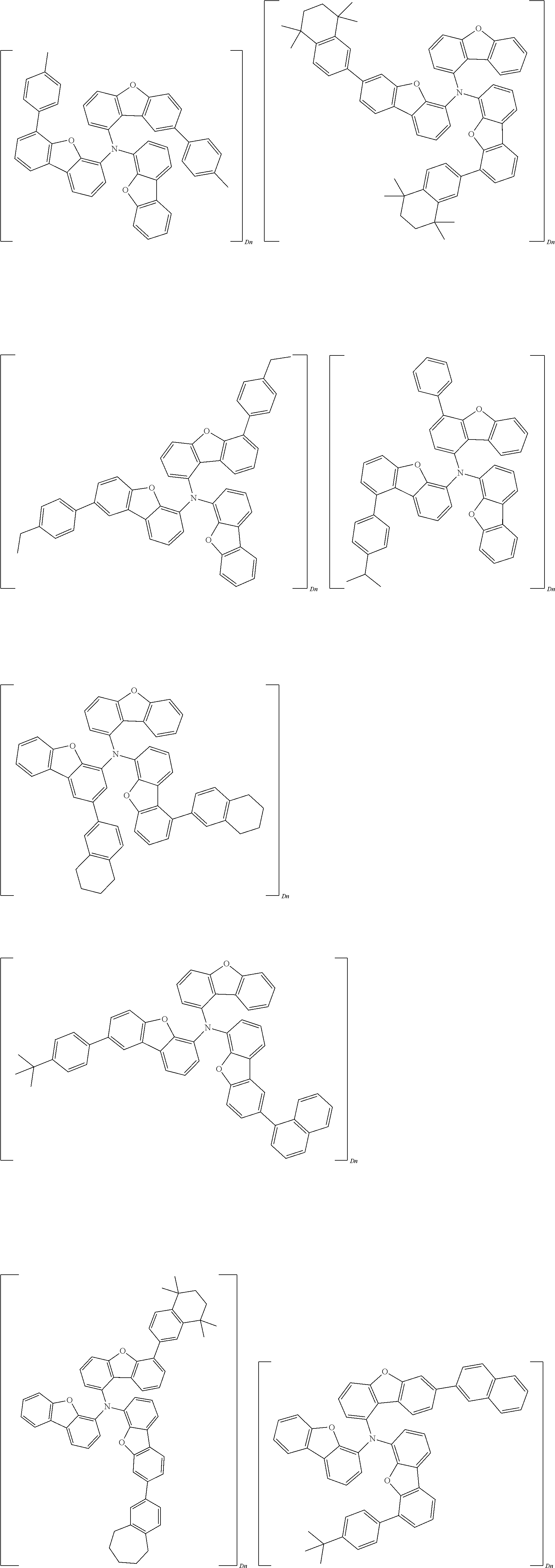

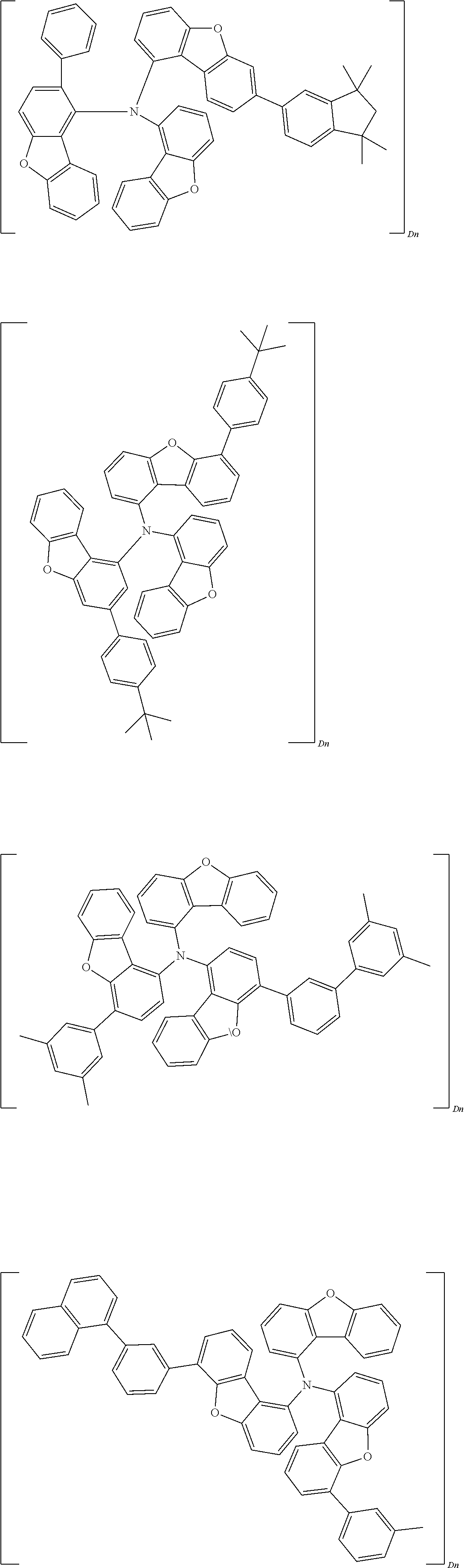

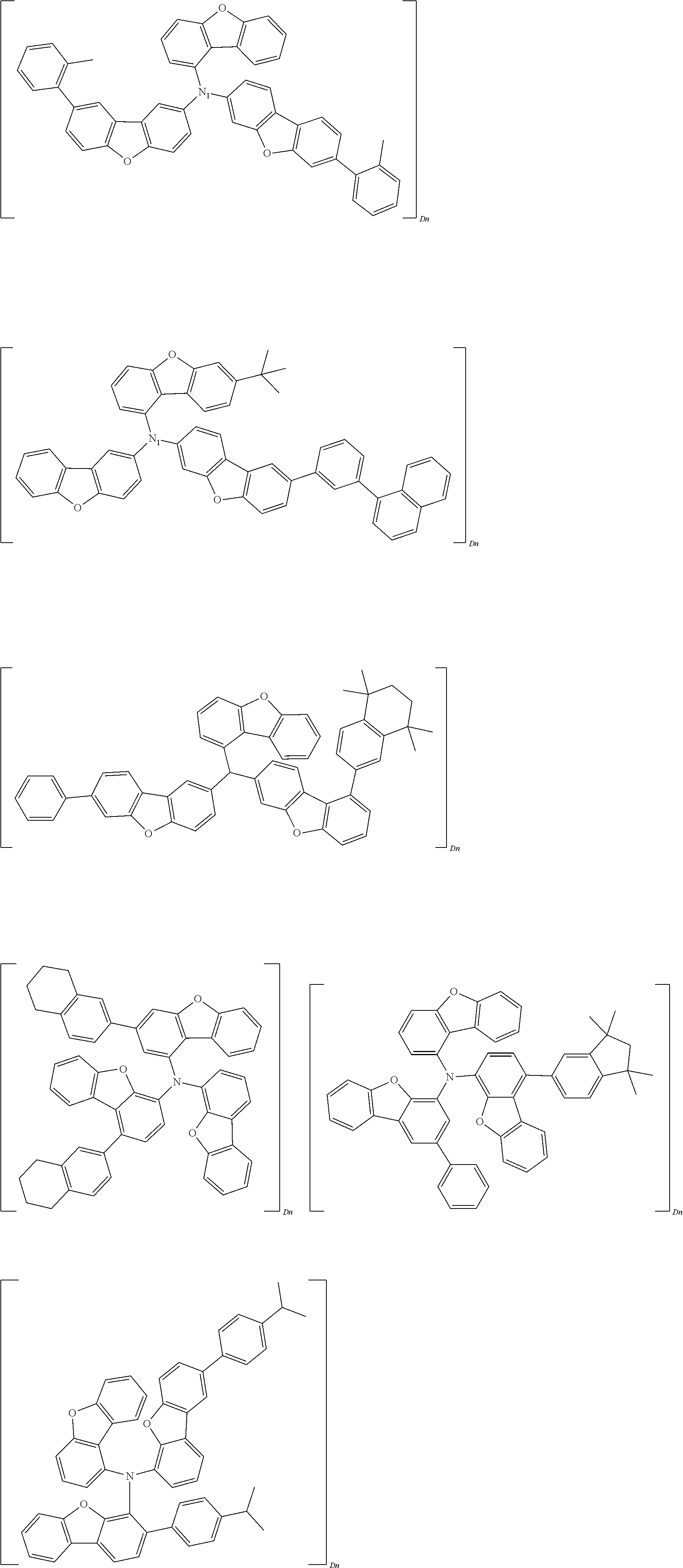

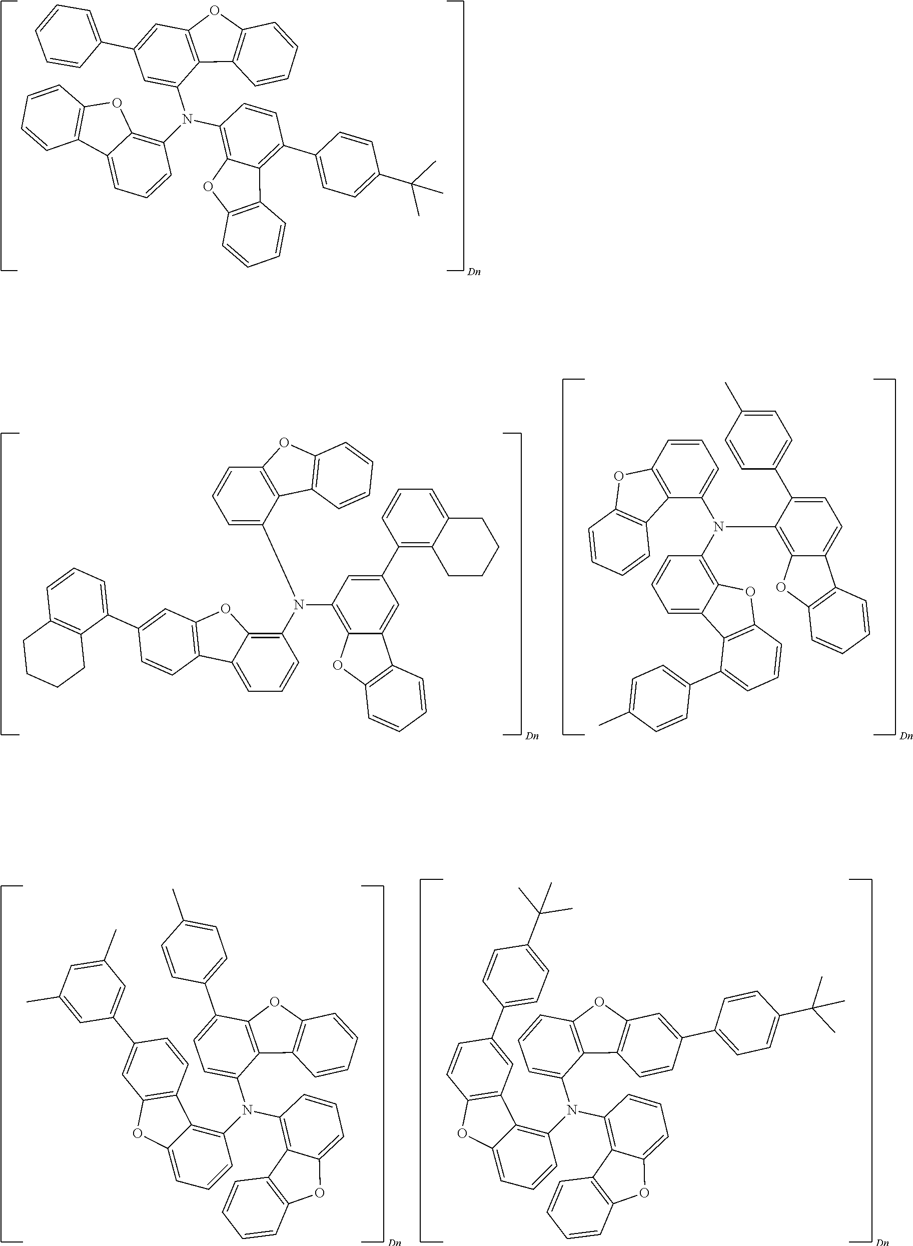

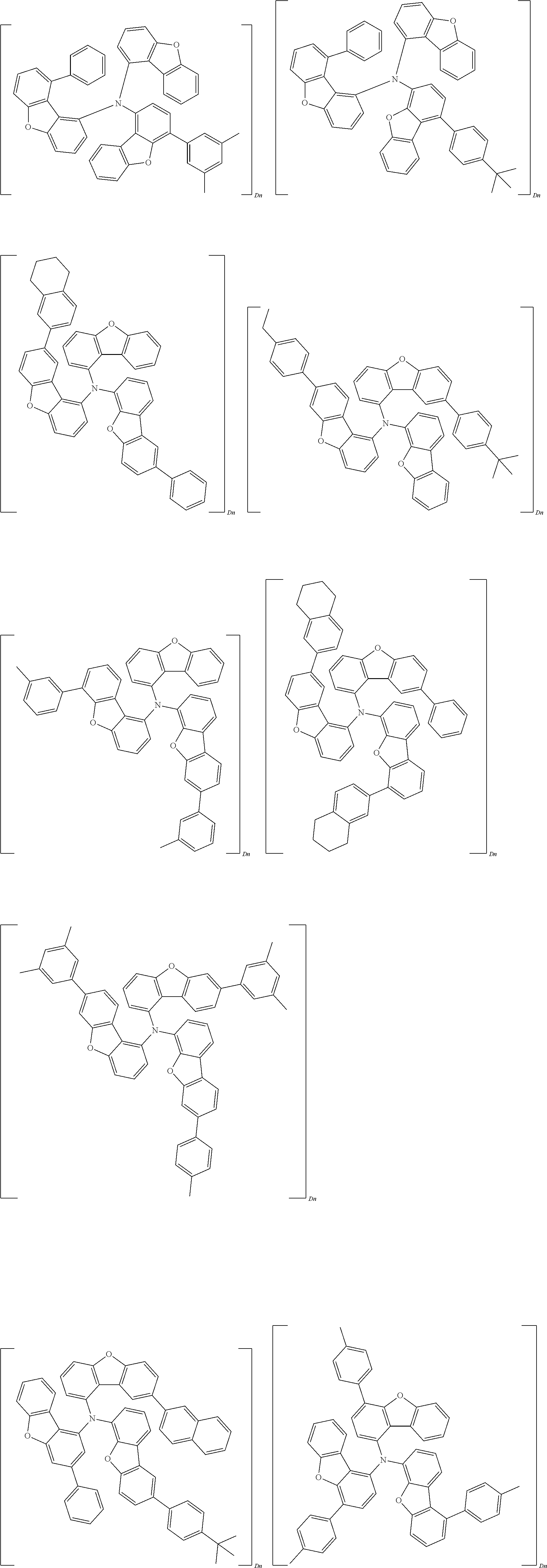

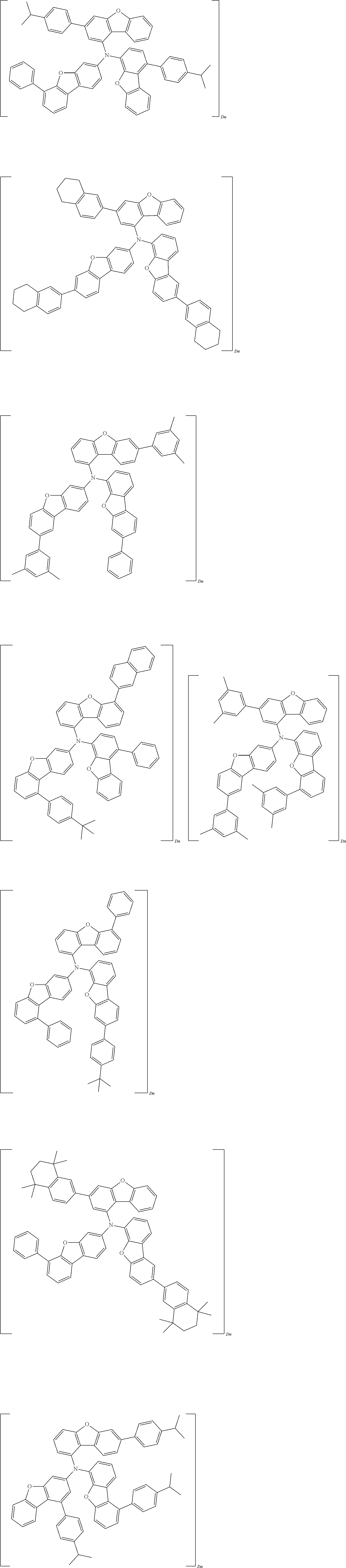

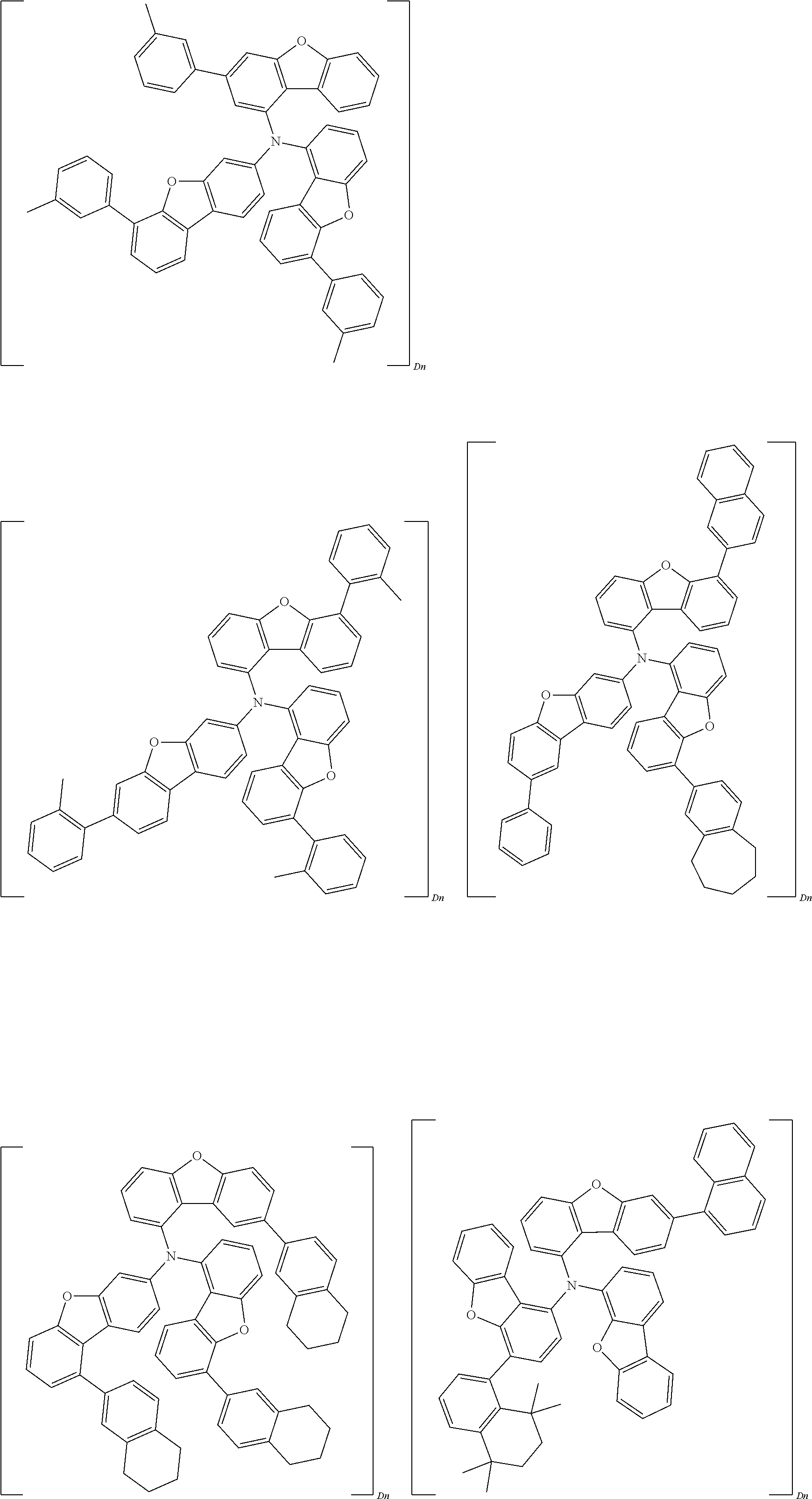

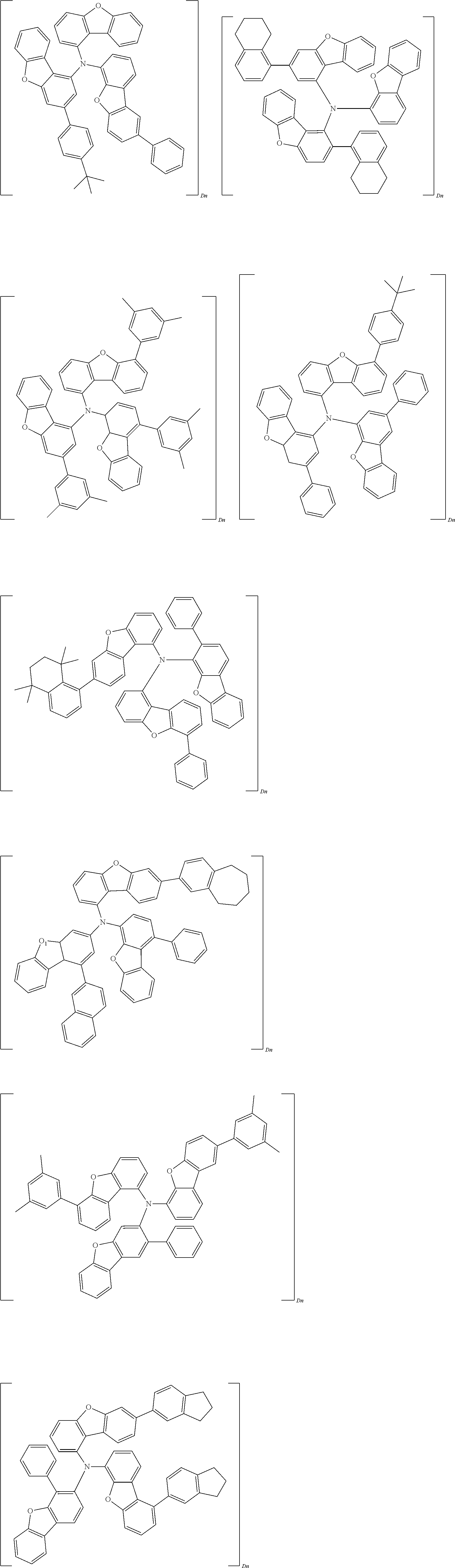

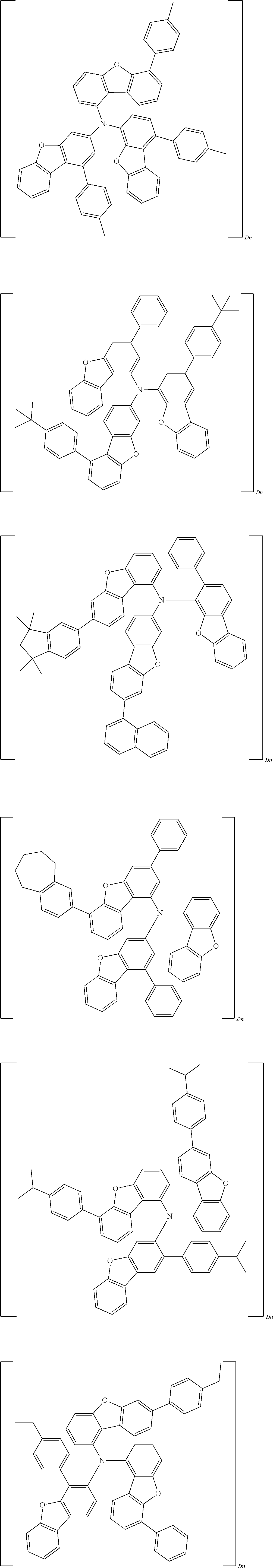

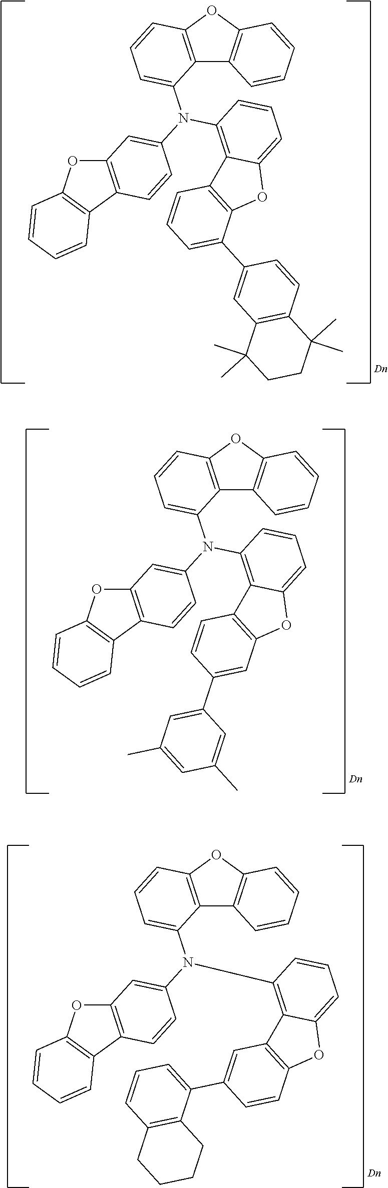

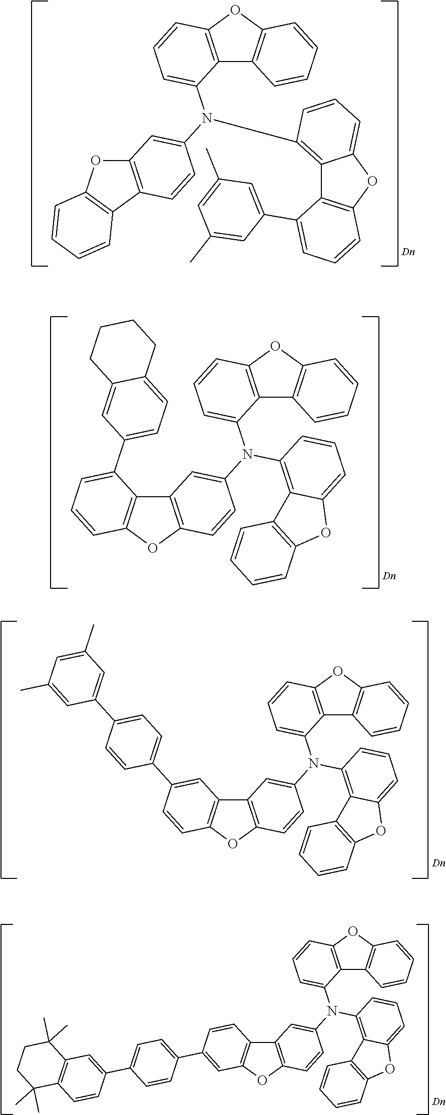

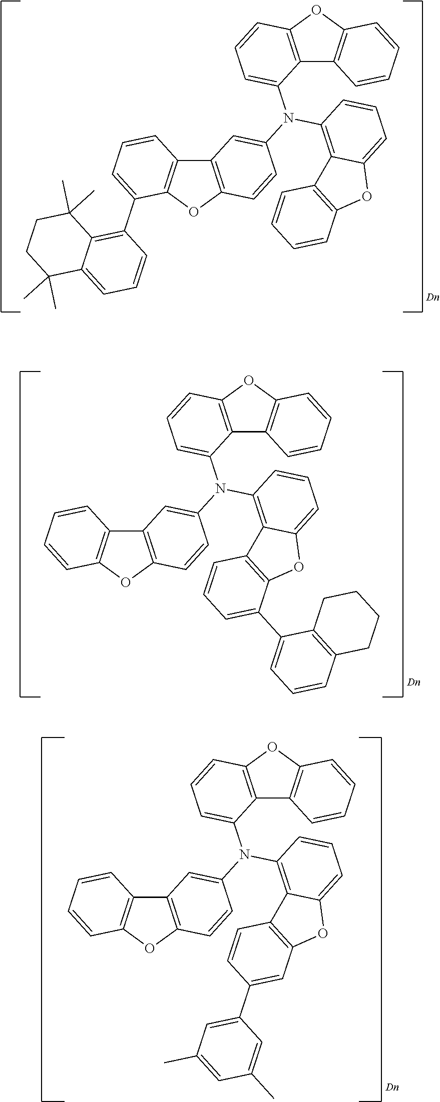

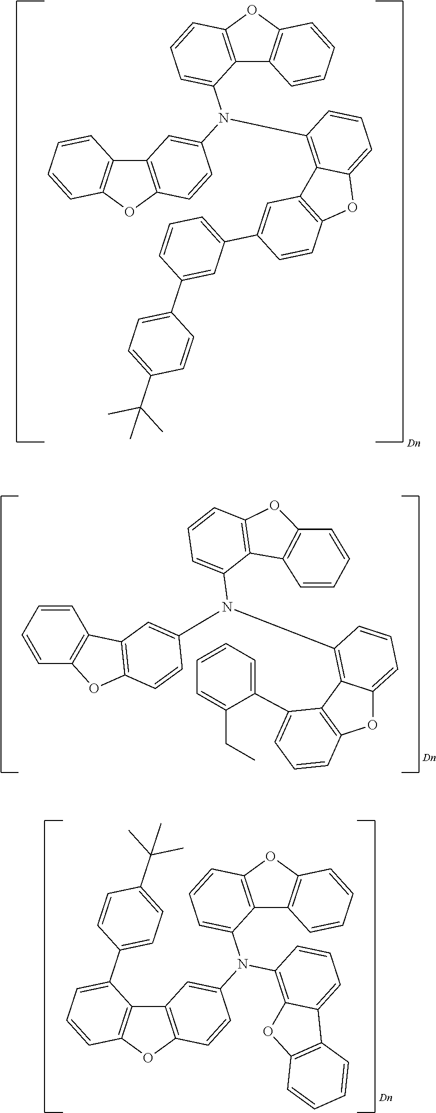

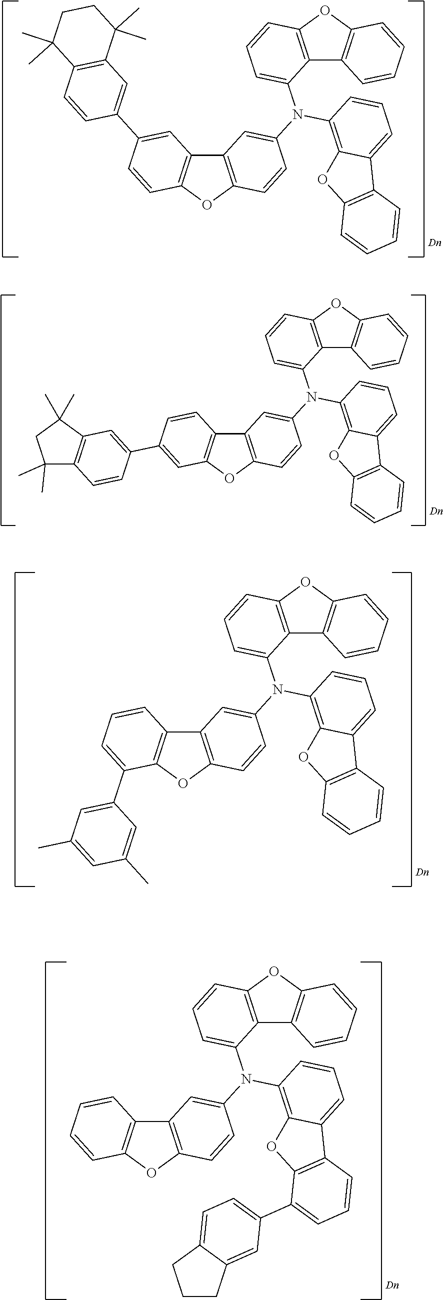

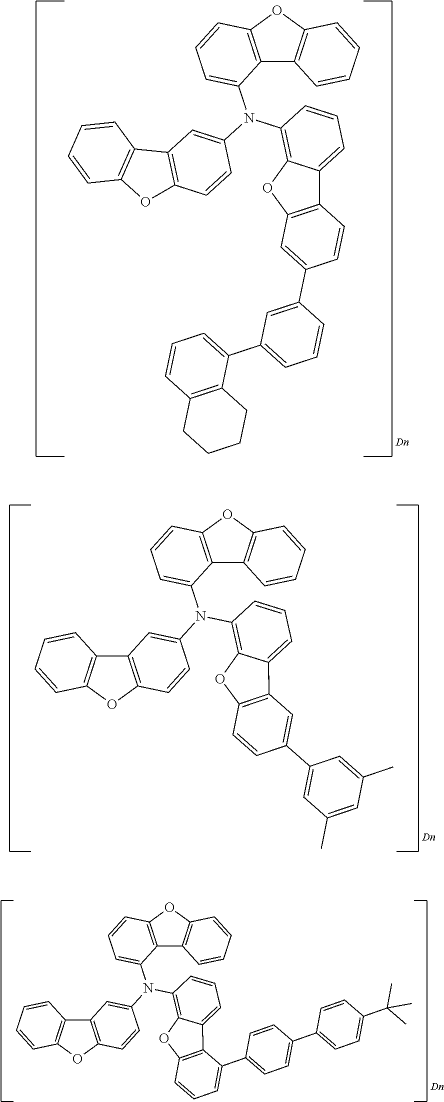

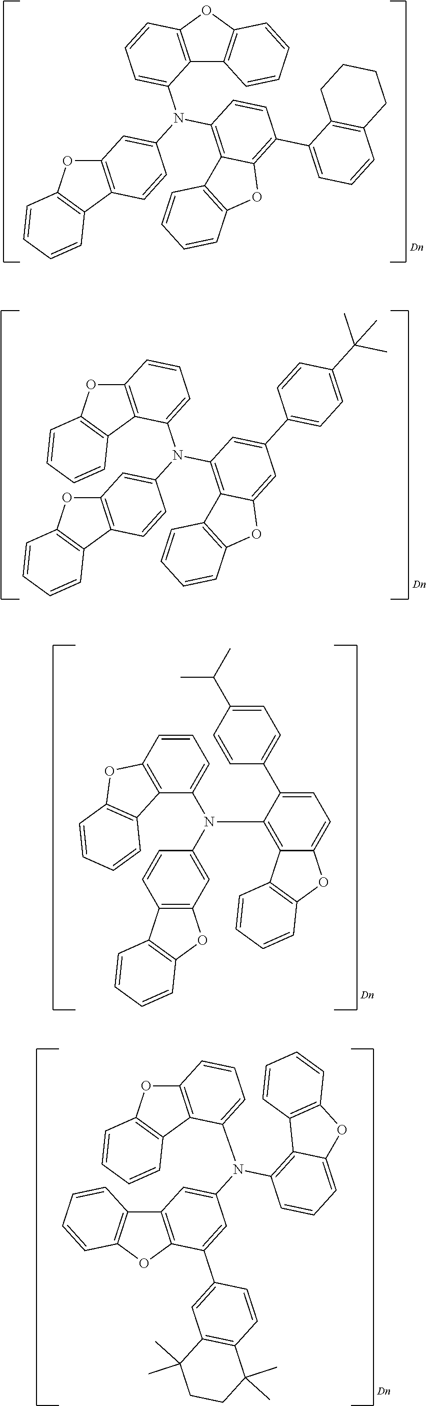

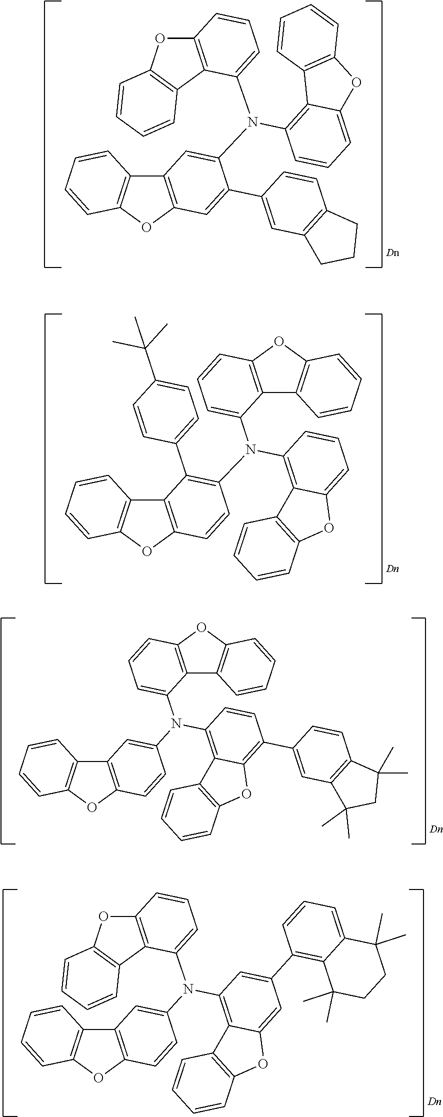

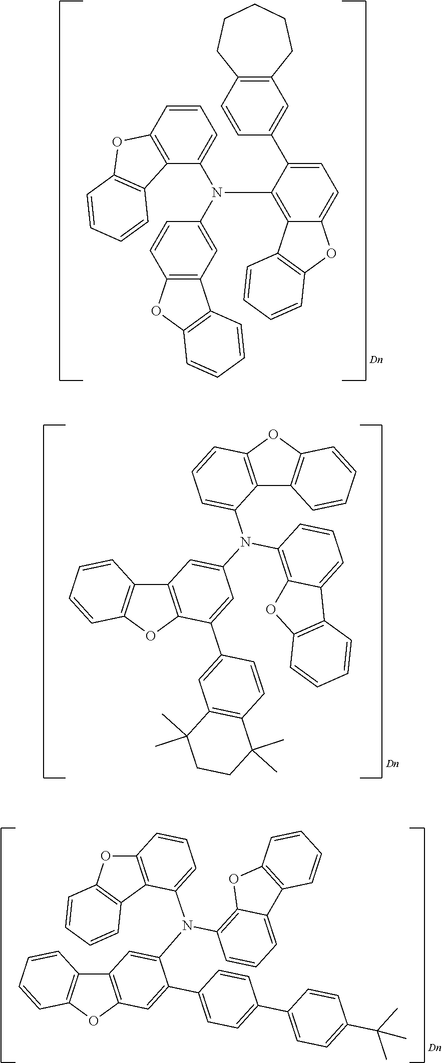

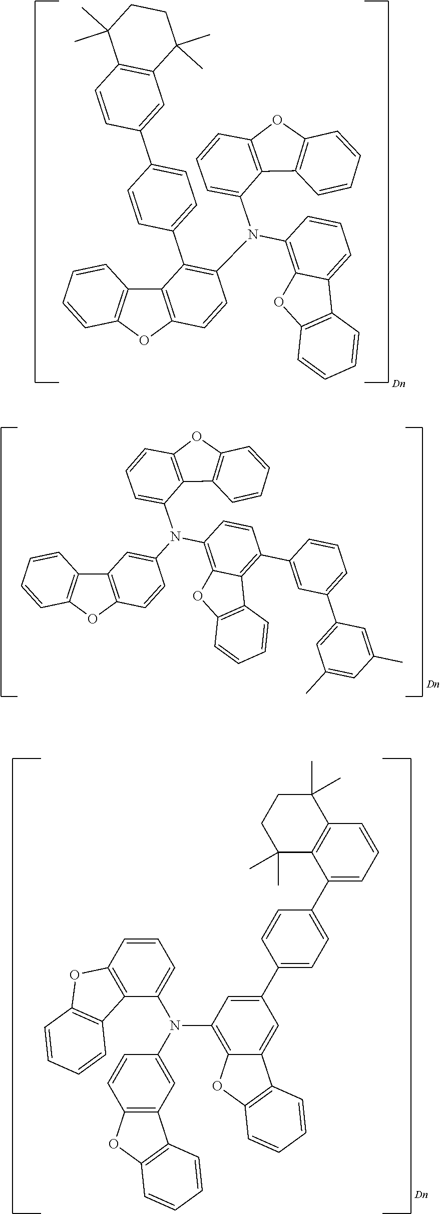

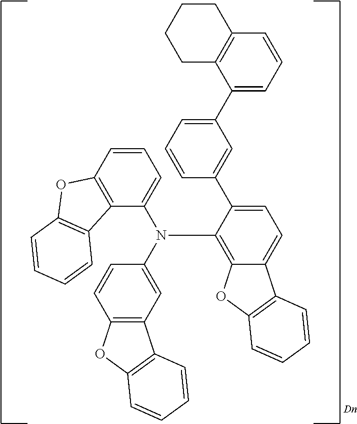

wherein the Dn means the number of deuterium (D) substitutions of each compound described in the square brackets, which is different for each compound, n is an integer of 0 or more, and the maximum value of n is the total number of hydrogens contained in each compound in the square brackets.

For example, since the total number of hydrogens in

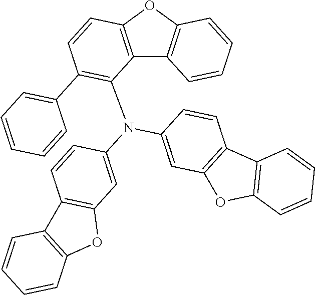 [SMILES: c1ccc(-c2ccc3oc4ccccc4c3c2N(c2ccc3c(c2)oc2ccccc23)c2ccc3c(c2)oc2ccccc23)cc1]

is 25, n in

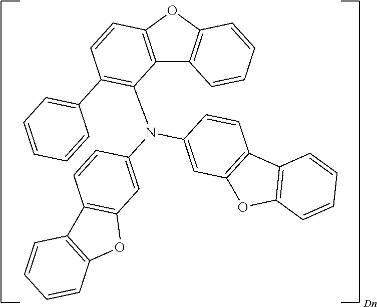

is an integer of 0 to 25,

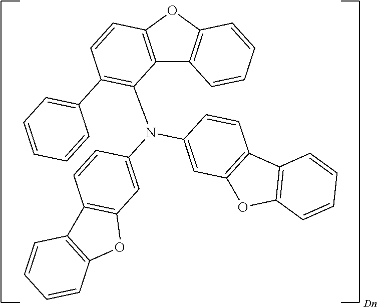

is used to encompass compounds in which 1 to 25 of the hydrogens

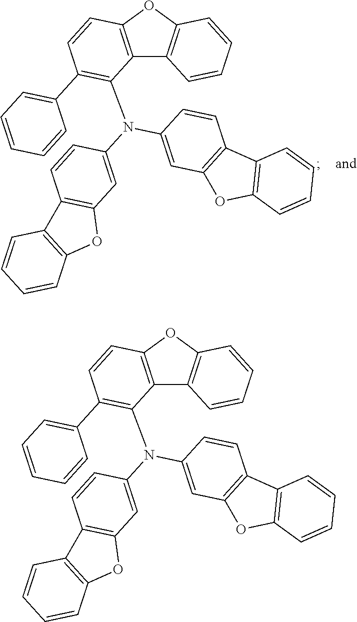 [SMILES: c1ccc(-c2ccc3oc4ccccc4c3c2N(c2ccc3c(c2)oc2ccccc23)c2ccc3c(c2)oc2ccccc23)cc1 ; c1ccc(-c2ccc3oc4ccccc4c3c2N(c2ccc3c(c2)oc2ccccc23)c2ccc3c(c2)oc2ccccc23)cc1]

which are not substituted with deuterium, are substituted with deuterium.

In addition, according to the present disclosure, there is provided a method for preparing the compound represented by Chemical Formula 1.

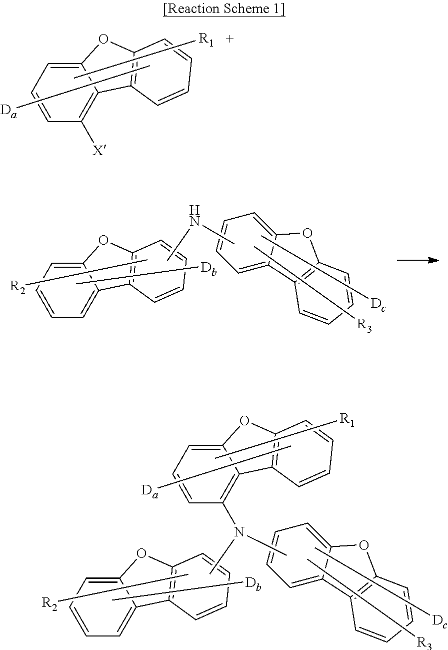

in Reaction Scheme 1, the remaining substituents except for X′ are the same as defined in Chemical Formula 1, and X′ is halogen. Preferably, X′ is chloro or bromo.

Reaction Scheme 1 is an amine substitution reaction, which is preferably carried out in the presence of a palladium catalyst and a base, and a reactive group for the amine substitution reaction can be changed as known in the art.

The method for preparing a compound represented by Chemical Formula 1 can be further embodied in Preparation Examples described hereinafter.

Further, according to the present disclosure, there is provided an organic light emitting device comprising a compound represented by Chemical Formula 1. In one example, the present disclosure provides an organic light emitting device comprising: a first electrode; a second electrode that is provided opposite to the first electrode; and one or more organic material layers that are provided between the first electrode and the second electrode, wherein at least one layer of the organic material layers includes the compound represented by Chemical Formula 1.

The organic material layer of the organic light emitting device of the present disclosure may have a single-layer structure, or it may have a multilayered structure in which two or more organic material layers are stacked. For example, the organic light emitting device of the present disclosure may have a structure comprising a hole injection layer, a hole transport layer, a hole transport auxiliary layer, a light emitting layer, an electron transport layer, an electron injection layer and the like as the organic material layer. However, the structure of the organic light emitting device is not limited thereto, and it may include a smaller number of organic layers.

Further, the organic material layer may include a hole injection layer, a hole transport layer, or a hole transport auxiliary layer, wherein the hole injection layer, the hole transport layer, or the hole transport auxiliary layer includes the compound represented by Chemical Formula 1.

Further, the organic light emitting device according to the present disclosure may be a normal type organic light emitting device in which an anode, one or more organic material layers, and a cathode are sequentially stacked on a substrate. Further, the organic light emitting device according to the present disclosure may be an inverted type organic light emitting device in which a cathode, one or more organic material layers, and an anode are sequentially stacked on a substrate. For example, the structure of the organic light emitting device according to an embodiment of the present disclosure is illustrated in FIGS. 1 to 3.

FIG. 1 shows an example of an organic light emitting device comprising a substrate 1, an anode 2, an organic material layer 3, and a cathode 4. In such a structure, the compound represented by Chemical Formula 1 may be included in the organic material layer.

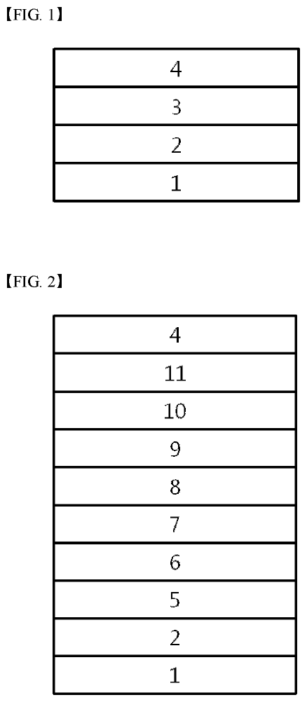

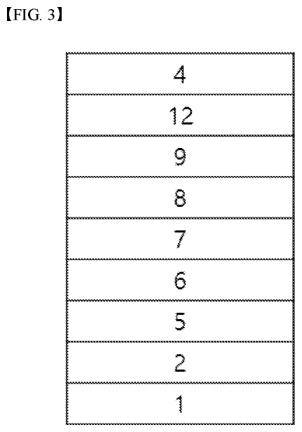

FIG. 2 shows an example of an organic light emitting device comprising a substrate 1, an anode 2, a hole injection layer 5, a hole transport layer 6, a hole transport auxiliary layer 7, a light emitting layer 8, a hole blocking layer 9, an electron transport layer 10, an electron injection layer 11, and a cathode 4. In such a structure, the compound represented by Chemical Formula 1 may be included in the hole transport auxiliary layer by way of example.

FIG. 3 shows an example of an organic light emitting device comprising a substrate 1, an anode 2, a hole injection layer 5, a hole transport layer 6, a hole transport auxiliary layer 7, a light emitting layer 8, a hole blocking layer 9, an electron injection and transport layer 12, and cathode 4. In such a structure, the compound represented by Chemical Formula 1 may be included in the hole transport auxiliary layer by way of example.

The organic light emitting device according to the present disclosure may be manufactured by materials and methods known in the art, except that at least one of the organic material layers includes the compound represented by Chemical Formula 1. Further, when the organic light emitting device includes a plurality of organic material layers, the organic material layers may be formed of the same material or different materials.

For example, the organic light emitting device according to the present disclosure can be manufactured by sequentially stacking a first electrode, an organic material layer and a second electrode on a substrate. In this case, the organic light emitting device may be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the hole transport auxiliary layer, the light emitting layer, the hole blocking layer, the electron injection and transport layer, and the like thereon, and then depositing a material that can be used as the cathode thereon. In addition to such a method, the organic light emitting device can be manufactured by sequentially depositing a cathode material, an organic material layer and an anode material on a substrate.

Further, the compound represented by Chemical Formula 1 can be formed into an organic layer by a solution coating method as well as a vacuum deposition method at the time of manufacturing an organic light emitting device. Wherein, the solution coating method means a spin coating, a dip coating, a doctor blading, an inkjet printing, a screen printing, a spray method, a roll coating, or the like, but is not limited thereto.

In addition to such a method, the organic light emitting device may be manufactured by sequentially depositing a cathode material, an organic material layer and an anode material on a substrate (International Publication WO2003/012890). However, the manufacturing method is not limited thereto.

In one example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to a hole injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrin, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes to the light emitting layer. The hole transport material is suitably a material having large mobility to the holes, which may receive holes from the anode or the hole injection layer and transfer the holes to the light emitting layer. Specific examples thereof include an arylamine-based organic material, a conductive polymer, a block copolymer in which a conjugate portion and a non-conjugate portion are present together, and the like, but are not limited thereto.

The hole transport auxiliary layer is a layer located between the hole transport layer and the light emitting layer to adjust the transfer speed or injection speed of holes, and is not particularly limited as long as it is a material with a low hole injection barrier and high hole mobility, and any material known in the art can be used without limitation. In one embodiment, the compound represented by Chemical Formula 1 can be used as the hole transport auxiliary layer material, and in this case, it can smoothly transfer holes to the light emitting layer and increase the stability of excitons formed in the light emitting layer, which is preferable.

An electron blocking layer may be interposed between the hole transport auxiliary layer and the light emitting layer. The electron blocking layer serves to inhibit the electrons injected in the cathode from being transferred to the anode side without being recombined in the light emitting layer, thereby improving the efficiency of the organic light emitting device. The electron blocking layer is preferably a material having the smaller electron affinity than the electron transport layer.

The light emitting material is preferably a material which may receive holes and electrons transported from a hole transport layer and an electron transport layer, respectively, and combine the holes and the electrons to emit light in a visible ray region, and has good quantum efficiency to fluorescence or phosphorescence. Specific examples of the light emitting material include an 8-hydroxy-quinoline aluminum complex (Alq₃); a carbazole-based compound; a dimerized styryl compound; BAlq; a 10-hydroxybenzoquinoline-metal compound; a benzoxazole, benzthiazole and benzimidazole-based compound; a poly(p-phenylenevinylene)(PPV)-based polymer; a spiro compound; polyfluorene, lubrene, and the like, but are not limited thereto.

The light emitting layer may include a host material and a dopant material. The host material includes a fused aromatic ring derivative, a heterocycle-containing compound, or the like. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto. Particularly, in the present disclosure, the compound represented by Chemical Formula 1 can be used as a host material of the light emitting layer, and in this case, low voltage, high efficiency, and/or long lifetime characteristics of the organic light emitting device can be obtained.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

The electron transport layer is a layer that may receive the electrons from the electron injection layer and transport the electrons to the light emitting layer, and an electron transport material is suitably a material which may receive well injection of electrons from a cathode and transfer the electrons to a light emitting layer, and has a large mobility for electrons. Specific examples of the electron transport material include: an Al complex of 8-hydroxyquinoline; a complex including Alq₃; an organic radical compound; a hydroxyflavone-metal complex, and the like, but are not limited thereto. The electron transport layer may be used with any desired cathode material, as used according to a conventional technique. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples of the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

The organic light emitting device according to the present disclosure may, instead of an electron transport layer and an electron injection layer, include an electron injection and transport layer that simultaneously performs the roles of these layers.

The organic light emitting device according to the present disclosure may be a bottom emission type device, a top emission type device, or a double side emission type device, and in particular, it may be a bottom emission type light emitting device that requires relatively high luminous efficiency.

In addition, the compound represented by Chemical Formula 1 may be included in an organic solar cell or an organic transistor in addition to an organic light emitting device.

The preparation of the compound represented by Chemical Formula 1 and the organic light emitting device including the same will be described in detail in the following examples. However, these examples are presented for illustrative purposes only, and are not intended to limit the scope of the present disclosure.

### SYNTHESIS EXAMPLE

### Synthesis Example 1

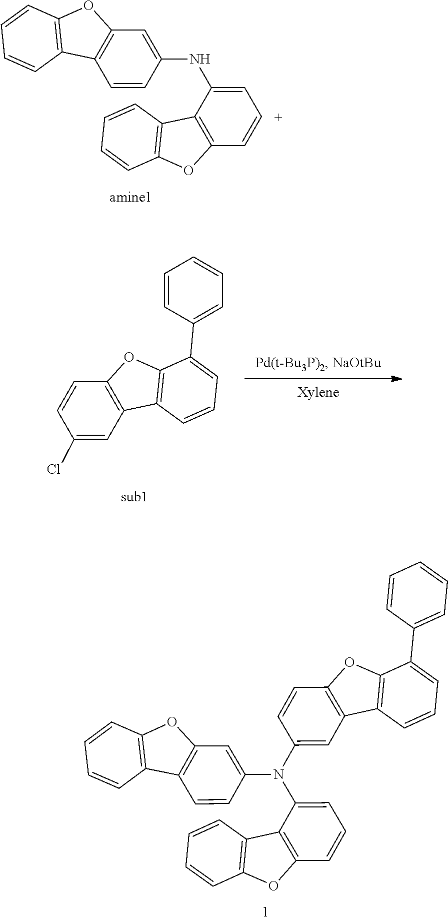 [SMILES: c1ccc2c(c1)oc1cc(Nc3cccc4oc5ccccc5c34)ccc12 ; Clc1ccc2oc3c(-c4ccccc4)cccc3c2c1 ; c1ccc(-c2cccc3c2oc2ccc(N(c4ccc5c(c4)oc4ccccc45)c4cccc5oc6ccccc6c45)cc23)cc1]

amine1 (15 g, 42.9 mmol), sub1 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15.7 g of Compound 1. (Yield: 62%, MS: [M+H]+=592)

### Synthesis Example 2

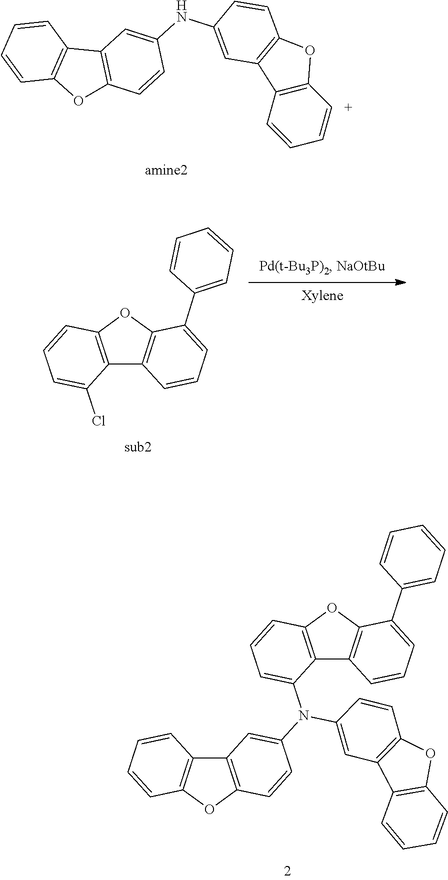 [SMILES: c1ccc2c(c1)oc1ccc(Nc3ccc4oc5ccccc5c4c3)cc12 ; Clc1cccc2oc3c(-c4ccccc4)cccc3c12 ; c1ccc(-c2cccc3c2oc2cccc(N(c4ccc5oc6ccccc6c5c4)c4ccc5oc6ccccc6c5c4)c23)cc1]

amine2 (15 g, 42.9 mmol), sub2 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 13.2 g of Compound 2. (Yield: 52%, MS: [M+H]+=592)

### Synthesis Example 3

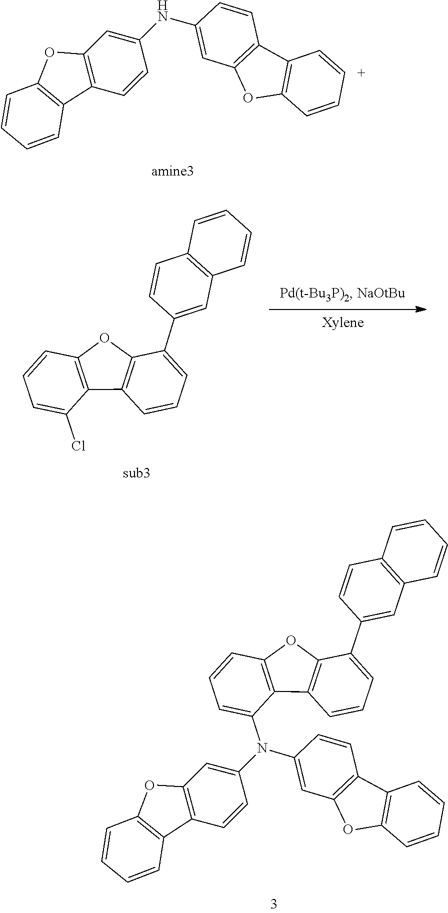 [SMILES: c1ccc2c(c1)oc1cc(Nc3ccc4c(c3)oc3ccccc34)ccc12 ; Clc1cccc2oc3c(-c4ccc5ccccc5c4)cccc3c12 ; c1ccc2cc(-c3cccc4c3oc3cccc(N(c5ccc6c(c5)oc5ccccc56)c5ccc6c(c5)oc5ccccc56)c34)ccc2c1]

amine3 (15 g, 42.9 mmol), sub3 (14.8 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.6 g of Compound 3. (Yield: 53%, MS: [M+H]+=642)

### Synthesis Example 4

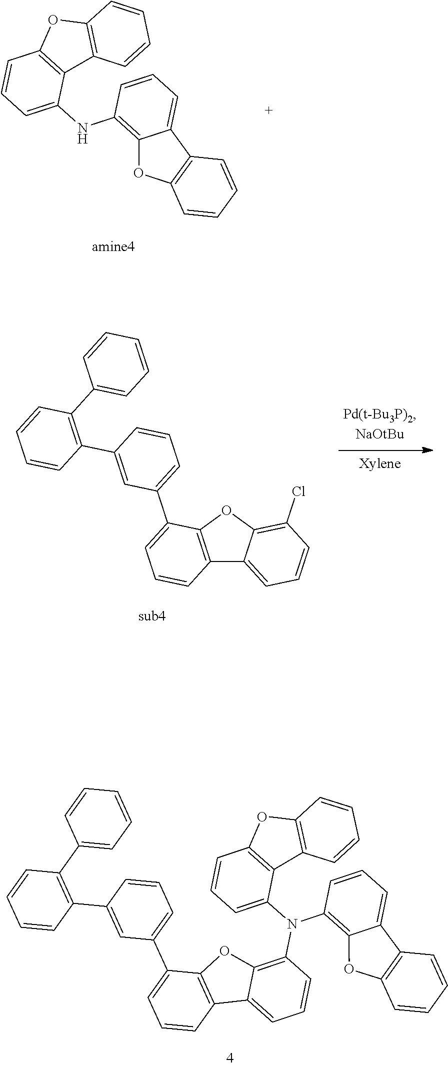 [SMILES: c1ccc2c(c1)oc1c(Nc3cccc4oc5ccccc5c34)cccc12 ; Clc1cccc2c1oc1c(-c3cccc(-c4ccccc4-c4ccccc4)c3)cccc12 ; c1ccc(-c2ccccc2-c2cccc(-c3cccc4c3oc3c(N(c5cccc6c5oc5ccccc56)c5cccc6oc7ccccc7c56)cccc34)c2)cc1]

amine4 (15 g, 42.9 mmol), sub4 (19.4 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 19.1 g of Compound 4. (Yield: 60%, MS: [M+H]+=744)

### Synthesis Example 5

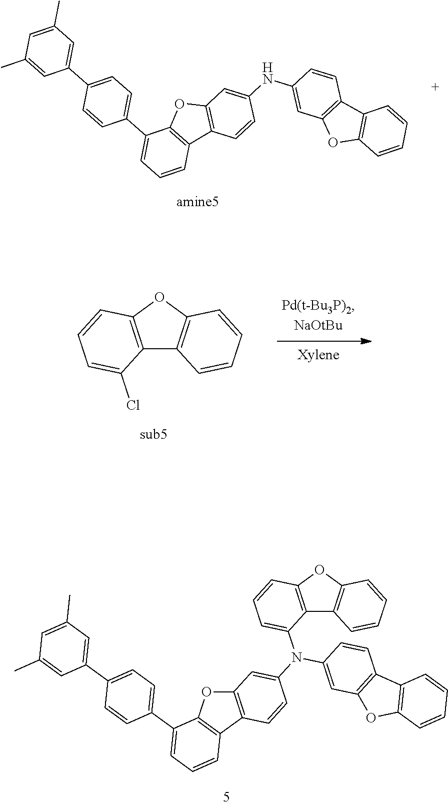 [SMILES: Cc1cc(C)cc(-c2ccc(-c3cccc4c3oc3cc(Nc5ccc6c(c5)oc5ccccc56)ccc34)cc2)c1 ; Clc1cccc2oc3ccccc3c12 ; Cc1cc(C)cc(-c2ccc(-c3cccc4c3oc3cc(N(c5ccc6c(c5)oc5ccccc56)c5cccc6oc7ccccc7c56)ccc34)cc2)c1]

amine5 (15 g, 28.3 mmol), sub5 (6 g, 29.7 mmol) and sodium tert-butoxide (4.1 g, 42.5 mmol) were added to 300 ml of xylene under a nitrogen atmosphere, and mixture was stirred and refluxed. Then, bis(tri-tert-butylphosphine)palladium(0) (0.1 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 11.2 g of Compound 5. (Yield: 57%, MS: [M+H]+=696)

### Synthesis Example 6

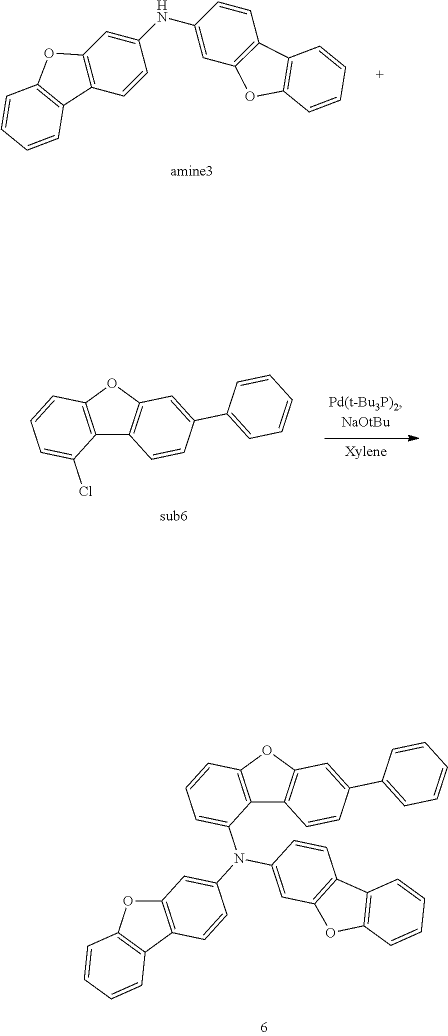 [SMILES: c1ccc2c(c1)oc1cc(Nc3ccc4c(c3)oc3ccccc34)ccc12 ; Clc1cccc2oc3cc(-c4ccccc4)ccc3c12 ; c1ccc(-c2ccc3c(c2)oc2cccc(N(c4ccc5c(c4)oc4ccccc45)c4ccc5c(c4)oc4ccccc45)c23)cc1]

amine3 (15 g, 42.9 mmol), sub6 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 17.5 g of Compound 6. (Yield: 69%, MS: [M+H]+=592)

### Synthesis Example 7

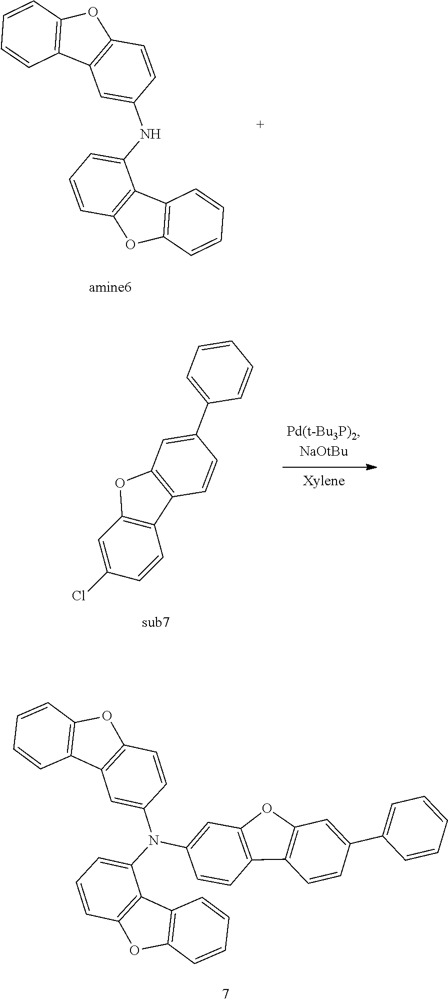 [SMILES: c1ccc2c(c1)oc1ccc(Nc3cccc4oc5ccccc5c34)cc12 ; Clc1ccc2c(c1)oc1cc(-c3ccccc3)ccc12 ; c1ccc(-c2ccc3c(c2)oc2cc(N(c4ccc5oc6ccccc6c5c4)c4cccc5oc6ccccc6c45)ccc23)cc1]

amine6 (15 g, 42.9 mmol), sub7 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15.5 g of Compound 7. (Yield: 61%, MS: [M+H]+=592)

### Synthesis Example 8

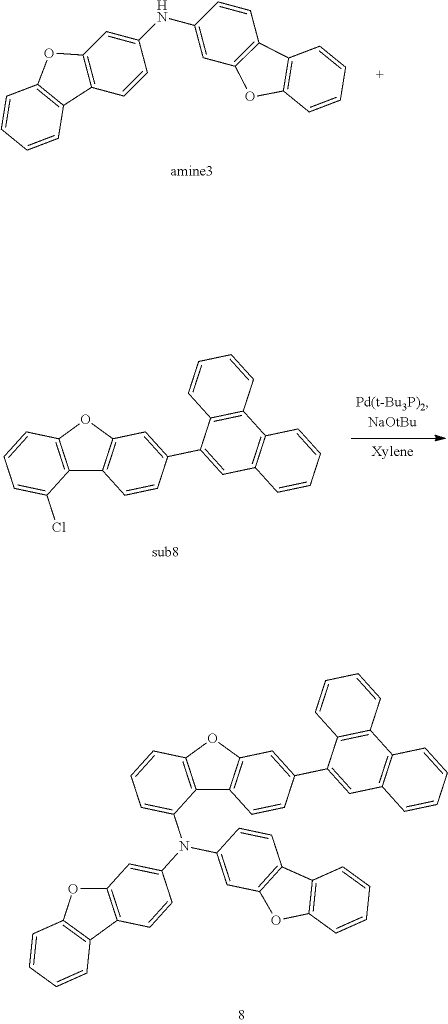 [SMILES: c1ccc2c(c1)oc1cc(Nc3ccc4c(c3)oc3ccccc34)ccc12 ; Clc1cccc2oc3cc(-c4cc5ccccc5c5ccccc45)ccc3c12 ; c1ccc2c(c1)cc(-c1ccc3c(c1)oc1cccc(N(c4ccc5c(c4)oc4ccccc45)c4ccc5c(c4)oc4ccccc45)c13)c1ccccc12]

amine3 (15 g, 42.9 mmol), sub8 (17.1 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 20.2 g of Compound 8. (Yield: 68%, MS: [M+H]+=692)

### Synthesis Example 9

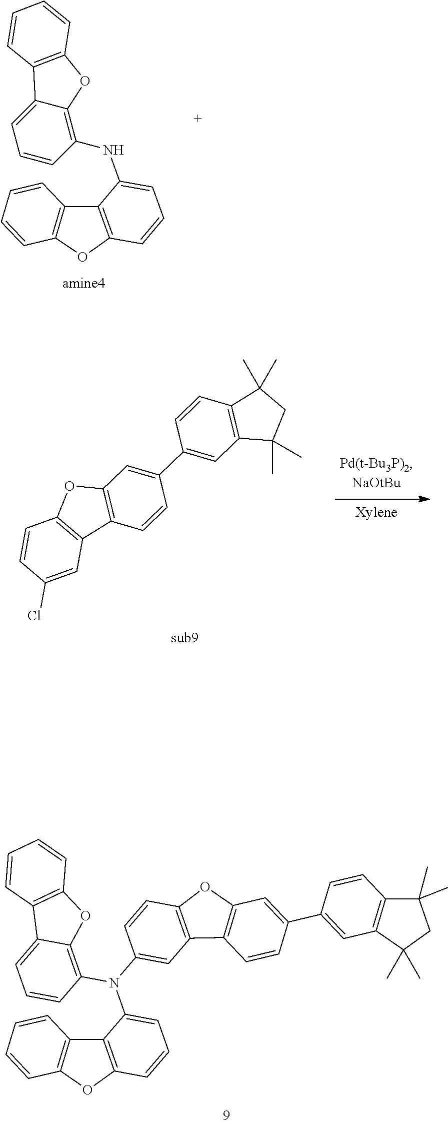 [SMILES: c1ccc2c(c1)oc1c(Nc3cccc4oc5ccccc5c34)cccc12 ; CC1(C)CC(C)(C)c2cc(-c3ccc4c(c3)oc3ccc(Cl)cc34)ccc21 ; CC1(C)CC(C)(C)c2cc(-c3ccc4c(c3)oc3ccc(N(c5cccc6c5oc5ccccc56)c5cccc6oc7ccccc7c56)cc34)ccc21]

amine4 (15 g, 42.9 mmol), sub9 (16.9 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 16.2 g of Compound 9. (Yield: 55%, MS: [M+H]+=688)

### Synthesis Example 10

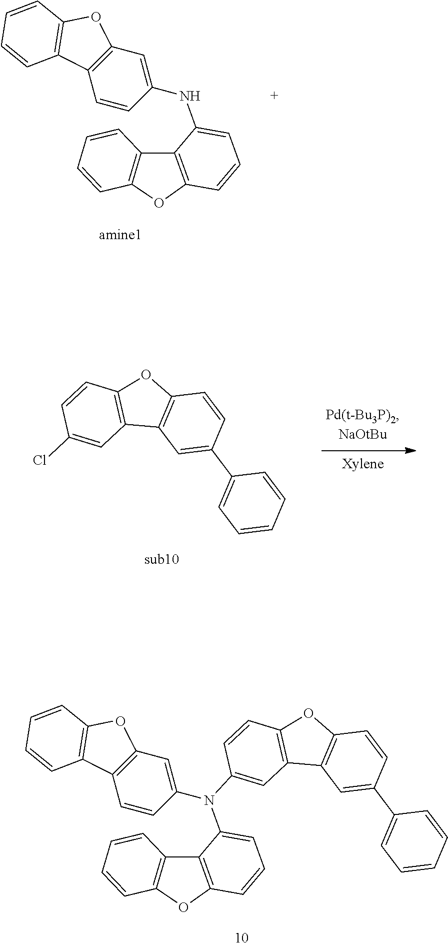 [SMILES: c1ccc2c(c1)oc1cc(Nc3cccc4oc5ccccc5c34)ccc12 ; Clc1ccc2oc3ccc(-c4ccccc4)cc3c2c1 ; c1ccc(-c2ccc3oc4ccc(N(c5ccc6c(c5)oc5ccccc56)c5cccc6oc7ccccc7c56)cc4c3c2)cc1]

amine1 (15 g, 42.9 mmol), sub10 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.7 g of Compound 10. (Yield: 58%, MS: [M+H]+=592)

### Synthesis Example 11

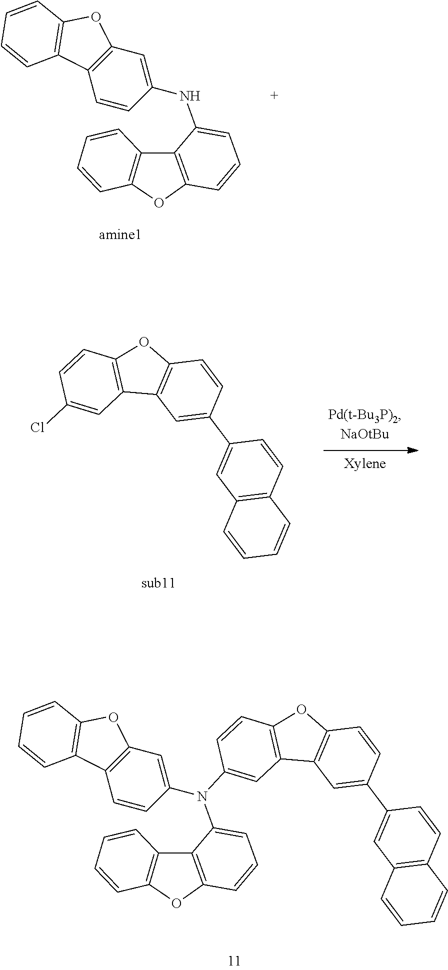 [SMILES: c1ccc2c(c1)oc1cc(Nc3cccc4oc5ccccc5c34)ccc12 ; Clc1ccc2oc3ccc(-c4ccc5ccccc5c4)cc3c2c1 ; c1ccc2cc(-c3ccc4oc5ccc(N(c6ccc7c(c6)oc6ccccc67)c6cccc7oc8ccccc8c67)cc5c4c3)ccc2c1]

amine1 (15 g, 42.9 mmol), sub11 (14.8 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 19 g of Compound 11. (Yield: 69%, MS: [M+H]+=642)

### Synthesis Example 12

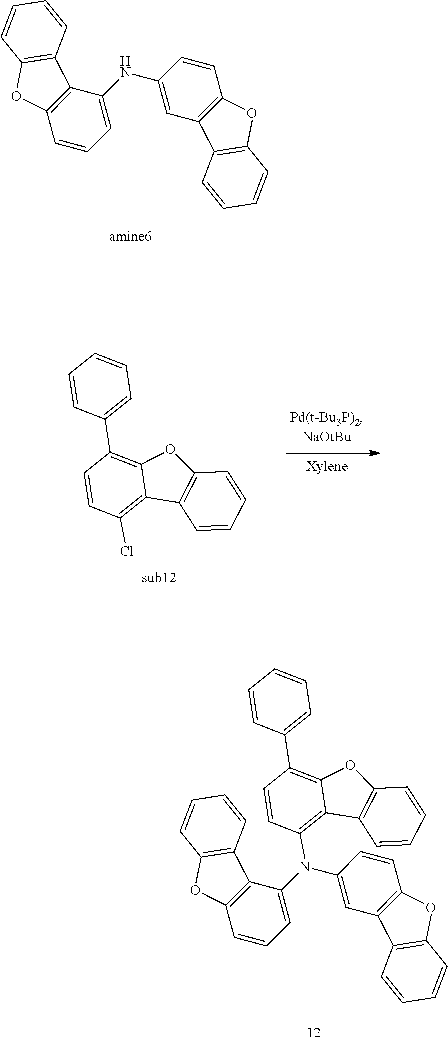 [SMILES: c1ccc2c(c1)oc1ccc(Nc3cccc4oc5ccccc5c34)cc12 ; Clc1ccc(-c2ccccc2)c2oc3ccccc3c12 ; c1ccc(-c2ccc(N(c3ccc4oc5ccccc5c4c3)c3cccc4oc5ccccc5c34)c3c2oc2ccccc23)cc1]

amine6 (15 g, 42.9 mmol), sub12 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15 g of Compound 12. (Yield: 59%, MS: [M+H]+=592)

### Synthesis Example 13

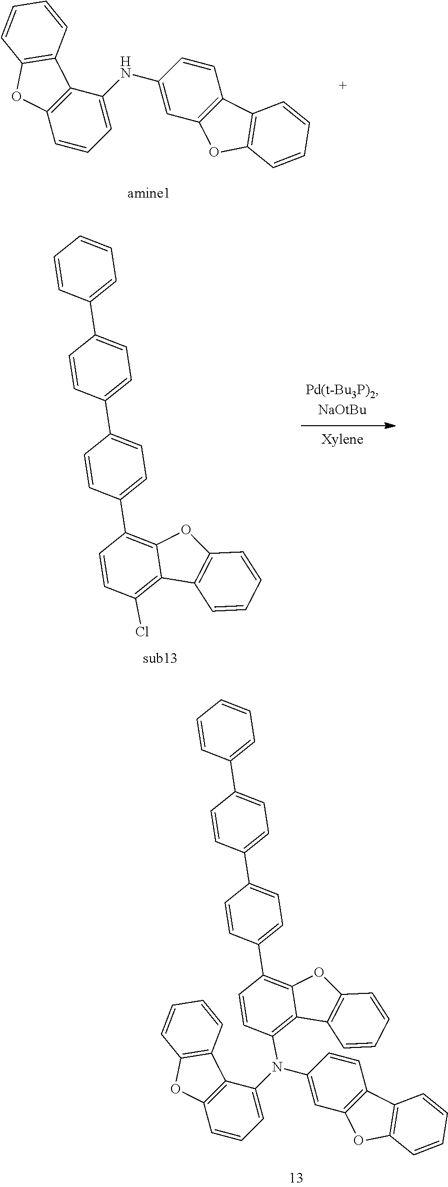 [SMILES: c1ccc2c(c1)oc1cc(Nc3cccc4oc5ccccc5c34)ccc12 ; Clc1ccc(-c2ccc(-c3ccc(-c4ccccc4)cc3)cc2)c2oc3ccccc3c12 ; c1ccc(-c2ccc(-c3ccc(-c4ccc(N(c5ccc6c(c5)oc5ccccc56)c5cccc6oc7ccccc7c56)c5c4oc4ccccc45)cc3)cc2)cc1]

amine1 (15 g, 42.9 mmol), sub13 (19.4 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 19.5 g of Compound 13. (Yield: 61%, MS: [M+H]+=744)

### Synthesis Example 14

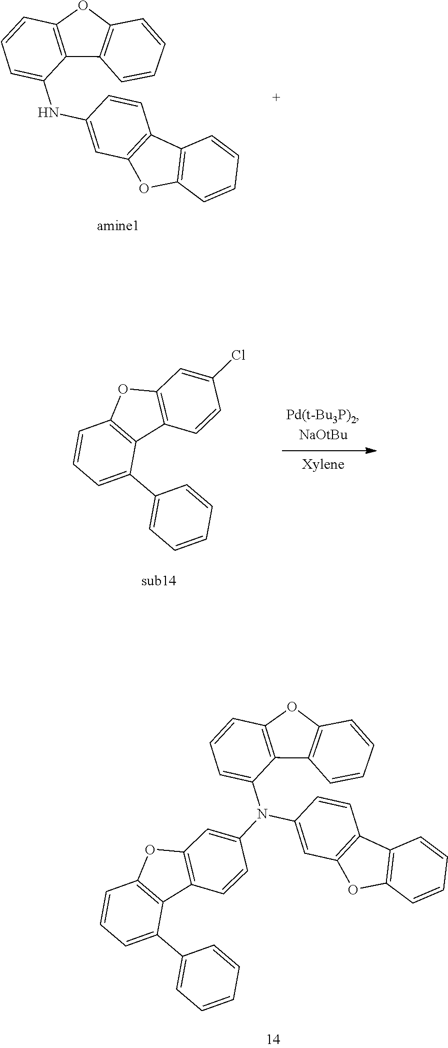 [SMILES: c1ccc2c(c1)oc1cc(Nc3cccc4oc5ccccc5c34)ccc12 ; Clc1ccc2c(c1)oc1cccc(-c3ccccc3)c12 ; c1ccc(-c2cccc3oc4cc(N(c5ccc6c(c5)oc5ccccc56)c5cccc6oc7ccccc7c56)ccc4c23)cc1]

amine1 (15 g, 42.9 mmol), sub14 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 13.5 g of Compound 14. (Yield: 53%, MS: [M+H]+=592)

### Synthesis Example 15

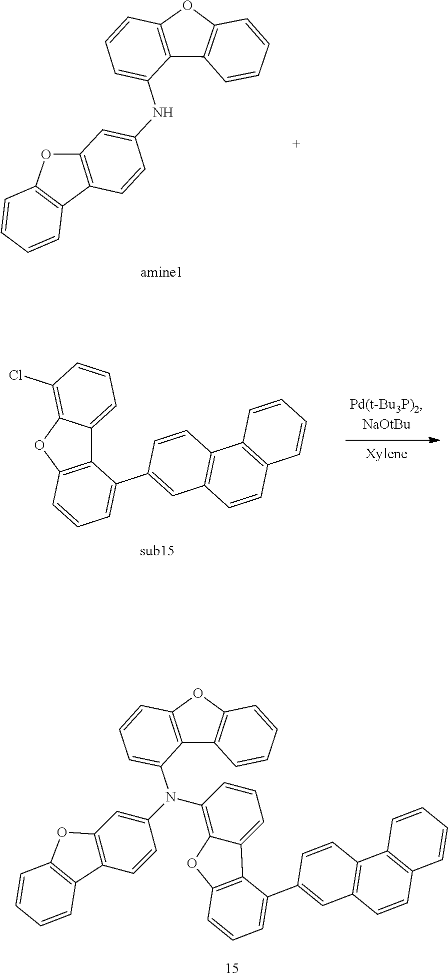 [SMILES: c1ccc2c(c1)oc1cc(Nc3cccc4oc5ccccc5c34)ccc12 ; Clc1cccc2c1oc1cccc(-c3ccc4c(ccc5ccccc54)c3)c12 ; c1ccc2c(c1)ccc1cc(-c3cccc4oc5c(N(c6ccc7c(c6)oc6ccccc67)c6cccc7oc8ccccc8c67)cccc5c34)ccc12]

amine1 (15 g, 42.9 mmol), sub15 (17.1 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.8 g of Compound 15. (Yield: 50%, MS: [M+H]+=692)

### Synthesis Example 16

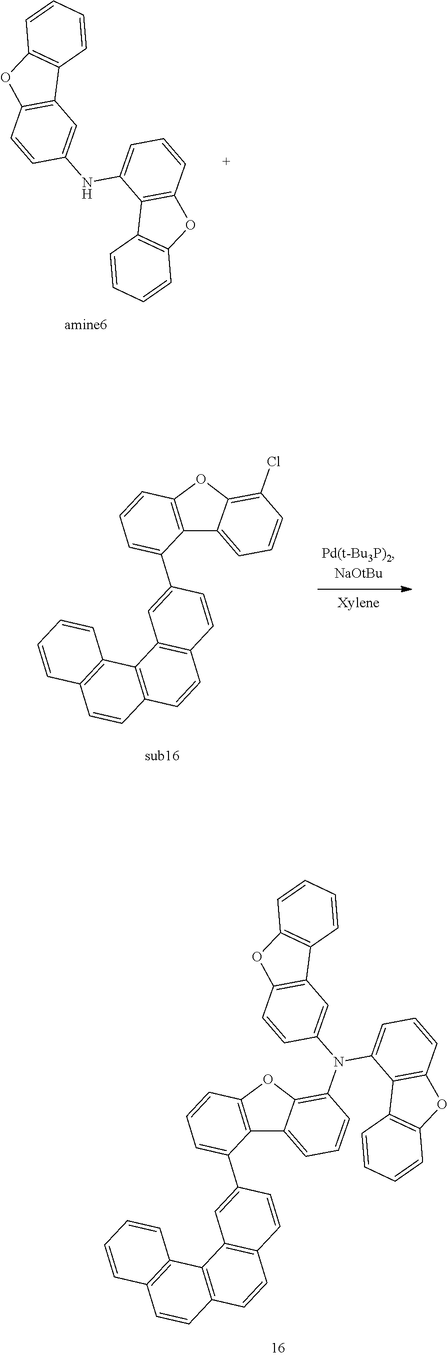 [SMILES: c1ccc2c(c1)oc1ccc(Nc3cccc4oc5ccccc5c34)cc12 ; Clc1cccc2c1oc1cccc(-c3ccc4ccc5ccc6ccccc6c5c4c3)c12 ; c1ccc2c(c1)ccc1ccc3ccc(-c4cccc5oc6c(N(c7ccc8oc9ccccc9c8c7)c7cccc8oc9ccccc9c78)cccc6c45)cc3c12]

amine6 (15 g, 42.9 mmol), sub16 (19.3 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 21 g of Compound 16. (Yield: 66%, MS: [M+H]+=742)

### Synthesis Example 17

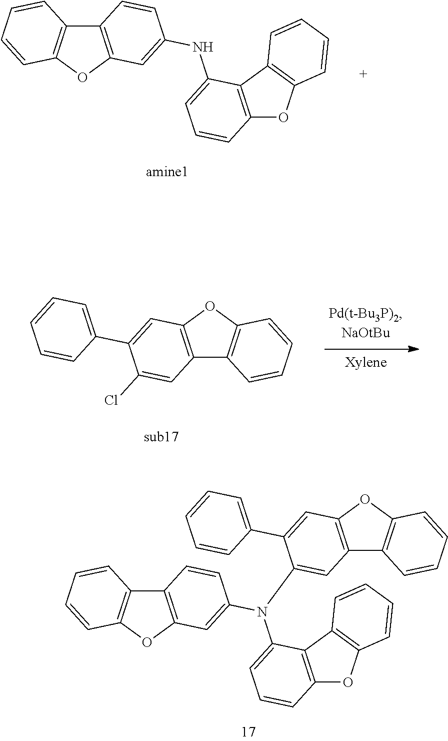 [SMILES: c1ccc2c(c1)oc1cc(Nc3cccc4oc5ccccc5c34)ccc12 ; Clc1cc2c(cc1-c1ccccc1)oc1ccccc12 ; c1ccc(-c2cc3oc4ccccc4c3cc2N(c2ccc3c(c2)oc2ccccc23)c2cccc3oc4ccccc4c23)cc1]

amine1 (15 g, 42.9 mmol), sub17 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 12.9 g of Compound 17. (Yield: 51%, MS: [M+H]+=592)

### Synthesis Example 18

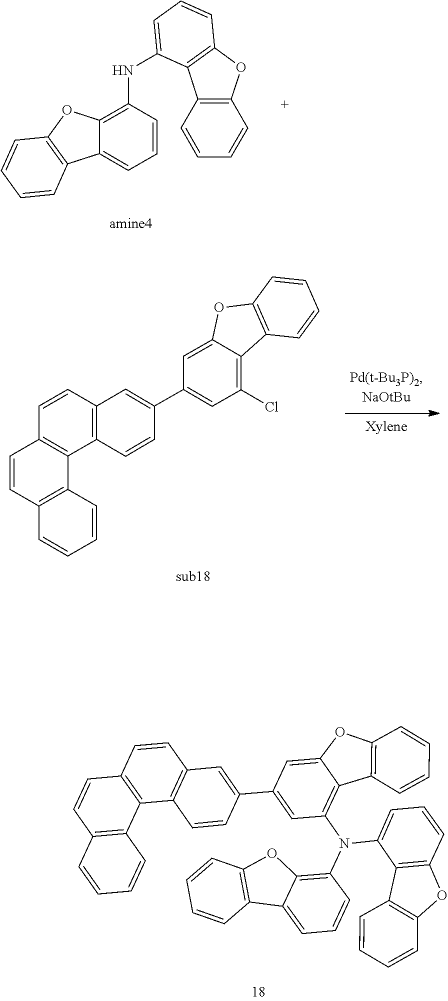 [SMILES: c1ccc2c(c1)oc1c(Nc3cccc4oc5ccccc5c34)cccc12 ; Clc1cc(-c2ccc3c(ccc4ccc5ccccc5c43)c2)cc2oc3ccccc3c12 ; c1ccc2c(c1)ccc1ccc3cc(-c4cc(N(c5cccc6c5oc5ccccc56)c5cccc6oc7ccccc7c56)c5c(c4)oc4ccccc45)ccc3c12]

amine1 (15 g, 42.9 mmol), sub18 (19.3 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 19.7 g of Compound 18. (Yield: 62%, MS: [M+H]+=742)

### Synthesis Example 19

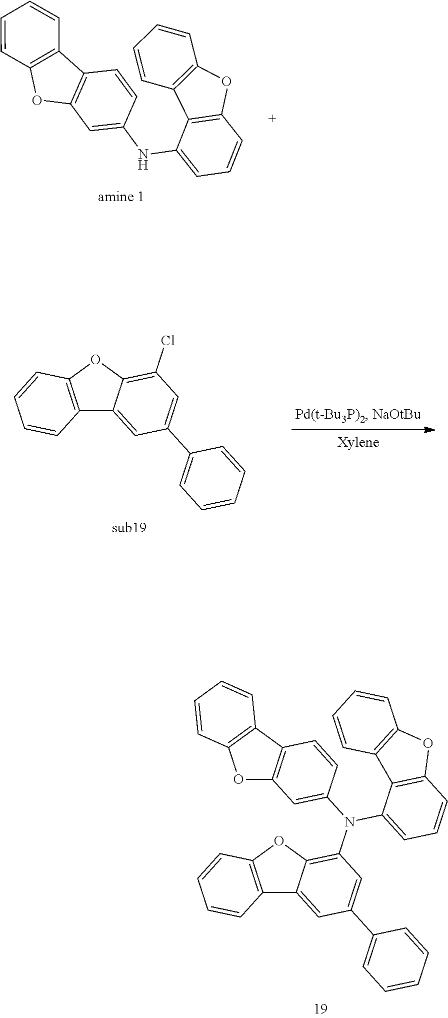 [SMILES: c1ccc2c(c1)oc1cc(Nc3cccc4oc5ccccc5c34)ccc12 ; Clc1cc(-c2ccccc2)cc2c1oc1ccccc12 ; c1ccc(-c2cc(N(c3ccc4c(c3)oc3ccccc34)c3cccc4oc5ccccc5c34)c3oc4ccccc4c3c2)cc1]

amine1 (15 g, 42.9 mmol), sub19 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 13.2 g of Compound 19. (Yield: 52%, MS: [M+H]+=592)

### Synthesis Example 20

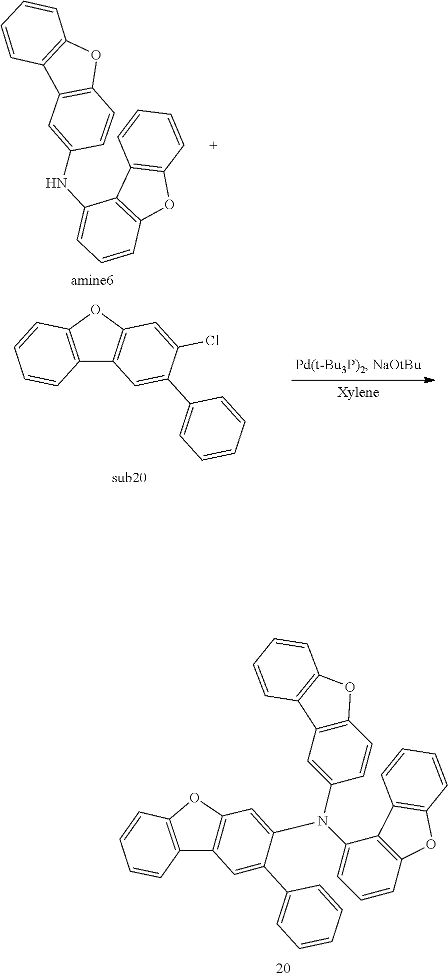 [SMILES: c1ccc2c(c1)oc1ccc(Nc3cccc4oc5ccccc5c34)cc12 ; Clc1cc2oc3ccccc3c2cc1-c1ccccc1 ; c1ccc(-c2cc3c(cc2N(c2ccc4oc5ccccc5c4c2)c2cccc4oc5ccccc5c24)oc2ccccc23)cc1]

amine6 (15 g, 42.9 mmol), sub20 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.5 g of Compound 20. (Yield: 57%, MS: [M+H]+=592)

### Synthesis Example 21

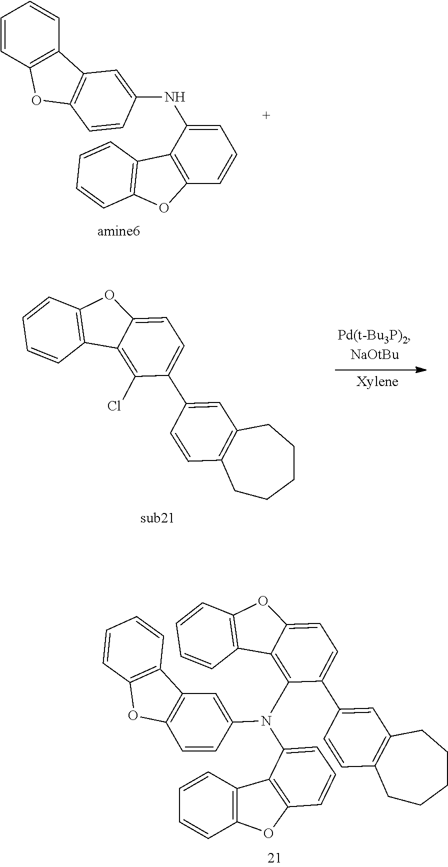 [SMILES: c1ccc2c(c1)oc1ccc(Nc3cccc4oc5ccccc5c34)cc12 ; Clc1c(-c2ccc3c(c2)CCCCC3)ccc2oc3ccccc3c12 ; c1ccc2c(c1)oc1ccc(N(c3cccc4oc5ccccc5c34)c3c(-c4ccc5c(c4)CCCCC5)ccc4oc5ccccc5c34)cc12]

amine6 (15 g, 42.9 mmol), sub21 (15.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 18.4 g of Compound 21. (Yield: 65%, MS: [M+H]+=660)

### Synthesis Example 22

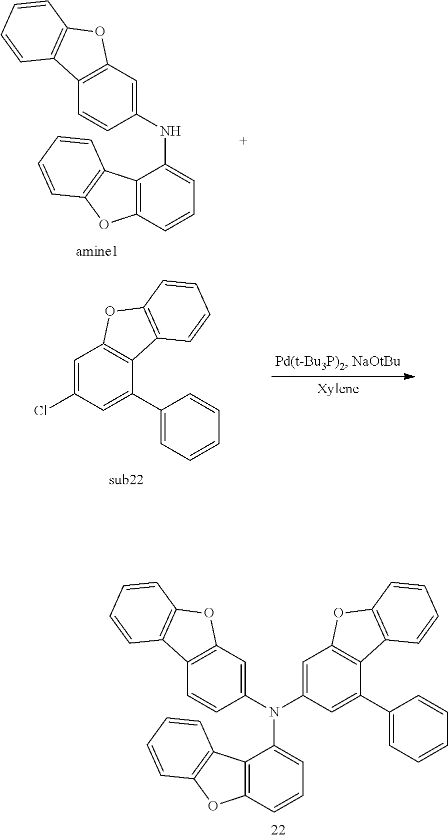 [SMILES: c1ccc2c(c1)oc1cc(Nc3cccc4oc5ccccc5c34)ccc12 ; Clc1cc(-c2ccccc2)c2c(c1)oc1ccccc12 ; c1ccc(-c2cc(N(c3ccc4c(c3)oc3ccccc34)c3cccc4oc5ccccc5c34)cc3oc4ccccc4c23)cc1]

amine1 (15 g, 42.9 mmol), sub22 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 13.2 g of Compound 22. (Yield: 52%, MS: [M+H]+=592)

### Synthesis Example 23

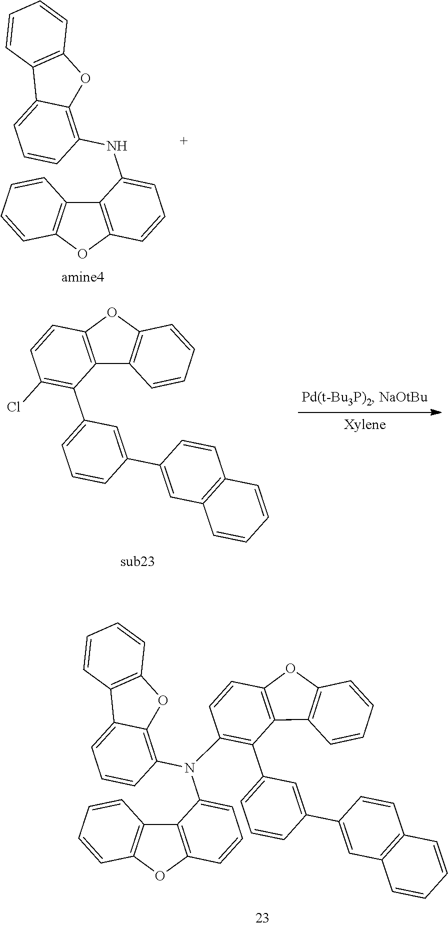 [SMILES: c1ccc2c(c1)oc1c(Nc3cccc4oc5ccccc5c34)cccc12 ; Clc1ccc2oc3ccccc3c2c1-c1cccc(-c2ccc3ccccc3c2)c1 ; c1cc(-c2ccc3ccccc3c2)cc(-c2c(N(c3cccc4c3oc3ccccc34)c3cccc4oc5ccccc5c34)ccc3oc4ccccc4c23)c1]

amine4 (15 g, 42.9 mmol), sub23 (18.3 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15.4 g of Compound 23. (Yield: 50%, MS: [M+H]+=718)

### Synthesis Example 24

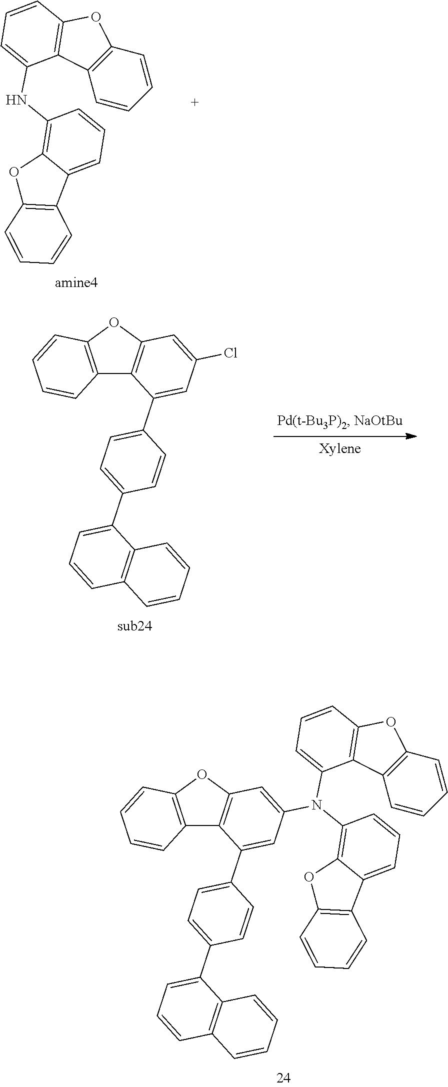 [SMILES: c1ccc2c(c1)oc1c(Nc3cccc4oc5ccccc5c34)cccc12 ; Clc1cc(-c2ccc(-c3cccc4ccccc34)cc2)c2c(c1)oc1ccccc12 ; c1ccc2c(-c3ccc(-c4cc(N(c5cccc6c5oc5ccccc56)c5cccc6oc7ccccc7c56)cc5oc6ccccc6c45)cc3)cccc2c1]

amine4 (15 g, 42.9 mmol), sub24 (18.3 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 18.8 g of Compound 24. (Yield: 61%, MS: [M+H]+=718)

### Synthesis Example 25

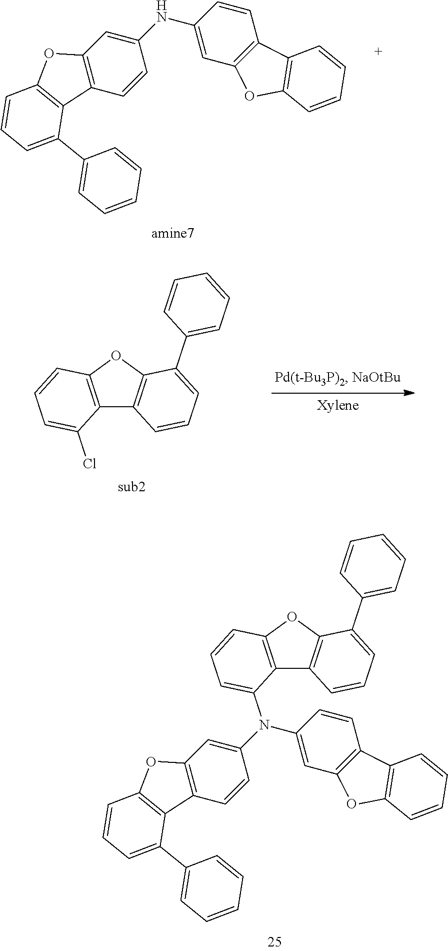 [SMILES: c1ccc(-c2cccc3oc4cc(Nc5ccc6c(c5)oc5ccccc56)ccc4c23)cc1 ; Clc1cccc2oc3c(-c4ccccc4)cccc3c12 ; c1ccc(-c2cccc3c2oc2cccc(N(c4ccc5c(c4)oc4ccccc45)c4ccc5c(c4)oc4cccc(-c6ccccc6)c45)c23)cc1]

amine7 (15 g, 35.3 mmol), sub2 (10.3 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15.1 g of Compound 25. (Yield: 64%, MS: [M+H]+=668)

### Synthesis Example 26

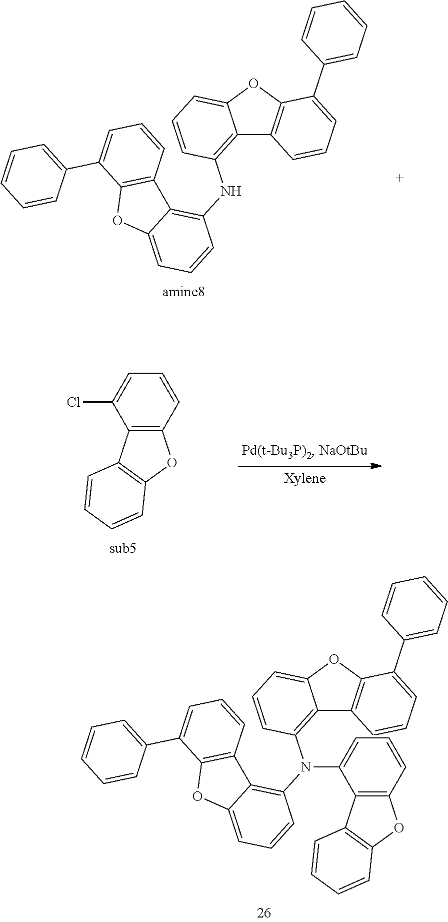 [SMILES: c1ccc(-c2cccc3c2oc2cccc(Nc4cccc5oc6c(-c7ccccc7)cccc6c45)c23)cc1 ; Clc1cccc2oc3ccccc3c12 ; c1ccc(-c2cccc3c2oc2cccc(N(c4cccc5oc6ccccc6c45)c4cccc5oc6c(-c7ccccc7)cccc6c45)c23)cc1]

amine8 (15 g, 29.9 mmol), sub5 (6.4 g, 31.4 mmol) and sodium tert-butoxide (4.3 g, 44.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 11.2 g of Compound 26. (Yield: 56%, MS: [M+H]+=668)

### Synthesis Example 27

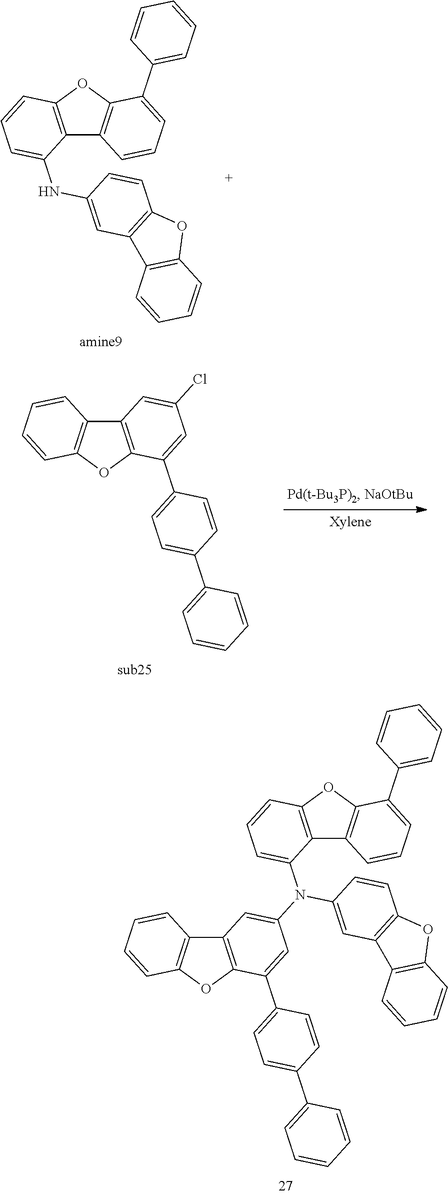 [SMILES: c1ccc(-c2cccc3c2oc2cccc(Nc4ccc5oc6ccccc6c5c4)c23)cc1 ; Clc1cc(-c2ccc(-c3ccccc3)cc2)c2oc3ccccc3c2c1 ; c1ccc(-c2ccc(-c3cc(N(c4ccc5oc6ccccc6c5c4)c4cccc5oc6c(-c7ccccc7)cccc6c45)cc4c3oc3ccccc34)cc2)cc1]

amine9 (15 g, 35.3 mmol), sub25 (13.1 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15.5 g of Compound 27. (Yield: 59%, MS: [M+H]+=744)

### Synthesis Example 28

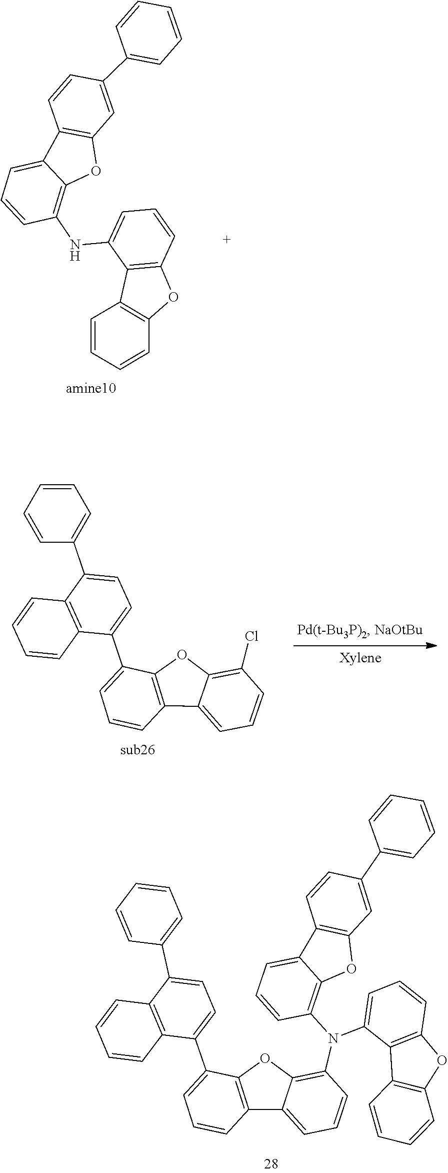 [SMILES: c1ccc(-c2ccc3c(c2)oc2c(Nc4cccc5oc6ccccc6c45)cccc23)cc1 ; Clc1cccc2c1oc1c(-c3ccc(-c4ccccc4)c4ccccc34)cccc12 ; c1ccc(-c2ccc3c(c2)oc2c(N(c4cccc5c4oc4c(-c6ccc(-c7ccccc7)c7ccccc67)cccc45)c4cccc5oc6ccccc6c45)cccc23)cc1]

amine10 (15 g, 35.3 mmol), sub26 (15 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14 g of Compound 28. (Yield: 50%, MS: [M+H]+=794)

### Synthesis Example 29

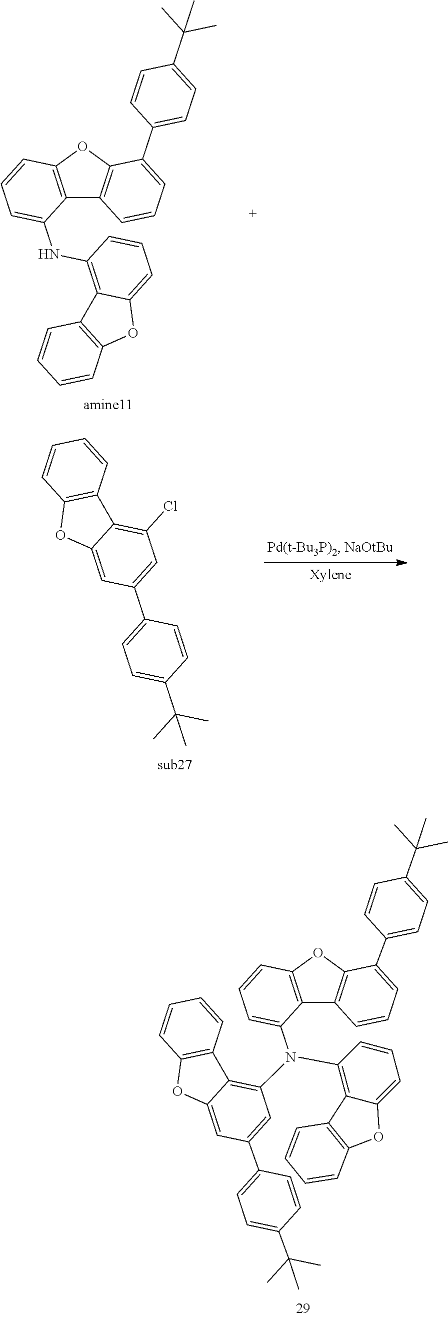 [SMILES: CC(C)(C)c1ccc(-c2cccc3c2oc2cccc(Nc4cccc5oc6ccccc6c45)c23)cc1 ; CC(C)(C)c1ccc(-c2cc(Cl)c3c(c2)oc2ccccc23)cc1 ; CC(C)(C)c1ccc(-c2cc(N(c3cccc4oc5ccccc5c34)c3cccc4oc5c(-c6ccc(C(C)(C)C)cc6)cccc5c34)c3c(c2)oc2ccccc23)cc1]

amine11 (15 g, 31.1 mmol), sub27 (11 g, 32.7 mmol) and sodium tert-butoxide (4.5 g, 46.7 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.3 g of Compound 29. (Yield: 59%, MS: [M+H]+=780)

### Synthesis Example 30

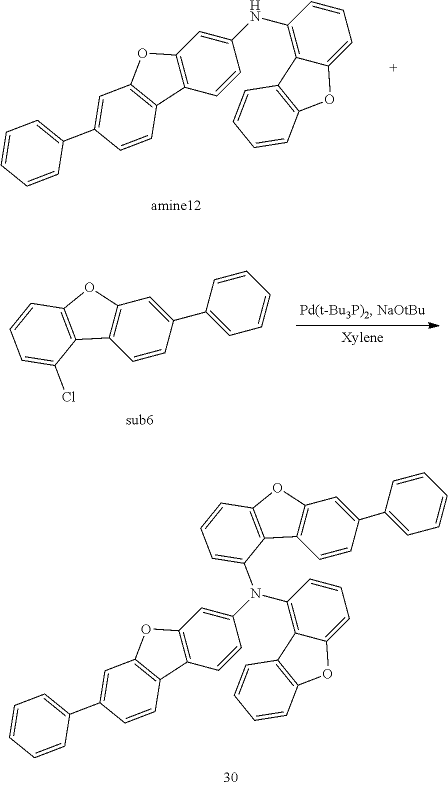 [SMILES: c1ccc(-c2ccc3c(c2)oc2cc(Nc4cccc5oc6ccccc6c45)ccc23)cc1 ; Clc1cccc2oc3cc(-c4ccccc4)ccc3c12 ; c1ccc(-c2ccc3c(c2)oc2cc(N(c4cccc5oc6ccccc6c45)c4cccc5oc6cc(-c7ccccc7)ccc6c45)ccc23)cc1]

amine12 (15 g, 35.3 mmol), sub6 (10.3 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15.5 g of Compound 30. (Yield: 66%, MS: [M+H]+=668)

### Synthesis Example 31

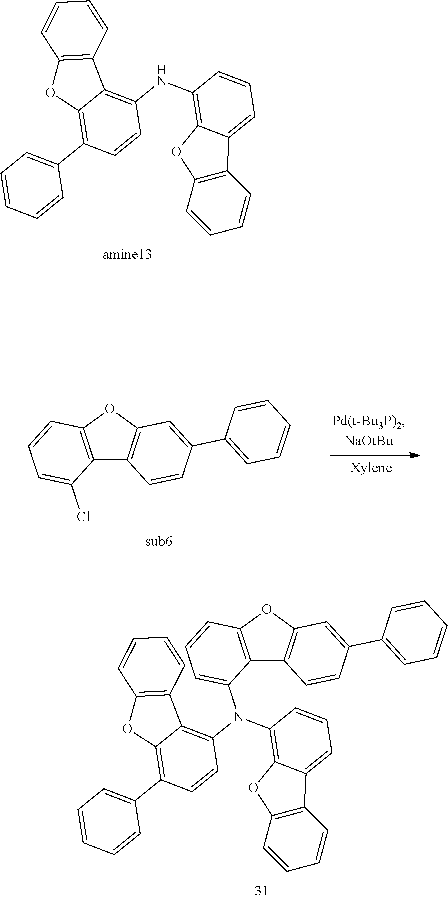 [SMILES: c1ccc(-c2ccc(Nc3cccc4c3oc3ccccc34)c3c2oc2ccccc23)cc1 ; Clc1cccc2oc3cc(-c4ccccc4)ccc3c12 ; c1ccc(-c2ccc3c(c2)oc2cccc(N(c4cccc5c4oc4ccccc45)c4ccc(-c5ccccc5)c5oc6ccccc6c45)c23)cc1]

amine13 (15 g, 35.3 mmol), sub6 (10.3 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 12.2 g of Compound 31. (Yield: 52%, MS: [M+H]+=668)

### Synthesis Example 32

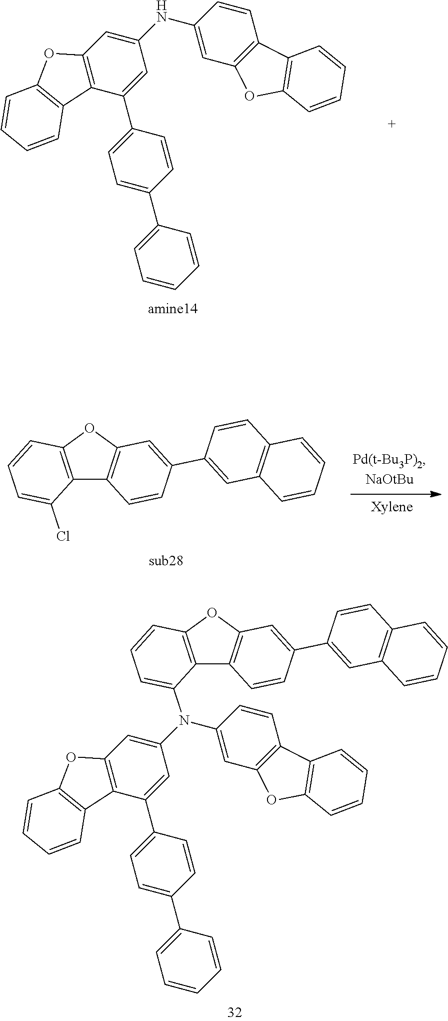 [SMILES: c1ccc(-c2ccc(-c3cc(Nc4ccc5c(c4)oc4ccccc45)cc4oc5ccccc5c34)cc2)cc1 ; Clc1cccc2oc3cc(-c4ccc5ccccc5c4)ccc3c12 ; c1ccc(-c2ccc(-c3cc(N(c4ccc5c(c4)oc4ccccc45)c4cccc5oc6cc(-c7ccc8ccccc8c7)ccc6c45)cc4oc5ccccc5c34)cc2)cc1]

amine14 (15 g, 29.9 mmol), sub28 (10.3 g, 31.4 mmol) and sodium tert-butoxide (4.3 g, 44.9 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 12.3 g of Compound 32. (Yield: 52%, MS: [M+H]+=794)

### Synthesis Example 33

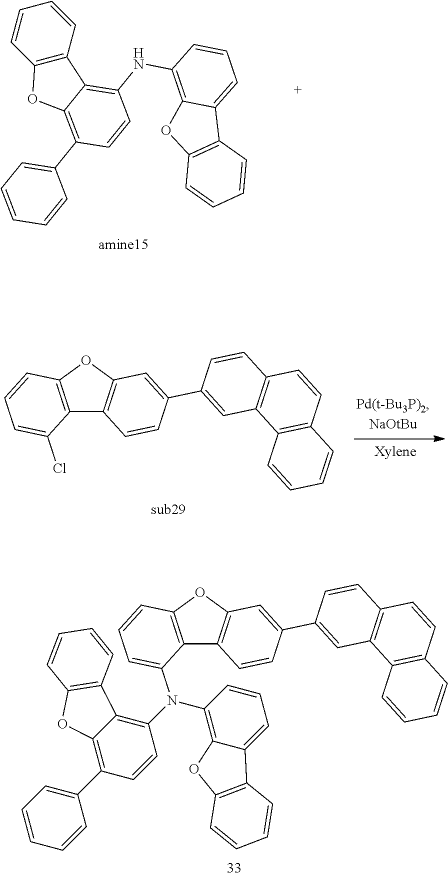 [SMILES: c1ccc(-c2ccc(Nc3cccc4c3oc3ccccc34)c3c2oc2ccccc23)cc1 ; Clc1cccc2oc3cc(-c4ccc5ccc6ccccc6c5c4)ccc3c12 ; c1ccc(-c2ccc(N(c3cccc4c3oc3ccccc34)c3cccc4oc5cc(-c6ccc7ccc8ccccc8c7c6)ccc5c34)c3c2oc2ccccc23)cc1]

amine15 (15 g, 35.3 mmol), sub29 (14 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15.7 g of Compound 33. (Yield: 58%, MS: [M+H]+=768)

### Synthesis Example 34

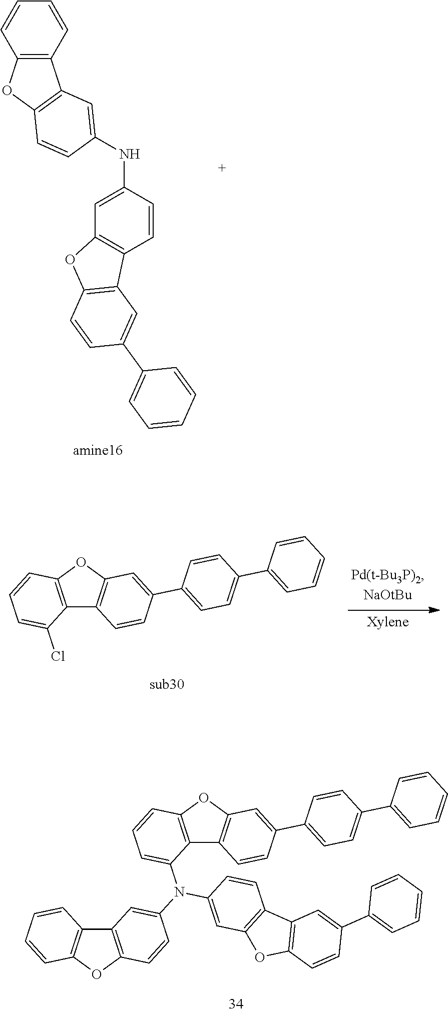 [SMILES: c1ccc(-c2ccc3oc4cc(Nc5ccc6oc7ccccc7c6c5)ccc4c3c2)cc1 ; Clc1cccc2oc3cc(-c4ccc(-c5ccccc5)cc4)ccc3c12 ; c1ccc(-c2ccc(-c3ccc4c(c3)oc3cccc(N(c5ccc6c(c5)oc5ccc(-c7ccccc7)cc56)c5ccc6oc7ccccc7c6c5)c34)cc2)cc1]

amine16 (15 g, 35.3 mmol), sub30 (13.1 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 13.4 g of Compound 34. (Yield: 51%, MS: [M+H]+=744)

### Synthesis Example 35

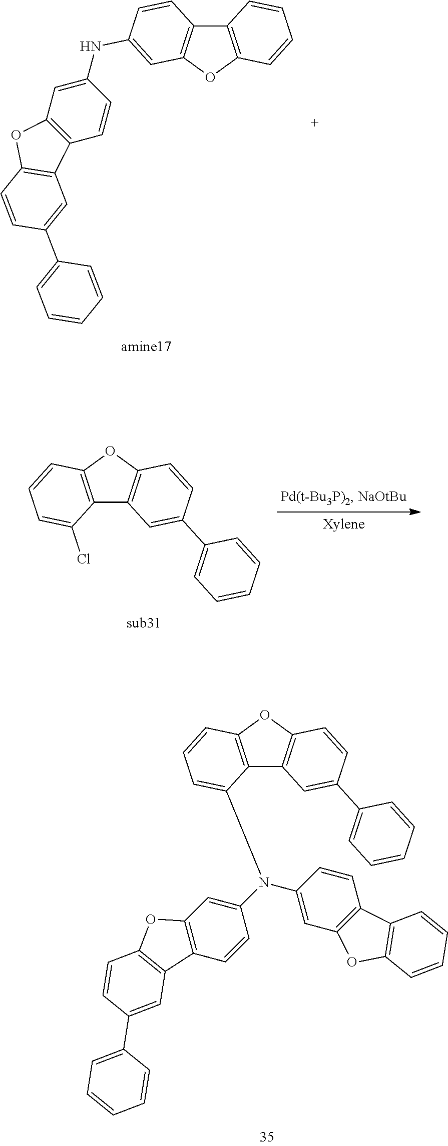 [SMILES: c1ccc(-c2ccc3oc4cc(Nc5ccc6c(c5)oc5ccccc56)ccc4c3c2)cc1 ; Clc1cccc2oc3ccc(-c4ccccc4)cc3c12 ; c1ccc(-c2ccc3oc4cc(N(c5ccc6c(c5)oc5ccccc56)c5cccc6oc7ccc(-c8ccccc8)cc7c56)ccc4c3c2)cc1]

amine17 (15 g, 35.3 mmol), sub31 (10.3 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 12.9 g of Compound 35. (Yield: 55%, MS: [M+H]+=688)

### Synthesis Example 36

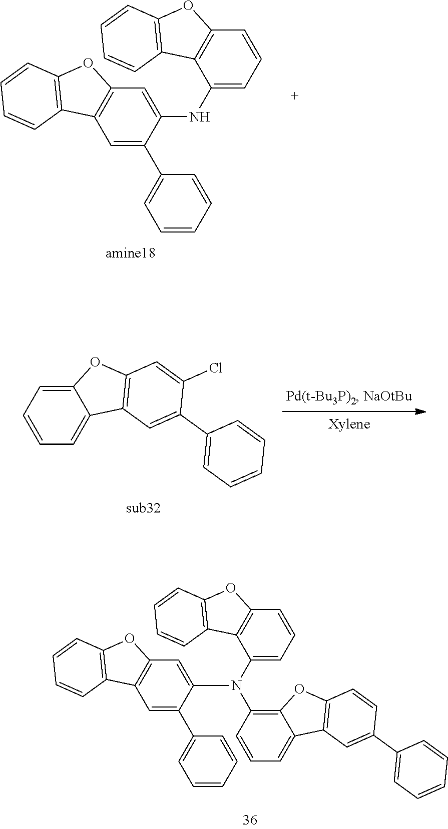 [SMILES: c1ccc(-c2cc3c(cc2Nc2cccc4oc5ccccc5c24)oc2ccccc23)cc1 ; Clc1cc2oc3ccccc3c2cc1-c1ccccc1 ; c1ccc(-c2ccc3oc4c(N(c5cc6oc7ccccc7c6cc5-c5ccccc5)c5cccc6oc7ccccc7c56)cccc4c3c2)cc1]

amine18 (15 g, 35.3 mmol), sub32 (10.3 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 12.9 g of Compound 36. (Yield: 55%, MS: [M+H]+=688)

### Synthesis Example 37

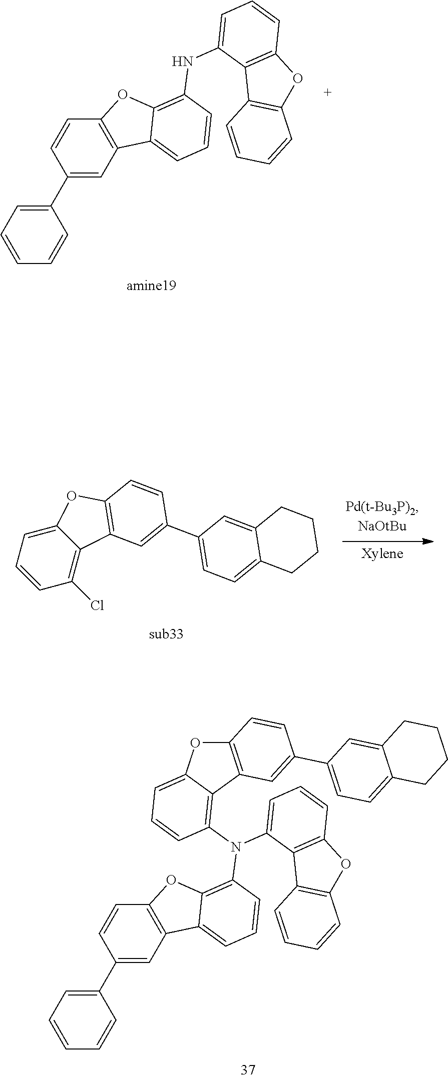 [SMILES: c1ccc(-c2ccc3oc4c(Nc5cccc6oc7ccccc7c56)cccc4c3c2)cc1 ; Clc1cccc2oc3ccc(-c4ccc5c(c4)CCCC5)cc3c12 ; c1ccc(-c2ccc3oc4c(N(c5cccc6oc7ccccc7c56)c5cccc6oc7ccc(-c8ccc9c(c8)CCCC9)cc7c56)cccc4c3c2)cc1]

amine19 (15 g, 3.5 mmol), sub33 (1.2 g, 3.7 mmol) and sodium tert-butoxide (0.5 g, 5.3 mmol) were added to 300 ml of xylene under a nitrogen atmosphere, and mixture was stirred and refluxed. Then, bis(tri-tert-butylphosphine)palladium(0) (0 g, 0 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 1.8 g of Compound 37. (Yield: 69%, MS: [M+H]+=722)

### Synthesis Example 38

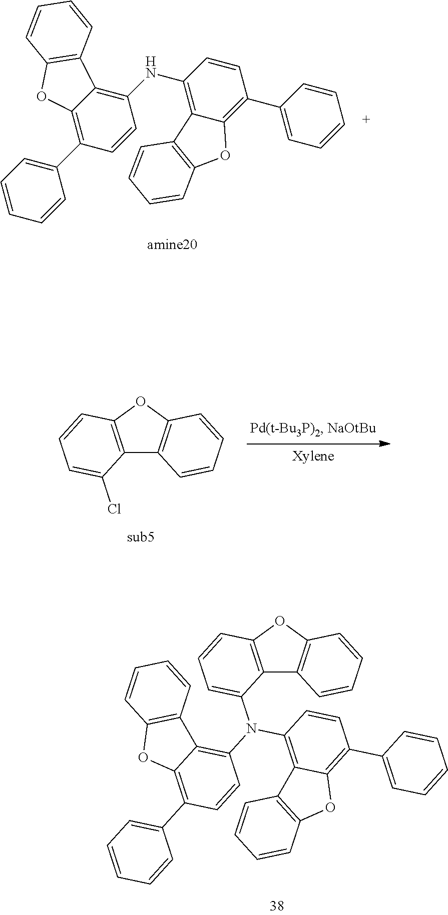 [SMILES: c1ccc(-c2ccc(Nc3ccc(-c4ccccc4)c4oc5ccccc5c34)c3c2oc2ccccc23)cc1 ; Clc1cccc2oc3ccccc3c12 ; c1ccc(-c2ccc(N(c3cccc4oc5ccccc5c34)c3ccc(-c4ccccc4)c4oc5ccccc5c34)c3c2oc2ccccc23)cc1]

amine20 (15 g, 29.9 mmol), sub5 (6.4 g, 31.4 mmol) and sodium tert-butoxide (4.3 g, 44.9 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 13.4 g of Compound 38. (Yield: 67%, MS: [M+H]+=688)

### Synthesis Example 39

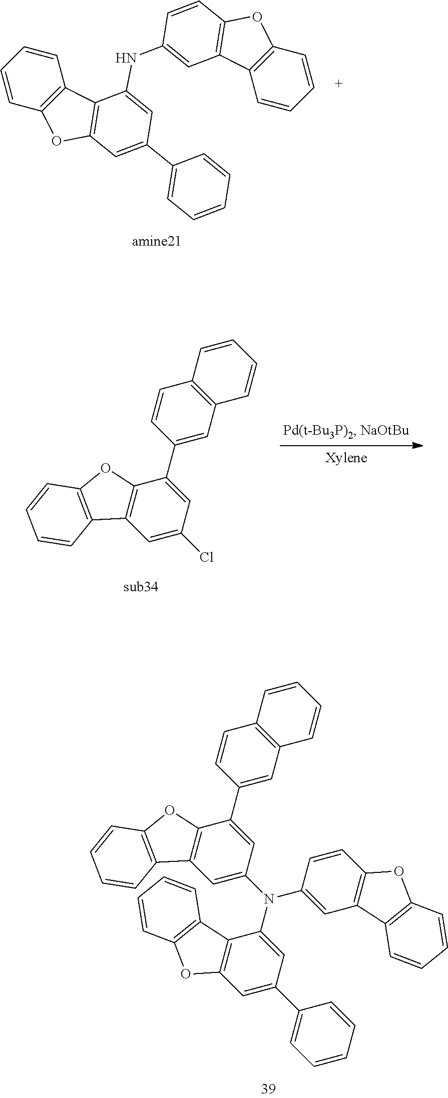 [SMILES: c1ccc(-c2cc(Nc3ccc4oc5ccccc5c4c3)c3c(c2)oc2ccccc23)cc1 ; Clc1cc(-c2ccc3ccccc3c2)c2oc3ccccc3c2c1 ; c1ccc(-c2cc(N(c3ccc4oc5ccccc5c4c3)c3cc(-c4ccc5ccccc5c4)c4oc5ccccc5c4c3)c3c(c2)oc2ccccc23)cc1]

amine21 (15 g, 35.3 mmol), sub34 (12.2 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 16.7 g of Compound 39. (Yield: 66%, MS: [M+H]+=718)

### Synthesis Example 40

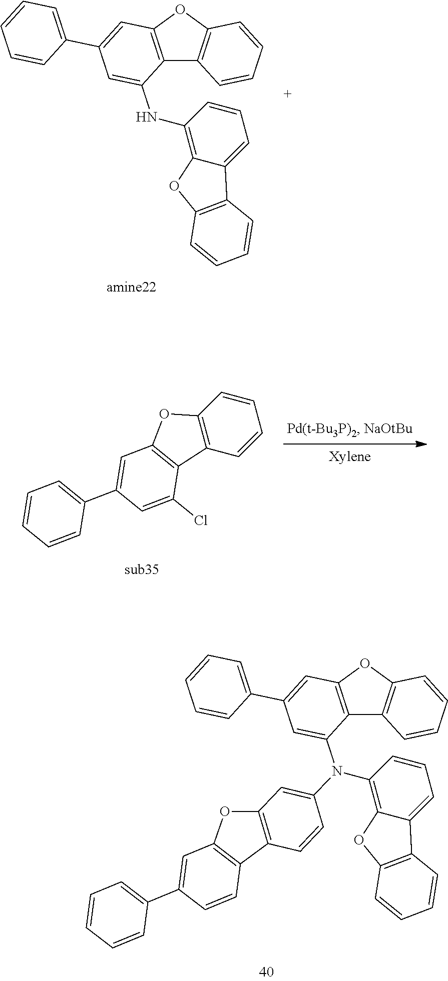 [SMILES: c1ccc(-c2cc(Nc3cccc4c3oc3ccccc34)c3c(c2)oc2ccccc23)cc1 ; Clc1cc(-c2ccccc2)cc2oc3ccccc3c12 ; c1ccc(-c2ccc3c(c2)oc2cc(N(c4cccc5c4oc4ccccc45)c4cc(-c5ccccc5)cc5oc6ccccc6c45)ccc23)cc1]

amine22 (15 g, 35.3 mmol), sub35 (10.3 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.6 g of Compound 40. (Yield: 62%, MS: [M+H]+=668)

### Synthesis Example 41

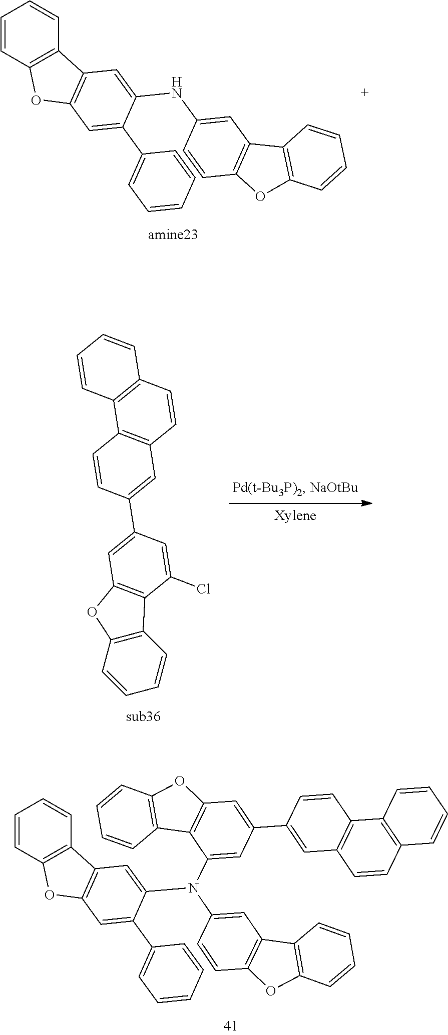 [SMILES: c1ccc(-c2cc3oc4ccccc4c3cc2Nc2ccc3oc4ccccc4c3c2)cc1 ; Clc1cc(-c2ccc3c(ccc4ccccc43)c2)cc2oc3ccccc3c12 ; c1ccc(-c2cc3oc4ccccc4c3cc2N(c2ccc3oc4ccccc4c3c2)c2cc(-c3ccc4c(ccc5ccccc54)c3)cc3oc4ccccc4c23)cc1]

amine23 (15 g, 35.3 mmol), sub36 (14 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.9 g of Compound 41. (Yield: 55%, MS: [M+H]+=768)

### Synthesis Example 42

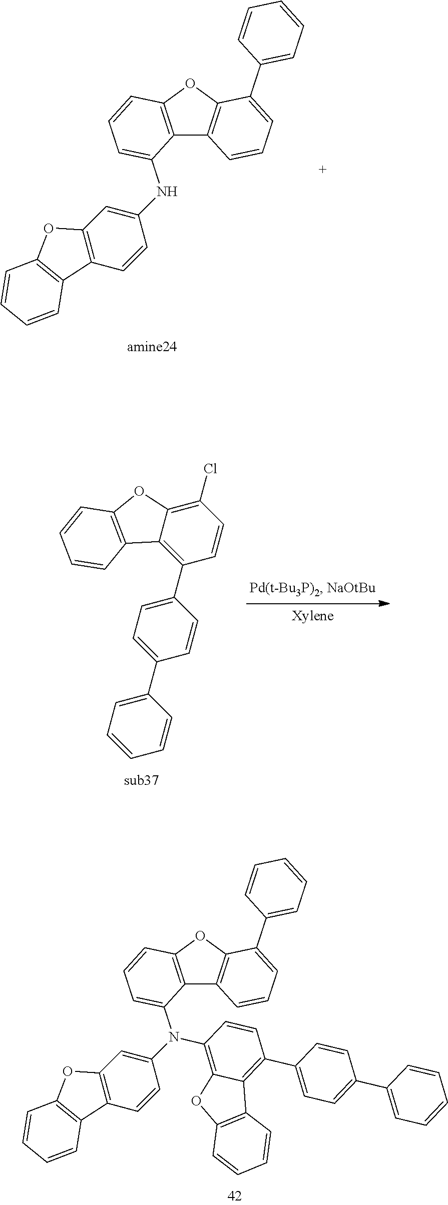 [SMILES: c1ccc(-c2cccc3c2oc2cccc(Nc4ccc5c(c4)oc4ccccc45)c23)cc1 ; Clc1ccc(-c2ccc(-c3ccccc3)cc2)c2c1oc1ccccc12 ; c1ccc(-c2ccc(-c3ccc(N(c4ccc5c(c4)oc4ccccc45)c4cccc5oc6c(-c7ccccc7)cccc6c45)c4oc5ccccc5c34)cc2)cc1]

amine24 (15 g, 35.3 mmol), sub37 (13.1 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 16 g of Compound 42. (Yield: 61%, MS: [M+H]+=744)

### Synthesis Example 43

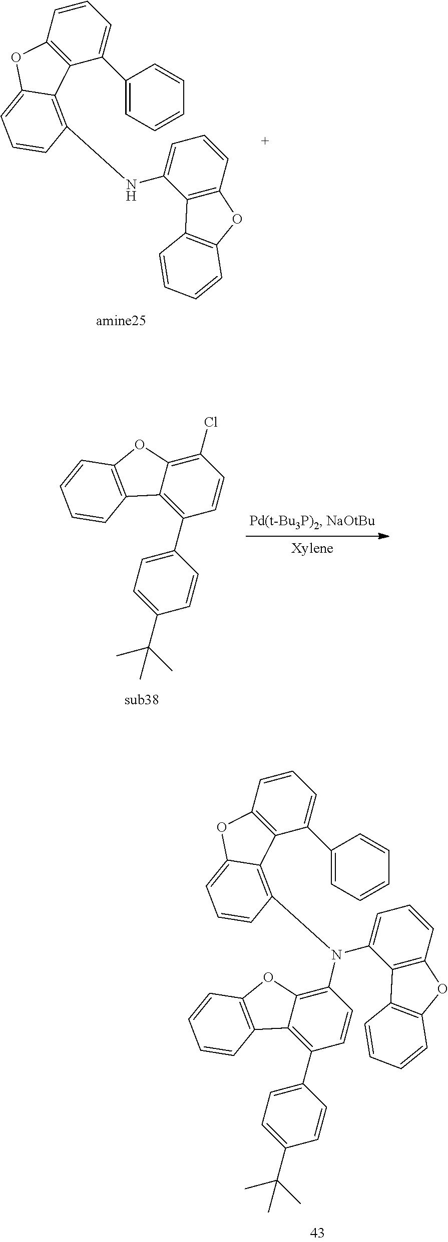 [SMILES: c1ccc(-c2cccc3oc4cccc(Nc5cccc6oc7ccccc7c56)c4c23)cc1 ; CC(C)(C)c1ccc(-c2ccc(Cl)c3oc4ccccc4c23)cc1 ; CC(C)(C)c1ccc(-c2ccc(N(c3cccc4oc5ccccc5c34)c3cccc4oc5cccc(-c6ccccc6)c5c34)c3oc4ccccc4c23)cc1]

amine25 (15 g, 35.3 mmol), sub38 (12.4 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 13.3 g of Compound 43. (Yield: 52%, MS: [M+H]+=724)

### Synthesis Example 44

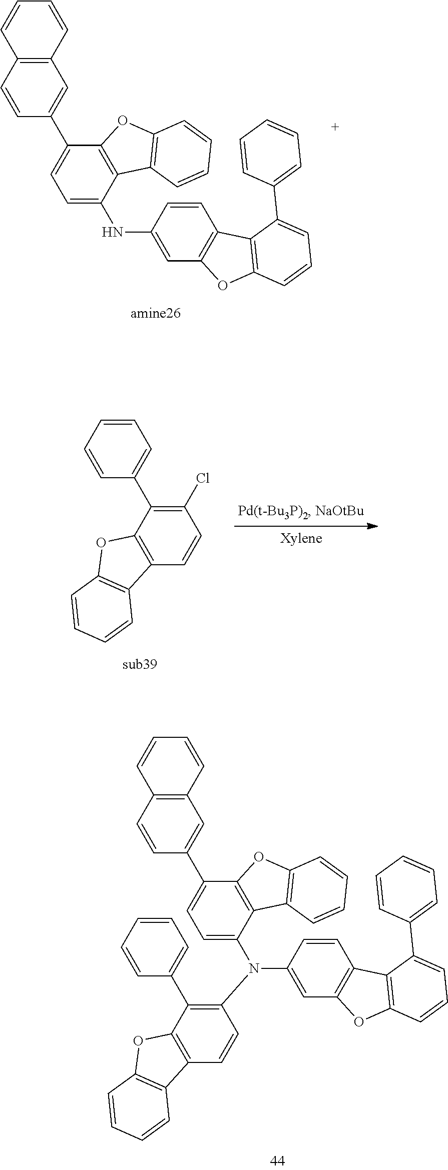 [SMILES: c1ccc(-c2cccc3oc4cc(Nc5ccc(-c6ccc7ccccc7c6)c6oc7ccccc7c56)ccc4c23)cc1 ; Clc1ccc2c(oc3ccccc32)c1-c1ccccc1 ; c1ccc(-c2c(N(c3ccc4c(c3)oc3cccc(-c5ccccc5)c34)c3ccc(-c4ccc5ccccc5c4)c4oc5ccccc5c34)ccc3c2oc2ccccc23)cc1]

amine26 (15 g, 27.2 mmol), sub39 (8 g, 28.6 mmol) and sodium tert-butoxide (3.9 g, 40.8 mmol) were added to 300 ml of xylene under a nitrogen atmosphere, and mixture was stirred and refluxed. Then, bis(tri-tert-butylphosphine)palladium(0) (0.1 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.5 g of Compound 44. (Yield: 67%, MS: [M+H]+=794)

### Synthesis Example 45

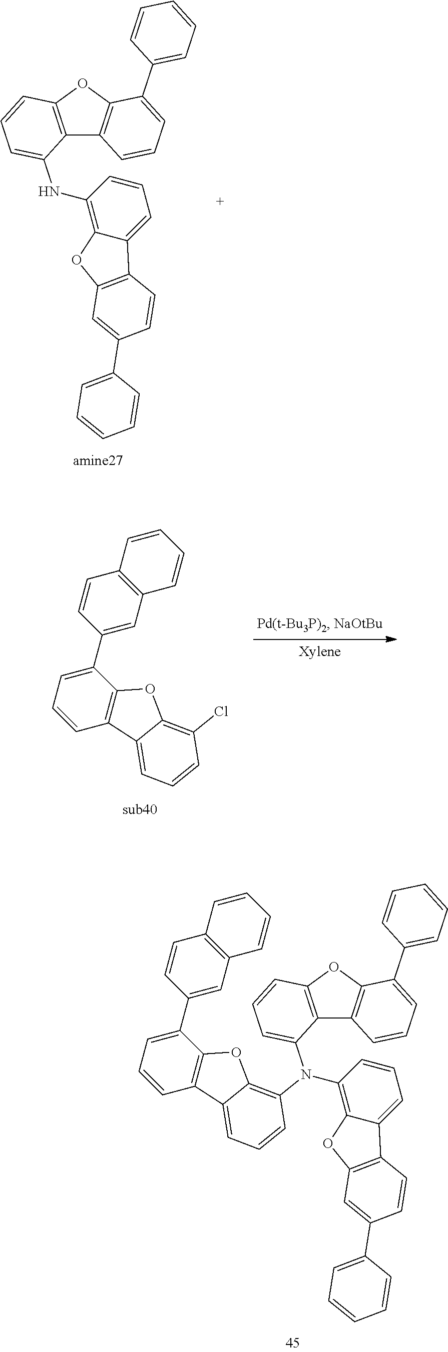 [SMILES: c1ccc(-c2ccc3c(c2)oc2c(Nc4cccc5oc6c(-c7ccccc7)cccc6c45)cccc23)cc1 ; Clc1cccc2c1oc1c(-c3ccc4ccccc4c3)cccc12 ; c1ccc(-c2ccc3c(c2)oc2c(N(c4cccc5c4oc4c(-c6ccc7ccccc7c6)cccc45)c4cccc5oc6c(-c7ccccc7)cccc6c45)cccc23)cc1]

amine27 (15 g, 29.9 mmol), sub40 (10.3 g, 31.4 mmol) and sodium tert-butoxide (4.3 g, 44.9 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 16.6 g of Compound 45. (Yield: 70%, MS: [M+H]+=794)

### Synthesis Example 46

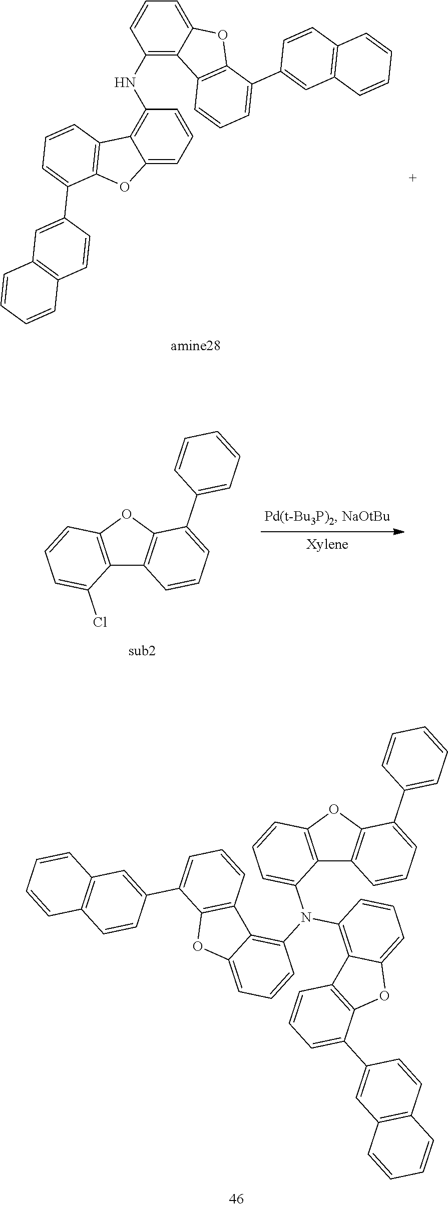 [SMILES: c1ccc2cc(-c3cccc4c3oc3cccc(Nc5cccc6oc7c(-c8ccc9ccccc9c8)cccc7c56)c34)ccc2c1 ; Clc1cccc2oc3c(-c4ccccc4)cccc3c12 ; c1ccc(-c2cccc3c2oc2cccc(N(c4cccc5oc6c(-c7ccc8ccccc8c7)cccc6c45)c4cccc5oc6c(-c7ccc8ccccc8c7)cccc6c45)c23)cc1]

amine28 (15 g, 24.9 mmol), sub2 (7.3 g, 26.2 mmol) and sodium tert-butoxide (3.6 g, 37.4 mmol) were added to 300 ml of xylene under a nitrogen atmosphere, and mixture was stirred and refluxed. Then, bis(tri-tert-butylphosphine)palladium(0) (0.1 g, 0.2 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 12.8 g of Compound 46. (Yield: 61%, MS: [M+H]+=844)

### Synthesis Example 47

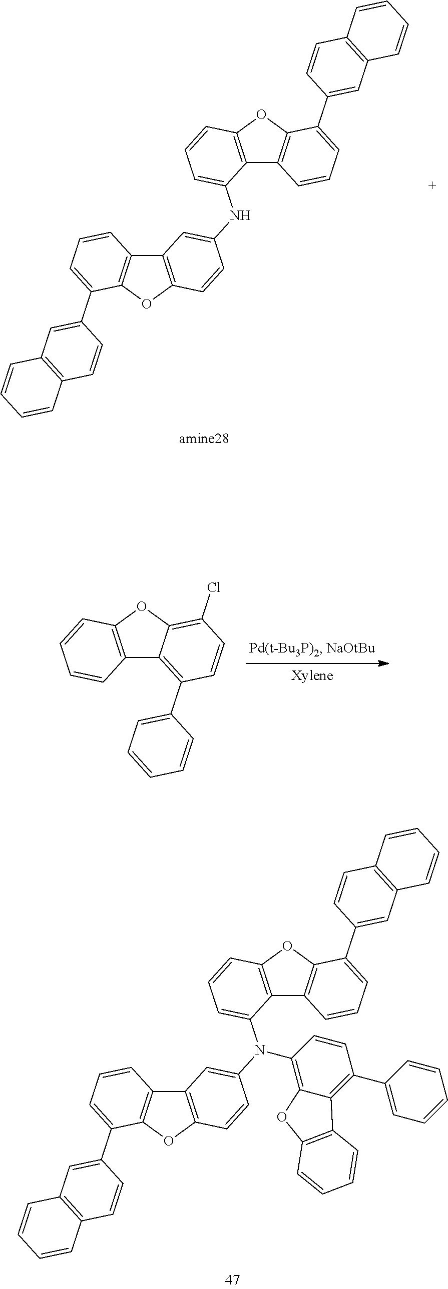 [SMILES: c1ccc2cc(-c3cccc4c3oc3ccc(Nc5cccc6oc7c(-c8ccc9ccccc9c8)cccc7c56)cc34)ccc2c1 ; Clc1ccc(-c2ccccc2)c2c1oc1ccccc12 ; c1ccc(-c2ccc(N(c3ccc4oc5c(-c6ccc7ccccc7c6)cccc5c4c3)c3cccc4oc5c(-c6ccc7ccccc7c6)cccc5c34)c3oc4ccccc4c23)cc1]

amine28 (15 g, 24.9 mmol), sub41 (7.3 g, 26.2 mmol) and sodium tert-butoxide (3.6 g, 37.4 mmol) were added to 300 ml of xylene under a nitrogen atmosphere, and mixture was stirred and refluxed. Then, bis(tri-tert-butylphosphine)palladium(0) (0.1 g, 0.2 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 11.1 g of Compound 47. (Yield: 53%, MS: [M+H]+=844)

### Synthesis Example 48

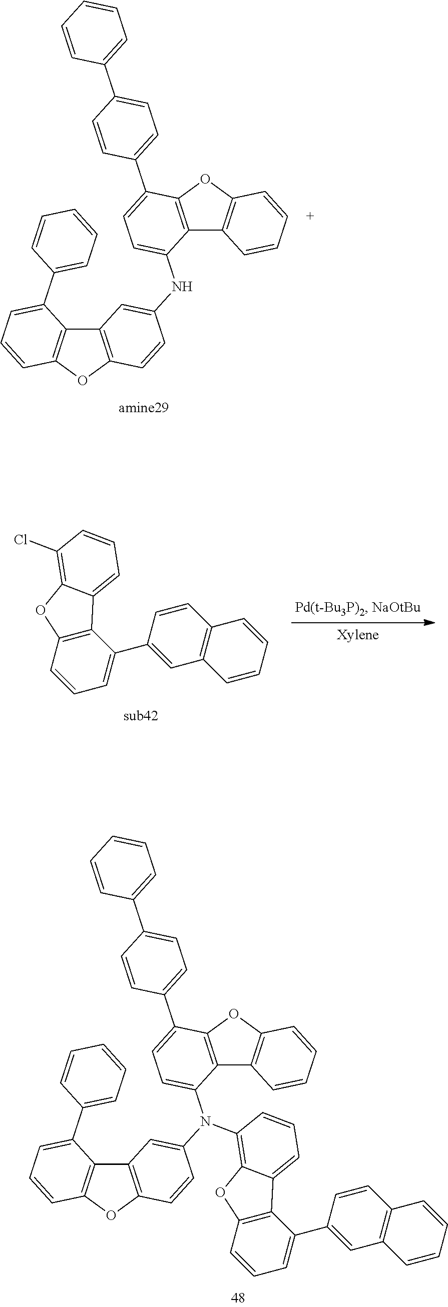 [SMILES: c1ccc(-c2ccc(-c3ccc(Nc4ccc5oc6cccc(-c7ccccc7)c6c5c4)c4c3oc3ccccc34)cc2)cc1 ; Clc1cccc2c1oc1cccc(-c3ccc4ccccc4c3)c12 ; c1ccc(-c2ccc(-c3ccc(N(c4ccc5oc6cccc(-c7ccccc7)c6c5c4)c4cccc5c4oc4cccc(-c6ccc7ccccc7c6)c45)c4c3oc3ccccc34)cc2)cc1]

amine29 (15 g, 26 mmol), sub42 (9 g, 27.3 mmol) and sodium tert-butoxide (3.7 g, 38.9 mmol) were added to 300 ml of xylene under a nitrogen atmosphere, and mixture was stirred and refluxed. Then, bis(tri-tert-butylphosphine)palladium(0) (0.1 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 12.2 g of Compound 48. (Yield: 54%, MS: [M+H]+=870)

### Synthesis Example 49

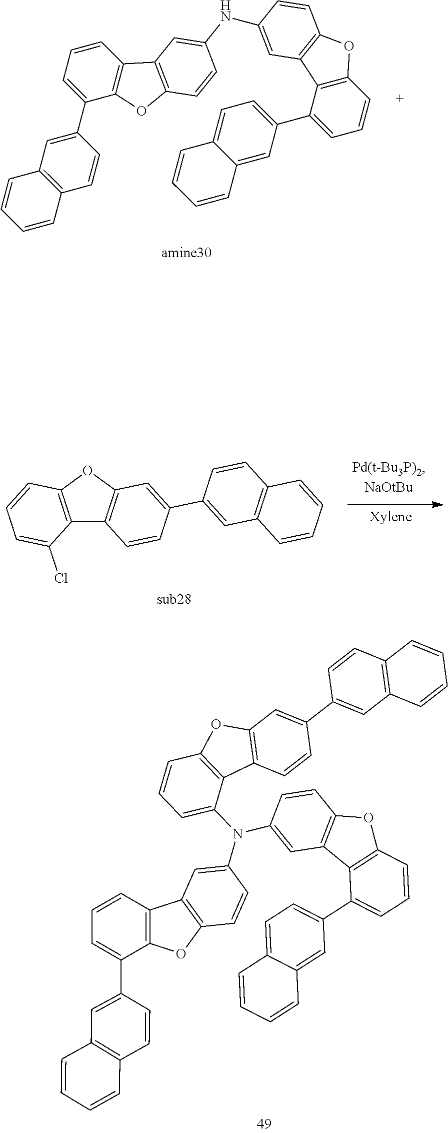 [SMILES: c1ccc2cc(-c3cccc4c3oc3ccc(Nc5ccc6oc7cccc(-c8ccc9ccccc9c8)c7c6c5)cc34)ccc2c1 ; Clc1cccc2oc3cc(-c4ccc5ccccc5c4)ccc3c12 ; c1ccc2cc(-c3ccc4c(c3)oc3cccc(N(c5ccc6oc7c(-c8ccc9ccccc9c8)cccc7c6c5)c5ccc6oc7cccc(-c8ccc9ccccc9c8)c7c6c5)c34)ccc2c1]

amine30 (15 g, 24.9 mmol), sub28 (8.6 g, 26.2 mmol) and sodium tert-butoxide (3.6 g, 37.4 mmol) were added to 300 ml of xylene under a nitrogen atmosphere, and mixture was stirred and refluxed. Then, bis(tri-tert-butylphosphine)palladium(0) (0.1 g, 0.2 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 13.6 g of Compound 49. (Yield: 61%, MS: [M+H]+=894)

### Synthesis Example 50

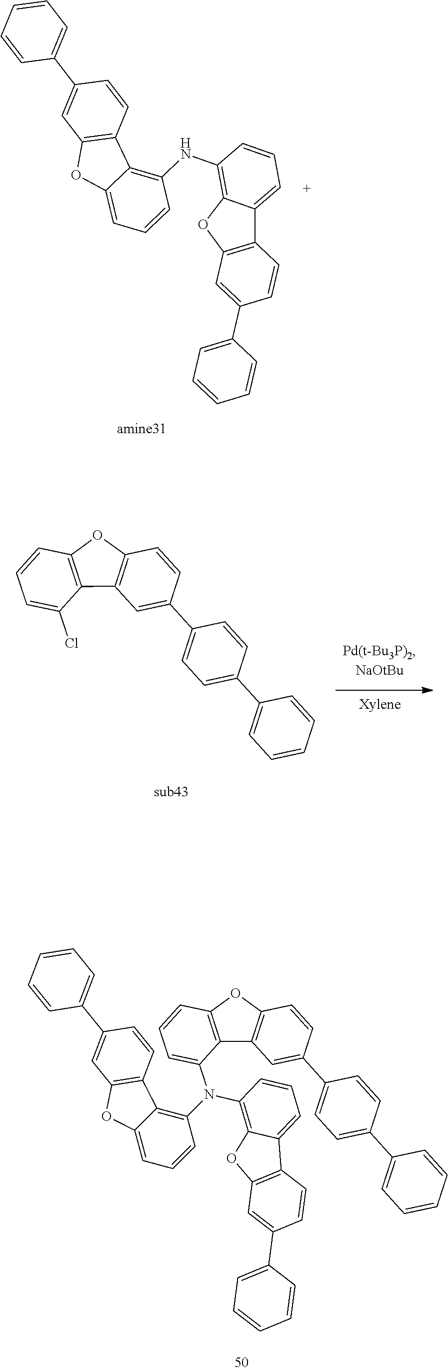 [SMILES: c1ccc(-c2ccc3c(c2)oc2c(Nc4cccc5oc6cc(-c7ccccc7)ccc6c45)cccc23)cc1 ; Clc1cccc2oc3ccc(-c4ccc(-c5ccccc5)cc4)cc3c12 ; c1ccc(-c2ccc(-c3ccc4oc5cccc(N(c6cccc7c6oc6cc(-c8ccccc8)ccc67)c6cccc7oc8cc(-c9ccccc9)ccc8c67)c5c4c3)cc2)cc1]

amine31 (15 g, 29.9 mmol), sub43 (11.1 g, 31.4 mmol) and sodium tert-butoxide (4.3 g, 44.9 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 13.2 g of Compound 50. (Yield: 54%, MS: [M+H]+=820)

### Synthesis Example 51

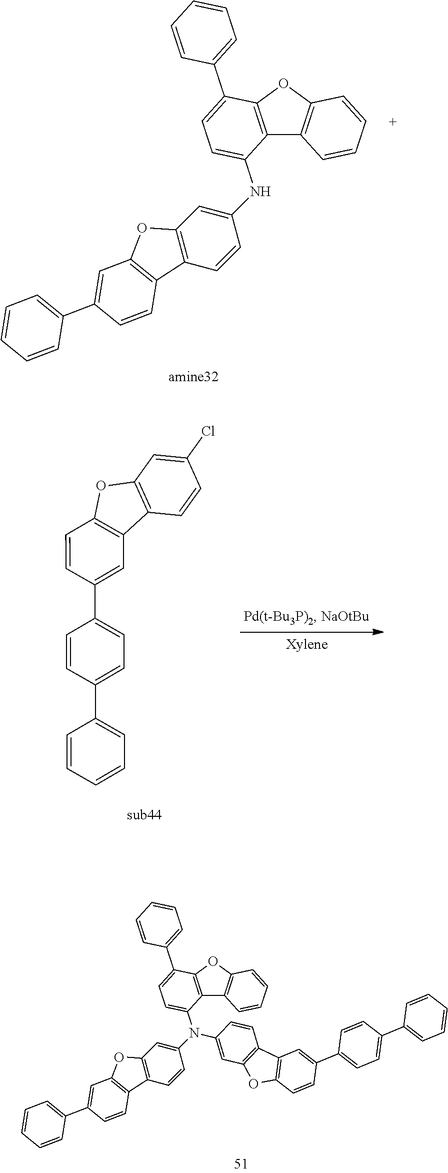 [SMILES: c1ccc(-c2ccc3c(c2)oc2cc(Nc4ccc(-c5ccccc5)c5oc6ccccc6c45)ccc23)cc1 ; Clc1ccc2c(c1)oc1ccc(-c3ccc(-c4ccccc4)cc3)cc12 ; c1ccc(-c2ccc(-c3ccc4oc5cc(N(c6ccc7c(c6)oc6cc(-c8ccccc8)ccc67)c6ccc(-c7ccccc7)c7oc8ccccc8c67)ccc5c4c3)cc2)cc1]

amine32 (15 g, 29.9 mmol), sub44 (11.1 g, 31.4 mmol) and sodium tert-butoxide (4.3 g, 44.9 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 16.4 g of Compound 51. (Yield: 67%, MS: [M+H]+=820)

### Synthesis Example 52

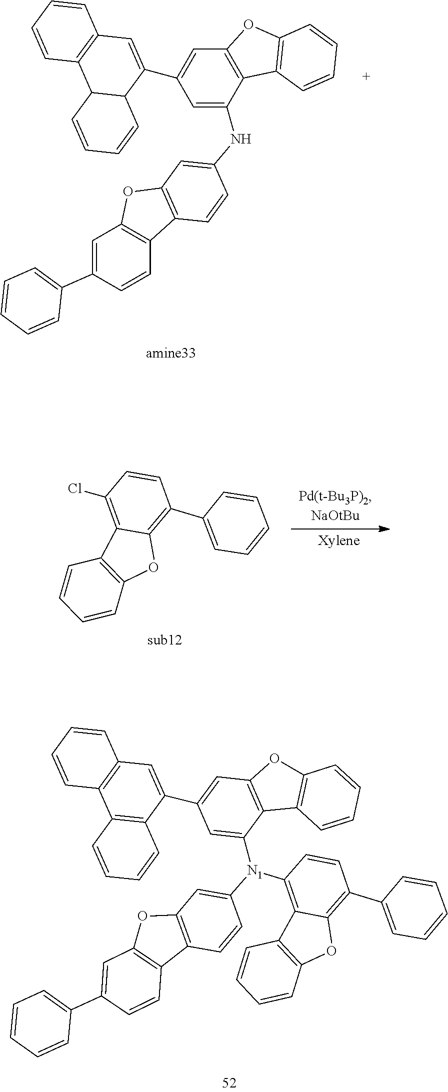

amine33 (15 g, 24.9 mmol), sub12 (7.3 g, 26.2 mmol) and sodium tert-butoxide (3.6 g, 37.4 mmol) were added to 300 ml of xylene under a nitrogen atmosphere, and mixture was stirred and refluxed. Then, bis(tri-tert-butylphosphine)palladium(0) (0.1 g, 0.2 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 12 g of Compound 52. (Yield: 57%, MS: [M+H]+=844)

### Synthesis Example 53

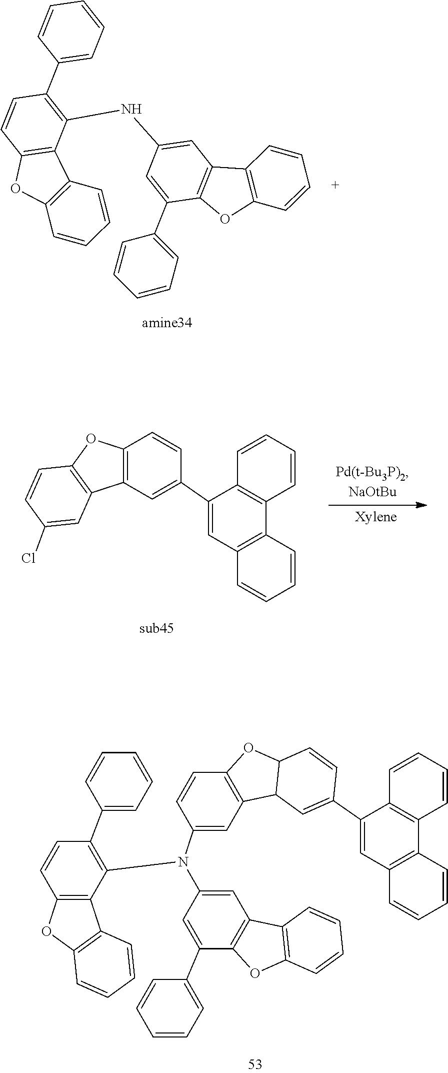 [SMILES: c1ccc(-c2ccc3oc4ccccc4c3c2Nc2cc(-c3ccccc3)c3oc4ccccc4c3c2)cc1 ; Clc1ccc2oc3ccc(-c4cc5ccccc5c5ccccc45)cc3c2c1 ; C1=CC2Oc3ccc(N(c4cc(-c5ccccc5)c5oc6ccccc6c5c4)c4c(-c5ccccc5)ccc5oc6ccccc6c45)cc3C2C=C1c1cc2ccccc2c2ccccc12]

amine34 (15 g, 29.9 mmol), sub45 (11.9 g, 31.4 mmol) and sodium tert-butoxide (4.3 g, 44.9 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 12.6 g of Compound 53. (Yield: 50%, MS: [M+H]+=844)

### Synthesis Example 54

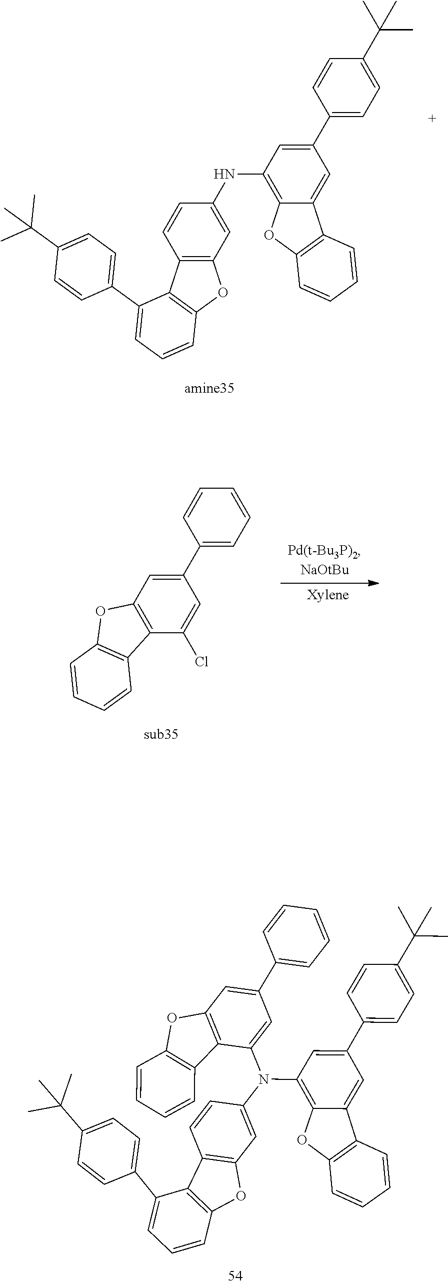

amine35 (15 g, 24.4 mmol), sub35 (7.2 g, 25.7 mmol) and sodium tert-butoxide (3.5 g, 36.7 mmol) were added to 300 ml of xylene under a nitrogen atmosphere, and mixture was stirred and refluxed. Then, bis(tri-tert-butylphosphine)palladium(0) (0.1 g, 0.2 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 13.6 g of Compound 54. (Yield: 65%, MS: [M+H]+=856)

### Synthesis Example 55

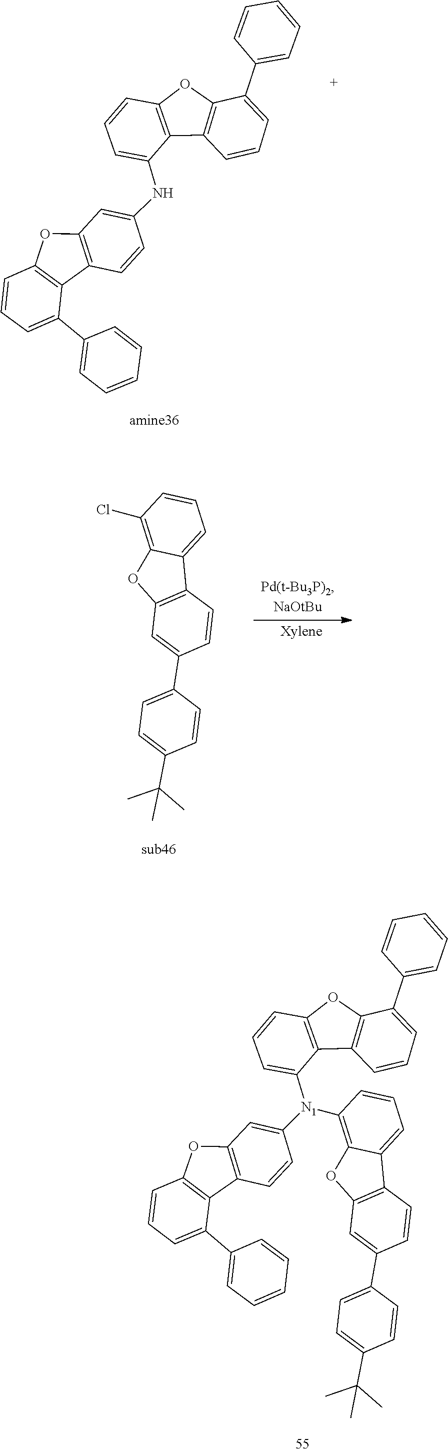

amine36 (15 g, 29.9 mmol), sub46 (10.5 g, 31.4 mmol) and sodium tert-butoxide (4.3 g, 44.9 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.6 g of Compound 55. (Yield: 61%, MS: [M+H]+=800)

### Synthesis Example 56

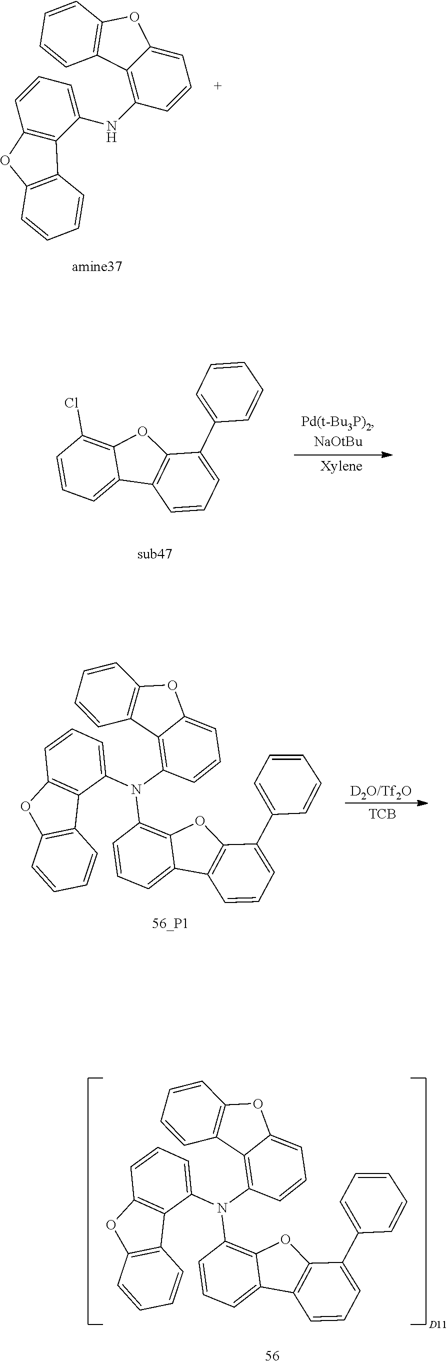

amine37 (15 g, 42.9 mmol), sub47 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 13.2 g of Compound 56_P1. (Yield: 52%, MS: [M+H]+=592)

Compound 56_P1 (10 g, 16.9 mmol) was added to 200 ml of 1,2,4-trichlorobenzene, and stirred at room temperature. In another vessel, deuterium oxide (8.5 g, 422.9 mmol) was added to trifluoromethanesulfonic anhydride (23.9 g, 84.6 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.4 g of Compound 56. (Yield: 63%, MS: [M+H]+=603).

### Synthesis Example 57

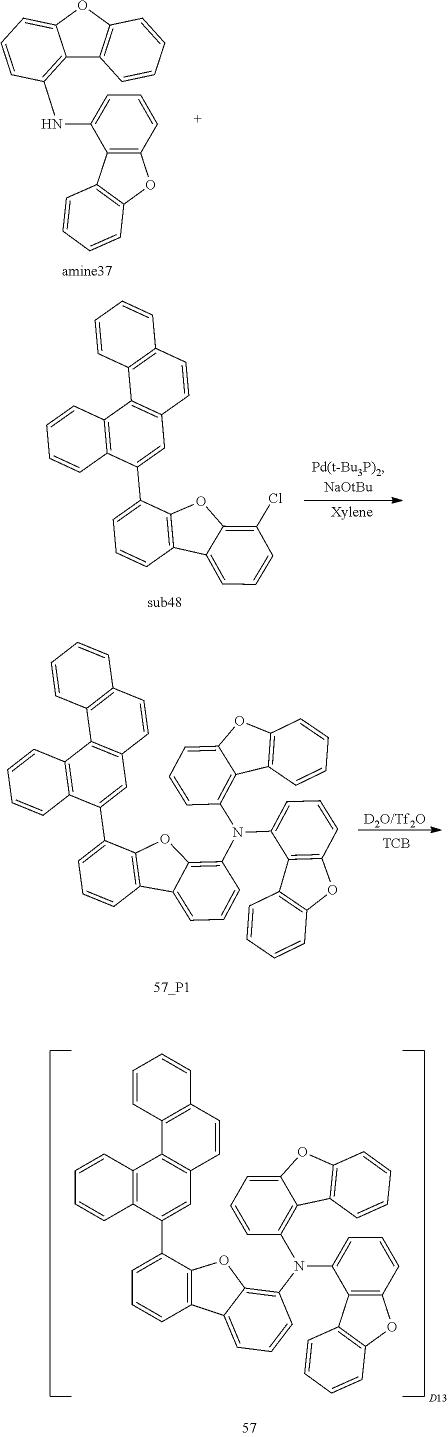

amine37 (15 g, 42.9 mmol), sub48 (19.3 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 21.3 g of Compound 57_P1. (Yield: 67%, MS: [M+H]+=742)

Compound 57_P1 (10 g, 13.5 mmol) was added to 200 ml of 1,2,4-trichlorobenzene, and stirred at room temperature. In another vessel, deuterium oxide (6.8 g, 337.3 mmol) was added to trifluoromethanesulfonic anhydride (19 g, 67.5 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 7 g of Compound 57. (Yield: 69%, MS: [M+H]+=755).

### Synthesis Example 58

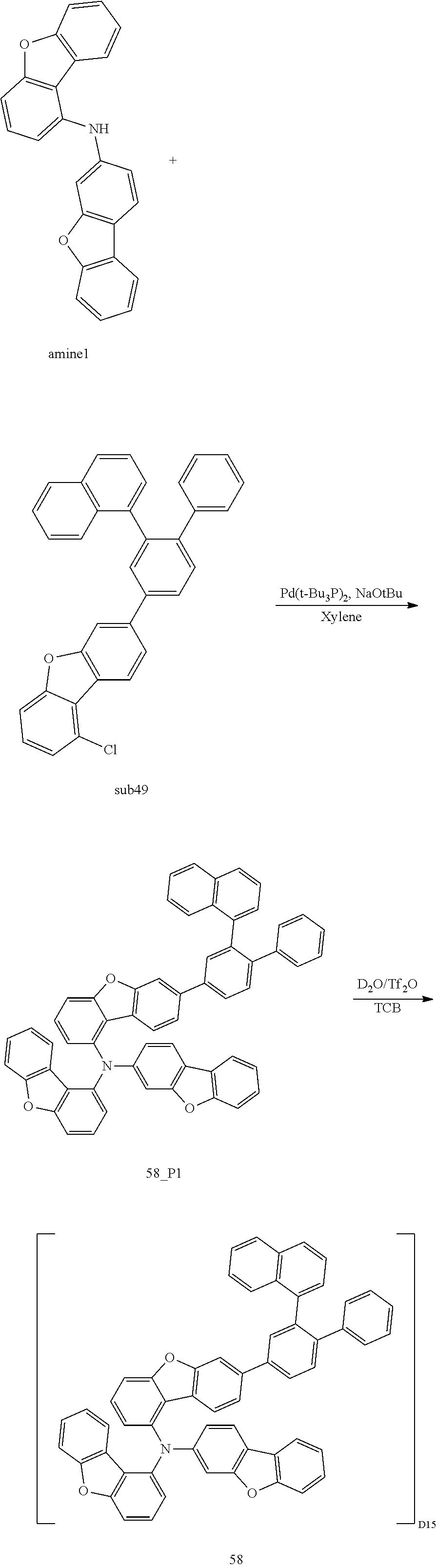

amine1 (15 g, 42.9 mmol), sub49 (21.7 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 17.7 g of Compound 58_P1. (Yield: 52%, MS: [M+H]+=794)

Compound 58_P1 (10 g, 12.6 mmol) was added to 200 ml of 1,2,4-trichlorobenzene, and stirred at room temperature. In another vessel, deuterium oxide (6.3 g, 315.2 mmol) was added to trifluoromethanesulfonic anhydride (17.8 g, 63 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.6 g of Compound 58. (Yield: 55%, MS: [M+H]+=809).

### Synthesis Example 59

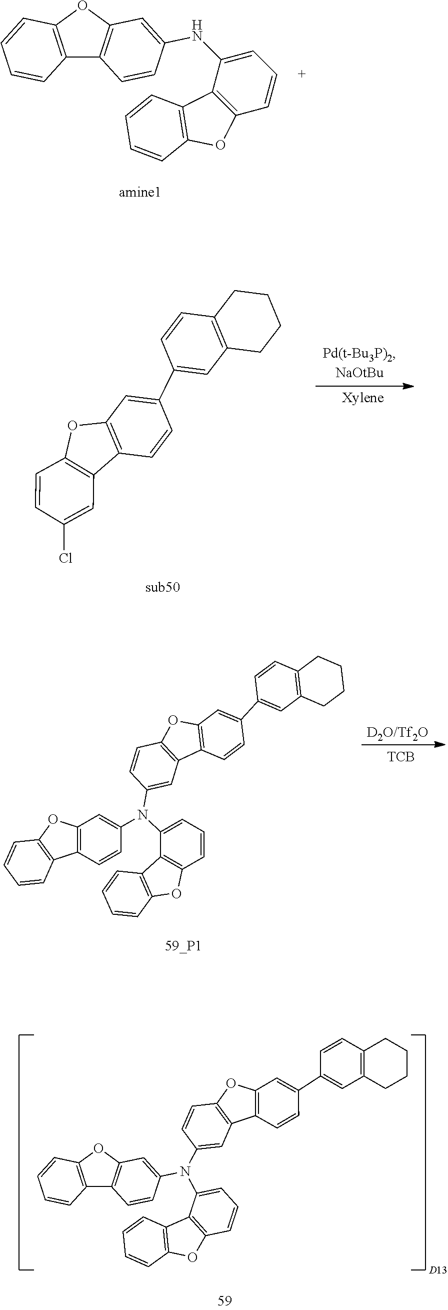

amine1 (15 g, 42.9 mmol), sub50 (15 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 16.9 g of Compound 59_P1. (Yield: 61%, MS: [M+H]+=646)

Compound 59_P1 (10 g, 15.5 mmol) was added to 200 ml of 1,2,4-trichlorobenzene, and stirred at room temperature. In another vessel, deuterium oxide (7.8 g, 387.5 mmol) was added to trifluoromethanesulfonic anhydride (21.9 g, 77.5 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.1 g of Compound 59. (Yield: 50%, MS: [M+H]+=659).

### Synthesis Example 60

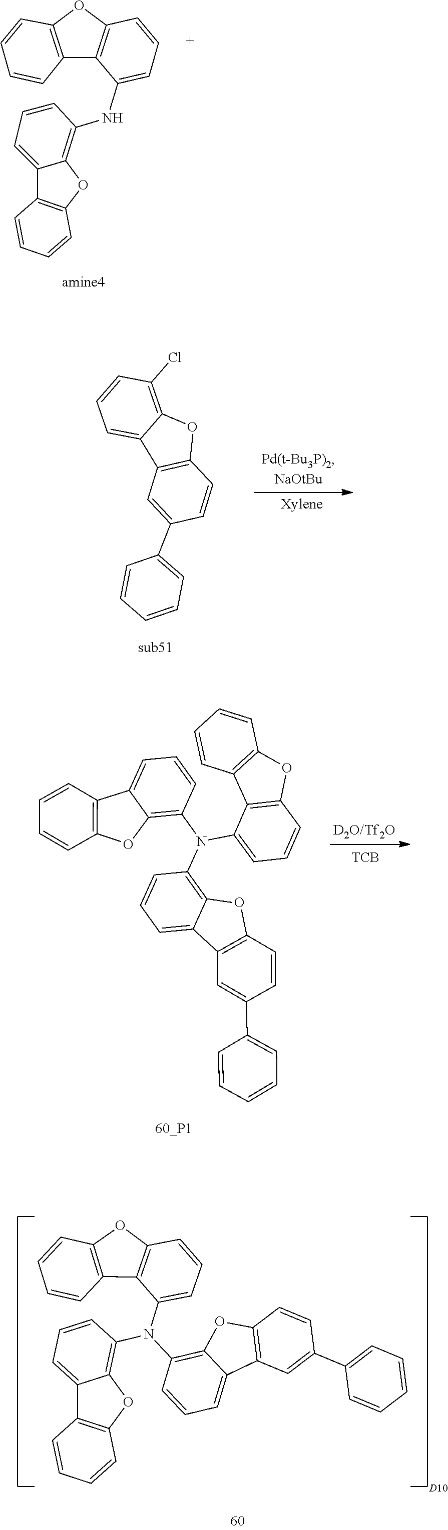

amine4 (15 g, 42.9 mmol), sub51 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15 g of Compound 60_P1. (Yield: 59%, MS: [M+H]+=592)

Compound 60_P1 (10 g, 16.9 mmol) was added to 200 ml of 1,2,4-trichlorobenzene, and stirred at room temperature. In another vessel, deuterium oxide (8.5 g, 422.9 mmol) was added to trifluoromethanesulfonic anhydride (23.9 g, 84.6 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.7 g of Compound 60. (Yield: 66%, MS: [M+H]+=602).

### Synthesis Example 61

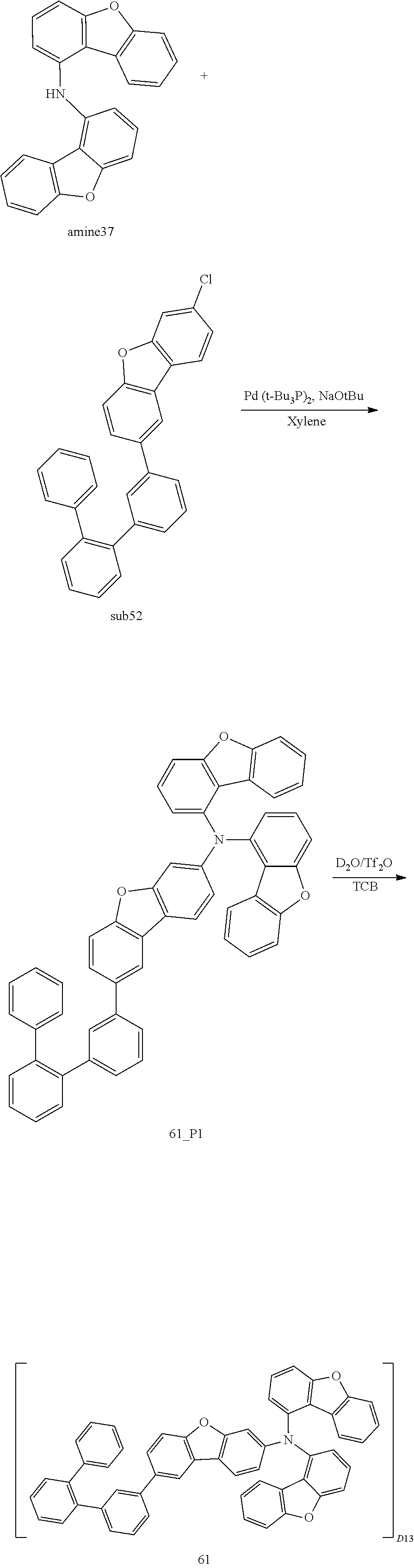

amine37 (15 g, 42.9 mmol), sub52 (19.4 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 22 g of Compound 61_P1. (Yield: 69%, MS: [M+H]+=744)

Compound 61_P1 (10 g, 13.5 mmol) was added to 200 ml of 1,2,4-trichlorobenzene, and stirred at room temperature. In another vessel, deuterium oxide (6.7 g, 336.4 mmol) was added to trifluoromethanesulfonic anhydride (19 g, 67.3 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 7.1 g of Compound 61. (Yield: 70%, MS: [M+H]+=757).

### Synthesis Example 62

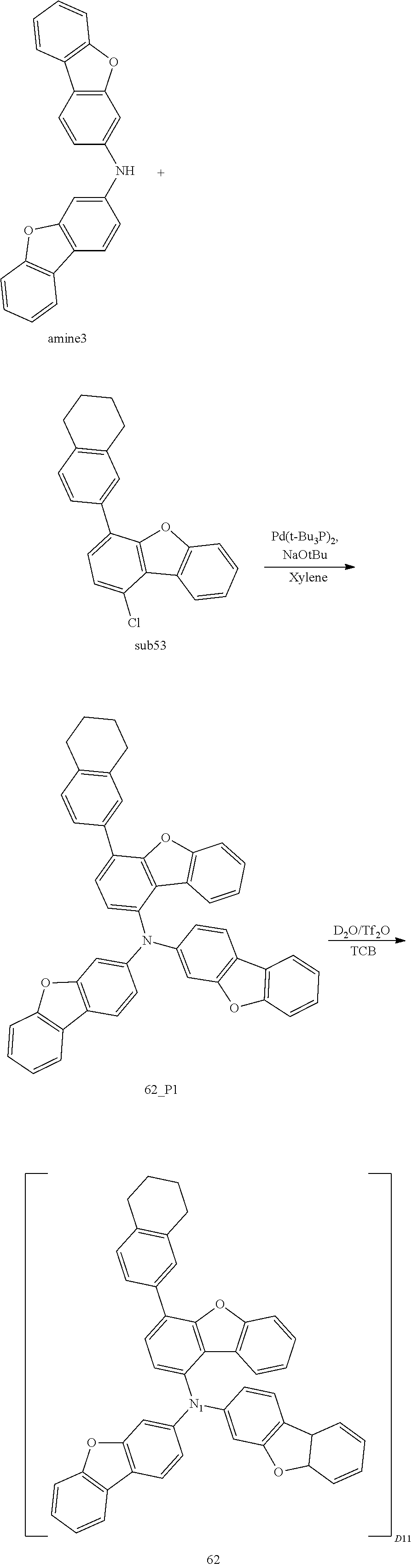

amine3 (15 g, 42.9 mmol), sub53 (15 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15 g of Compound 62_P1. (Yield: 54%, MS: [M+H]+=646)

Compound 62_P1 (10 g, 15.5 mmol) was added to 200 ml of 1,2,4-trichlorobenzene, and stirred at room temperature. In another vessel, deuterium oxide (7.8 g, 387.5 mmol) was added to trifluoromethanesulfonic anhydride (21.9 g, 77.5 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.4 g of Compound 62. (Yield: 63%, MS: [M+H]+=657).

### Synthesis Example 63

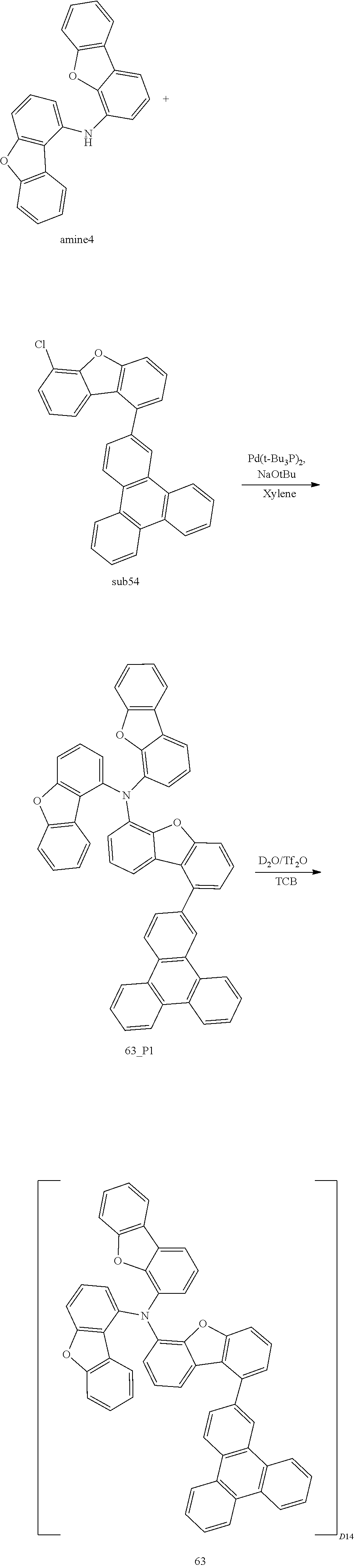

amine4 (15 g, 42.9 mmol), sub54 (19.3 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 17.8 g of Compound 63_P1. (Yield: 56%, MS: [M+H]+=742)

Compound 63_P1 (10 g, 13.5 mmol) was added to 200 ml of 1,2,4-trichlorobenzene, and stirred at room temperature. In another vessel, deuterium oxide (6.8 g, 337.3 mmol) was added to trifluoromethanesulfonic anhydride (19 g, 67.5 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.9 g of Compound 63. (Yield: 68%, MS: [M+H]+=756).

### Synthesis Example 64

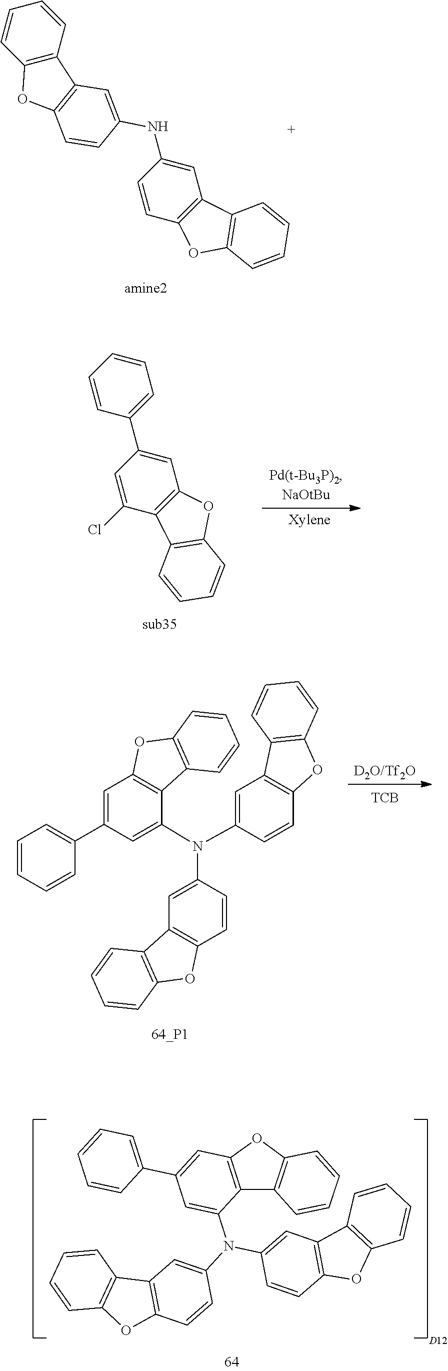

amine2 (15 g, 42.9 mmol), sub35 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 16.5 g of Compound 64_P1. (Yield: 65%, MS: [M+H]+=592)

Compound 64_P1 (10 g, 16.9 mmol) was added to 200 ml of 1,2,4-trichlorobenzene, and stirred at room temperature. In another vessel, deuterium oxide (8.5 g, 422.9 mmol) was added to trifluoromethanesulfonic anhydride (23.9 g, 84.6 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.4 g of Compound 64. (Yield: 63%, MS: [M+H]+=604).

### Synthesis Example 65

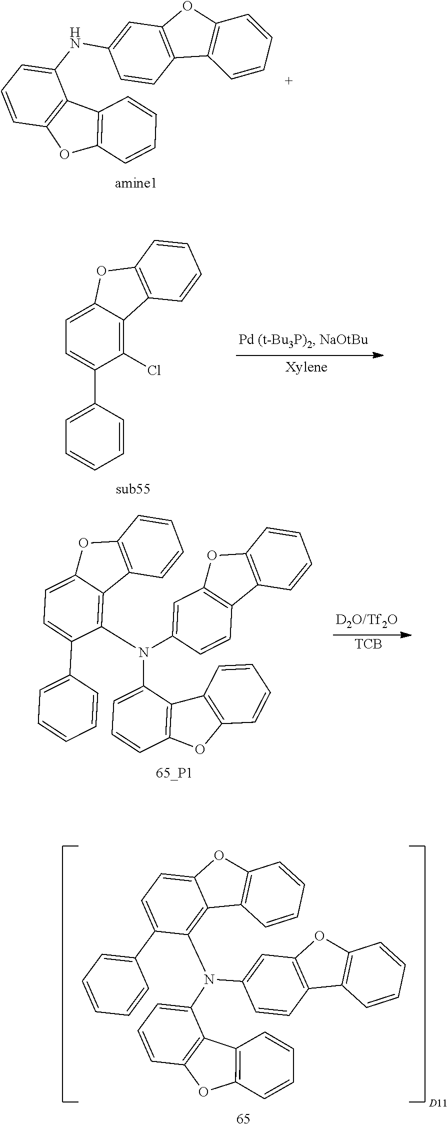

amine1 (15 g, 42.9 mmol), sub55 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.5 g of Compound 65_P1. (Yield: 57%, MS: [M+H]+=592)

Compound 65_P1 (10 g, 16.9 mmol) was added to 200 ml of 1,2,4-trichlorobenzene, and stirred at room temperature. In another vessel, deuterium oxide (8.5 g, 422.9 mmol) was added to trifluoromethanesulfonic anhydride (23.9 g, 84.6 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6 g of Compound 65. (Yield: 59%, MS: [M+H]+=603).

### Synthesis Example 66

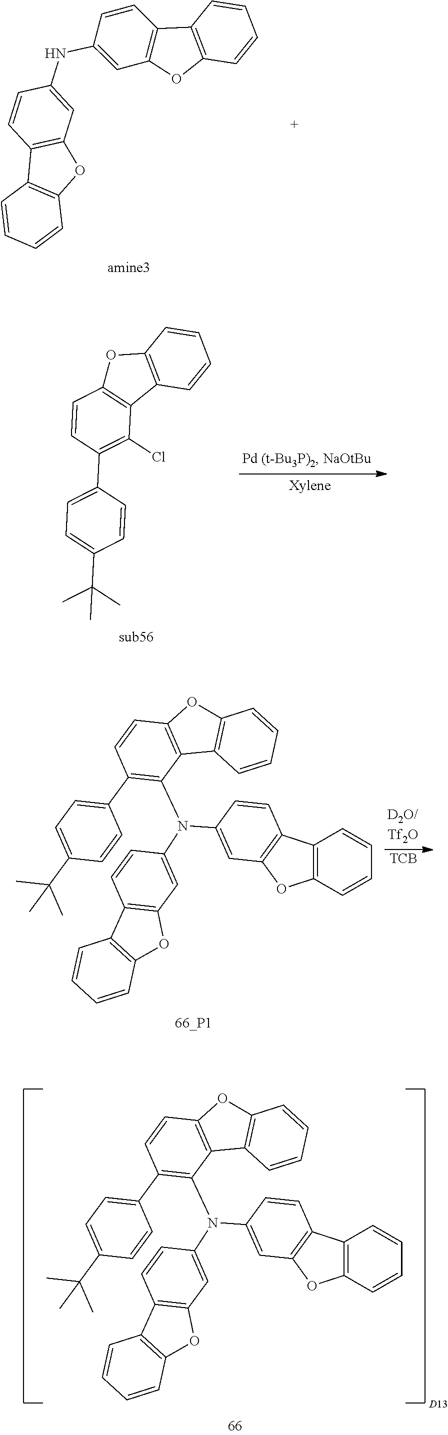

amine3 (15 g, 42.9 mmol), sub56 (15.1 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15.8 g of Compound 66_P1. (Yield: 57%, MS: [M+H]+=648)

Compound 66_P1 (10 g, 15.4 mmol) was added to 200 ml of 1,2,4-trichlorobenzene, and stirred at room temperature. In another vessel, deuterium oxide (7.7 g, 386.2 mmol) was added to trifluoromethanesulfonic anhydride (21.8 g, 77.2 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.5 g of Compound 66. (Yield: 54%, MS: [M+H]+=661).

### Synthesis Example 67

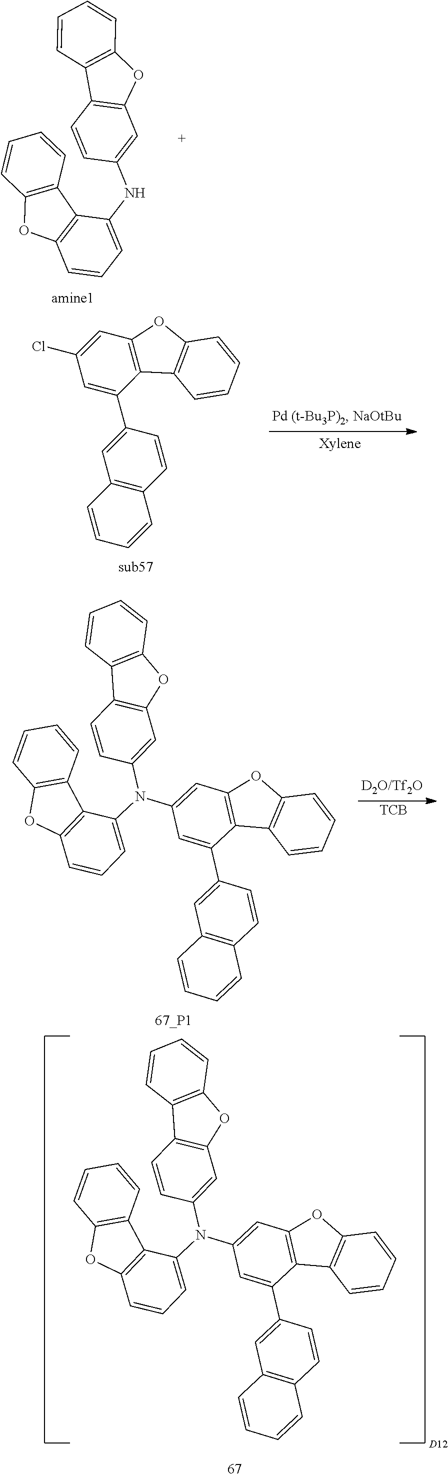

amine1 (15 g, 42.9 mmol), sub57 (14.8 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15.4 g of Compound 67_P1. (Yield: 56%, MS: [M+H]+=642)

Compound 67_P1 (10 g, 15.6 mmol) was added to 200 ml of 1,2,4-trichlorobenzene, and stirred at room temperature. In another vessel, deuterium oxide (7.8 g, 389.9 mmol) was added to trifluoromethanesulfonic anhydride (22 g, 78 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.4 g of Compound 67. (Yield: 63%, MS: [M+H]+=654).

### Synthesis Example 68

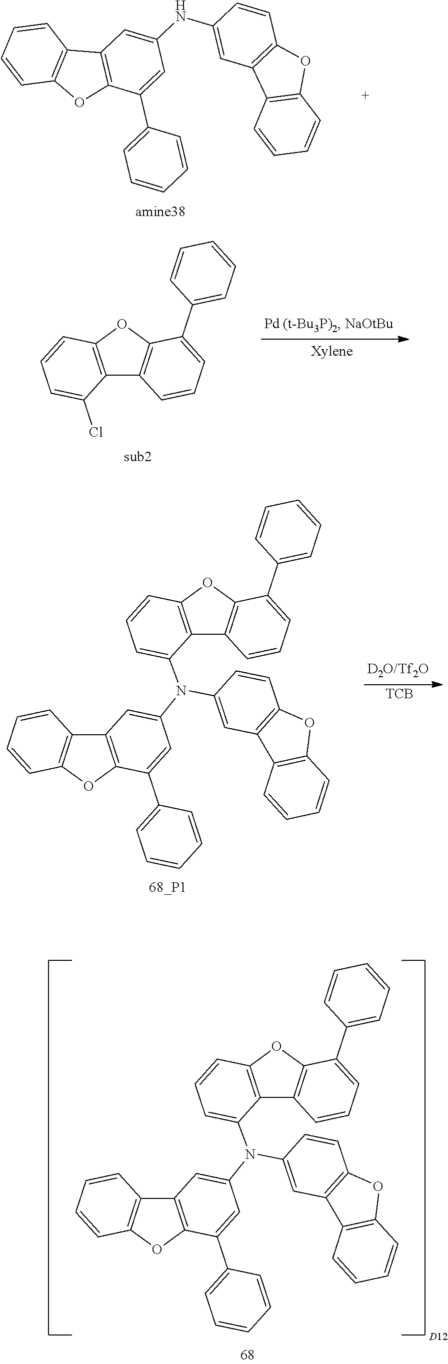

amine38 (15 g, 35.3 mmol), sub2 (10.3 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 16.2 g of Compound 68_P1. (Yield: 69%, MS: [M+H]+=668)

Compound 68_P1 (10 g, 15 mmol) was added to 200 ml of 1,2,4-trichlorobenzene, and stirred at room temperature. In another vessel, deuterium oxide (7.5 g, 374.7 mmol) was added to trifluoromethanesulfonic anhydride (21.1 g, 74.9 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6 g of Compound 68. (Yield: 59%, MS: [M+H]+=680).

### Synthesis Example 69

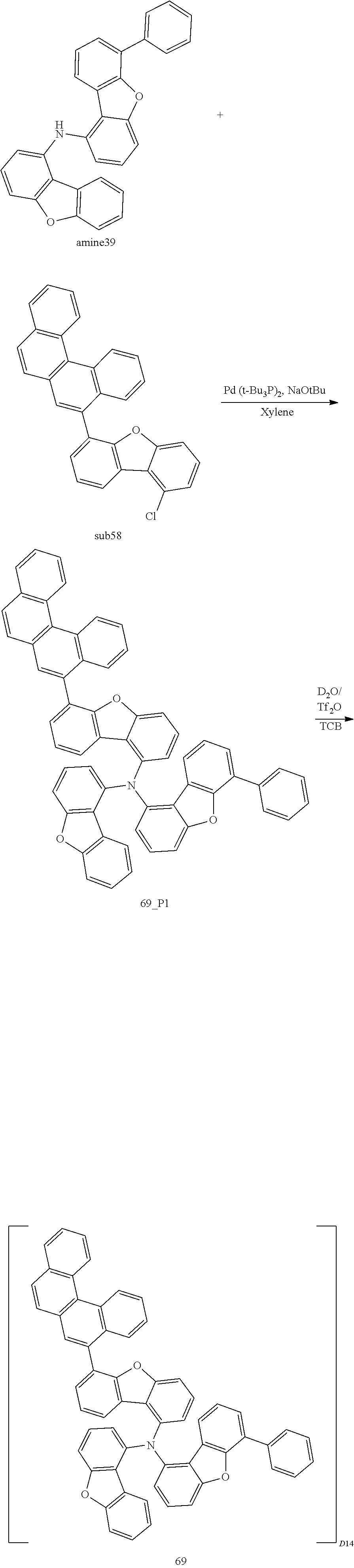

amine39 (15 g, 35.3 mmol), sub58 (15.9 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 18.2 g of Compound 69_P1. (Yield: 63%, MS: [M+H]+=818)

Compound 69_P1 (10 g, 12.2 mmol) was added to 200 ml of 1,2,4-trichlorobenzene, and stirred at room temperature. In another vessel, deuterium oxide (6.1 g, 305.9 mmol) was added to trifluoromethanesulfonic anhydride (17.3 g, 61.2 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.7 g of Compound 69. (Yield: 56%, MS: [M+H]+=832).

### Synthesis Example 70

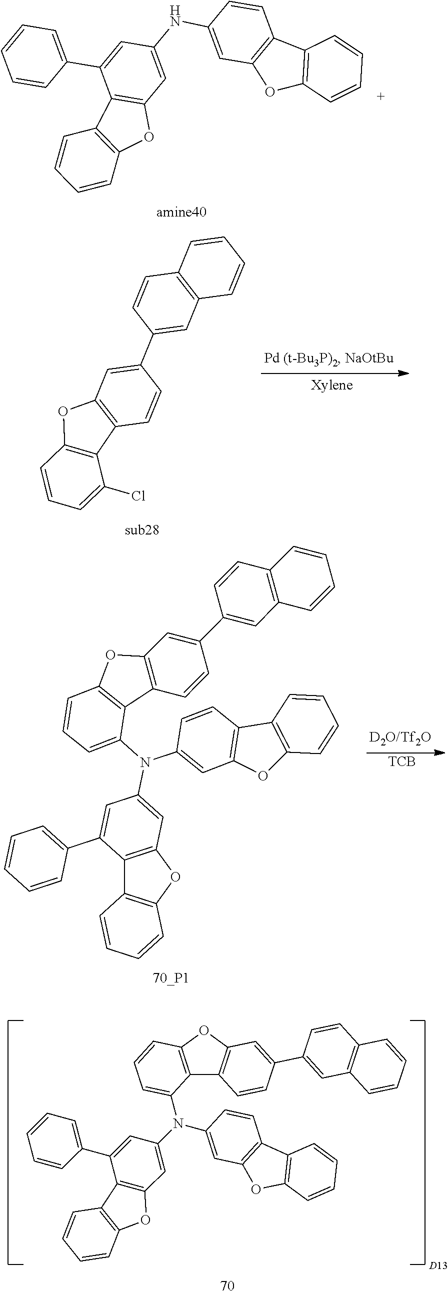

amine40 (15 g, 35.3 mmol), sub28 (12.2 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.4 g of Compound 70_P1. (Yield: 57%, MS: [M+H]+=718)

Compound 70_P1 (10 g, 13.9 mmol) was added to 200 ml of 1,2,4-trichlorobenzene, and stirred at room temperature. In another vessel, deuterium oxide (7 g, 348.6 mmol) was added to trifluoromethanesulfonic anhydride (19.7 g, 69.7 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.4 g of Compound 70. (Yield: 63%, MS: [M+H]+=731).

### Synthesis Example 71

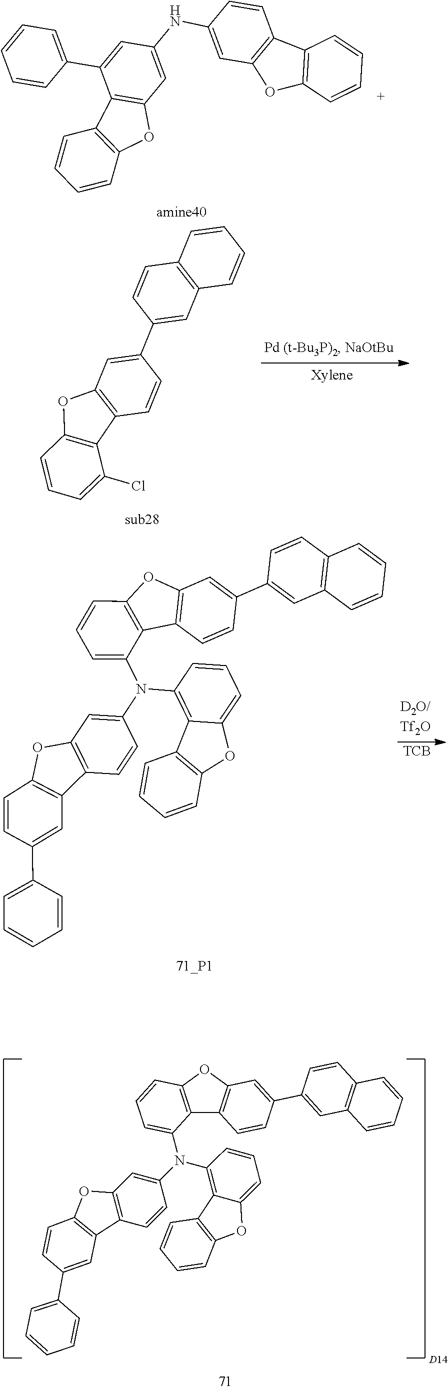

amine41 (15 g, 35.3 mmol), sub28 (12.2 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 12.6 g of Compound 71_P1. (Yield: 50%, MS: [M+H]+=718)

Compound 71_P1 (10 g, 13.9 mmol) was added to 200 ml of 1,2,4-trichlorobenzene, and stirred at room temperature. In another vessel, deuterium oxide (7 g, 348.6 mmol) was added to trifluoromethanesulfonic anhydride (19.7 g, 69.7 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.7 g of Compound 71. (Yield: 66%, MS: [M+H]+=732).

### Synthesis Example 72

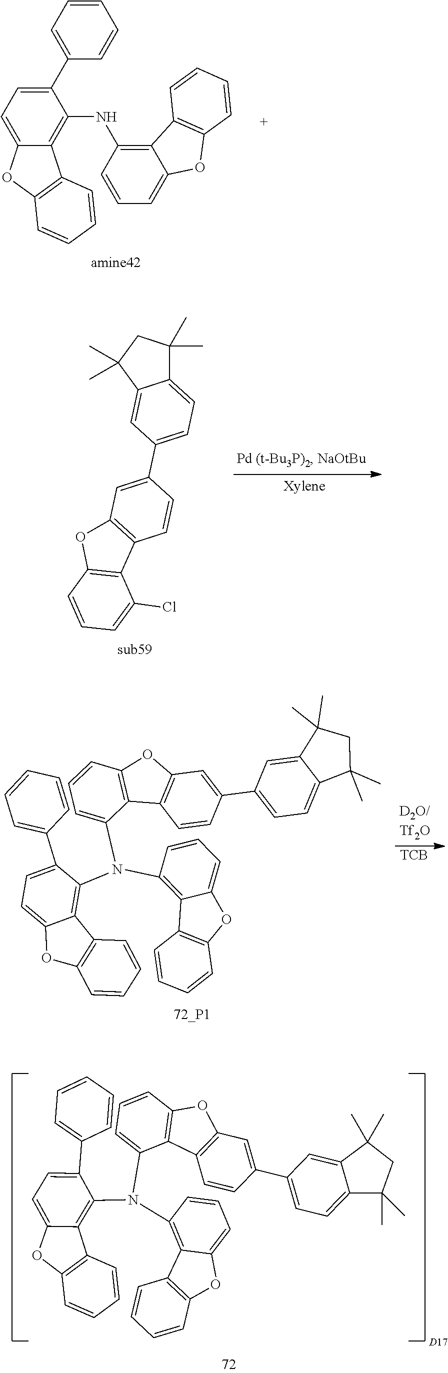

amine42 (15 g, 35.3 mmol), sub59 (13.9 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 16.1 g of Compound 72_P1. (Yield: 60%, MS: [M+H]+=764)

Compound 72_P1 (10 g, 13.1 mmol) was added to 200 ml of 1,2,4-trichlorobenzene, and stirred at room temperature. In another vessel, deuterium oxide (6.6 g, 327.5 mmol) was added to trifluoromethanesulfonic anhydride (18.5 g, 65.5 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.2 g of Compound 72. (Yield: 51%, MS: [M+H]+=781).

### Synthesis Example 73

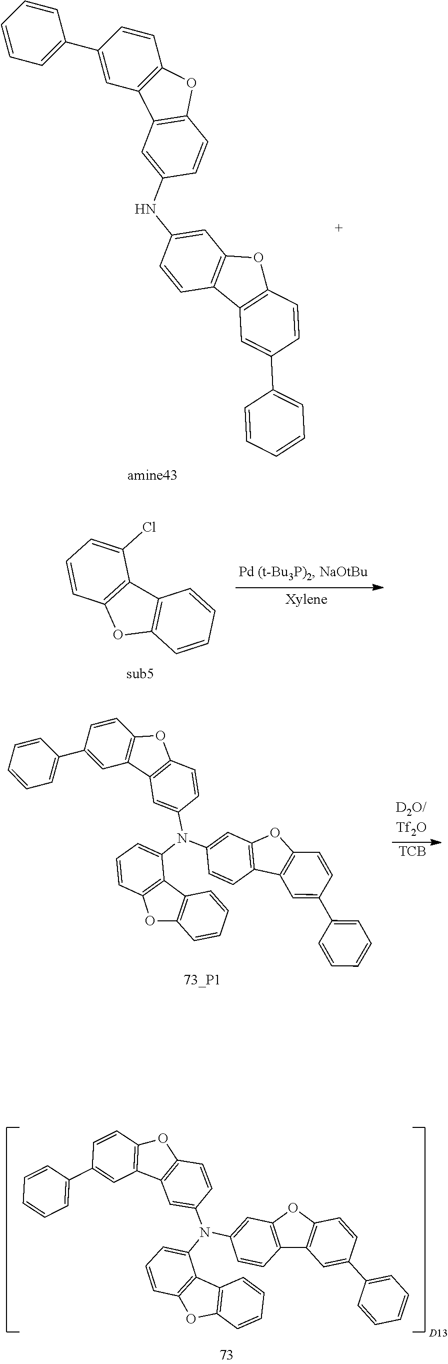

amine43 (15 g, 29.9 mmol), sub5 (6.4 g, 31.4 mmol) and sodium tert-butoxide (4.3 g, 44.9 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 10.4 g of Compound 73_P1. (Yield: 52%, MS: [M+H]+=668)

Compound 73_P1 (10 g, 15 mmol) was added to 200 ml of 1,2,4-trichlorobenzene, and stirred at room temperature. In another vessel, deuterium oxide (7.5 g, 374.7 mmol) was added to trifluoromethanesulfonic anhydride (21.1 g, 74.9 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6 g of Compound 73. (Yield: 59%, MS: [M+H]+=681).

### Synthesis Example 74

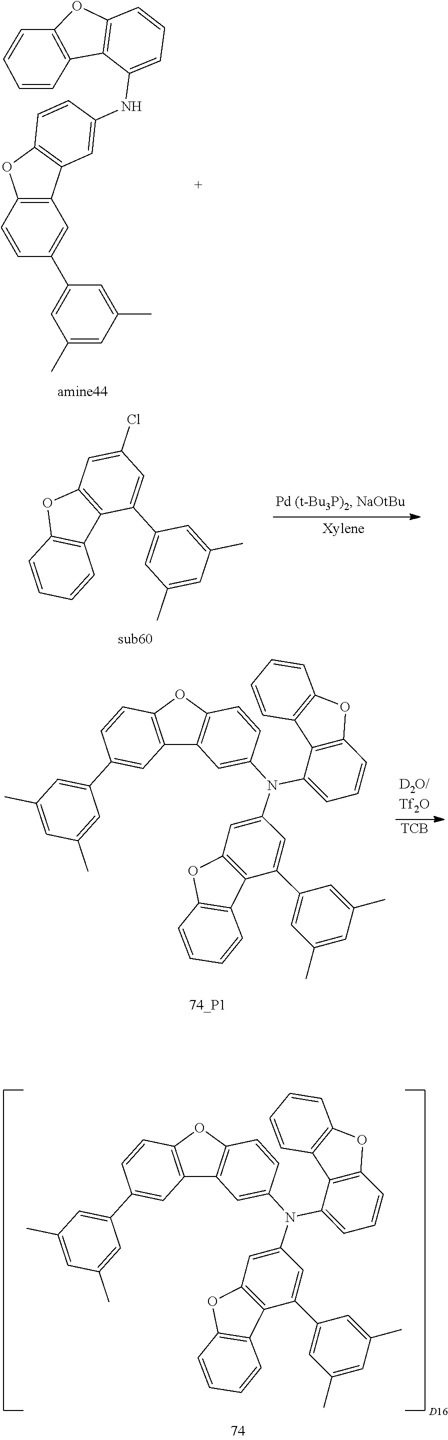

amine44 (15 g, 33.1 mmol), sub60 (10.7 g, 34.7 mmol) and sodium tert-butoxide (4.8 g, 49.6 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 12.7 g of Compound 74_P1. (Yield: 53%, MS: [M+H]+=724)

Compound 74_P1 (10 g, 13.8 mmol) was added to 200 ml of 1,2,4-trichlorobenzene, and stirred at room temperature. In another vessel, deuterium oxide (6.9 g, 345.6 mmol) was added to trifluoromethanesulfonic anhydride (19.5 g, 69.1 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.4 g of Compound 74. (Yield: 63%, MS: [M+H]+=740).

### Synthesis Example 75

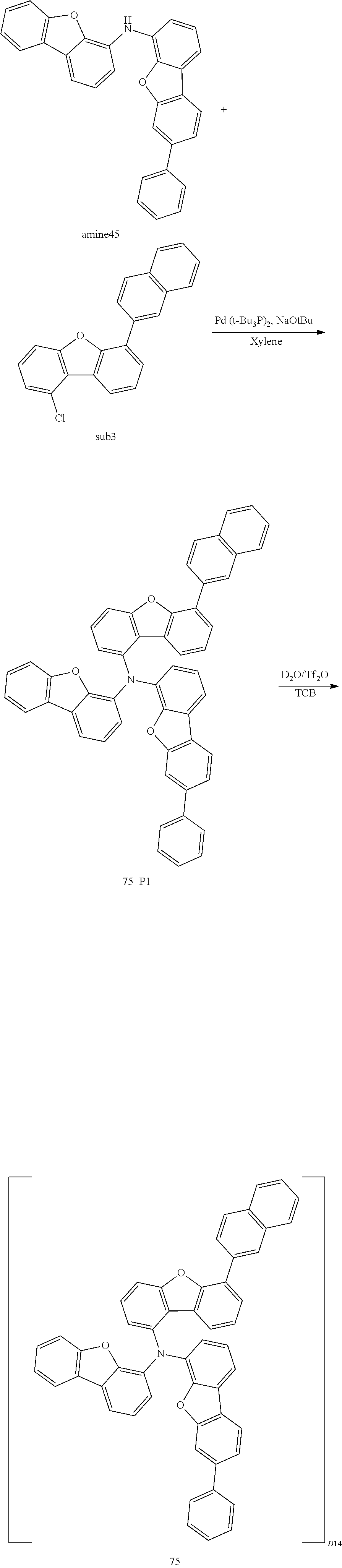

amine45 (15 g, 35.3 mmol), sub3 (12.2 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.2 g of Compound 75_P1. (Yield: 56%, MS: [M+H]+=718)

Compound 75_P1 (10 g, 13.9 mmol) was added to 200 ml of 1,2,4-trichlorobenzene, and stirred at room temperature. In another vessel, deuterium oxide (7 g, 348.6 mmol) was added to trifluoromethanesulfonic anhydride (19.7 g, 69.7 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.6 g of Compound 75. (Yield: 65%, MS: [M+H]+=732).

### Synthesis Example 76

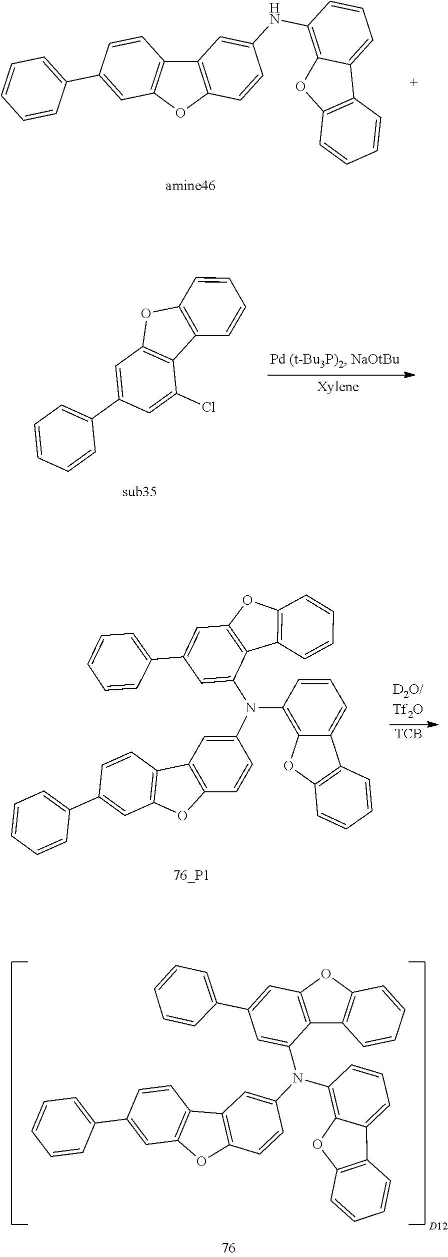

amine46 (15 g, 35.3 mmol), sub35 (10.3 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 12.7 g of Compound 76_P1. (Yield: 54%, MS: [M+H]+=668)

Compound 76_P1 (10 g, 15 mmol) was added to 200 ml of 1,2,4-trichlorobenzene, and stirred at room temperature. In another vessel, deuterium oxide (7.5 g, 374.7 mmol) was added to trifluoromethanesulfonic anhydride (21.1 g, 74.9 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.6 g of Compound 76. (Yield: 65%, MS: [M+H]+=680).

### Synthesis Example 77

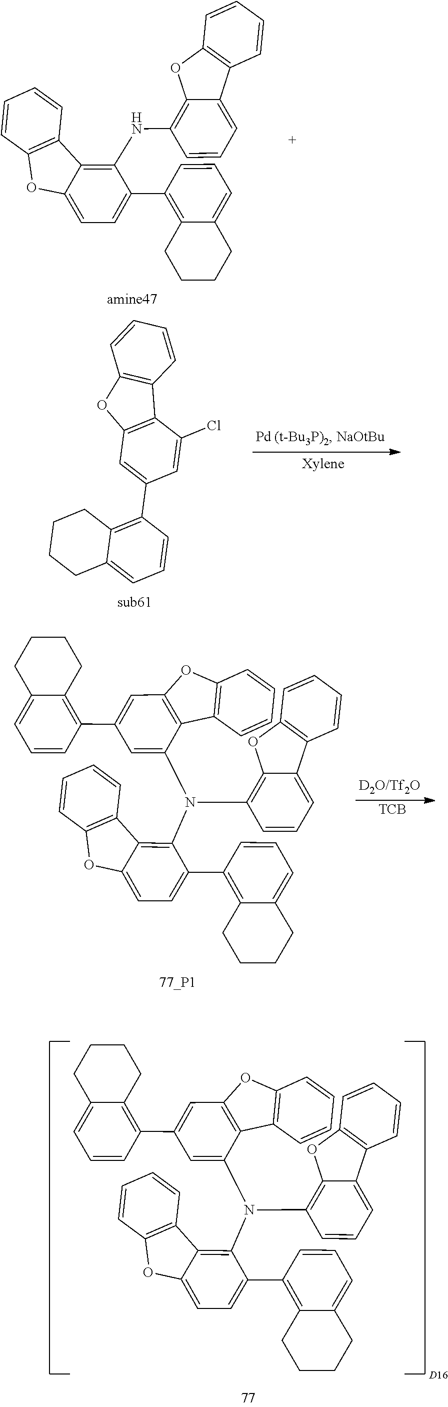

amine47 (15 g, 31.3 mmol), sub61 (10.9 g, 32.8 mmol) and sodium tert-butoxide (4.5 g, 46.9 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15.3 g of Compound 77_P1. (Yield: 63%, MS: [M+H]+=776)

Compound 77_P1 (10 g, 12.9 mmol) was added to 200 ml of 1,2,4-trichlorobenzene, and stirred at room temperature. In another vessel, deuterium oxide (6.5 g, 322.5 mmol) was added to trifluoromethanesulfonic anhydride (18.2 g, 64.5 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 7 g of Compound 77. (Yield: 69%, MS: [M+H]+=792).

### Synthesis Example 78

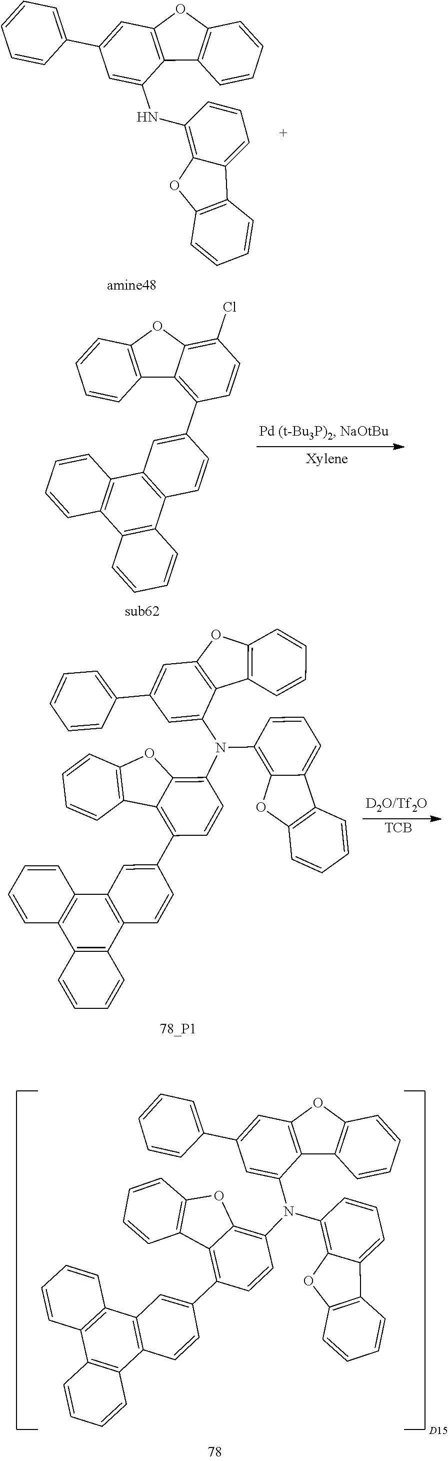

amine48 (15 g, 35.3 mmol), sub62 (15.9 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 16.4 g of Compound 78_P1. (Yield: 57%, MS: [M+H]+=818)

Compound 78_P1 (10 g, 12.2 mmol) was added to 200 ml of 1,2,4-trichlorobenzene, and stirred at room temperature. In another vessel, deuterium oxide (6.1 g, 305.9 mmol) was added to trifluoromethanesulfonic anhydride (17.3 g, 61.2 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.3 g of Compound 78. (Yield: 52%, MS: [M+H]+=833).

### Synthesis Example 79

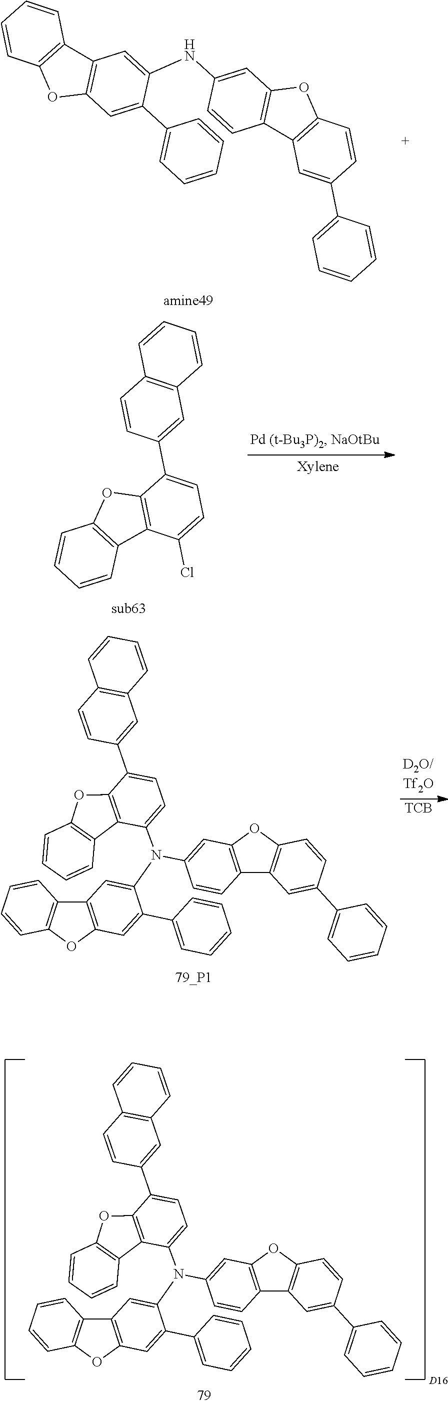

amine49 (15 g, 29.9 mmol), sub63 (10.3 g, 31.4 mmol) and sodium tert-butoxide (4.3 g, 44.9 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 13.8 g of Compound 79_P1. (Yield: 58%, MS: [M+H]+=794)

Compound 79_P1 (10 g, 12.6 mmol) was added to 200 ml of 1,2,4-trichlorobenzene, and stirred at room temperature. In another vessel, deuterium oxide (6.3 g, 315.2 mmol) was added to trifluoromethanesulfonic anhydride (17.8 g, 63 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.3 g of Compound 79. (Yield: 52%, MS: [M+H]+=810).

### Synthesis Example 80

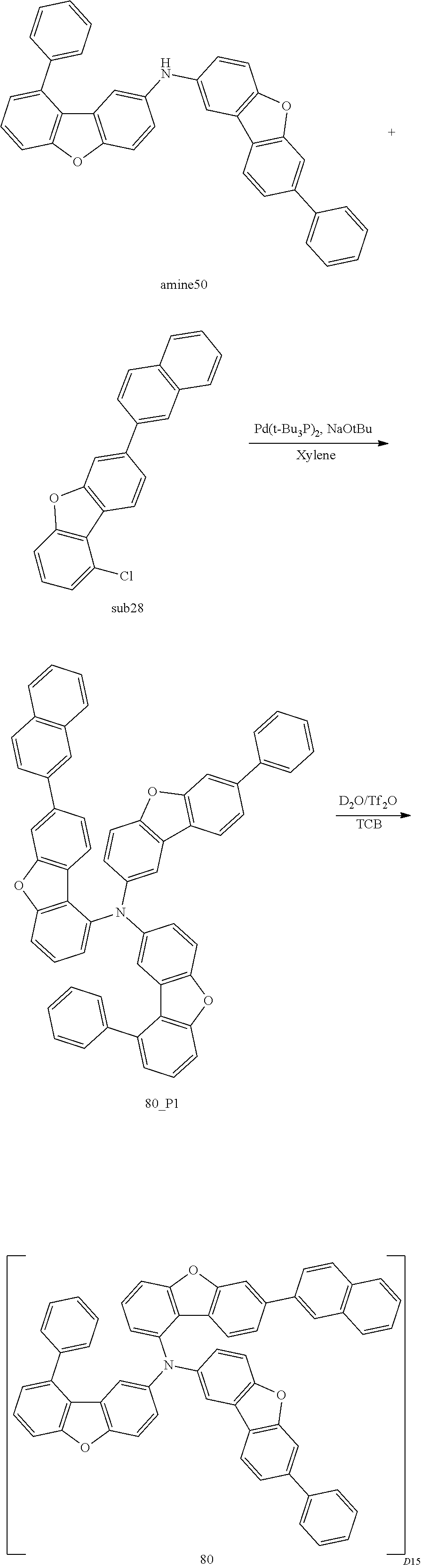

amine50 (15 g, 29.9 mmol), sub28 (10.3 g, 31.4 mmol) and sodium tert-butoxide (4.3 g, 44.9 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15.2 g of Compound 80_P1. (Yield: 64%, MS: [M+H]+=794)

Compound 80_P1 (10 g, 12.6 mmol) was added to 200 ml of 1,2,4-trichlorobenzene, and stirred at room temperature. In another vessel, deuterium oxide (6.3 g, 315.2 mmol) was added to trifluoromethanesulfonic anhydride (17.8 g, 63 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.8 g of Compound 80. (Yield: 57%, MS: [M+H]+=809).

### Synthesis Example 81

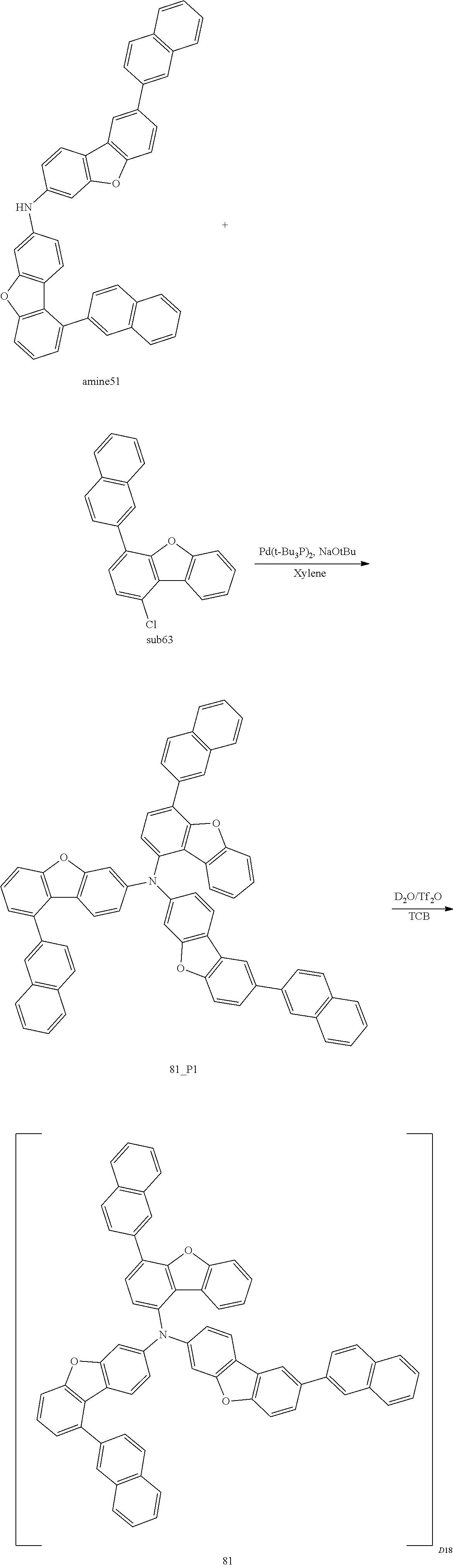

amine51 (15 g, 24.9 mmol), sub63 (8.6 g, 26.2 mmol) and sodium tert-butoxide (3.6 g, 37.4 mmol) were added to 300 ml of xylene under a nitrogen atmosphere, and mixture was stirred and refluxed. Then, bis(tri-tert-butylphosphine)palladium(0) (0.1 g, 0.2 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.7 g of Compound 81_P1. (Yield: 66%, MS: [M+H]+=894)

Compound 81_P1 (10 g, 11.2 mmol) was added to 200 ml of 1,2,4-trichlorobenzene, and stirred at room temperature. In another vessel, deuterium oxide (5.6 g, 279.9 mmol) was added to trifluoromethanesulfonic anhydride (15.8 g, 56 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.2 g of Compound 81. (Yield: 61%, MS: [M+H]+=912).

### Synthesis Example 82

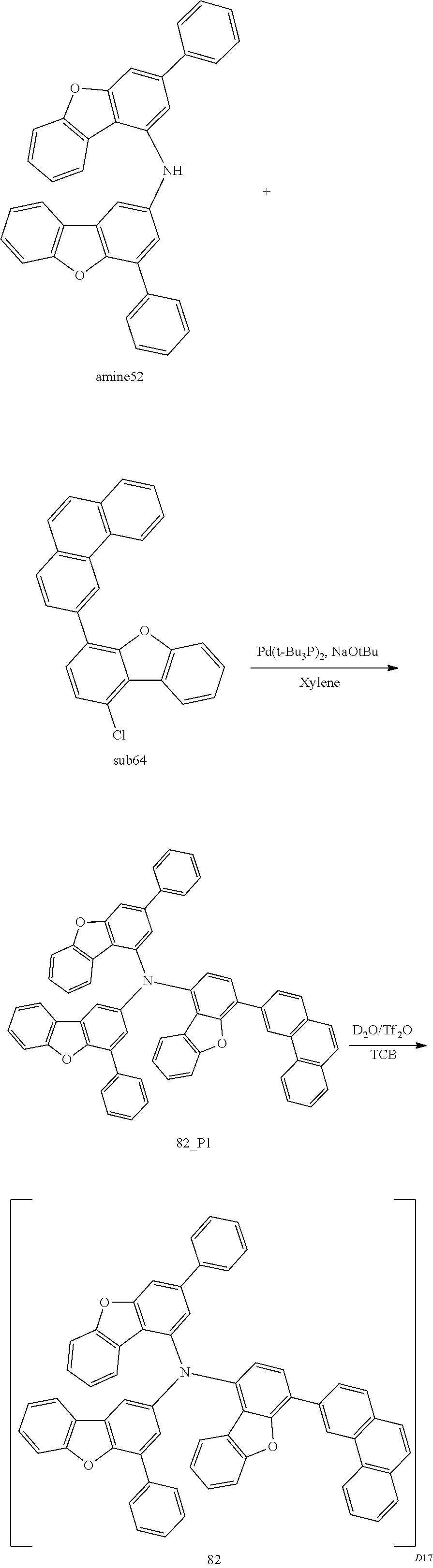

amine52 (15 g, 29.9 mmol), sub64 (11.9 g, 31.4 mmol) and sodium tert-butoxide (4.3 g, 44.9 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.6 g of Compound 82_P1. (Yield: 58%, MS: [M+H]+=844)

Compound 82_P1 (10 g, 11.9 mmol) was added to 200 ml of 1,2,4-trichlorobenzene, and stirred at room temperature. In another vessel, deuterium oxide (5.9 g, 296.5 mmol) was added to trifluoromethanesulfonic anhydride (16.7 g, 59.3 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.9 g of Compound 82. (Yield: 68%, MS: [M+H]+=861).

### Synthesis Example 83

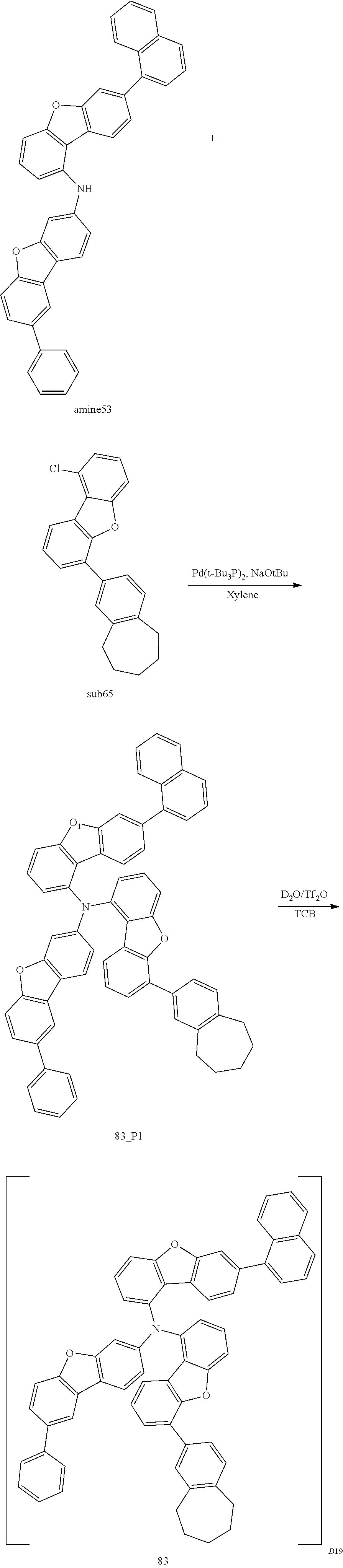

amine53 (15 g, 29.9 mmol), sub65 (10.9 g, 31.4 mmol) and sodium tert-butoxide (4.3 g, 44.9 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 18 g of Compound 83_P1. (Yield: 70%, MS: [M+H]+=862)

Compound 83_P1 (10 g, 11.6 mmol) was added to 200 ml of 1,2,4-trichlorobenzene, and stirred at room temperature. In another vessel, deuterium oxide (5.8 g, 290.3 mmol) was added to trifluoromethanesulfonic anhydride (16.4 g, 58.1 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.5 g of Compound 83. (Yield: 64%, MS: [M+H]+=881).

### Synthesis Example 84

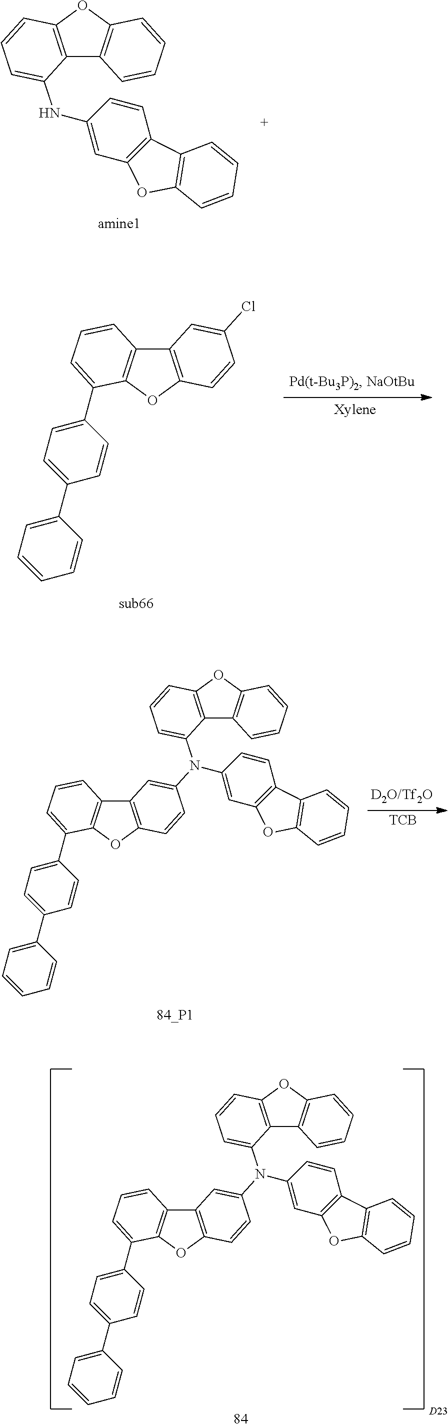

amine1 (15 g, 42.9 mmol), sub66 (16 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 16 g of Compound 84_P1. (Yield: 56%, MS: [M+H]+=668)

Compound 84_P1 (10 g, 15 mmol) was added to 200 ml of 1,2,4-trichlorobenzene, and stirred at room temperature. In another vessel, deuterium oxide (10.8 g, 539.6 mmol) was added to trifluoromethanesulfonic anhydride (38.1 g, 134.9 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 7.2 g of Compound 84. (Yield: 70%, MS: [M+H]+=691).

### Synthesis Example 85

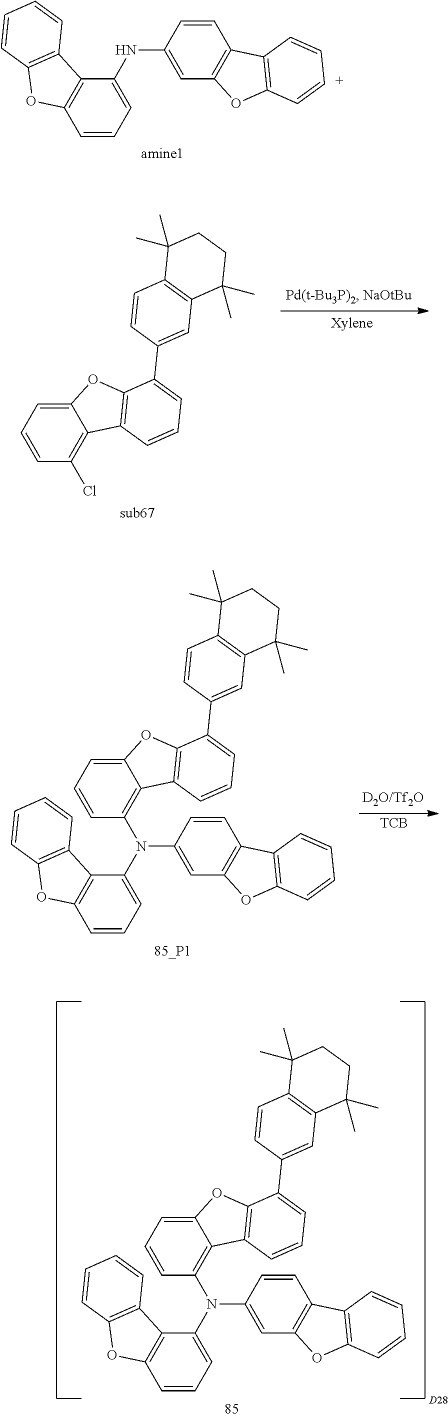

amine1 (15 g, 42.9 mmol), sub67 (17.5 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 16.6 g of Compound 85_P1. (Yield: 55%, MS: [M+H]+=702)

Compound 85_P1 (10 g, 14.3 mmol) was added to 200 ml of 1,2,4-trichlorobenzene, and stirred at room temperature. In another vessel, deuterium oxide (10.3 g, 513.3 mmol) was added to trifluoromethanesulfonic anhydride (36.2 g, 128.3 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 4 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 7.1 g of Compound 85. (Yield: 68%, MS: [M+H]+=730).

### Synthesis Example 86

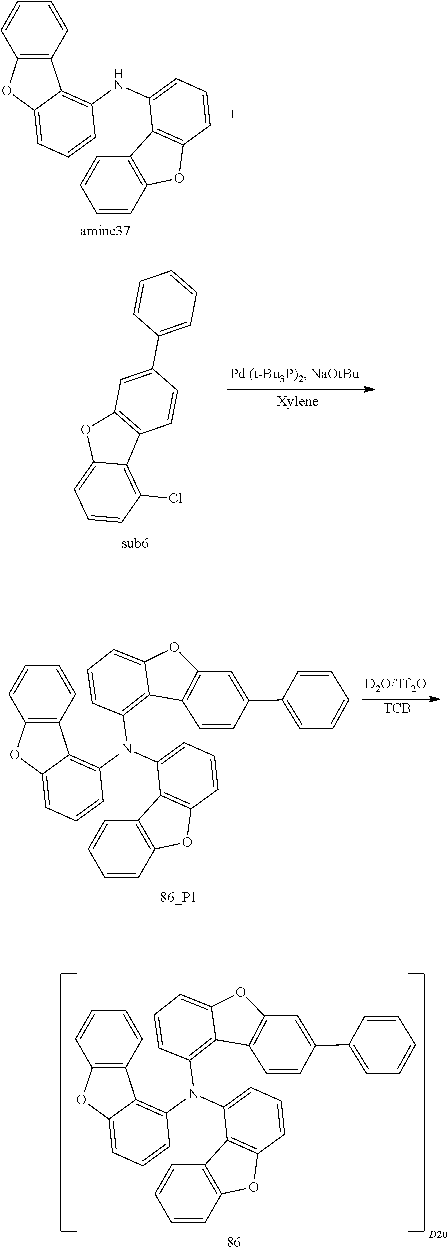

amine37 (15 g, 42.9 mmol), sub6 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 17.5 g of Compound 86_P1. (Yield: 69%, MS: [M+H]+=592)

Compound 86_P1 (10 g, 16.9 mmol) was added to 200 ml of 1,2,4-trichlorobenzene, and stirred at room temperature. In another vessel, deuterium oxide (12.2 g, 609 mmol) was added to trifluoromethanesulfonic anhydride (43 g, 152.2 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.5 g of Compound 86. (Yield: 53%, MS: [M+H]+=612).

### Synthesis Example 87

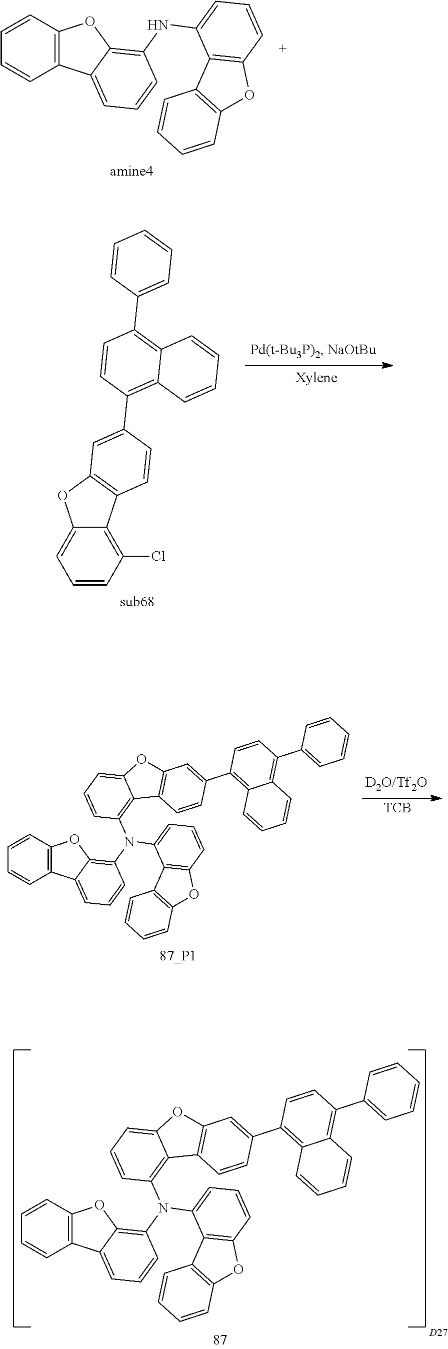

amine4 (15 g, 42.9 mmol), sub68 (18.3 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 16.6 g of Compound 87_P1. (Yield: 54%, MS: [M+H]+=718)

Compound 87_P1 (10 g, 13.9 mmol) was added to 200 ml of 1,2,4-trichlorobenzene, and stirred at room temperature. In another vessel, deuterium oxide (10.1 g, 501.9 mmol) was added to trifluoromethanesulfonic anhydride (35.4 g, 125.5 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.8 g of Compound 87. (Yield: 66%, MS: [M+H]+=745).

### Synthesis Example 88

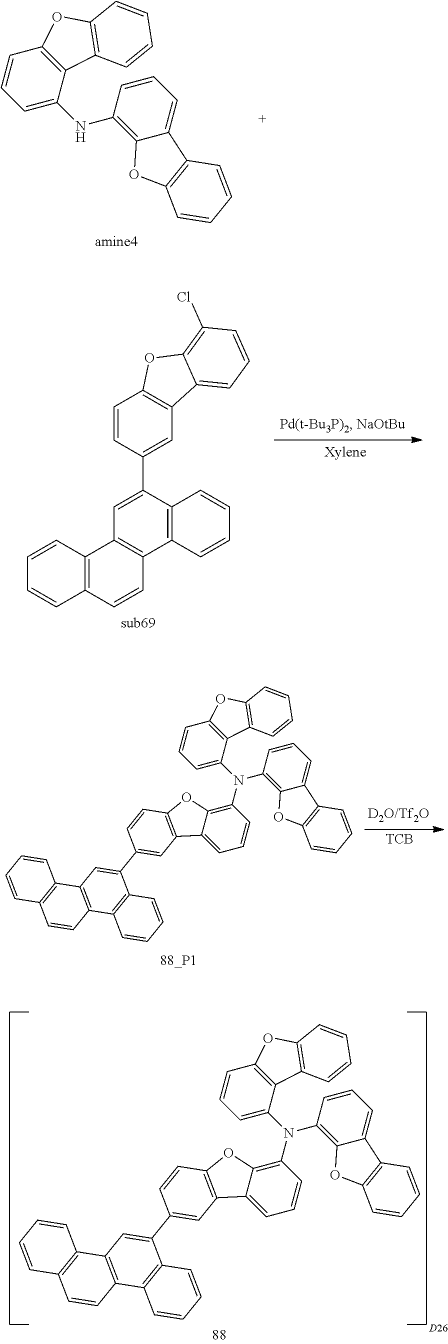

amine4 (15 g, 42.9 mmol), sub69 (19.3 g, 45.1 mmol) and0 sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 22.3 g of Compound 88_P1. (Yield: 70%, MS: [M+H]+=742)

Compound 88_P1 (10 g, 13.5 mmol) was added to 200 ml of 1,2,4-trichlorobenzene, and stirred at room temperature. In another vessel, deuterium oxide (9.7 g, 485.7 mmol) was added to trifluoromethanesulfonic anhydride (34.3 g, 121.4 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 7 g of Compound 88. (Yield: 68%, MS: [M+H]+=768).

### Synthesis Example 89

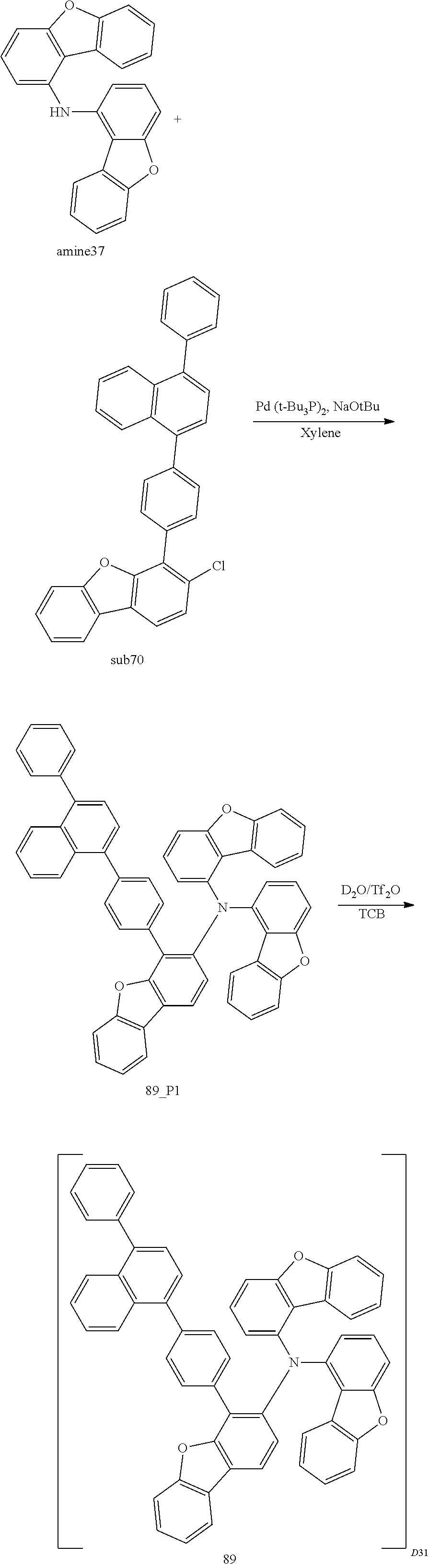

amine37 (15 g, 42.9 mmol), sub70 (21.7 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 21.8 g of Compound 89_P1. (Yield: 64%, MS: [M+H]+=794)

Compound 89_P1 (10 g, 12.6 mmol) was added to 200 ml of 1,2,4-trichlorobenzene, and stirred at room temperature. In another vessel, deuterium oxide (9.1 g, 453.8 mmol) was added to trifluoromethanesulfonic anhydride (32 g, 113.5 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.9 g of Compound 89. (Yield: 57%, MS: [M+H]+=825).

### Synthesis Example 90

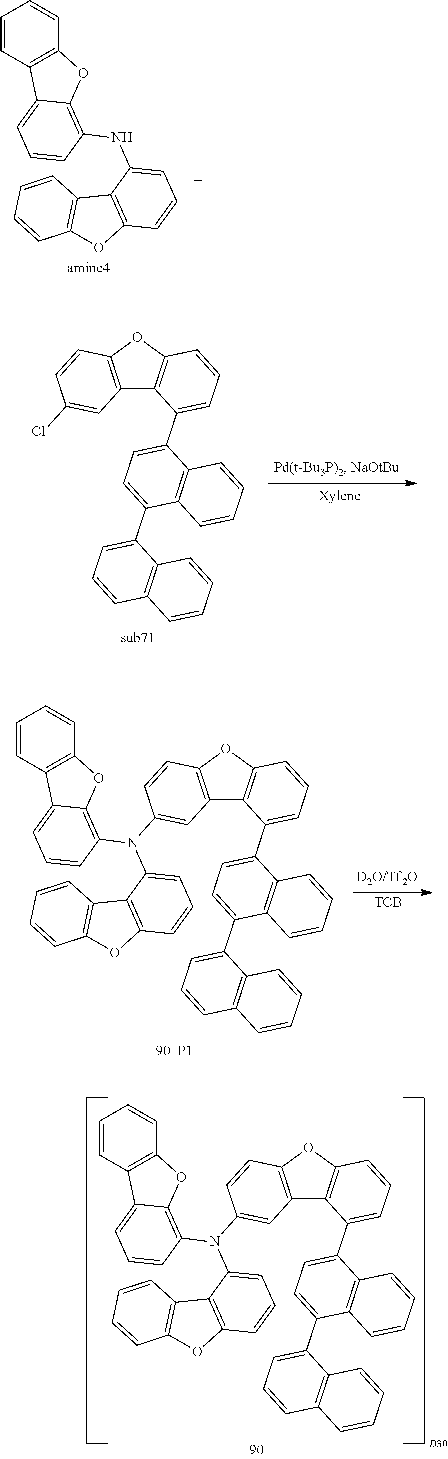

amine4 (15 g, 42.9 mmol), sub71 (20.5 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 19.1 g of Compound 90_P1. (Yield: 58%, MS: [M+H]+=768)

Compound 90_P1 (10 g, 13 mmol) was added to 200 ml of 1,2,4-trichlorobenzene, and stirred at room temperature. In another vessel, deuterium oxide (9.4 g, 469.2 mmol) was added to trifluoromethanesulfonic anhydride (33.1 g, 117.3 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.8 g of Compound 90. (Yield: 56%, MS: [M+H]+=798).

### Synthesis Example 91

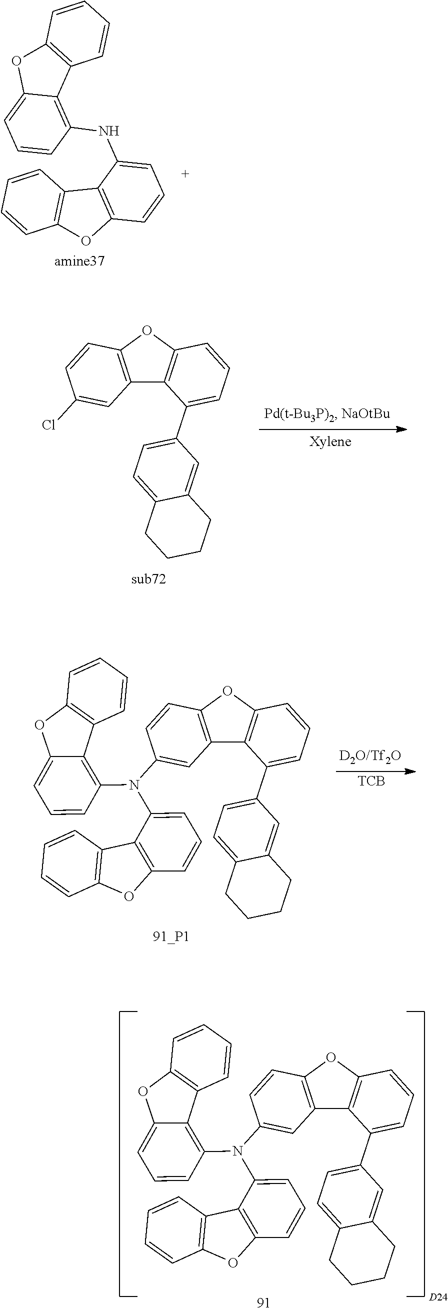

amine37 (15 g, 42.9 mmol), sub72 (15 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 19.4 g of Compound 91_P1. (Yield: 70%, MS: [M+H]+=646)

Compound 91_P1 (10 g, 15.5 mmol) was added to 200 ml of 1,2,4-trichlorobenzene, and stirred at room temperature. In another vessel, deuterium oxide (11.2 g, 557.9 mmol) was added to trifluoromethanesulfonic anhydride (39.4 g, 139.5 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.5 g of Compound 91. (Yield: 53%, MS: [M+H]+=670).

### Synthesis Example 92

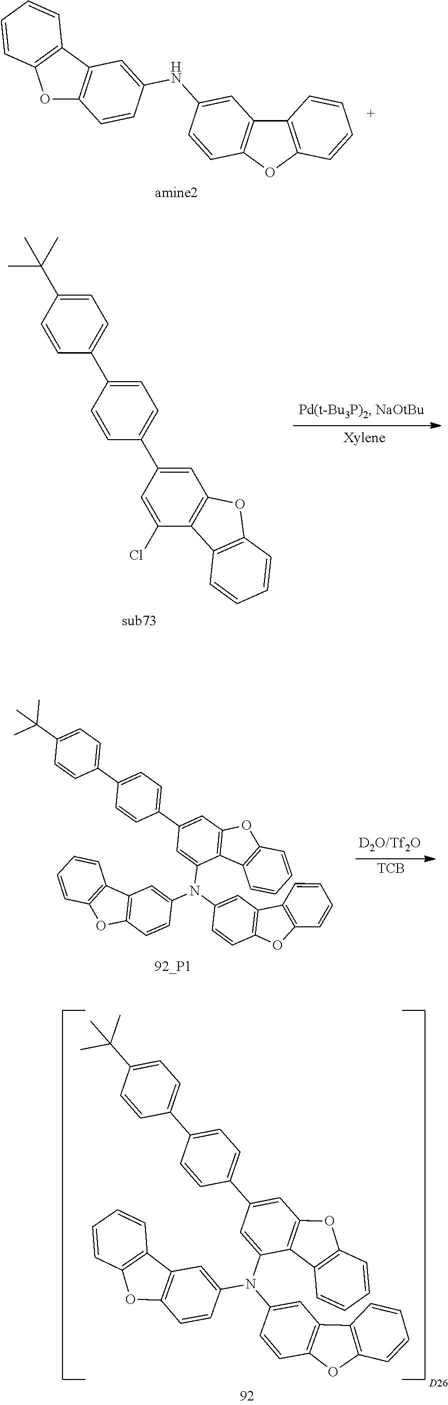

amine2 (15 g, 42.9 mmol), sub73 (18.5 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 18.9 g of Compound 92_P1. (Yield: 61%, MS: [M+H]+=724)

Compound 92_P1 (10 g, 13.8 mmol) was added to 200 ml of 1,2,4-trichlorobenzene, and stirred at room temperature. In another vessel, deuterium oxide (10 g, 497.7 mmol) was added to trifluoromethanesulfonic anhydride (35.1 g, 124.4 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.3 g of Compound 92. (Yield: 51%, MS: [M+H]+=750).

### Synthesis Example 93

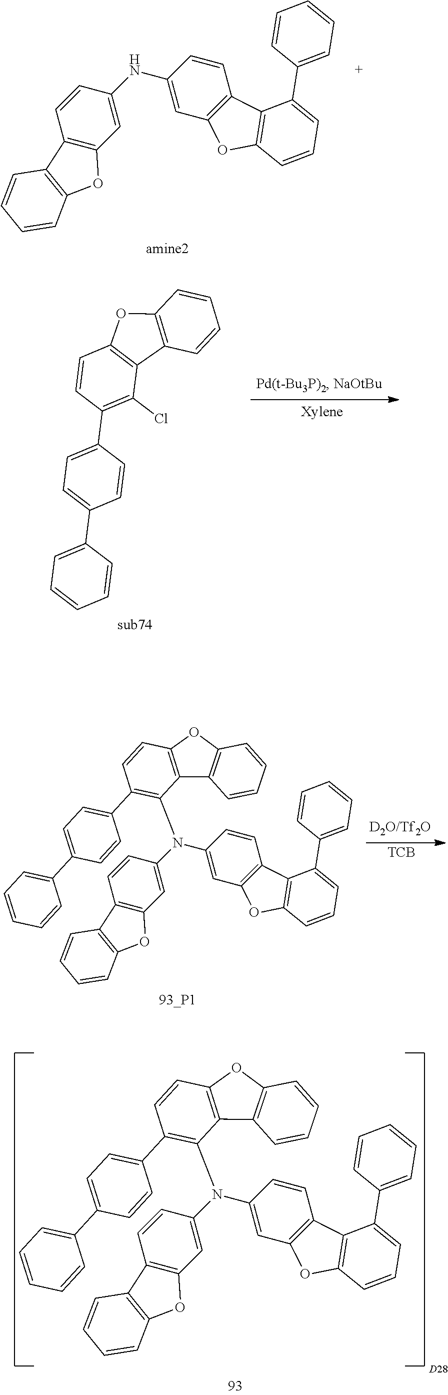

amine2 (15 g, 42.9 mmol), sub74 (16 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 21.1 g of Compound 93_P1. (Yield: 66%, MS: [M+H]+=744)

Compound 93_P1 (10 g, 13.5 mmol) was added to 200 ml of 1,2,4-trichlorobenzene, and stirred at room temperature. In another vessel, deuterium oxide (9.7 g, 484.4 mmol) was added to trifluoromethanesulfonic anhydride (34.2 g, 121.1 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.5 g of Compound 93. (Yield: 63%, MS: [M+H]+=772).

### Synthesis Example 94

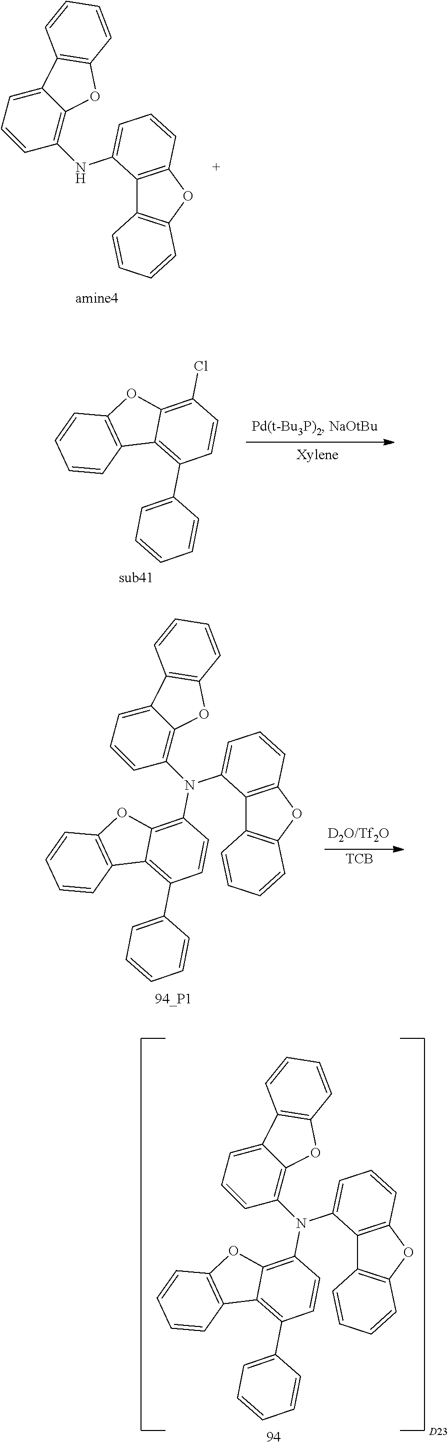

amine4 (15 g, 42.9 mmol), sub41 (12.6 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 13.7 g of Compound 94_P1. (Yield: 54%, MS: [M+H]+=592)

Compound 94_P1 (10 g, 16.9 mmol) was added to 200 ml of 1,2,4-trichlorobenzene, and stirred at room temperature. In another vessel, deuterium oxide (12.2 g, 609 mmol) was added to trifluoromethanesulfonic anhydride (43 g, 152.2 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 7 g of Compound 94. (Yield: 67%, MS: [M+H]+=615).

### Synthesis Example 95

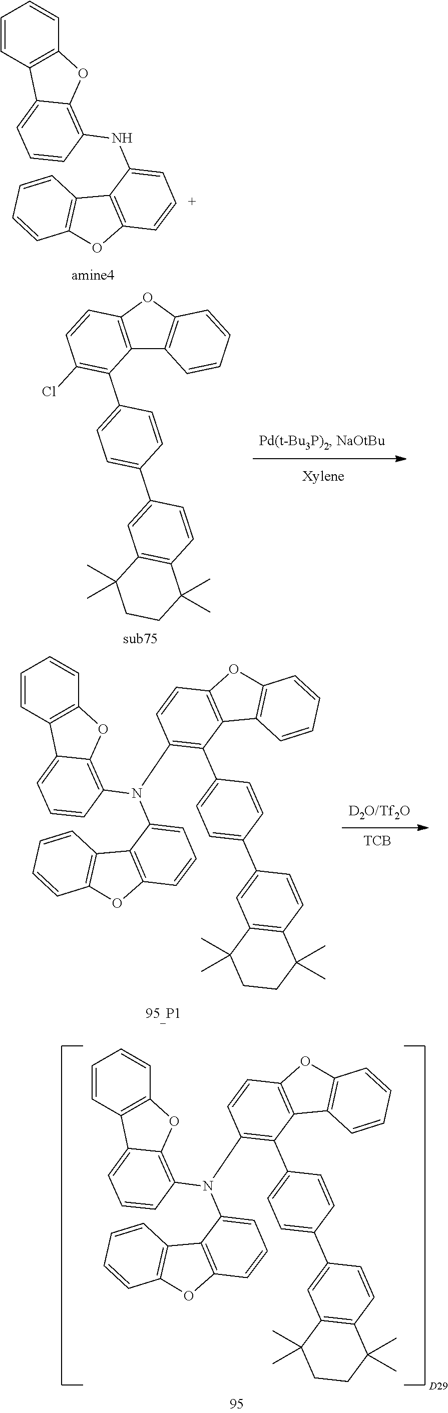

amine4 (15 g, 42.9 mmol), sub75 (21 g, 45.1 mmol) and sodium tert-butoxide (6.2 g, 64.4 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 21.4 g of Compound 95_P1. (Yield: 64%, MS: [M+H]+=778)

Compound 95_P1 (10 g, 12.9 mmol) was added to 200 ml of 1,2,4-trichlorobenzene, and stirred at room temperature. In another vessel, deuterium oxide (9.3 g, 463.1 mmol) was added to trifluoromethanesulfonic anhydride (32.7 g, 115.8 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.3 g of Compound 95. (Yield: 51%, MS: [M+H]+=807).

### Synthesis Example 96

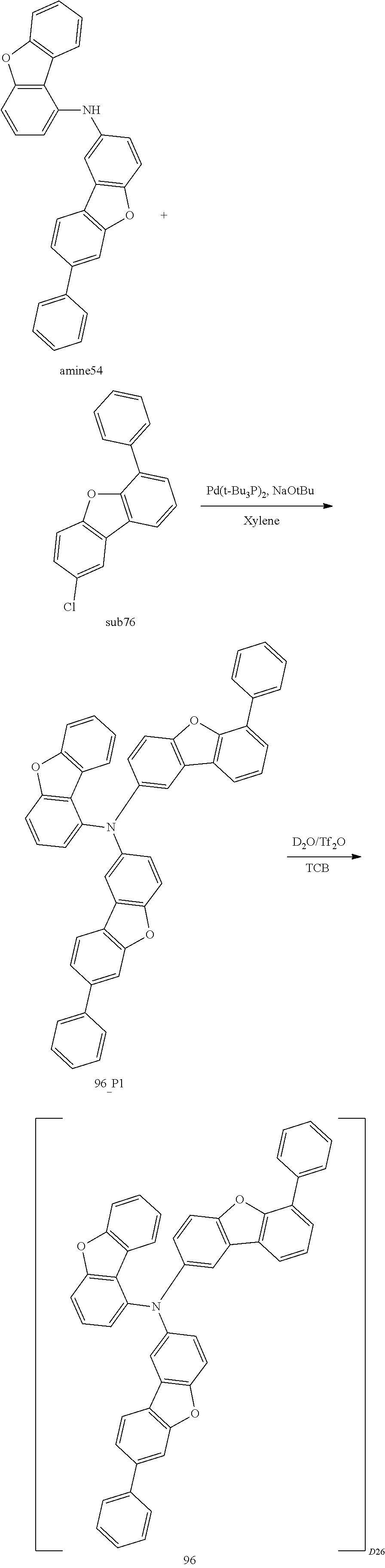

amine54 (15 g, 35.3 mmol), sub76 (10.3 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 12.9 g of Compound 96_P1. (Yield: 55%, MS: [M+H]+=668)

Compound 96_P1 (10 g, 15 mmol) was added to 200 ml of 1,2,4-trichlorobenzene, and stirred at room temperature. In another vessel, deuterium oxide (10.8 g, 539.6 mmol) was added to trifluoromethanesulfonic anhydride (38.1 g, 134.9 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.3 g of Compound 96. (Yield: 51%, MS: [M+H]+=694).

### Synthesis Example 97

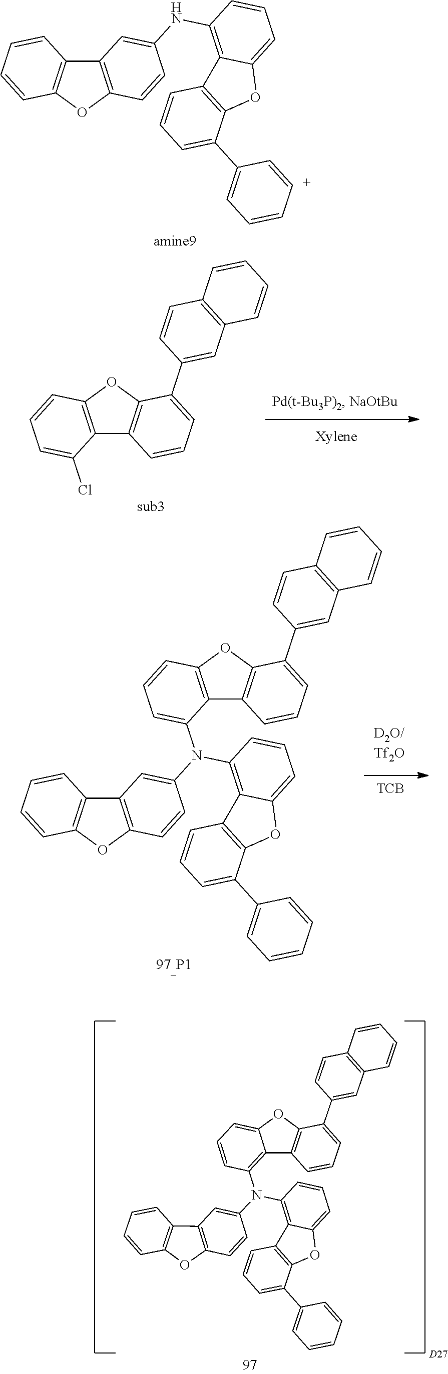

amine9 (15 g, 35.3 mmol), sub3 (12.2 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 17.7 g of Compound 97_P1. (Yield: 70%, MS: [M+H]+=718)

Compound 97_P1 (10 g, 13.9 mmol) was added to 200 ml of 1,2,4-trichlorobenzene, and stirred at room temperature. In another vessel, deuterium oxide (10.1 g, 501.9 mmol) was added to trifluoromethanesulfonic anhydride (35.4 g, 125.5 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 7 g of Compound 97. (Yield: 67%, MS: [M+H]+=745).

### Synthesis Example 98

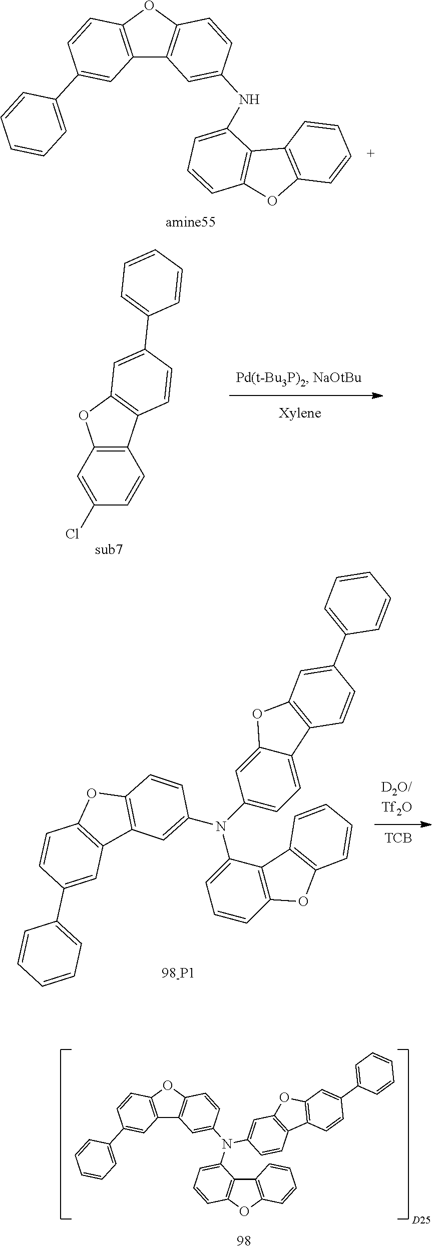

amine55 (15 g, 35.3 mmol), sub7 (10.3 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 13.9 g of Compound 98_P1. (Yield: 59%, MS: [M+H]+=668)

Compound 98_P1 (10 g, 15 mmol) was added to 200 ml of 1,2,4-trichlorobenzene, and stirred at room temperature. In another vessel, deuterium oxide (10.8 g, 539.6 mmol) was added to trifluoromethanesulfonic anhydride (38.1 g, 134.9 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.7 g of Compound 98. (Yield: 65%, MS: [M+H]+=693).

### Synthesis Example 99

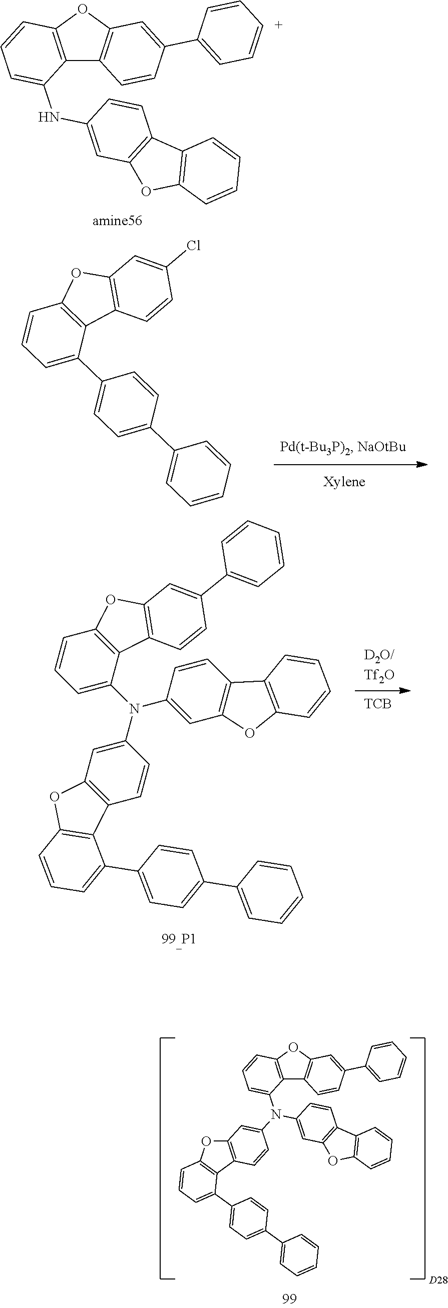

amine56 (15 g, 35.3 mmol), sub77 (13.1 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.1 g of Compound 99_P1. (Yield: 54%, MS: [M+H]+=744)

Compound 99_P1 (10 g, 13.5 mmol) was added to 200 ml of 1,2,4-trichlorobenzene, and stirred at room temperature. In another vessel, deuterium oxide (9.7 g, 484.4 mmol) was added to trifluoromethanesulfonic anhydride (34.2 g, 121.1 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.7 g of Compound 99. (Yield: 55%, MS: [M+H]+=772).

### Synthesis Example 100

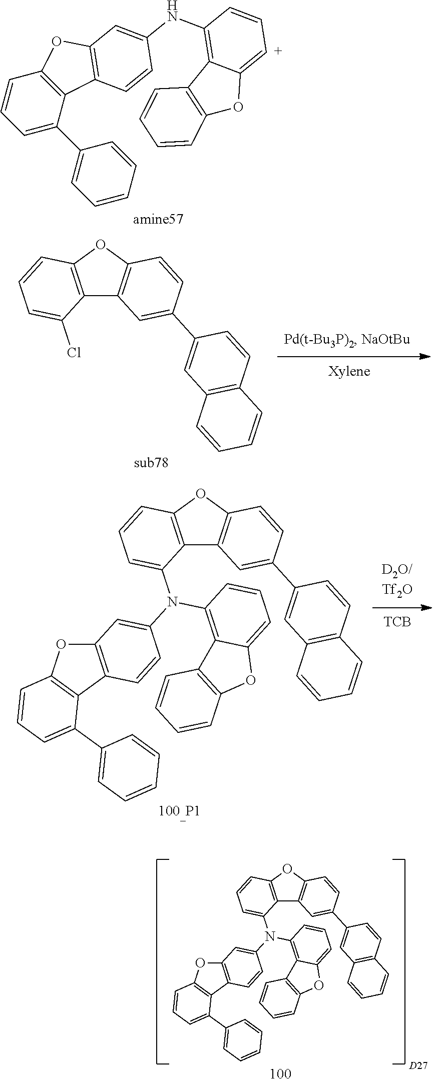

amine57 (15 g, 35.3 mmol), sub78 (12.2 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 13.7 g of Compound 100_P1. (Yield: 54%, MS: [M+H]+=718)

Compound 100_P1 (10 g, 13.9 mmol) was added to 200 ml of 1,2,4-trichlorobenzene, and stirred at room temperature. In another vessel, deuterium oxide (10.1 g, 501.9 mmol) was added to trifluoromethanesulfonic anhydride (35.4 g, 125.5 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.7 g of Compound 100. (Yield: 65%, MS: [M+H]+=745).

### Synthesis Example 101

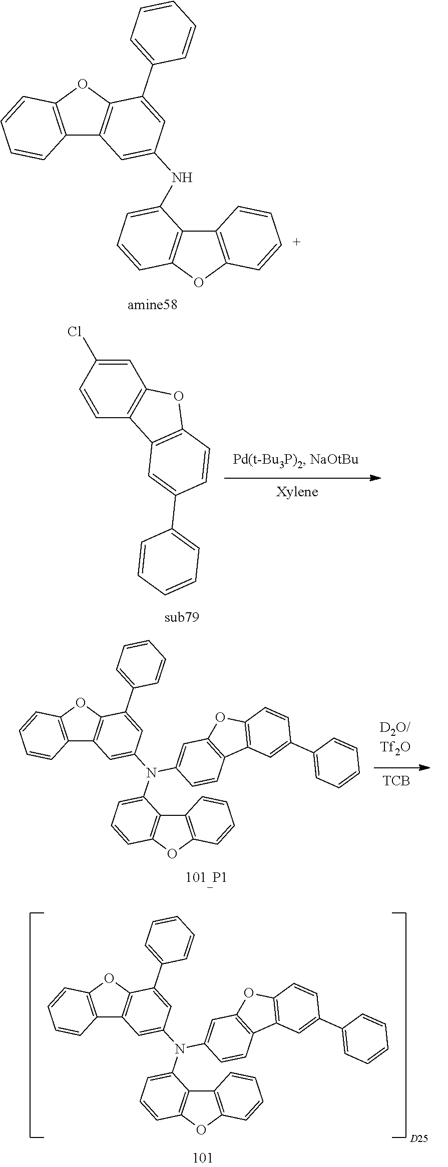

amine58 (15 g, 35.3 mmol), sub79 (10.3 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 12.5 g of Compound 101_P1. (Yield: 53%, MS: [M+H]+=668)

Compound 101_P1 (10 g, 15 mmol) was added to 200 ml of 1,2,4-trichlorobenzene, and stirred at room temperature. In another vessel, deuterium oxide (10.8 g, 539.6 mmol) was added to trifluoromethanesulfonic anhydride (38.1 g, 134.9 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.1 g of Compound 101. (Yield: 59%, MS: [M+H]+=693).

### Synthesis Example 102

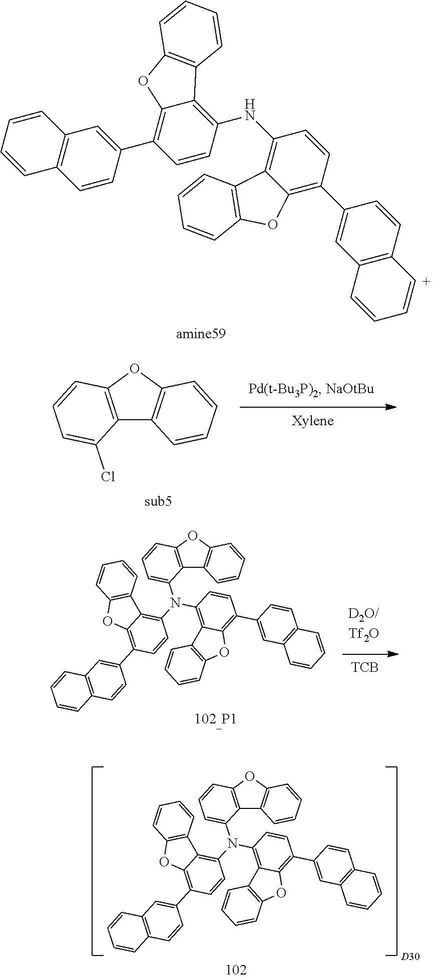

amine59 (15 g, 24.9 mmol), sub5 (5.3 g, 26.2 mmol) and sodium tert-butoxide (3.6 g, 37.4 mmol) were added to 300 ml of xylene under a nitrogen atmosphere, and mixture was stirred and refluxed. Then, bis(tri-tert-butylphosphine)palladium(0) (0.1 g, 0.2 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 11.3 g of Compound 102_P1. (Yield: 59%, MS: [M+H]+=768)

Compound 102_P1 (10 g, 13 mmol) was added to 200 ml of 1,2,4-trichlorobenzene, and stirred at room temperature. In another vessel, deuterium oxide (9.4 g, 469.2 mmol) was added to trifluoromethanesulfonic anhydride (33.1 g, 117.3 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.7 g of Compound 102. (Yield: 55%, MS: [M+H]+=798).

### Synthesis Example 103

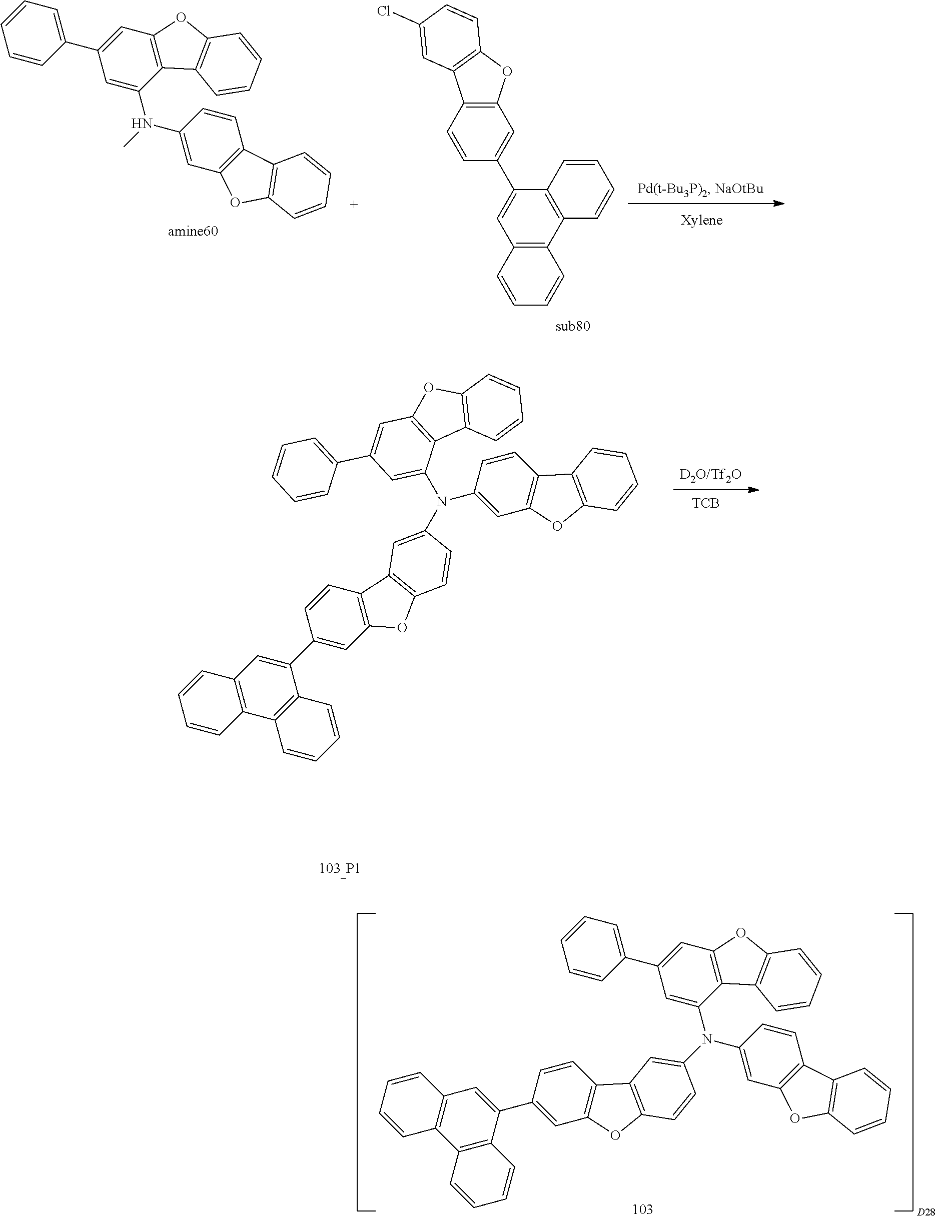

amine60 (15 g, 35.3 mmol), sub80 (14 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.6 g of Compound 103_P1. (Yield: 54%, MS: [M+H]+=768)

Compound 103_P1 (10 g, 13 mmol) was added to 200 ml of 1,2,4-trichlorobenzene, and stirred at room temperature. In another vessel, deuterium oxide (9.4 g, 469.2 mmol) was added to trifluoromethanesulfonic anhydride (33.1 g, 117.3 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.4 g of Compound 103. (Yield: 52%, MS: [M+H]+=796).

### Synthesis Example 104

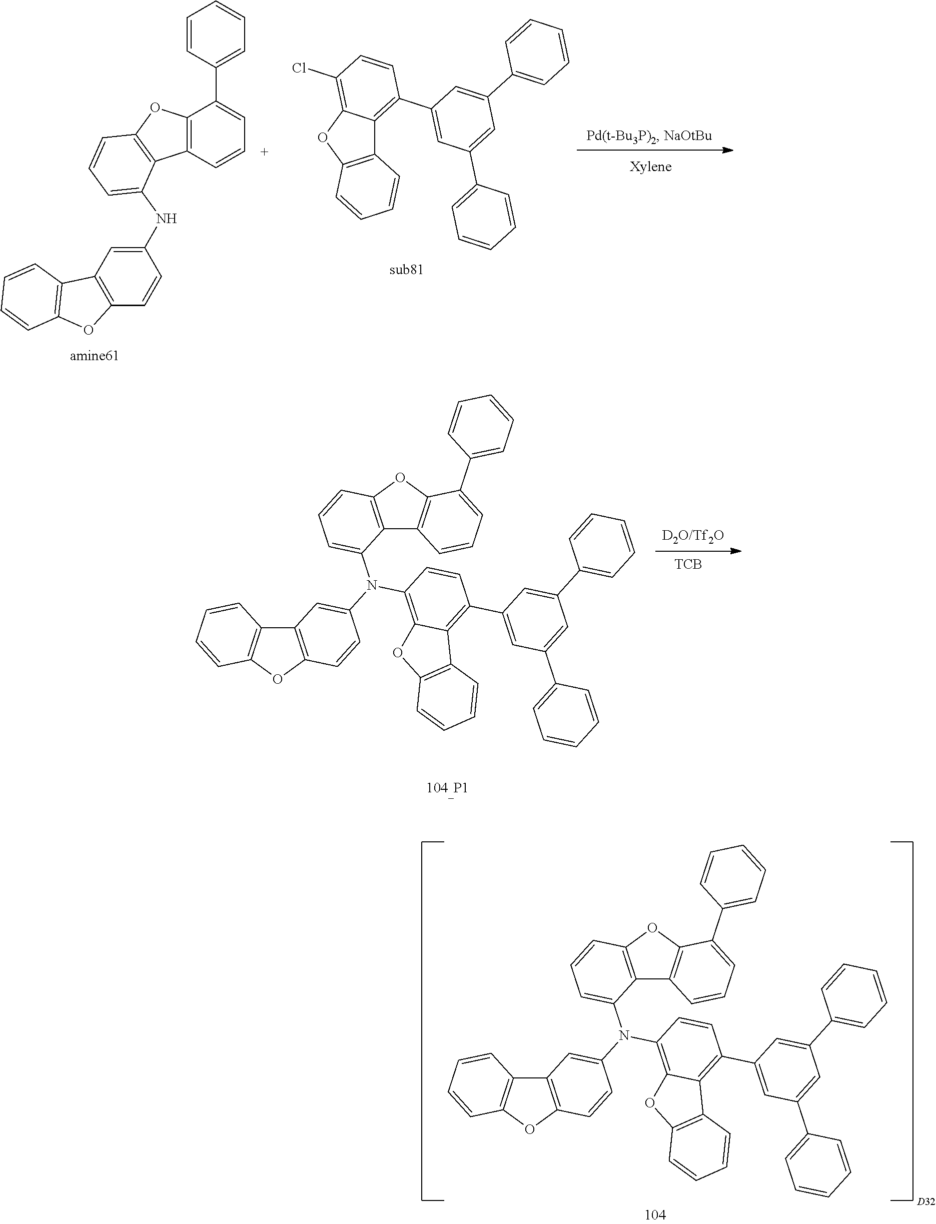

amine61 (15 g, 35.3 mmol), sub81 (16 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 19.1 g of Compound 104_P1. (Yield: 66%, MS: [M+H]+=820)

Compound 104_P1 (10 g, 12.2 mmol) was added to 200 ml of 1,2,4-trichlorobenzene, and stirred at room temperature. In another vessel, deuterium oxide (8.8 g, 439.4 mmol) was added to trifluoromethanesulfonic anhydride (31 g, 109.9 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 7.3 g of Compound 104. (Yield: 70%, MS: [M+H]+=852).

### Synthesis Example 105

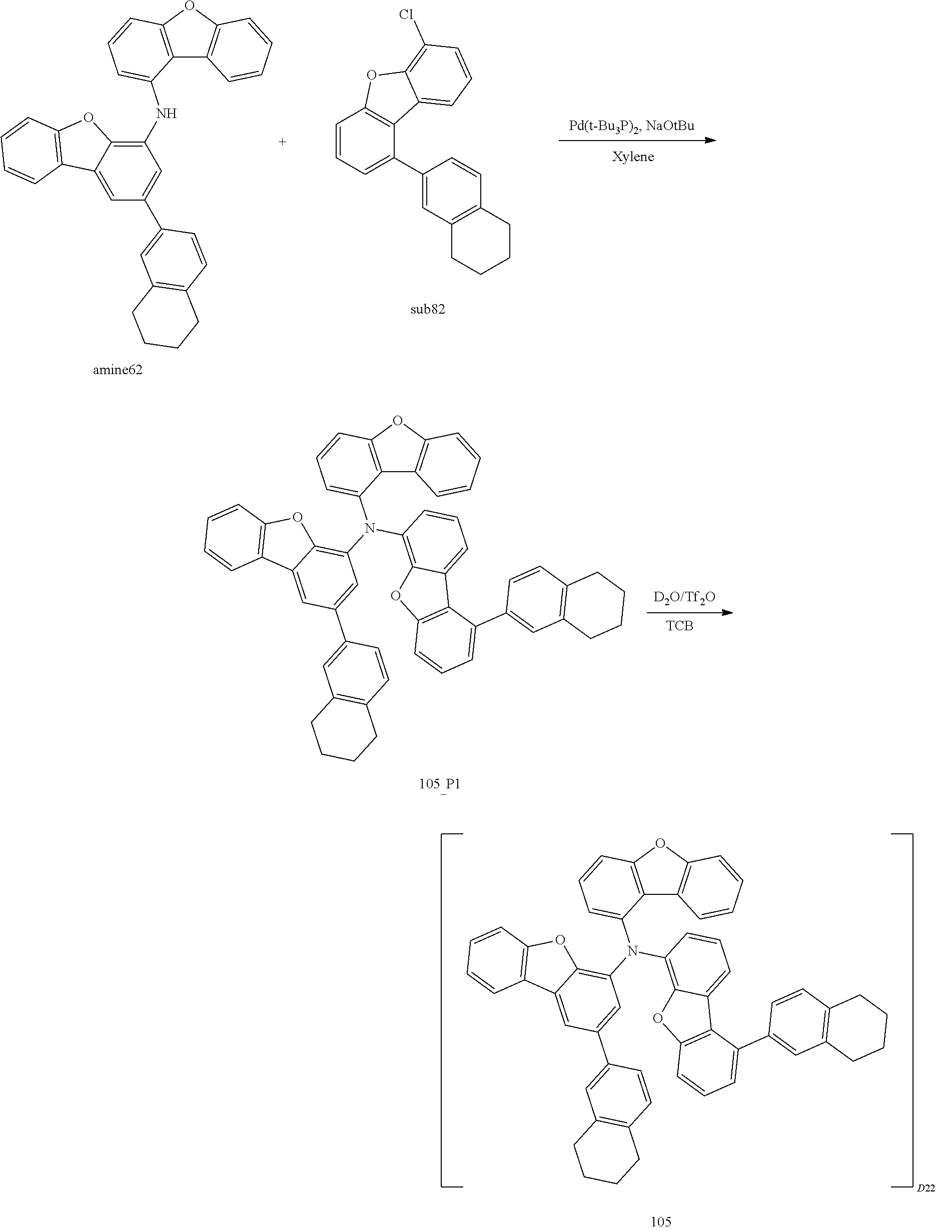

amine62 (15 g, 31.3 mmol), sub82 (10.9 g, 32.8 mmol) and sodium tert-butoxide (4.5 g, 46.9 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 14.3 g of Compound 105_P1. (Yield: 59%, MS: [M+H]+=776)

Compound 105_P1 (10 g, 12.9 mmol) was added to 200 ml of 1,2,4-trichlorobenzene, and stirred at room temperature. In another vessel, deuterium oxide (9.3 g, 464.3 mmol) was added to trifluoromethanesulfonic anhydride (32.8 g, 116.1 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.4 g of Compound 105. (Yield: 62%, MS: [M+H]+=798).

### Synthesis Example 106

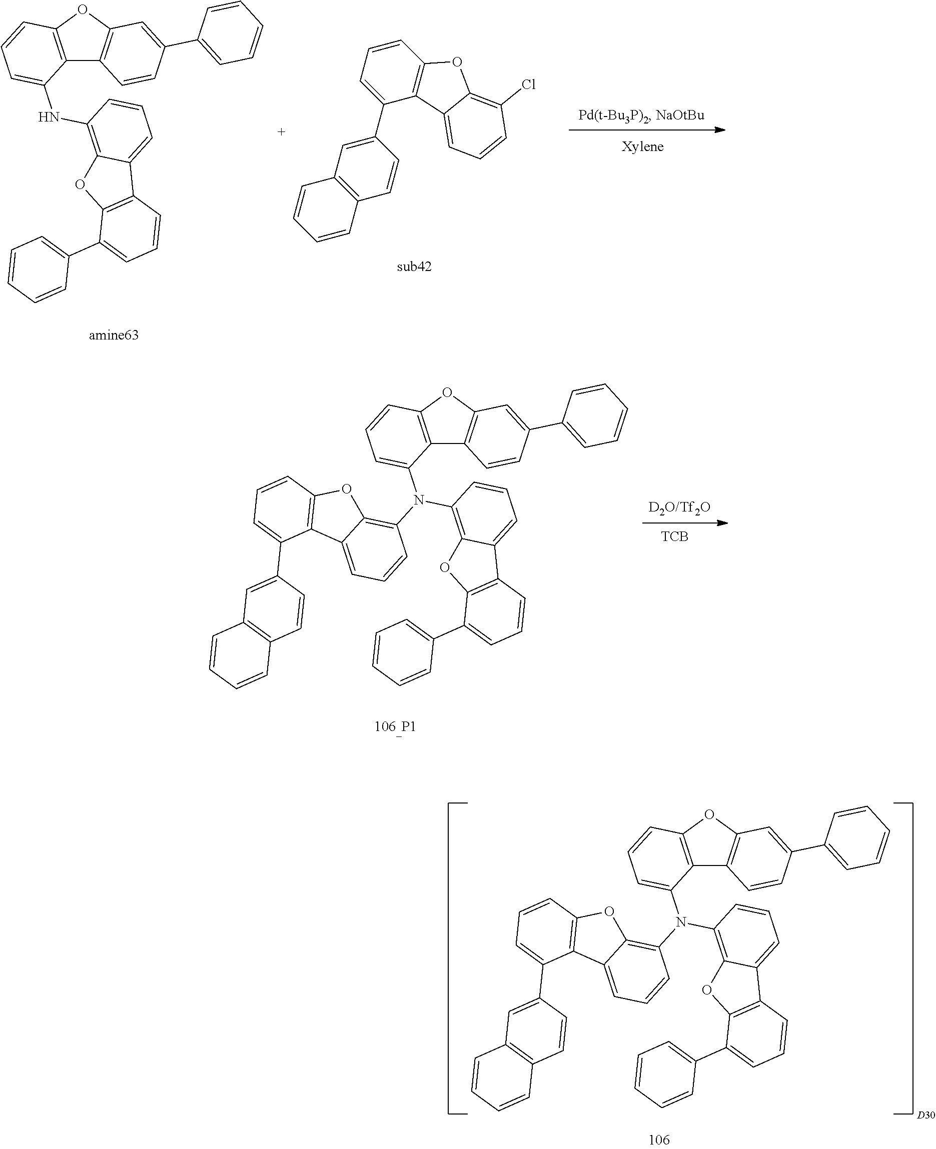

amine63 (15 g, 29.9 mmol), sub42 (10.3 g, 31.4 mmol) and sodium tert-butoxide (4.3 g, 44.9 mmol) were added to 300 ml of xylene under a nitrogen atmosphere, and mixture was stirred and refluxed. Then, bis(tri-tert-butylphosphine)palladium(0) (0.2 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15.4 g of Compound 106_P1. (Yield: 65%, MS: [M+H]+=794)

Compound 106_P1 (10 g, 12.6 mmol) was added to 200 ml of 1,2,4-trichlorobenzene, and stirred at room temperature. In another vessel, deuterium oxide (9.1 g, 453.8 mmol) was added to trifluoromethanesulfonic anhydride (32 g, 113.5 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 5.6 g of Compound 106. (Yield: 54%, MS: [M+H]+=824).

### Synthesis Example 107

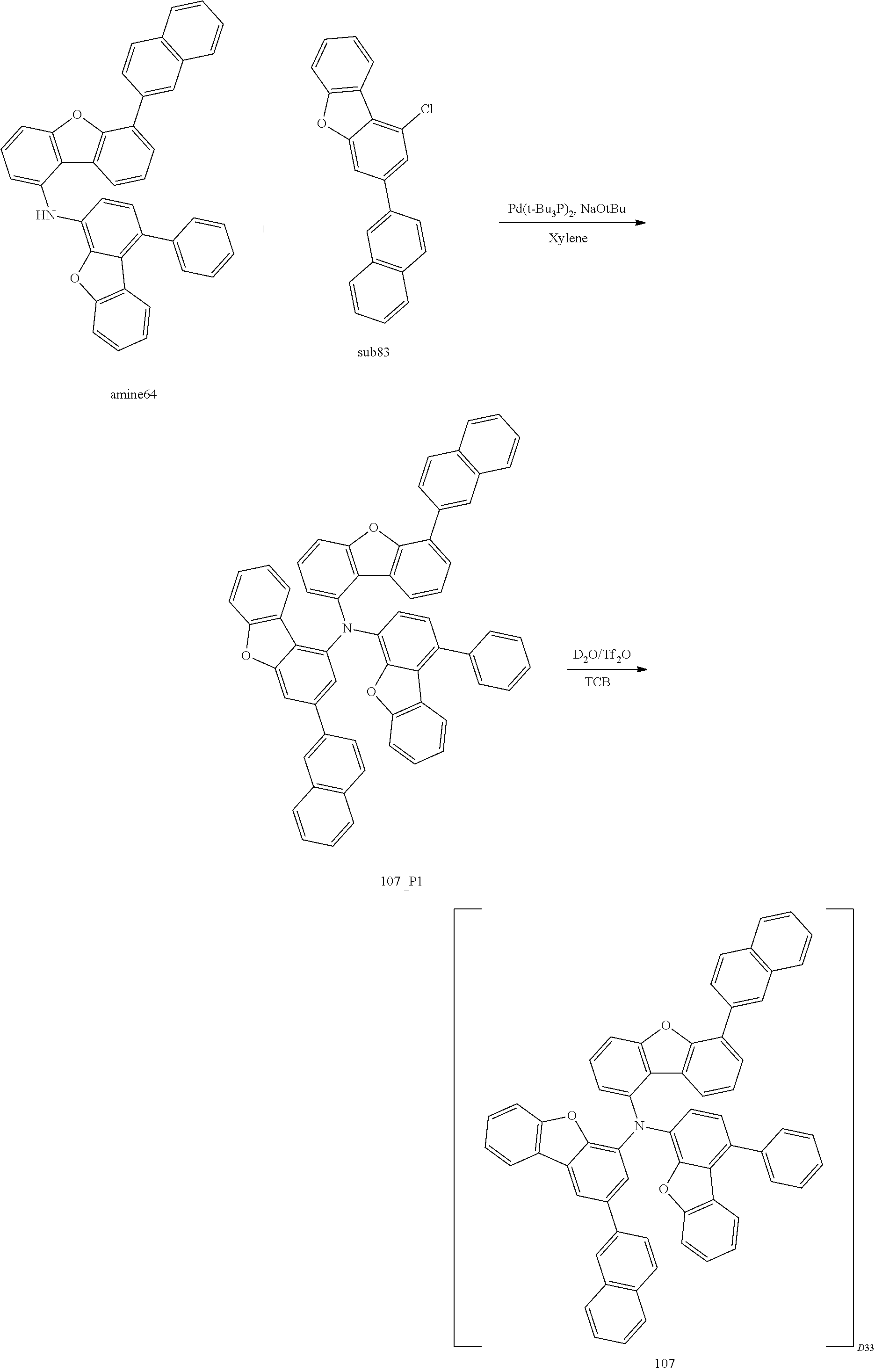

amine64 (15 g, 27.2 mmol), sub83 (9.4 g, 28.6 mmol) and sodium tert-butoxide (3.9 g, 40.8 mmol) were added to 300 ml of xylene under a nitrogen atmosphere, and mixture was stirred and refluxed. Then, bis(tri-tert-butylphosphine)palladium(0) (0.1 g, 0.3 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 15.4 g of Compound 107_P1. (Yield: 67%, MS: [M+H]+=844)

Compound 107_P1 (10 g, 11.9 mmol) was added to 200 ml of 1,2,4-trichlorobenzene, and stirred at room temperature. In another vessel, deuterium oxide (8.6 g, 426.9 mmol) was added to trifluoromethanesulfonic anhydride (30.1 g, 106.7 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.3 g of Compound 107. (Yield: 61%, MS: [M+H]+=877).

### Synthesis Example 108

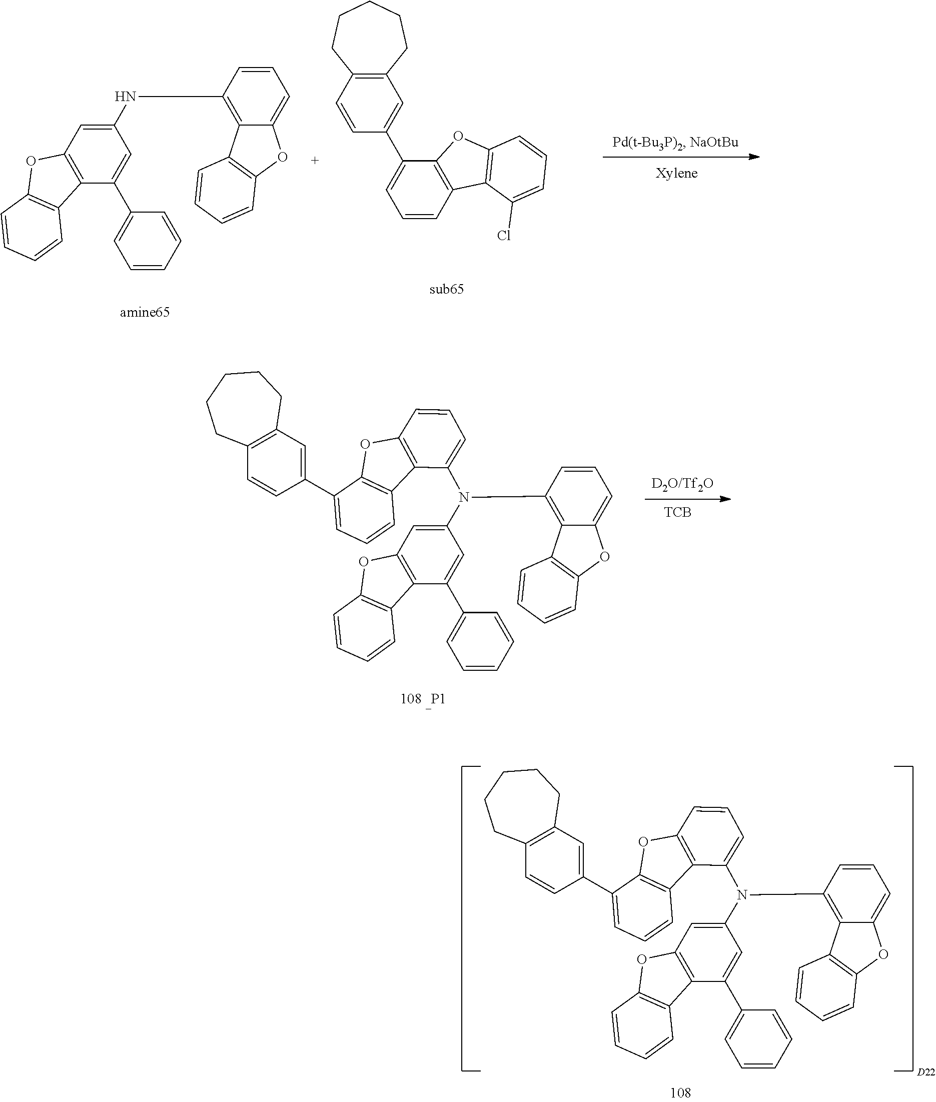

amine65 (15 g, 35.3 mmol), sub65 (12.8 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 17.6 g of Compound 108_P1. (Yield: 68%, MS: [M+H]+=736)

Compound 108_P1 (10 g, 13.6 mmol) was added to 200 ml of 1,2,4-trichlorobenzene, and stirred at room temperature. In another vessel, deuterium oxide (9.8 g, 489.6 mmol) was added to trifluoromethanesulfonic anhydride (34.5 g, 122.4 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 7 g of Compound 108. (Yield: 68%, MS: [M+H]+=758).

### Synthesis Example 109

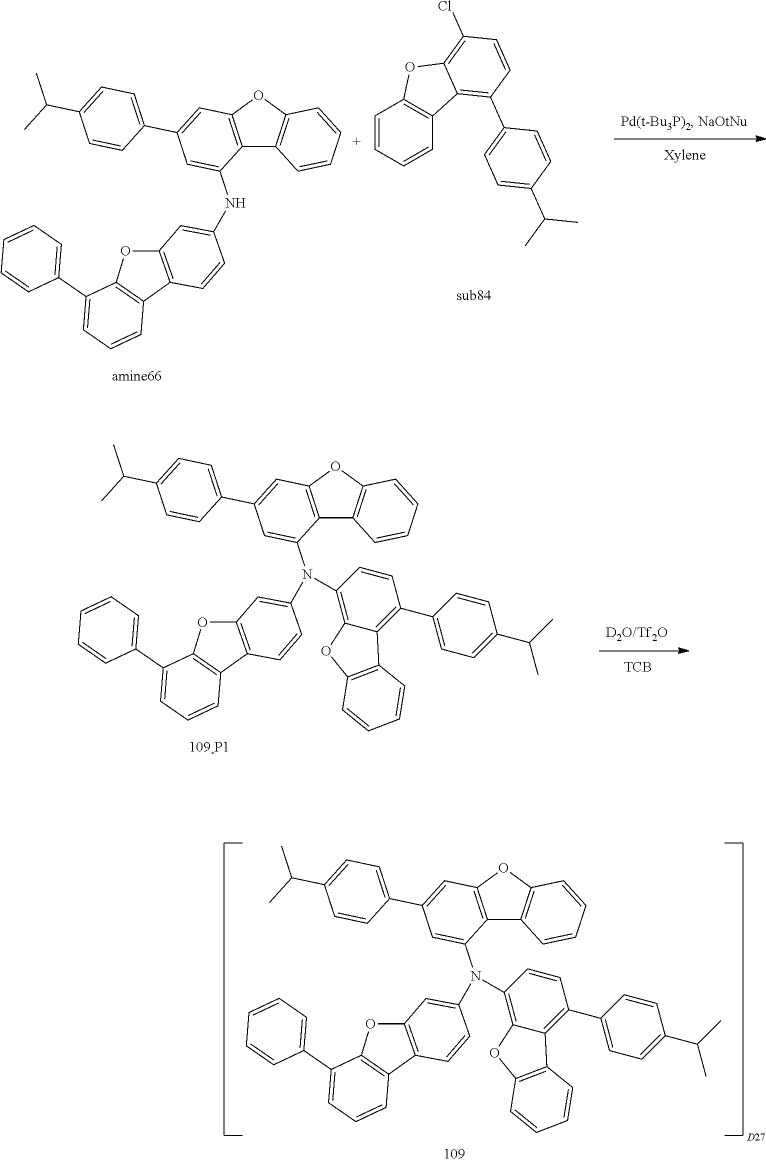

amine66 (15 g, 35.3 mmol), sub84 (11.9 g, 37 mmol) and sodium tert-butoxide (5.1 g, 52.9 mmol) were added to 300 ml of xylene under a nitrogen atmosphere, and mixture was stirred and refluxed. Then, bis(tri-tert-butylphosphine)palladium(0) (0.2 g, 0.4 mmol) was added thereto. The reaction was completed after 5 hours, the reaction mixture was cooled to room temperature, and the solvent was removed under reduced pressure. Then, the compound was again completely dissolved in chloroform, washed twice with water, and then the organic layer was separated, treated with anhydrous magnesium sulfate, then filtered, and the filtrate was distilled under reduced pressure. The concentrated compound was purified by a silica gel column chromatography to prepare 16.6 g of Compound 109_P1. (Yield: 57%, MS: [M+H]+=828)

Compound 109_P1 (10 g, 12.1 mmol) was added to 200 ml of 1,2,4-trichlorobenzene, and stirred at room temperature. In another vessel, deuterium oxide (8.7 g, 435.1 mmol) was added to trifluoromethanesulfonic anhydride (30.7 g, 108.8 mmol) at 0° C., and the mixture was stirred for 5 hours to prepare a solution. Then, a mixed solution of trifluoromethanesulfonic anhydride and deuterium oxide was added dropwise to the prepared mixed solution of 1,2,4-trichlorobenzene, and the mixture was stirred while heating to 140° C. and then keeping that temperature. After the reaction for 10 hours, the reaction mixture was cooled to room temperature, and the organic layer and the aqueous layer were separated. Then, the organic layer was neutralized with an aqueous potassium carbonate solution. After washing twice with water, the organic layer was separated, anhydrous magnesium sulfate was added thereto, stirred, then filtered, and the filtrate was distilled under reduced pressure. The concentrated compound was purified by a silica gel column chromatography to prepare 6.1 g of Compound 109. (Yield: 59%, MS: [M+H]+=855).

### EXAMPLES

### Example 1-1

A glass substrate on which ITO (indium tin oxide) was coated as a thin film to a thickness of 1,000 Å was put into distilled water in which a detergent was dissolved, and ultrasonically cleaned. A product manufactured by Fischer Co. was used as the detergent, and as the distilled water, distilled water filtered twice using a filter manufactured by Millipore Co. was used. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. In addition, the substrate was cleaned for 5 minutes using oxygen plasma and then transferred to a vacuum depositor.

On the ITO transparent electrode thus prepared, the following compound HI-1 was formed in a thickness of 1100 Å as a hole injection layer, but the following compound A-1 was p-doped at a concentration of 1.5 wt. %. The following compound HT-1 was vacuum deposited on the hole injection layer to form a hole transport layer with a layer thickness of 800 Å. Then, the compound 1 prepared in Synthesis Example 1 was thermally vacuum deposited as the hole transport auxiliary layer to a thickness of 100 Å. Then, the compounds represented by the following Chemical Formulas BH-1 and BD-1 were vacuum deposited in a weight ratio of 25:1 as a light emitting layer to a thickness of 250 Å. Next, Compound represented by the following Chemical Formula HB-1 was vacuum deposited to a thickness of 50 Å as a hole blocking layer. Next, the compound represented by the following Chemical Formula ET-1 and the compound represented by LiQ were thermally vacuum deposited at a weight ratio of 1:1 to a thickness of 310 Å as an electron injection and transport layer. Lithium fluoride (LiF) and aluminum were sequentially deposited to have a thickness of 12 Å and 1,000 Å, respectively, on the electron injection and transport layer to form a cathode, thereby completing the manufacture of an organic light emitting device.

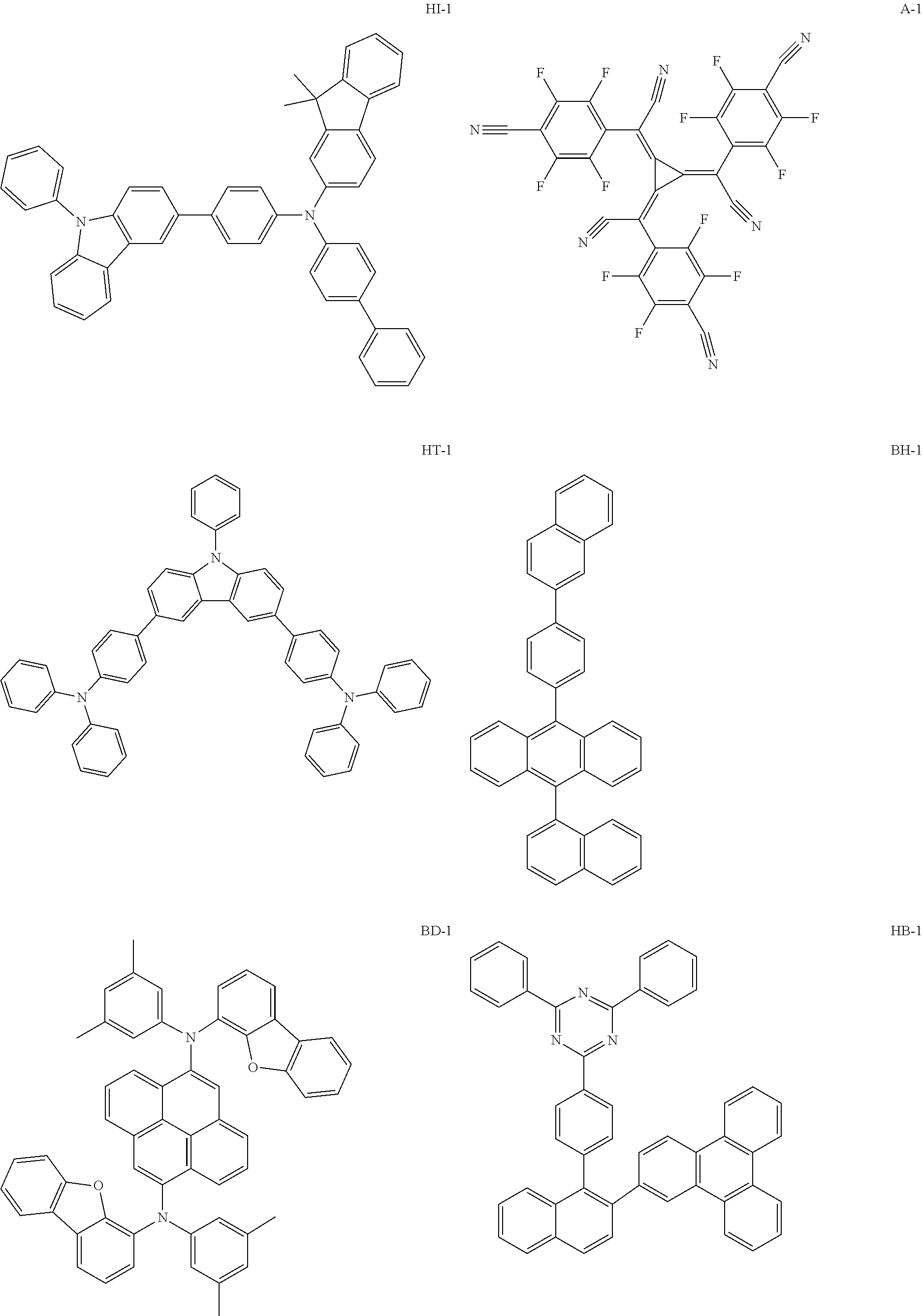

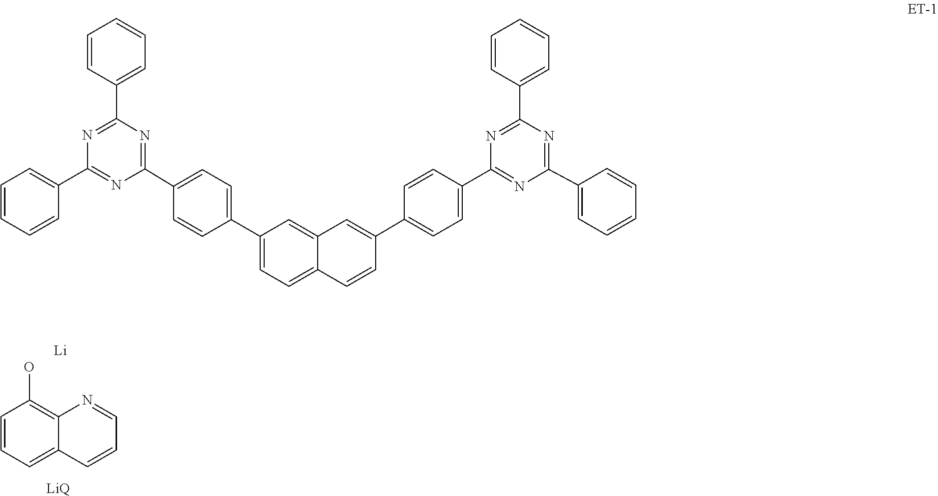

In the above-mentioned processes, the deposition rates of the organic materials were maintained at 0.4˜0.7 Å/sec, the deposition rates of magnesium and silver were maintained at 0.3 Å/sec and 2 Å/sec, respectively, and the degree of vacuum during the deposition was maintained at 2×10⁻⁷˜5×10⁻⁶ torr, thereby manufacturing an organic light emitting device.

### Examples 1-2 to 1-109

The organic light emitting devices of Examples 1-2 to 1-109 were manufactured in the same manner as in Example 1-1, except that the compounds listed in Table 1 below were used instead of Compound 1.

### Comparative Examples 1-1 to 1-8

The organic light emitting devices of Comparative Examples 1-1 to 1-8 were manufactured in the same manner as in Example 1-1, except that the compounds listed in Table 1 below were used instead of Compound 1.

The compounds C-1 to C-8 used in Comparative Examples 1-1 to 1-8 are as follows.

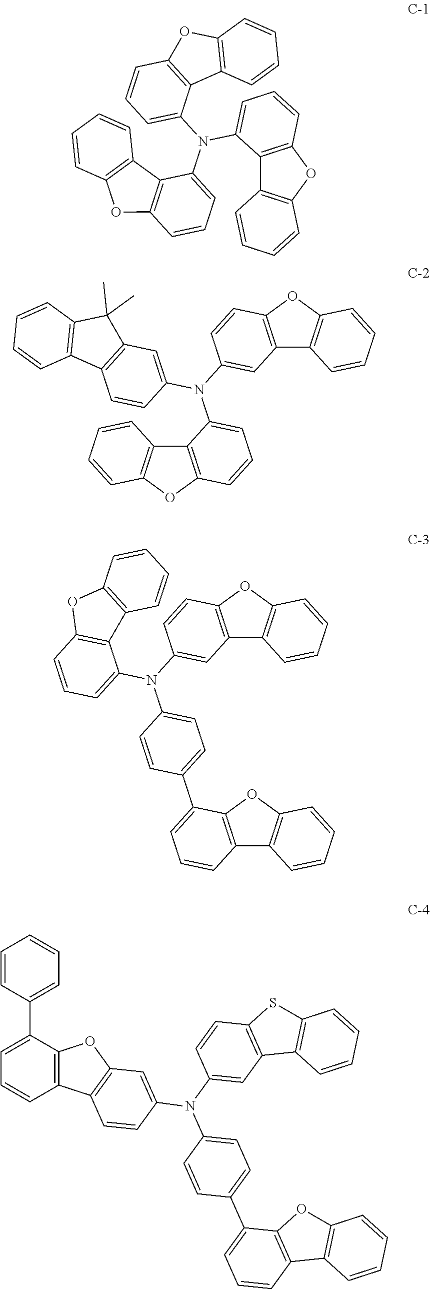

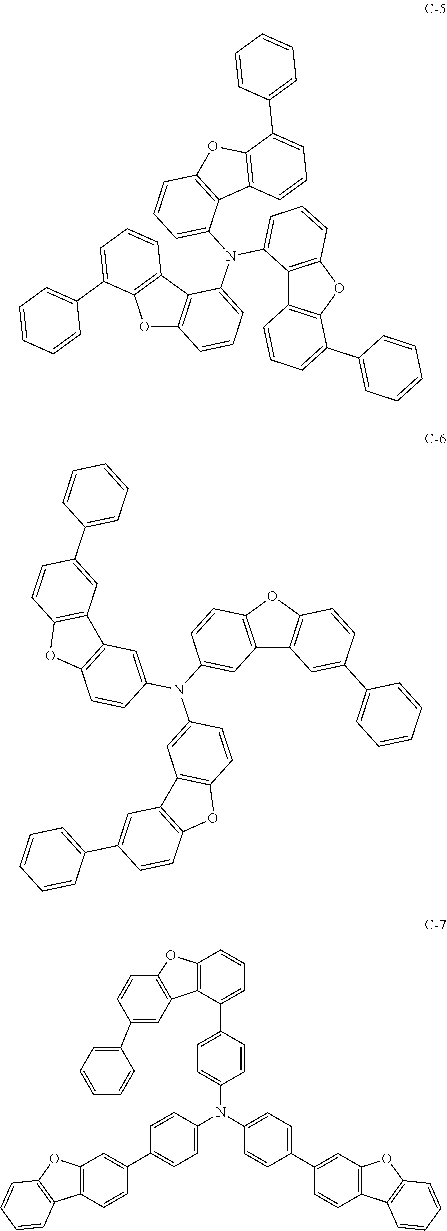 [SMILES: c1ccc(-c2cccc3c2oc2cccc(N(c4cccc5oc6c(-c7ccccc7)cccc6c45)c4cccc5oc6c(-c7ccccc7)cccc6c45)c23)cc1 ; c1ccc(-c2ccc3oc4ccc(N(c5ccc6oc7ccc(-c8ccccc8)cc7c6c5)c5ccc6oc7ccc(-c8ccccc8)cc7c6c5)cc4c3c2)cc1 ; c1ccc(-c2ccc3oc4cccc(-c5ccc(N(c6ccc(-c7ccc8c(c7)oc7ccccc78)cc6)c6ccc(-c7ccc8c(c7)oc7ccccc78)cc6)cc5)c4c3c2)cc1]

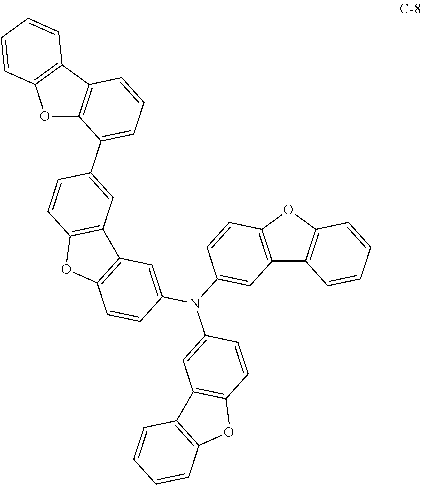 [SMILES: c1ccc2c(c1)oc1ccc(N(c3ccc4oc5ccccc5c4c3)c3ccc4oc5ccc(-c6cccc7c6oc6ccccc67)cc5c4c3)cc12]

### Experimental Example

The voltage and efficiency were measured by applying a current of 15 mA/cm² to the organic light emitting devices manufactured in Examples 1-1 to 1-109 and Comparative Examples 1-1 to 1-8, and the results are shown in Table 1 below. Lifetime T95 means the time required for the luminance to be reduced to 95% of the initial luminance (1000 nit).

TABLE 1

| | | | Effi- | Lifetime | Lumi- |
|---|---|---|---|---|---|
| | | Voltage | ciency | T95 | nescent |
| | Compound | (V) | (cd/A) | (hr) | color |
| Example 1-1 | Compound 1 | 3.31 | 5.23 | 158 | Blue |
| Example 1-2 | Compound 2 | 3.38 | 5.36 | 172 | Blue |
| Example 1-3 | Compound 3 | 3.25 | 5.12 | 186 | Blue |
| Example 1-4 | Compound 4 | 3.22 | 5.08 | 193 | Blue |
| Example 1-5 | Compound 5 | 3.22 | 5.09 | 182 | Blue |
| Example 1-6 | Compound 6 | 3.22 | 5.17 | 190 | Blue |
| Example 1-7 | Compound 7 | 3.24 | 5.05 | 196 | Blue |
| Example 1-8 | Compound 8 | 3.33 | 5.25 | 157 | Blue |
| Example 1-9 | Compound 9 | 3.36 | 5.22 | 161 | Blue |
| Example 1-10 | Compound 10 | 3.55 | 5.35 | 183 | Blue |
| Example 1-11 | Compound 11 | 3.51 | 5.27 | 192 | Blue |
| Example 1-12 | Compound 12 | 3.51 | 5.37 | 177 | Blue |
| Example 1-13 | Compound 13 | 3.49 | 5.35 | 191 | Blue |
| Example 1-14 | Compound 14 | 3.55 | 5.34 | 180 | Blue |
| Example 1-15 | Compound 15 | 3.37 | 5.34 | 159 | Blue |
| Example 1-16 | Compound 16 | 3.36 | 5.22 | 169 | Blue |
| Example 1-17 | Compound 17 | 3.21 | 5.15 | 192 | Blue |
| Example 1-18 | Compound 18 | 3.29 | 5.06 | 186 | Blue |
| Example 1-19 | Compound 19 | 3.23 | 5.17 | 191 | Blue |
| Example 1-20 | Compound 20 | 3.27 | 5.11 | 197 | Blue |
| Example 1-21 | Compound 21 | 3.23 | 5.05 | 178 | Blue |
| Example 1-22 | Compound 22 | 3.25 | 5.12 | 190 | Blue |
| Example 1-23 | Compound 23 | 3.26 | 5.10 | 182 | Blue |
| Example 1-24 | Compound 24 | 3.53 | 5.37 | 189 | Blue |
| Example 1-25 | Compound 25 | 3.31 | 5.33 | 163 | Blue |
| Example 1-26 | Compound 26 | 3.41 | 5.27 | 158 | Blue |
| Example 1-27 | Compound 27 | 3.55 | 5.35 | 179 | Blue |
| Example 1-28 | Compound 28 | 3.52 | 5.33 | 184 | Blue |
| Example 1-29 | Compound 29 | 3.25 | 5.19 | 195 | Blue |
| Example 1-30 | Compound 30 | 3.22 | 5.19 | 196 | Blue |
| Example 1-31 | Compound 31 | 3.30 | 5.17 | 182 | Blue |
| Example 1-32 | Compound 32 | 3.33 | 5.25 | 168 | Blue |
| Example 1-33 | Compound 33 | 3.39 | 5.26 | 170 | Blue |
| Example 1-34 | Compound 34 | 3.40 | 5.30 | 166 | Blue |
| Example 1-35 | Compound 35 | 3.22 | 5.16 | 193 | Blue |
| Example 1-36 | Compound 36 | 3.30 | 5.06 | 184 | Blue |
| Example 1-37 | Compound 37 | 3.30 | 5.10 | 181 | Blue |
| Example 1-38 | Compound 38 | 3.50 | 5.34 | 198 | Blue |
| Example 1-39 | Compound 39 | 3.50 | 5.29 | 188 | Blue |
| Example 1-40 | Compound 40 | 3.50 | 5.36 | 180 | Blue |
| Example 1-41 | Compound 41 | 3.41 | 5.29 | 171 | Blue |
| Example 1-42 | Compound 42 | 3.41 | 5.28 | 166 | Blue |
| Example 1-43 | Compound 43 | 3.38 | 5.25 | 172 | Blue |
| Example 1-44 | Compound 44 | 3.28 | 5.18 | 191 | Blue |
| Example 1-45 | Compound 45 | 3.25 | 5.17 | 179 | Blue |
| Example 1-46 | Compound 46 | 3.24 | 5.17 | 198 | Blue |
| Example 1-47 | Compound 47 | 3.23 | 5.06 | 186 | Blue |
| Example 1-48 | Compound 48 | 3.28 | 5.18 | 197 | Blue |
| Example 1-49 | Compound 49 | 3.33 | 5.29 | 162 | Blue |
| Example 1-50 | Compound 50 | 3.38 | 5.32 | 163 | Blue |
| Example 1-51 | Compound 51 | 3.35 | 5.31 | 164 | Blue |
| Example 1-52 | Compound 52 | 3.48 | 5.24 | 182 | Blue |
| Example 1-53 | Compound 53 | 3.46 | 5.27 | 192 | Blue |
| Example 1-54 | Compound 54 | 3.45 | 5.34 | 196 | Blue |
| Example 1-55 | Compound 55 | 3.44 | 5.36 | 179 | Blue |
| Example 1-56 | Compound 56 | 3.39 | 5.17 | 202 | Blue |
| Example 1-57 | Compound 57 | 3.36 | 5.08 | 205 | Blue |
| Example 1-58 | Compound 58 | 3.32 | 5.16 | 207 | Blue |
| Example 1-59 | Compound 59 | 3.38 | 5.15 | 210 | Blue |
| Example 1-60 | Compound 60 | 3.35 | 5.19 | 212 | Blue |
| Example 1-61 | Compound 61 | 3.20 | 5.24 | 217 | Blue |
| Example 1-62 | Compound 62 | 3.20 | 5.23 | 232 | Blue |
| Example 1-63 | Compound 63 | 3.28 | 5.27 | 225 | Blue |
| Example 1-64 | Compound 64 | 3.32 | 5.10 | 218 | Blue |
| Example 1-65 | Compound 65 | 3.40 | 5.14 | 220 | Blue |
| Example 1-66 | Compound 66 | 3.31 | 5.07 | 214 | Blue |
| Example 1-67 | Compound 67 | 3.32 | 5.06 | 228 | Blue |
| Example 1-68 | Compound 68 | 3.38 | 5.07 | 201 | Blue |
| Example 1-69 | Compound 69 | 3.35 | 5.06 | 206 | Blue |
| Example 1-70 | Compound 70 | 3.32 | 5.10 | 212 | Blue |
| Example 1-71 | Compound 71 | 3.20 | 5.28 | 228 | Blue |
| Example 1-72 | Compound 72 | 3.23 | 5.25 | 229 | Blue |
| Example 1-73 | Compound 73 | 3.30 | 5.37 | 222 | Blue |
| Example 1-74 | Compound 74 | 3.26 | 5.29 | 219 | Blue |
| Example 1-75 | Compound 75 | 3.27 | 5.26 | 234 | Blue |
| Example 1-76 | Compound 76 | 3.39 | 5.18 | 229 | Blue |
| Example 1-77 | Compound 77 | 3.37 | 5.06 | 205 | Blue |
| Example 1-78 | Compound 78 | 3.40 | 5.13 | 227 | Blue |
| Example 1-79 | Compound 79 | 3.37 | 5.07 | 226 | Blue |
| Example 1-80 | Compound 80 | 3.33 | 5.10 | 207 | Blue |
| Example 1-81 | Compound 81 | 3.22 | 5.26 | 225 | Blue |
| Example 1-82 | Compound 82 | 3.30 | 5.34 | 238 | Blue |
| Example 1-83 | Compound 83 | 3.23 | 5.21 | 220 | Blue |
| Example 1-84 | Compound 84 | 3.34 | 5.24 | 266 | Blue |
| Example 1-85 | Compound 85 | 3.34 | 5.23 | 267 | Blue |
| Example 1-86 | Compound 86 | 3.39 | 5.21 | 227 | Blue |
| Example 1-87 | Compound 87 | 3.40 | 5.21 | 239 | Blue |
| Example 1-88 | Compound 88 | 3.43 | 5.19 | 265 | Blue |
| Example 1-89 | Compound 89 | 3.45 | 5.13 | 271 | Blue |
| Example 1-90 | Compound 90 | 3.54 | 5.13 | 260 | Blue |
| Example 1-91 | Compound 91 | 3.22 | 5.16 | 270 | Blue |
| Example 1-92 | Compound 92 | 3.27 | 5.06 | 264 | Blue |
| Example 1-93 | Compound 93 | 3.33 | 5.37 | 266 | Blue |
| Example 1-94 | Compound 94 | 3.35 | 5.23 | 213 | Blue |
| Example 1-95 | Compound 95 | 3.35 | 5.37 | 265 | Blue |
| Example 1-96 | Compound 96 | 3.45 | 5.19 | 226 | Blue |
| Example 1-97 | Compound 97 | 3.52 | 5.13 | 270 | Blue |
| Example 1-98 | Compound 98 | 3.49 | 5.08 | 266 | Blue |
| Example 1-99 | Compound 99 | 3.43 | 5.19 | 221 | Blue |
| Example 1-100 | Compound 100 | 3.33 | 5.22 | 253 | Blue |
| Example 1-101 | Compound 101 | 3.34 | 5.38 | 257 | Blue |
| Example 1-102 | Compound 102 | 3.32 | 5.34 | 237 | Blue |
| Example 1-103 | Compound 103 | 3.53 | 5.11 | 272 | Blue |
| Example 1-104 | Compound 104 | 3.55 | 5.13 | 266 | Blue |
| Example 1-105 | Compound 105 | 3.48 | 5.06 | 241 | Blue |
| Example 1-106 | Compound 106 | 3.48 | 5.13 | 272 | Blue |
| Example 1-107 | Compound 107 | 3.37 | 5.32 | 246 | Blue |
| Example 1-108 | Compound 108 | 3.34 | 5.29 | 257 | Blue |
| Example 1-109 | Compound 109 | 3.26 | 5.15 | 261 | Blue |
| Comparative | C-1 | 3.88 | 4.82 | 82 | Blue |
| Example 1-1 | | | | | |
| Comparative | C-2 | 3.82 | 4.88 | 61 | Blue |
| Example 1-2 | | | | | |
| Comparative | C-3 | 3.61 | 4.97 | 142 | Blue |
| Example 1-3 | | | | | |
| Comparative | C-4 | 3.70 | 4.91 | 114 | Blue |
| Example 1-4 | | | | | |
| Comparative | C-5 | 3.75 | 4.92 | 132 | Blue |
| Example 1-5 | | | | | |
| Comparative | C-6 | 3.73 | 4.85 | 108 | Blue |
| Example 1-6 | | | | | |
| Comparative | C-7 | 3.63 | 4.97 | 138 | Blue |
| Example 1-7 | | | | | |
| Comparative | C-8 | 3.94 | 4.56 | 75 | Blue |
| Example 1-8 | | | | | |

Referring to Table 1, it was confirmed that the organic light emitting devices of Examples 1-1 to 1-109 in which the compound represented by Chemical Formula 1 was as a hole transport auxiliary layer material of a blue light emitting layer was lower in driving voltage and remarkably improved in efficiency and lifetime, as compared with Comparative Examples 1-1 to 1-8. In particular, it was confirmed that the lifetime characteristics of the organic light emitting device were further improved when a compound represented by Chemical Formula 1 substituted with deuterium was used. It is considered that the devices of Examples exhibit excellent characteristics in terms of driving voltage, efficiency and lifetime because the compound represented by Chemical Formula 1 contributes to the stability of excitons formed in the light emitting layer.

### DESCRIPTION OF SYMBOLS

| | |
|---|---|
| 1: substrate | 2: anode |
| 3: organic material layer | 4: cathode |
| 5: hole injection layer | 6: hole transport layer |
| 7: hole transport auxiliary layer | 8: light emitting layer |
| 9: hole blocking layer | 10: electron transport layer |
| 11: electron injection layer | 12: electron injection and transport layer |

1. A compound represented by the following Chemical Formula 1: 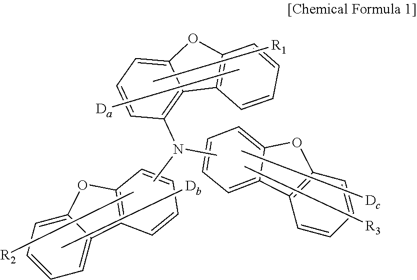
wherein, in Chemical Formula 1:
D is deuterium;
a to c are each independently an integer of 0 to 6;
R₁ to R₃ are each independently hydrogen, deuterium, or -Lₐ-Rₐ, with the proviso that at least one of R₁ to R₃ is -Lₐ-Rₐ;
Lₐ is a single bond or a substituted or unsubstituted C₆₋₃₀ arylene;
Rₐ is a substituted or unsubstituted C₆₋₃₀ aryl; a substituted or unsubstituted C₂₋₃₀ heteroaryl containing at least one heteroatom of O and S; a fused ring of a substituted or unsubstituted C₃₋₁₂ aliphatic ring and a C₆₋₃₀ aryl; or a substituted or unsubstituted C₆₋₃₀ arylsilyl,
with the proviso that the case where all of R₁ to R₃ are unsubstituted phenyl is excluded.

2. The compound as claimed in claim 1, wherein:
Rₐ is substituted with at least one deuterium and/or at least one C₁₋₁₀ alkyl.

3. The compound as claimed in claim 1, wherein:
Lₐ is a single bond; phenylene; biphenyldiyl; or naphthalenediyl.

4. The compound as claimed in claim 1, wherein:
Rₐ is phenyl; biphenylyl; terphenylyl; naphthyl; (phenyl)naphthyl; phenanthrenyl; fluoranthenyl; benzo[a]phenanthrenyl; benzo[c]phenanthrenyl; dibenzofuran; dibenzothiophene; triphenylsilyl; 2,3-dihydro-1H-indenyl; 1,2,3,4-tetrahydronaphthalenyl; or 6,7,8,9-tetrahydro-5H-benzo[7]annulene,
wherein the Rₐ is unsubstituted or substituted with at least one substituent selected from the group consisting of deuterium; methyl; ethyl; propyl; isopropyl; and t-butyl.

5. The compound as claimed in claim 1, wherein:
the compound represented by Chemical Formula 1 is any one compound selected from among the following compounds: 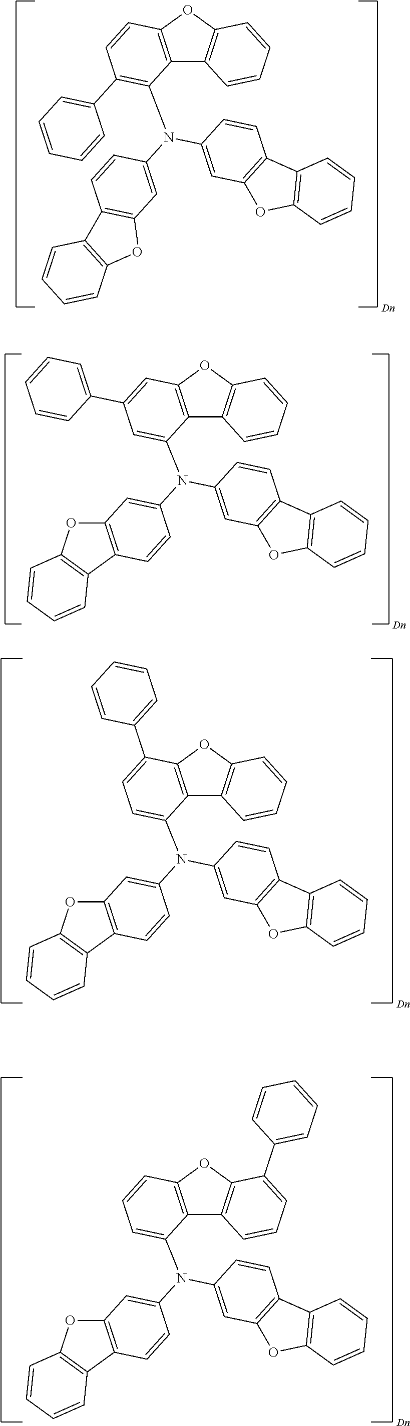 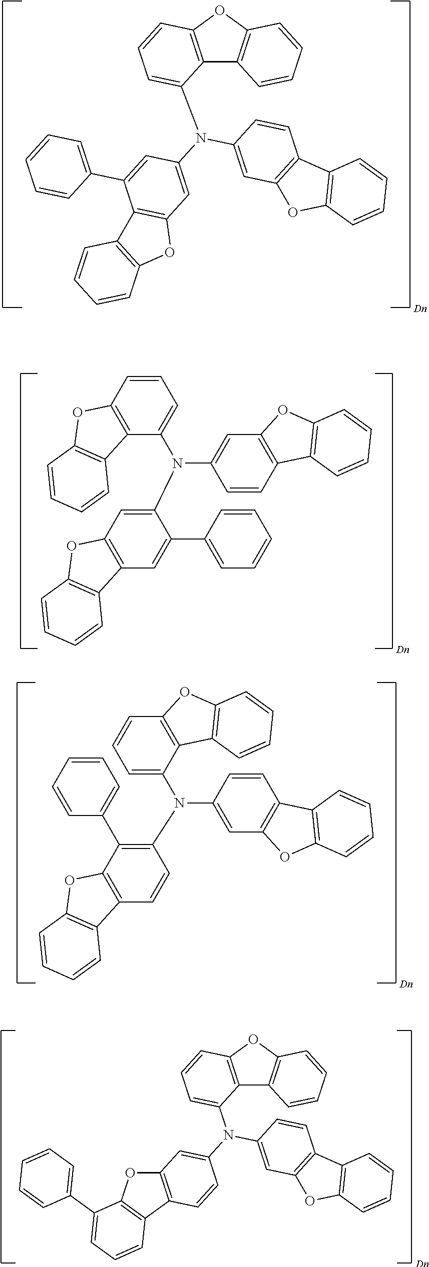 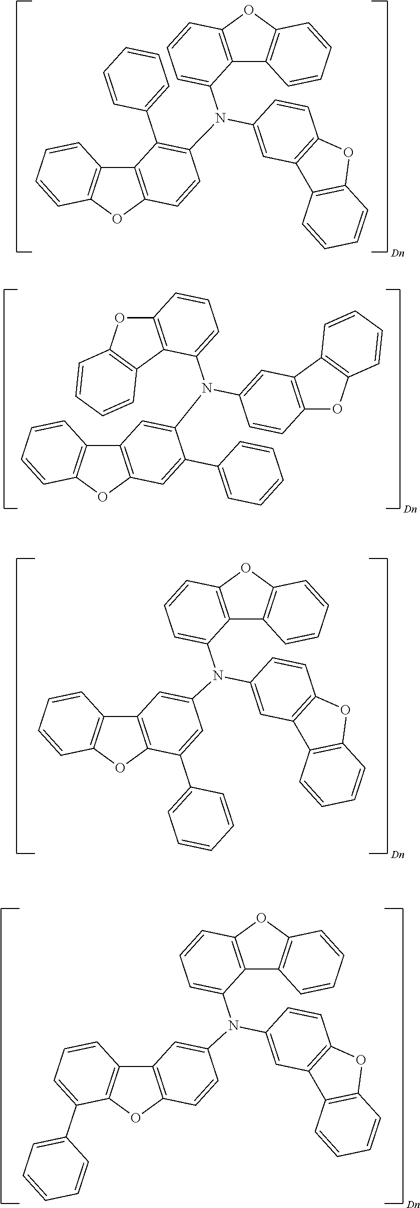 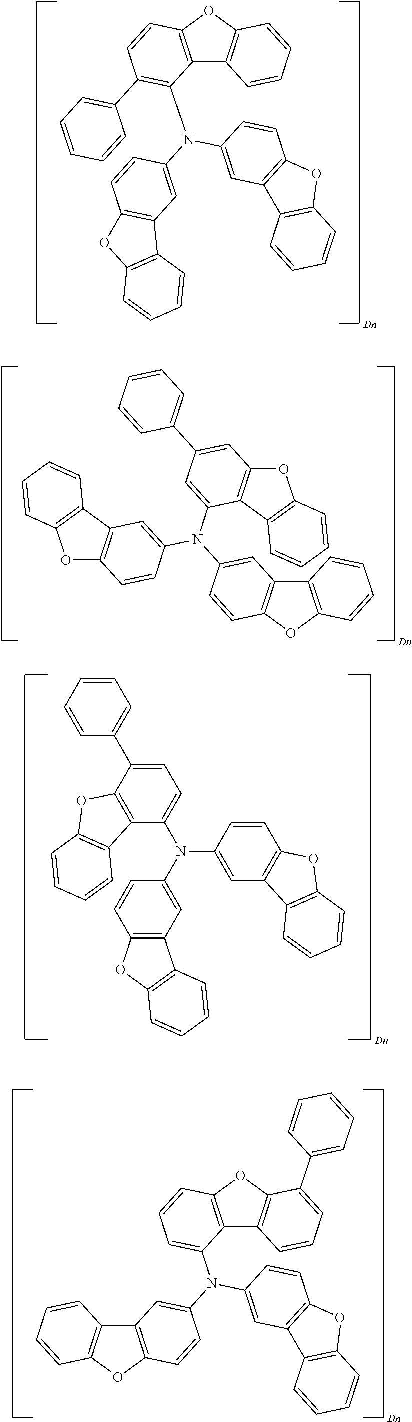 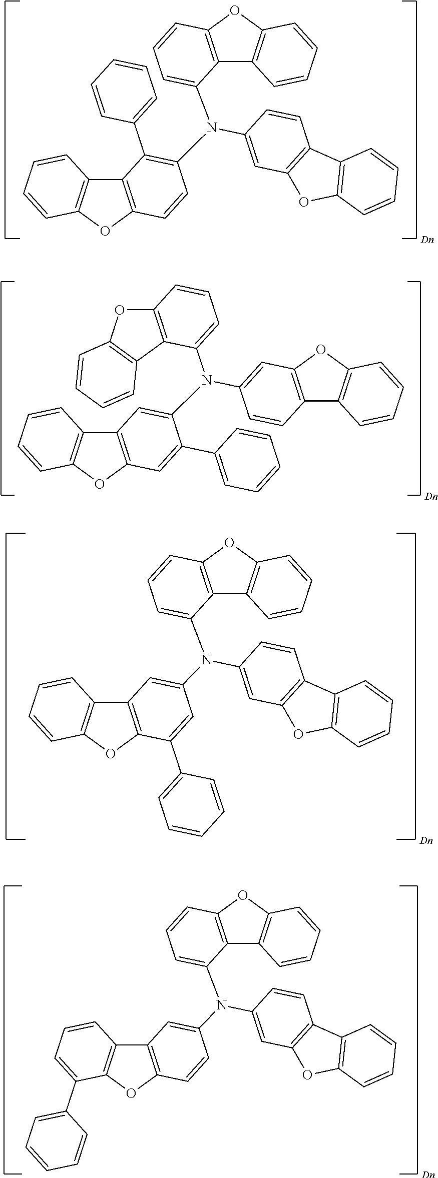 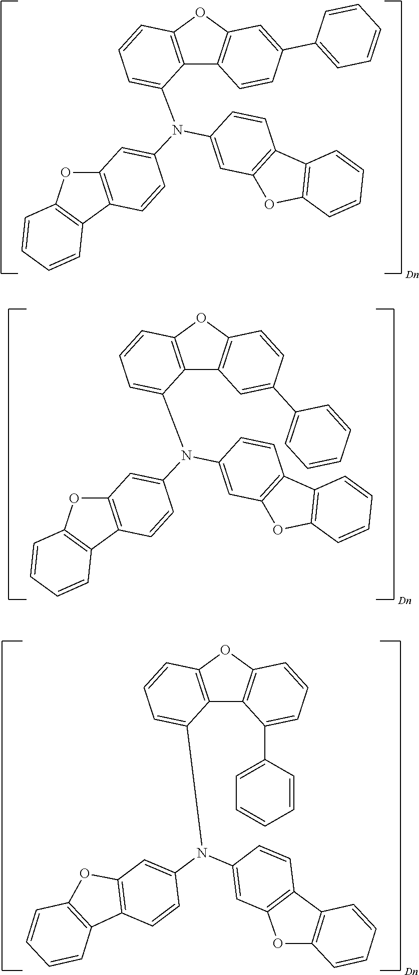 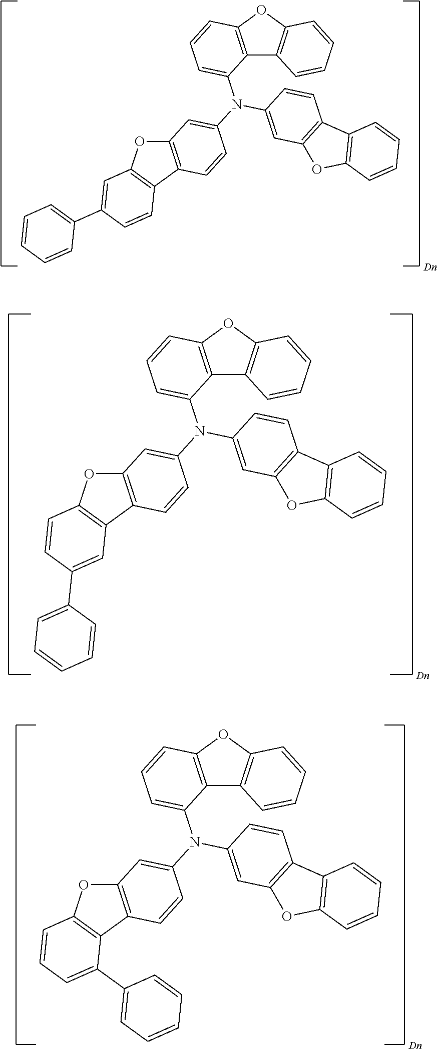 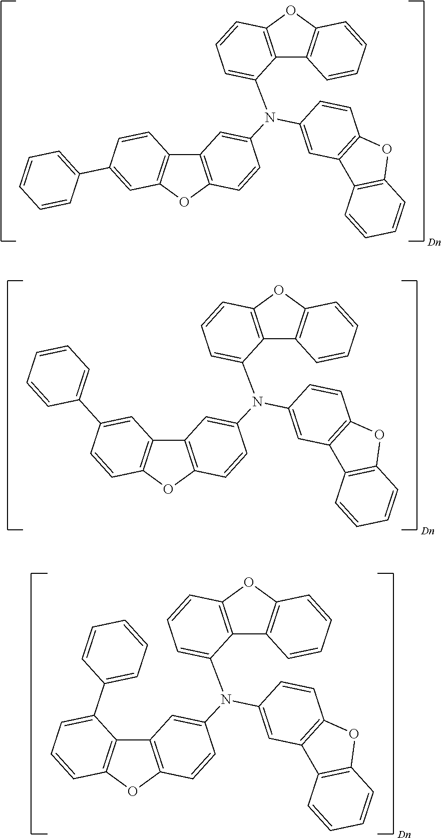 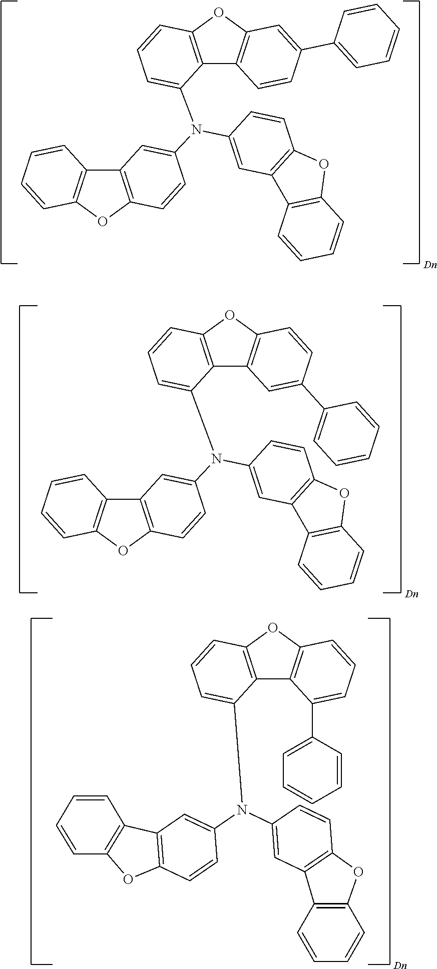 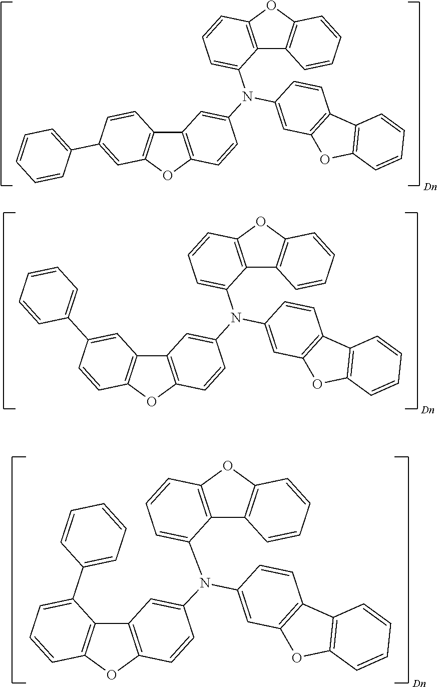 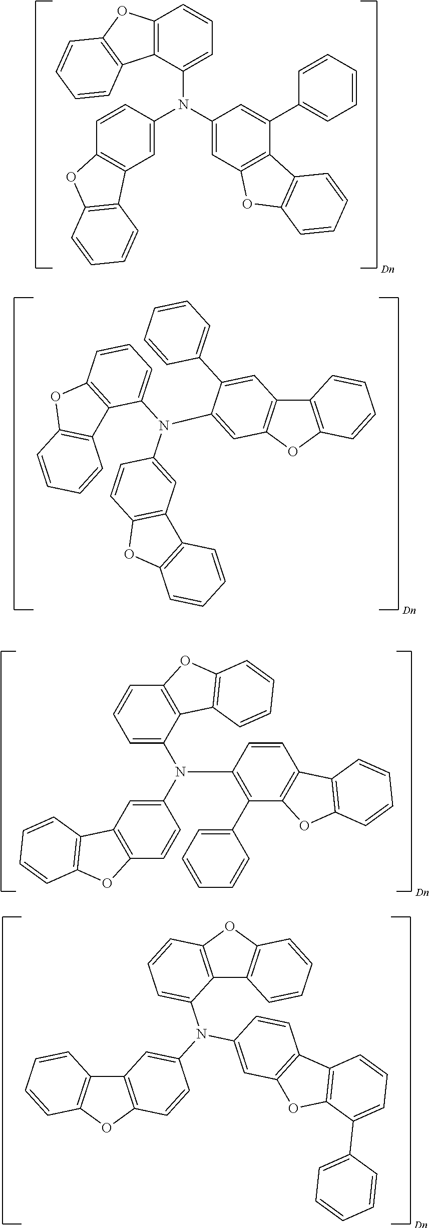 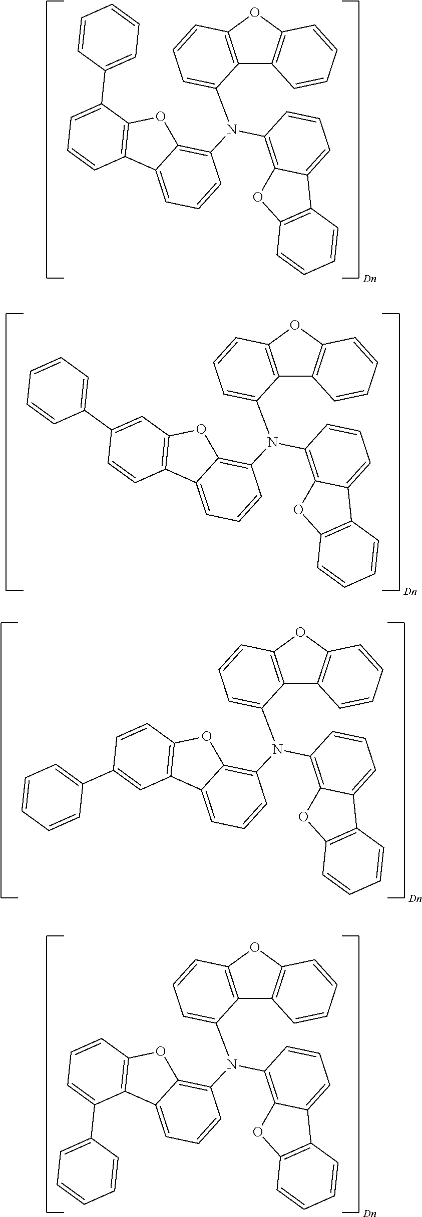 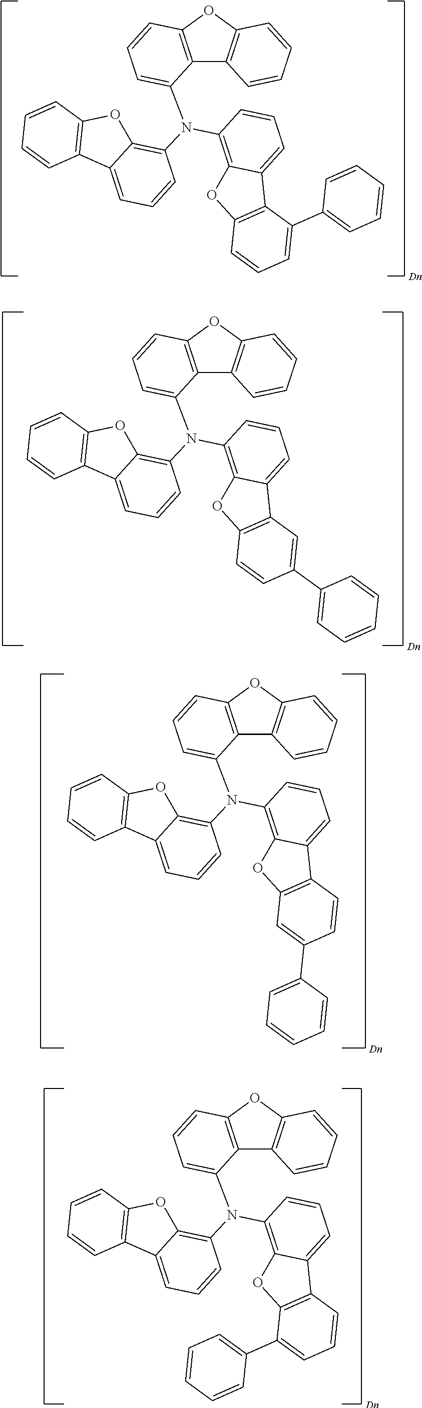 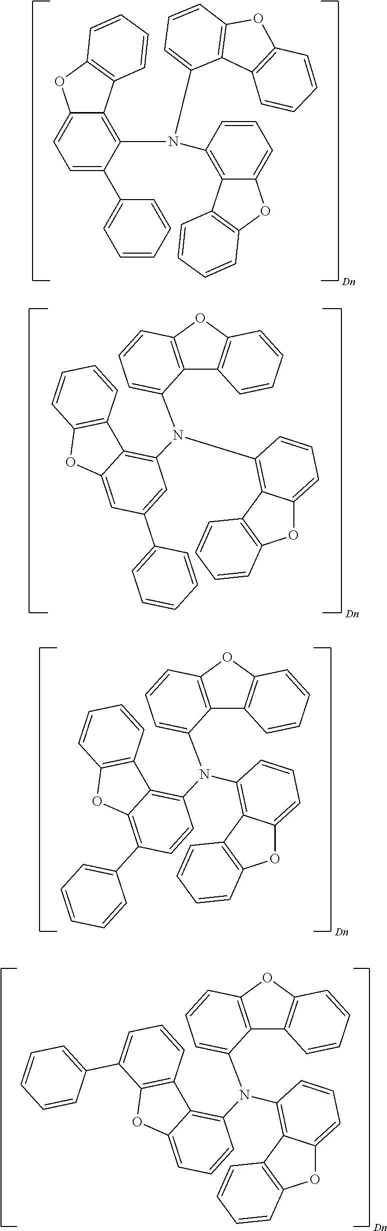 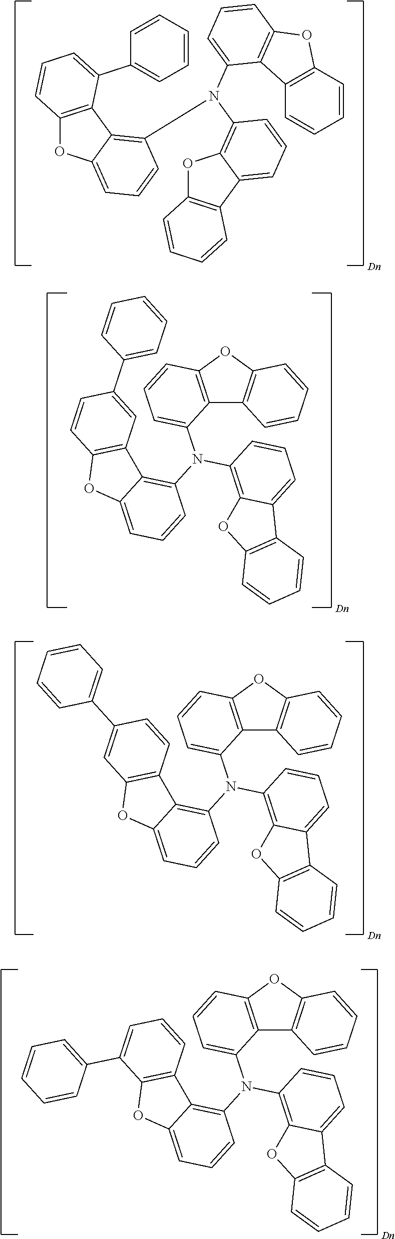 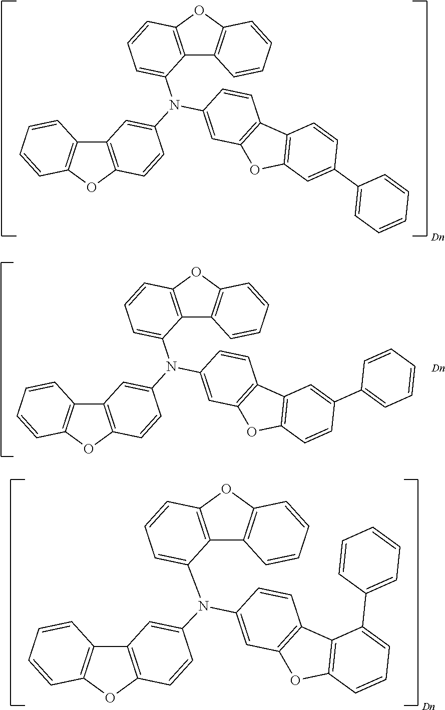 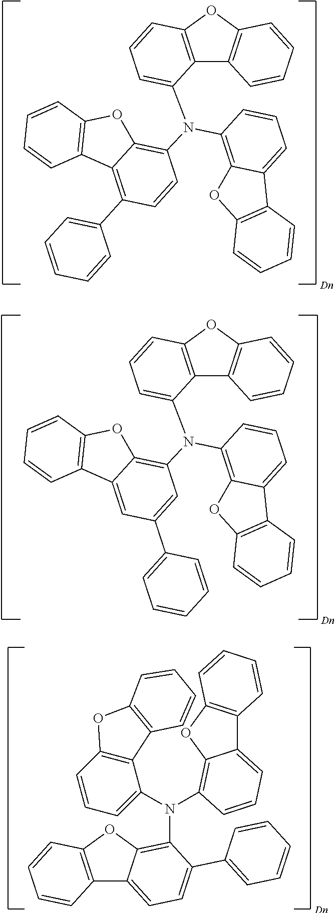 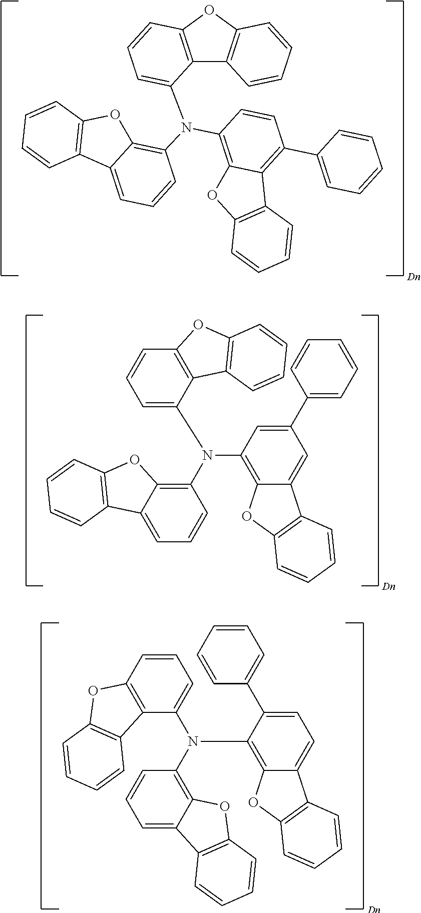 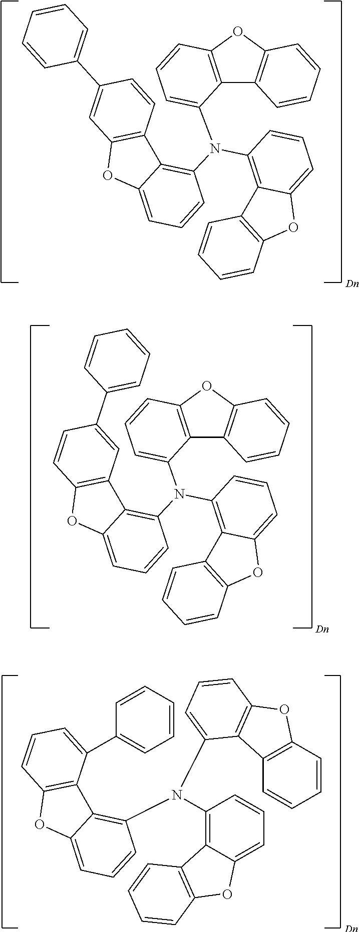 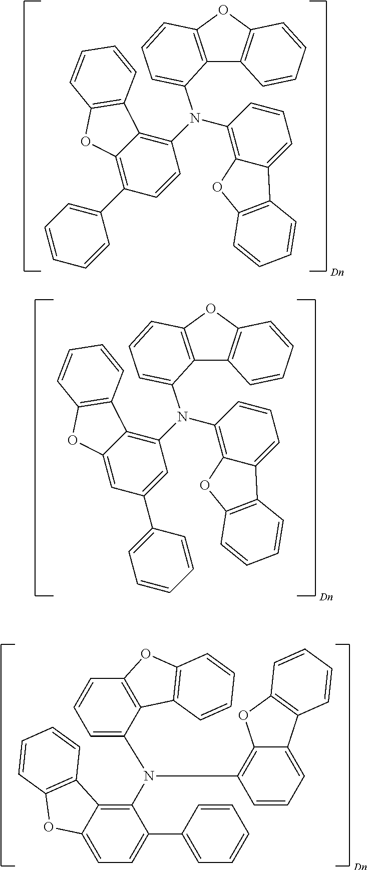 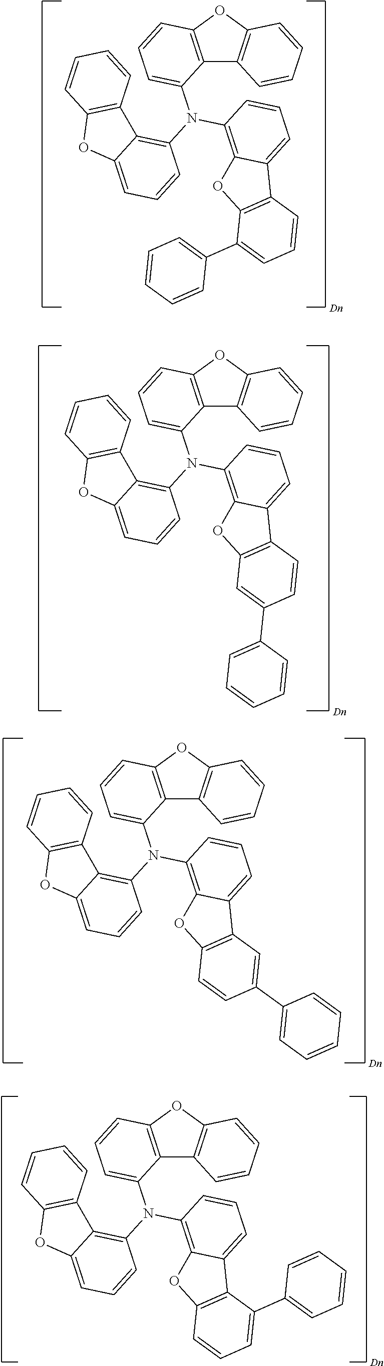 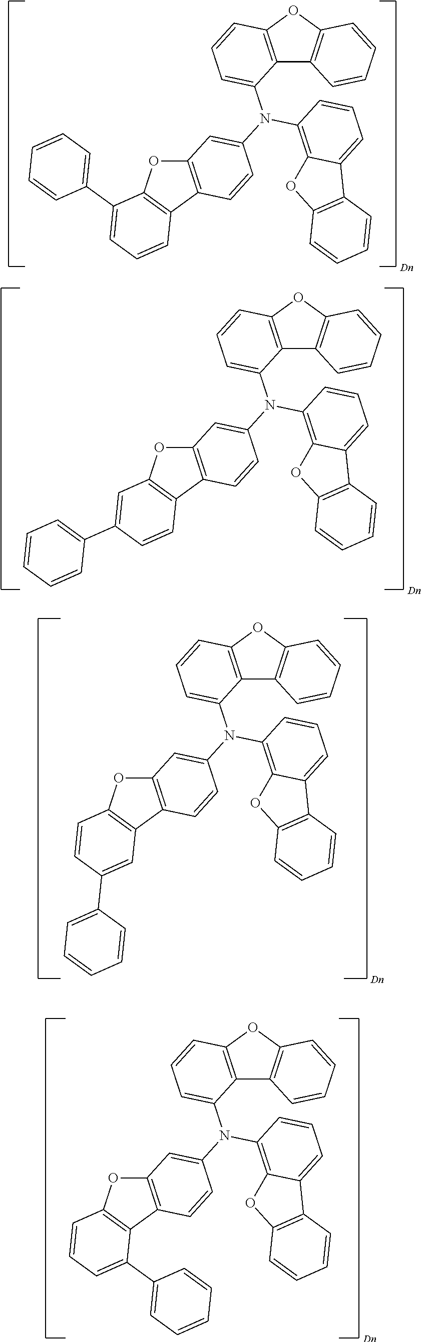 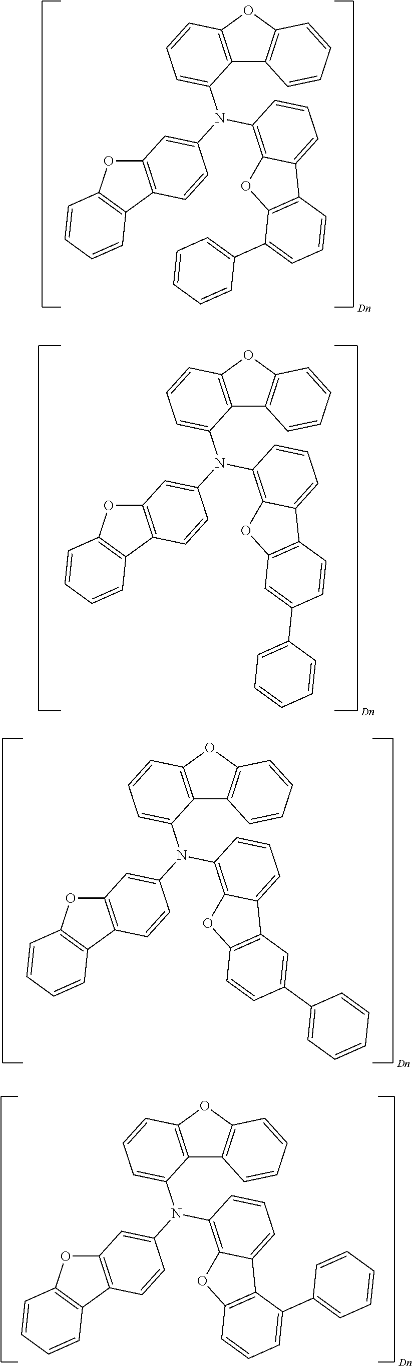 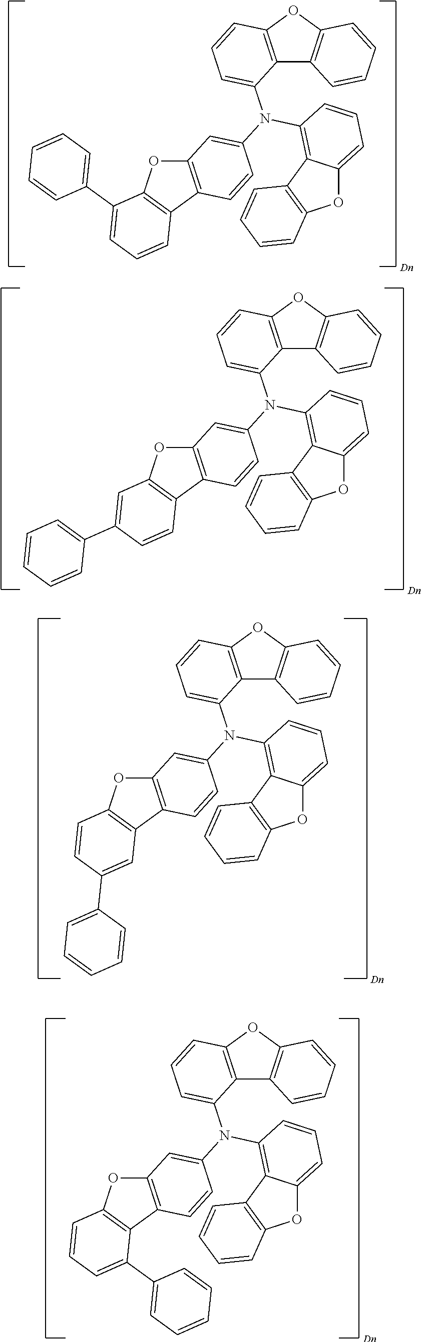 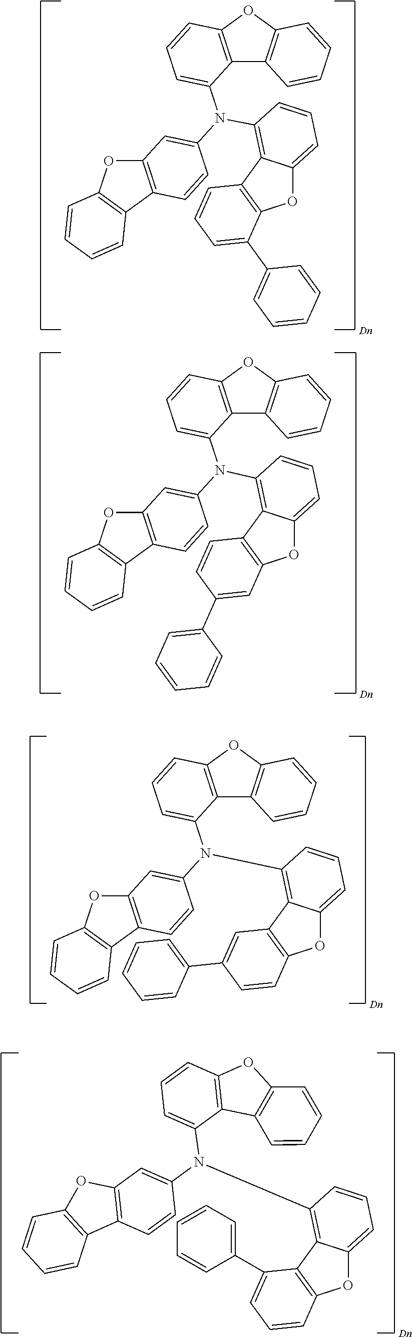 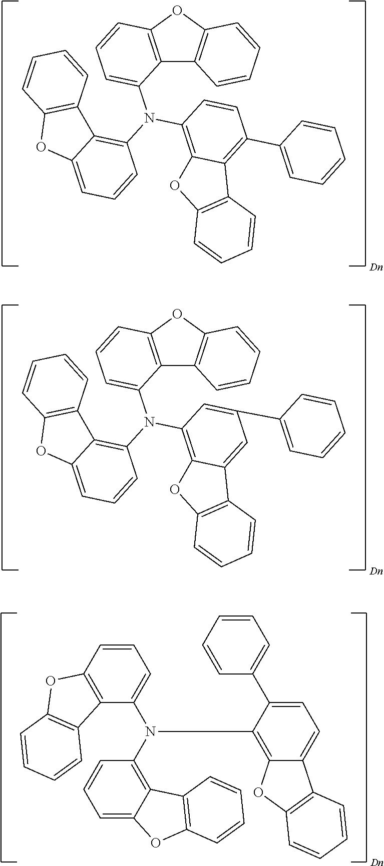 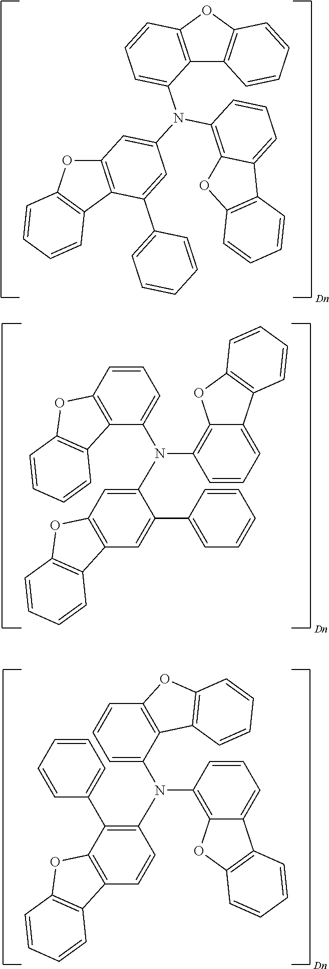 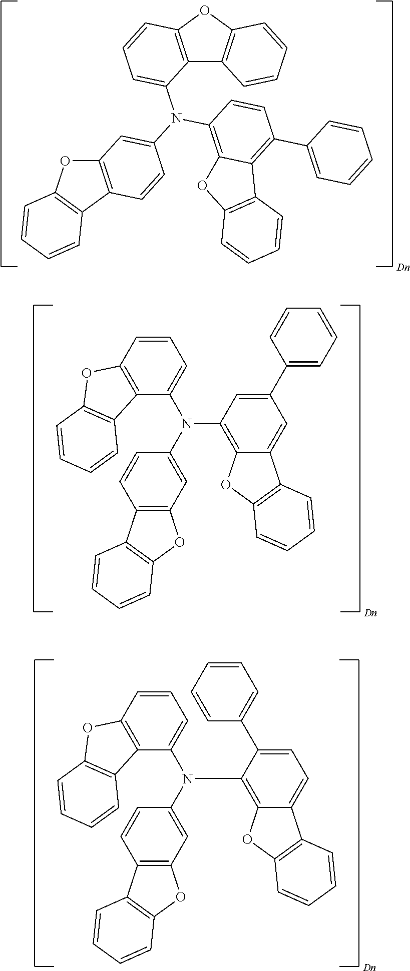 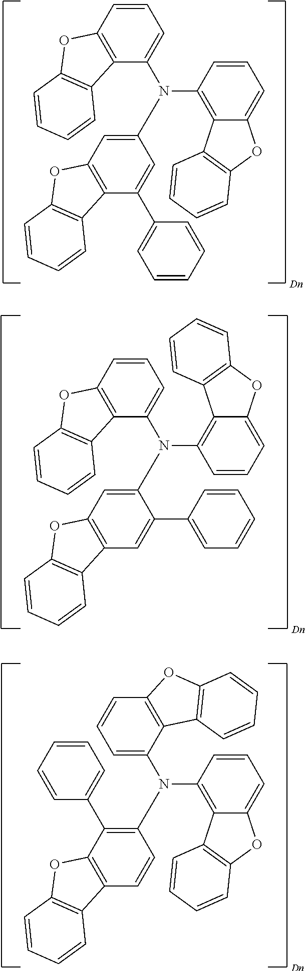 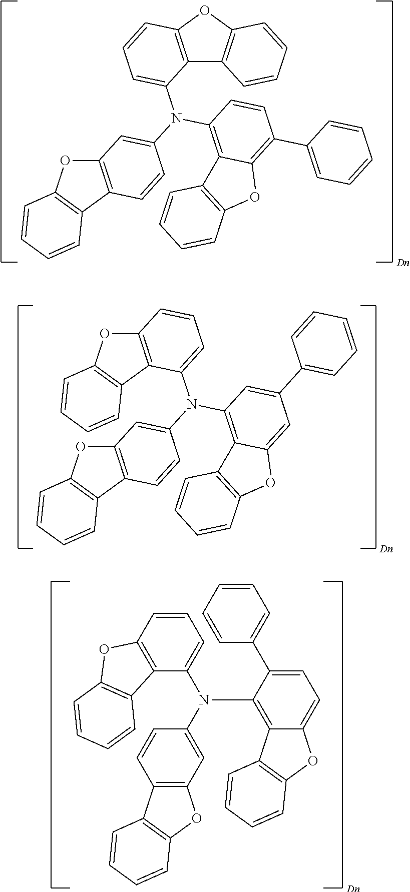 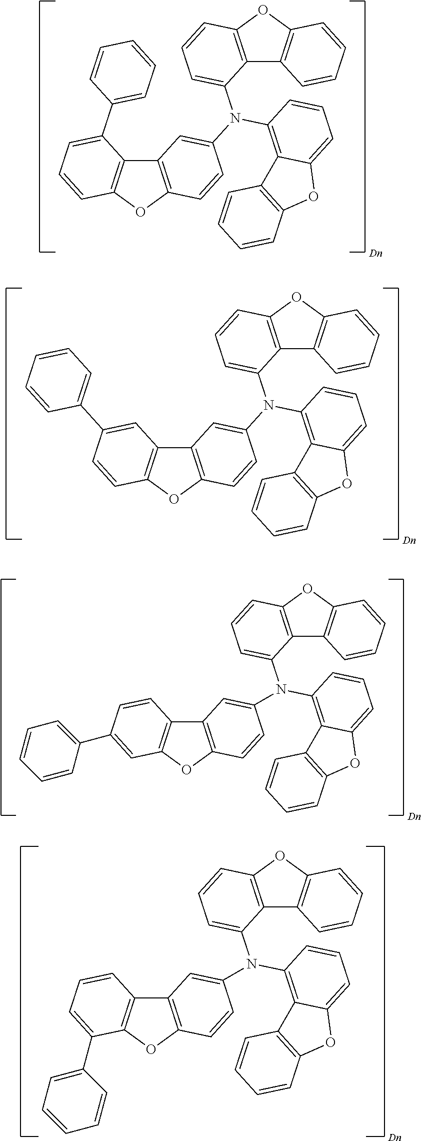 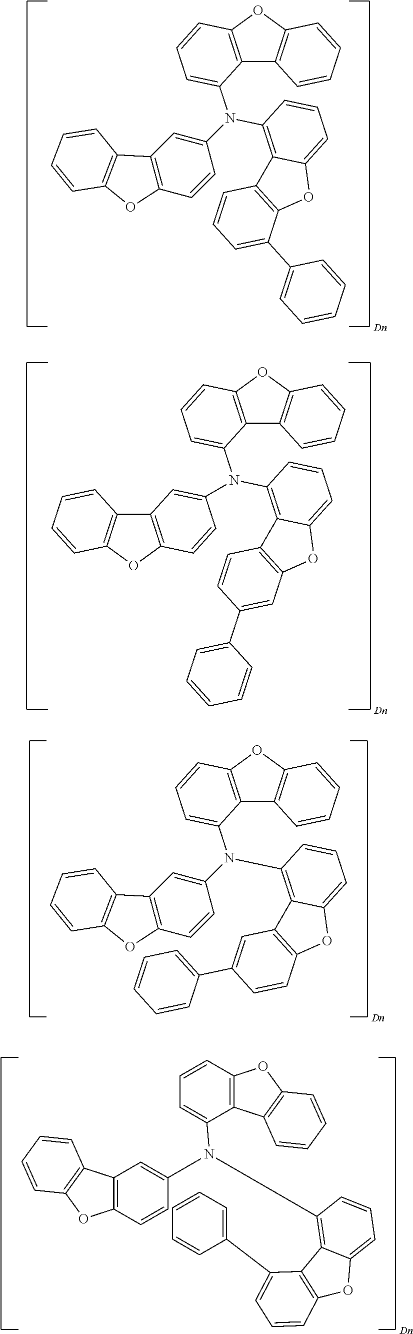 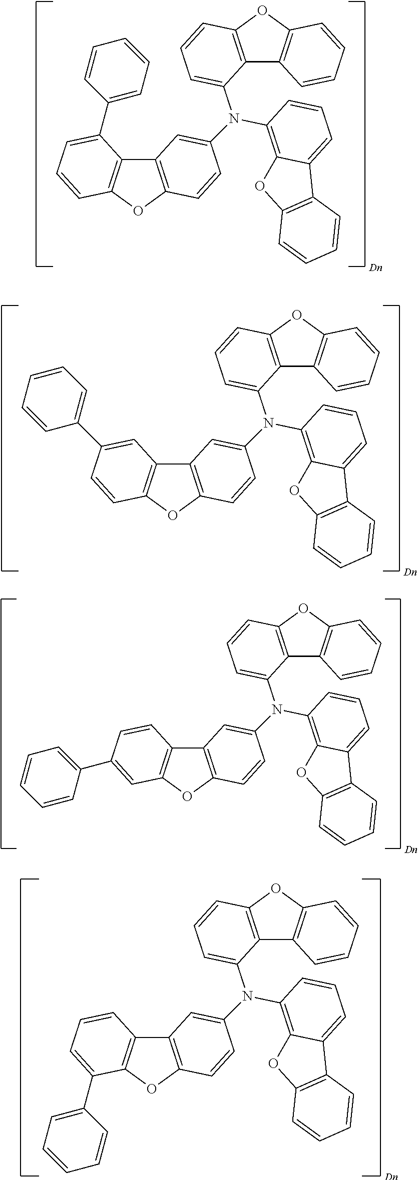 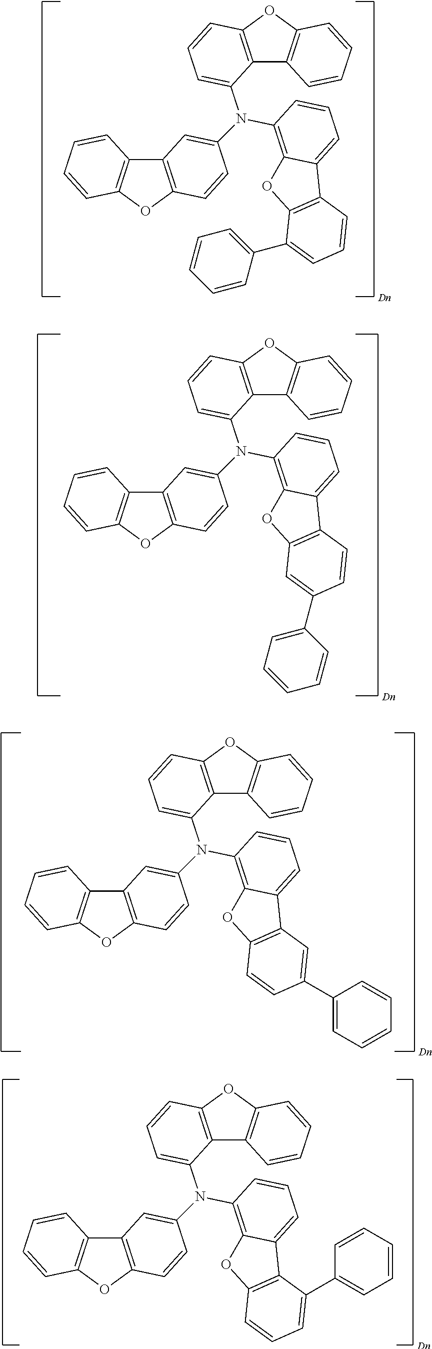 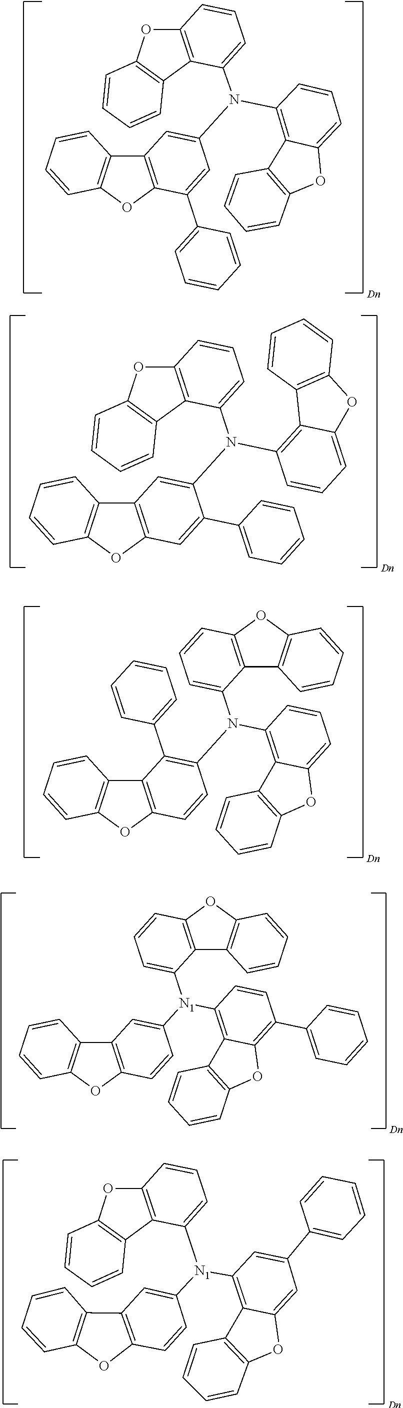 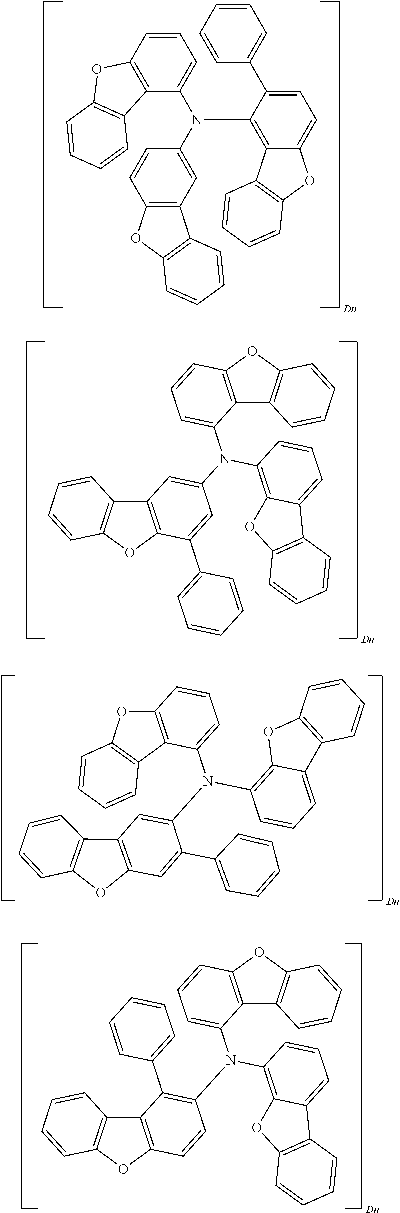 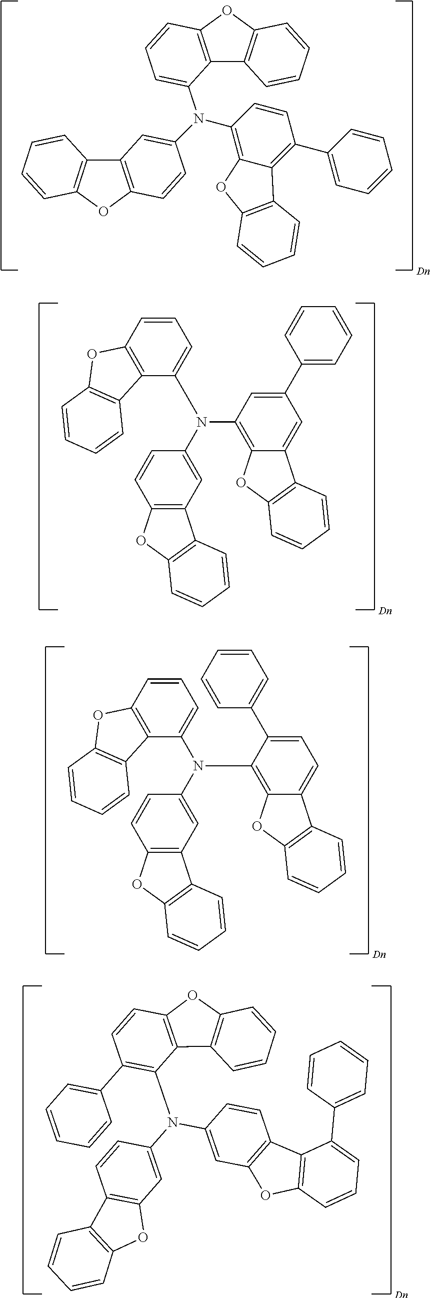 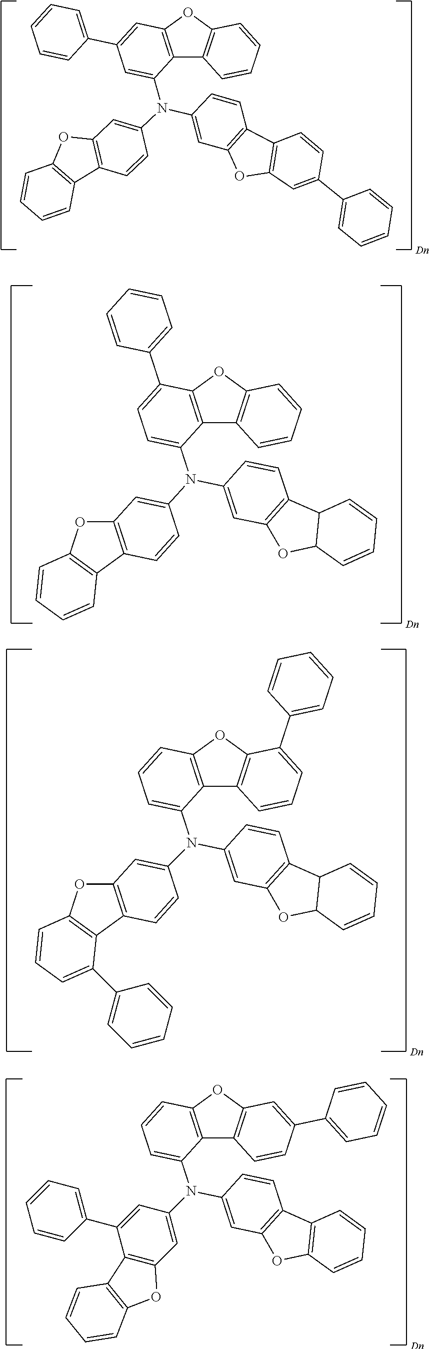 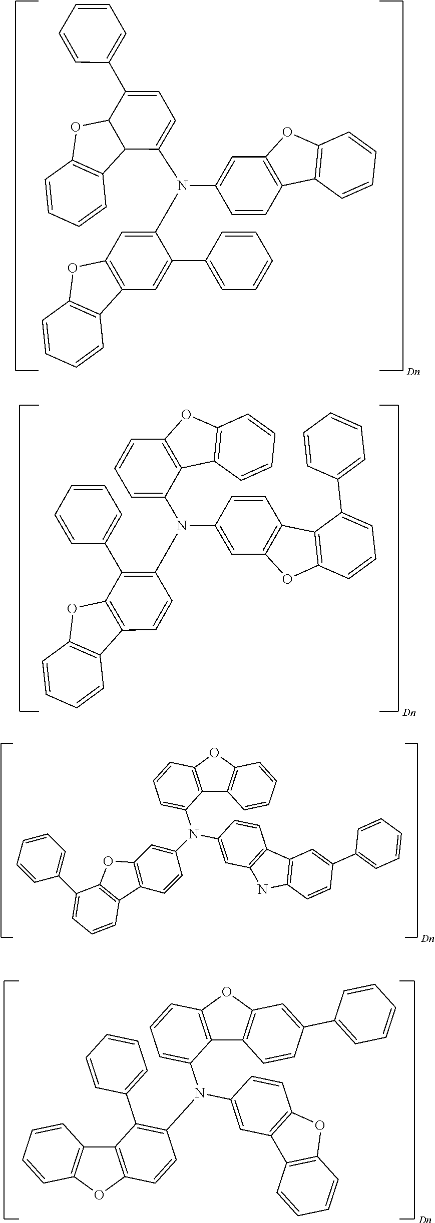 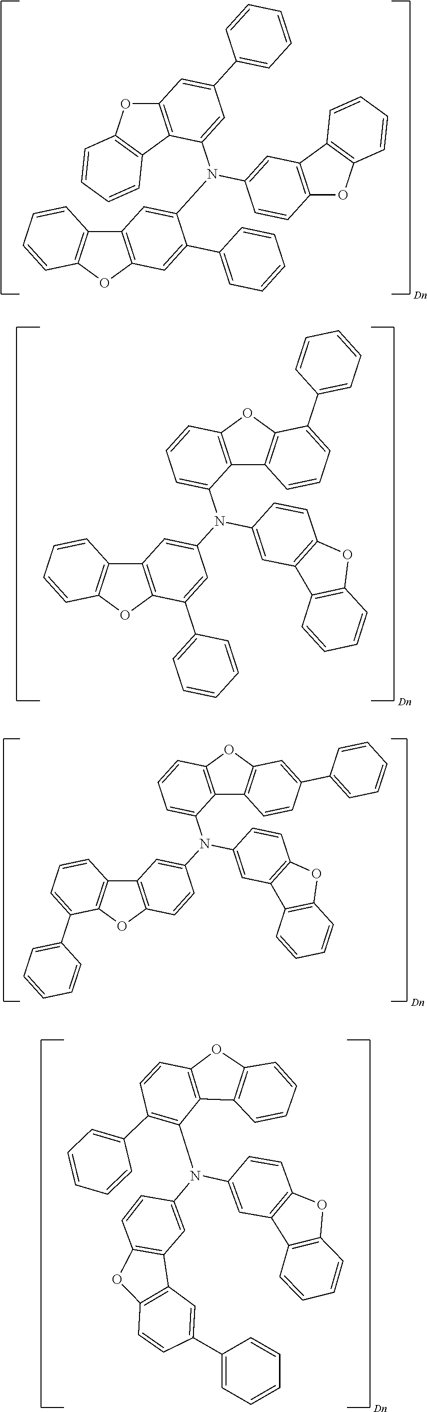 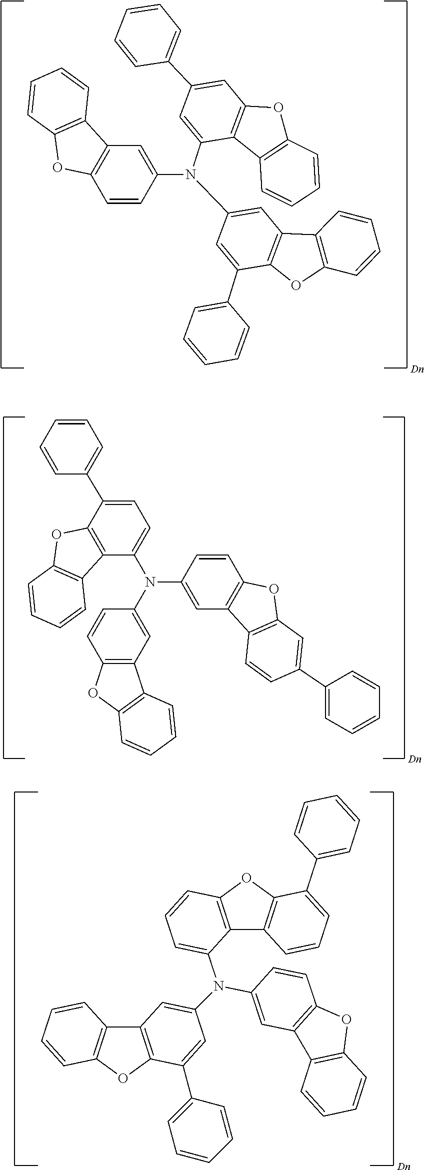 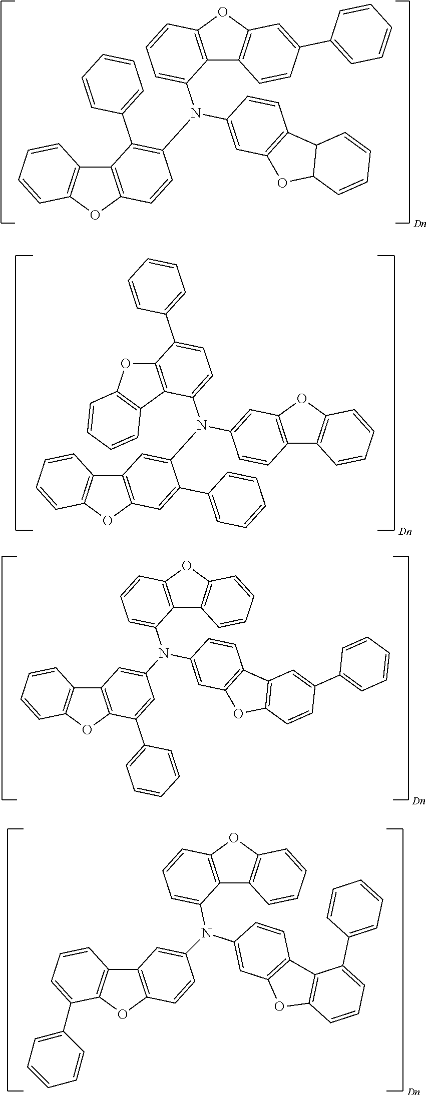 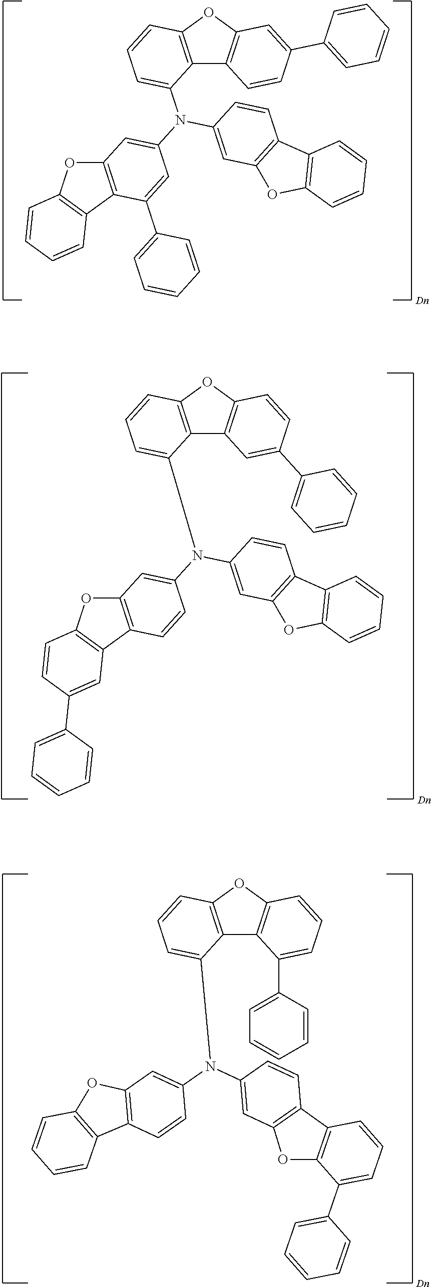 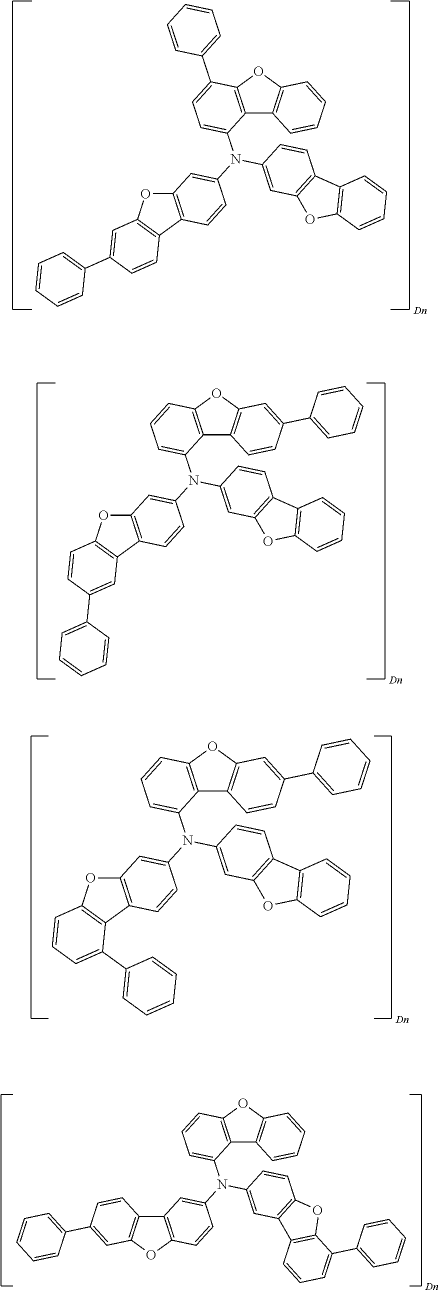 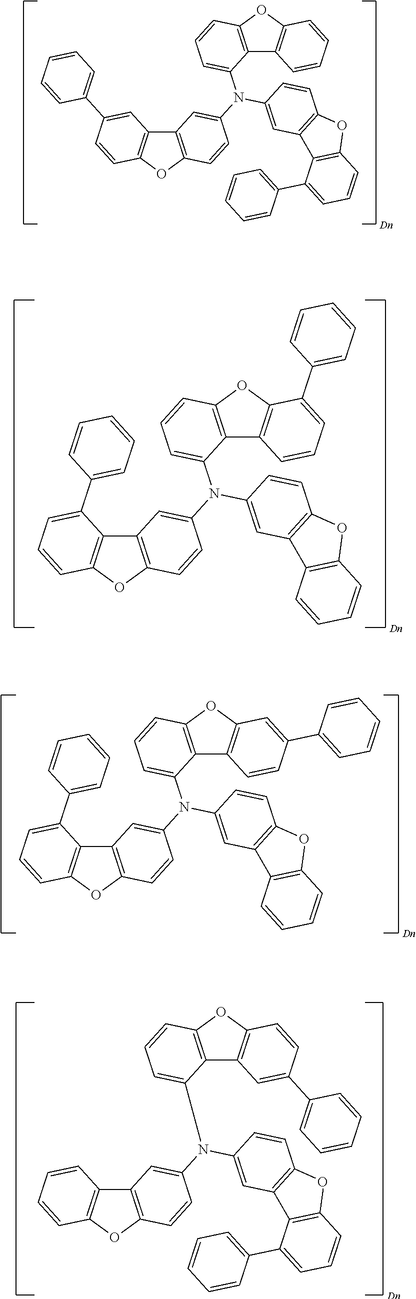 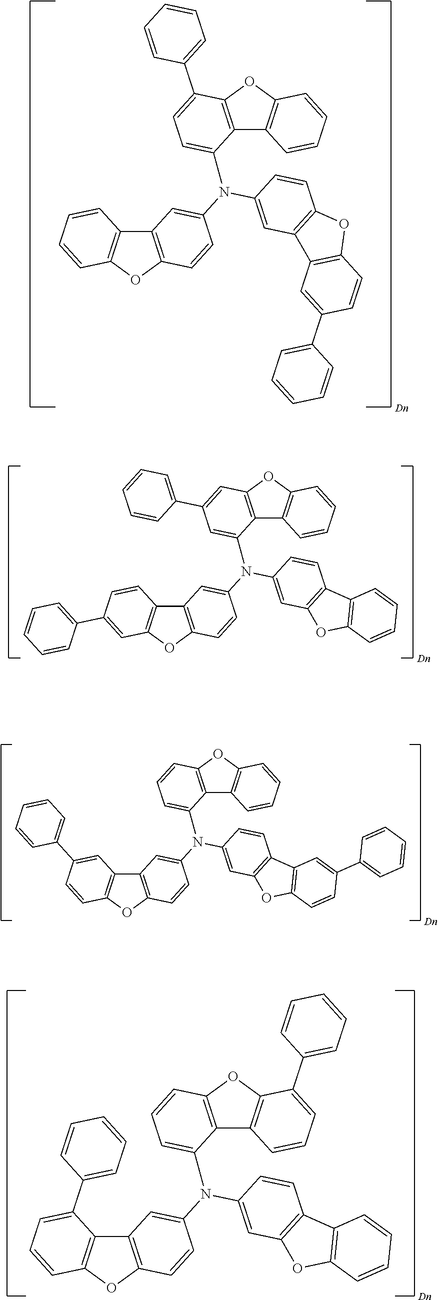 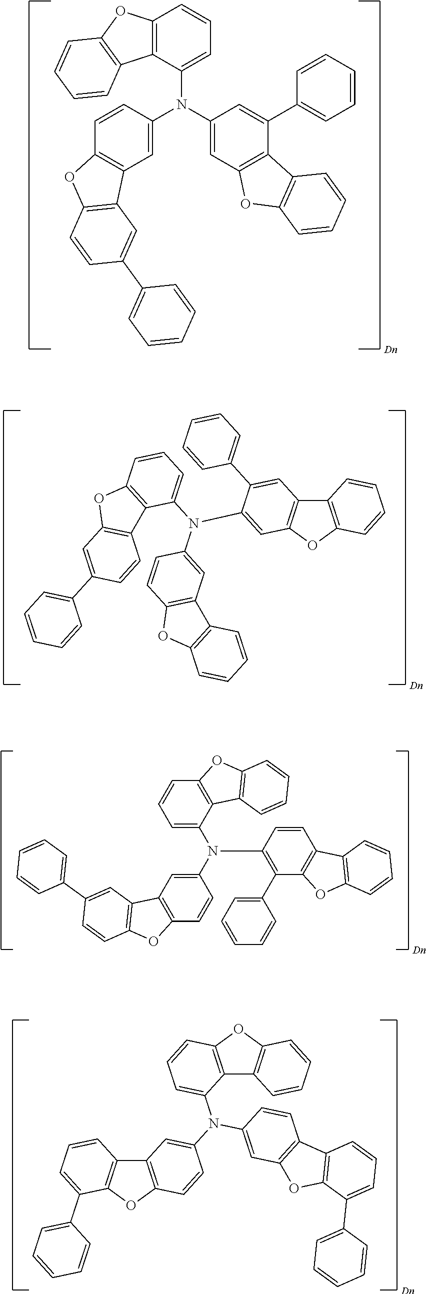 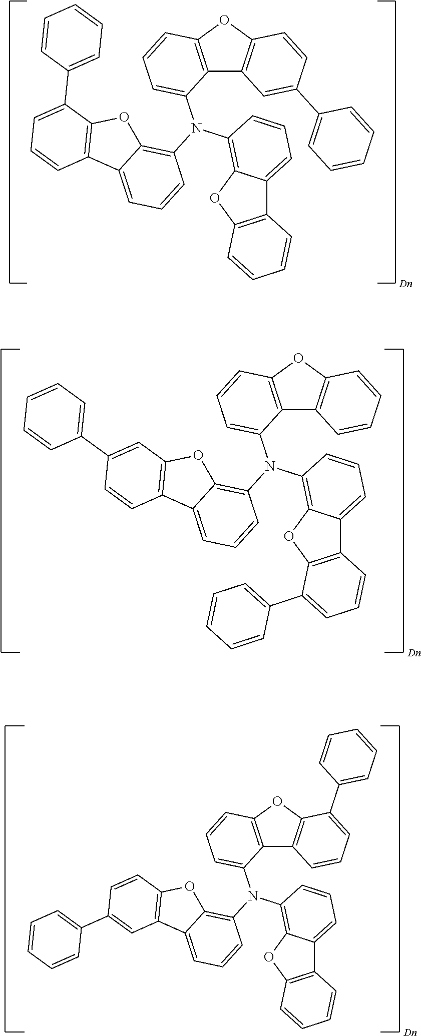 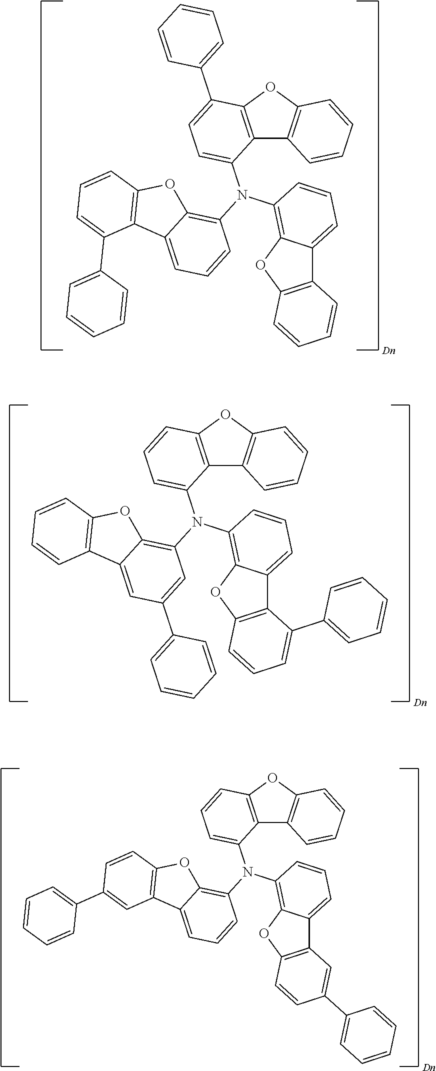 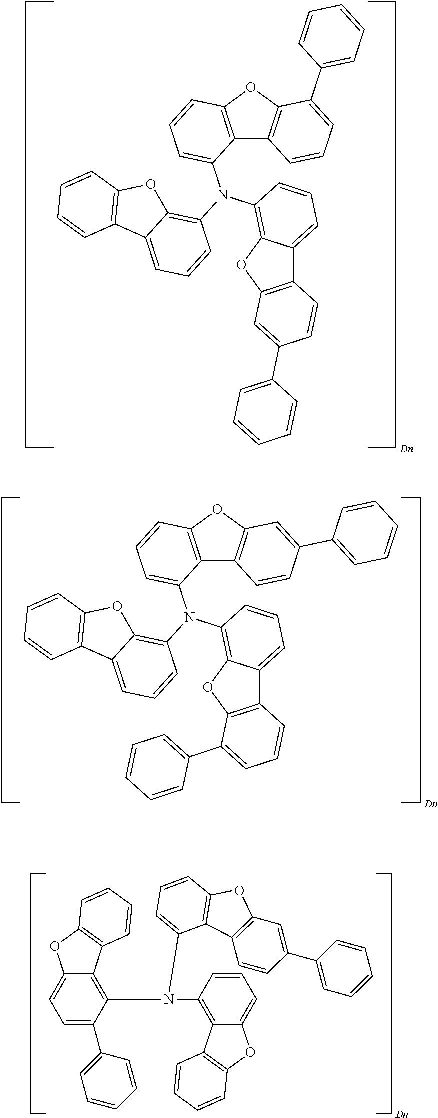 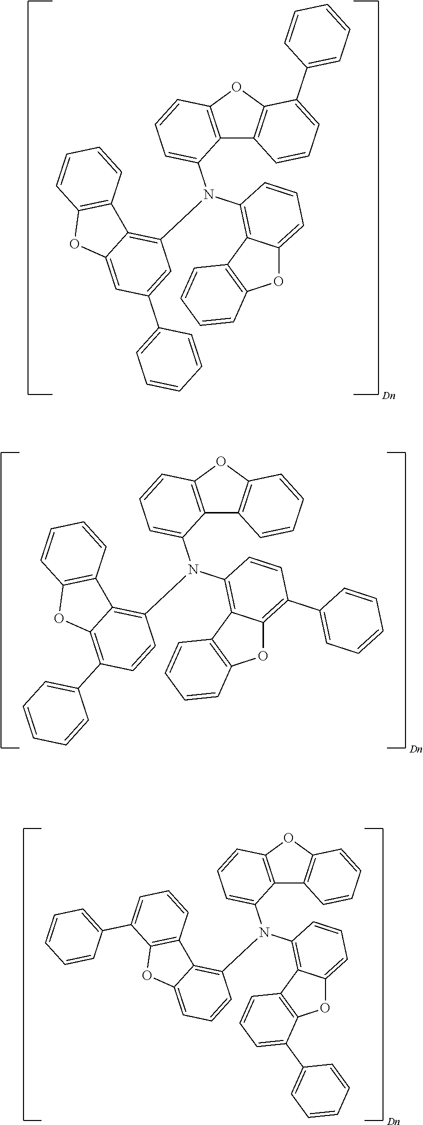 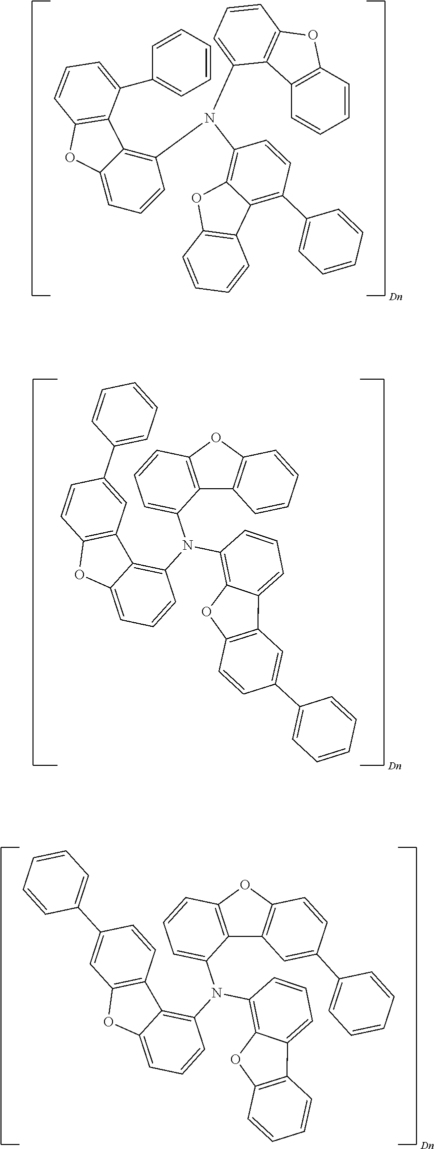 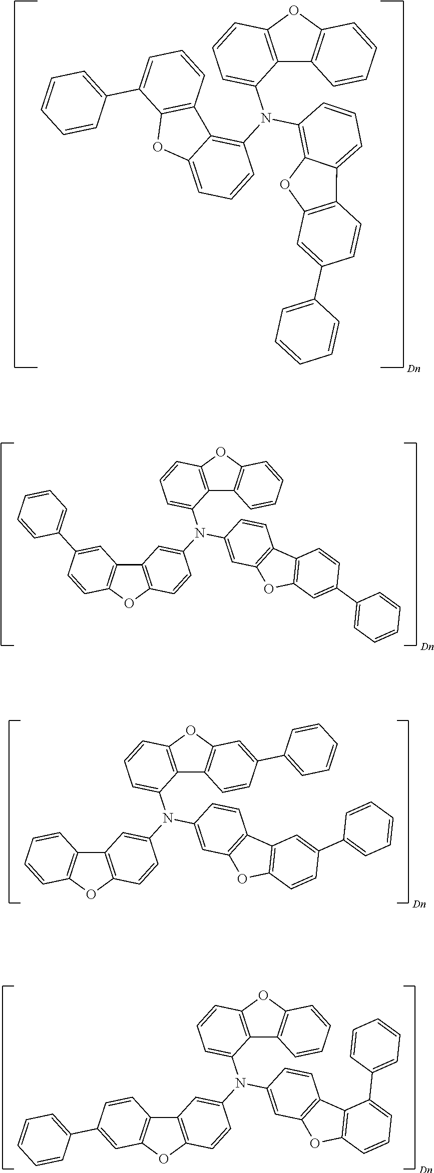 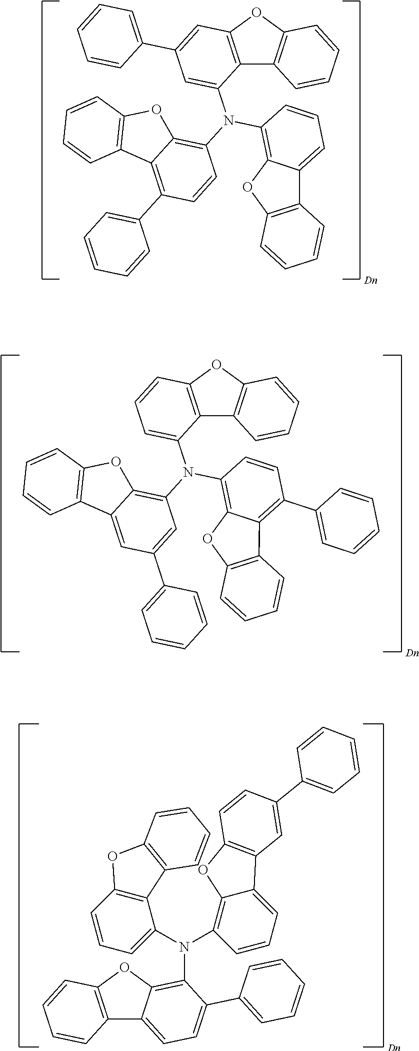 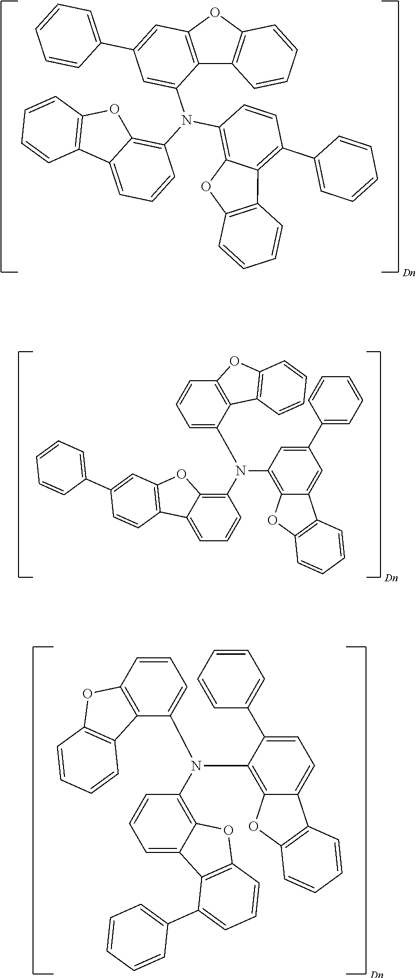 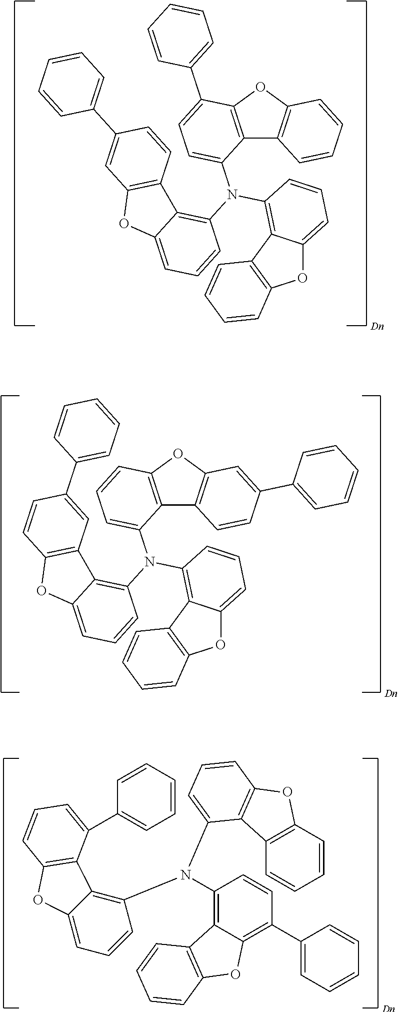 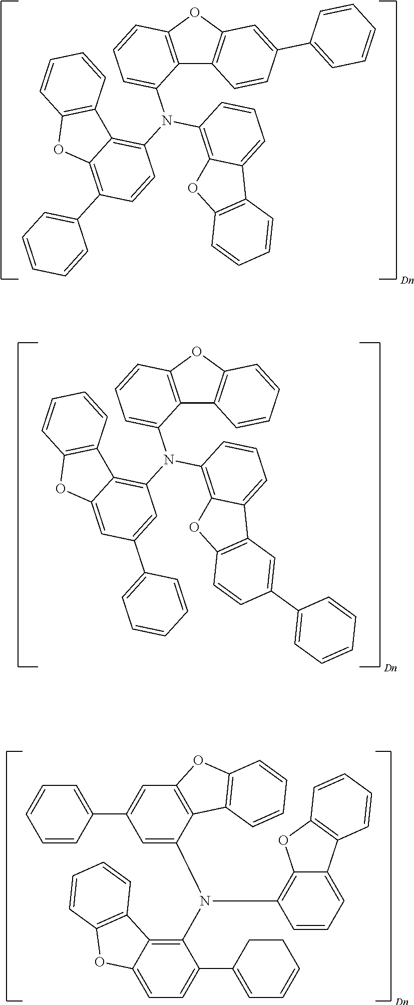 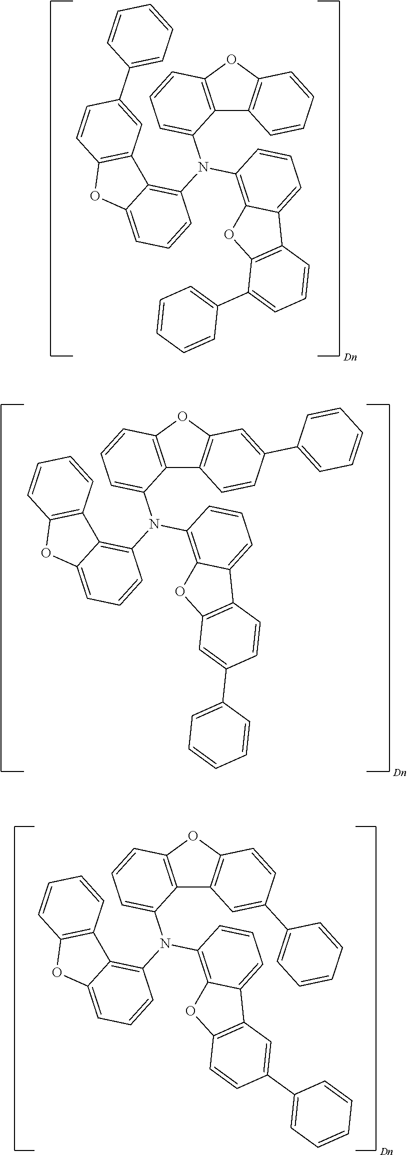 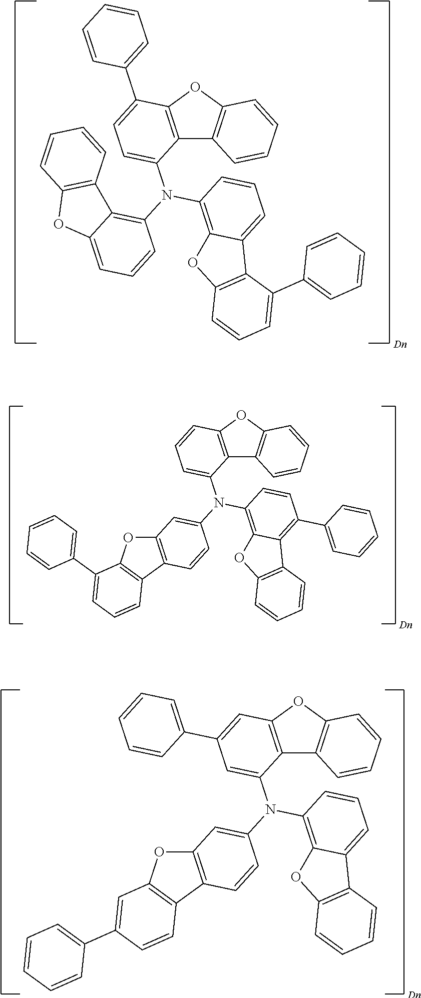 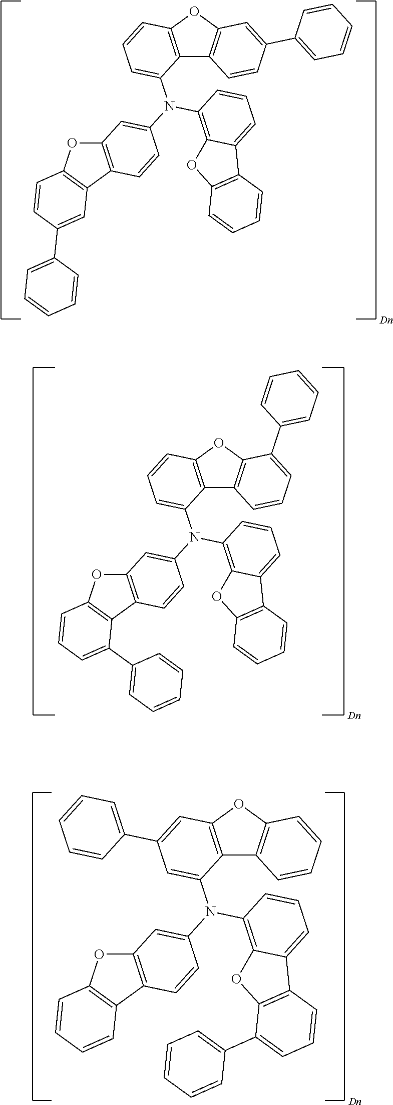 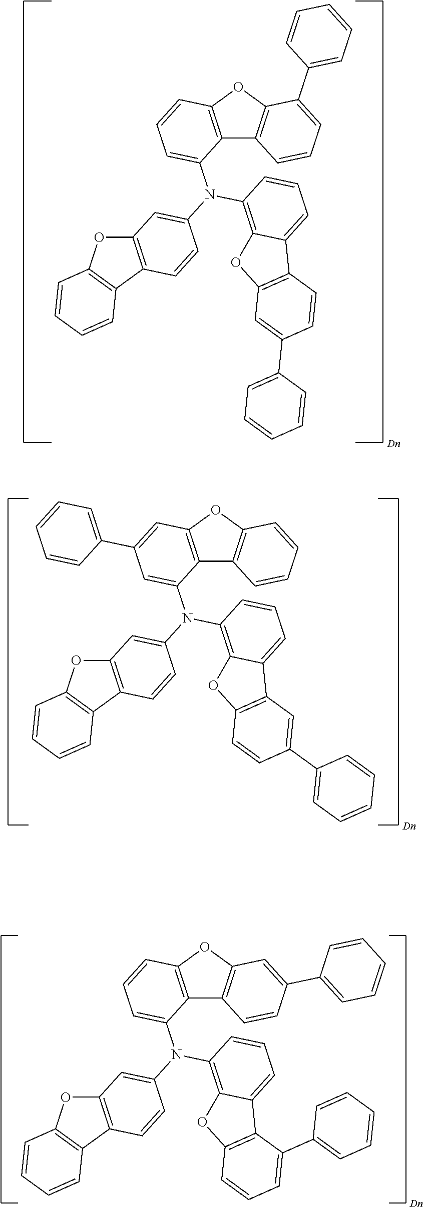 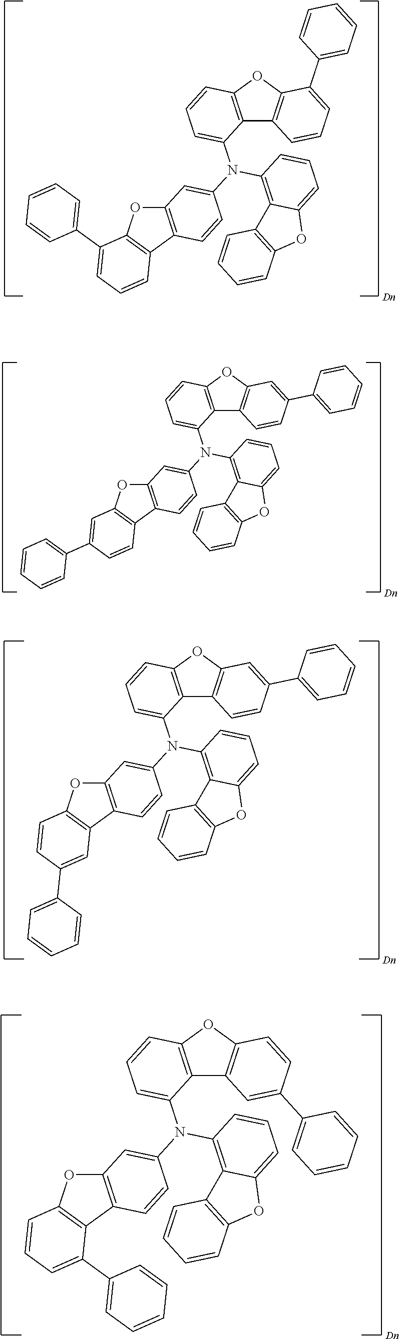 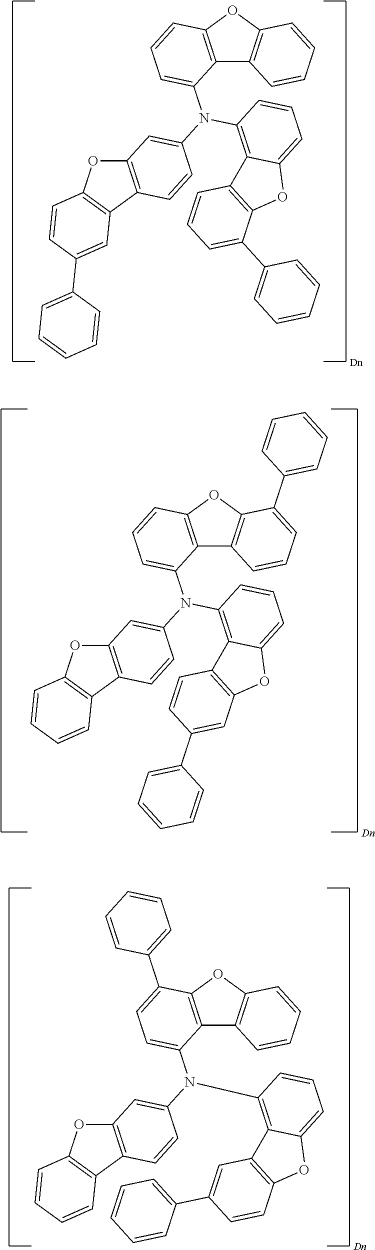 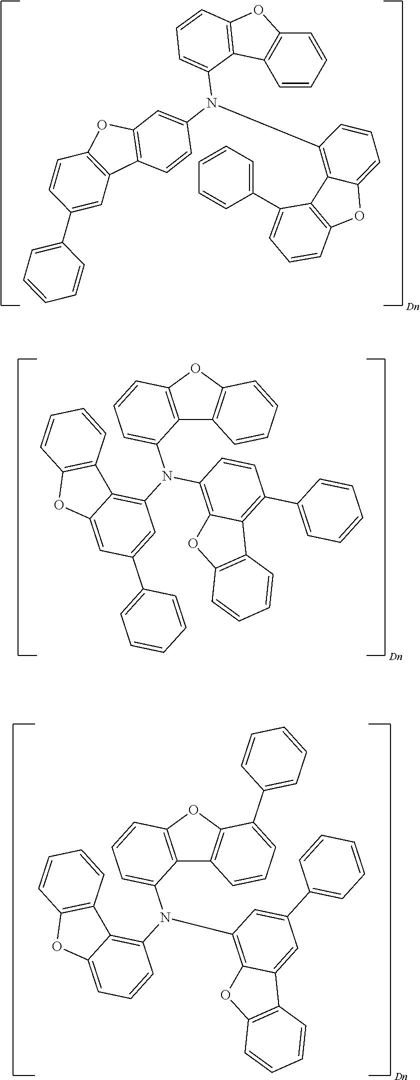 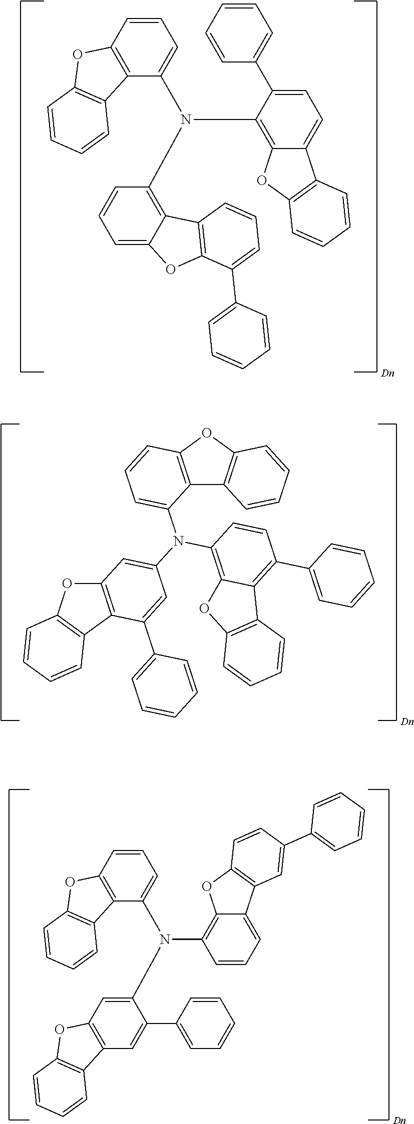 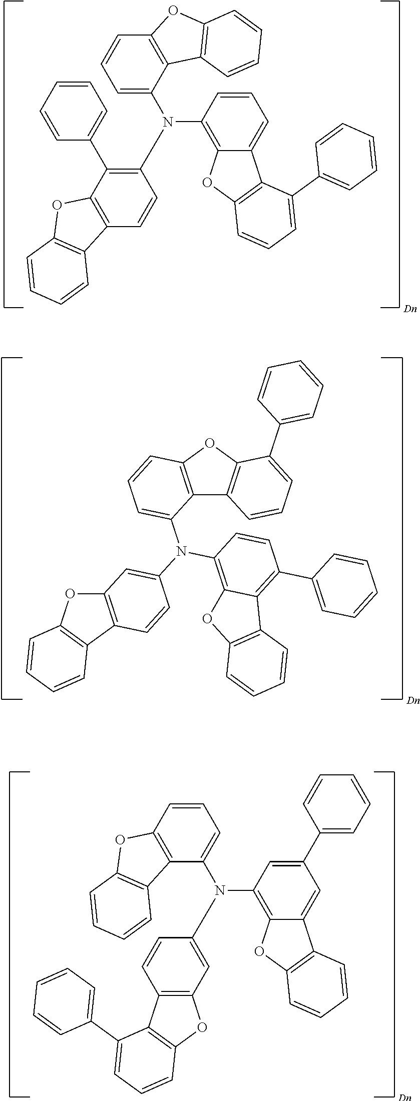 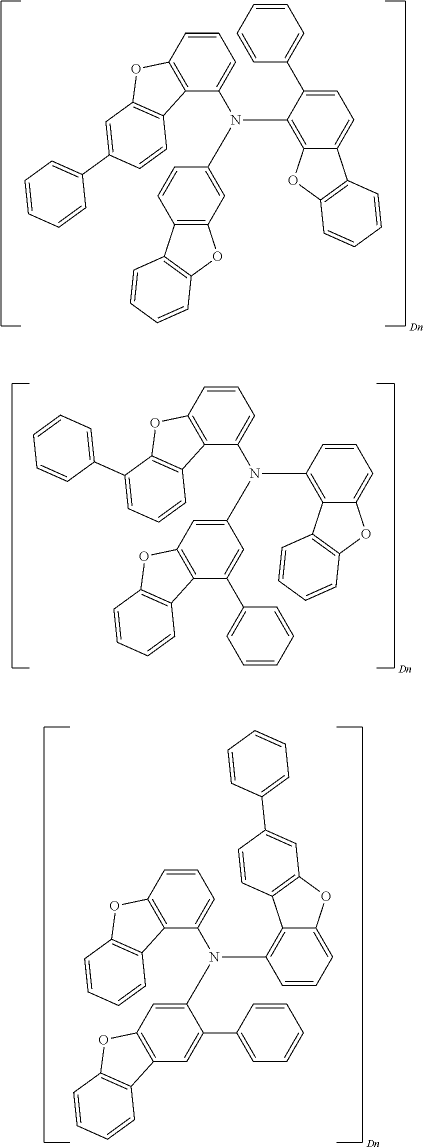 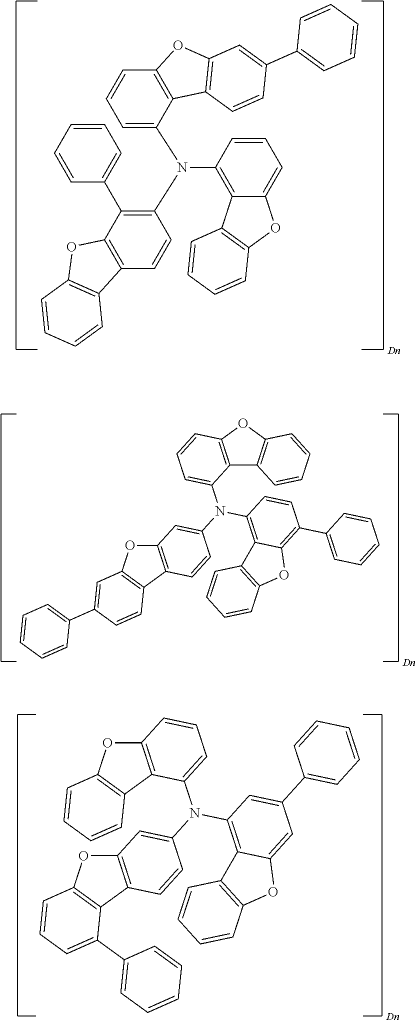 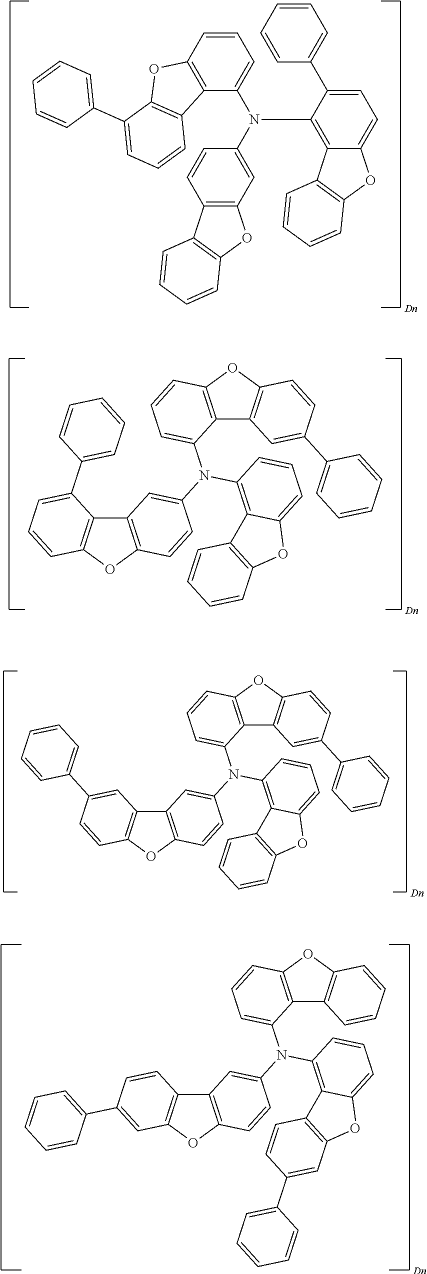 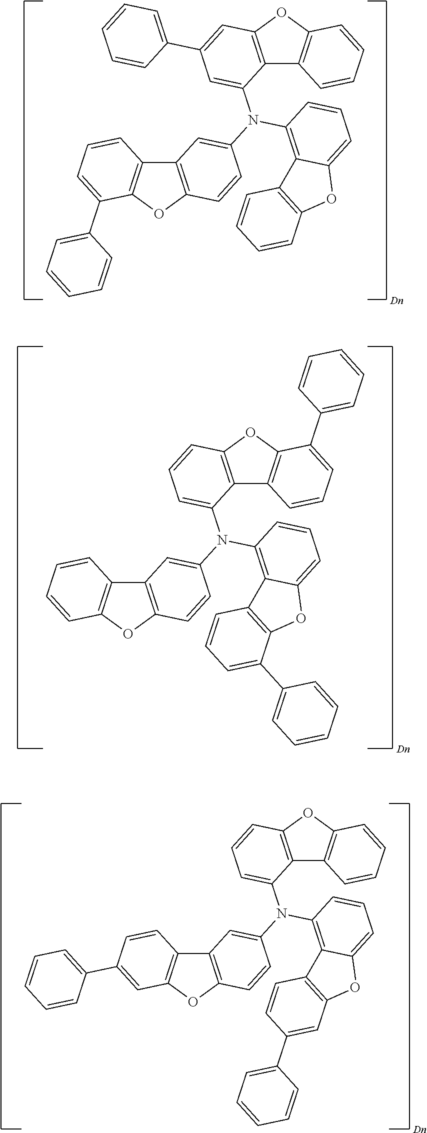 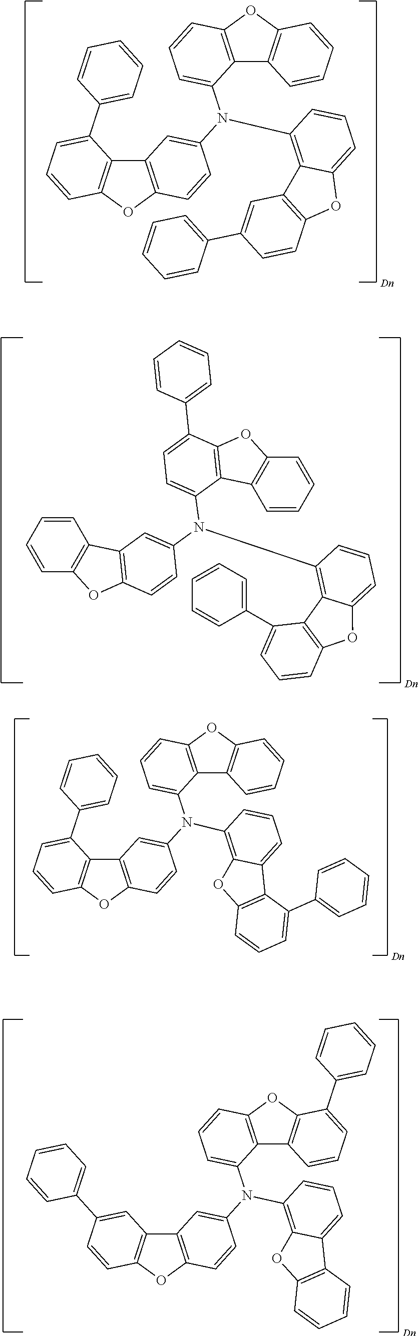 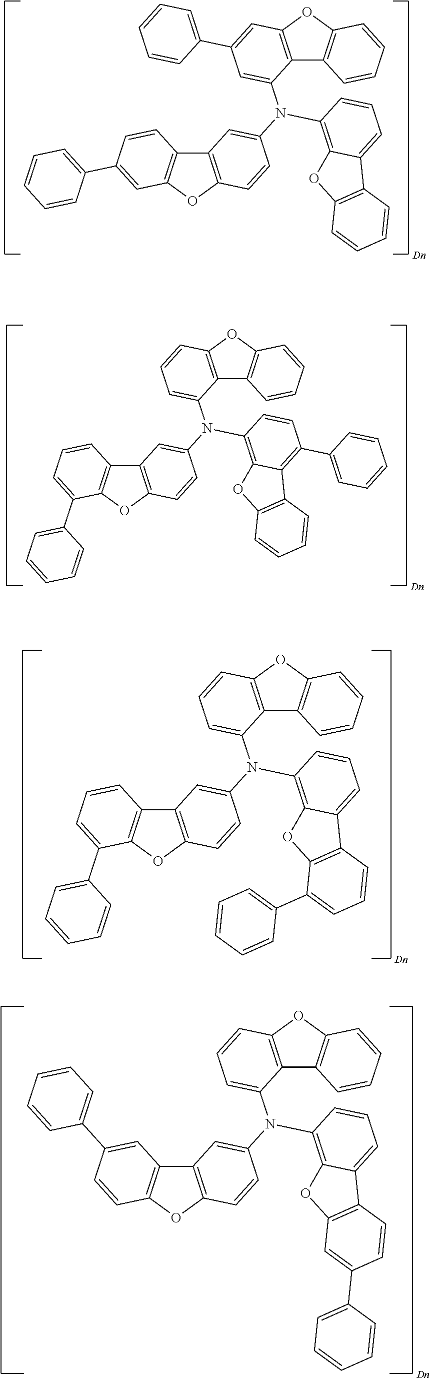 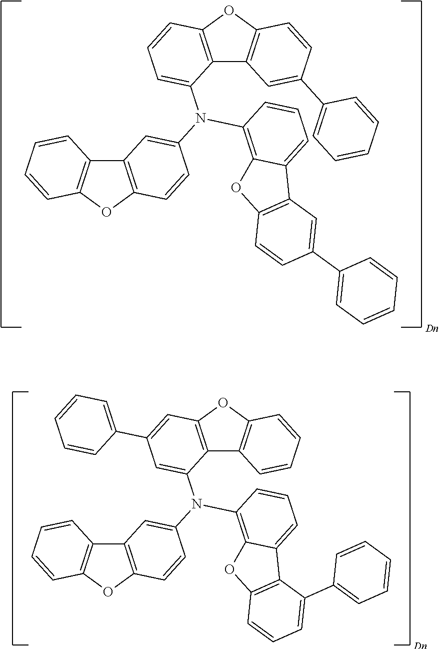 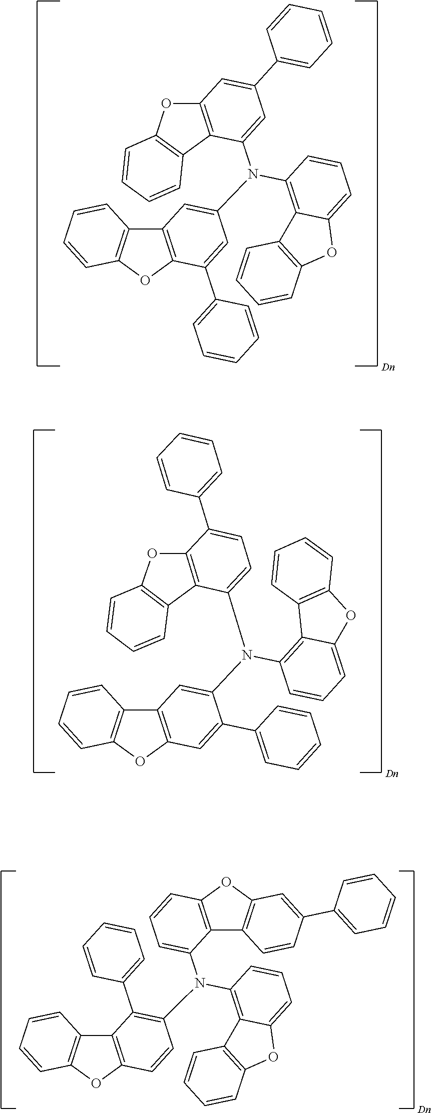 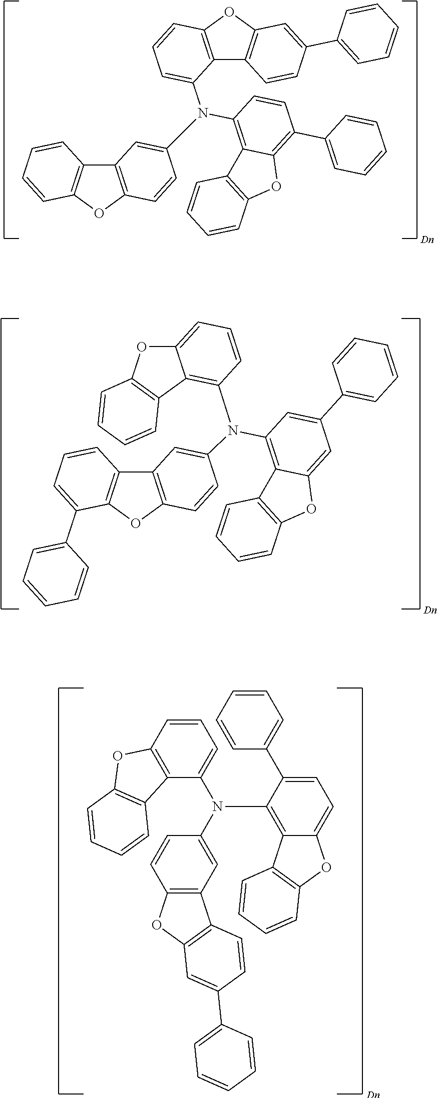 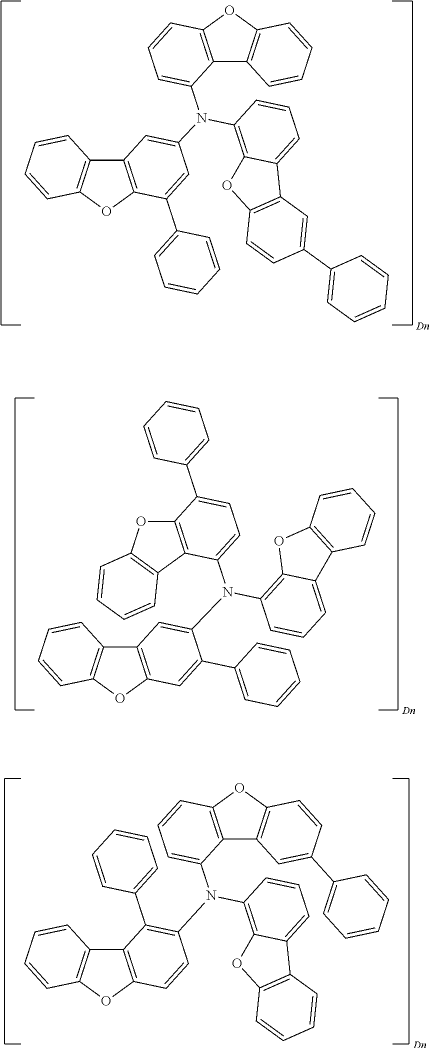 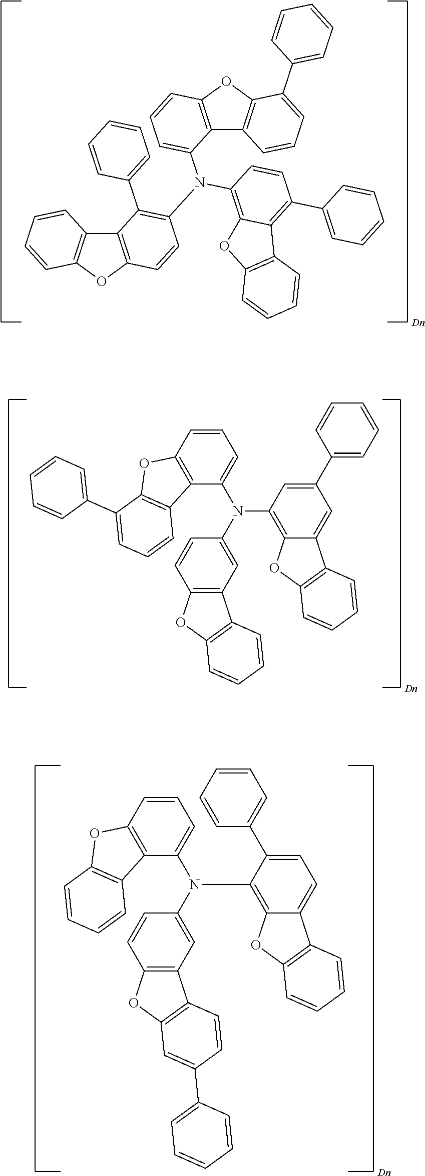 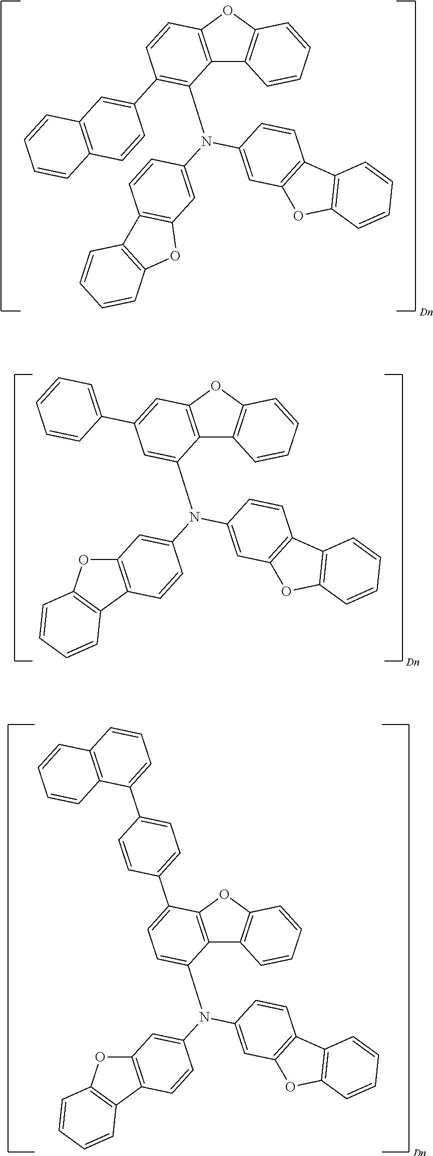 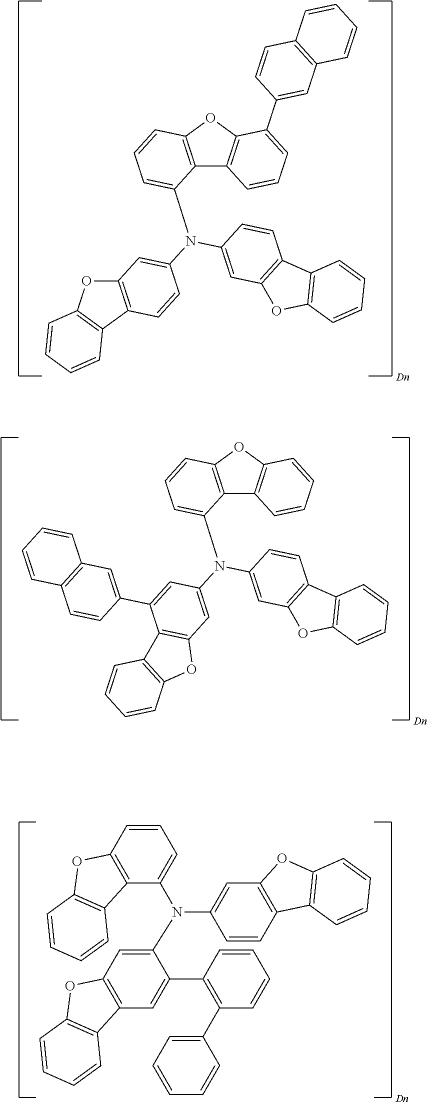 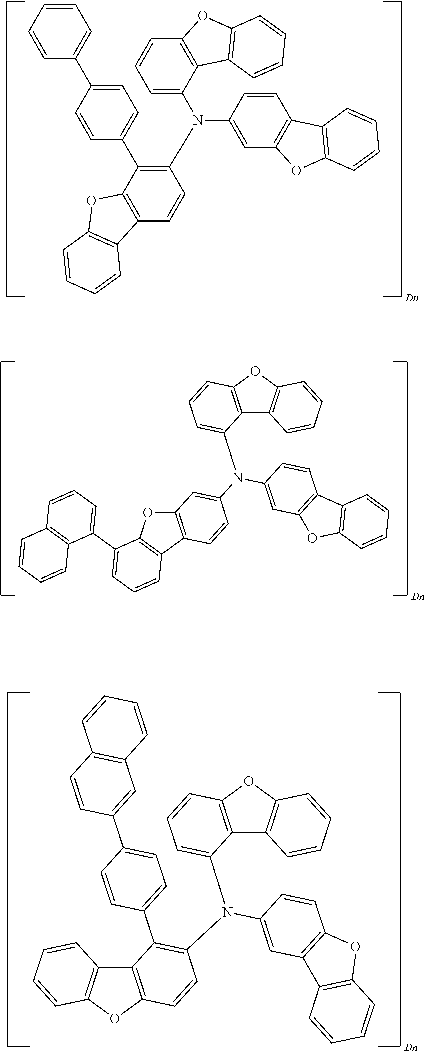 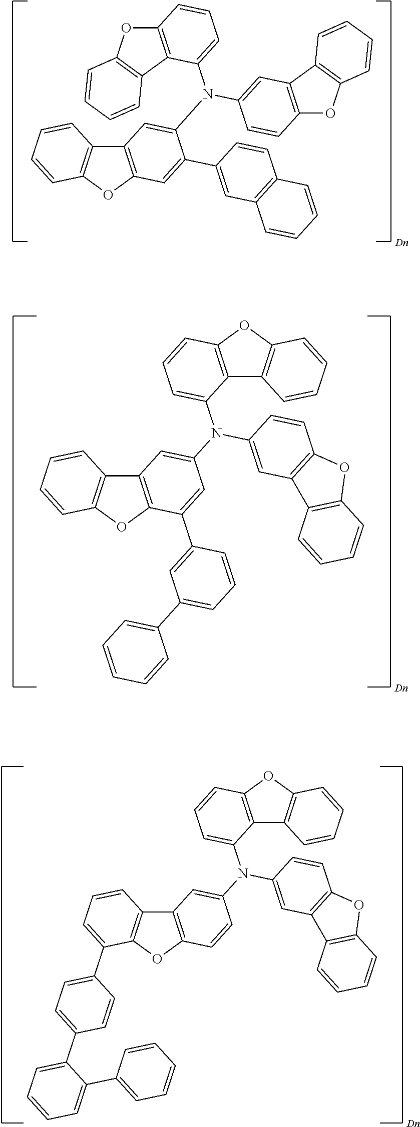 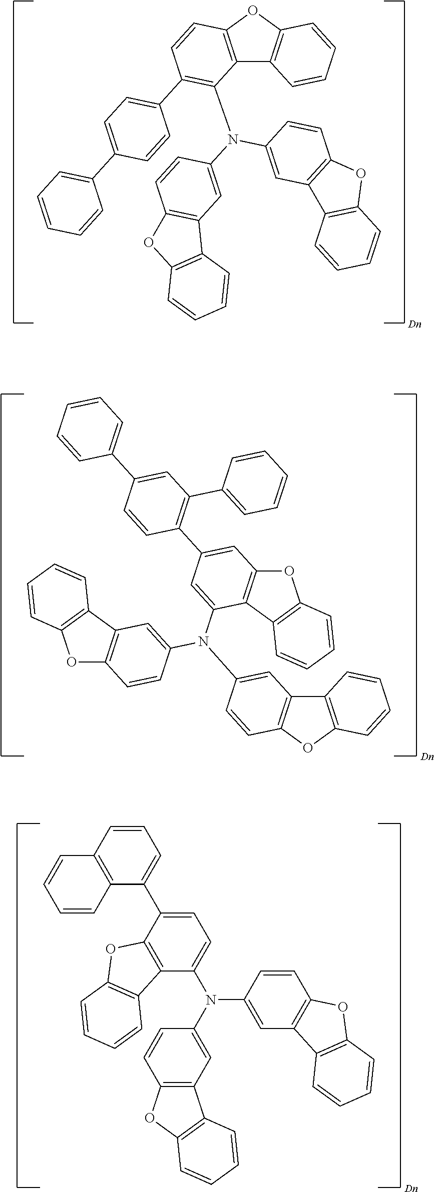 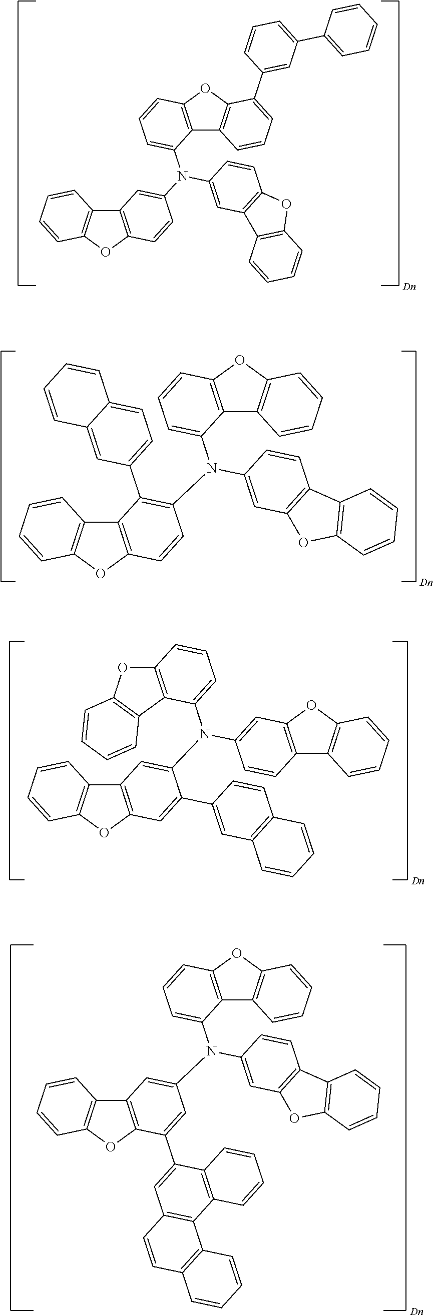 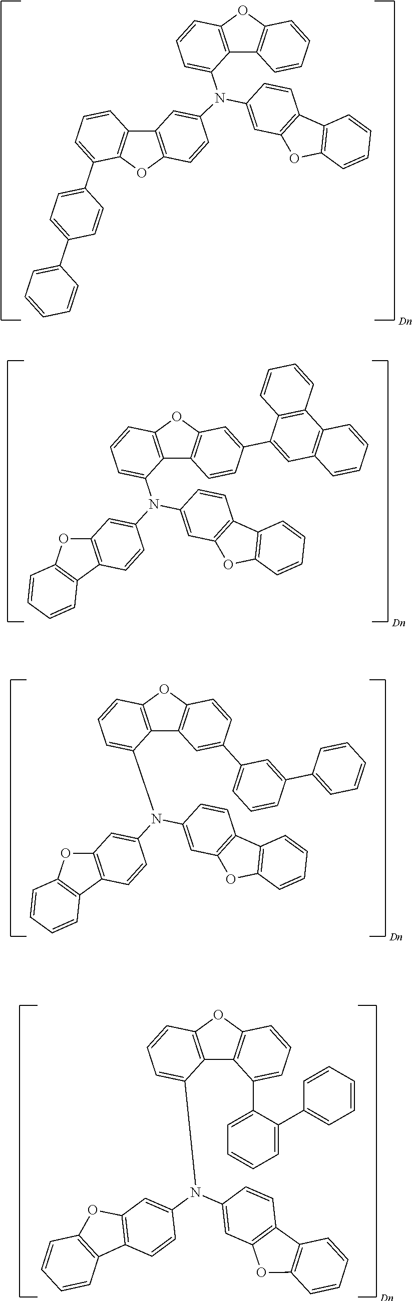 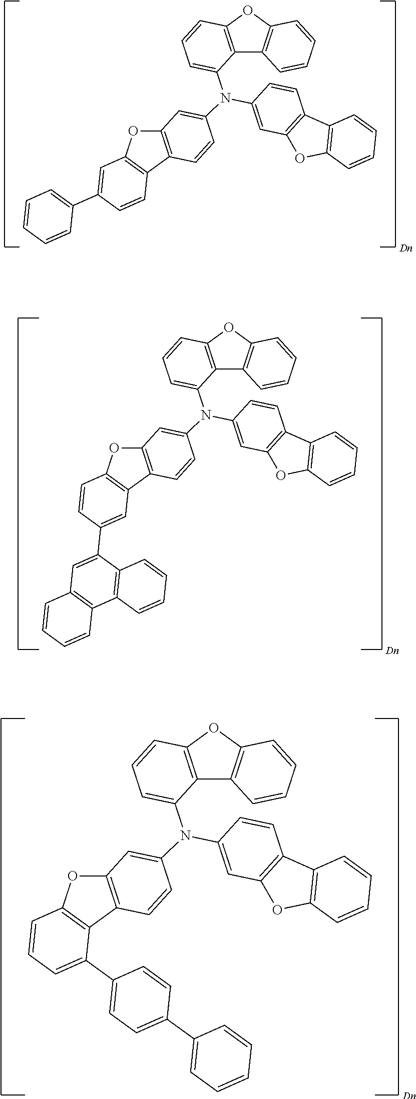 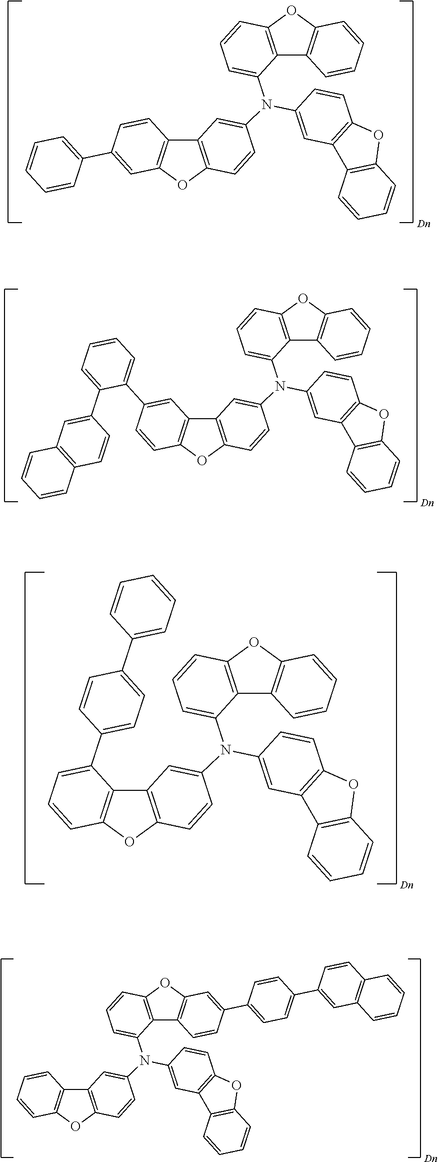 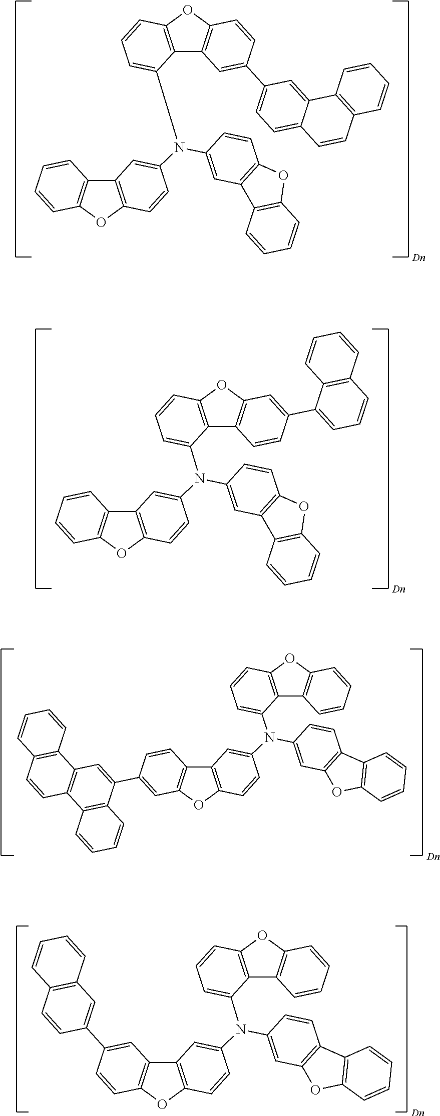 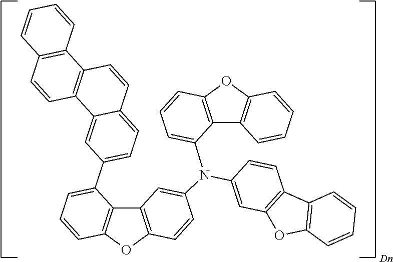 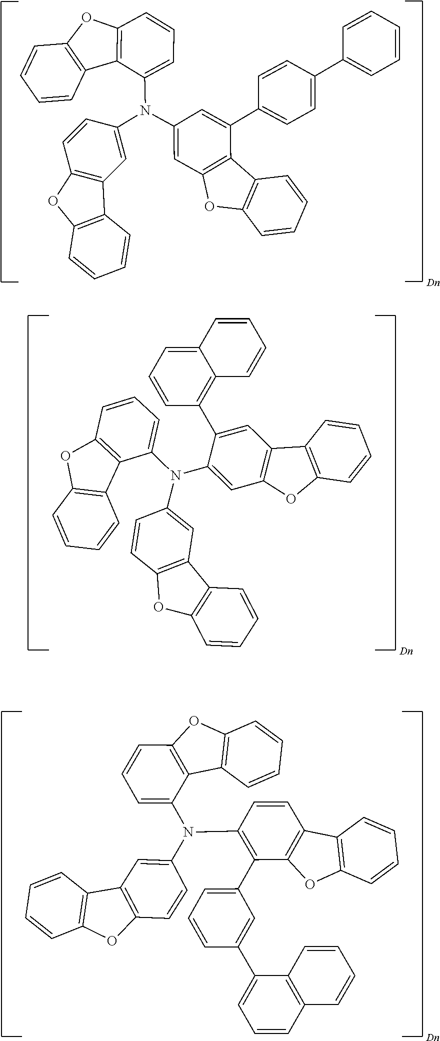 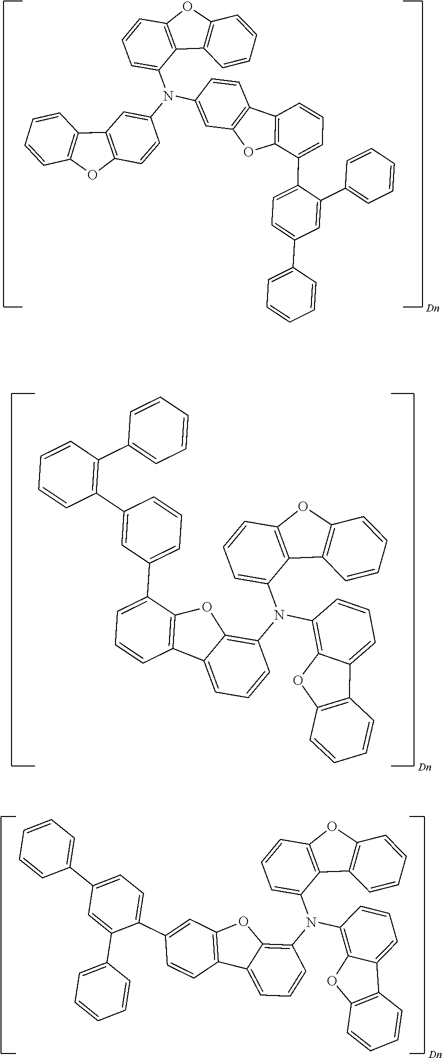 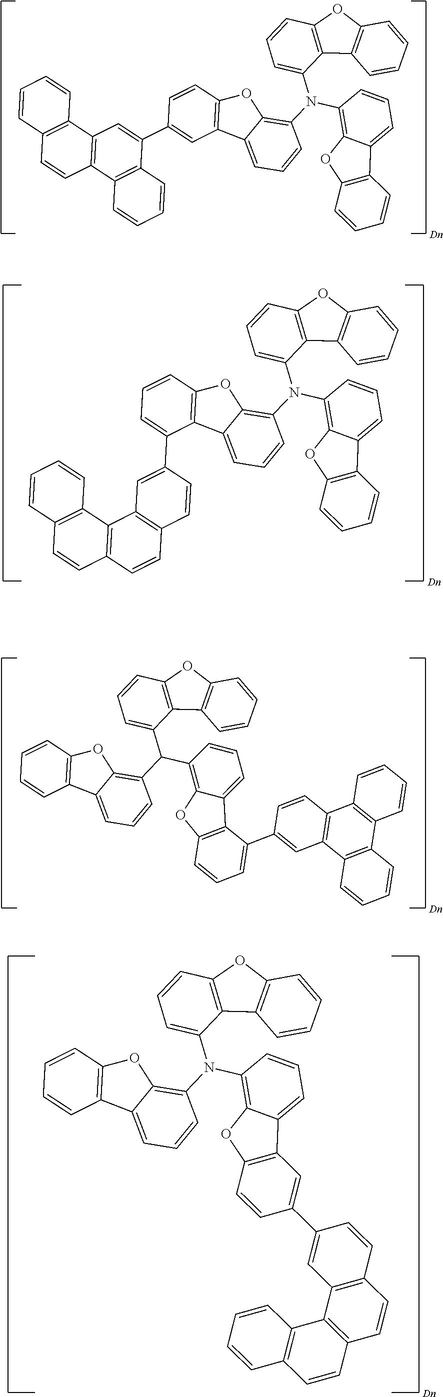 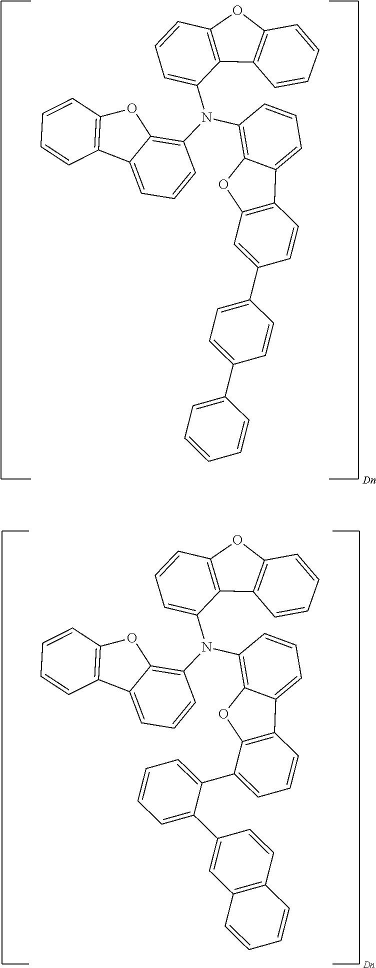 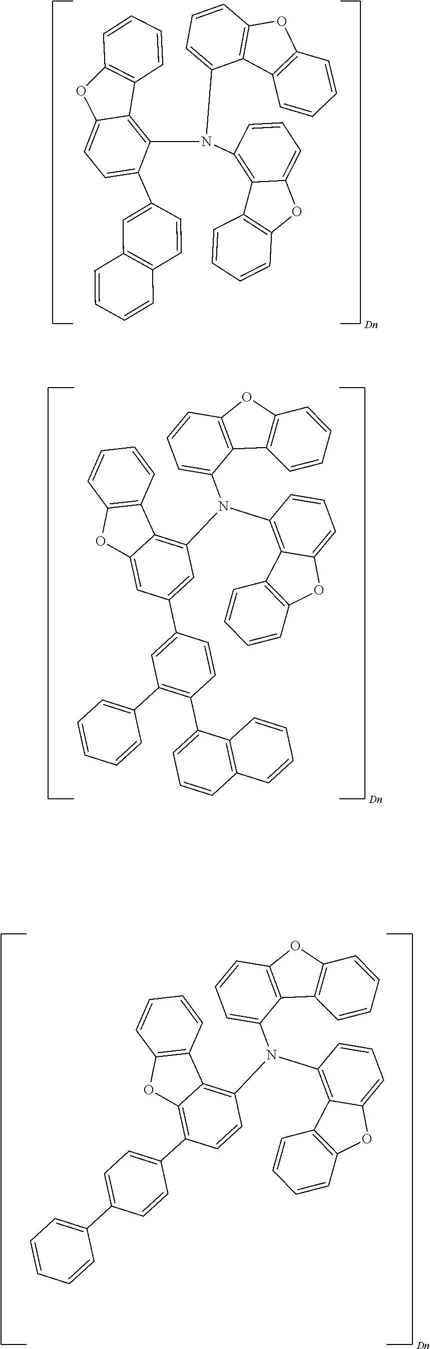 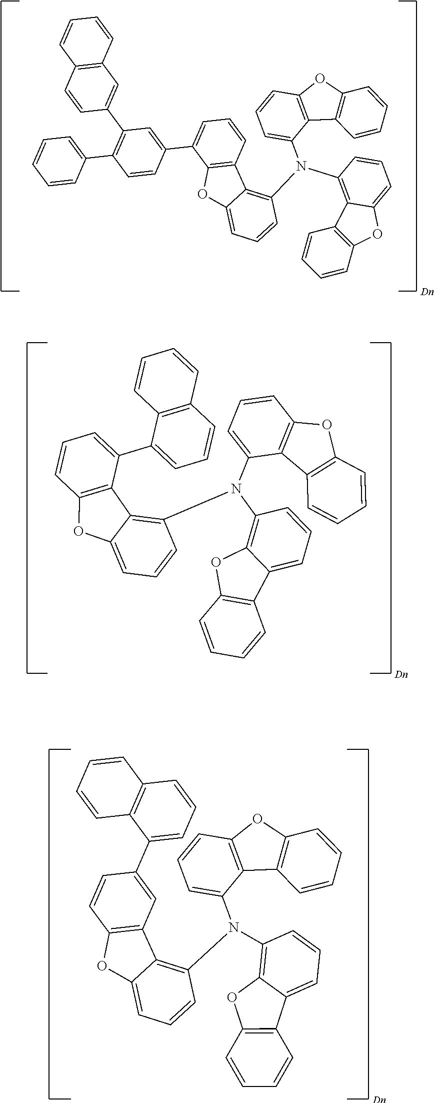 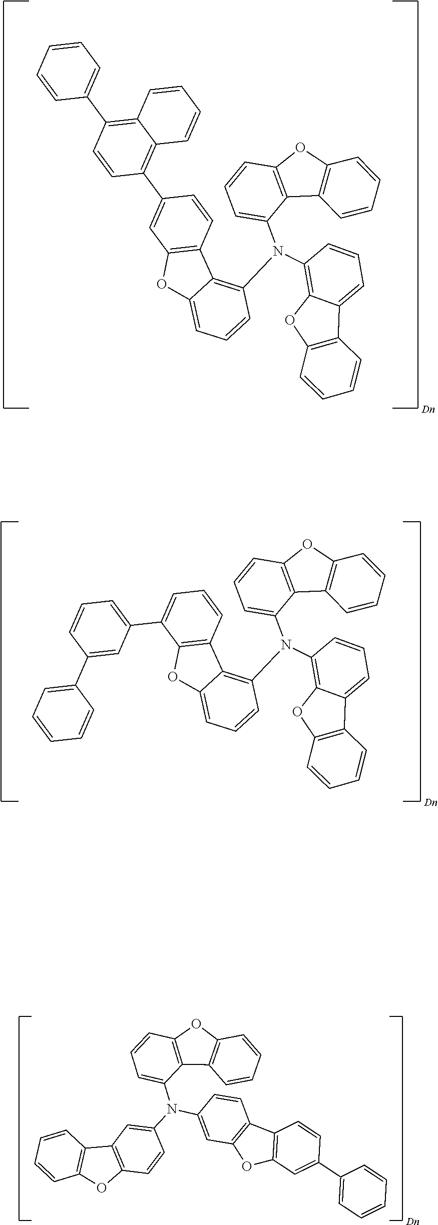 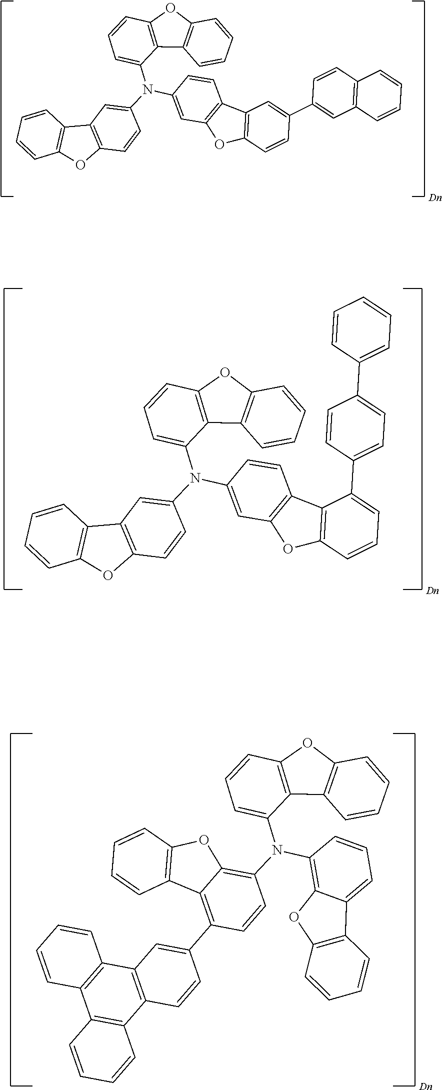 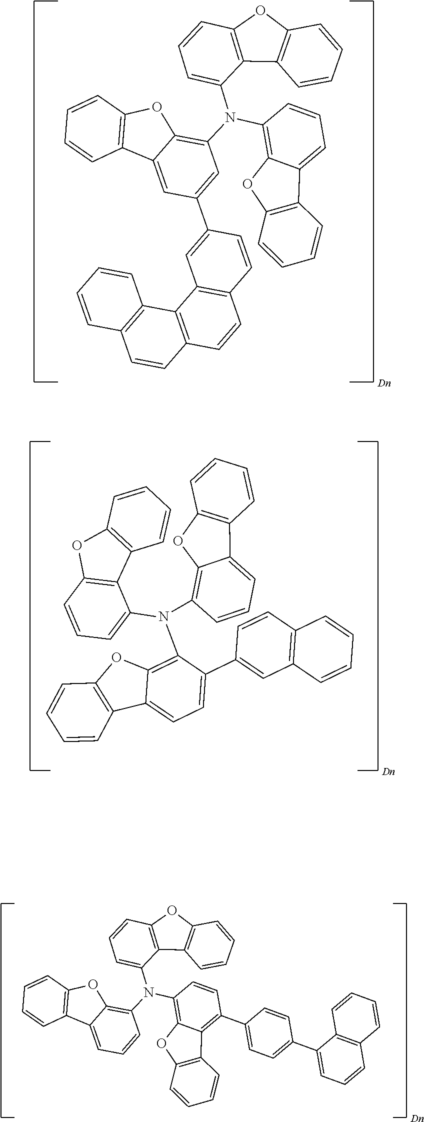 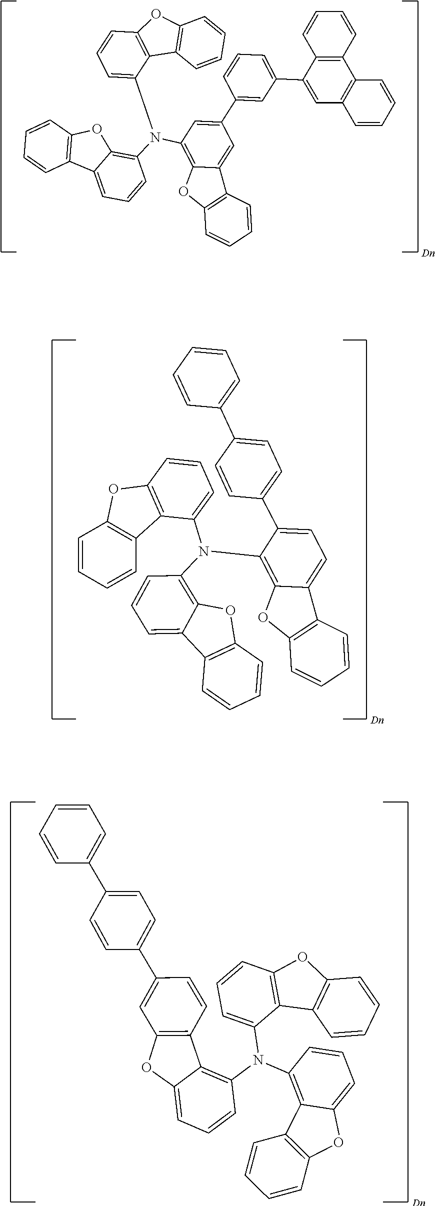 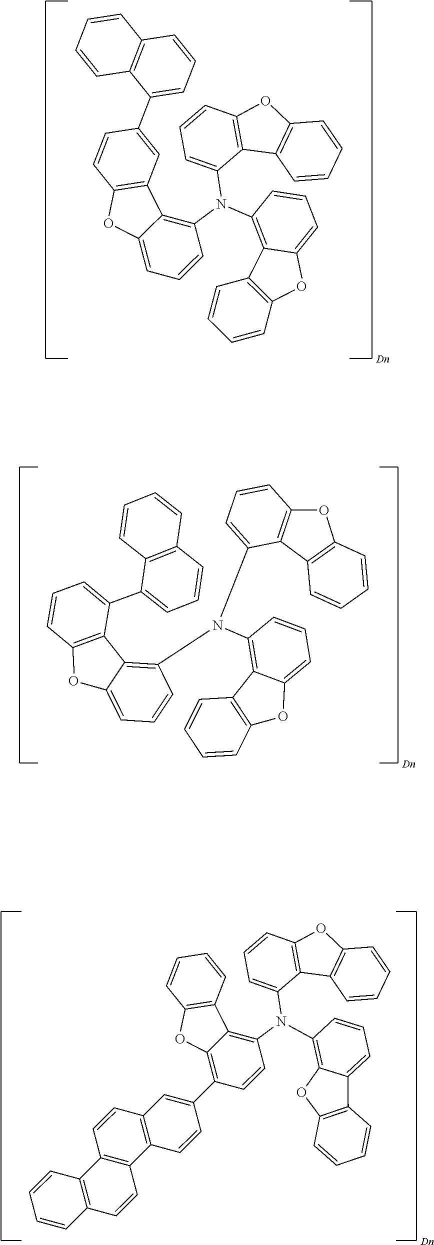 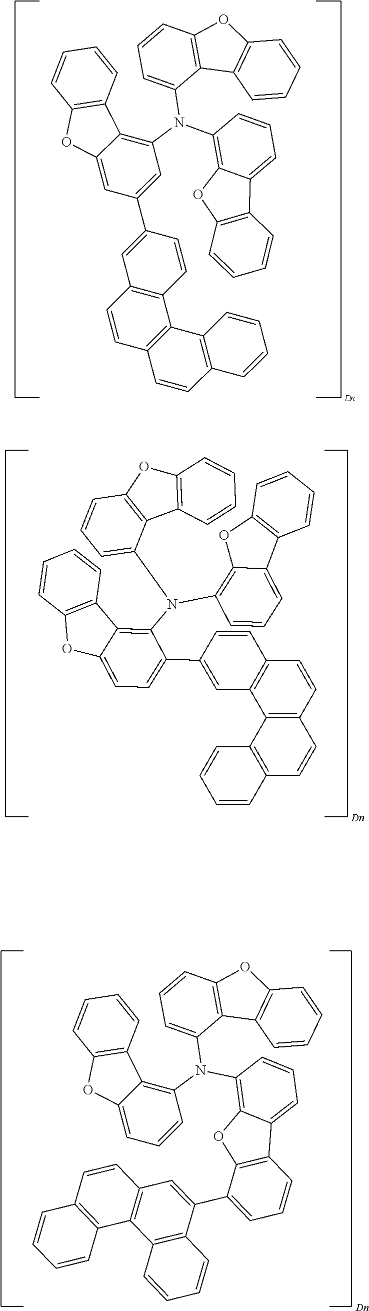 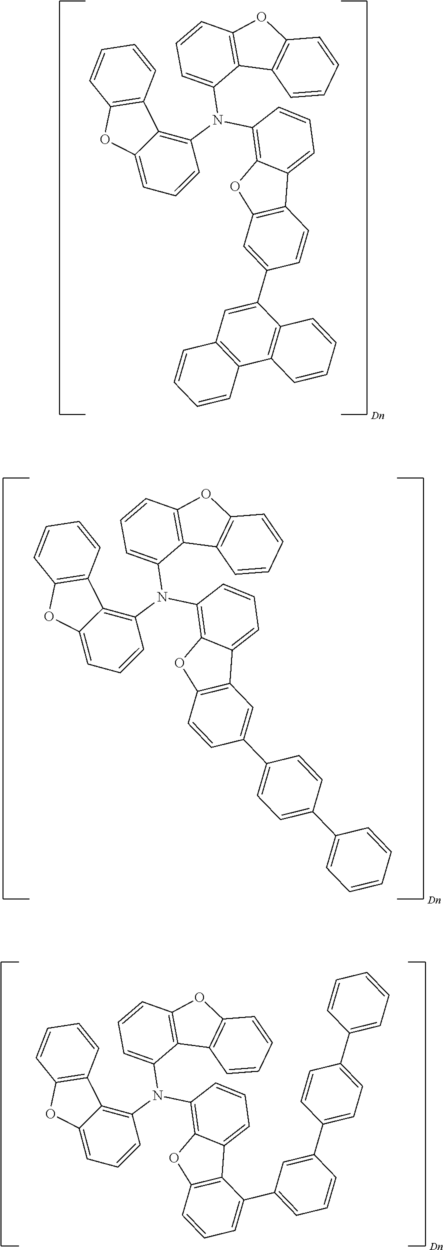 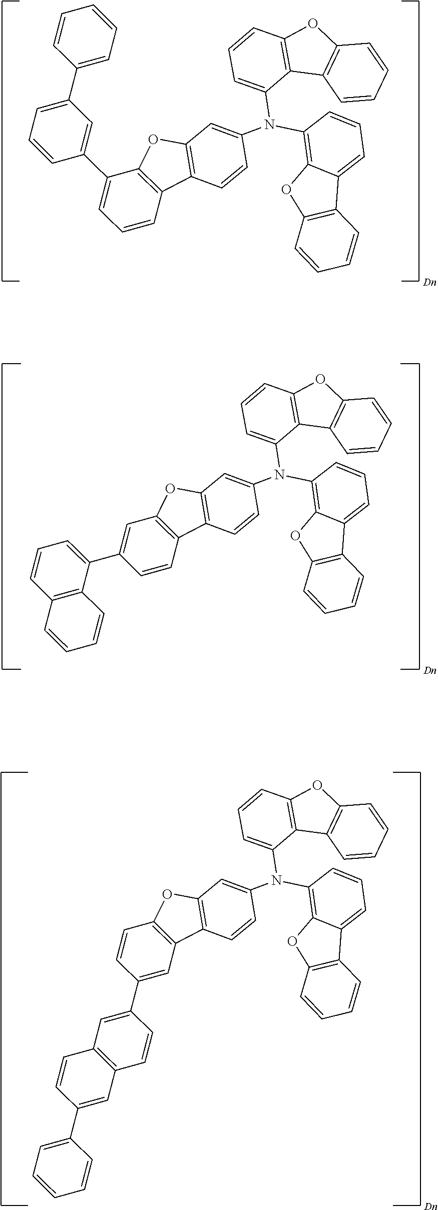 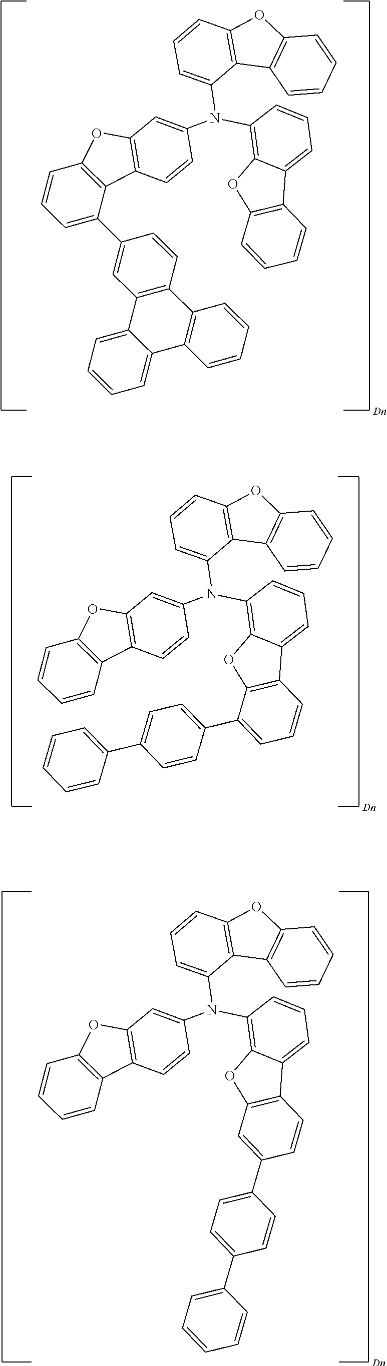 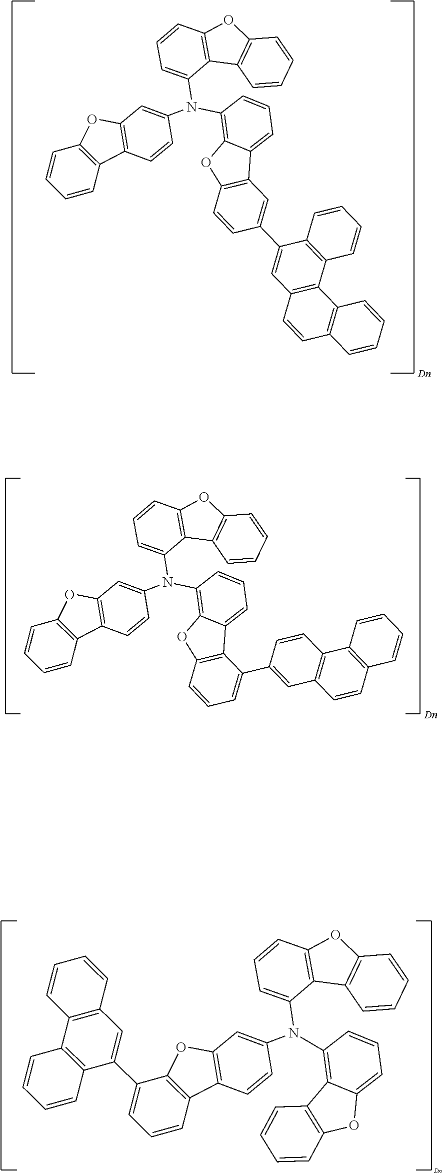 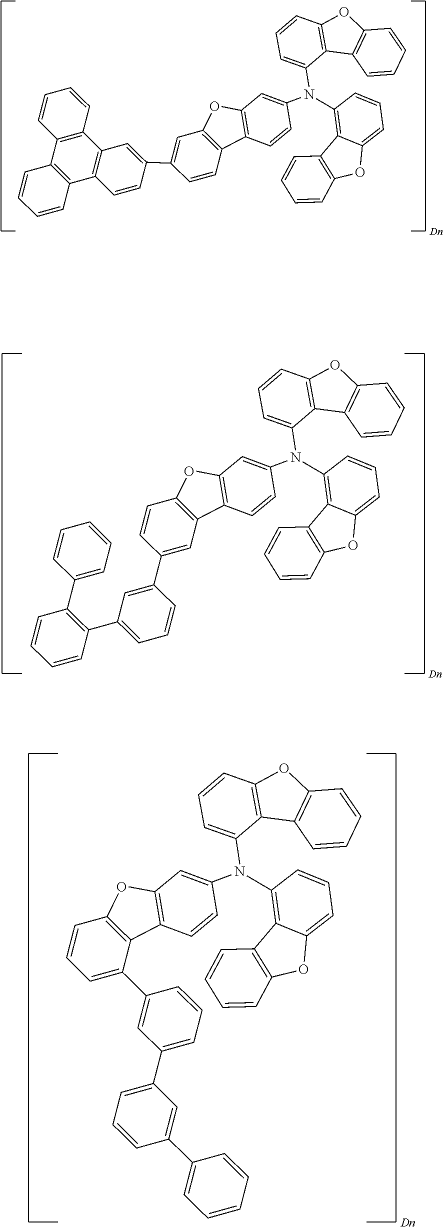 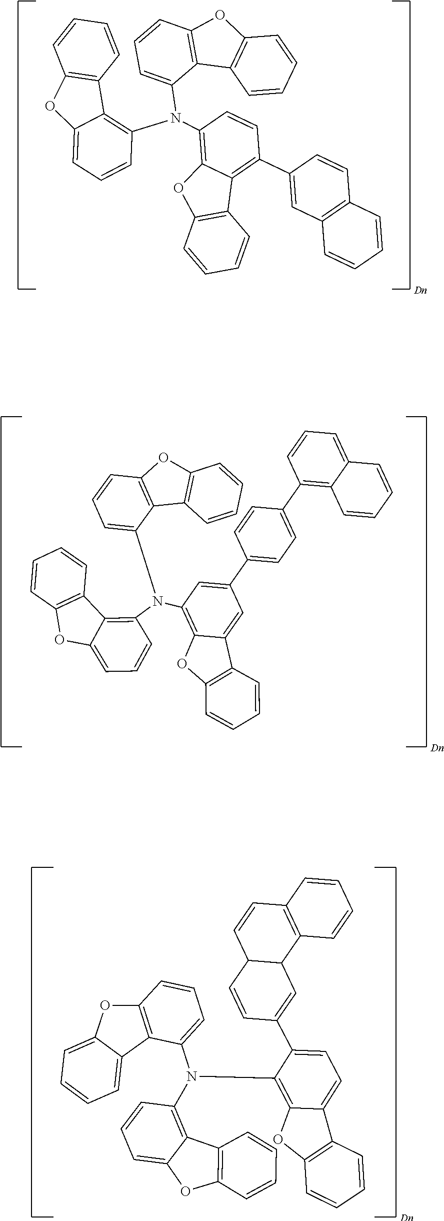 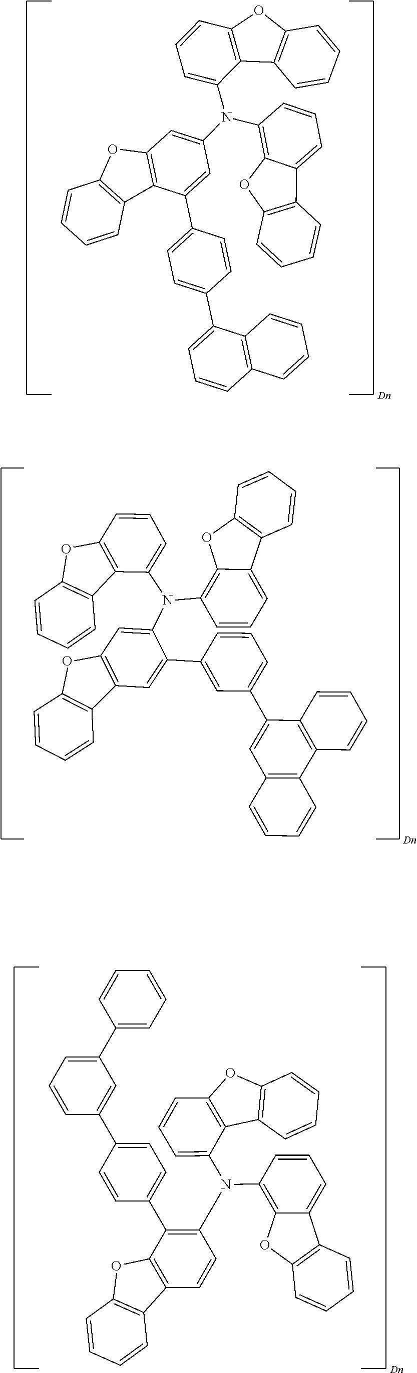 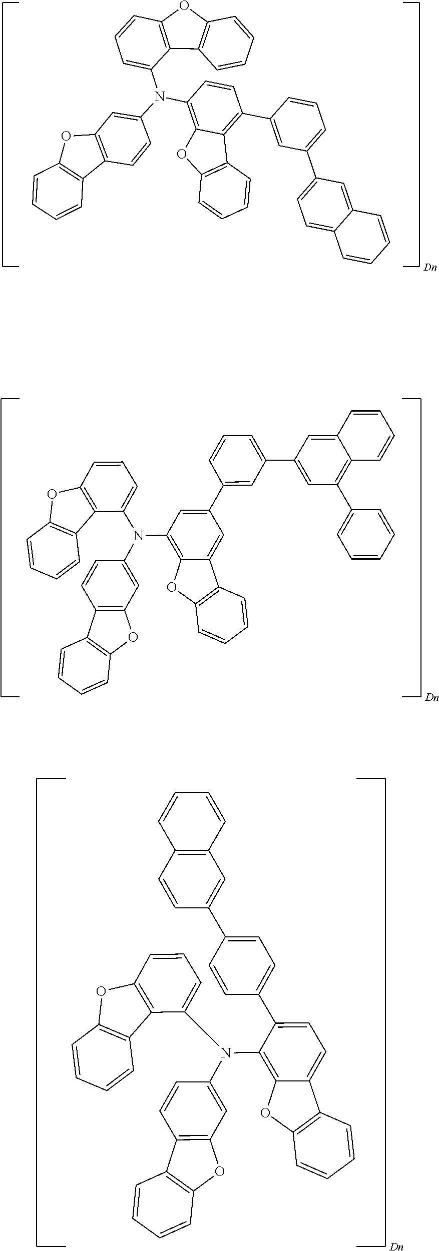 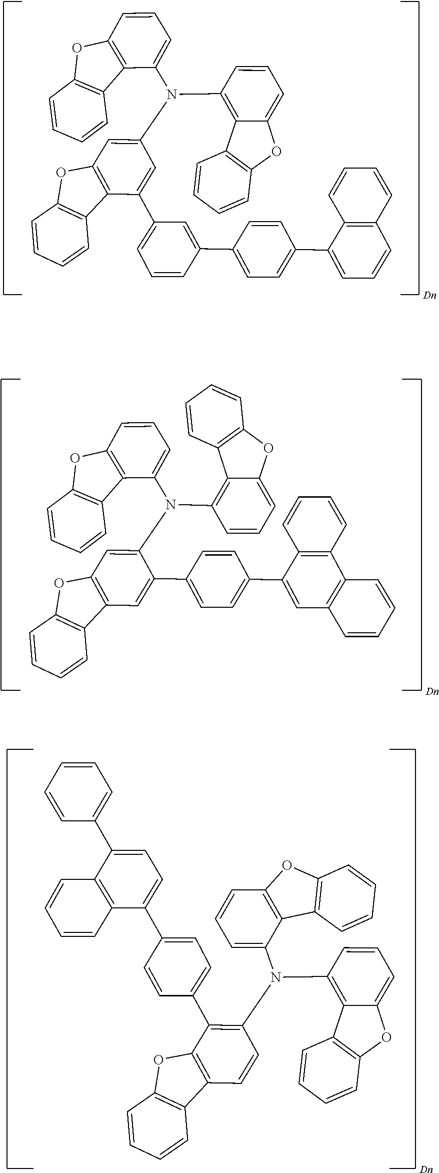 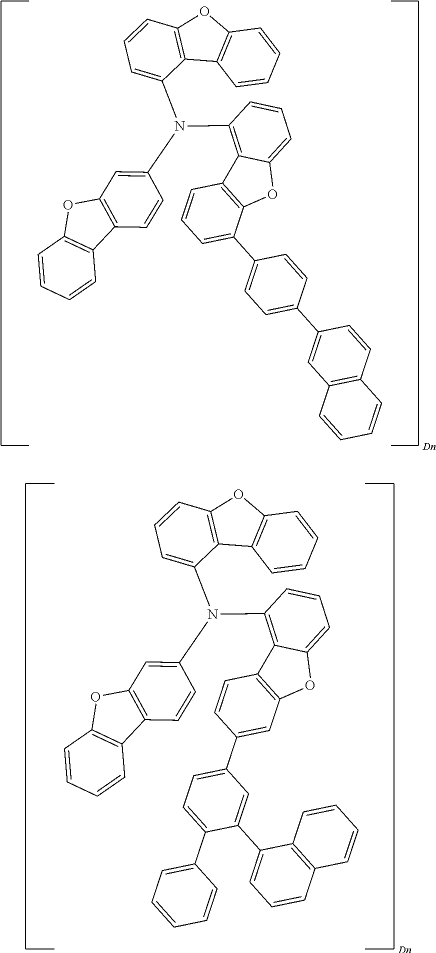 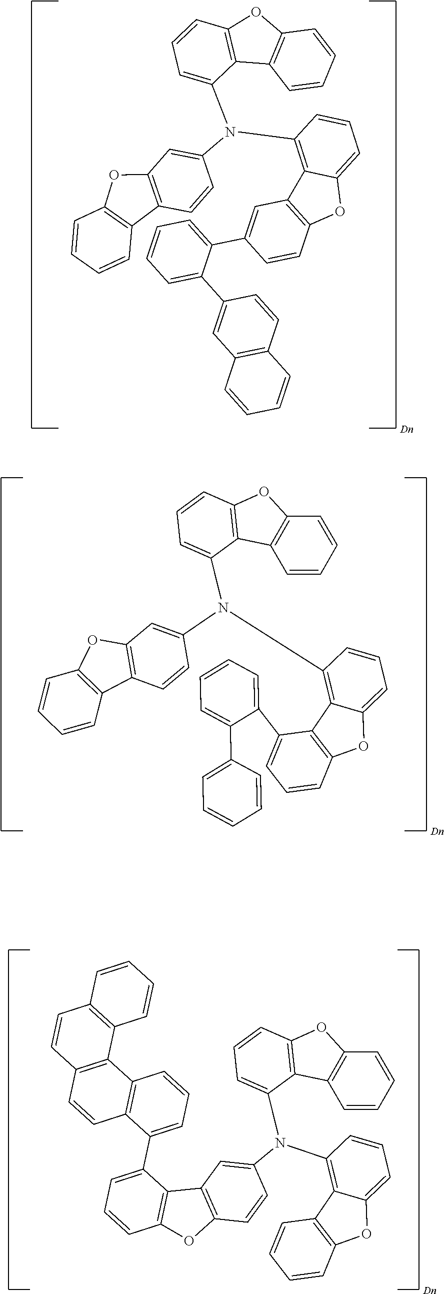 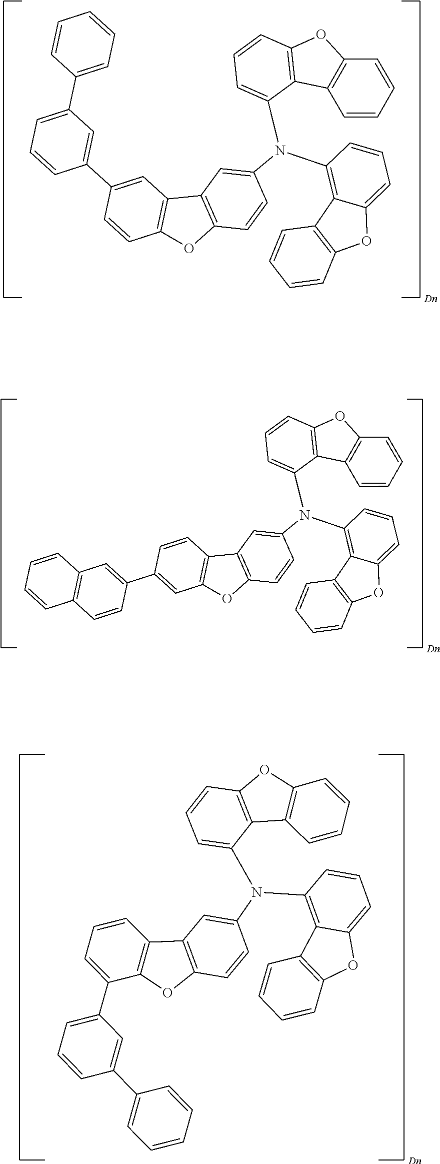 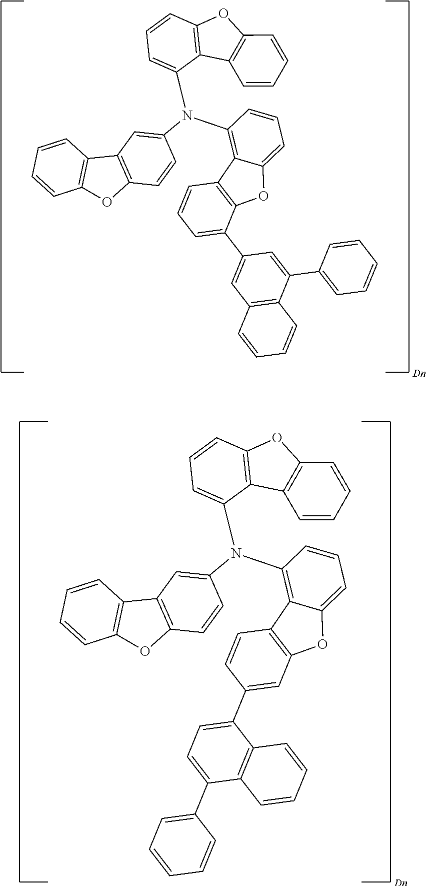 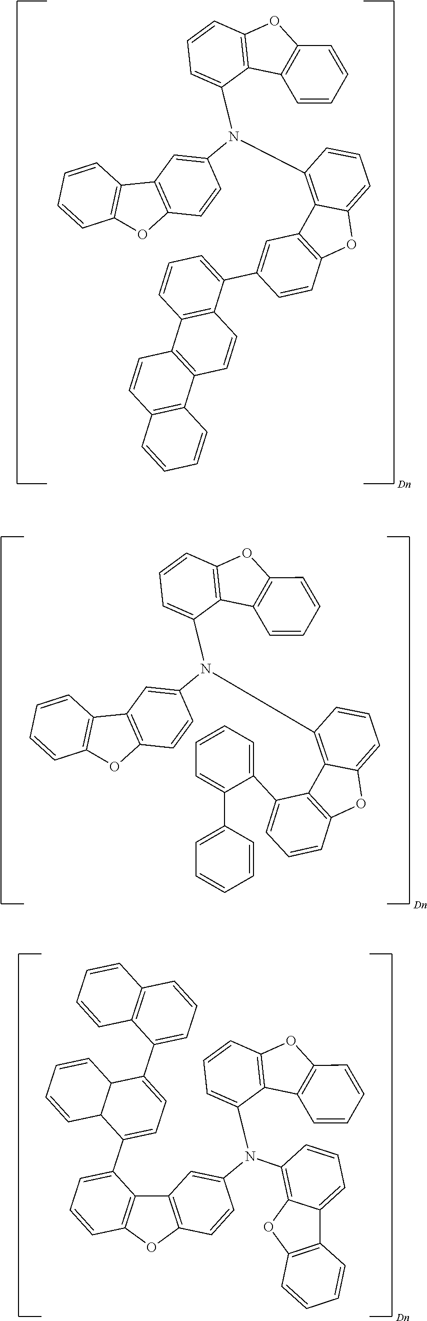 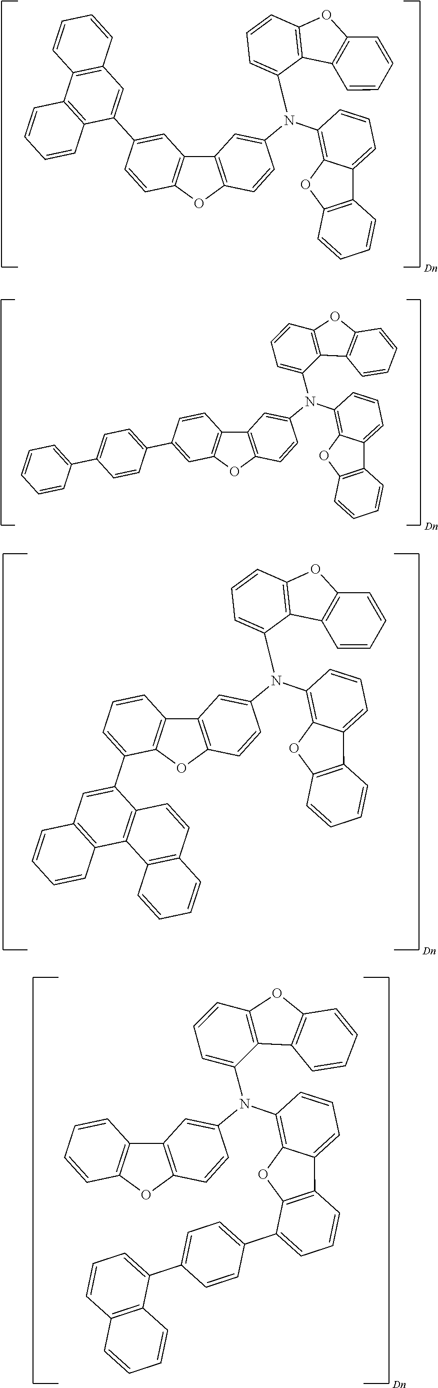 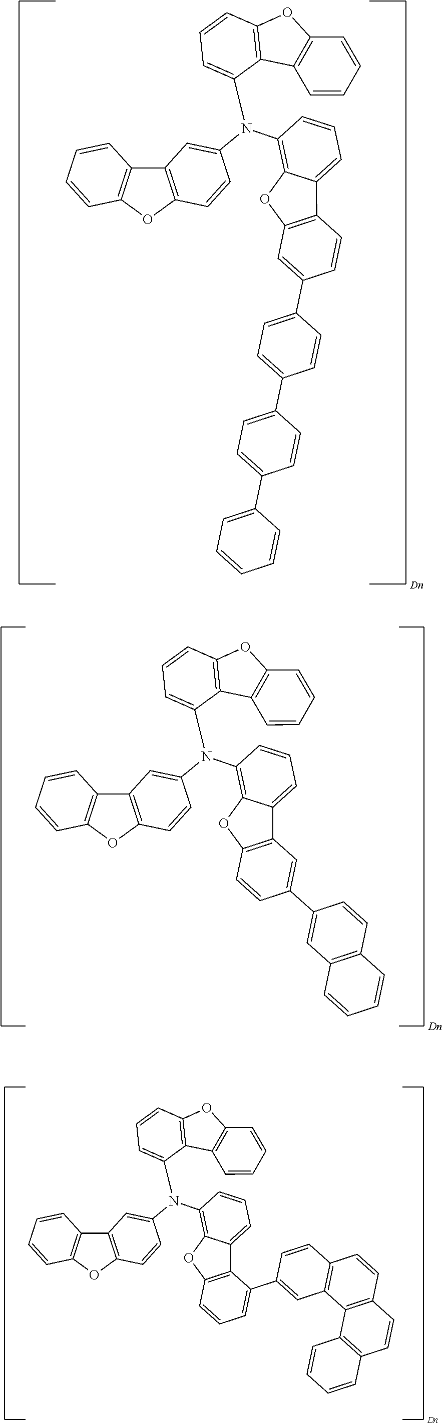 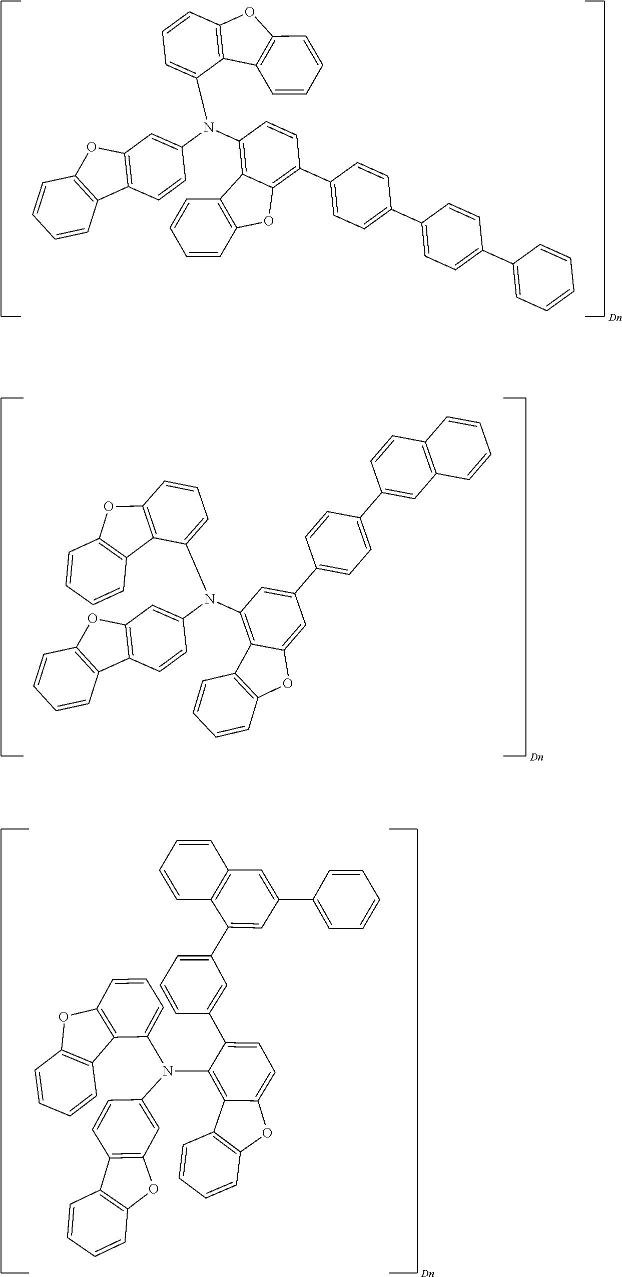 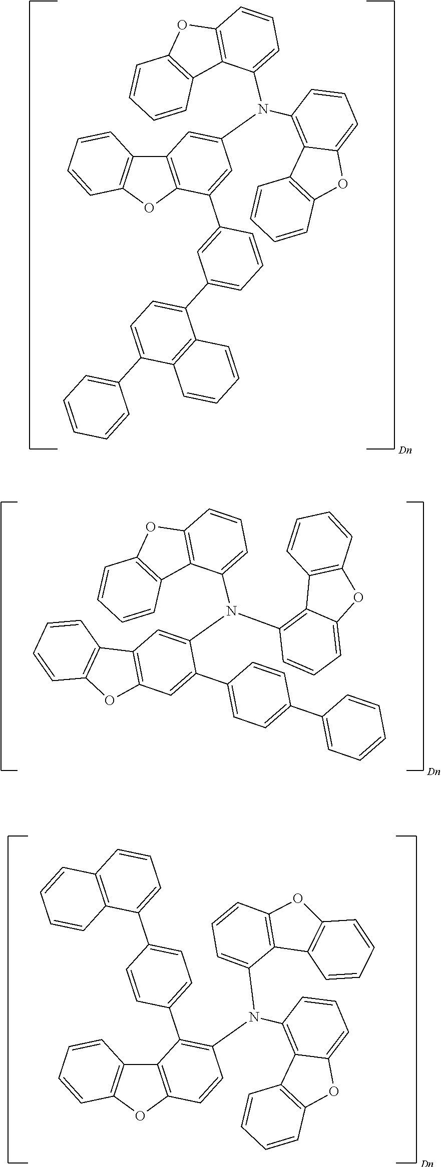 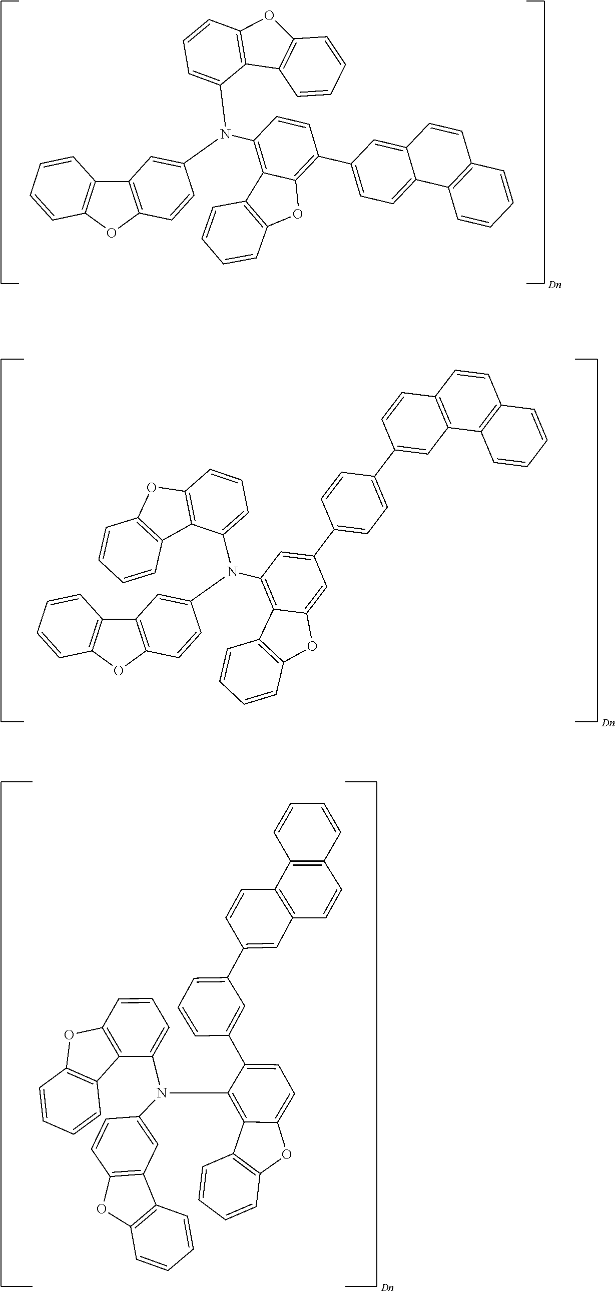 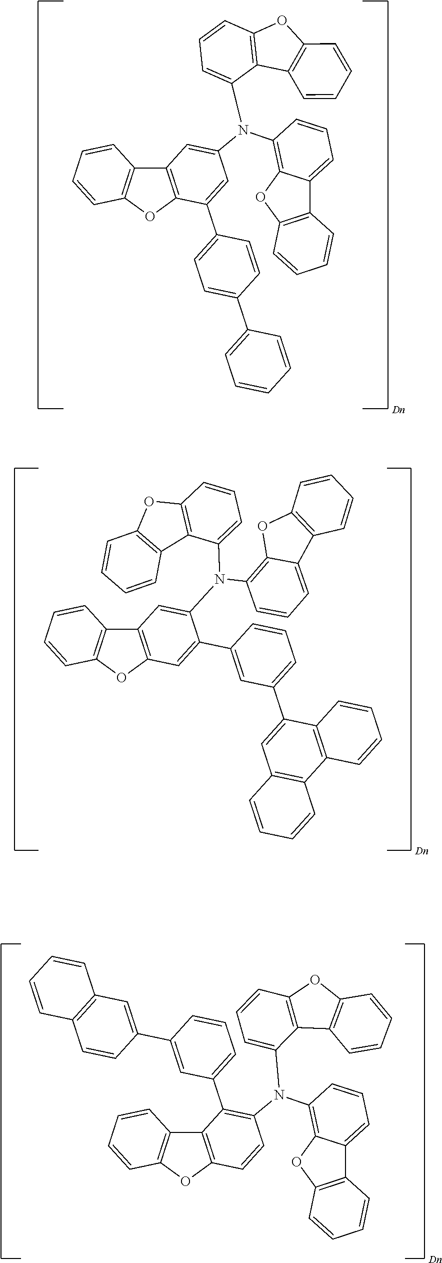 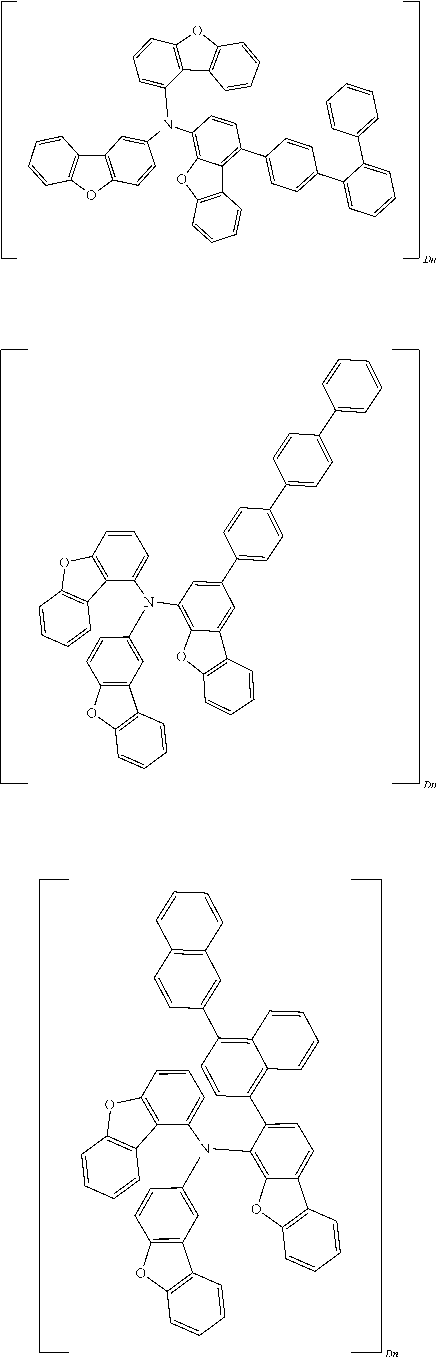 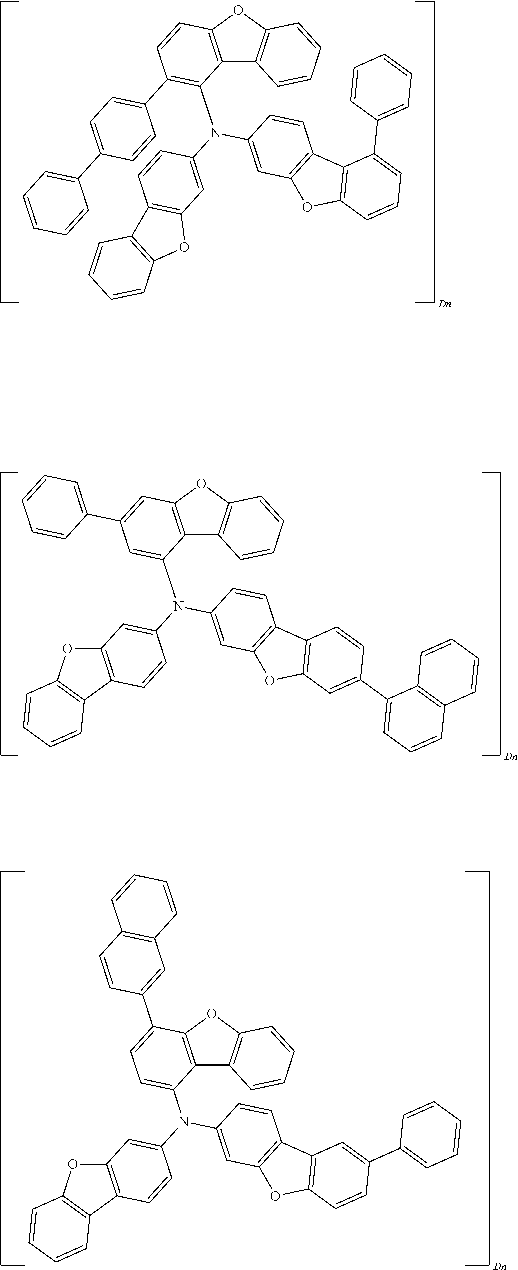 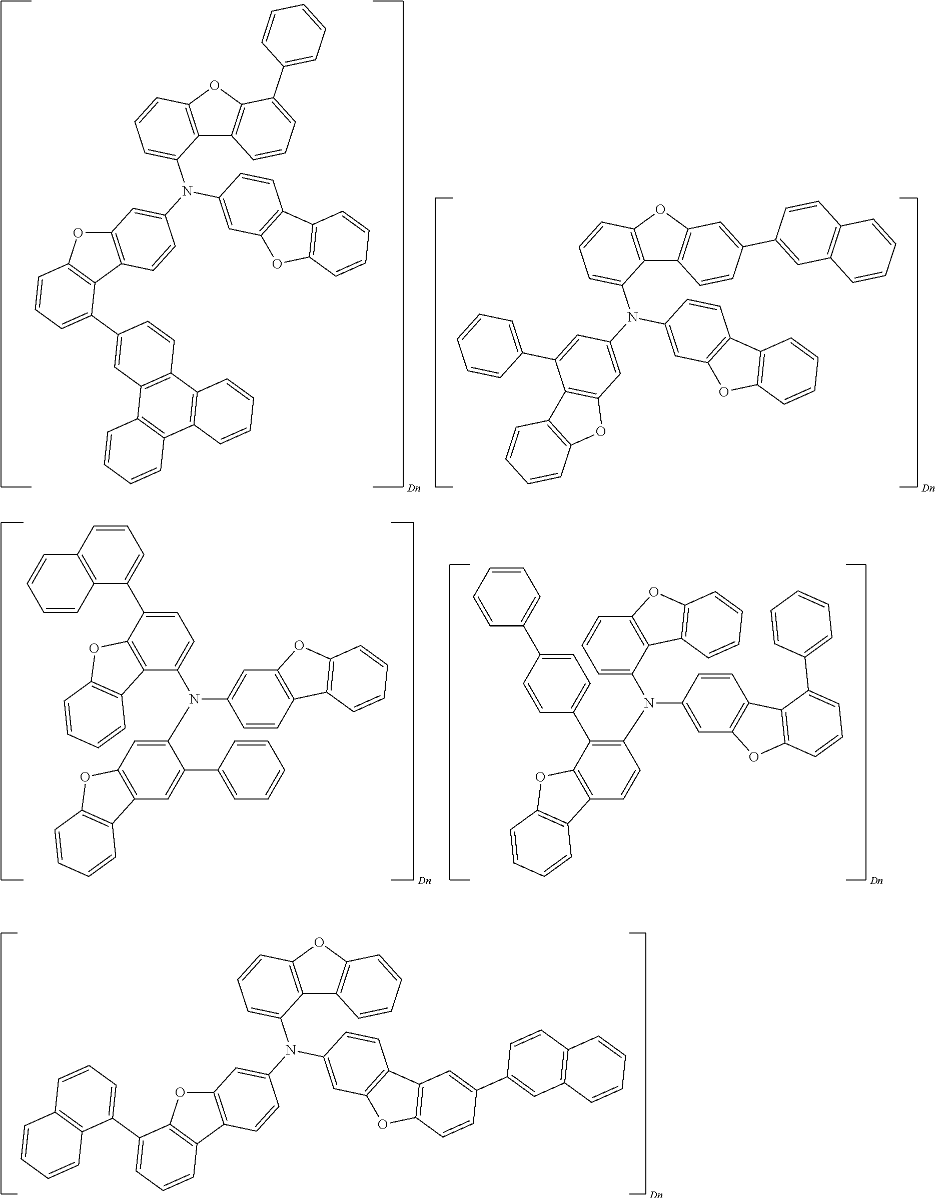 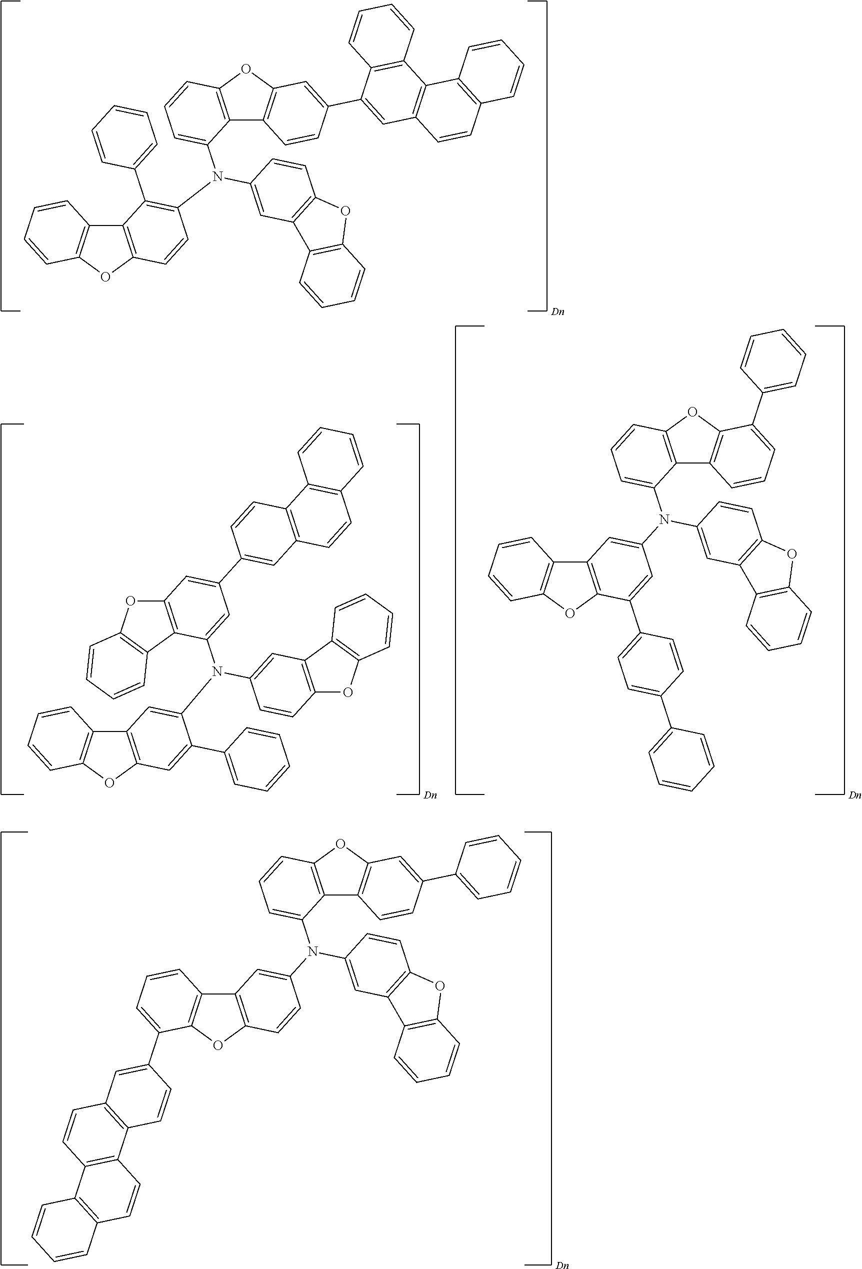 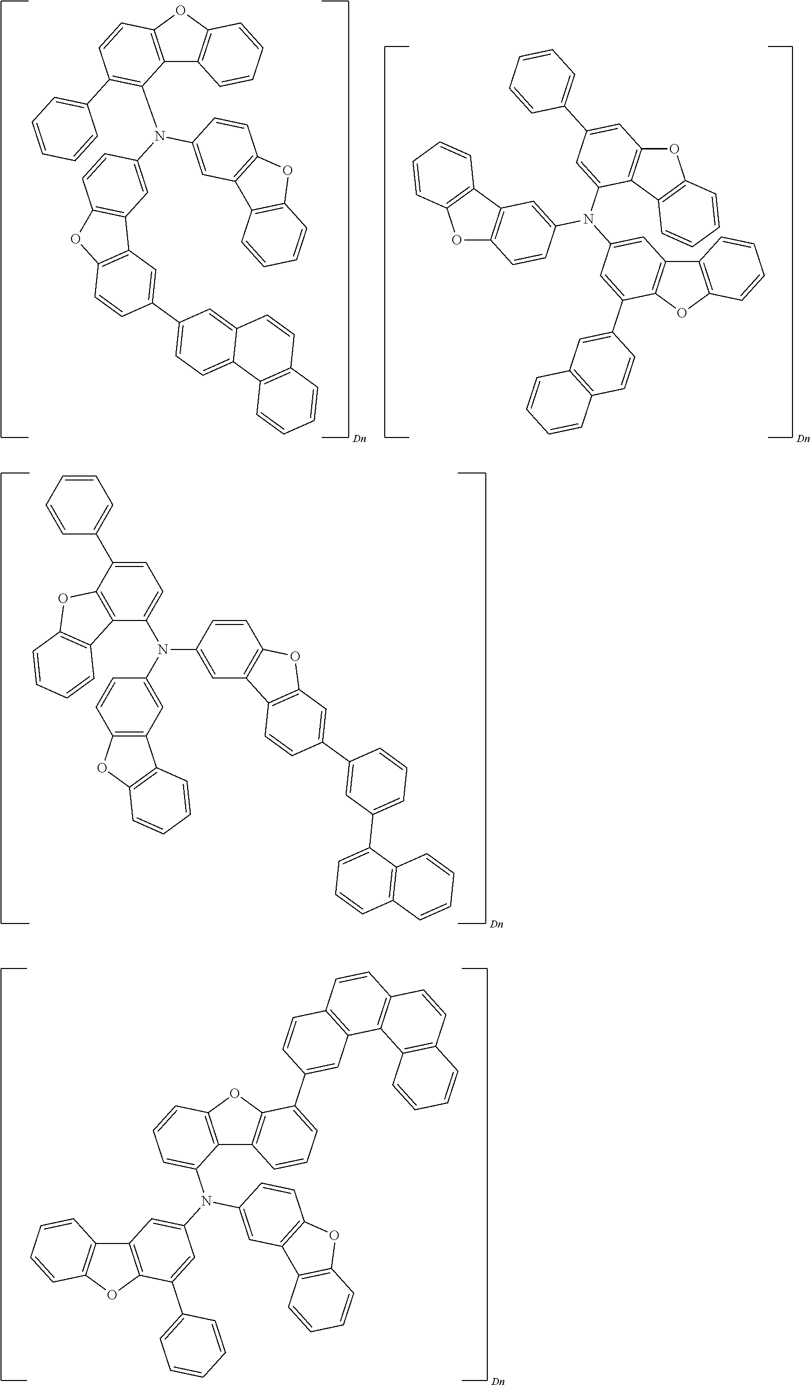 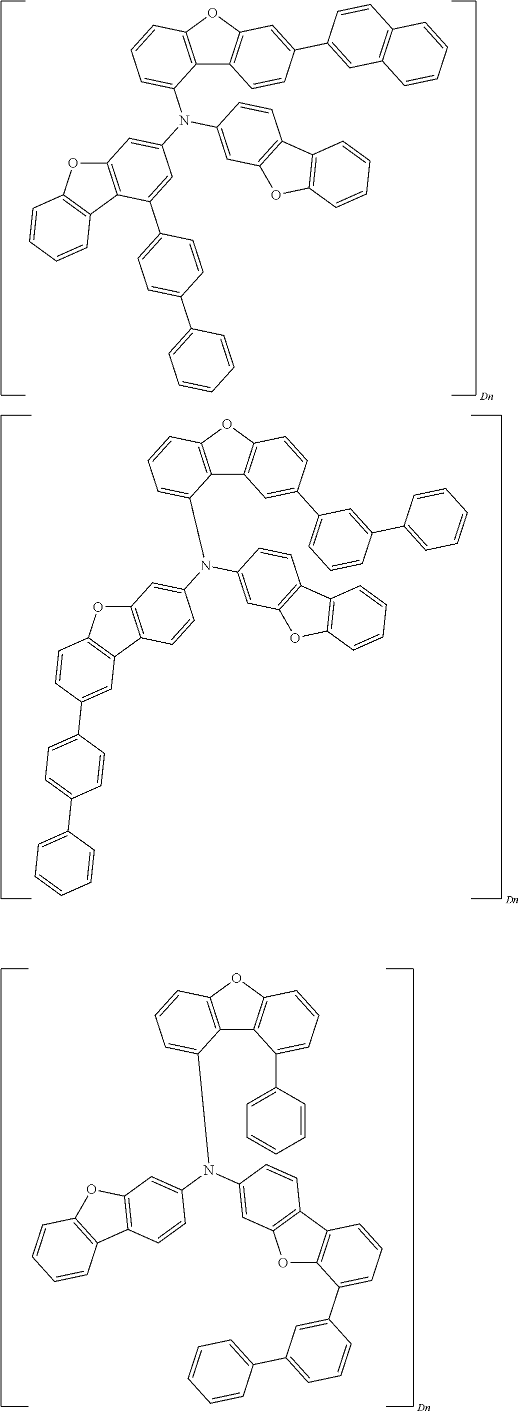 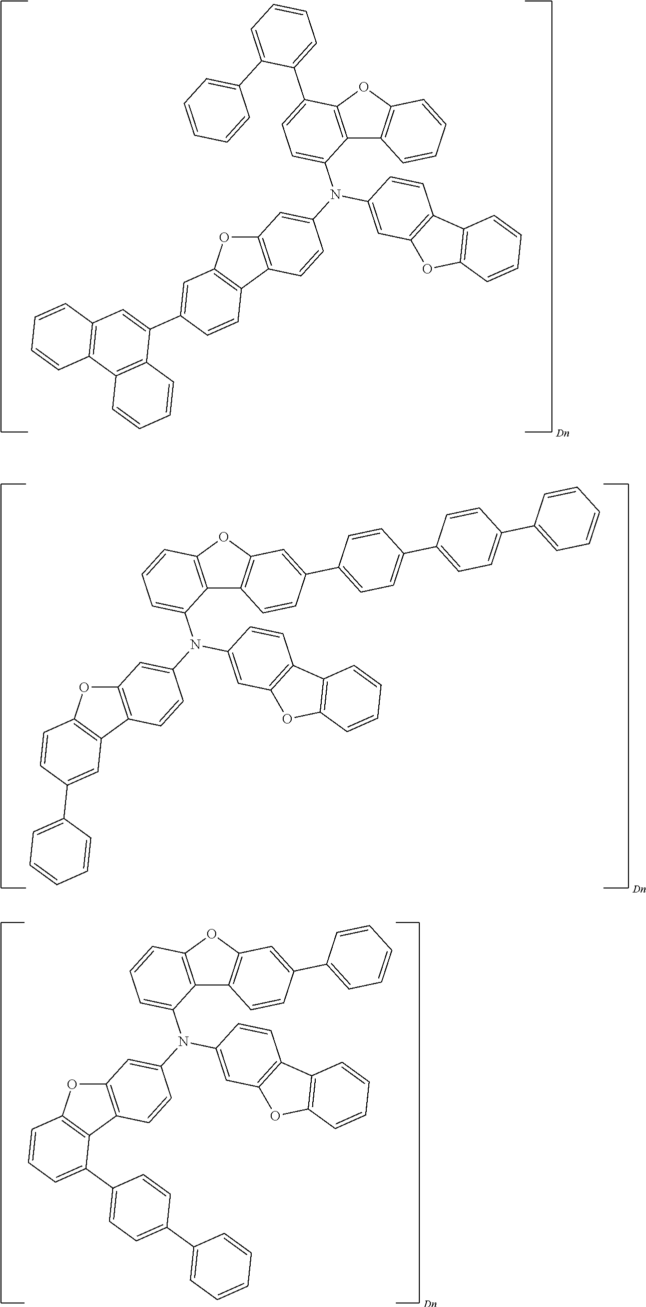 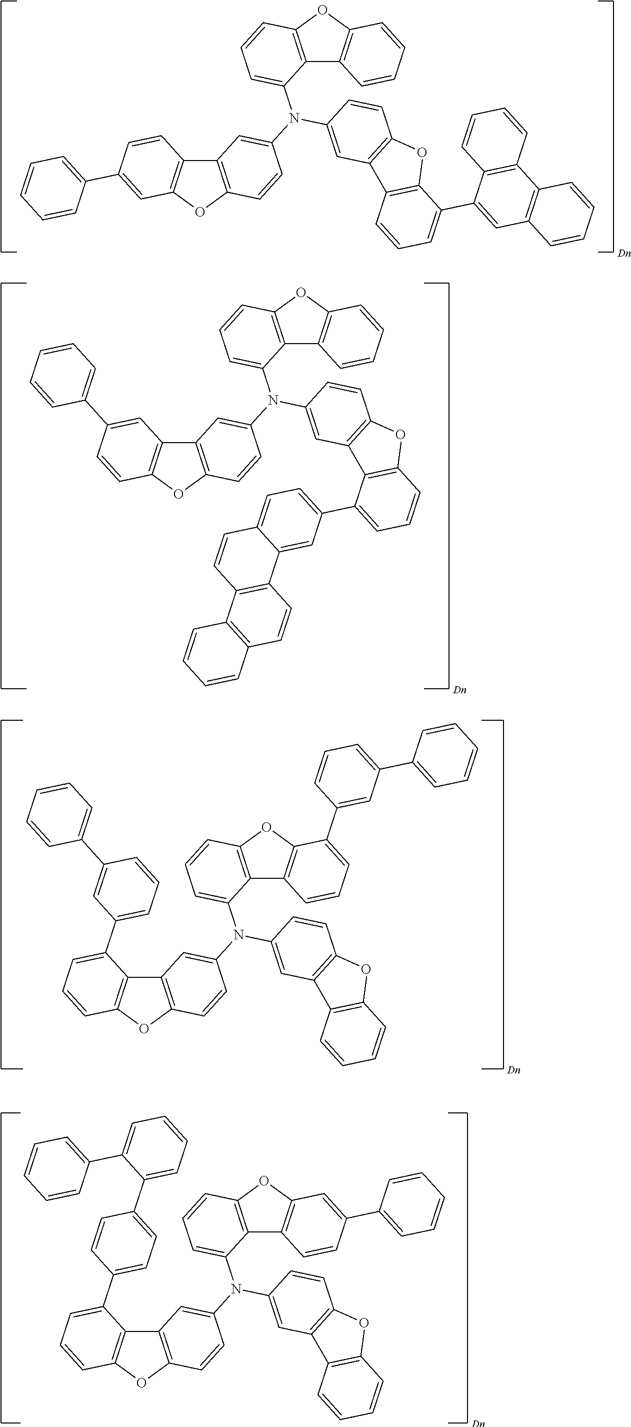 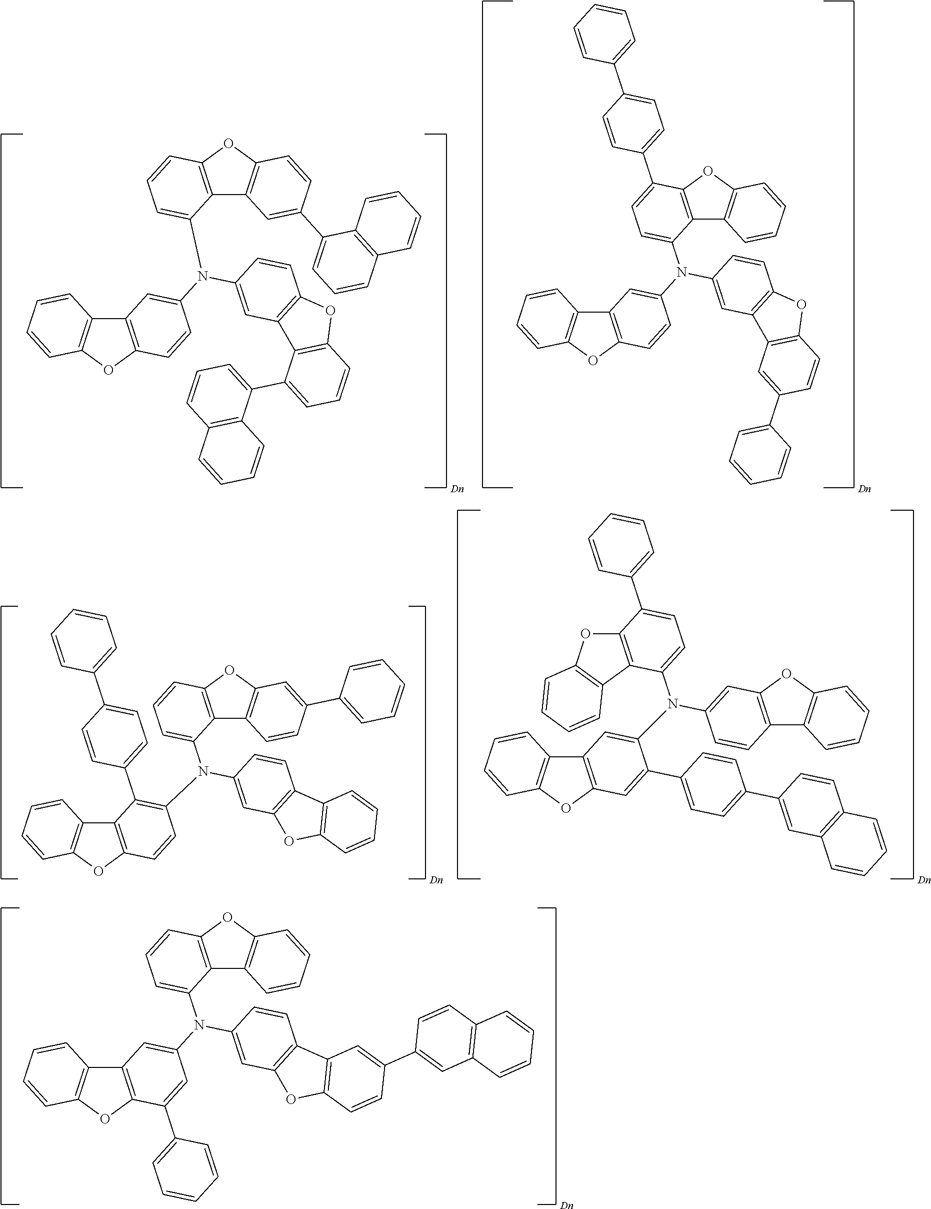 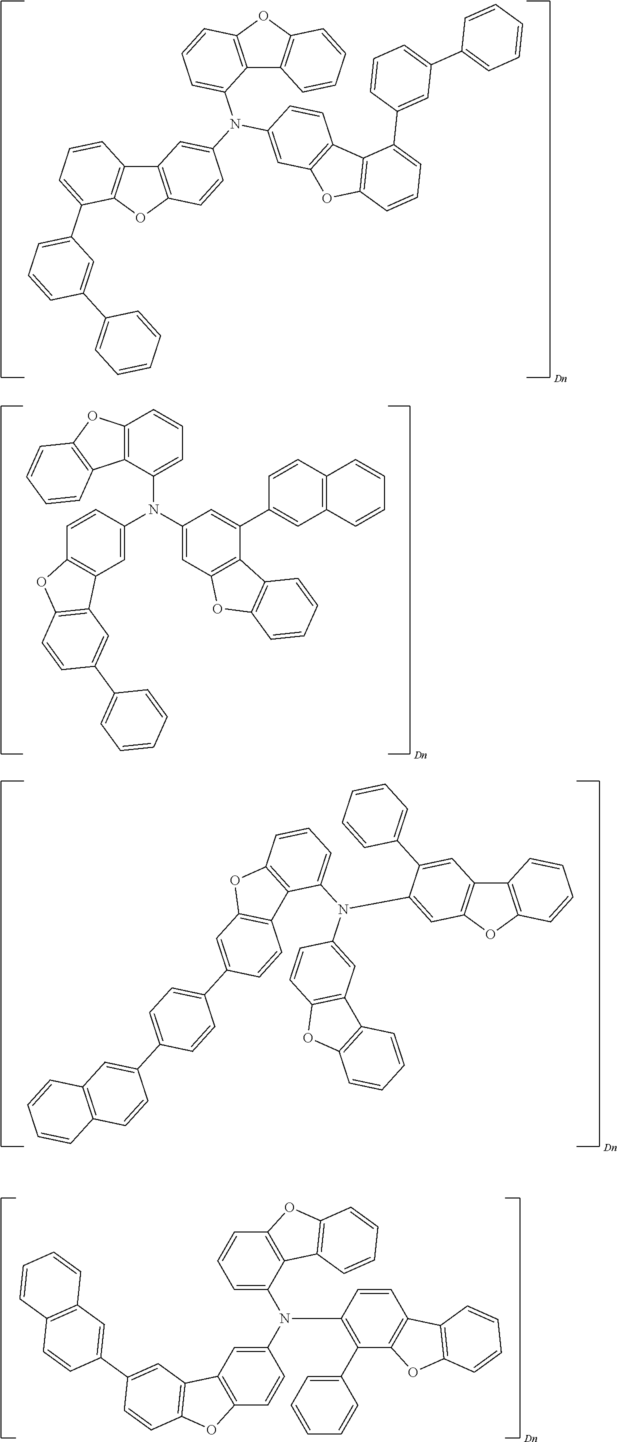 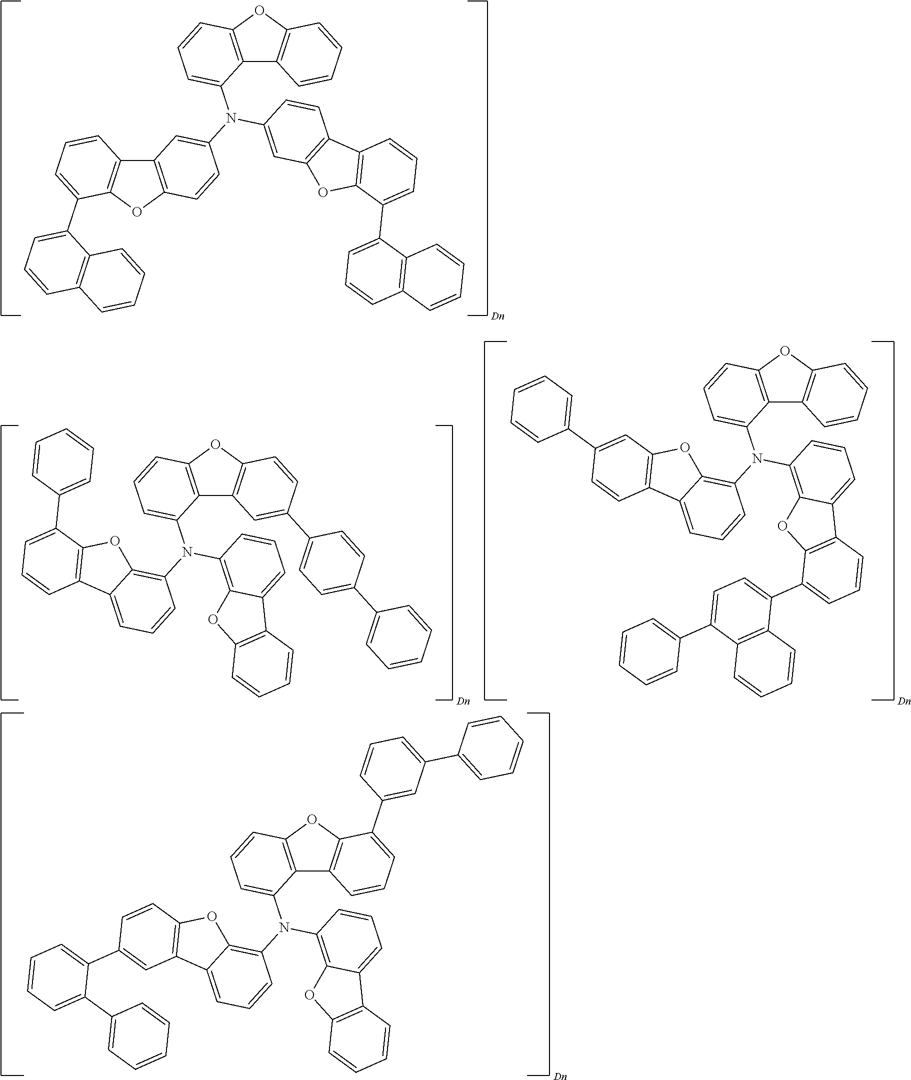 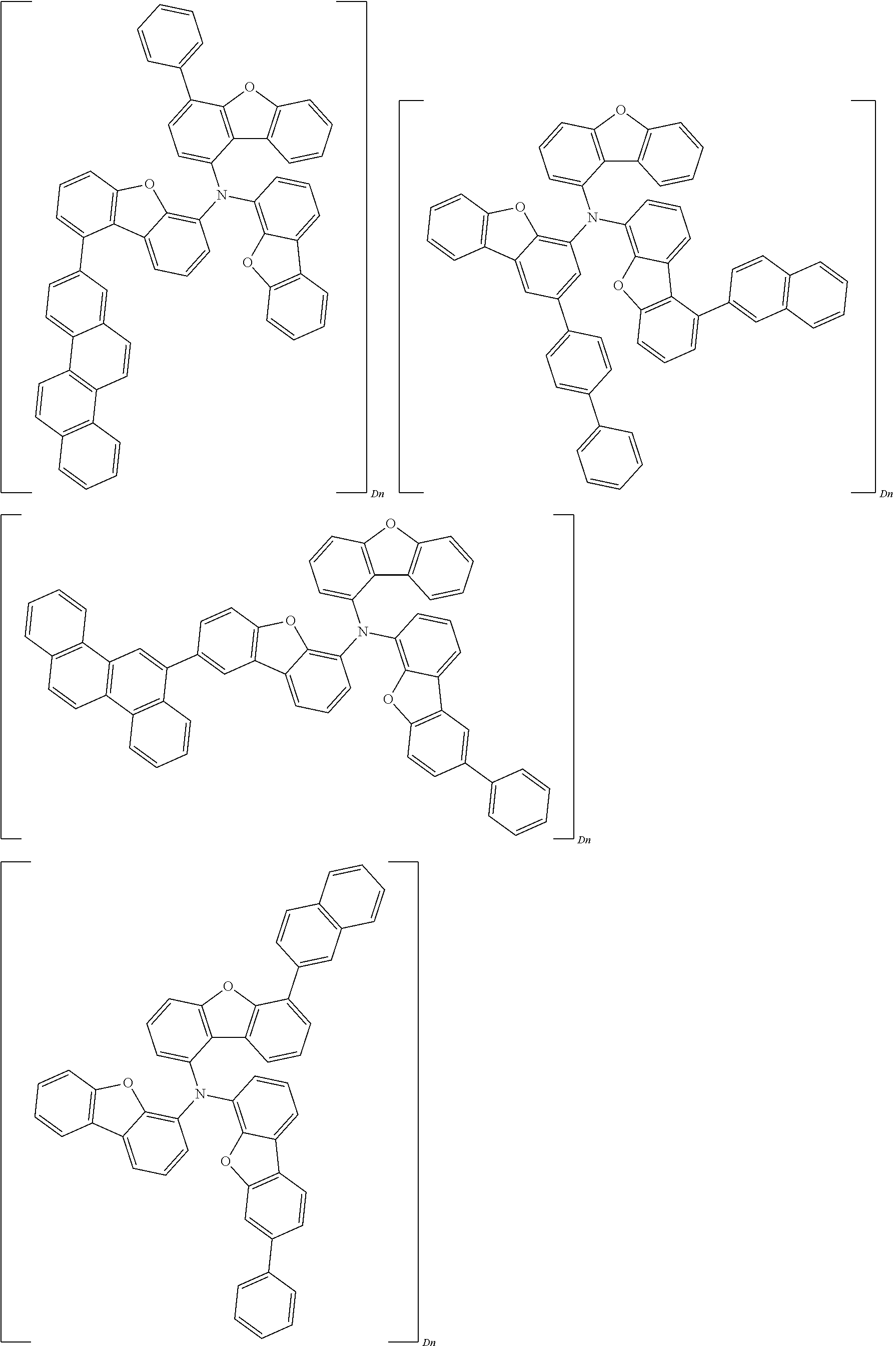 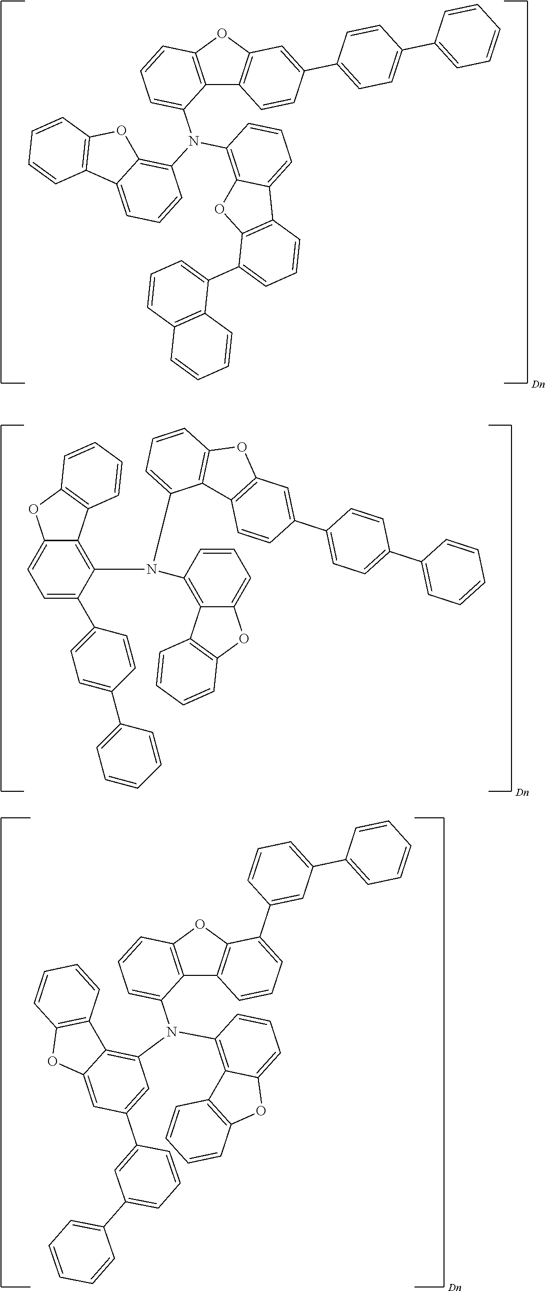 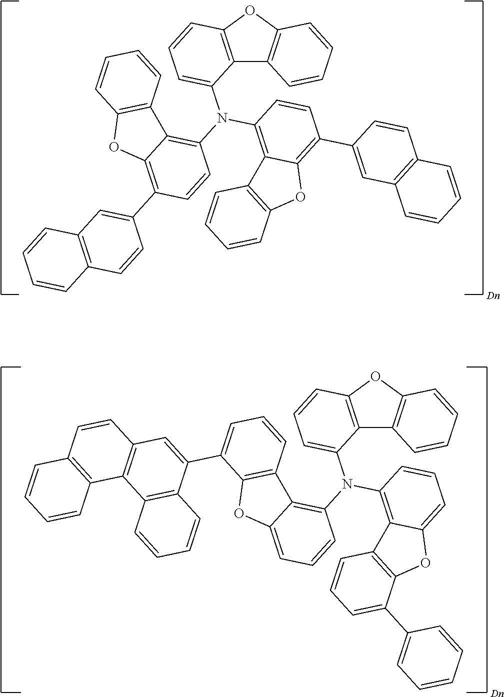 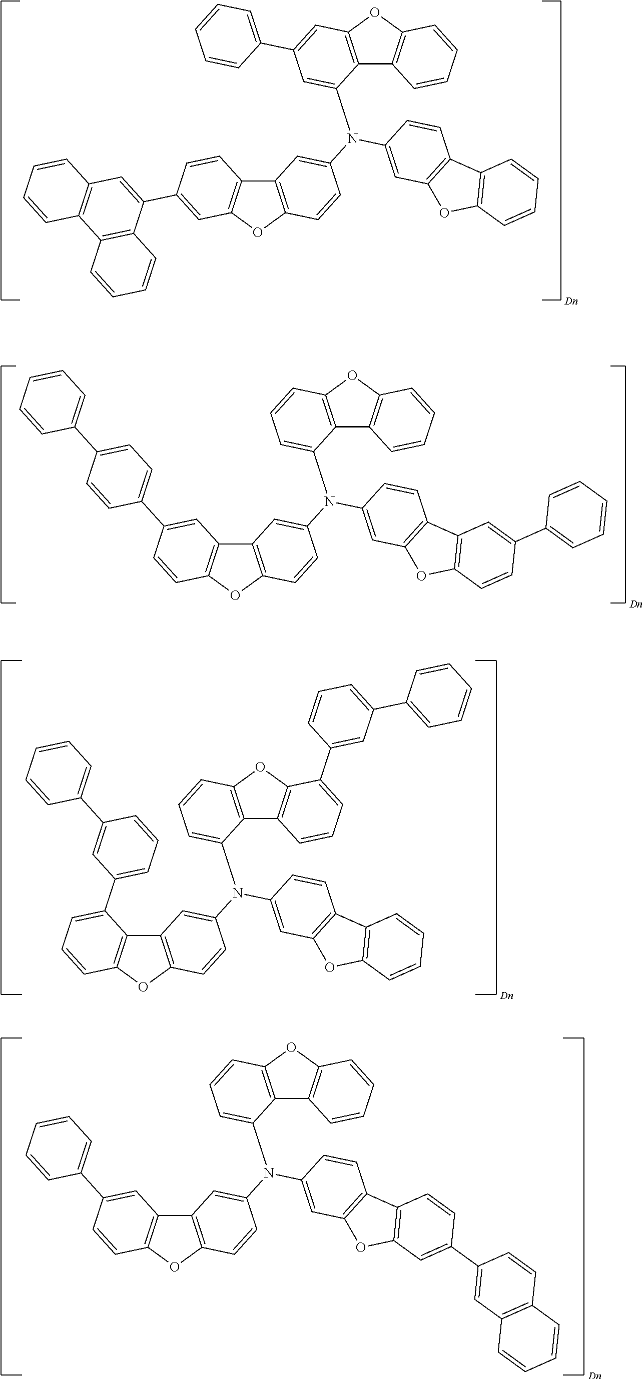 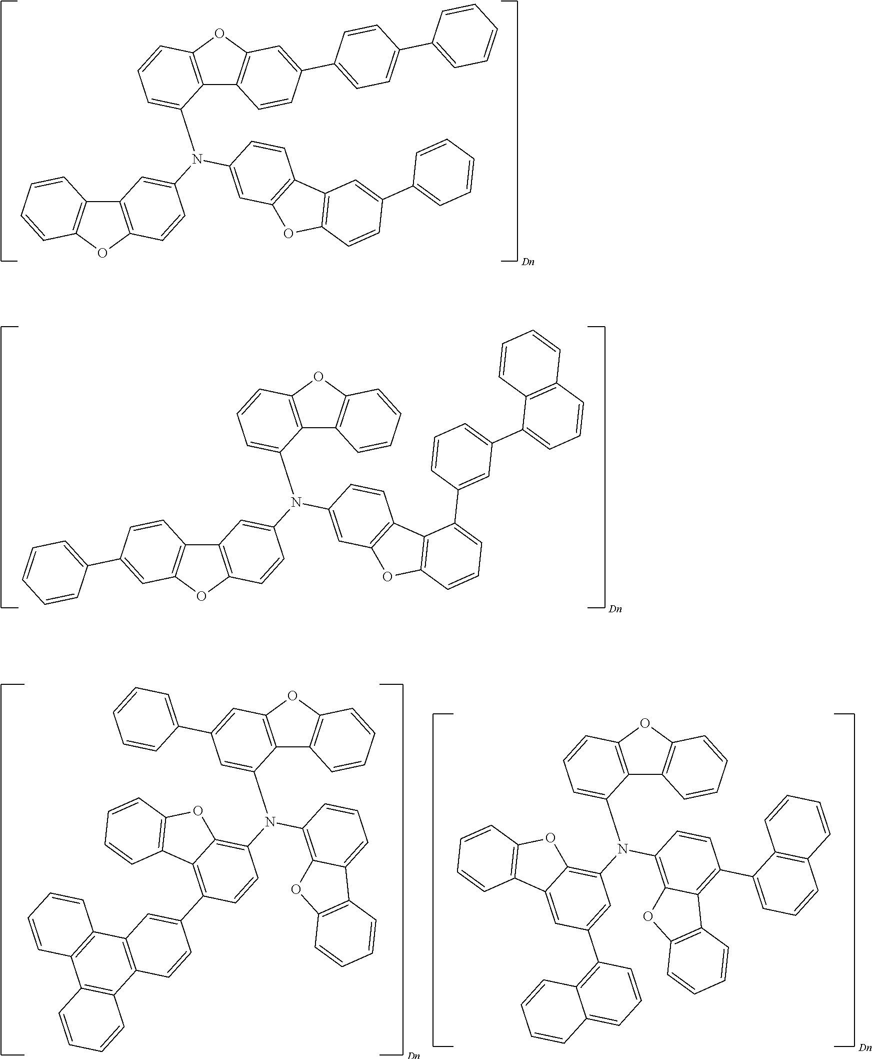 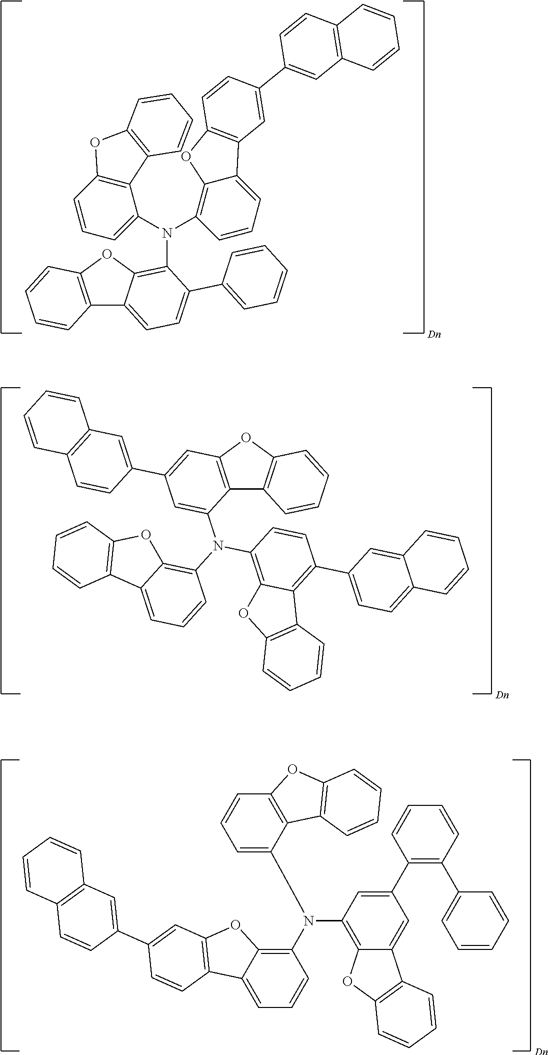 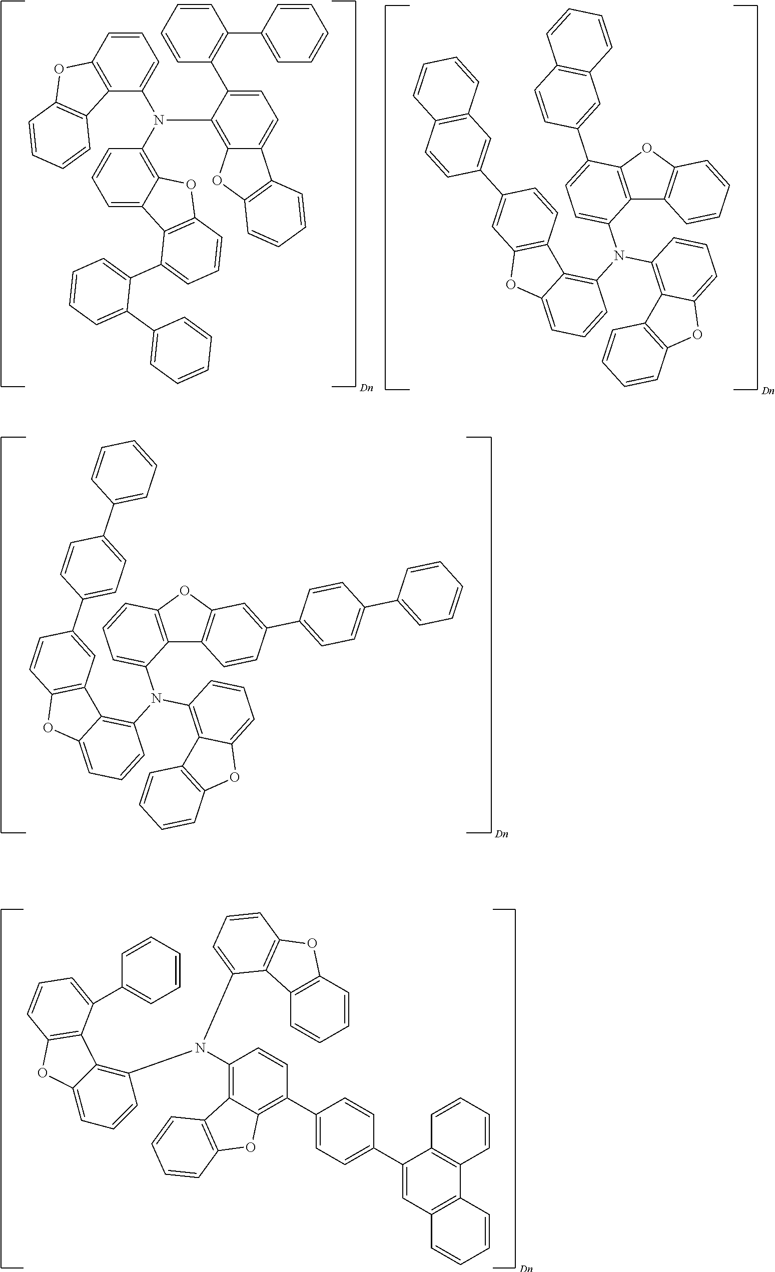 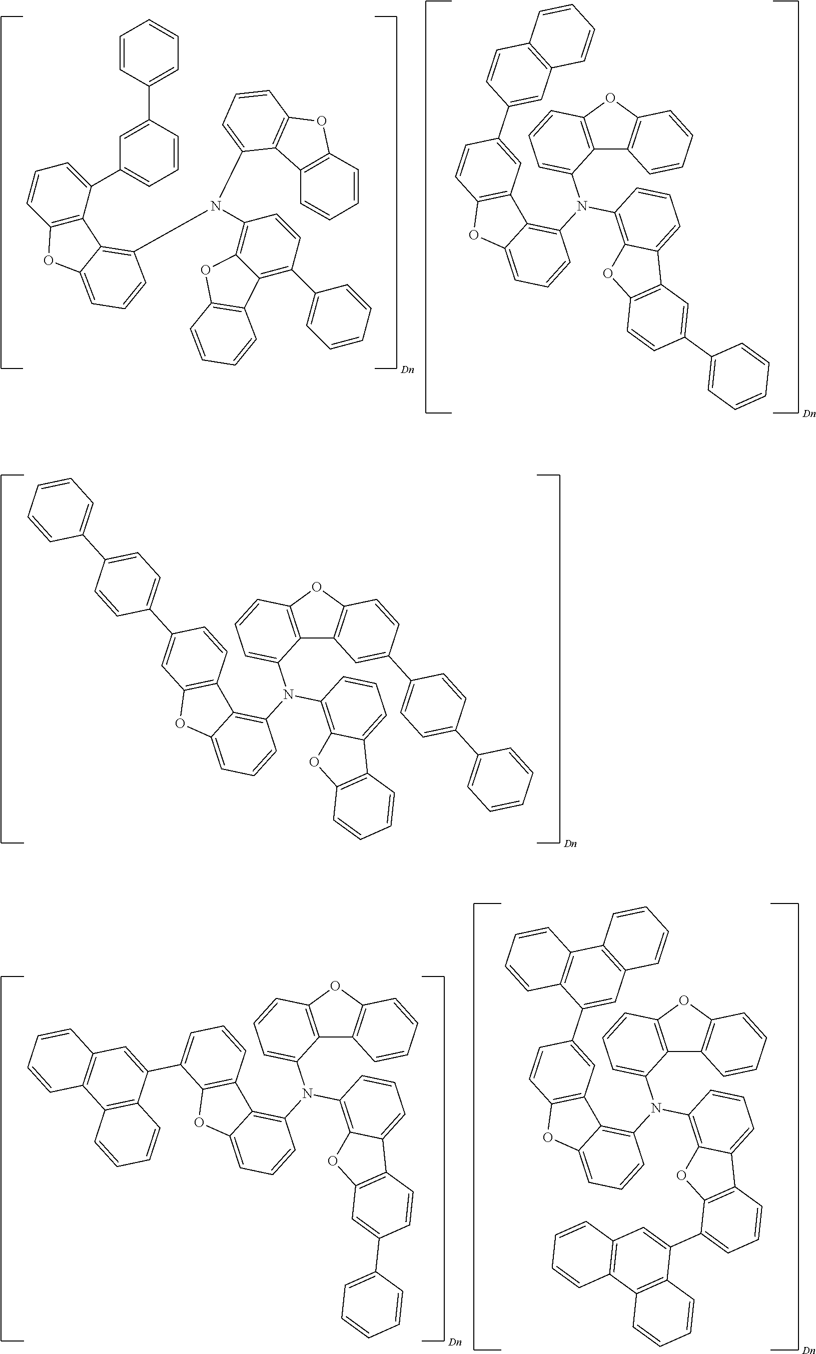 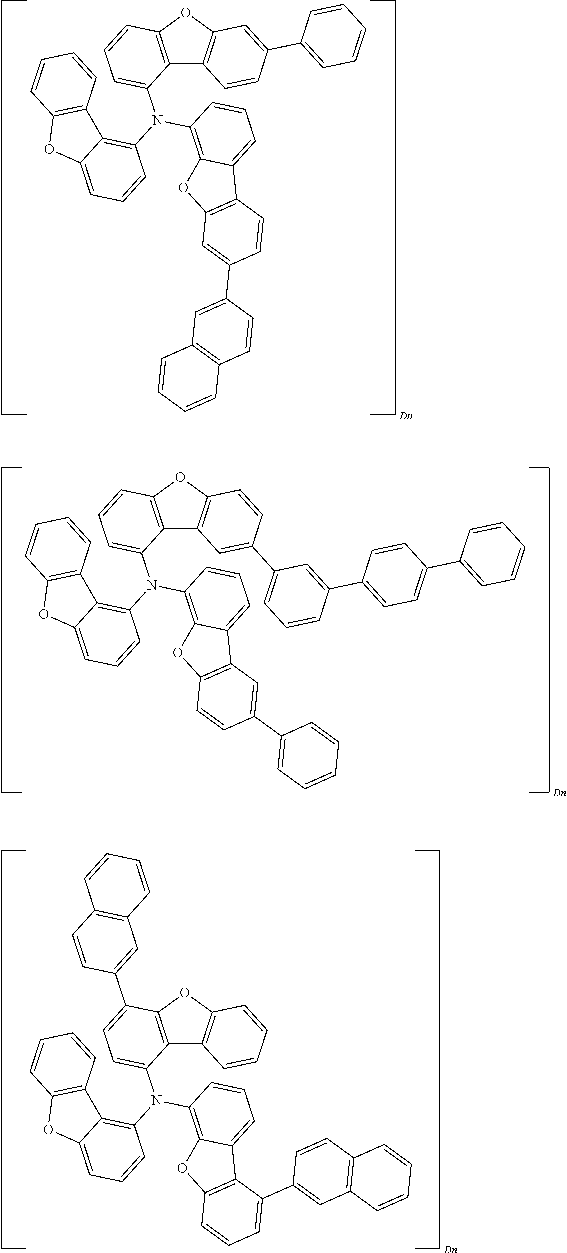 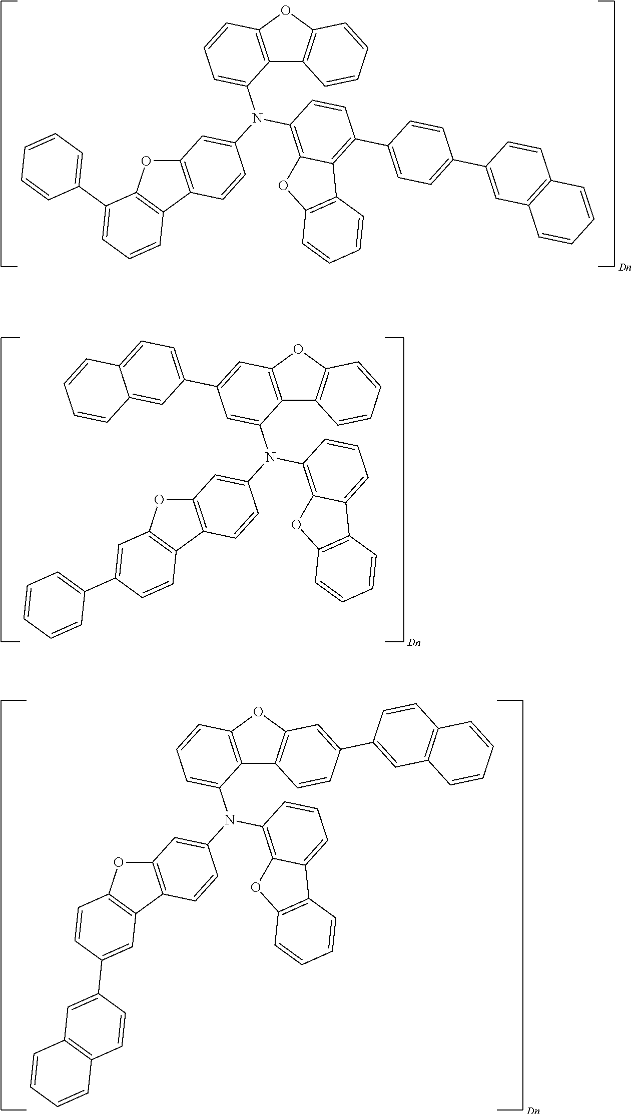 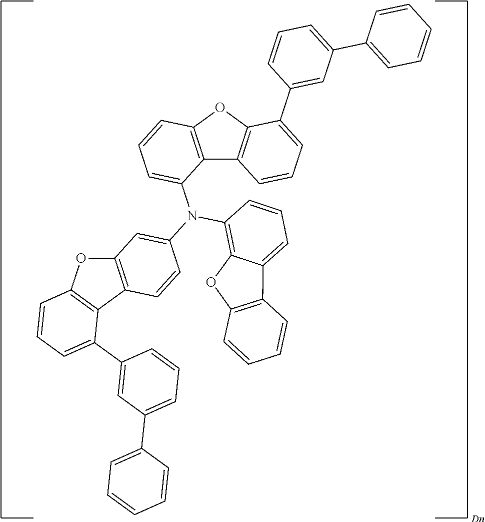 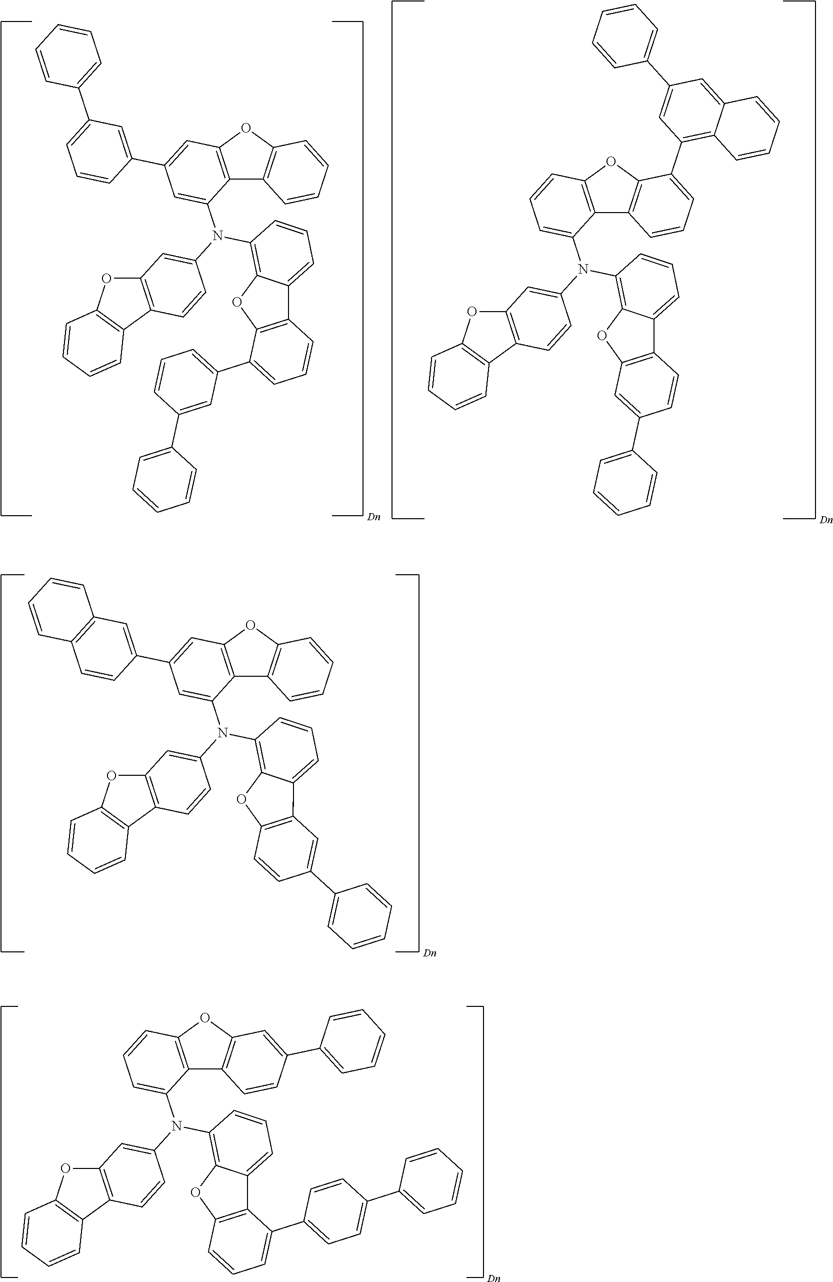 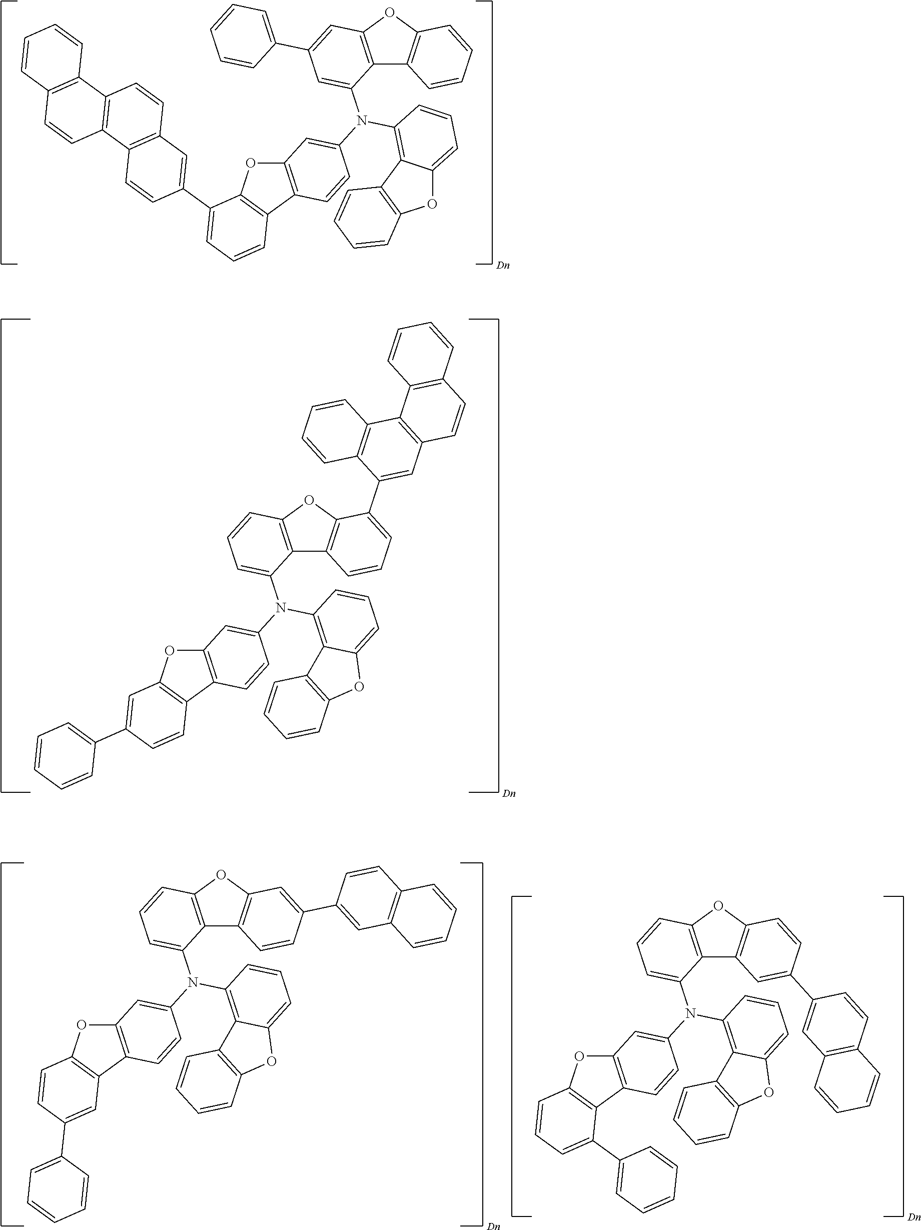 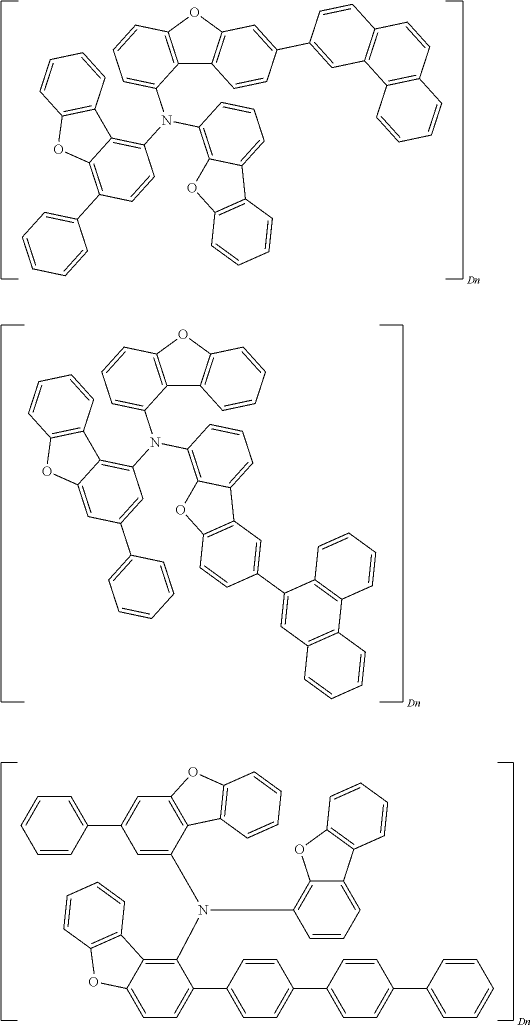 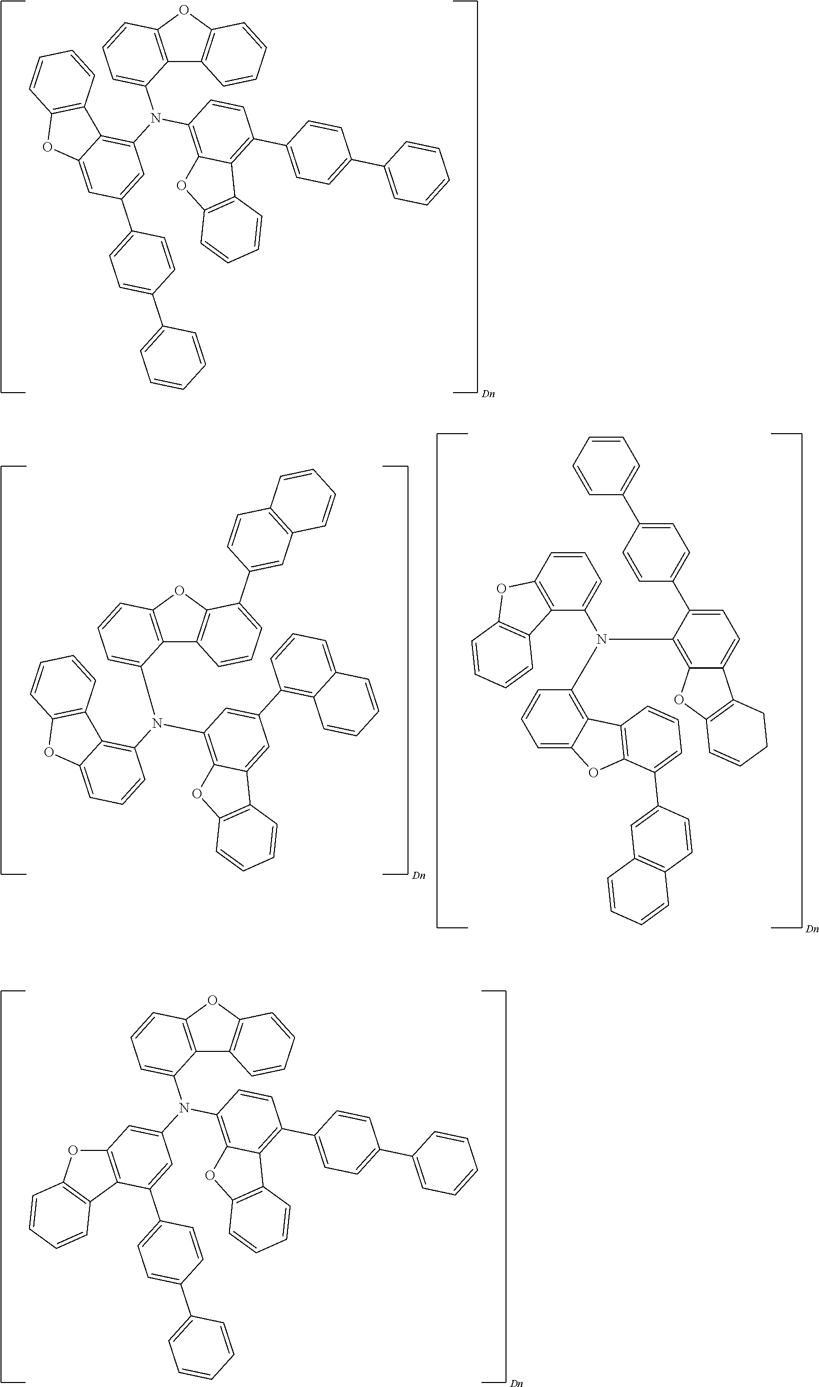 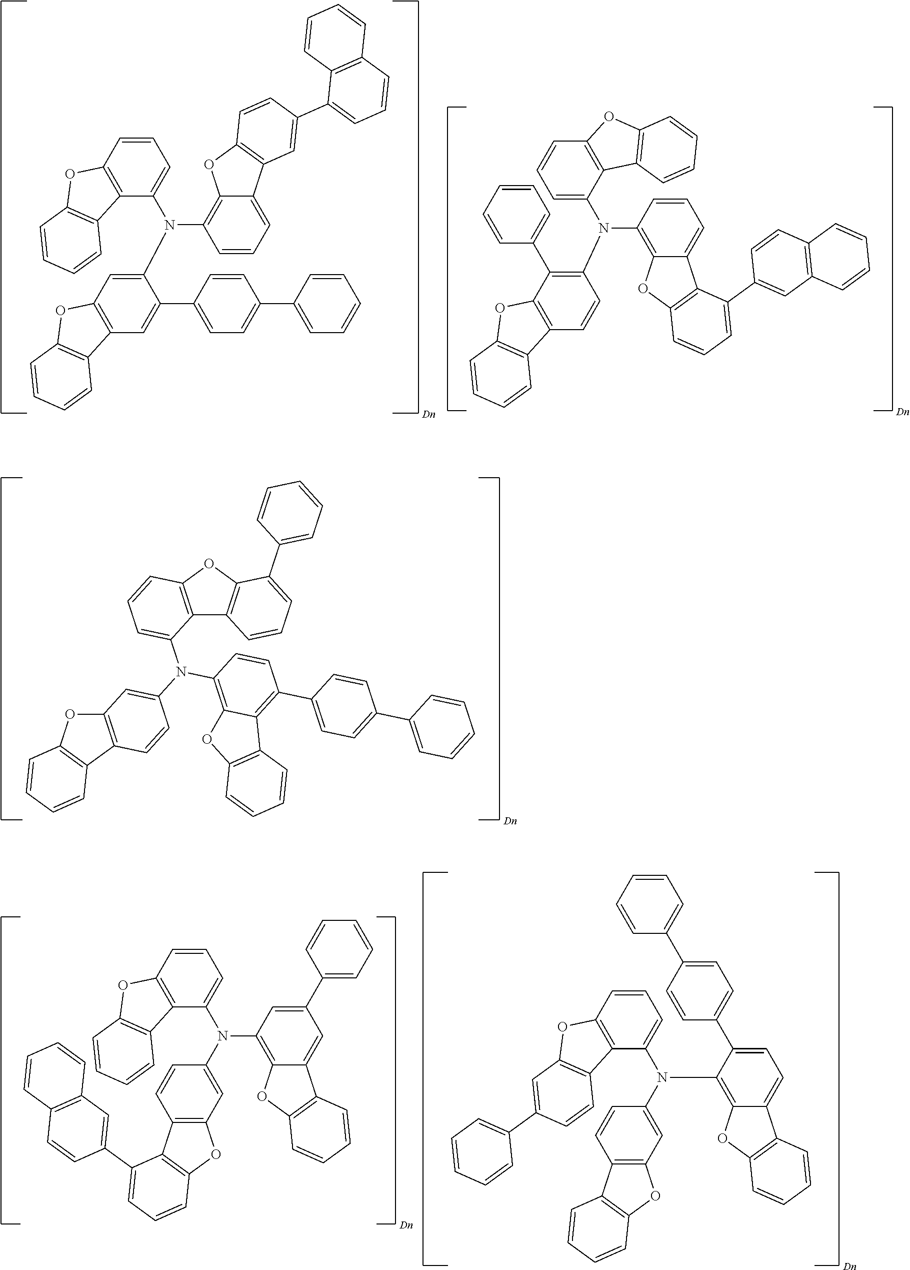 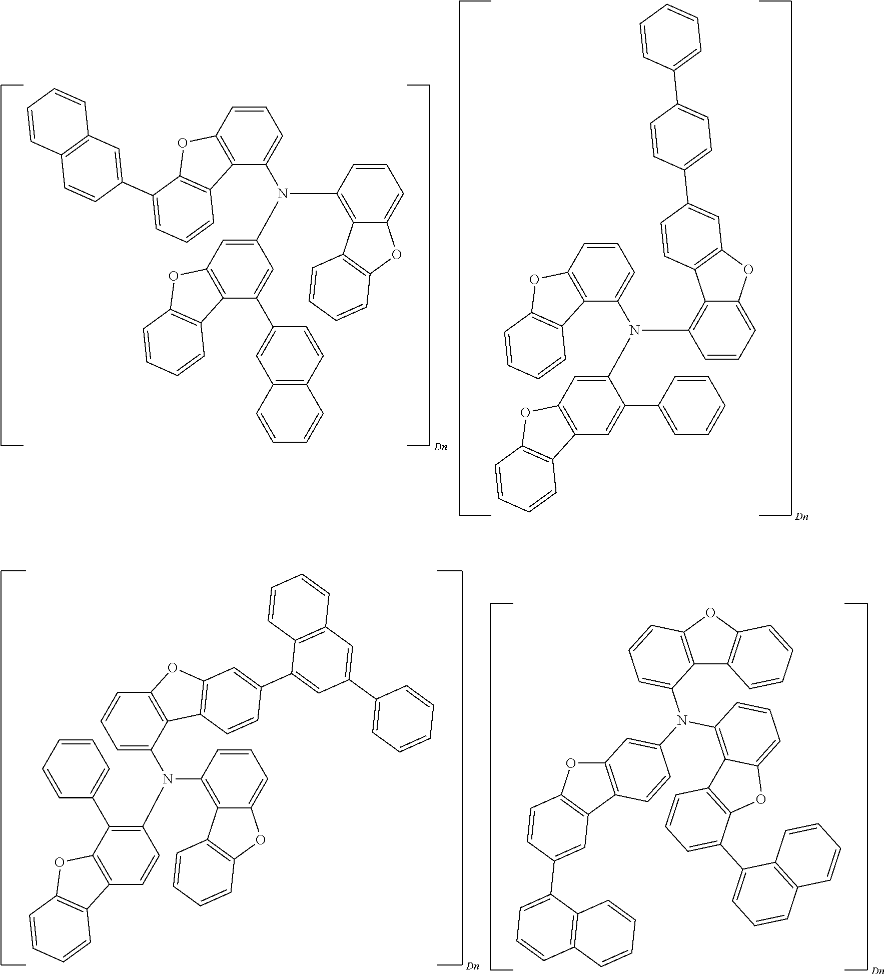 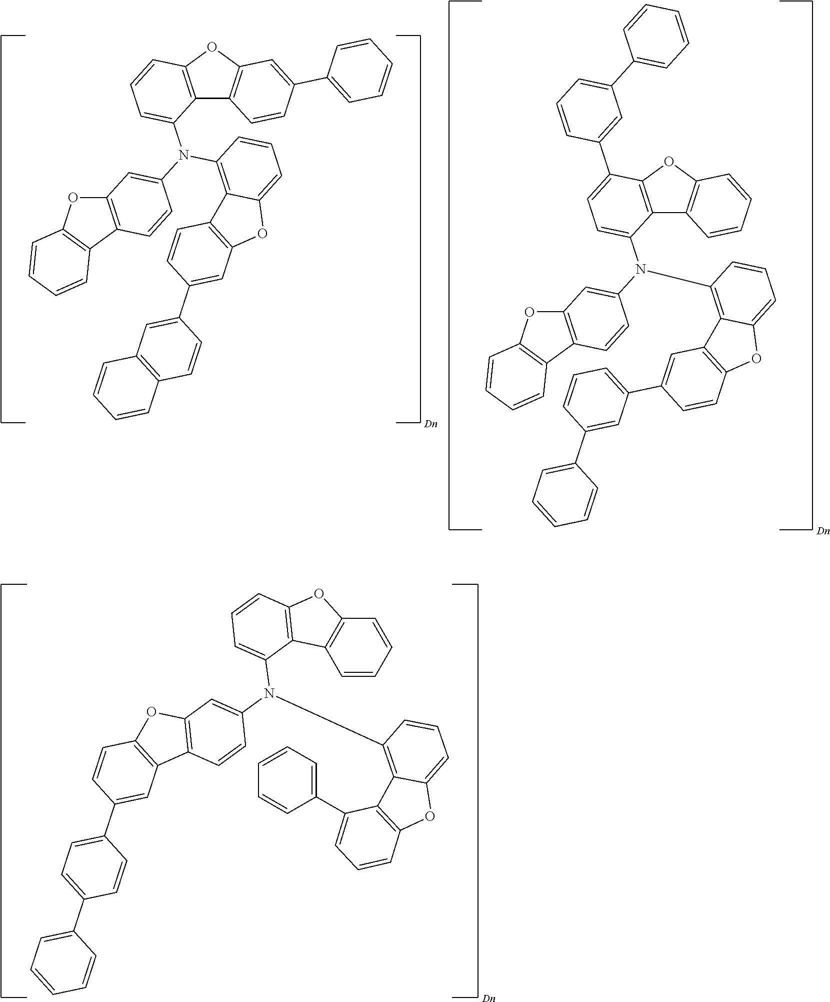 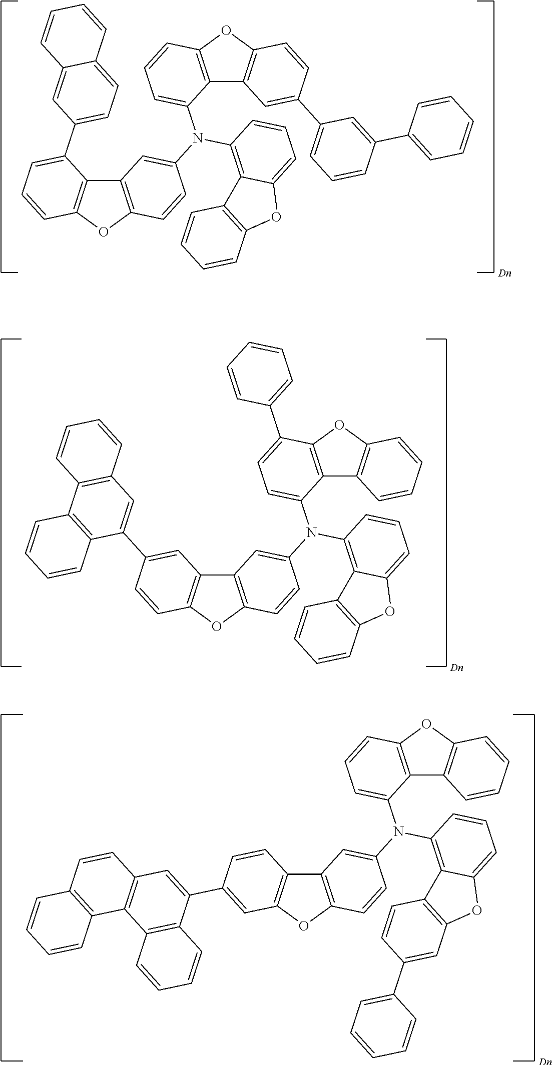 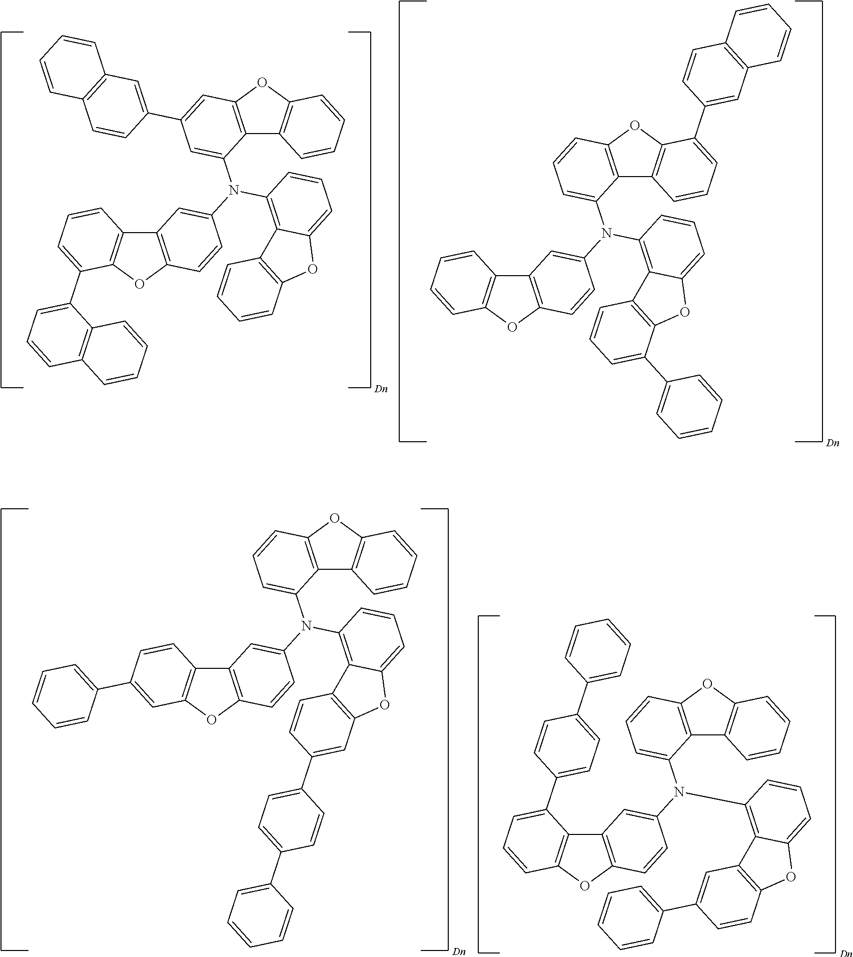 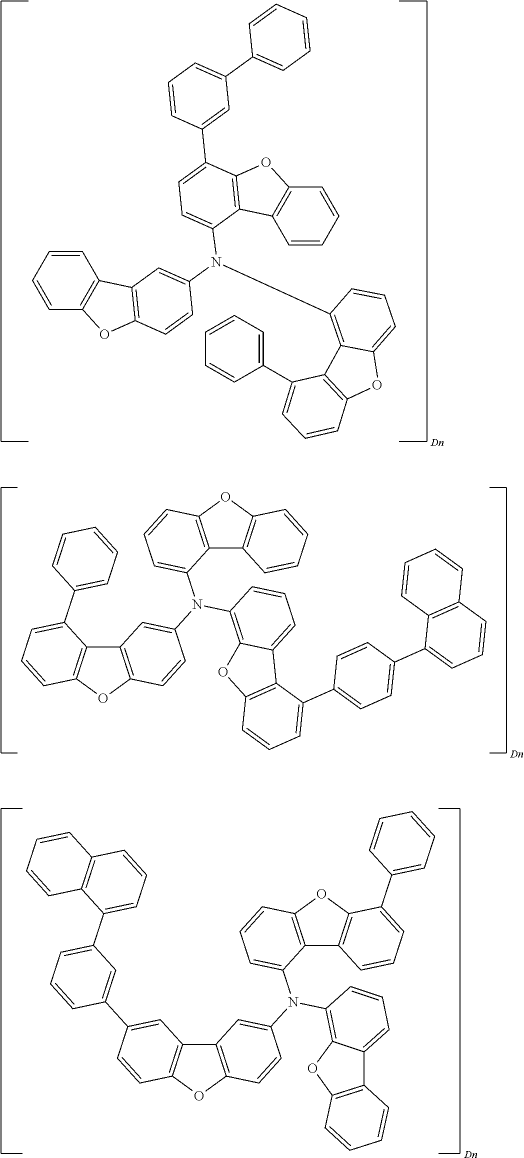 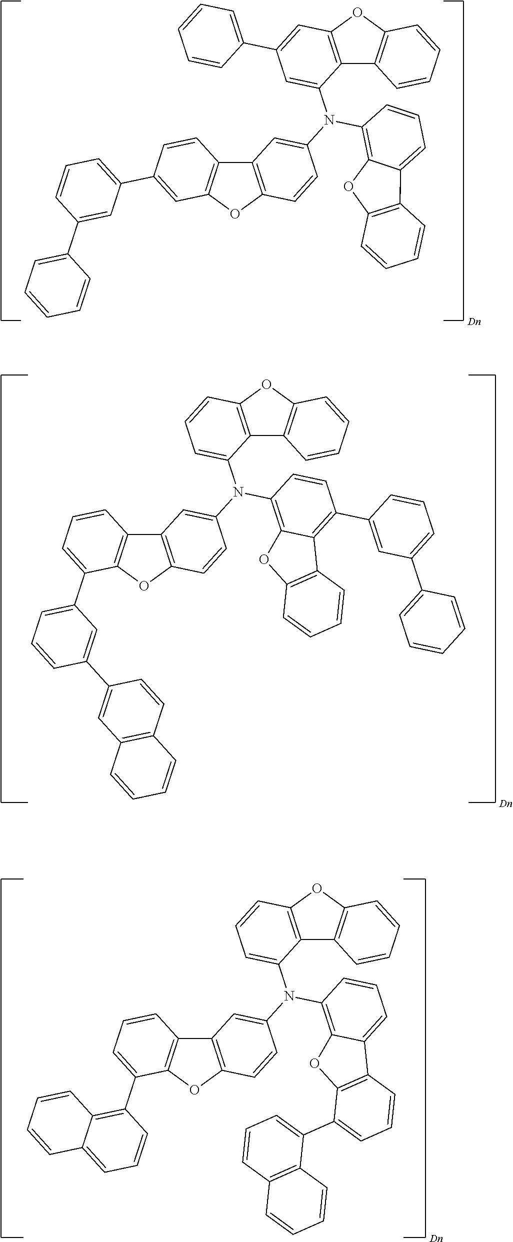 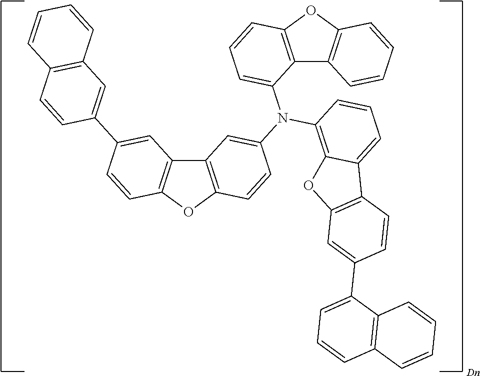 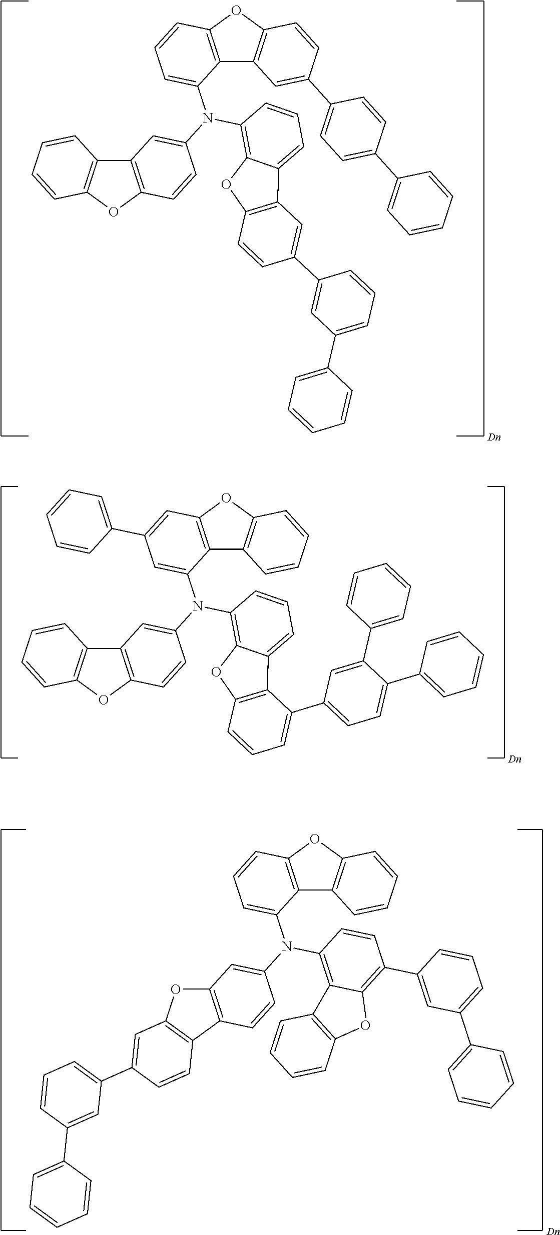 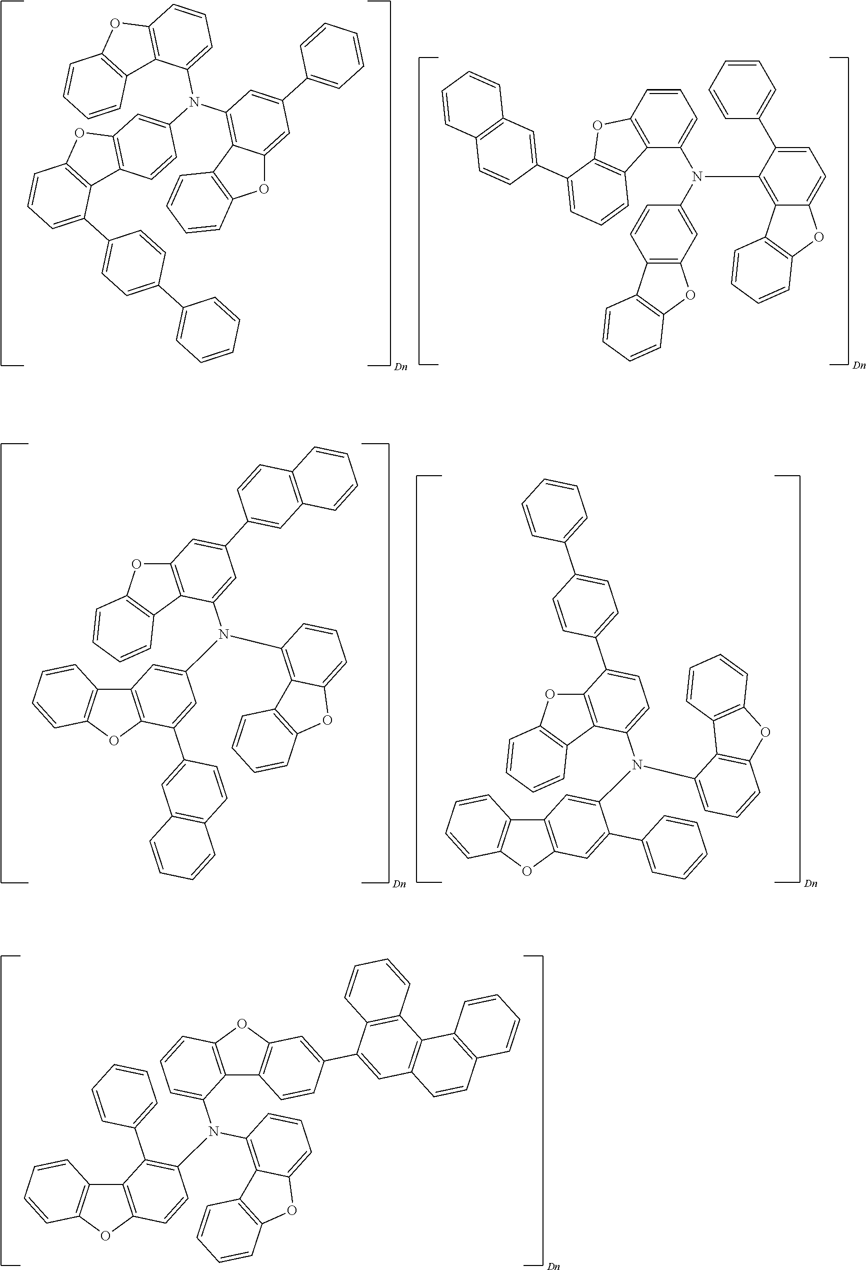 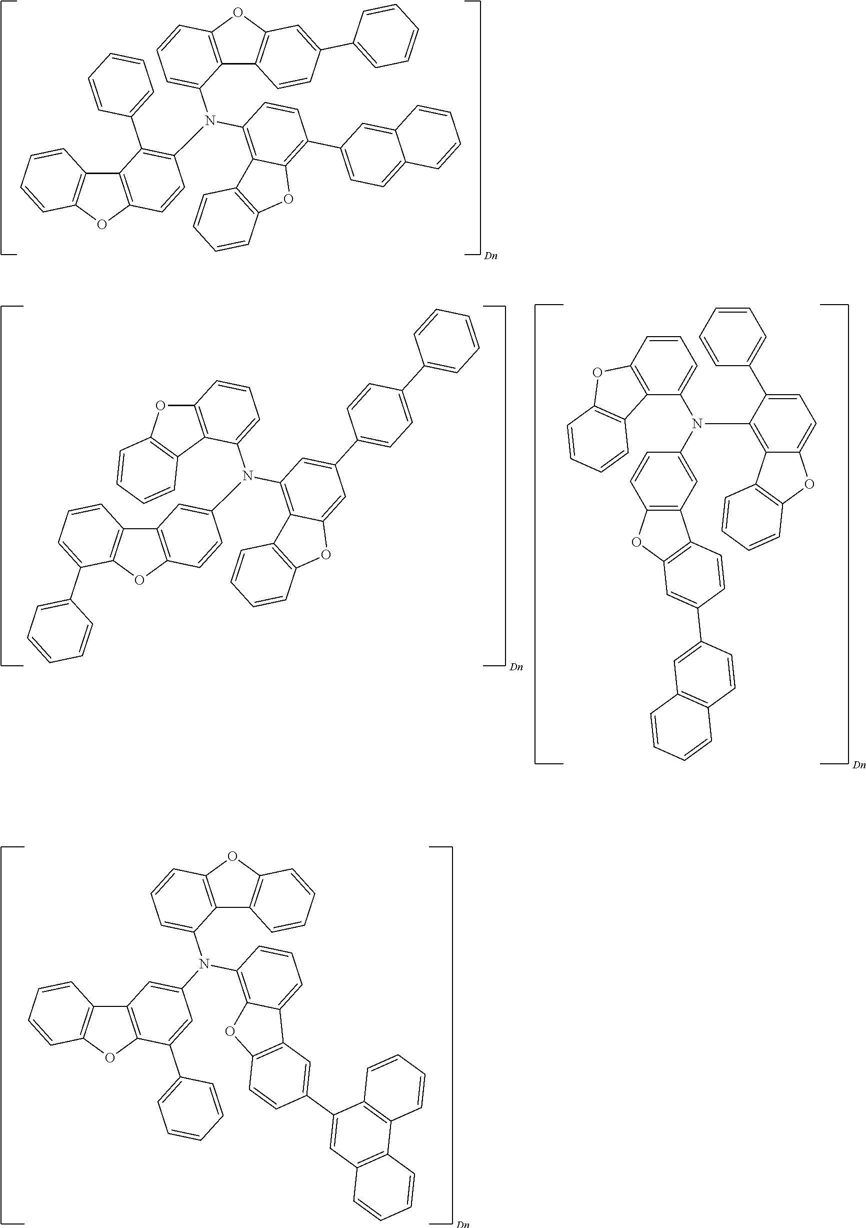 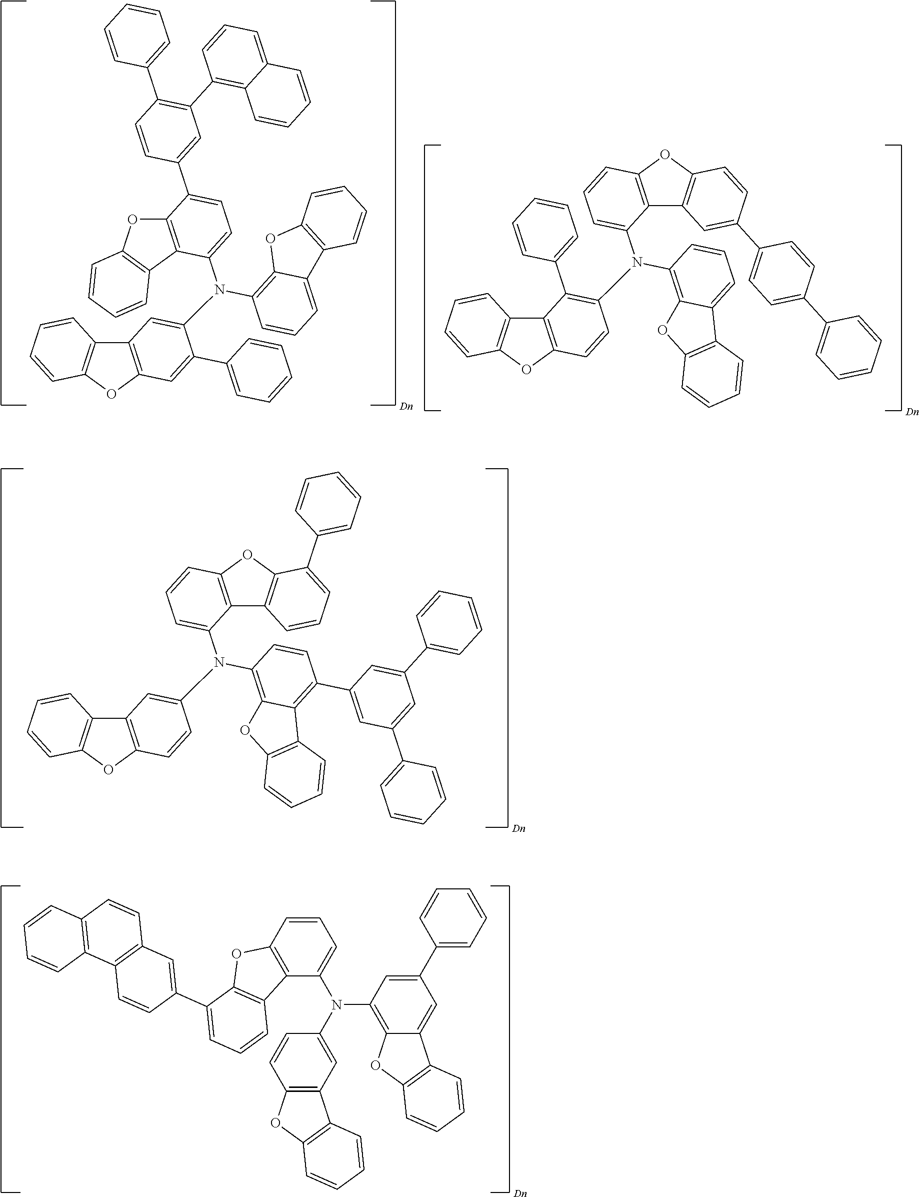 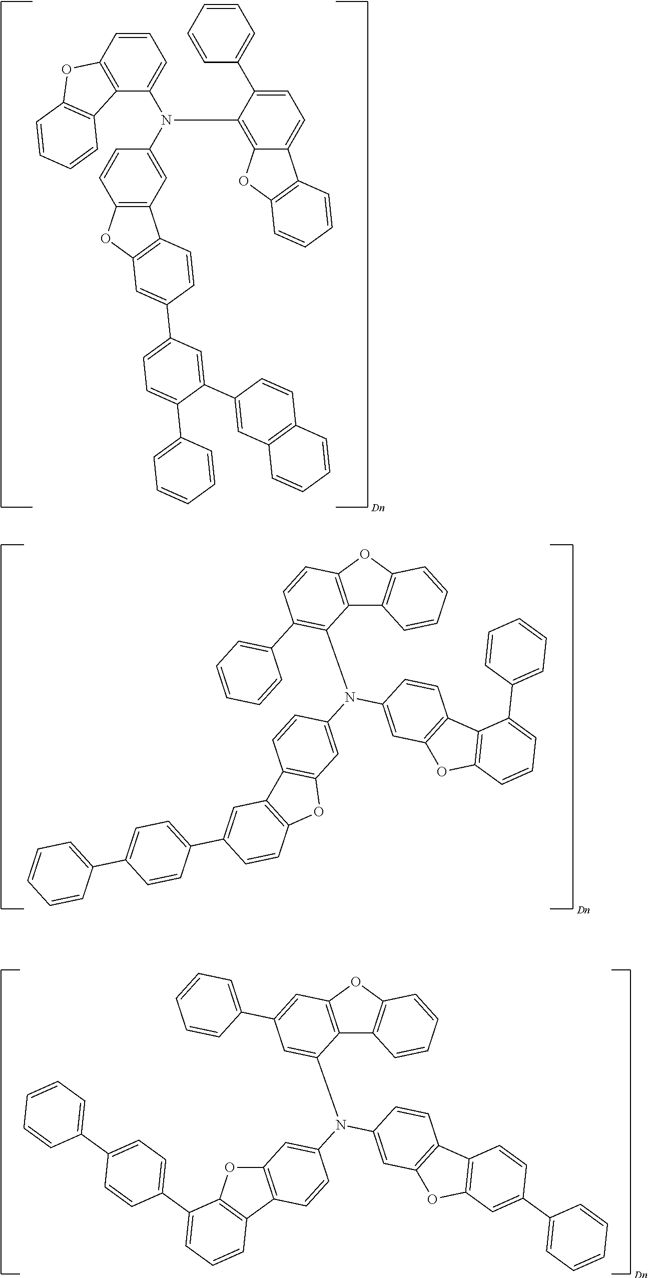 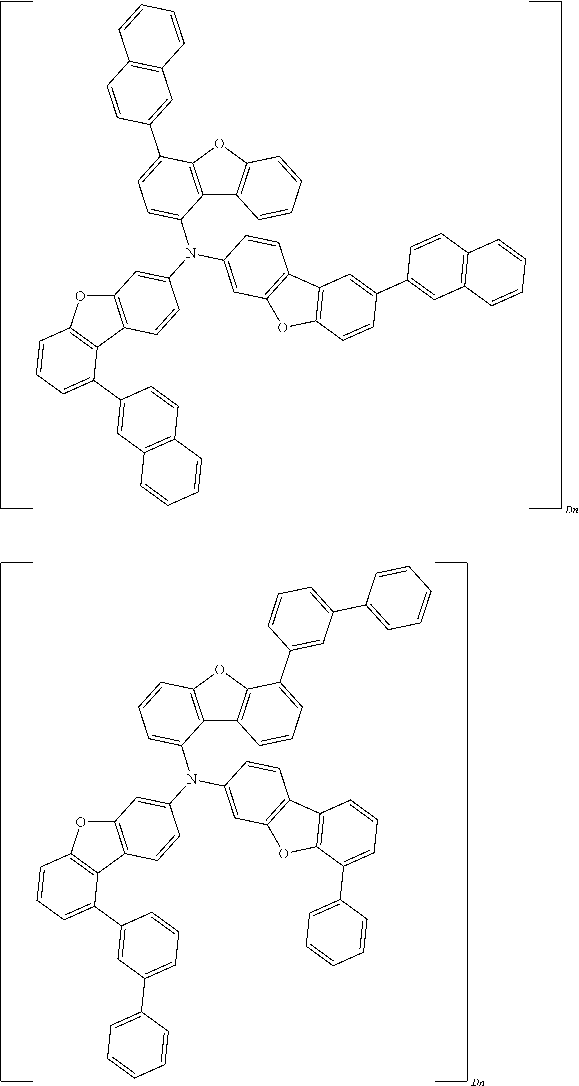 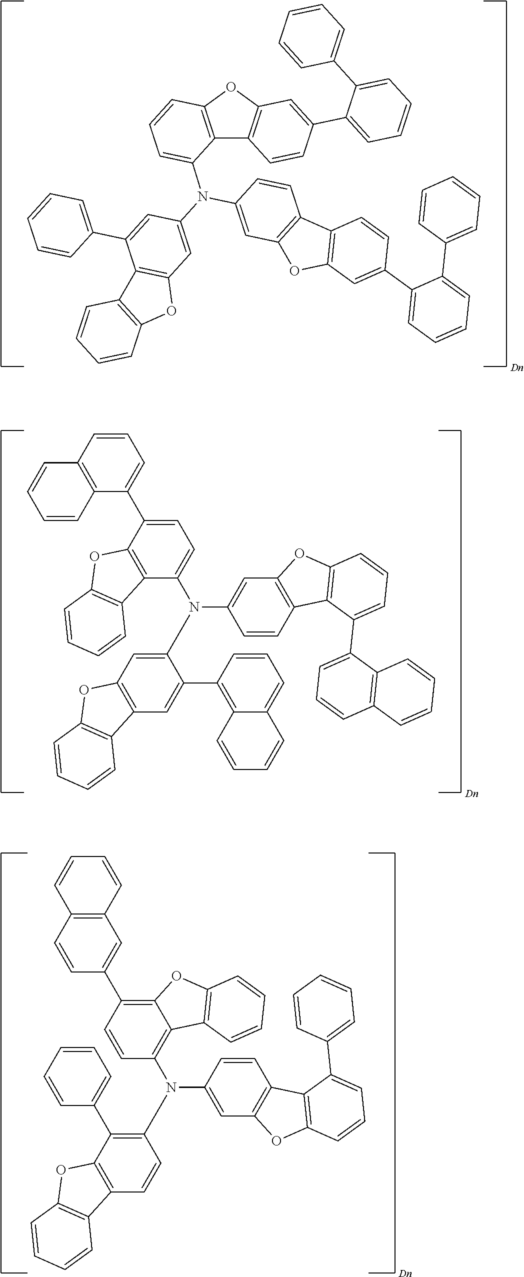 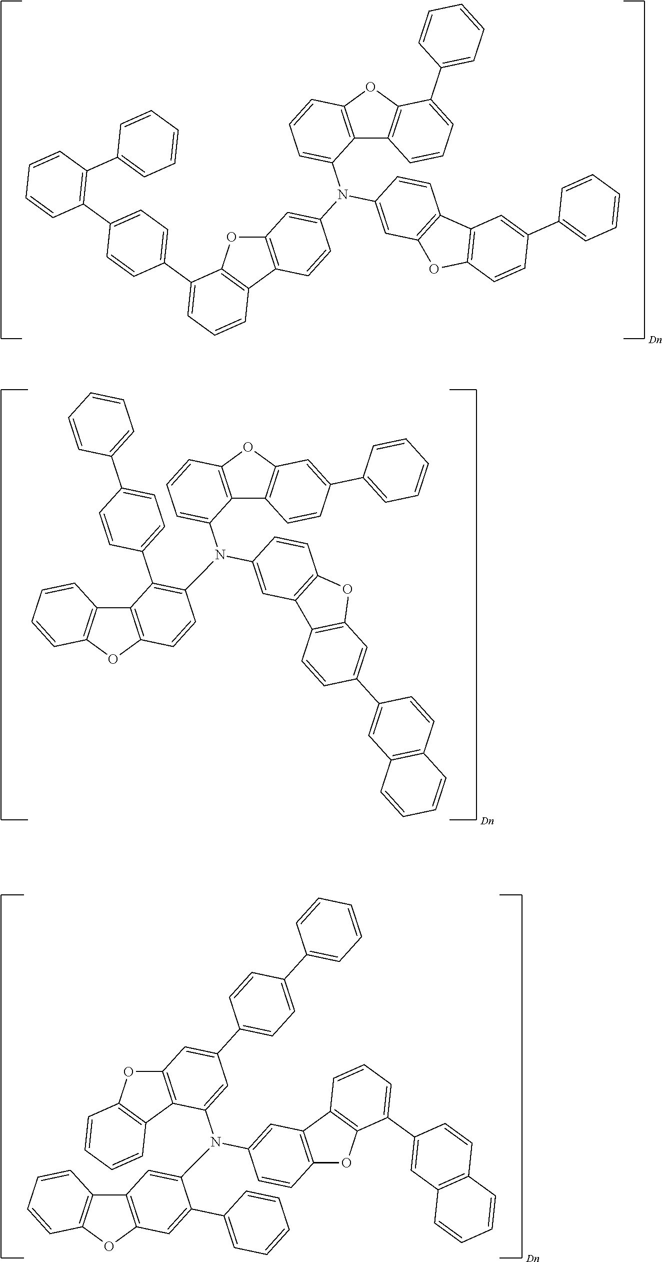 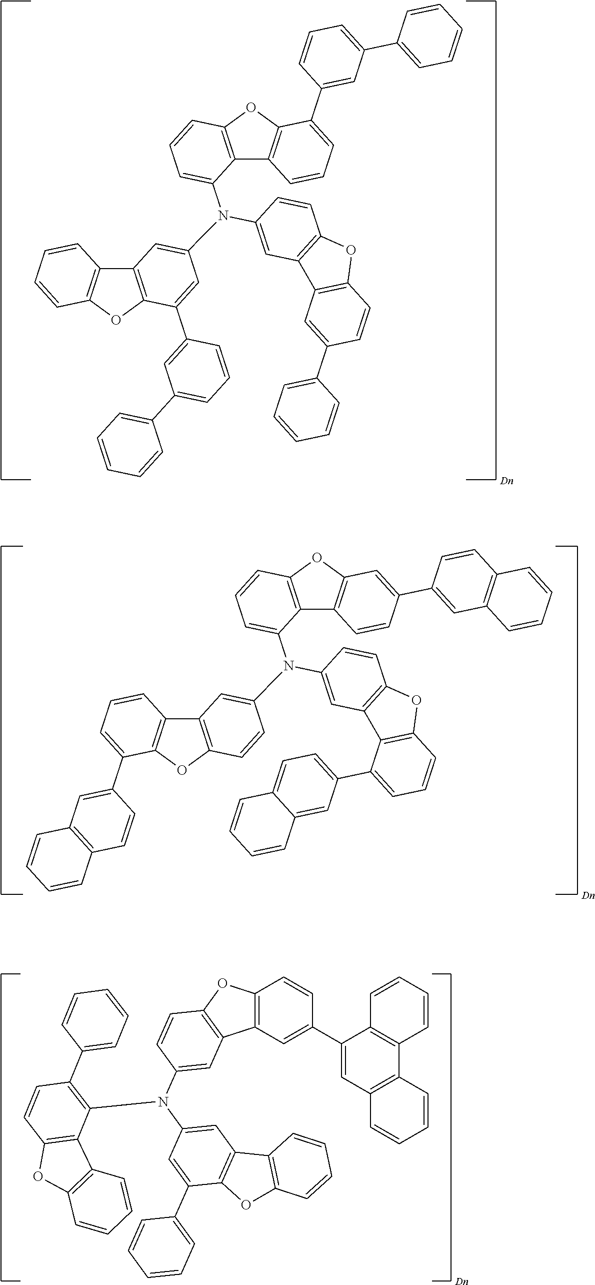 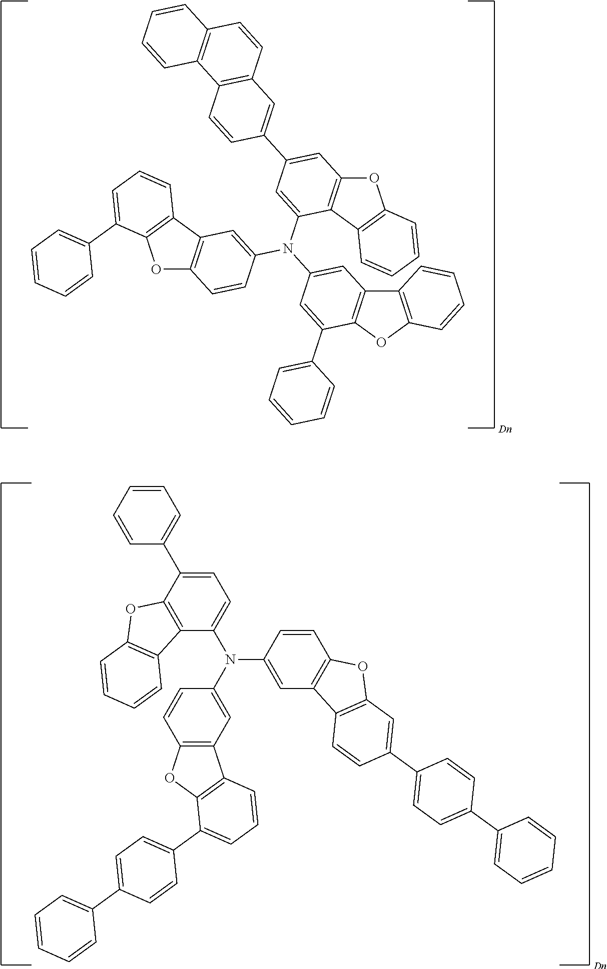 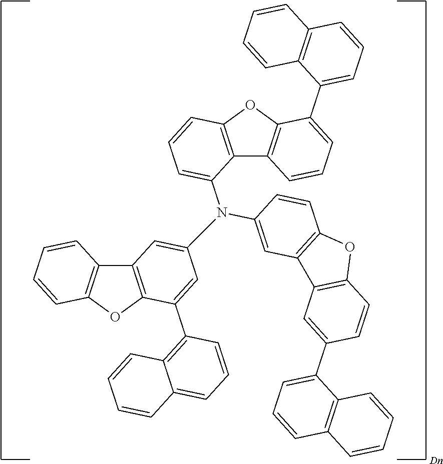 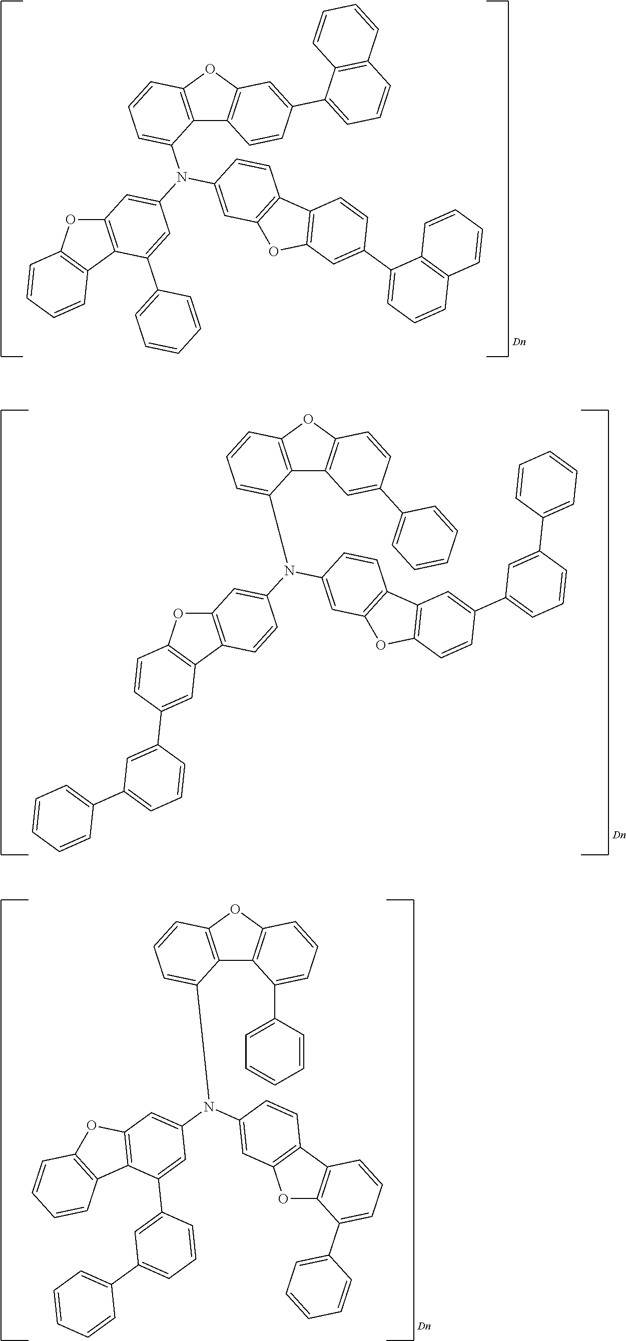 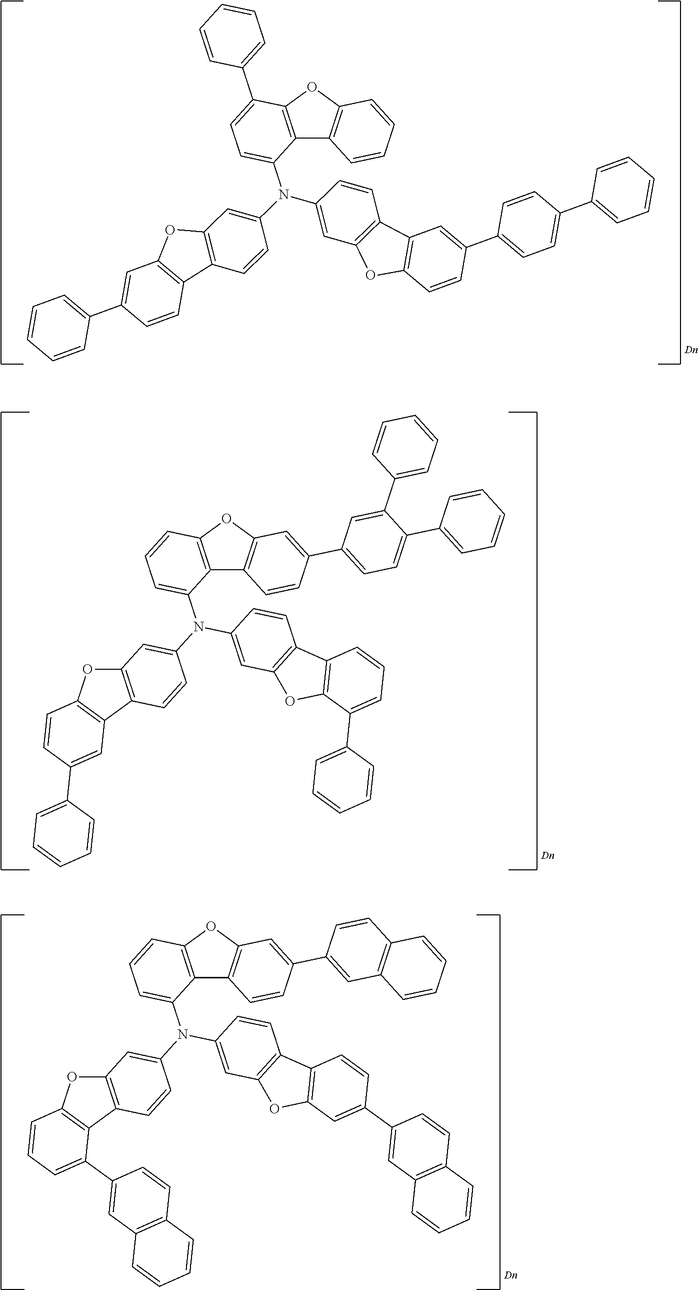 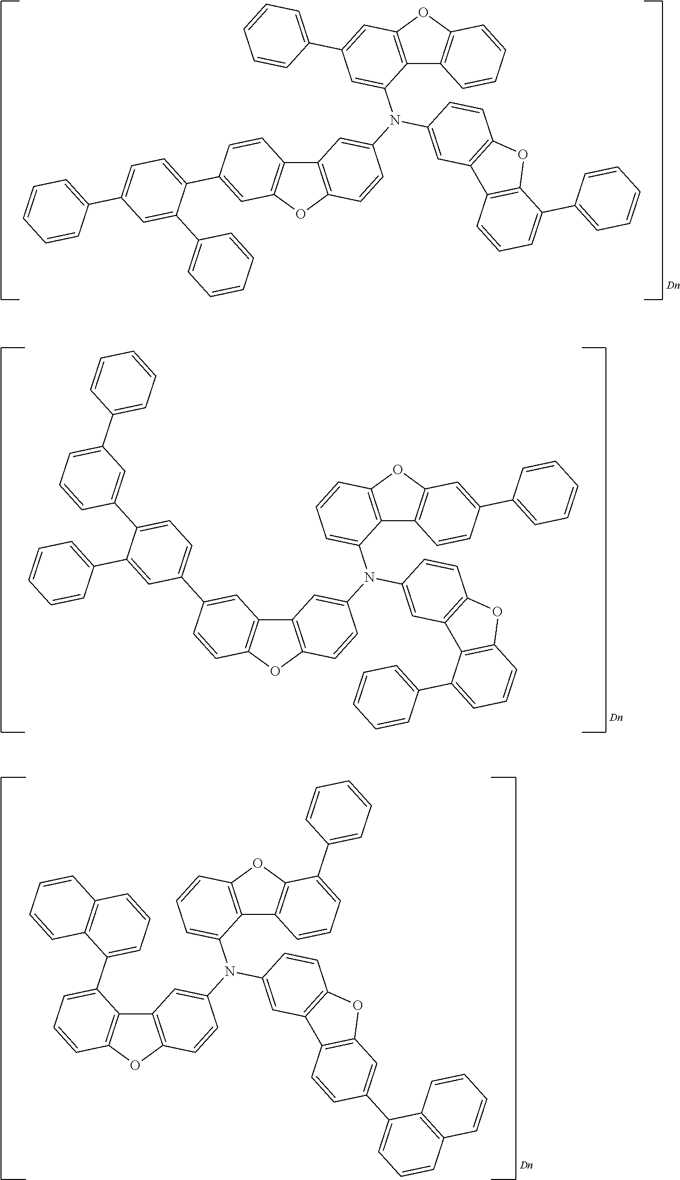 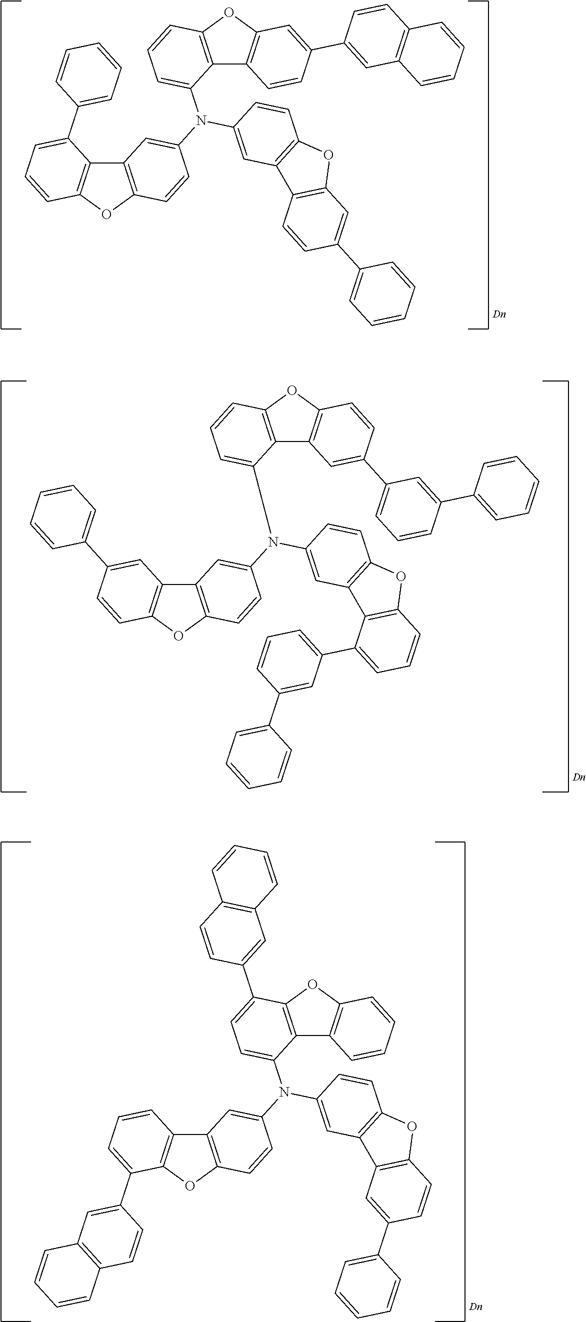 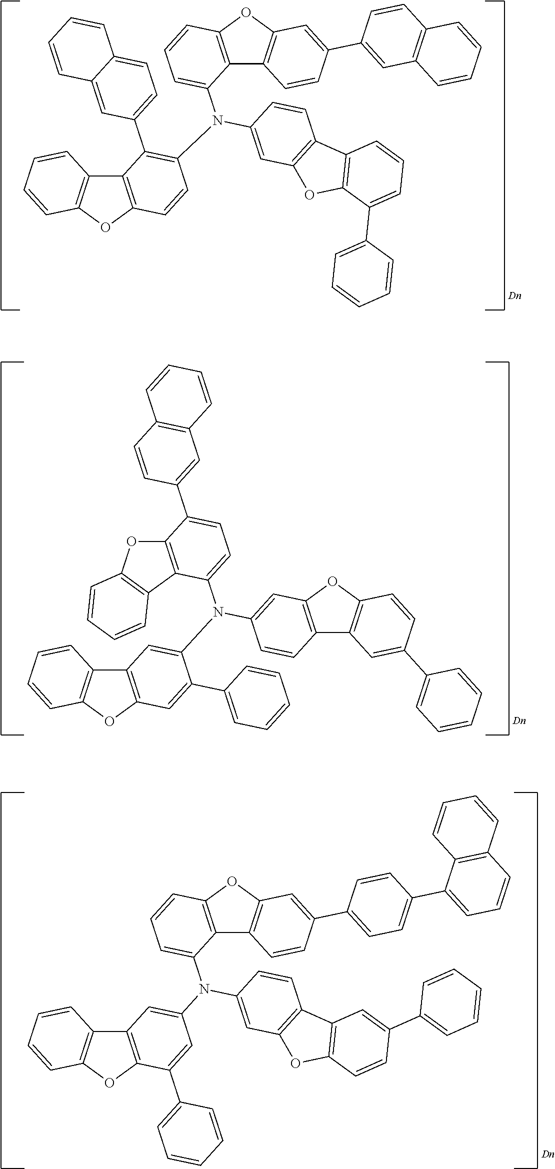 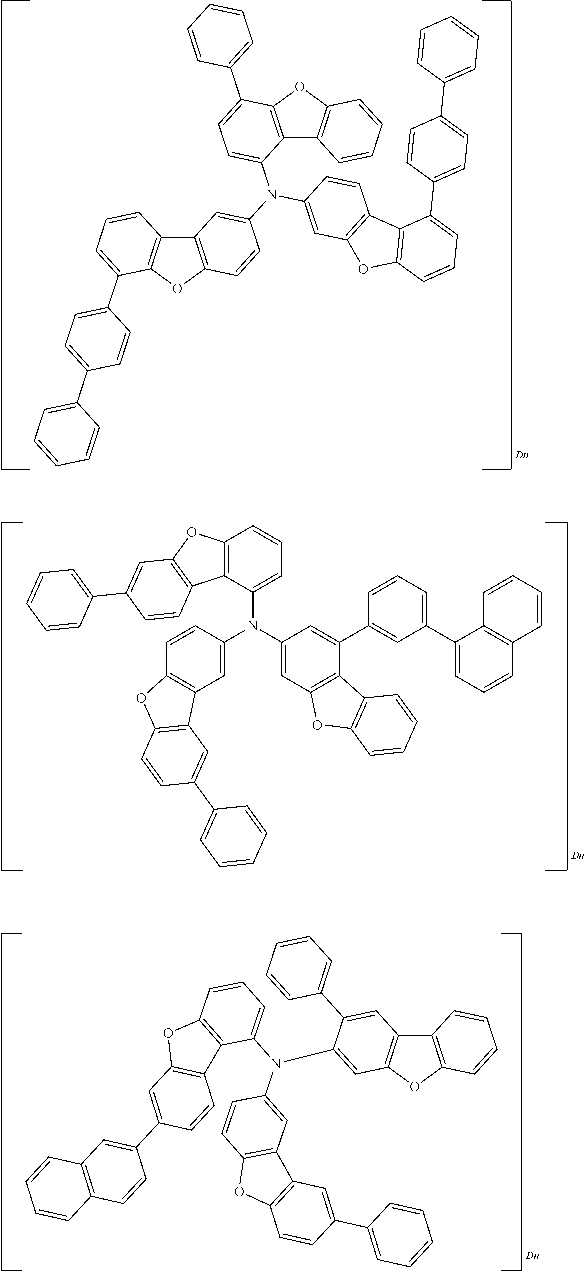 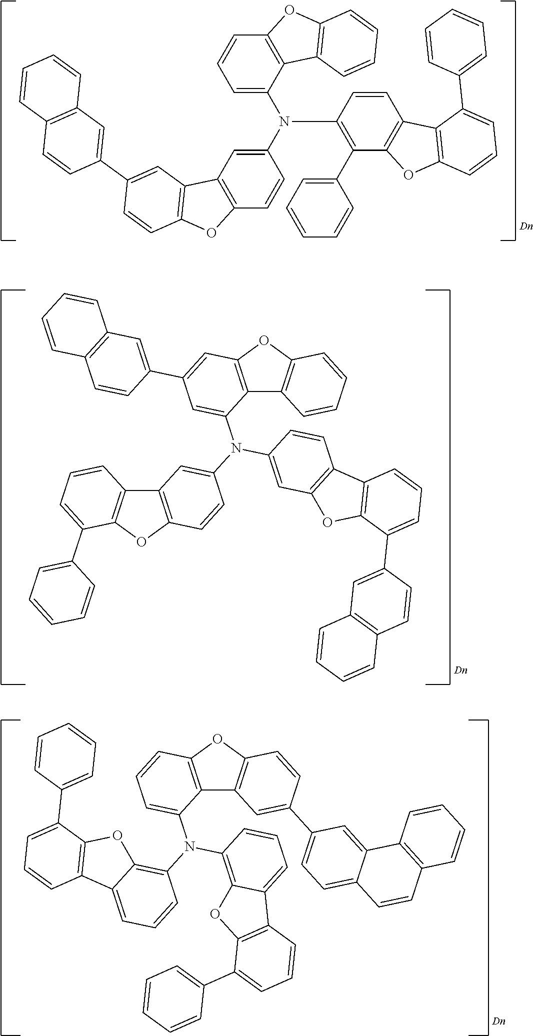 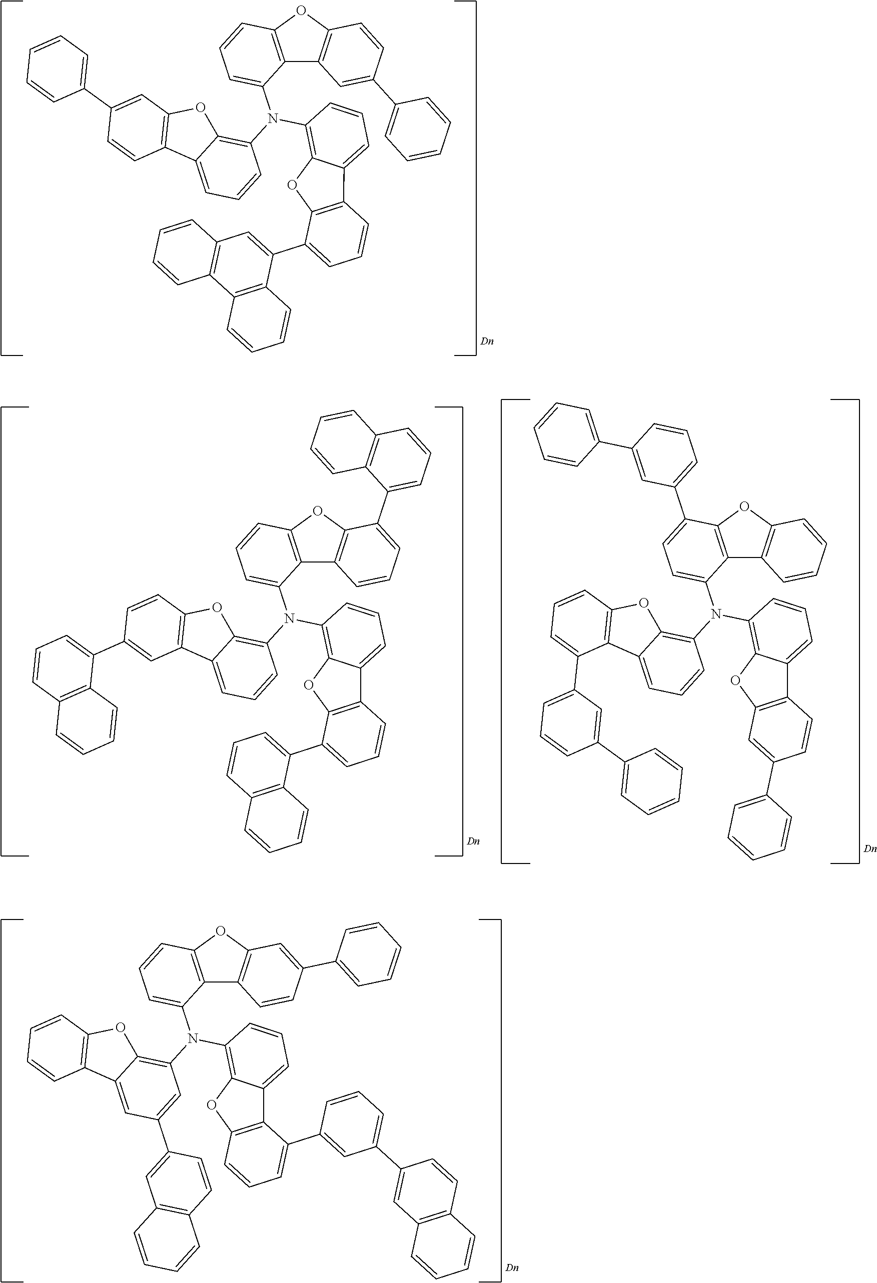 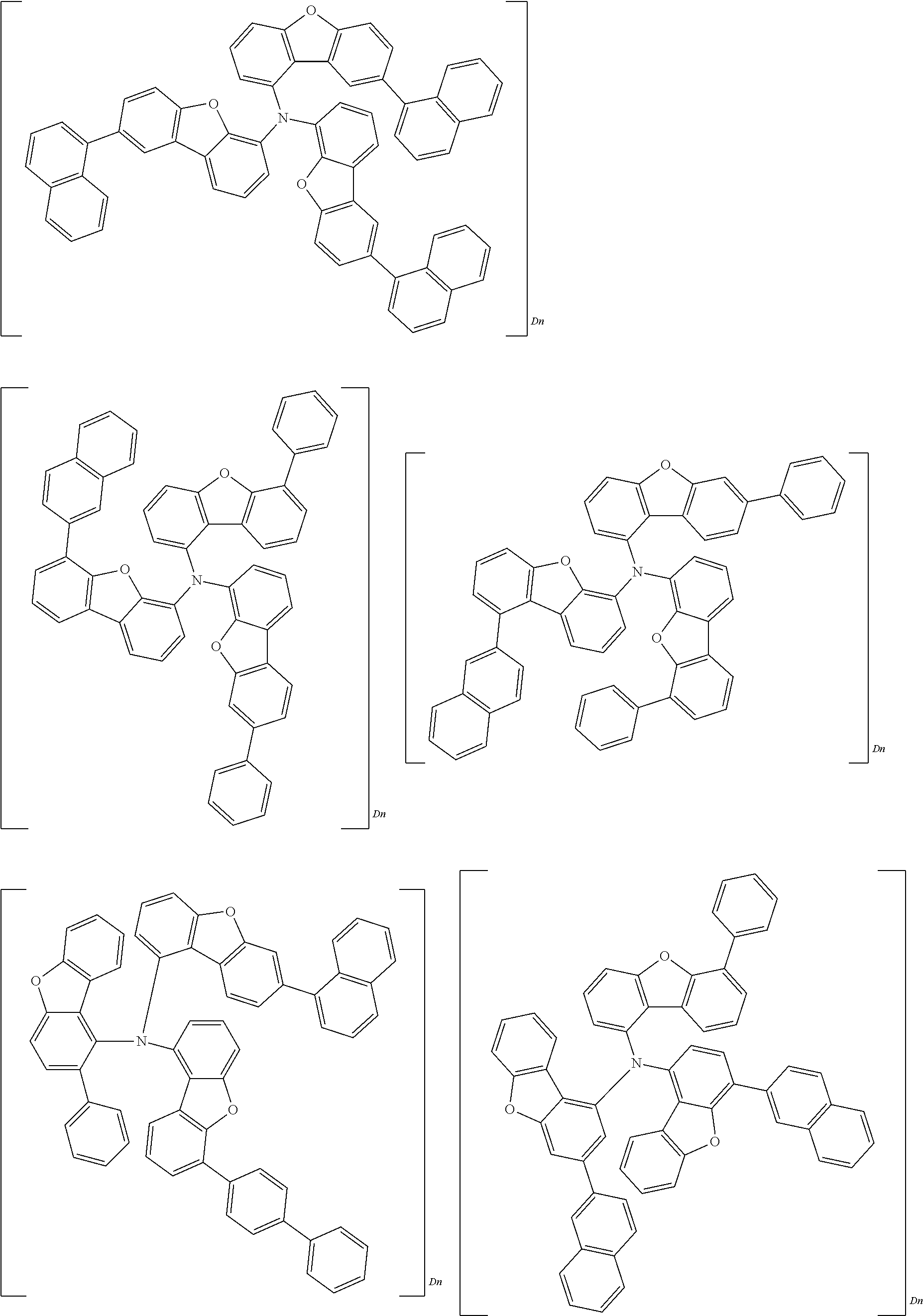 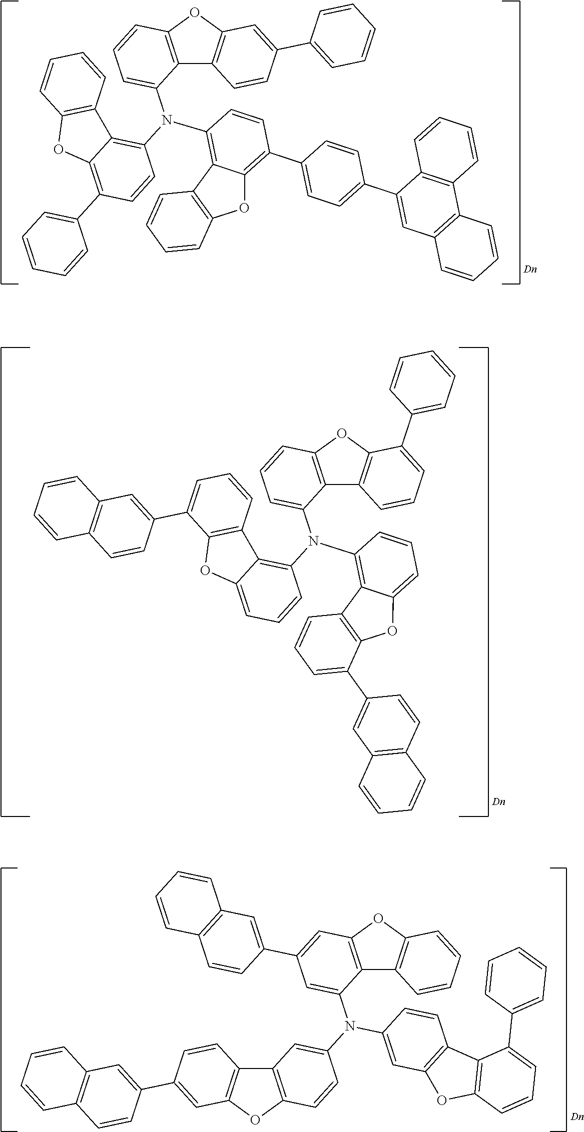 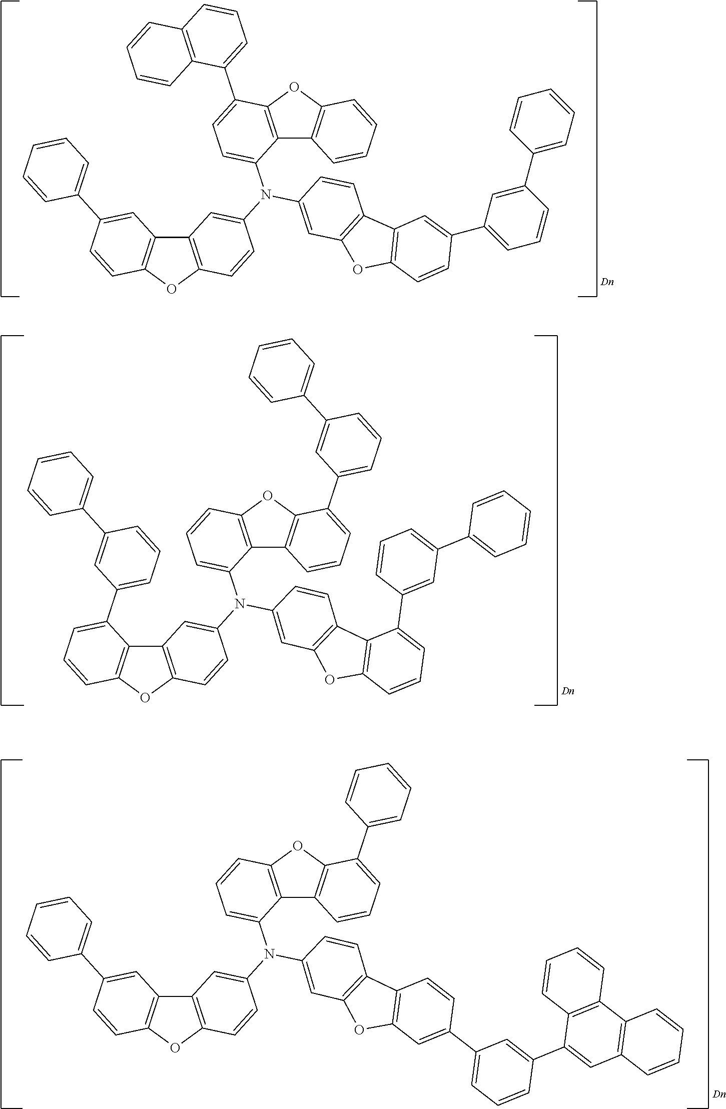 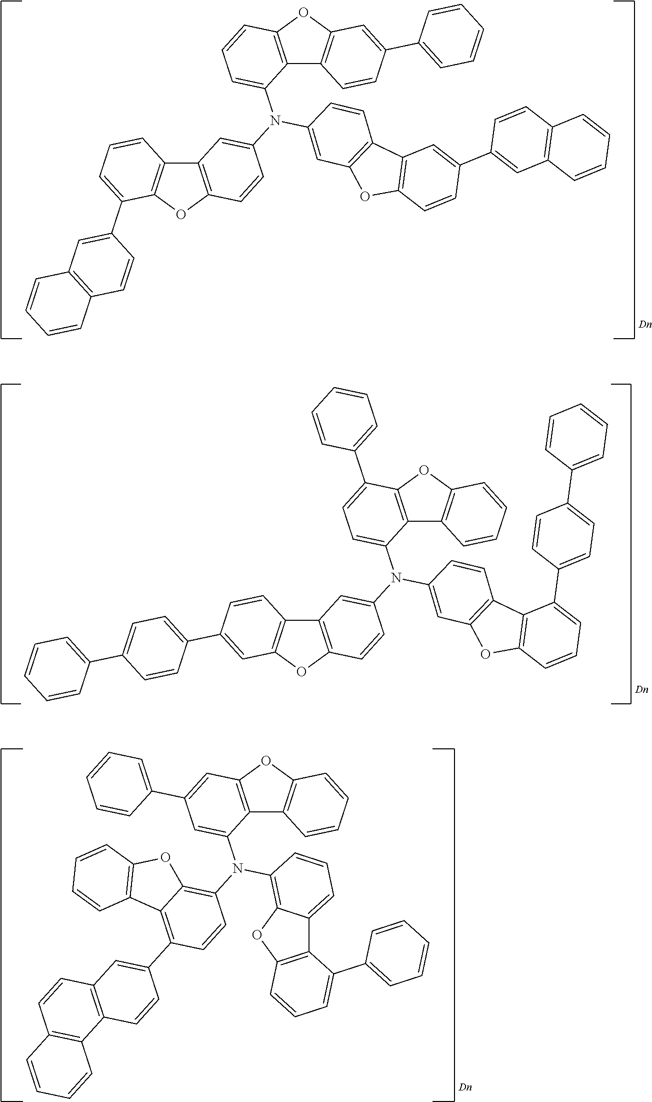 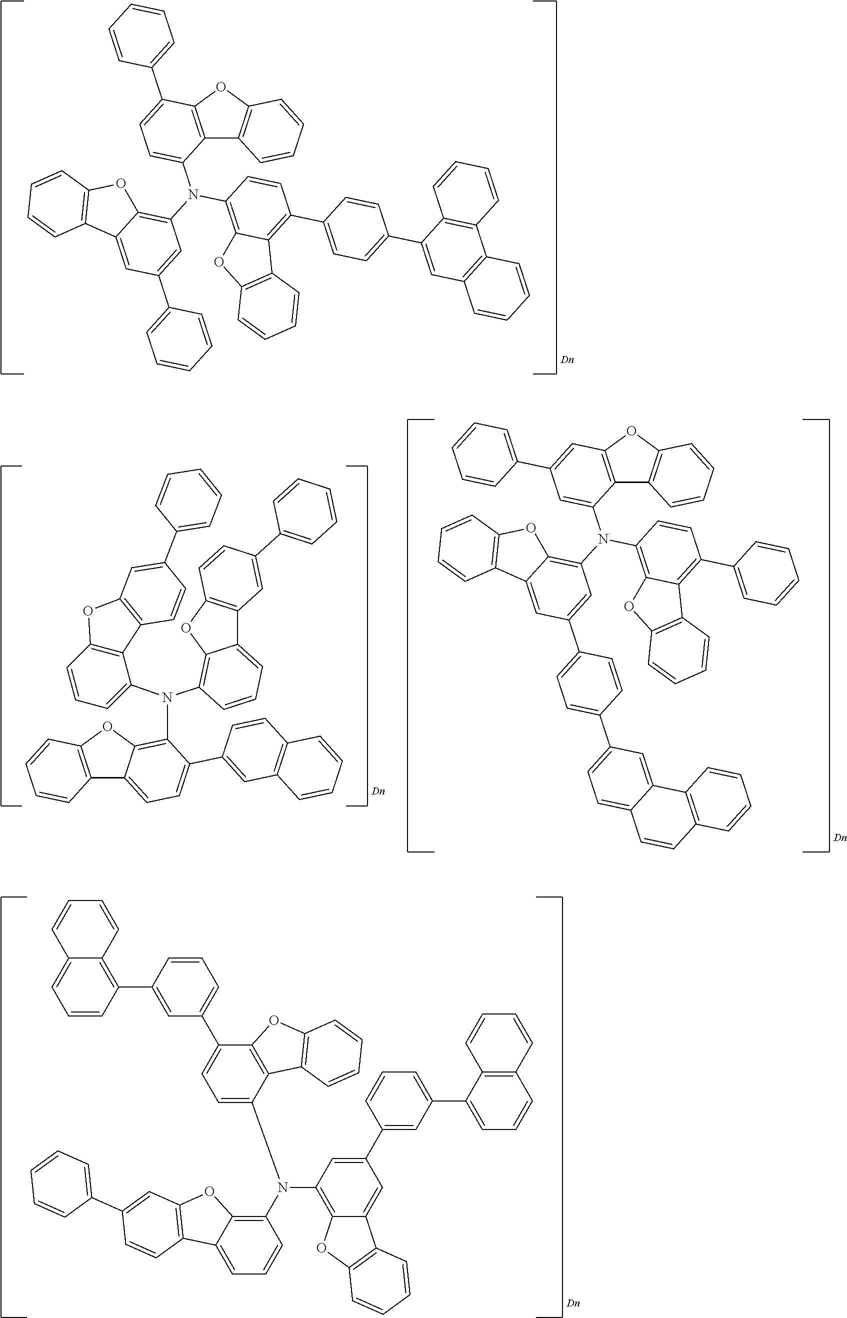 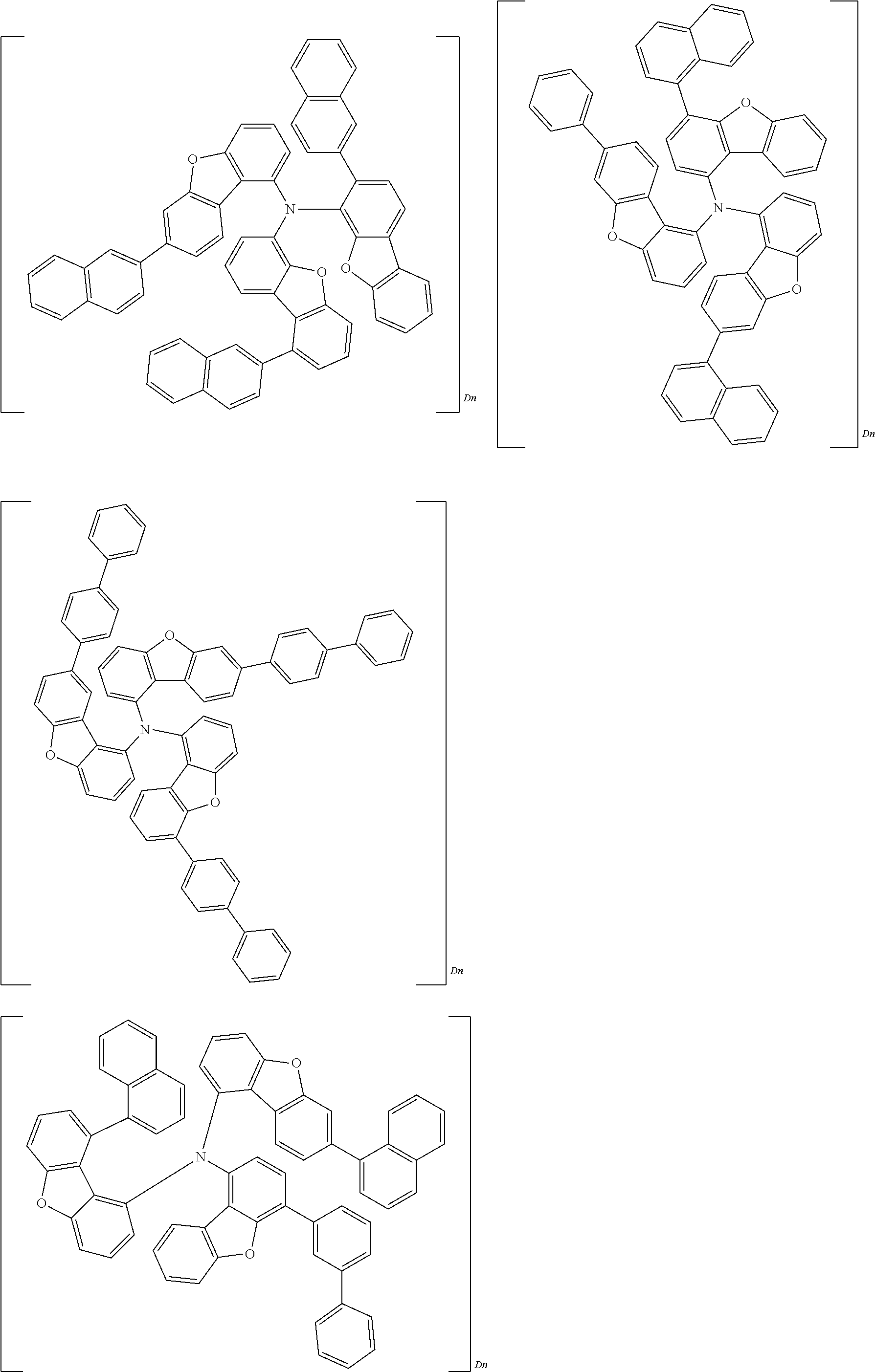 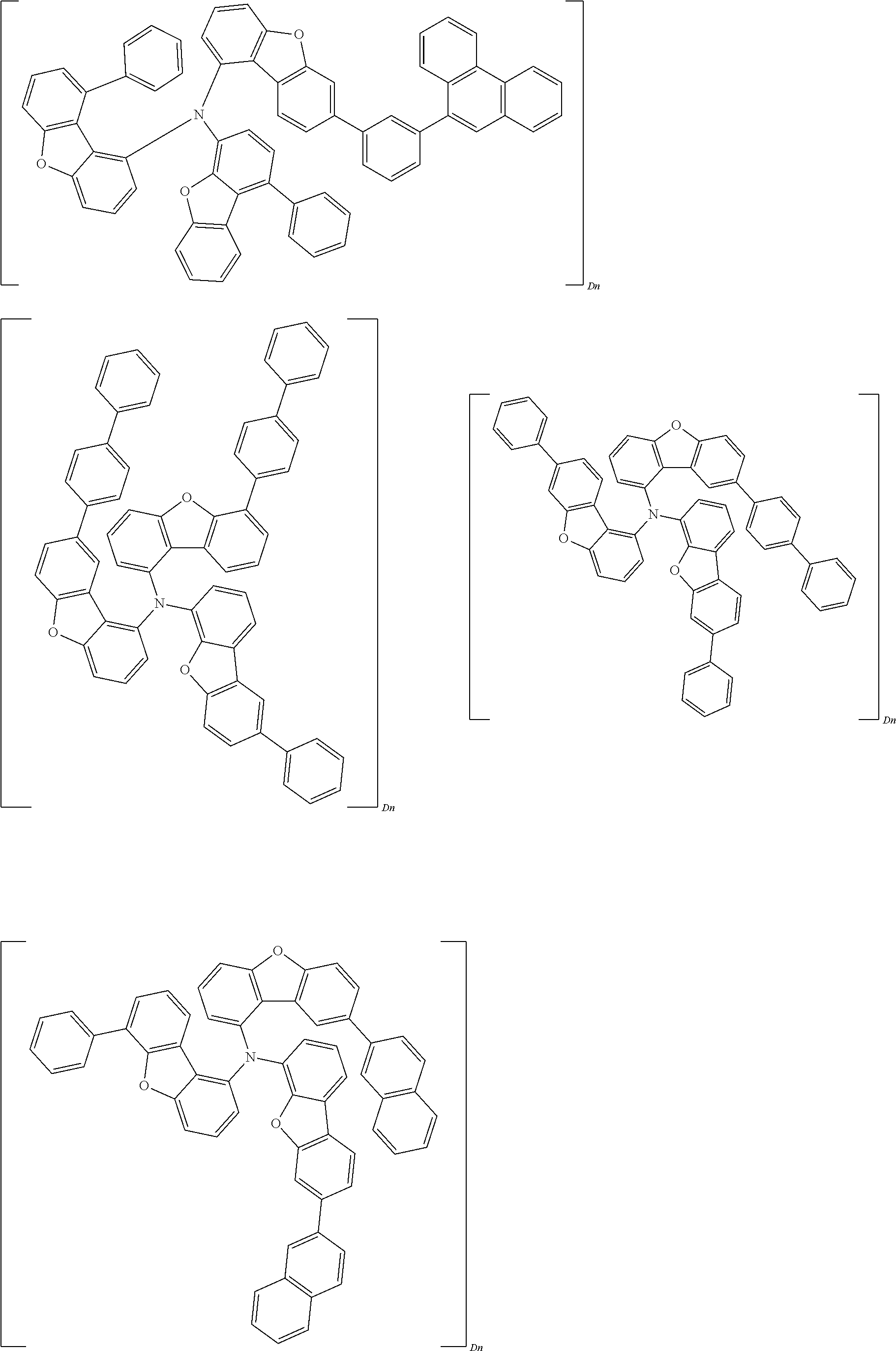 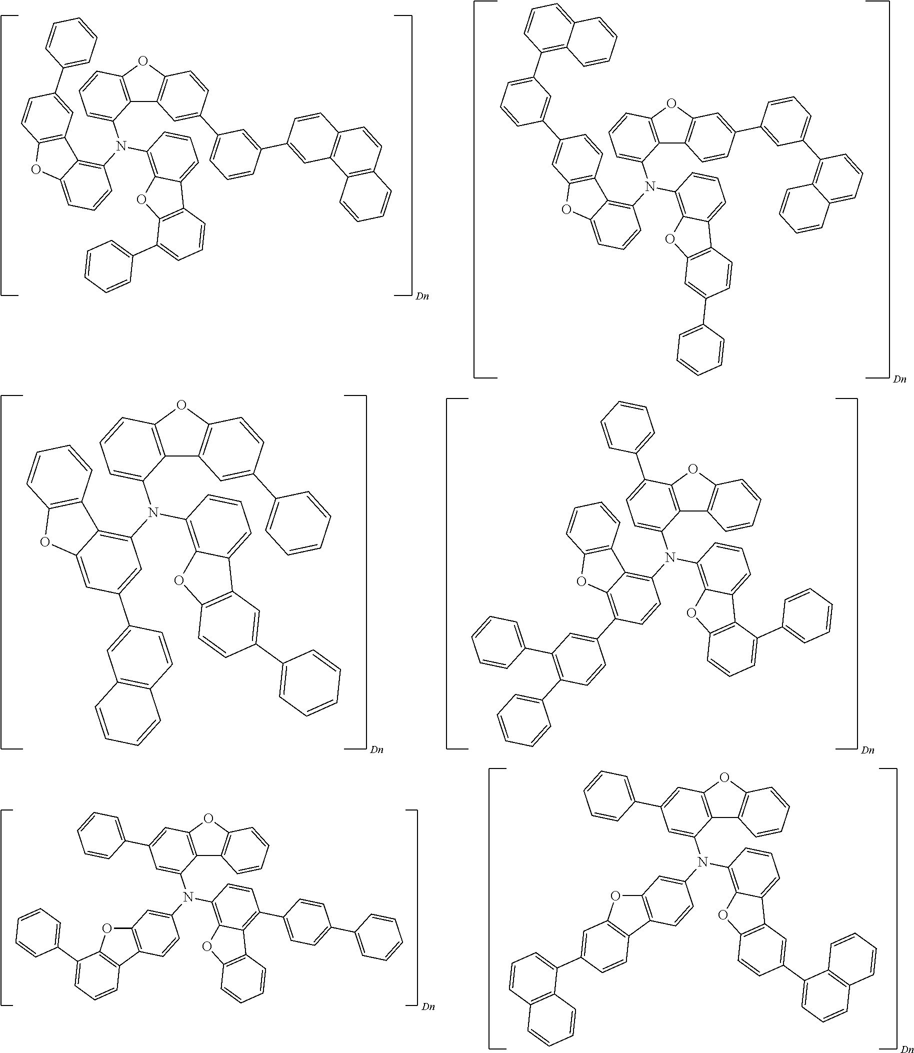 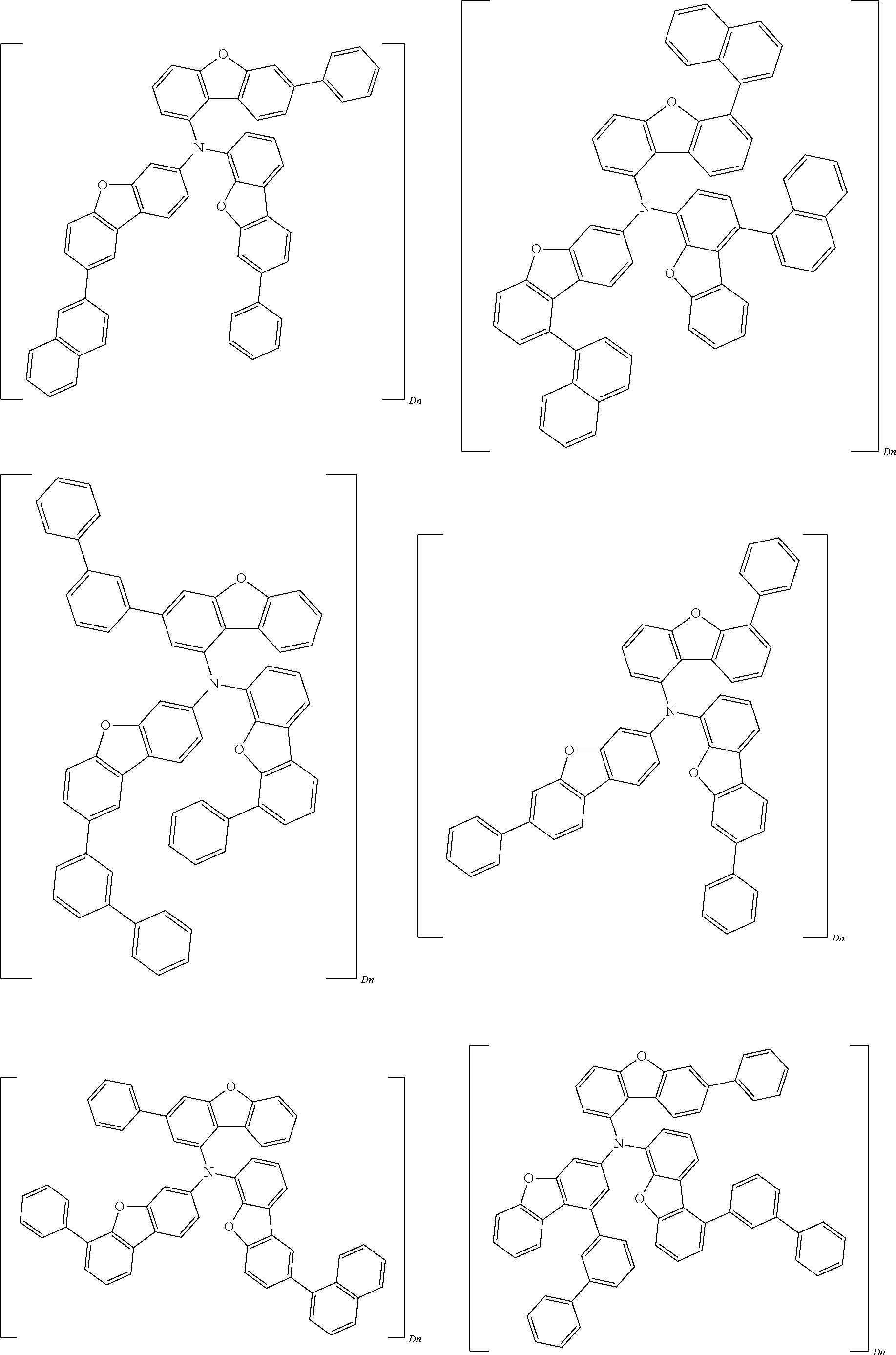 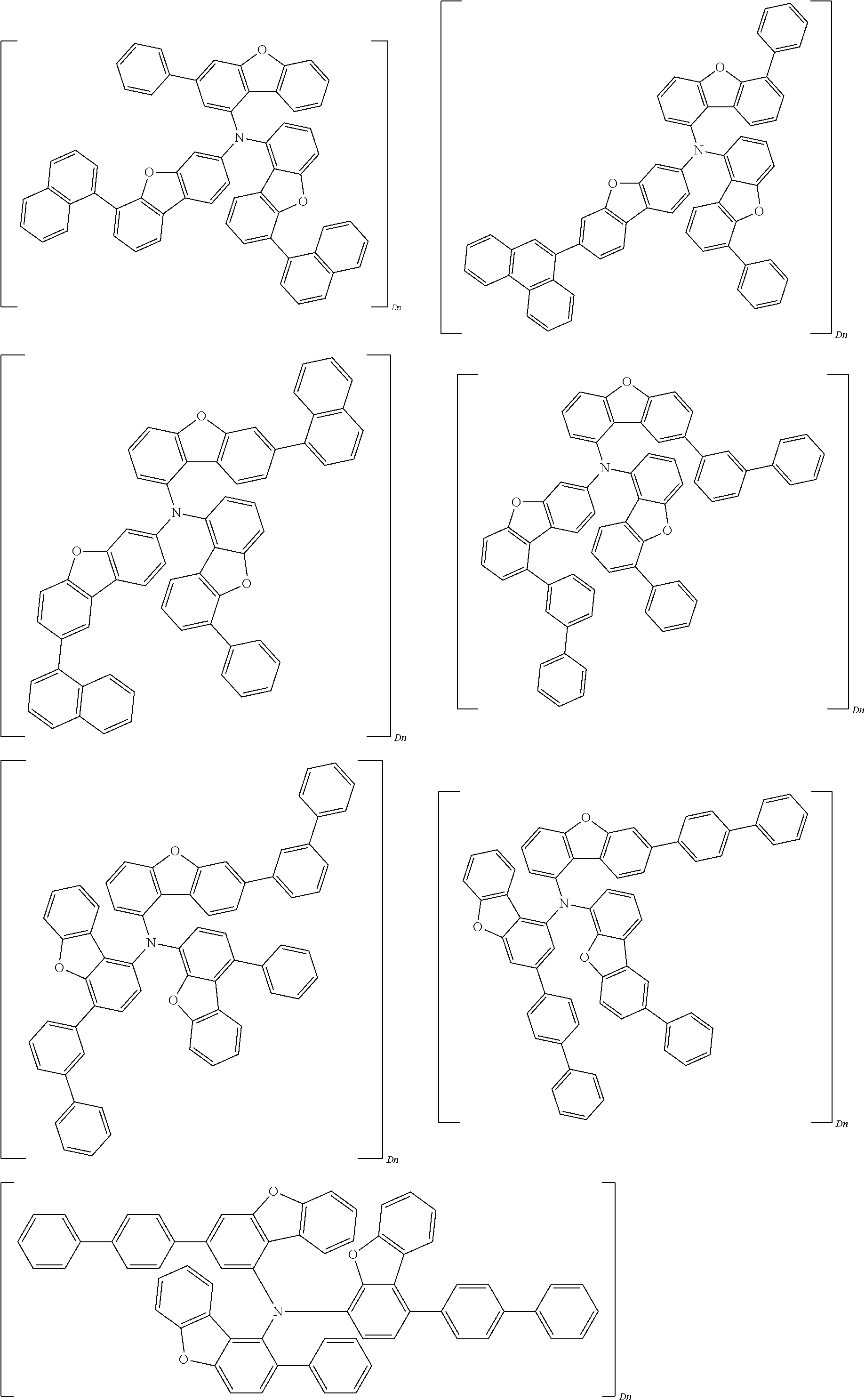 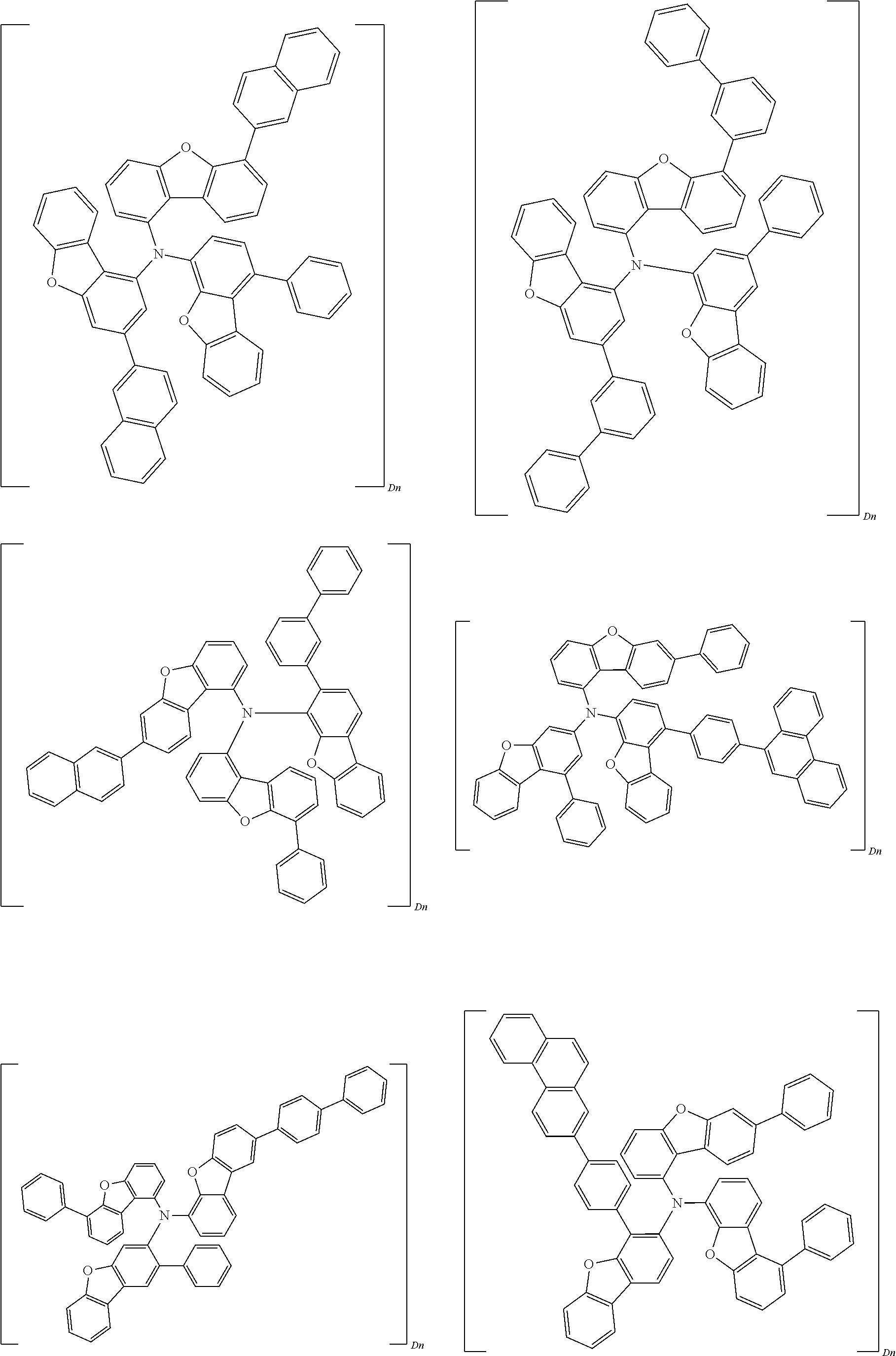 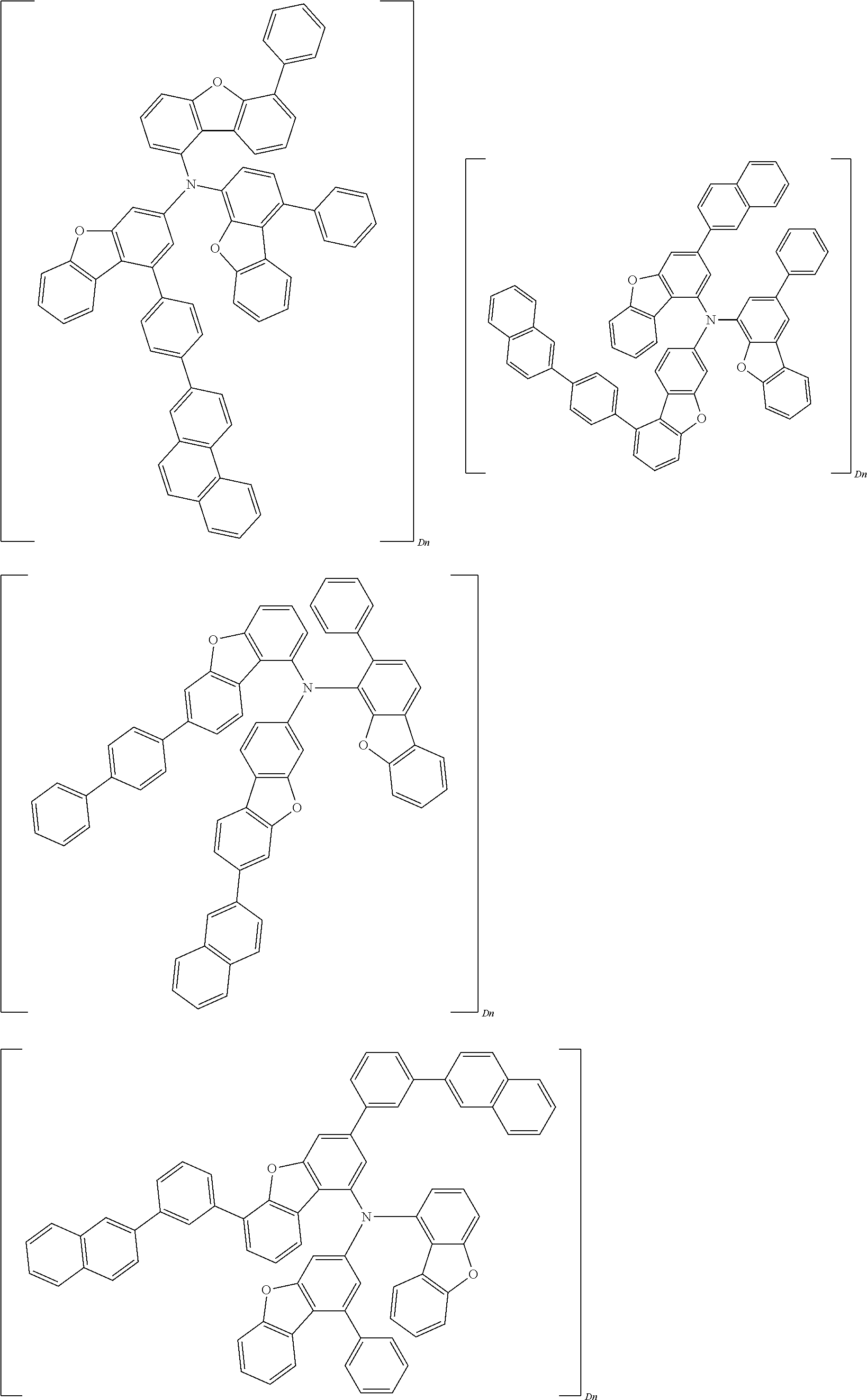 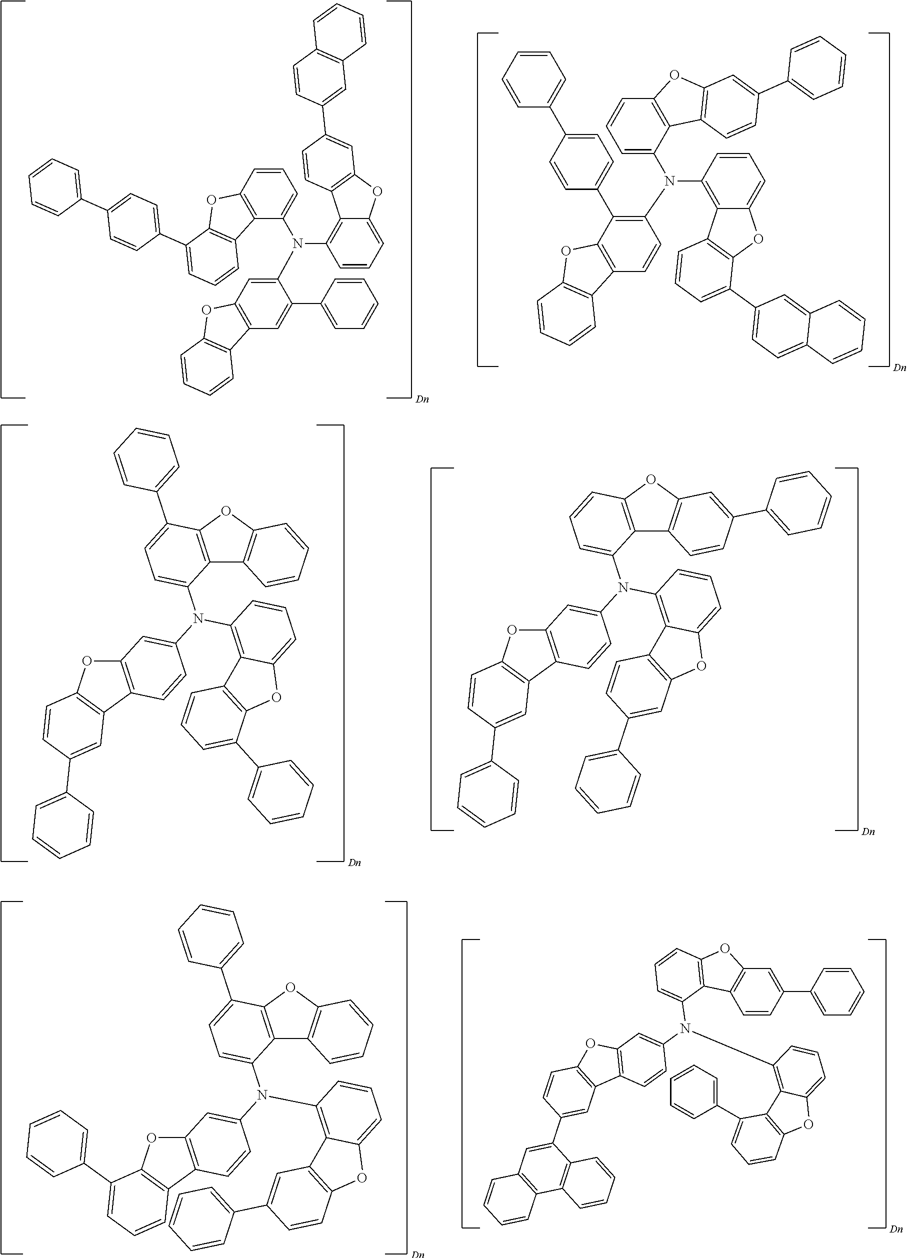 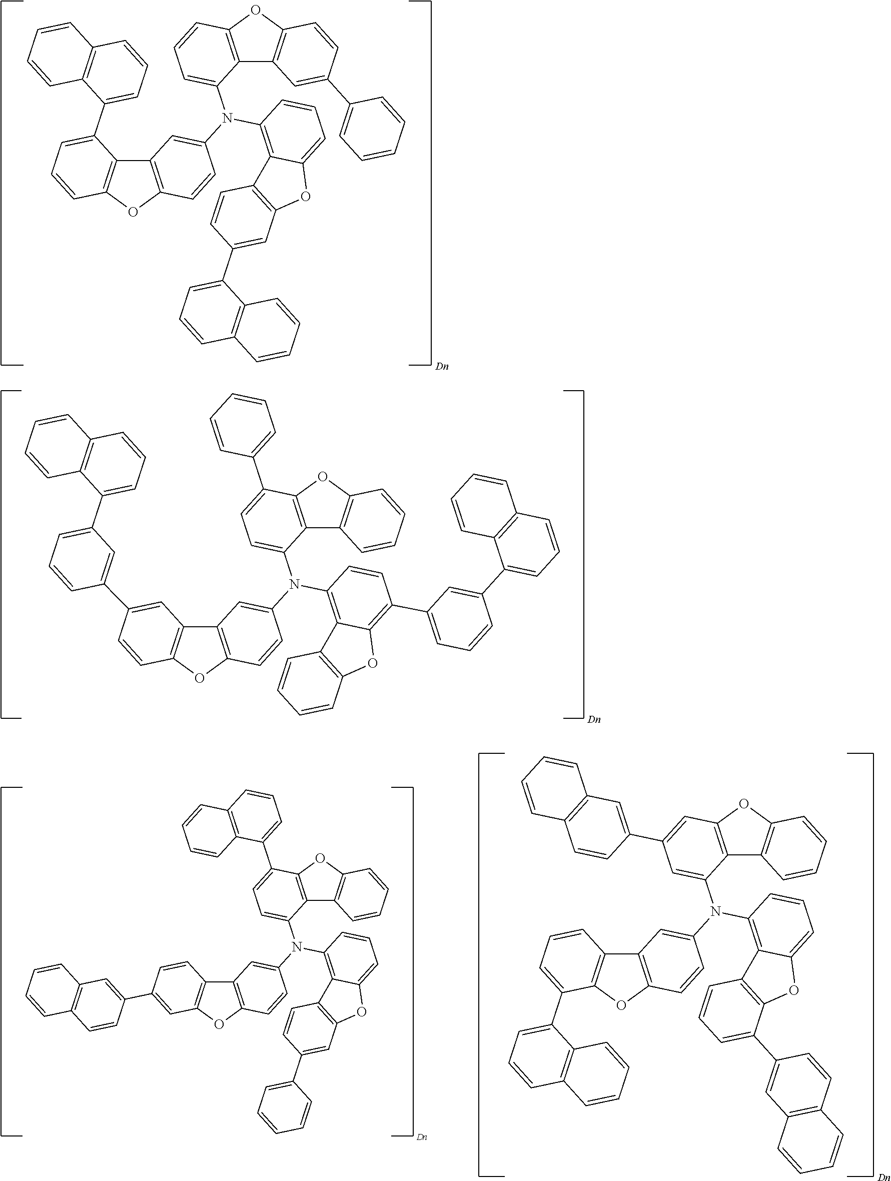 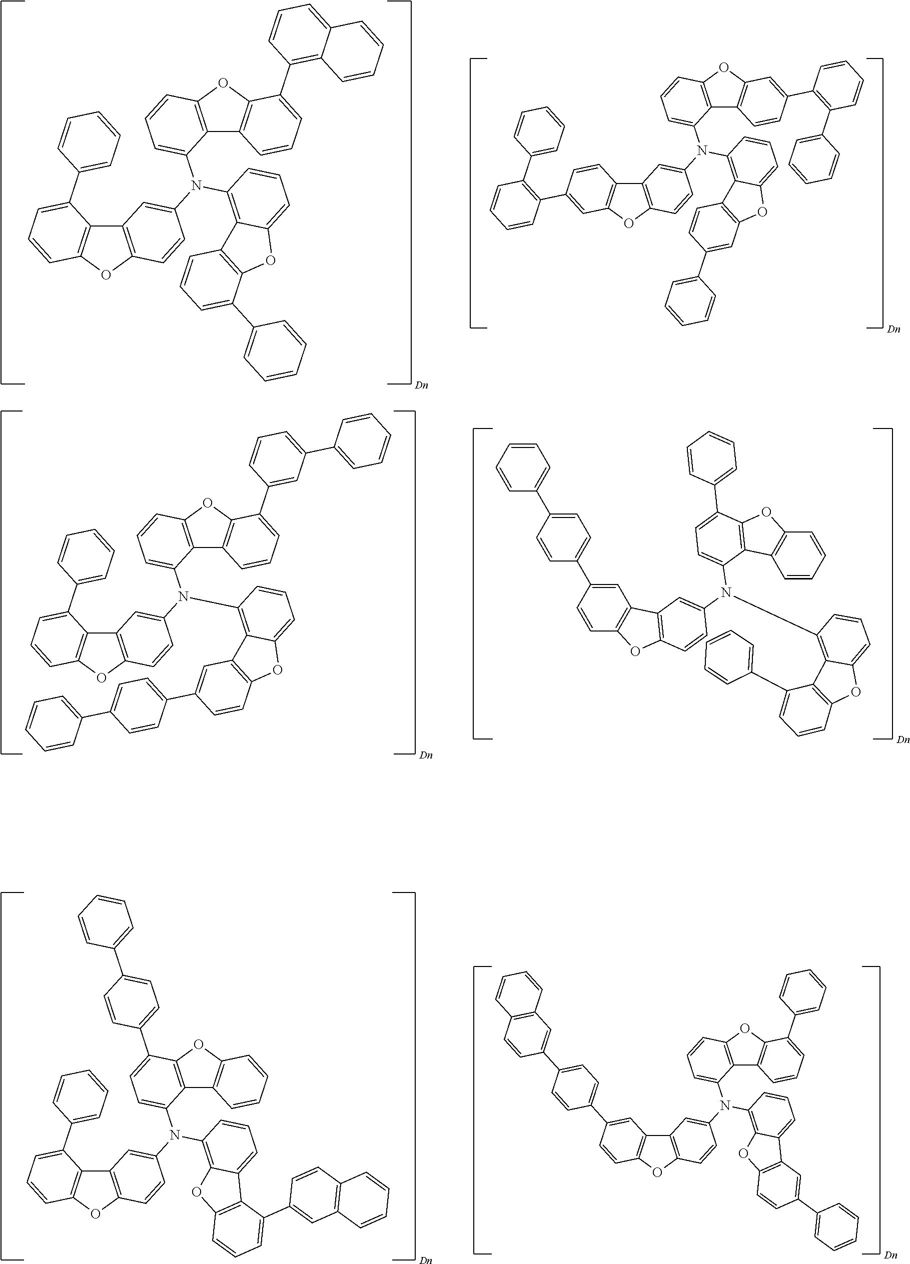 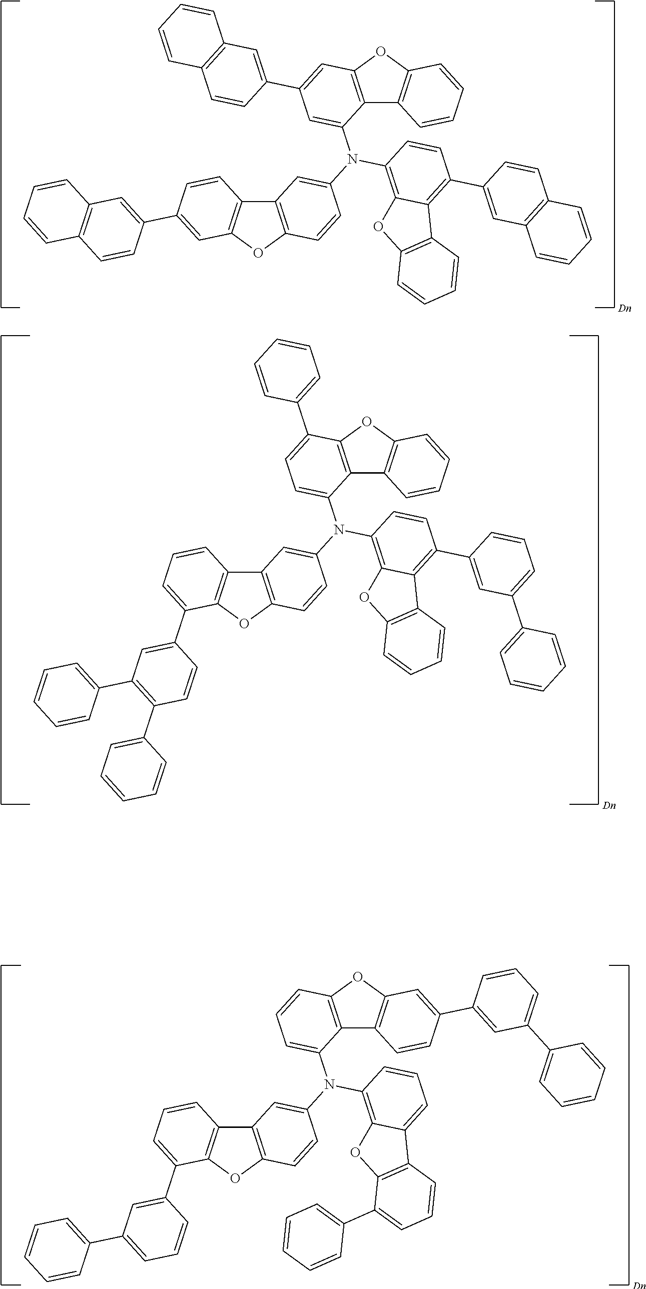 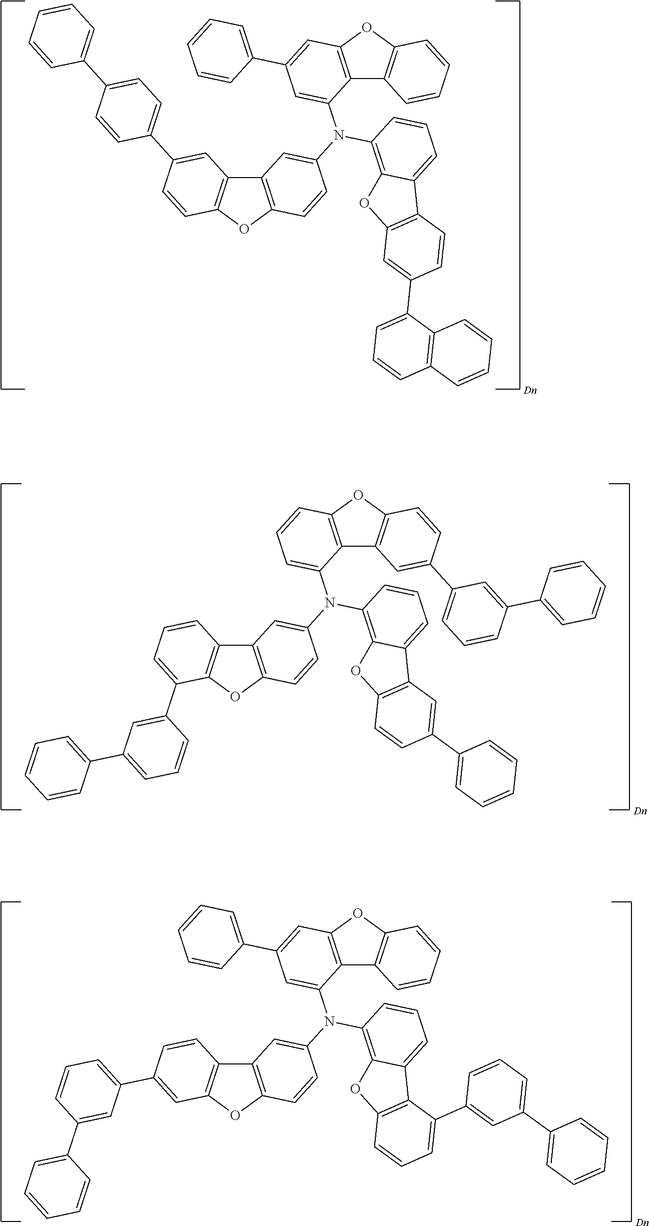 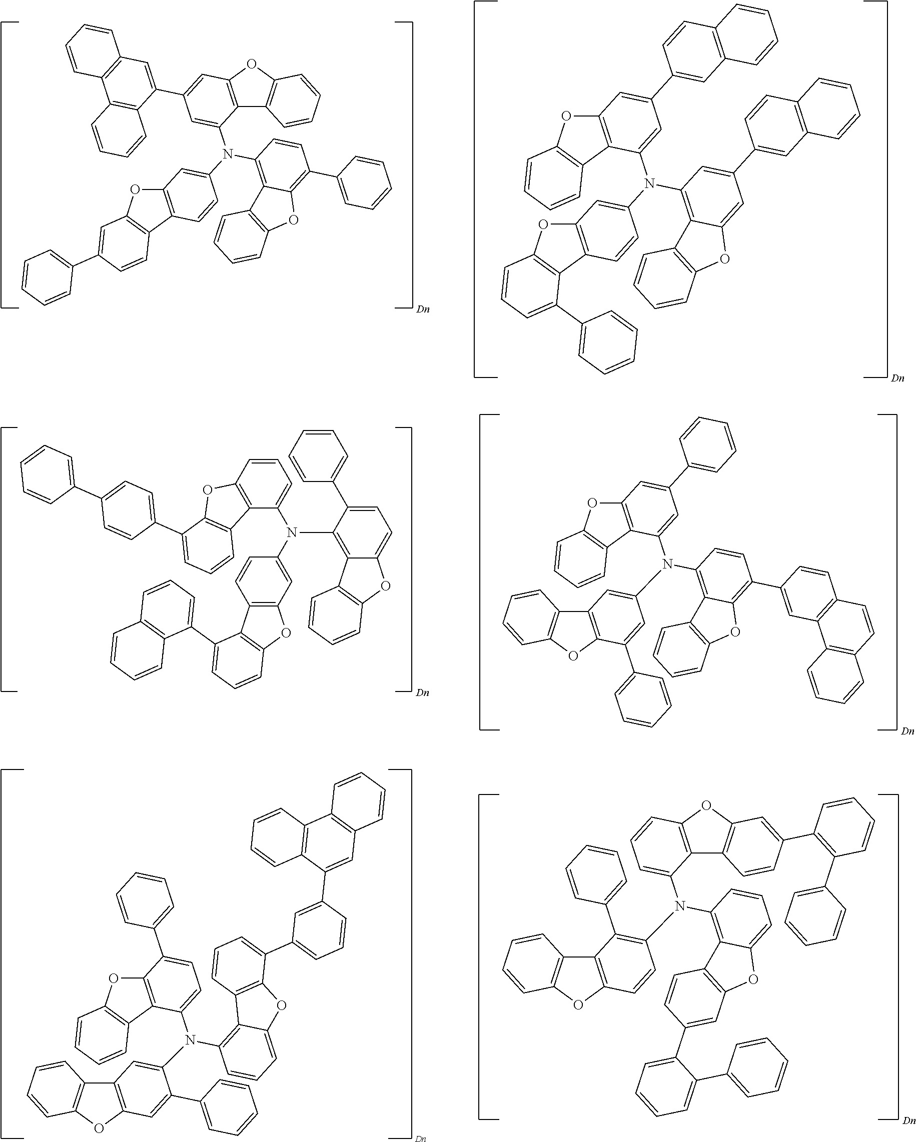 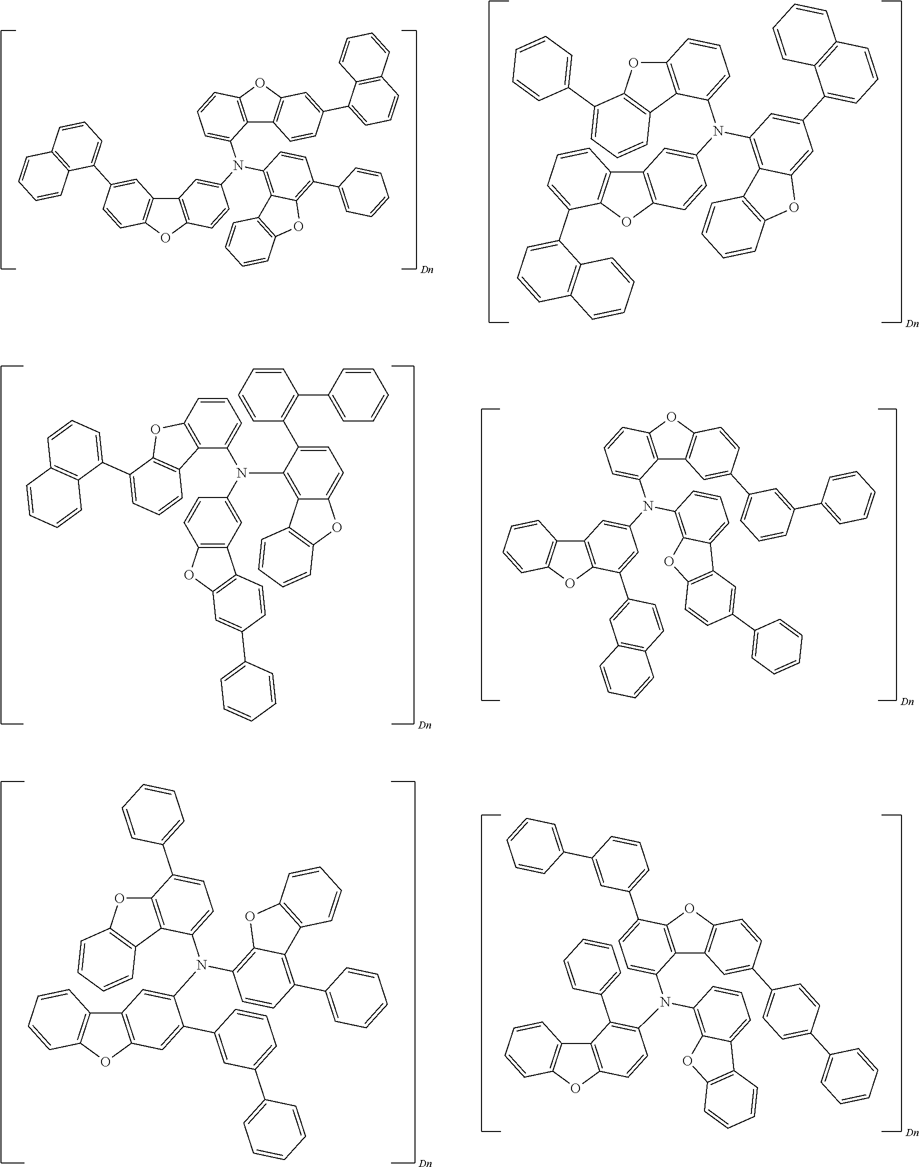 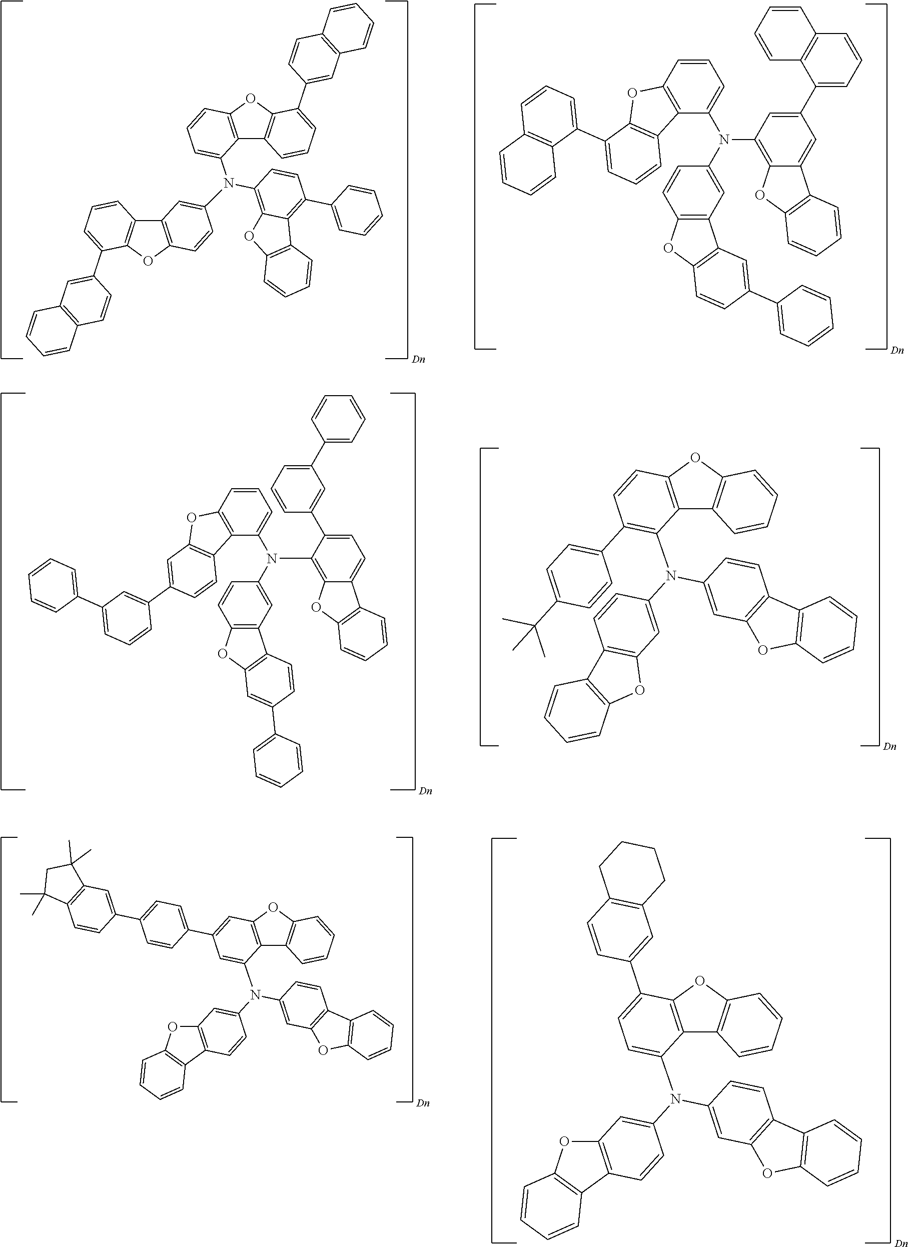 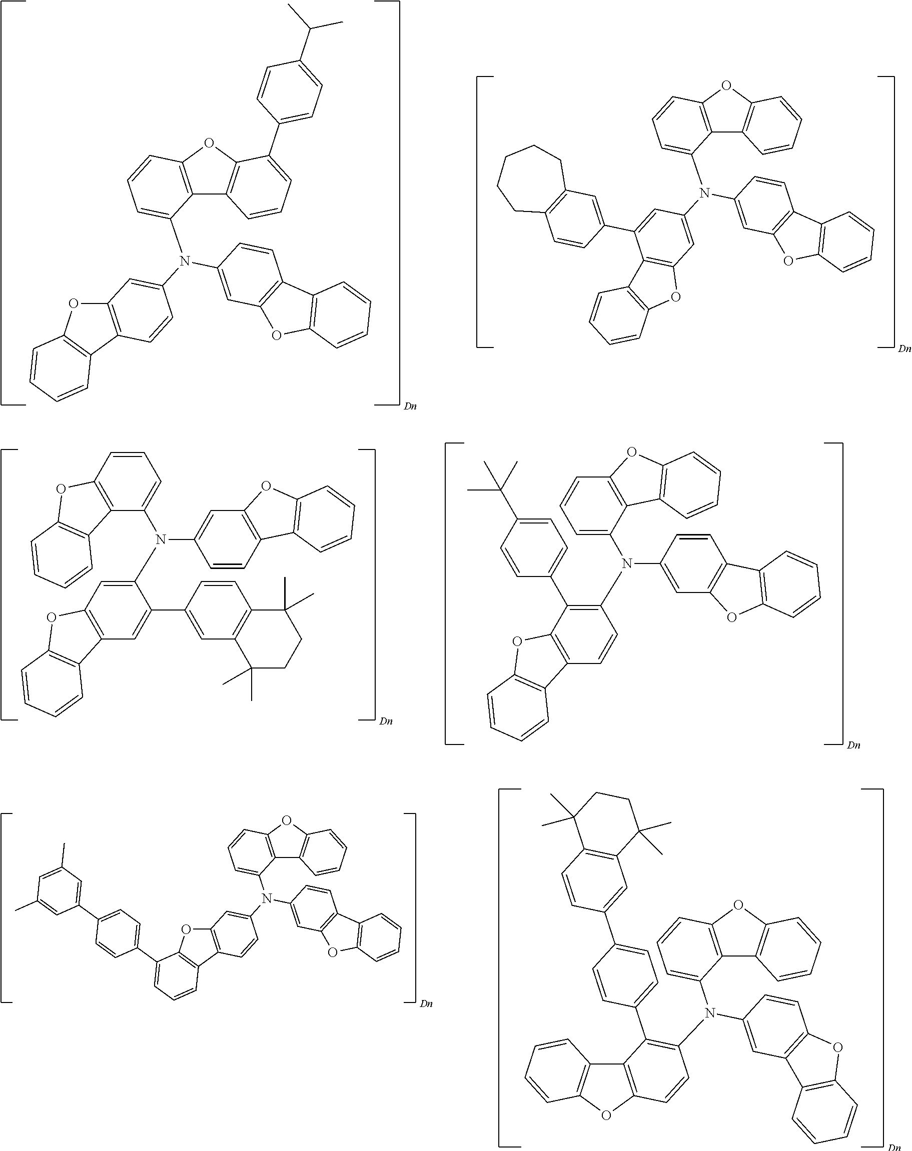 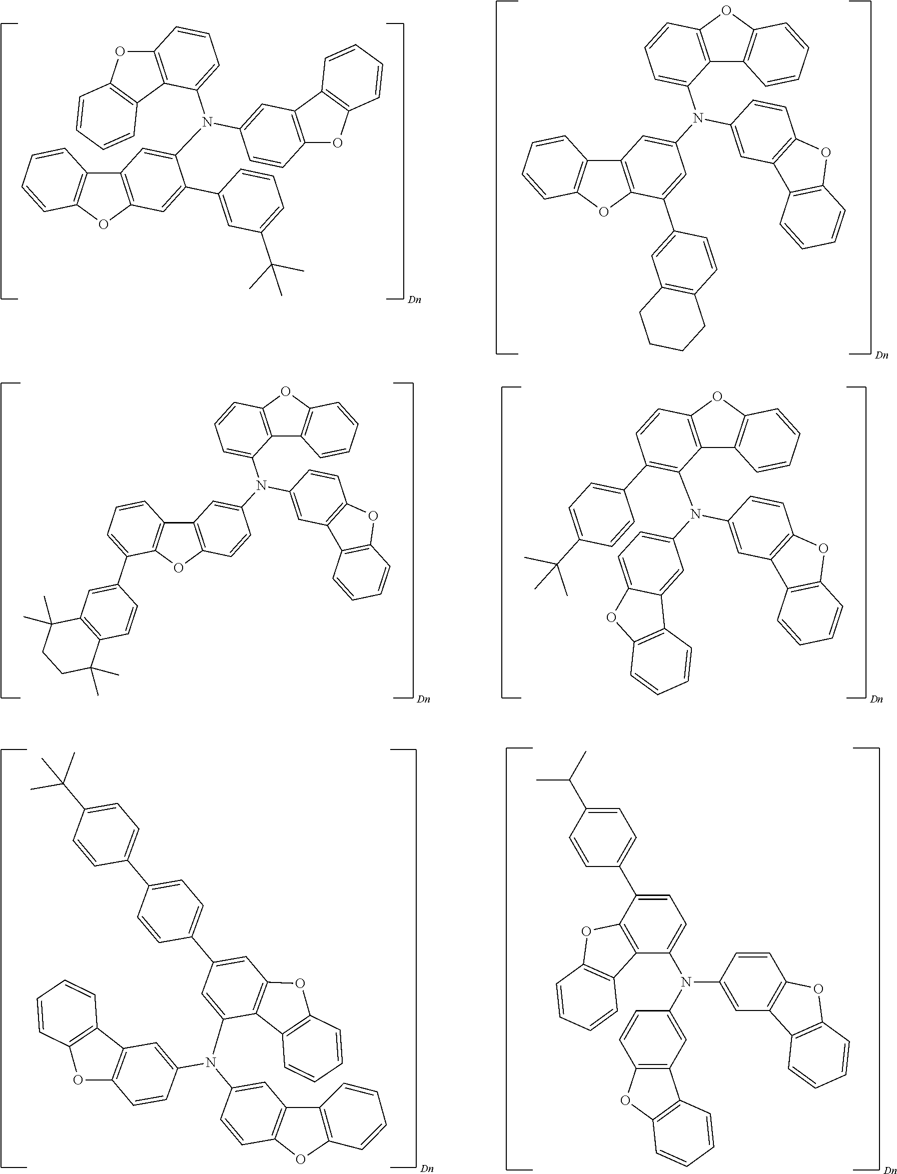 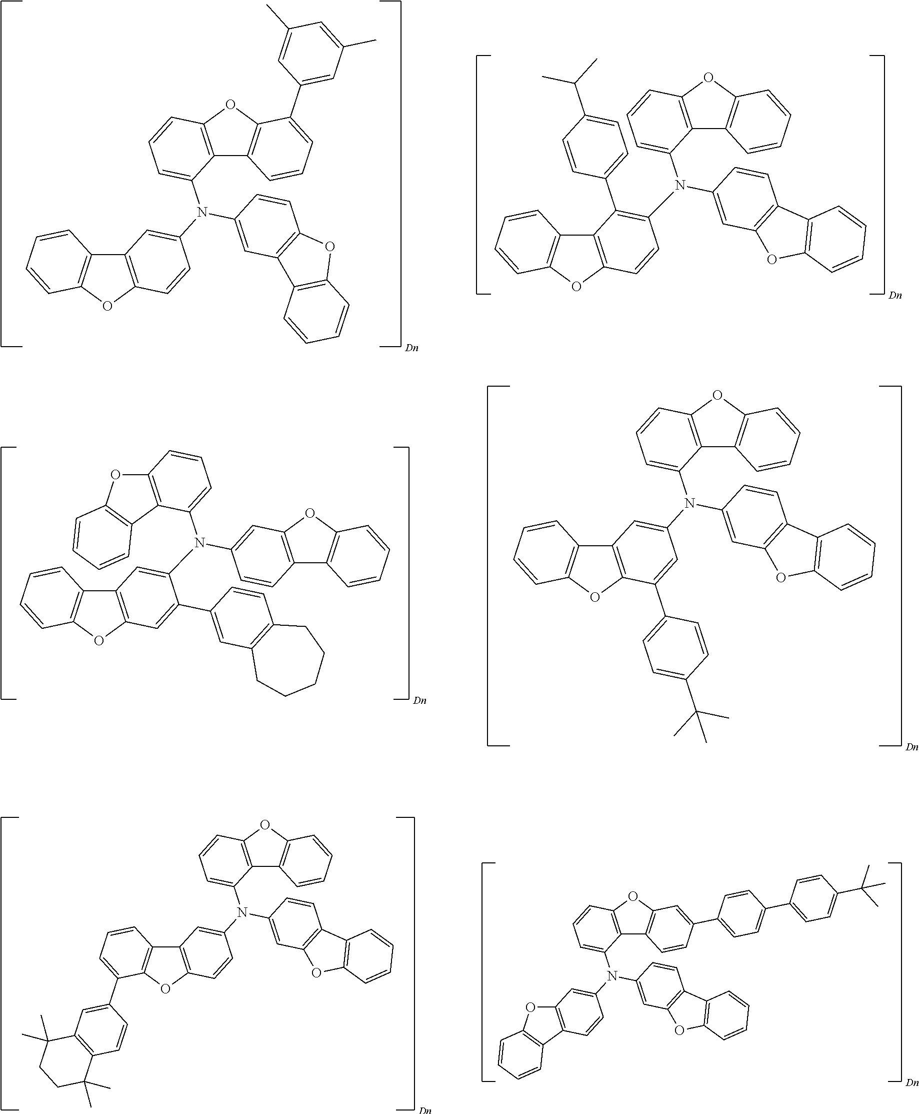 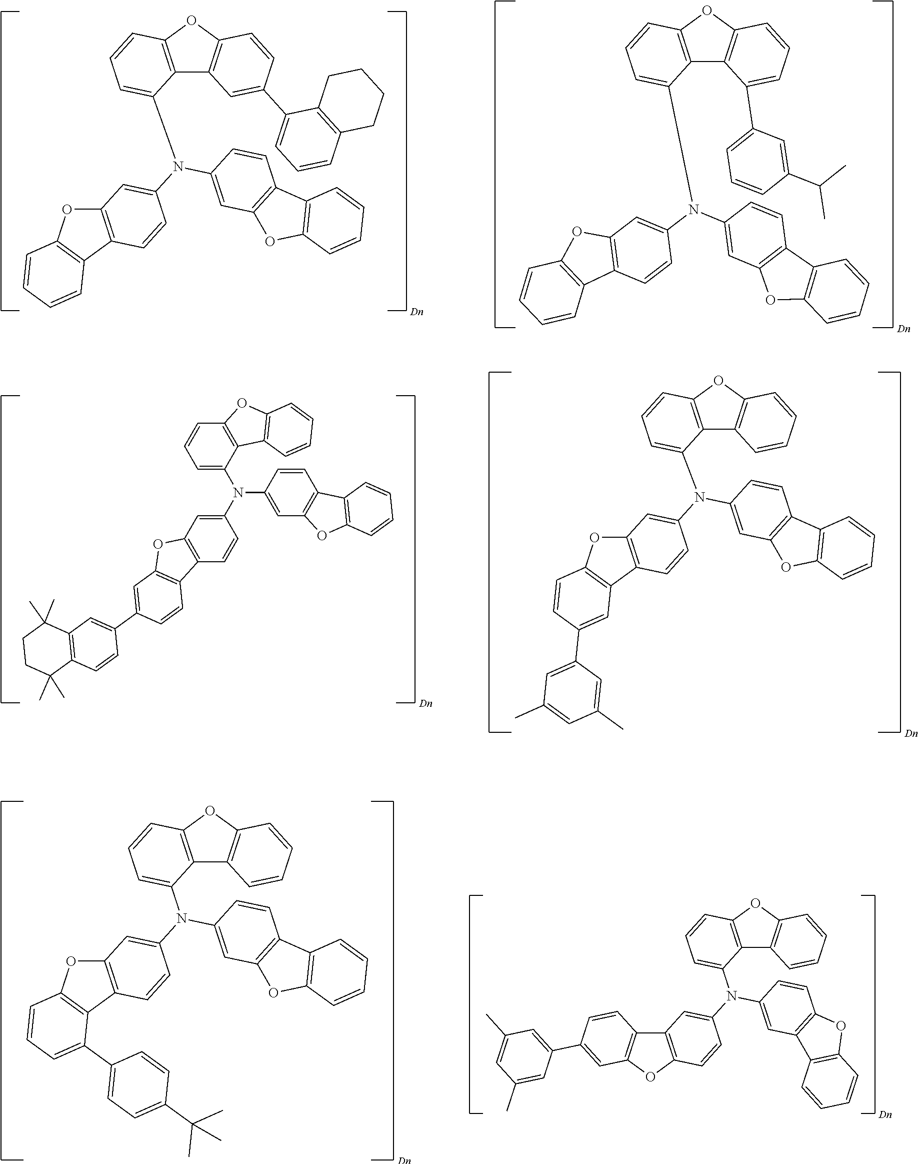 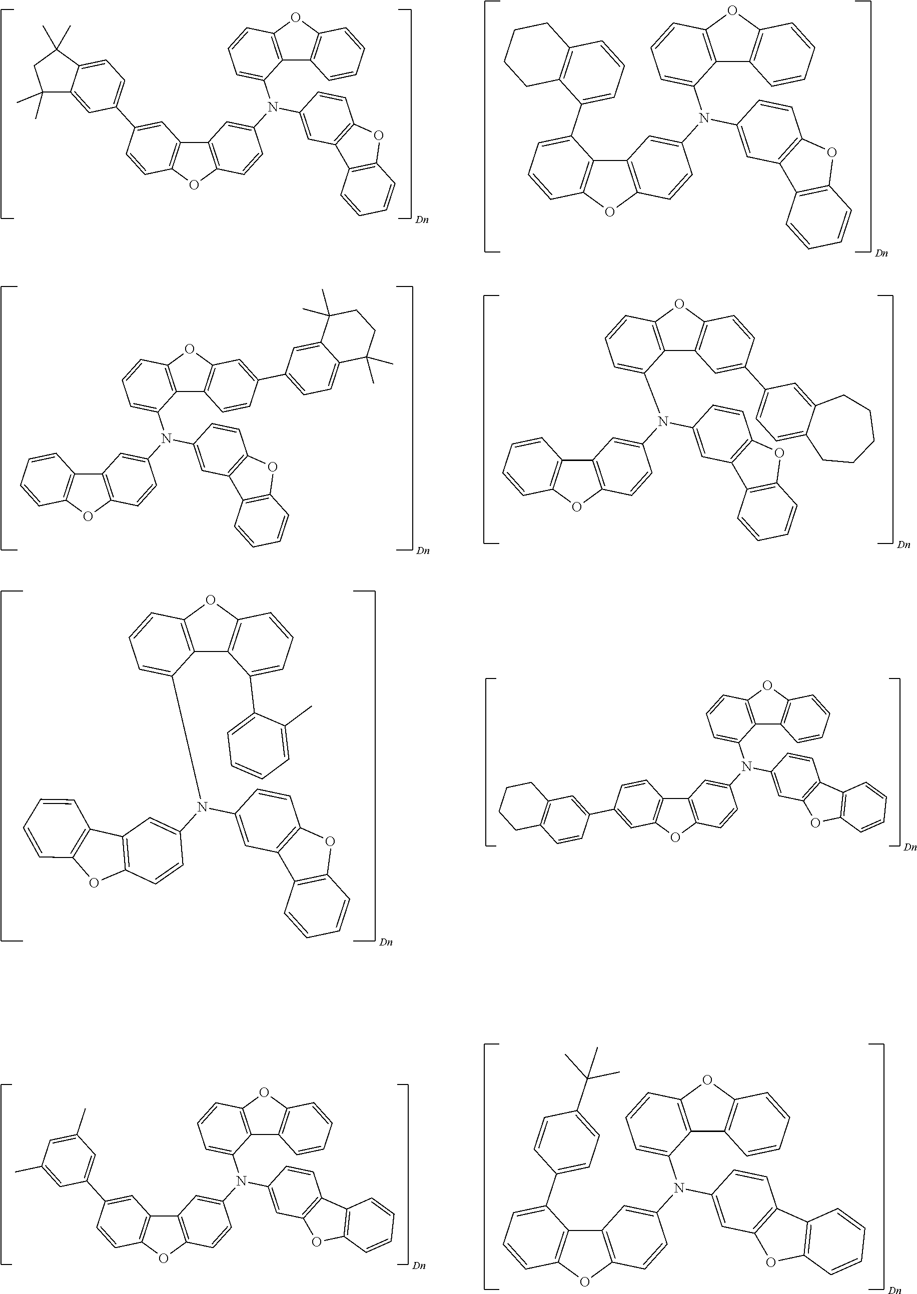 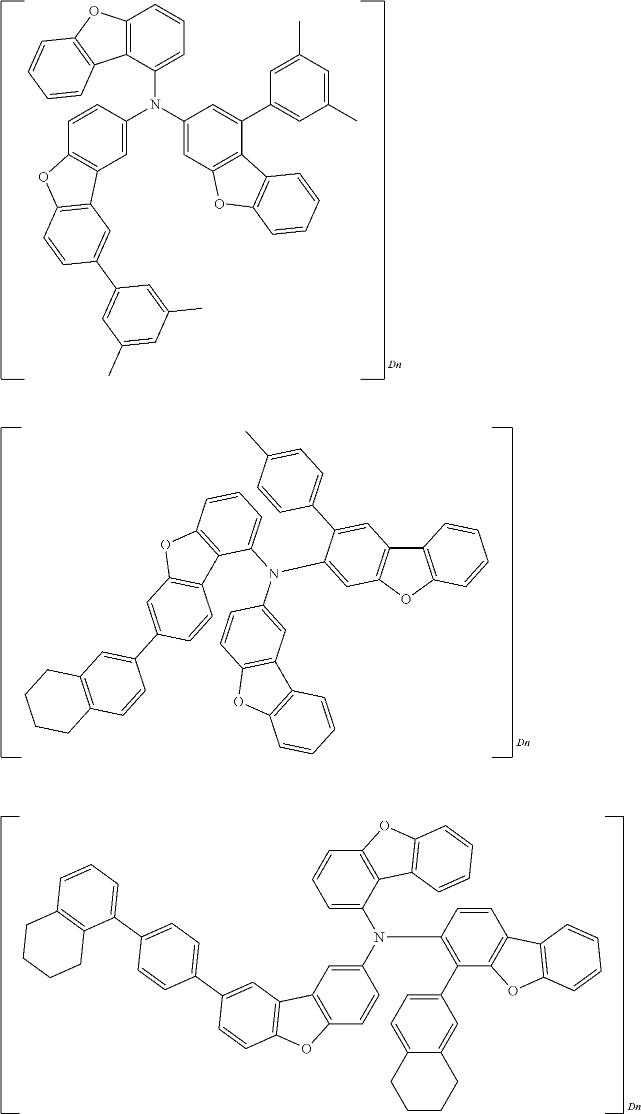 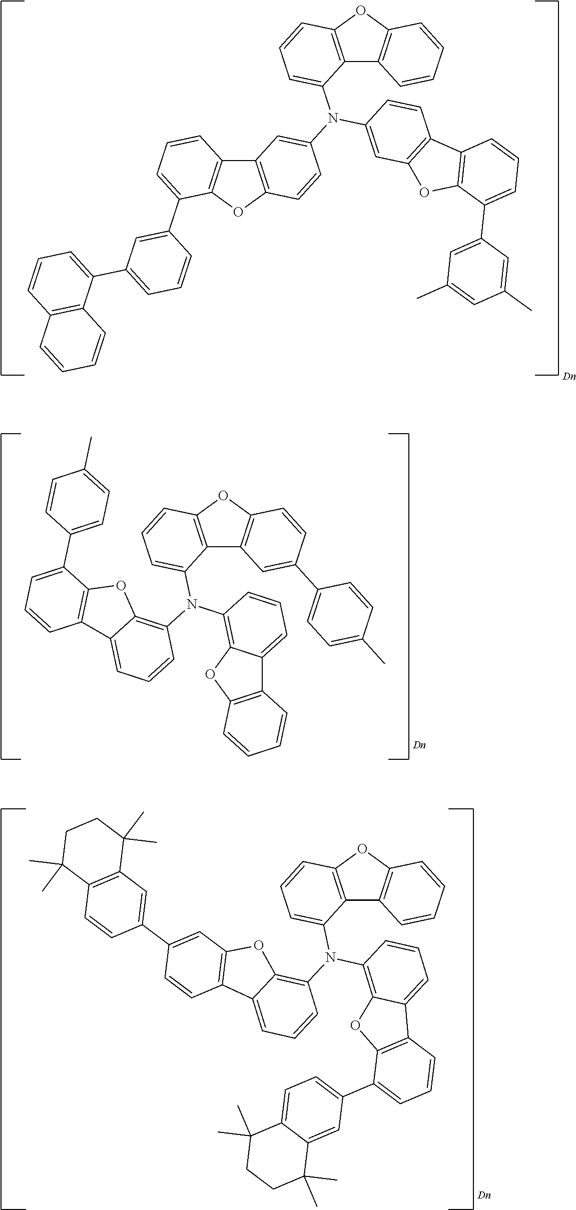 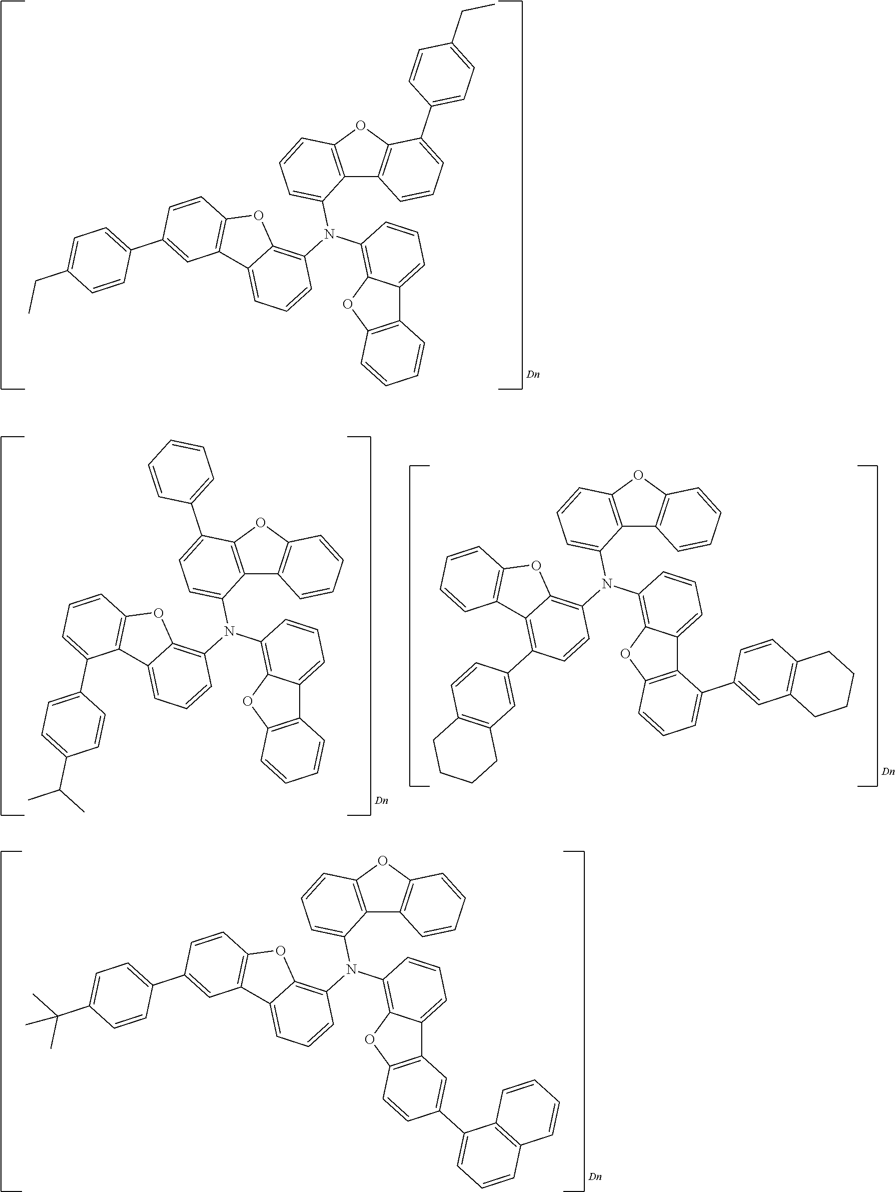 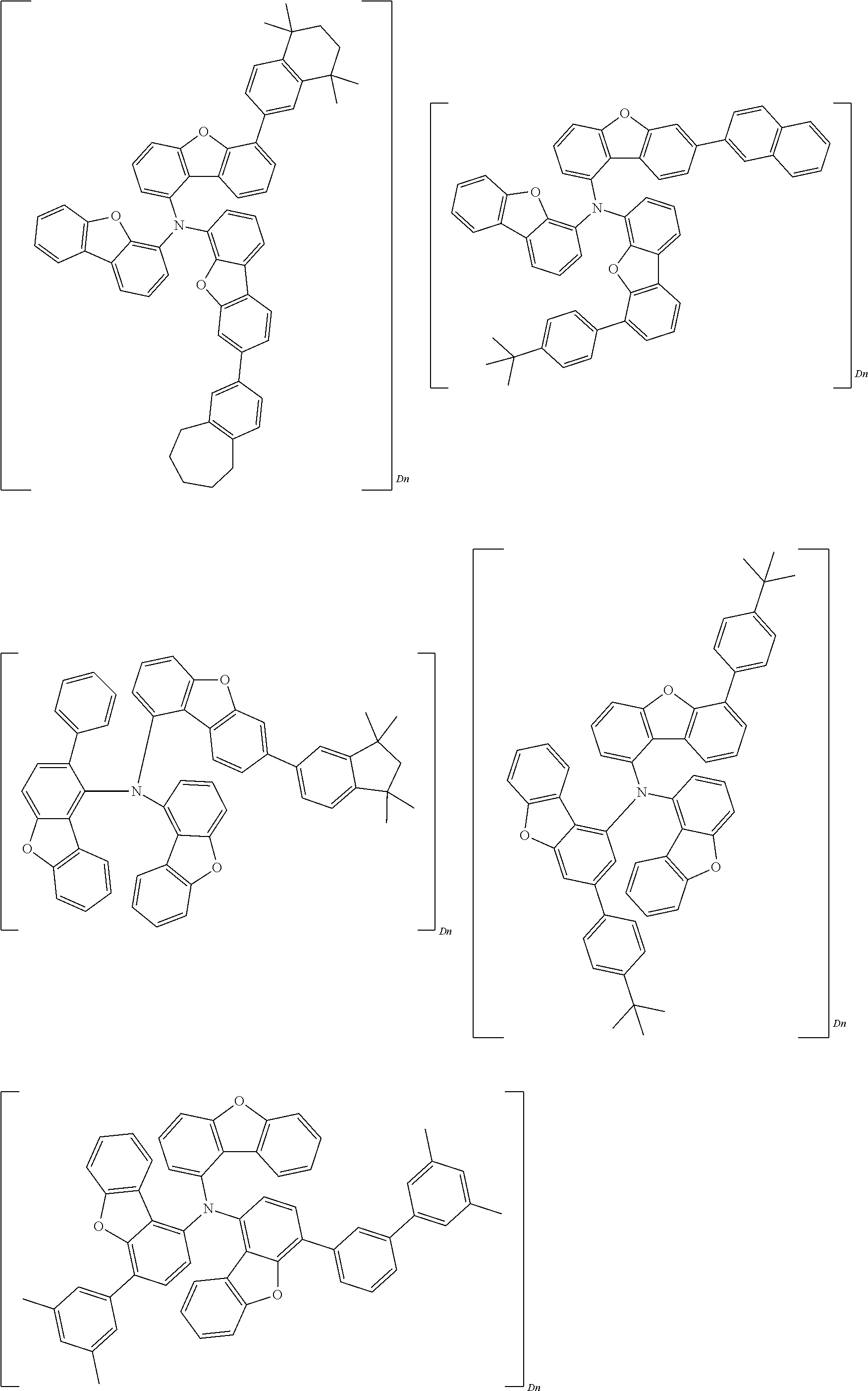 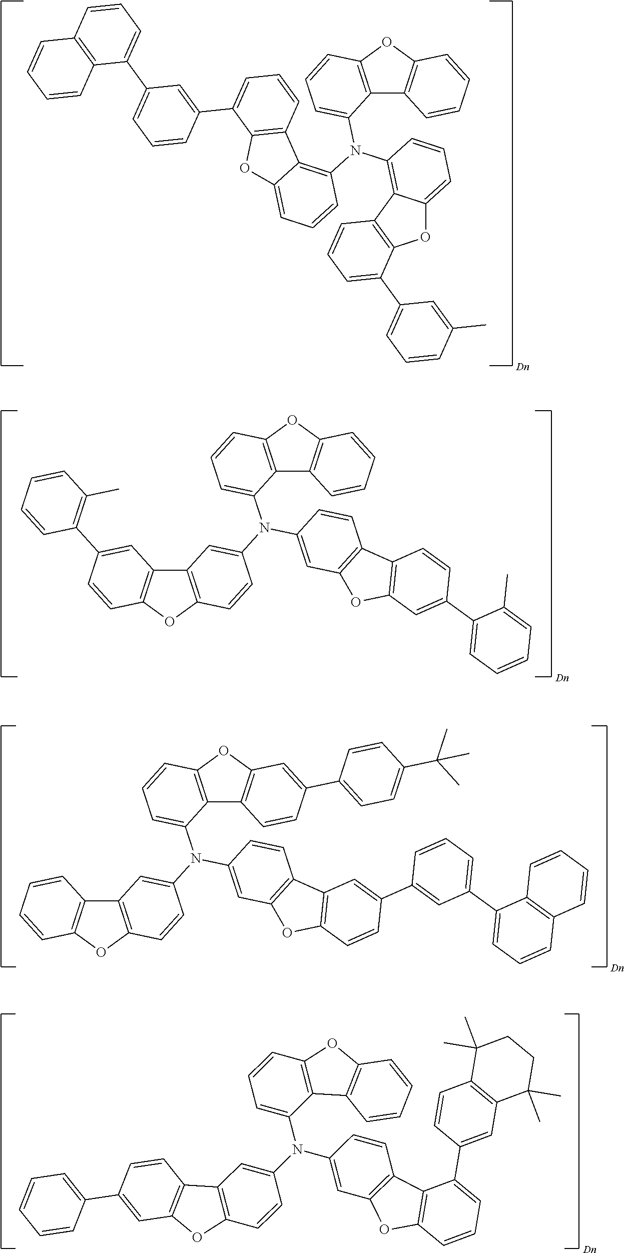 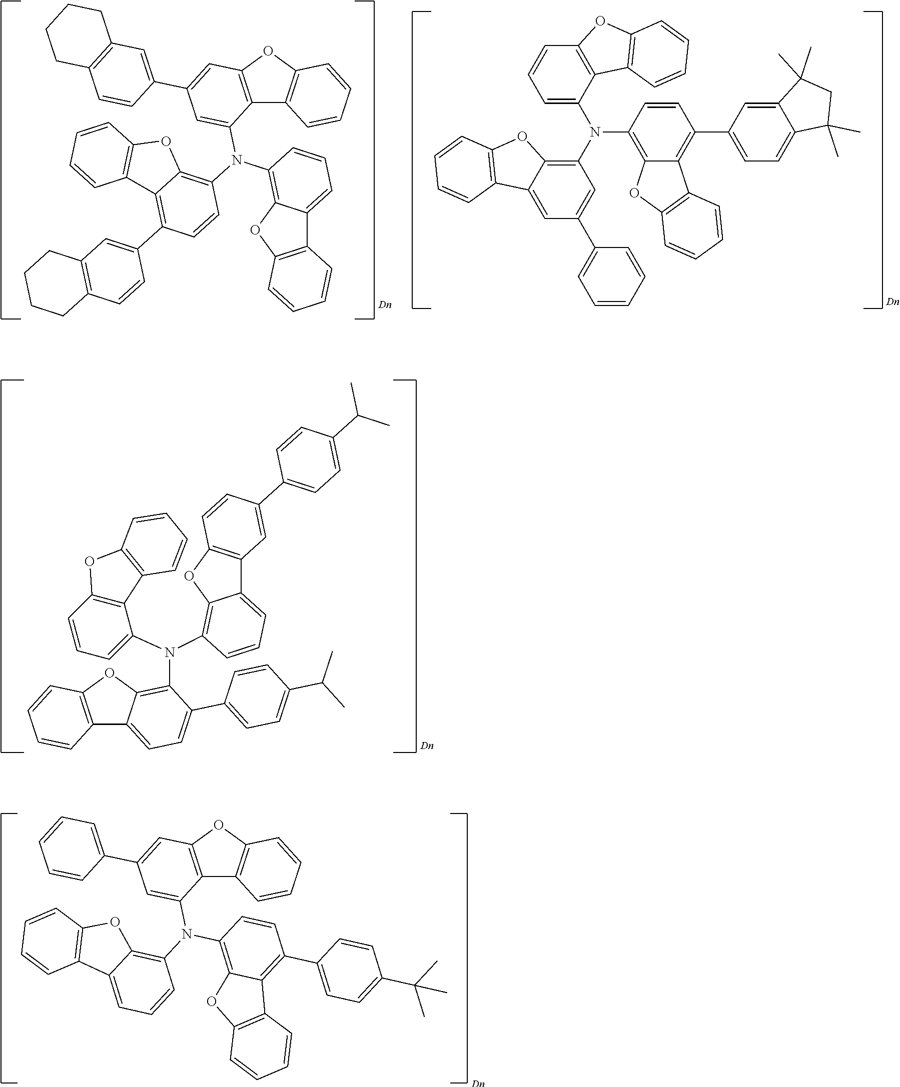 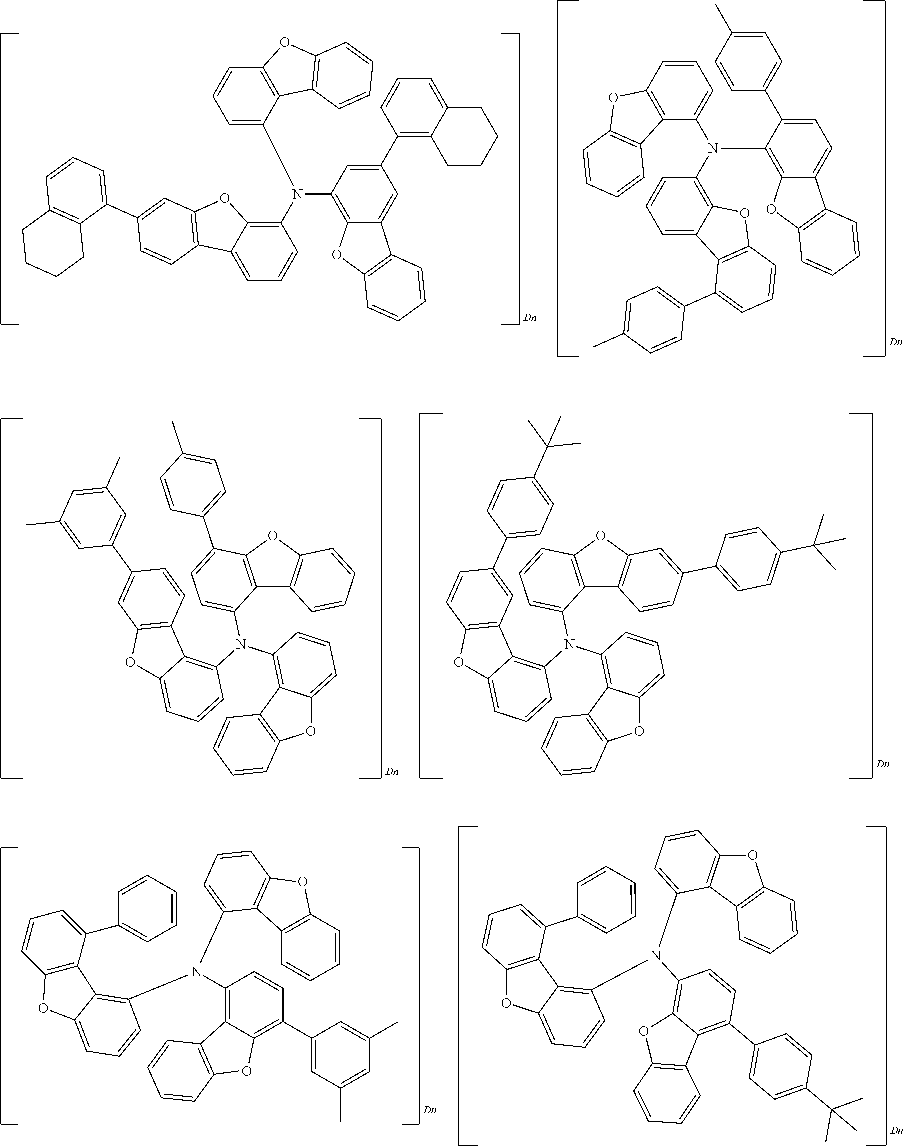 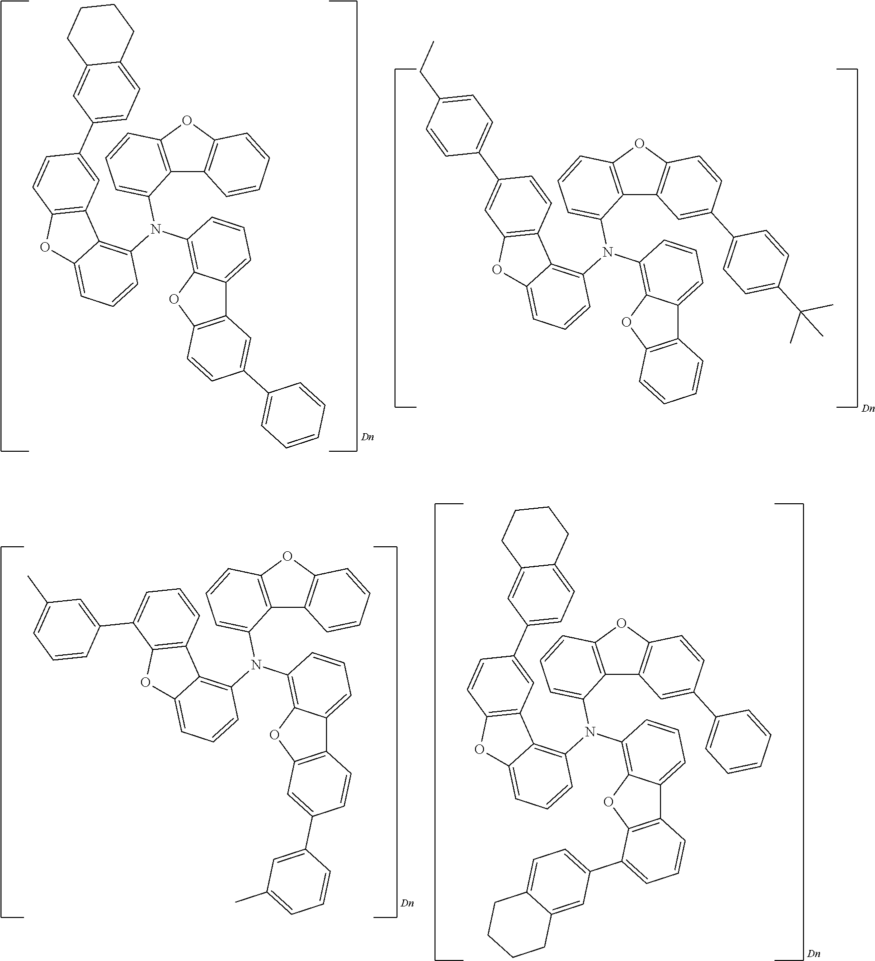 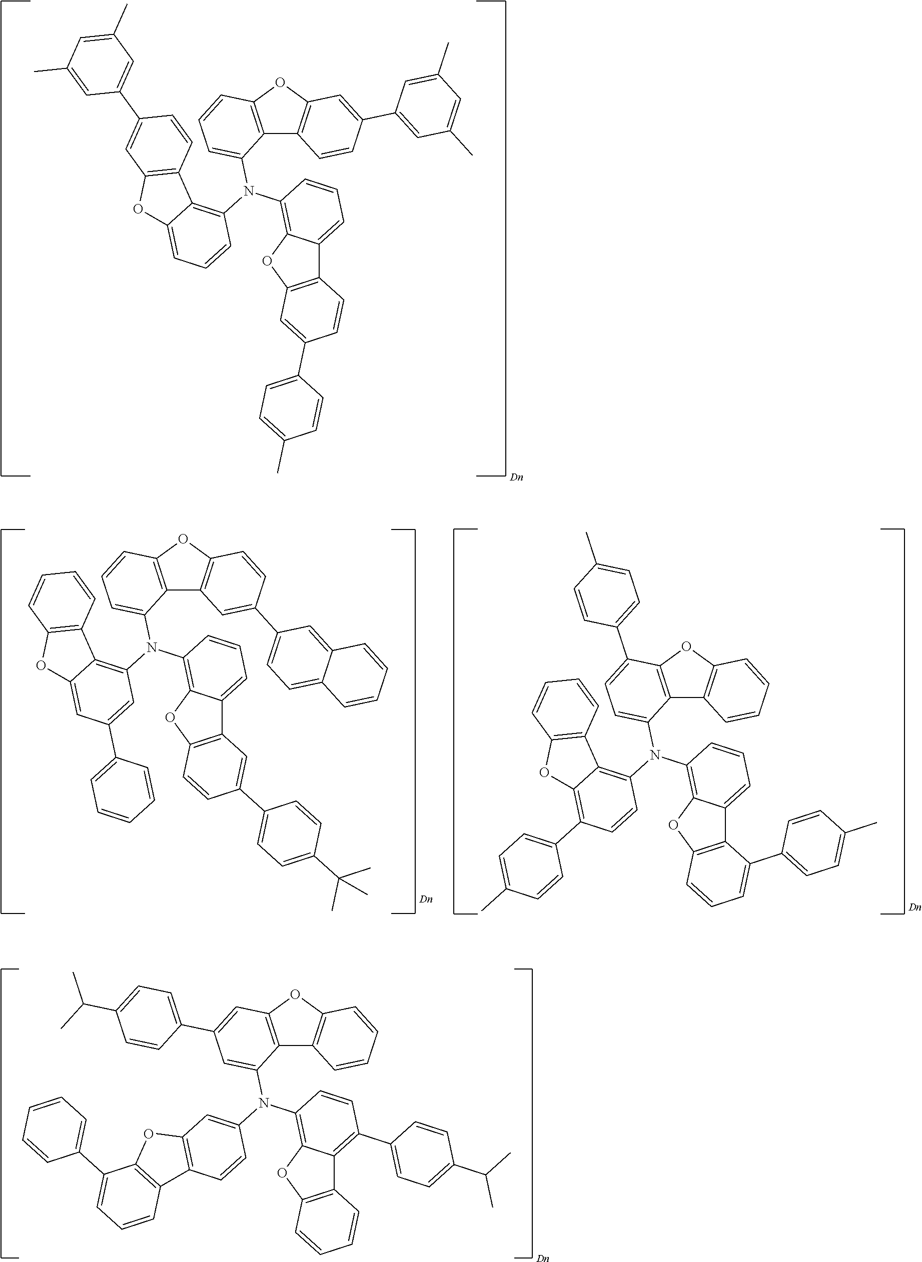 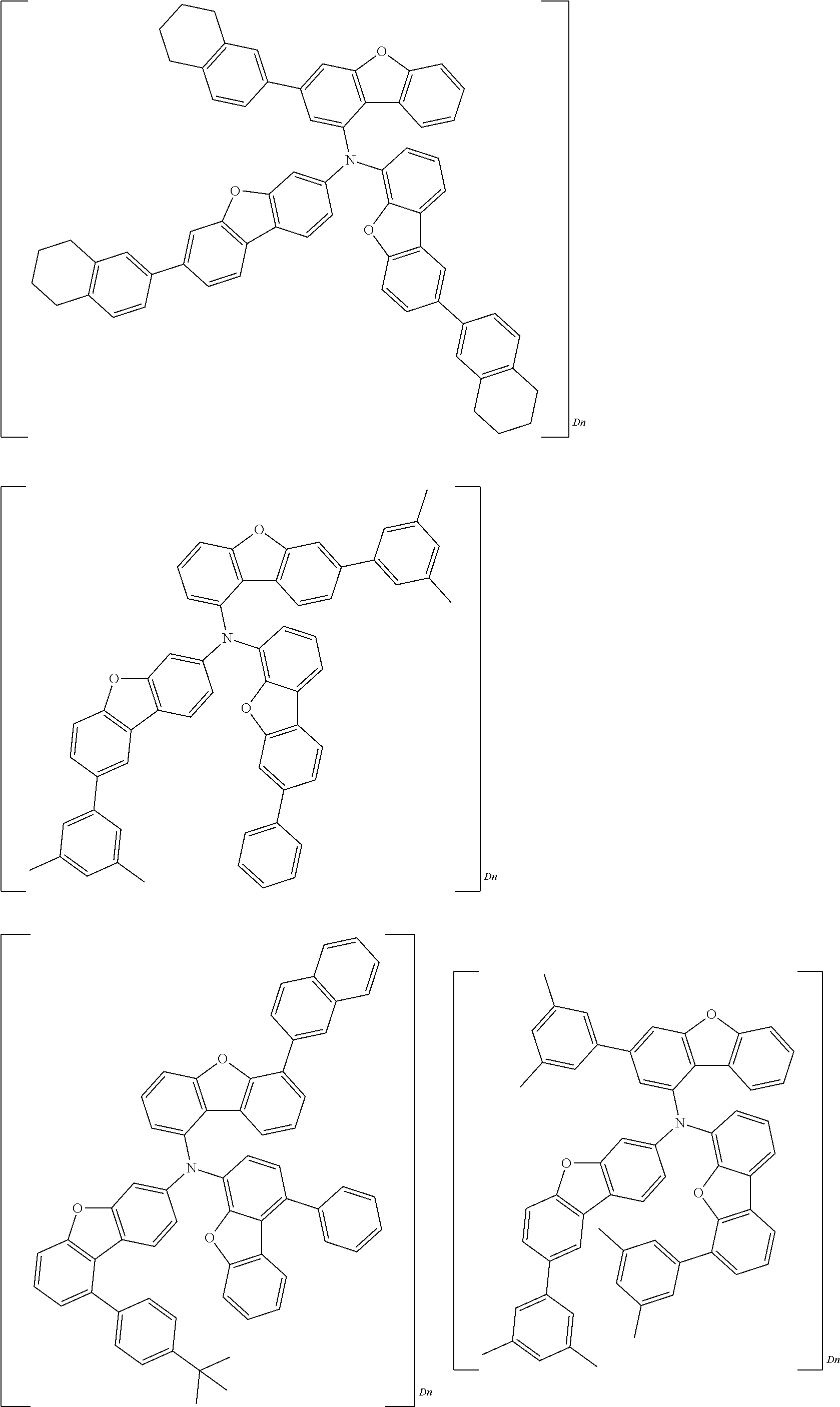 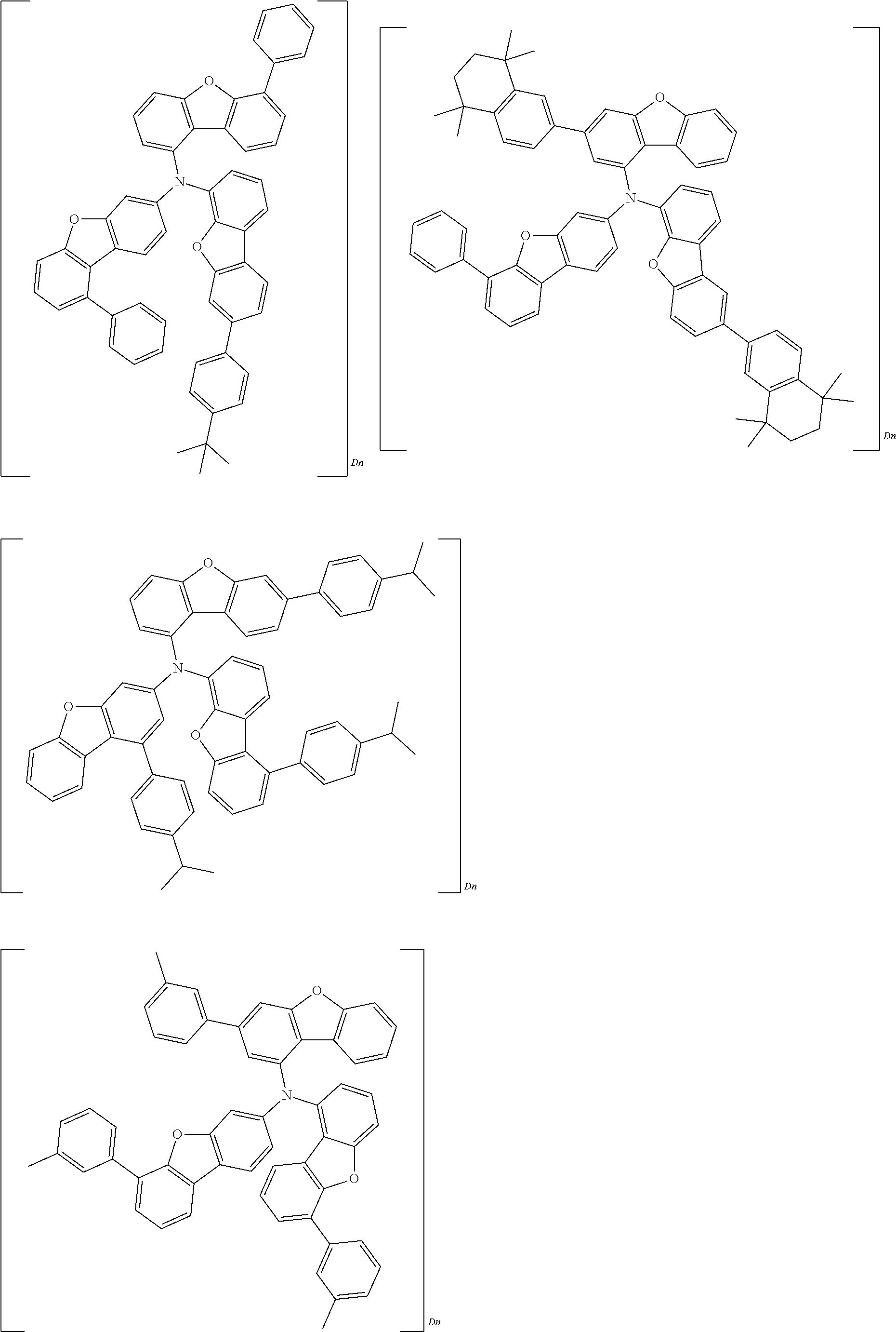 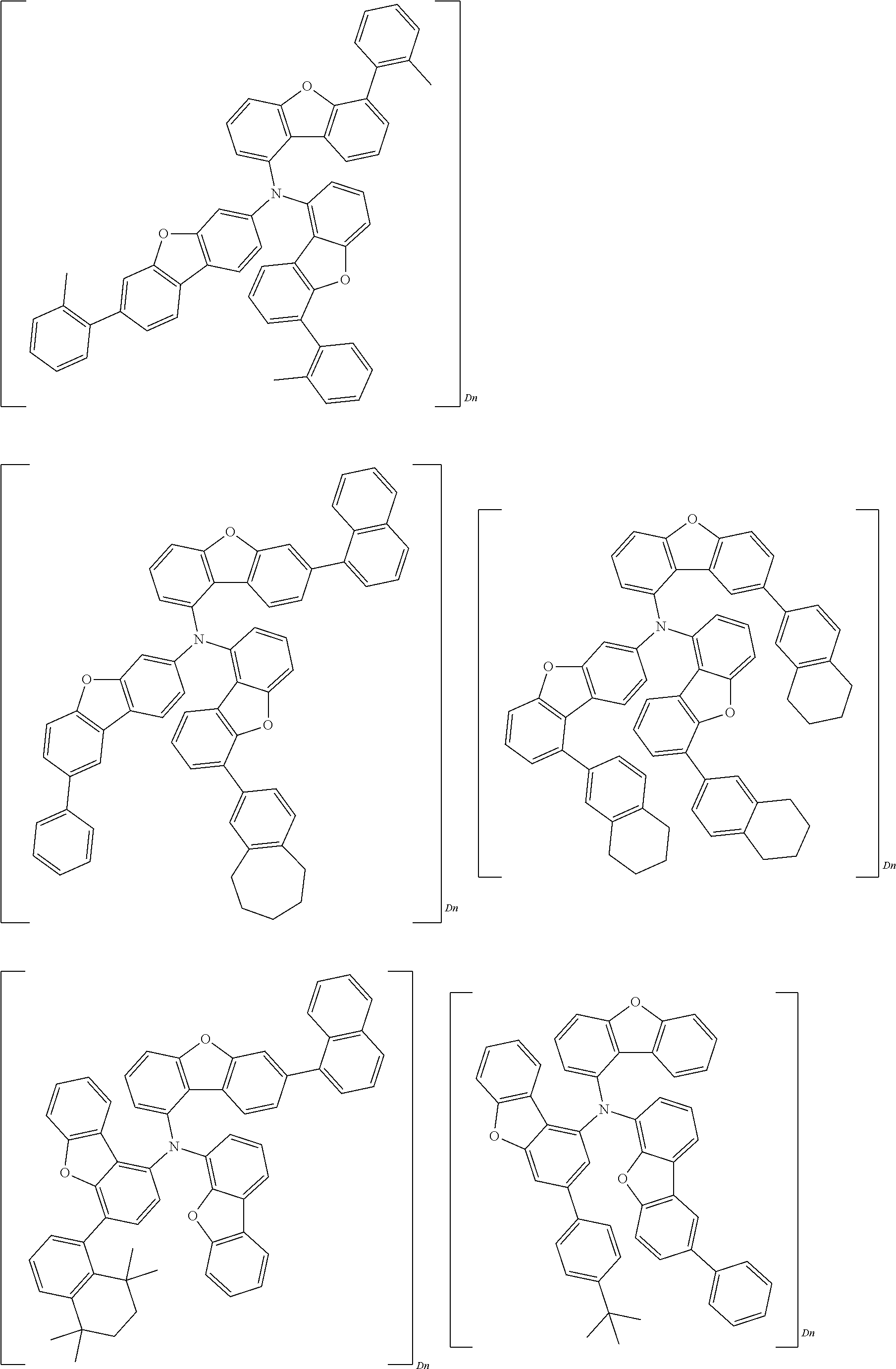 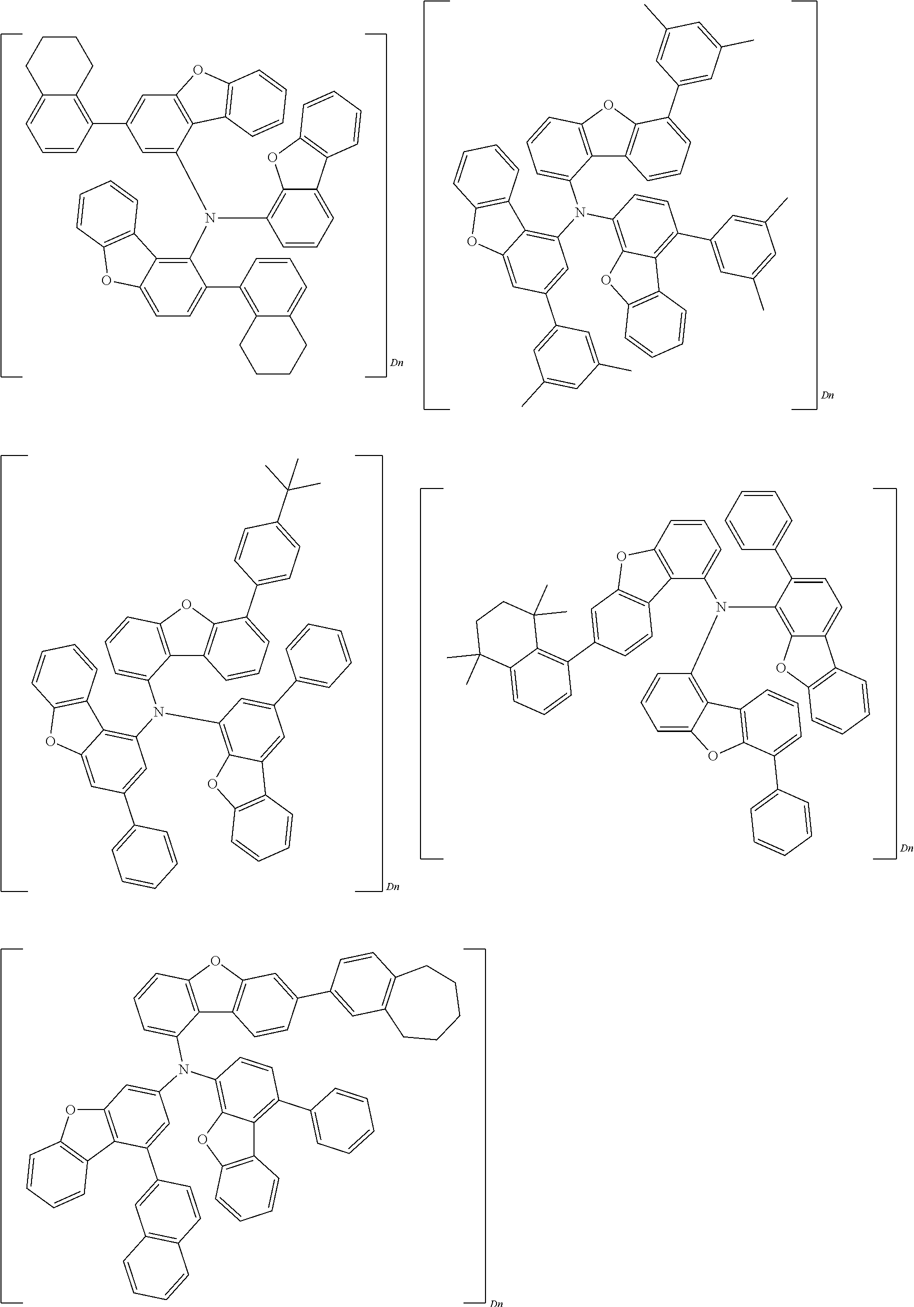 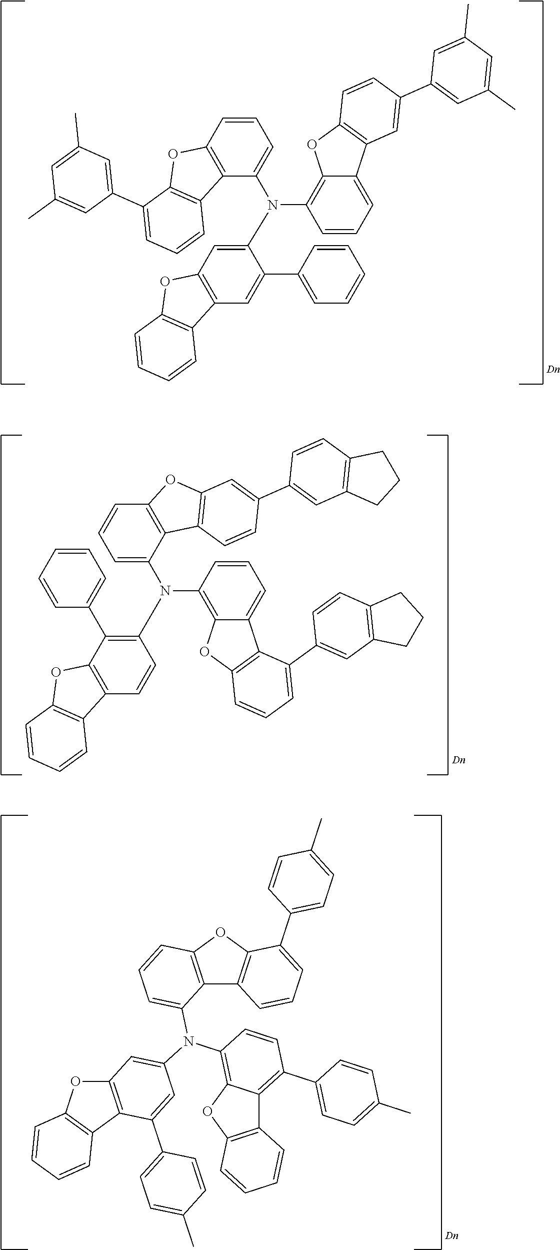 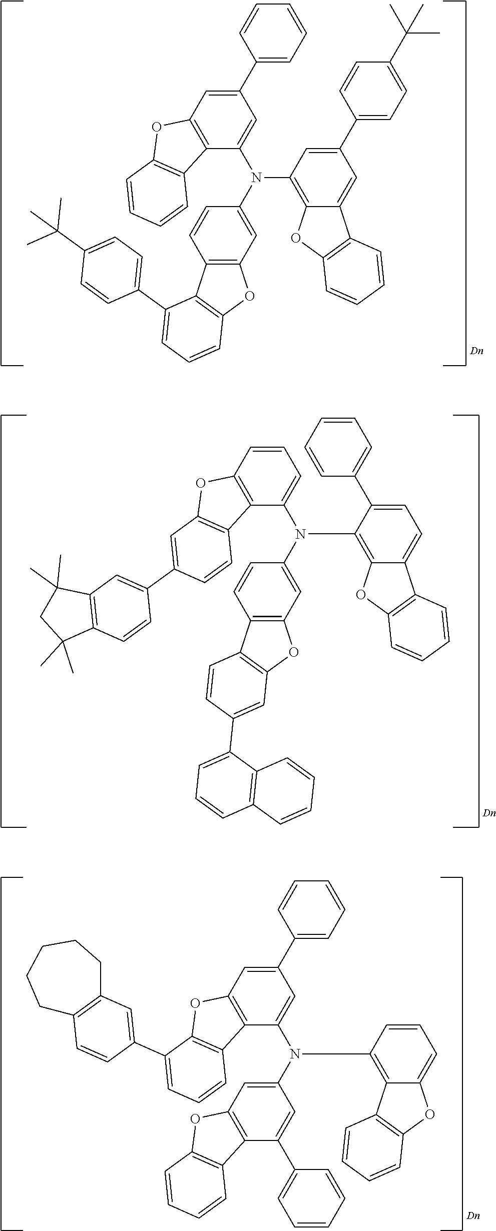 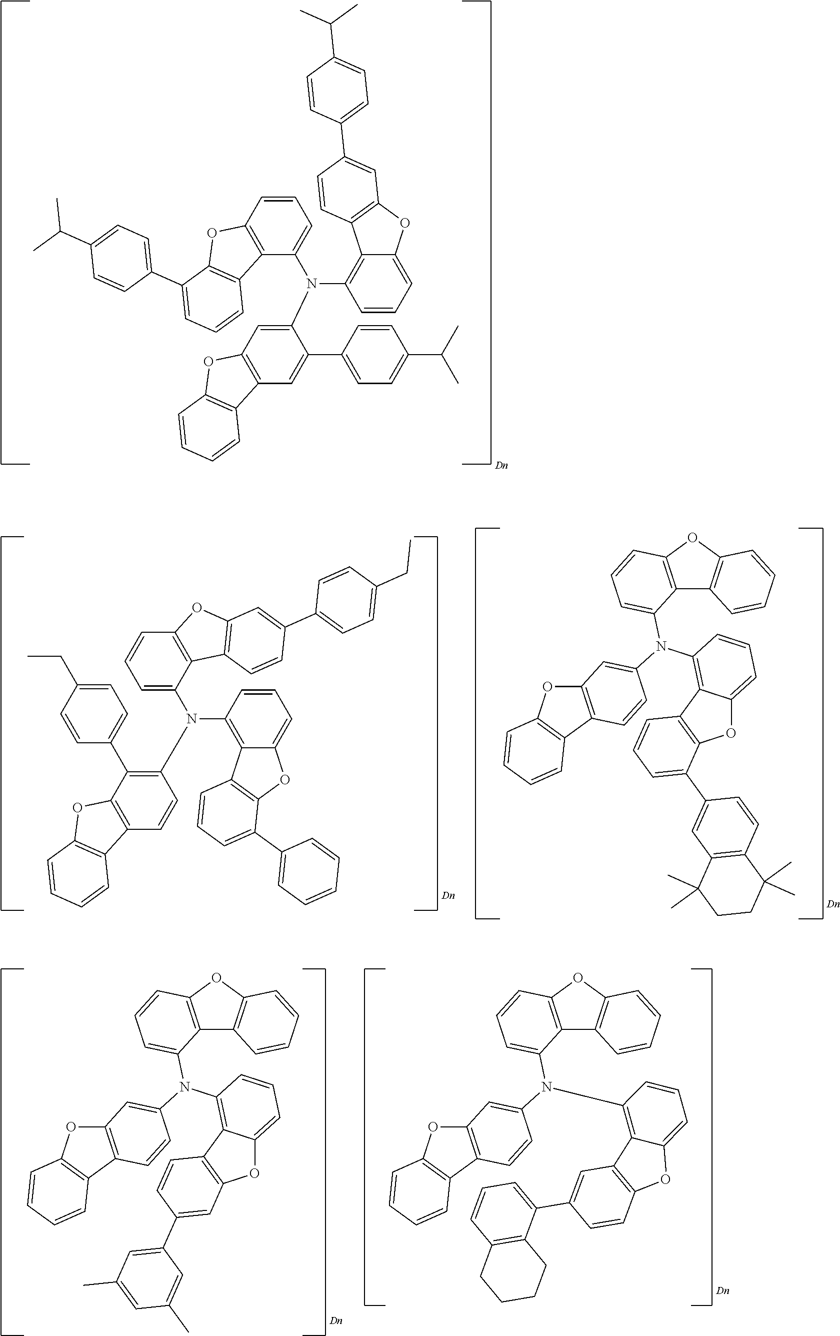 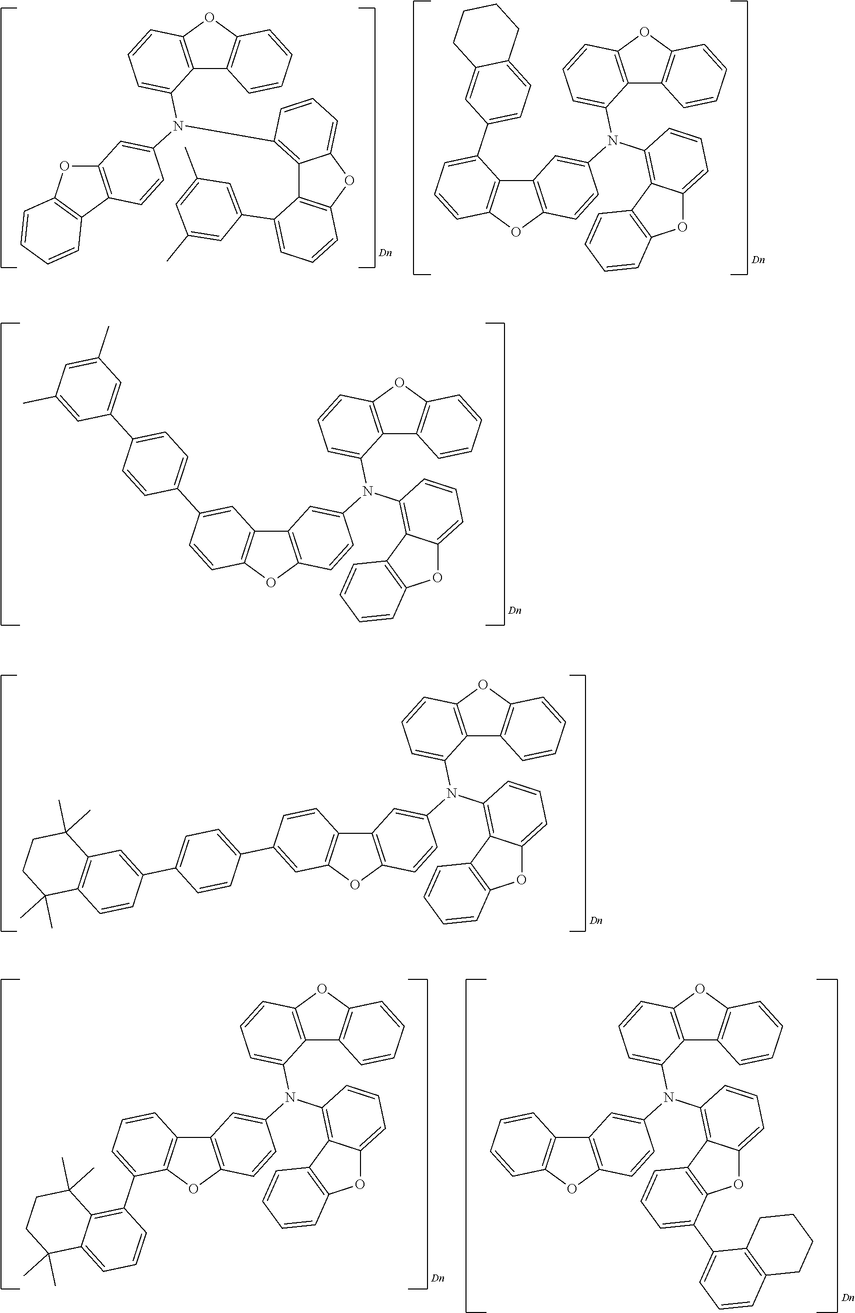 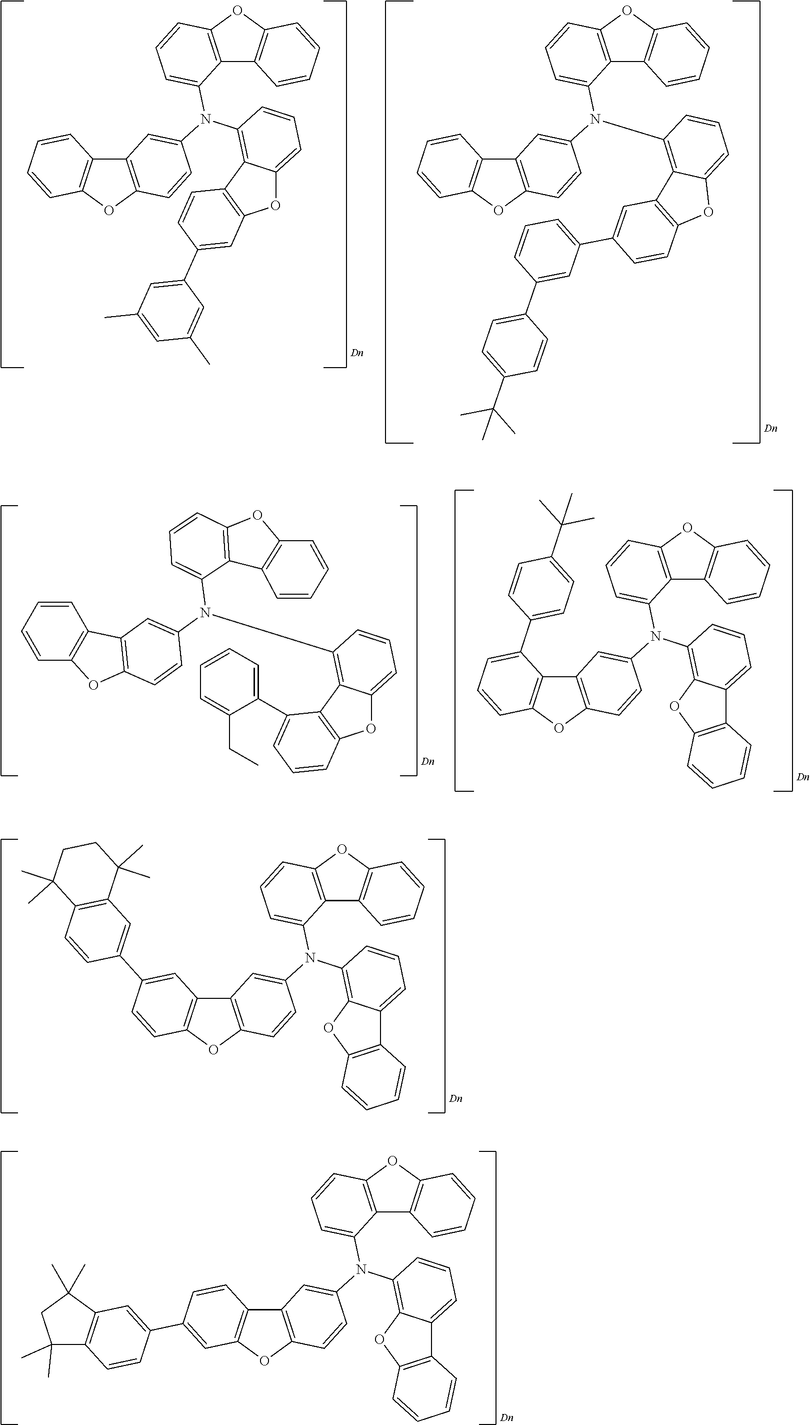 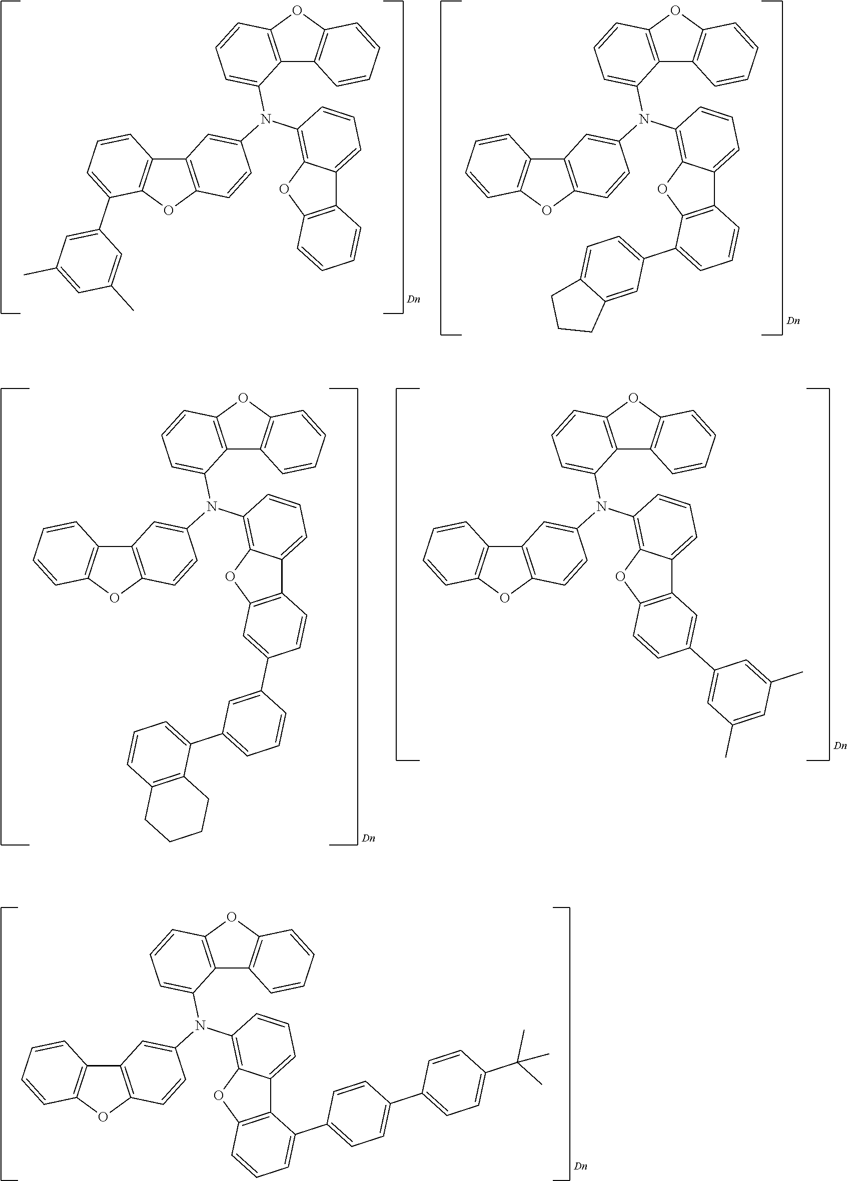 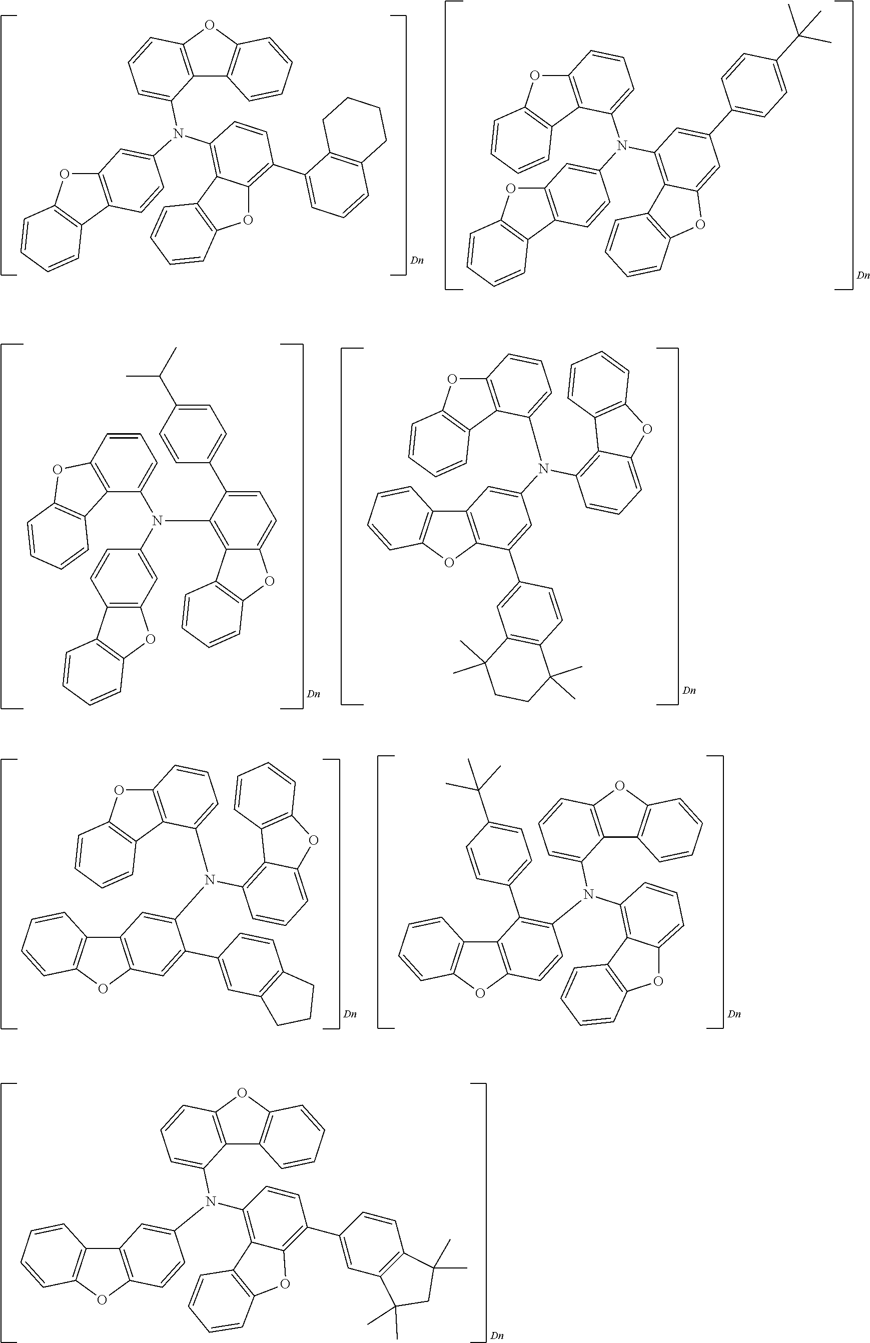 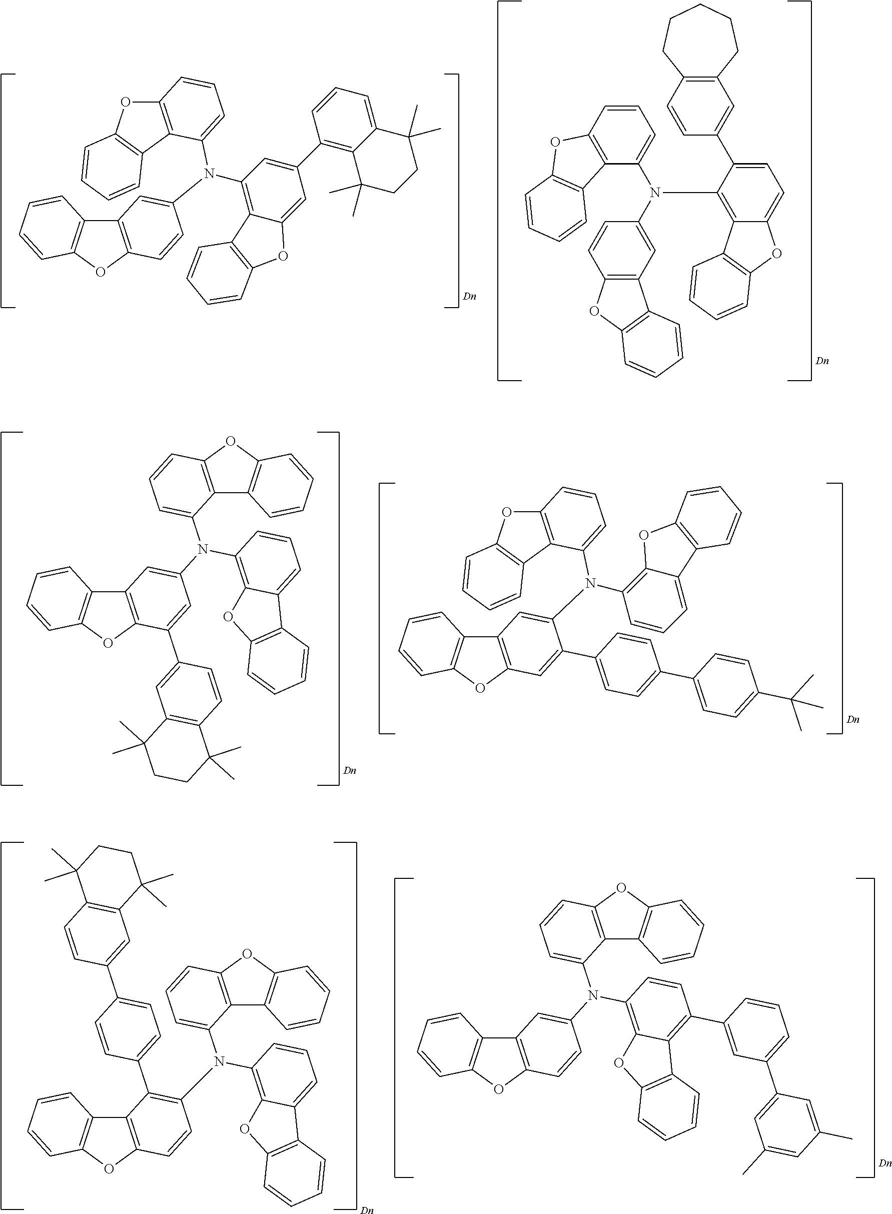 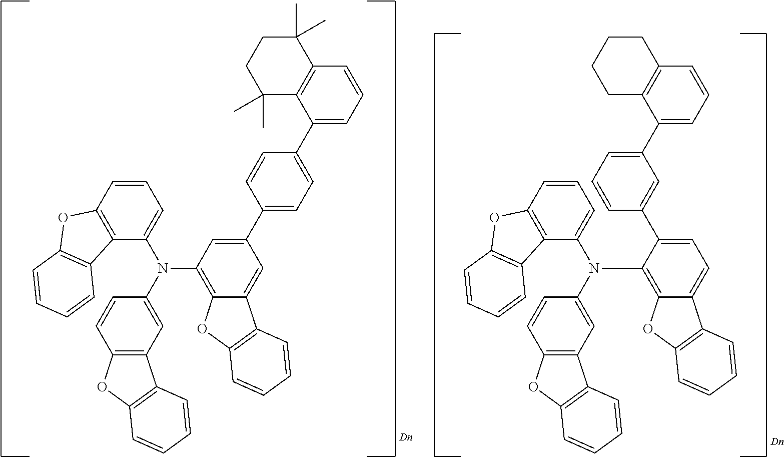
wherein the Dn means the number of deuterium (D) substitutions of each compound described in the square brackets, which is different for each compound, n is an integer of 0 or more, and the maximum value of n is the total number of hydrogens contained in each compound in the square brackets.

6. An organic light emitting device comprising: a first electrode; a second electrode that is provided opposite to the first electrode; and one or more organic material layers that are provided between the first electrode and the second electrode, wherein at least one layer of the organic material layers comprises the compound of claim 1.

7. The organic light emitting device as claimed in claim 6, wherein:
the organic material layer comprising the compound is a hole transport auxiliary layer.
